(19) United States
(12) Patent Application Publication
(10) Pub. No.: US 2026/0146252 A1
(43) Pub. Date: May 28, 2026

(54) COMPOSITIONS AND METHODS FOR ENHANCED INTESTINAL ABSORPTION OF CONJUGATED OLIGOMERIC COMPOUNDS

(71) Applicant: Ionis Pharmaceuticals, Inc., Carlsbad, CA (US)

(72) Inventors: Thazha P. Prakash, Carlsbad, CA (US); Punit P. Seth, Carlsbad, CA (US); Eric E. Swayze, Carlsbad, CA (US); Stanley T. Crooke, Carlsbad, CA (US)

(73) Assignee: Ionis Pharmaceuticals, Inc., Carlsbad, CA (US)

(21) Appl. No.: 19/458,402

(22) Filed: Jan. 23, 2026

Related U.S. Application Data
(63) Continuation of application No. 18/061,371, filed on Dec. 2, 2022, now abandoned.
Application No. 18/061,371 is a continuation of application No. 17/724,208, filed on Apr. 19, 2022, now abandoned.
Application No. 17/724,208 is a continuation of application No. 16/449,093, filed on Jun. 21, 2019, now abandoned.
Application No. 16/449,093 is a continuation of application No. 15/876,454, filed on Jan. 22, 2018, now abandoned.
Application No. 15/876,454 is a continuation of application No. 15/315,672, now abandoned.
(60) Provisional application No. 62/009,119, filed on Jun. 6, 2014.
(60) Provisional application No. 62/032,286, filed on Aug. 1, 2014.
(60) Provisional application No. 62/062,778, filed on Oct. 10, 2014.

(51) Int. Cl. C12N 15/113; A61K 9/00; A61K 31/7008; A61K 31/7105; A61K 31/7115; A61K 31/712; A61K 31/713; A61K 47/12; A61K 47/54

(52) U.S. Cl. CPC C12N 15/1138; A61K 9/0031; A61K 9/0053; A61K 31/7008; A61K 31/7105; A61K 31/7115; A61K 31/712; A61K 31/713; A61K 47/12; A61K 47/549; C12N 15/113; C12N 15/1137; C12N 2310/11; C12N 2310/315; C12N 2310/321; C12N 2310/322; C12N 2310/3231; C12N 2310/3341; C12N 2310/341; C12N 2310/346; C12N 2310/351; C12N 2310/3515; C12N 2320/32; C12N 2320/51

(57) ABSTRACT

Provided herein are compositions and methods for non-parenteral delivery of conjugated oligomeric compounds. In certain embodiments, compositions and methods are provided for oral delivery of conjugated oligomeric compounds. In certain embodiments, the oligomeric compounds are conjugated to one or more N-acetylgalactosamines or N-acetylgalactosamine analogues.

### SEQUENCE LISTING

The present application is being filed along with a Sequence Listing in electronic format. The Sequence Listing is provided as a file entitled IONIS_258C5.XML, created on Jan. 23, 2026, which is 219,555 bytes in size. The information in the electronic format of the sequence listing is incorporated herein by reference in its entirety.

### BACKGROUND OF THE INVENTION

The principle behind antisense technology is that an antisense compound hybridizes to a target nucleic acid and modulates the amount, activity, and/or function of the target nucleic acid. For example in certain instances, antisense compounds result in altered transcription or translation of a target. Such modulation of expression can be achieved by, for example, target mRNA degradation or occupancy-based inhibition. An example of modulation of RNA target function by degradation is RNase H-based degradation of the target RNA upon hybridization with a DNA-like antisense compound. Another example of modulation of gene expression by target degradation is RNA interference (RNAi). RNAi refers to antisense-mediated gene silencing through a mechanism that utilizes the RNA-induced silencing complex (RISC). An additional example of modulation of RNA target function is by an occupancy-based mechanism such as is employed naturally by microRNA. MicroRNAs are small non-coding RNAs that regulate the expression of protein-coding RNAs. The binding of an antisense compound to a microRNA prevents that microRNA from binding to its messenger RNA targets, and thus interferes with the function of the microRNA. MicroRNA mimics can enhance native microRNA function. Certain antisense compounds alter splicing of pre-mRNA. Regardless of the specific mechanism, sequence-specificity makes antisense compounds attractive as tools for target validation and gene functionalization, as well as therapeutics to selectively modulate the expression of genes involved in the pathogenesis of diseases.

Antisense technology is an effective means for modulating the expression of one or more specific gene products and can therefore prove to be uniquely useful in a number of therapeutic, diagnostic, and research applications. Chemically modified nucleosides may be incorporated into antisense compounds to enhance one or more properties, such as nuclease resistance, pharmacokinetics or affinity for a target nucleic acid. In 1998, the antisense compound, Vitravene® (fomivirsen; developed by Isis Pharmaceuticals Inc., Carlsbad, CA) was the first antisense drug to achieve marketing clearance from the U.S. Food and Drug Administration (FDA), and is currently a treatment of cytomegalovirus (CMV)-induced retinitis in AIDS patients. For another example, an antisense oligonucleotide targeting ApoB, KYNAMRO™, has been approved by the U.S. Food and Drug Administration (FDA) as an adjunct treatment to lipid-lowering medications and diet to reduce low density lipoprotein-cholesterol (LDL-C), ApoB, total cholesterol (TC), and non-high density lipoprotein-cholesterol (non HDL-C) in patients with homozygous familial hypercholesterolemia (HoFH).

New chemical modifications have improved the potency and efficacy of antisense compounds, uncovering the potential for oral delivery as well as enhancing subcutaneous administration, decreasing potential for side effects, and leading to improvements in patient convenience. Chemical modifications increasing potency of antisense compounds allow administration of lower doses, which reduces the potential for toxicity, as well as decreasing overall cost of therapy. Modifications increasing the resistance to degradation result in slower clearance from the body, allowing for less frequent dosing. Different types of chemical modifications can be combined in one compound to further optimize the compound's efficacy.

Advances in the field of biotechnology have led to significant advances in the treatment of diseases such as cancer, genetic diseases, arthritis and AIDS that were previously difficult to treat. Many such advances involve the administration of oligonucleotides and other nucleic acids to a subject, particularly a human subject. The administration of such molecules via parenteral routes has been shown to be effective for the treatment of diseases and/or disorders. See, e.g., Draper et al., U.S. Pat. No. 5,595,978, Jan. 21, 1997, which discloses intravitreal injection as a means for the direct delivery of antisense oligonucleotides to the vitreous humor of the mammalian eye. See also, Robertson, Nature Biotechnology, 1997, 15, 209, and Genetic Engineering News, 1997, 15, 1, each of which discuss the treatment of Crohn's disease via intravenous infusions of antisense oligonucleotides. Non-parenteral routes for administration of oligonucleotides and other nucleic acids (such as oral or rectal delivery or other mucosal routes) offers the promise of simpler, easier and less injurious administration of such nucleic acids without the need for sterile procedures and their concomitant expenses, e.g., hospitalization and/or physician fees. However, the absorption of non-parenterally administered drugs is often poor. There thus is a need to provide compositions and methods to enhance the availability of novel drugs such as oligonucleotides when administered via non-parenteral routes. It is desirable that such new compositions and methods provide for the simple, convenient, practical and optimal non-parenteral delivery of oligonucleotides and other nucleic acids.

Oral administration of drugs, including oligomeric compounds such as antisense oligonucleotides and other nucleic acids, offers the promise of simpler, easier and less injurious administration without the need for sterile procedures and their concomitant expenses, e.g., hospitalization and/or physician fees. However, the absorption of orally administered drugs is often poor. One approach to enhancing the absorption of orally administered drugs is pulsatile release formulations in which multiple doses of drug are released from a single formulation by the use of delayed release coatings (U.S. Pat. Nos. 7,576,067, 5,508,040, 6,117,450, 5,840,329, 5,814,336, and 5,686,105, the entire contents of which are incorporated herein by reference). There is a need to provide compositions and methods to enhance the absorption and/or bioavailability of orally administered drugs, particularly oligonucleotides. Pharmaceutical compositions comprising an antisense oligonucleotides targeted SMAD7 have also been disclosed for oral administration (U.S. Pat. No. 8,648,186, the entire contents of which are incorporated herein by reference).

In one animal assay rats fed a standard pelleted diet enriched with sodium decanoate and gapped LNA oligonucleotide targeted to human and mouse apoB mRNA demonstrated a statistically significant dose dependent reduction in total serum cholesterol after one week. The reduced total serum cholesterol was maintained over the course of the dosing period (Hardee et al., Arteriosclerosis, Thrombosis, and Vascular Biology, 2010 Scientific Sessions, April 8-10, San Francisco, California, poster P358).

Various routes and formulations for the delivery of antisense oligonucleotides has also been previously described (“Antisense Drug Technology, Principles, Strategies, and Applications” Edited by Stanley T. Crooke, 2008, Chapter 8, CRC Press, Boca Raton, Florida; and Sambrook et al.).

One approach to oral delivery of poorly absorbed drugs such as polar molecules and bioactive peptides and proteins utilizes a mucoadhesive patch system for drug delivery (PCT International application WO 03/007913 A2, published on Jan. 30, 2003, the entire contents of which are incorporated herein by reference).

Oral delivery of low-molecular weight heparin has been achieved by conjugation of the haparin to deoxycholic acid (Lee, et al., Circulation, Journal of the American Heart Association, 2001, 104, 3116-3120).

The use of mucus-penetrating nanoparticles for drug and gene delivery to mucosal tissues is another non-parenteral delivery system that is being examined (Lai et al., Adv. Drug Deliv. Rev., 2009, 61 (2). 158-171).

Antisense drugs have also been administered by enema in a successful human clinical trial targeting pouchitis (U.S. Pat. No. 8,084,432, the entire contents of which are incorporated herein by reference). There is a need to provide compositions and methods to enhance the absorption and/or bioavailability of rectally administered drugs, particularly oligonucleotides.

### SUMMARY OF THE INVENTION

In certain embodiments, the present disclosure provides methods and compositions for non-parenteral delivery of oligomeric compounds. In certain embodiments, the present disclosure provides methods and compositions for non-parenteral delivery of oligomeric compounds that result in a reduction in the amount or activity of a nucleic acid transcript in a cell. In certain embodiments, the present disclosure provides methods and compositions for oral delivery of oligomeric compounds. In certain embodiments, the present disclosure provides methods and compositions for oral delivery of oligomeric compounds that result in a reduction in the amount or activity of a nucleic acid transcript in a cell. In certain embodiments, the present disclosure provides compositions comprising conjugated oligomeric compounds. In certain embodiments, the present disclosure provides compositions comprising conjugated antisense compounds. In certain embodiments, the present disclosure provides compositions comprising conjugated antisense compounds comprising an antisense oligonucleotide complementary to a nucleic acid transcript.

In certain embodiments, the conjugate group of the conjugated oligomeric compounds are targeted to the asialoglycoprotein receptor. The asialoglycoprotein receptor (ASGP-R) has been described previously. See e.g., Park et al., PNAS vol. 102, No. 47, pp 17125-17129 (2005). Such receptors are expressed on liver cells, particularly hepatocytes. Further, it has been shown that compounds comprising clusters of three N-acetylgalactosamine (GalNAc) ligands are capable of binding to the ASGP-R, resulting in uptake of the compound into the cell. See e.g., Khorev et al., Bioorganic and Medicinal Chemistry, 16, 9, pp 5216-5231 (May 2008). Accordingly, conjugates comprising such GalNAc clusters have been used to facilitate uptake of certain compounds into liver cells, specifically hepatocytes. For example it has been shown that certain GalNAc-containing conjugates increase activity of duplex siRNA compounds in liver cells in vivo. In such instances, the GalNAc-containing conjugate is typically attached to the sense strand of the siRNA duplex. Since the sense strand is discarded before the antisense strand ultimately hybridizes with the target nucleic acid, there is little concern that the conjugate will interfere with activity. Typically, the conjugate is attached to the 3′ end of the sense strand of the siRNA. See e.g., U.S. Pat. No. 8,106,022. Certain conjugate groups described herein are more active and/or easier to synthesize than conjugate groups previously described.

In certain embodiments of the present invention, conjugates are attached to single-stranded antisense compounds such as antisense oligonucleotides, including, but not limited to RNase H based antisense compounds and antisense compounds that alter splicing of a pre-mRNA target nucleic acid. In such embodiments, the conjugate should remain attached to the antisense compound long enough to provide benefit (improved uptake into cells) but then should either be cleaved, or otherwise not interfere with the subsequent steps necessary for activity, such as hybridization to a target nucleic acid and interaction with RNase H or enzymes associated with splicing or splice modulation. This balance of properties is more important in the setting of single-stranded antisense compounds than in siRNA compounds, where the conjugate may simply be attached to the sense strand. Disclosed herein are conjugated single-stranded antisense compounds having improved potency in liver cells in vivo compared with the same antisense compound lacking the conjugate. Given the required balance of properties for these compounds such improved potency is surprising.

In certain embodiments, conjugate groups herein comprise a cleavable moiety. As noted, without wishing to be bound by mechanism, it is logical that the conjugate should remain on the compound long enough to provide enhancement in uptake, but after that, it is desirable for some portion or, ideally, all of the conjugate to be cleaved, releasing the parent compound (e.g., antisense compound) in its most active form. In certain embodiments, the cleavable moiety is a cleavable nucleoside. Such embodiments take advantage of endogenous nucleases in the cell by attaching the rest of the conjugate (the cluster) to the antisense oligonucleotide through a nucleoside via one or more cleavable bonds, such as those of a phosphodiester linkage. In certain embodiments, the cluster is bound to the cleavable nucleoside through a phosphodiester linkage. In certain embodiments, the cleavable nucleoside is attached to the antisense oligonucleotide (antisense compound) by a phosphodiester linkage. In certain embodiments, the conjugate group may comprise two or three cleavable nucleosides. In such embodiments, such cleavable nucleosides are linked to one another, to the antisense compound and/or to the cluster via cleavable bonds (such as those of a phosphodiester linkage). Certain conjugates herein do not comprise a cleavable nucleoside and instead comprise a cleavable bond. It is shown that that sufficient cleavage of the conjugate from the oligonucleotide is provided by at least one bond that is vulnerable to cleavage in the cell (a cleavable bond).

In certain embodiments, conjugated antisense compounds are prodrugs. Such prodrugs are administered to an animal and are ultimately metabolized to a more active form. For example, conjugated antisense compounds are cleaved to remove all or part of the conjugate resulting in the active (or more active) form of the antisense compound lacking all or some of the conjugate.

In certain embodiments, the conjugates herein do not substantially alter certain measures of tolerability. For example, it is shown herein that conjugated antisense compounds are not more immunogenic than unconjugated parent compounds. Since potency is improved, embodiments in which tolerability remains the same (or indeed even if tolerability worsens only slightly compared to the gains in potency) have improved properties for therapy.

In certain embodiments, the conjugates herein comprise one or more modifications to the galactosyl analgues with substitutions at the anomeric, C2-, C5-, and/or C6-positions. In certain embodiments, the oxygen or hydroxyl moiety at one or more of the anomeric, C2-, C5-, and/or C6-positions is replaced with a sulfur. In certain embodiments, modification to the galactosyl analgues with substitutions at the anomeric, C2-, C5-, and/or C6-positions provides an increase in potency, efficacy, an/or stability.

In certain embodiments, conjugation of antisense compounds herein results in increased delivery, uptake and activity in hepatocytes. Thus, more compound is delivered to liver tissue.

In certain embodiments, a conjugated oligomeric compound has a structure selected from among the following:

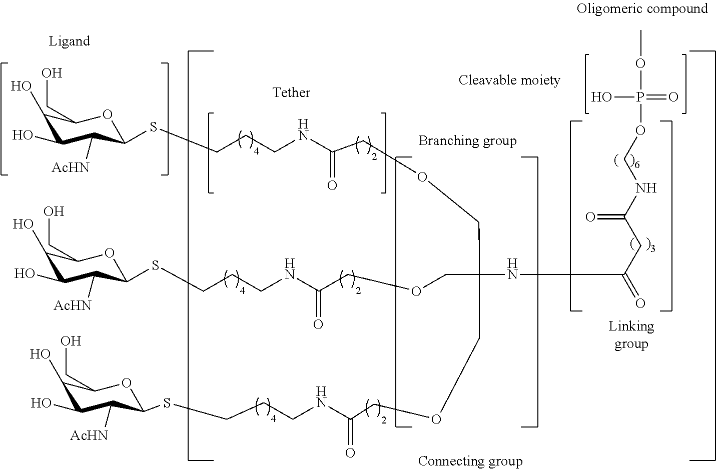

The present disclosure provides the following non-limiting numbered embodiments:

Embodiment 1. A composition for non-parental administration comprising:
a conjugated oligomeric compound; and
an excipient.

Embodiment 2. The composition of embodiment 1, wherein the conjugate group comprises from 1 to 3 moieties having Formula I:

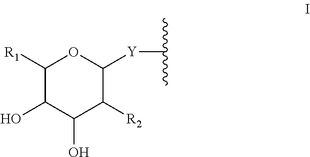

wherein independently for each moiety having formula I:
R₁ is selected from Q₁, CH₂Q₁, CH₂OH, CH₂NJ₁J₂, CH₂N₃ and CH₂SJ₃;
Q₁ is selected from aryl, substituted aryl, heterocyclic, substituted heterocyclic, heteroaryl and substituted heteroaryl;
R₂ is selected from N₃, CN, halogen, N(H)C(═O)-Q₂, substituted thiol, aryl, substituted aryl, heterocyclic, substituted heterocyclic, heteroaryl and substituted heteroaryl;
Q₂ is selected from H, C₁-C₆ alkyl, substituted C₁-C₆ alkyl, C₁-C₆ alkoxy, substituted C₁-C₆ alkoxy, aryl, substituted aryl, heterocyclic, substituted heterocyclic, heteroaryl and substituted heteroaryl;
Y is selected from O, S, CJ₄J₅, NJ₆ and N(J₆)C(═O);
J₁, J₂, J₃, J₄, J₅, and J₆ are each, independently, H or a substituent group; and
each substituent group is, independently, mono or poly substituted with optionally protected substituent groups independently selected from halogen, C₁-C₆ alkyl, C₁-C₆ alkoxy, aryl, heterocyclic and heteroaryl wherein each substituent group can include a linear or branched alkylene group optionally including one or more groups independently selected from O, S, NH and C(═O), and wherein each substituent group may be further substituted with one or more groups independently selected from C₁-C₆ alkyl, halogen or C₁-C₆ alkoxy wherein each cyclic group is mono or polycyclic.

Embodiment 3. The composition of embodiment 2, comprising one moiety having Formula I that is linked to the oligomeric compound through a connecting group.

Embodiment 4. The composition of embodiment 2 comprising 2 or 3 moieties of formula 1 that are linked to the oligomeric compound through a connecting group that comprises a branching group.

Embodiment 5. The composition of any of embodiments 2 to 4, wherein each R₁ is, independently, selected from Q₁ and CH₂Q₁.

Embodiment 6. The composition of any of embodiments 2 to 4, wherein each Q₁ is substituted heteroaryl.

Embodiment 7. The composition of embodiment 6, wherein each Q₁ is selected from among:

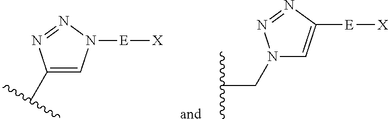

wherein:
E is a single bond or one of said linear or branched alkylene groups; and
X is H or one of said substituent groups.

Embodiment 8. The composition of embodiment 7, wherein each X is selected from substituted aryl and substituted heteroaryl.

Embodiment 9. The composition of embodiment 8, wherein each X is phenyl or substituted phenyl comprising one or more substituent groups selected from F, Cl, Br, CO₂Et, OCH₃, CN, CH₃, OCH₃, CF₃, N(CH₃)₂ and O-phenyl.

Embodiment 10. The composition of embodiment 7, wherein each Q₁ has the formula:

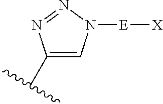

wherein:
-E-X is selected from among:

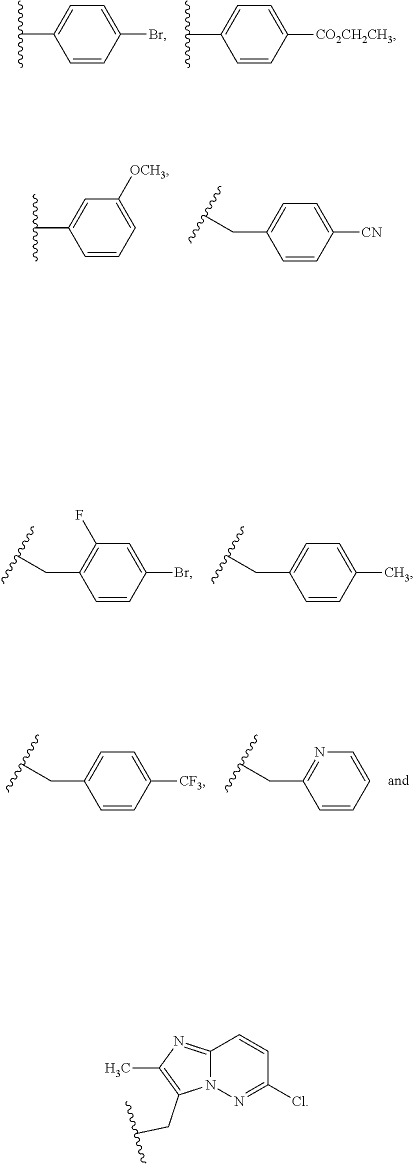

Embodiment 11. The composition of embodiment 7, wherein each Q₁ has the formula:

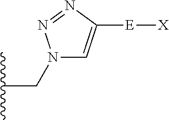

wherein:
-E-X is selected from among:

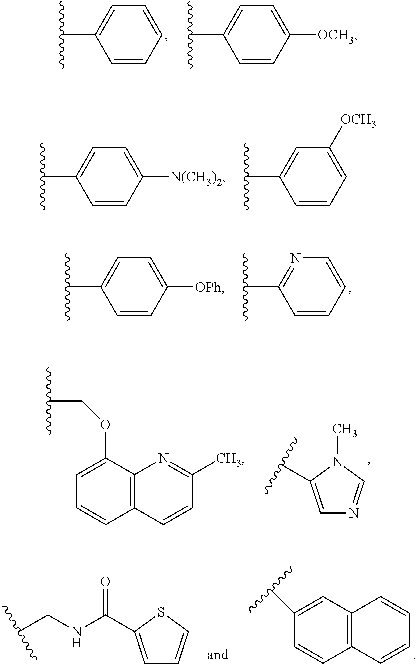

Embodiment 12. The composition of any of embodiments 2 to 4, wherein each R₁ is selected from CH₂OH, CH₂NJ₁J₂, CH₂N₃ and CH₂SJ₃ wherein J₁, J₂ and J₃ are each independently selected from H and CH₃.

Embodiment 13. The composition of embodiment 12, wherein each R₁ is CH₂OH.

Embodiment 14. The composition of embodiment 12, wherein each J₁, J₂ and J₃ are each H.

Embodiment 15. The composition of embodiment 12, wherein each J₁, J₂ and J₃ are each CH₃.

Embodiment 16. The composition of any of embodiments 2 to 15, wherein each R₂ is N(H)C(═O)-Q₂.

Embodiment 17. The composition of embodiment 16, wherein each Q₂ is selected from C₁-C₆ alkyl, substituted C₁-C₆ alkyl, C₁-C₆ alkoxy and substituted C₁-C₆ alkoxy.

Embodiment 18. The composition of embodiment 17, wherein each Q₂ is selected from CH₃, CH₂CH₃, CH(CH₃)₂, (CH₂)₂CH₃, C(CH₃)₃, CCl₃, CF₃, O—C(CH₃)₃, CH₂CO₂H, CH₂NH₂ and CH₂CF₃.

Embodiment 19. The composition of embodiment 18, wherein each Q₂ is CH₃.

Embodiment 20. The composition of embodiments 16, wherein each Q₂ is selected from aryl, substituted aryl, heterocyclic, substituted heterocyclic, heteroaryl and substituted heteroaryl.

Embodiment 21. The composition of embodiment 20, wherein each Q₂ is selected from aryl, substituted aryl, heteroaryl and substituted heteroaryl.

Embodiment 22. The composition of embodiment 21, wherein each Q₂ is selected is selected from among:

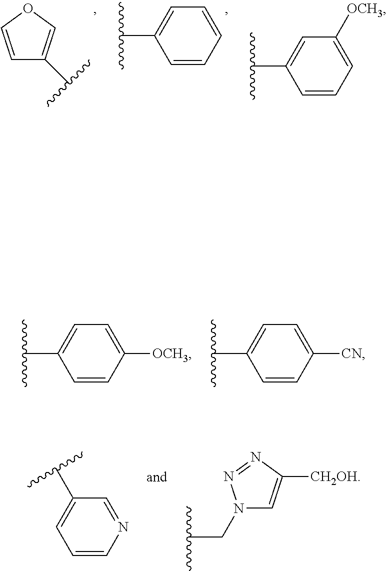

Embodiment 23. The composition of any of embodiments 2 to 15, wherein each R₂ is selected from aryl, substituted aryl, heterocyclic, substituted heterocyclic, heteroaryl and substituted heteroaryl.

Embodiment 24. The composition of embodiment 23, wherein each R₂ has the formula:

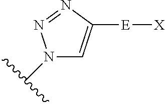

wherein:
E is a single bond or one of said linear or branched alkylene groups; and
X is H or one of said substituent groups.

Embodiment 25. The composition of embodiment 24, wherein each -E-X is selected from CH₂OH, CH₂NH₂, CH₂N(H)CH₃, CH₂N(CH₃)₂, CO₂H and CH₂NHCOCH₃.

Embodiment 26. The composition of embodiment 24, wherein each -E-X is selected from among:

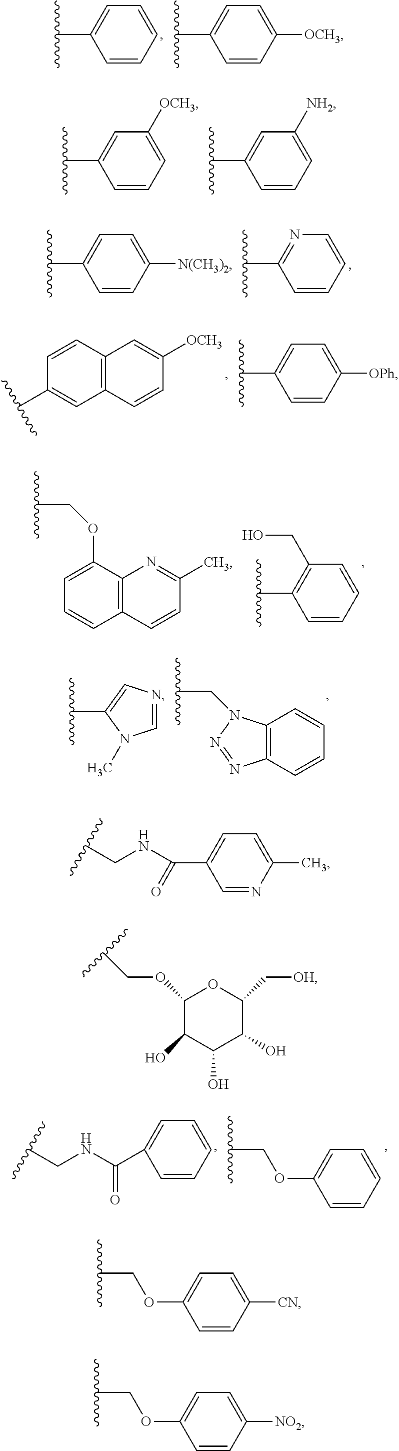

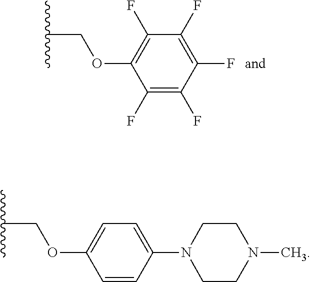 [SMILES: *COc1c(F)c(F)c(F)c(F)c1F ; *COc1ccc(N2CCN(C)CC2)cc1]

Embodiment 27. The composition of any of embodiments 2 to 15, wherein each R₂ is selected from N₃, CN, I and SCH₃.

Embodiment 28. The composition of embodiment 27, wherein R₂ is I.

Embodiment 29. The composition of any of embodiments 2 to 28, wherein each Y is O.

Embodiment 30. The composition of any of embodiments 2 to 28, wherein each Y is S.

Embodiment 31. The composition of any of embodiments 2 to 28, wherein each Y is CJ₄J₅.

Embodiment 32. The composition of any of embodiments 2 to 28, wherein each Y is CH₂.

Embodiment 33. The composition of any of embodiments 2 to 28, wherein each Y is NJ₆.

Embodiment 34. The composition of any of embodiments 2 to 28, wherein each Y is NH.

Embodiment 35. The composition of any of embodiments 2 to 28, wherein each Y is N(CH₃).

Embodiment 36. The composition of any of embodiments 2 to 28, wherein each Y is N(J₆)C(═O).

Embodiment 37. The composition of any of embodiments 2 to 28, wherein each Y is N(H)C(═O).

Embodiment 38. The composition of any of embodiments 2 to 28, wherein each Y is N(CH₃)C(═O).

Embodiment 39. The composition of any of embodiments 2 to 38, wherein each moiety of Formula I has the configuration:

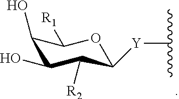

Embodiment 40. The composition of any of embodiments 2 to 38, wherein each moiety of Formula I has the configuration:

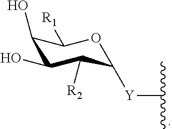

Embodiment 41. The composition of any of embodiments 2-40, wherein when Y is O and R₁ is CH₂OH then R₂ is other than OH, NH₂, and N(H)C(═O)CH₃.

Embodiment 42. The composition of any of embodiments 1 to 41, wherein the oligomeric compound comprises at least one modified nucleoside.

Embodiment 43 The composition of embodiment 42, wherein the at least one modified nucleoside comprises a modified base.

Embodiment 44. The composition of embodiment 42 or 43, wherein the at least one modified nucleoside comprises at least one modified sugar moiety.

Embodiment 45. The composition of embodiment 44, wherein at least one modified sugar moiety is a sugar surrogate.

Embodiment 46. The composition of embodiment 45, wherein the sugar surrogate is a tetrahydropyran.

Embodiment 47. The composition of any of embodiment 46, wherein the tetrahydropyran is F-HNA.

Embodiment 48. The composition of embodiment 45, wherein the sugar surrogate is a morpholino.

Embodiment 49. The composition of embodiment 1-48 wherein the oligomeric compound comprises at least one modified nucleoside comprising a modified sugar moiety selected from a bicyclic nucleoside and a 2′-modified nucleoside.

Embodiment 50. The composition of embodiment 49, wherein the oligomeric compound comprises at least one bicyclic nucleoside.

Embodiment 51. The composition of embodiment 50, wherein the bicyclic nucleoside is a (4′-CH₂—O-2′) BNA nucleoside.

Embodiment 52. The composition of embodiment 50, wherein the bicyclic nucleoside is a (4′-(CH₂)₂-0-2′) BNA nucleoside.

Embodiment 53. The composition of embodiment 50, wherein the bicyclic nucleoside is a (4′-C(CH₃)H—O-2′) BNA nucleoside.

Embodiment 54. The composition of embodiment 49-53, wherein the oligomeric compound comprises at least one 2′-modified nucleoside.

Embodiment 55. The composition of embodiment 54, wherein the oligomeric compound comprises at least one 2′-modified nucleoside selected from a 2′-F nucleoside, a 2′-OCH₃ nucleoside, and a 2′-O(CH₂)₂OCH₃ nucleoside.

Embodiment 56. The composition of embodiment 54, wherein at least one 2′-modified nucleoside is a 2′-F nucleoside.

Embodiment 57. The composition of embodiment 54, wherein at least one 2′-modified nucleoside is a 2′-OCH₃ nucleoside.

Embodiment 58. The composition of embodiment 54, wherein at least one 2′-modified nucleoside is a 2′-O(CH₂)₂OCH₃ nucleoside.

Embodiment 59. The composition of any of embodiments 1 to 58, wherein the oligomeric compound comprises at least one unmodified nucleoside.

Embodiment 60. The composition of embodiment 59, wherein the at least one unmodified nucleoside is a ribonucleoside.

Embodiment 61. The composition of embodiment 59, wherein the at least one unmodified nucleoside is a deoxyribonucleoside.

Embodiment 62. The composition of any of embodiments 1 to 61, wherein the oligomeric compound comprises at least two modified nucleosides.

Embodiment 63. The composition of embodiment 62, wherein the at least two modified nucleosides comprise the same modification.

Embodiment 64. The composition of embodiment 62, wherein the at least two modified nucleosides comprise different modifications.

Embodiment 65. The composition of any of embodiments 61 to 64, wherein at least one of the at least two modified nucleosides comprises a 2′-modification.

Embodiment 66. The composition of embodiment 65, wherein each of the at least two modified nucleosides is independently selected from 2′-F nucleosides, 2′-OCH₃ nucleosides and 2′-O(CH₂)₂OCH₃ nucleosides.

Embodiment 67. The composition of embodiment 66, wherein each of the at least two modified nucleosides is a 2′-F nucleoside.

Embodiment 68. The composition of embodiment 66, wherein each of the at least two modified nucleosides is a 2′-OCH₃ nucleosides.

Embodiment 69. The composition of embodiment 66, wherein each of the at least two modified nucleosides is a 2′-O(CH₂)₂OCH₃ nucleoside.

Embodiment 70. The composition of any of embodiments 1 to 69, wherein essentially every nucleoside of the oligomeric compound is a modified nucleoside.

Embodiment 71. The composition of any of embodiments 1 to 57 an 61 to 70, wherein every nucleoside of the oligomeric compound is a modified nucleoside.

Embodiment 72. The composition of any of embodiments 1 to 71, wherein the oligomeric compound is single-stranded.

Embodiment 73. The composition of any of embodiments 1 to 71, wherein the oligomeric compound is double-stranded.

Embodiment 74. The composition of any of embodiments 1 to 71, wherein the oligomeric compound is an antisense compound.

Embodiment 75. The composition of any of embodiments 1 to 71, wherein the oligomeric compound is a RISC based oligomeric compound.

Embodiment 76. The composition of any of embodiments 1 to 71, wherein the oligomeric compound is an siRNA duplex and the conjugate group is attached to the sense strand of the siRNA.

Embodiment 77. The composition of any of embodiments 1 to 71, wherein the oligomeric compound is an RNase H based antisense compound.

Embodiment 78. The composition of any of embodiments 1 to 77, wherein the conjugate group is attached to the 5′-terminal nucleoside of the oligomeric compound.

Embodiment 79. The composition of any of embodiments 1 to 77, wherein the conjugate group is attached to the 3′-terminal nucleoside of the oligomeric compound.

Embodiment 80. The composition of any of embodiments 1 to 77, wherein the conjugate group is attached to an internal nucleoside of the oligomeric compound.

Embodiment 81. The composition of any of embodiments 1 to 77, wherein the conjugate group increases uptake of the oligomeric compound into a hepatocyte relative to an unconjugated oligomeric compound.

Embodiment 82. The composition of any of embodiments 1 to 77, wherein the conjugate group increases the affinity of the oligomeric compound for a liver cell relative to an unconjugated oligomeric compound.

Embodiment 83. The composition of any of embodiments 1 to 77, wherein the conjugate group increases accumulation of the oligomeric compound in the liver relative to an unconjugated oligomeric compound.

Embodiment 84. The composition of any of embodiments 1 to 77, wherein the conjugate group decreases accumulation of the oligomeric compound in the kidneys relative to an unconjugated oligomeric compound.

Embodiment 85. The composition of embodiment 1 to 69 or 72 to 84, wherein the oligomeric compound has a sugar motif comprising:
a 5′-region consisting of 2-8 linked 5′-region nucleosides, wherein at least two 5′-region nucleosides are modified nucleosides and wherein the 3′-most 5′-region nucleoside is a modified nucleoside;
a 3′-region consisting of 2-8 linked 3′-region nucleosides, wherein at least two 3′-region nucleosides are modified nucleosides and wherein the 5′-most 3′-region nucleoside is a modified nucleoside; and
a central region between the 5′-region and the 3′-region consisting of 5-10 linked central region nucleosides, each independently selected from among: a modified nucleoside and an unmodified deoxynucleoside, wherein the 5′-most central region nucleoside is an unmodified deoxynucleoside and the 3′-most central region nucleoside is an unmodified deoxynucleoside.

Embodiment 86. The composition of embodiment 85, wherein the 5′-region consists of 2 linked 5′-region nucleosides.

Embodiment 87. The composition of embodiment 85, wherein the 5′-region consists of 3 linked 5′-region nucleosides.

Embodiment 88. The composition of embodiment 85, wherein the 5′-region consists of 4 linked 5′-region nucleosides.

Embodiment 89. The composition of embodiment 85, wherein the 5′-region consists of 5 linked 5′-region nucleosides.

Embodiment 90. The composition of any of embodiments 85 to 89, wherein the 3′-region consists of 2 linked 3′-region nucleosides.

Embodiment 91. The composition of any of embodiments 85 to 89, wherein the 3′-region consists of 3 linked 3′-region nucleosides.

Embodiment 92. The composition of any of embodiments 85 to 89, wherein the 3′-region consists of 4 linked 3′-region nucleosides.

Embodiment 93. The composition of any of embodiments 85 to 89, wherein the 3′-region consists of 5 linked 3′-region nucleosides.

Embodiment 94. The composition of any of embodiments 85 to 93, wherein the central region consists of 5 linked central region nucleosides.

Embodiment 95. The composition of any of embodiments 85 to 93, wherein the central region consists of 6 linked central region nucleosides.

Embodiment 96. The composition of any of embodiments 85 to 93, wherein the central region consists of 7 linked central region nucleosides.

Embodiment 97. The composition of any of embodiments 85 to 93, wherein the central region consists of 8 linked central region nucleosides.

Embodiment 98. The composition of any of embodiments 85 to 93, wherein the central region consists of 9 linked central region nucleosides.

Embodiment 99. The composition of any of embodiments 85 to 93, wherein the central region consists of 10 linked central region nucleosides.

Embodiment 100. The composition of any of embodiments 85 to 99, wherein the oligomeric compound consists of 14 to 26 linked nucleosides.

Embodiment 101. The composition of any of embodiments 85 to 99, wherein the oligomeric compound consists of 15 to 25 linked nucleosides.

Embodiment 102. The composition of any of embodiments 85 to 99, wherein the oligomeric compound consists of 16 to 20 linked nucleosides.

Embodiment 103. The composition of any of embodiments 85 to 102, wherein each modified nucleoside independently comprises a 2′-substituted sugar moiety or a bicyclic sugar moiety.

Embodiment 104. The composition of embodiment 103, wherein at least one modified nucleoside of the oligomeric compound comprises a 2′-substituted sugar moiety.

Embodiment 105. The composition of embodiment 104, wherein each modified nucleoside comprising a 2′-substituted sugar moiety comprises a 2′ substituent independently selected from among: halogen, optionally substituted allyl, optionally substituted amino, azido, optionally substituted SH, CN, OCN, CF₃, OCF₃, O, S, or N(Rₘ)-alkyl; O, S, or N(Rₘ)-alkenyl; O, S or N(Rₘ)-alkynyl; optionally substituted O-alkylenyl-O-alkyl, optionally substituted alkynyl, optionally substituted alkaryl, optionally substituted aralkyl, optionally substituted O-alkaryl, optionally substituted O-aralkyl, O(CH₂)₂SCH₃, O—(CH₂)₂—O—N(Rₘ)(Rₙ) or O—CH₂—C(═O)—N(Rₘ)(Rₙ), where each Rₘ and Rₙ is, independently, H, an amino protecting group or substituted or unsubstituted C₁-C₁₀ alkyl;

wherein each optionally substituted group is optionally substituted with a substituent group independently selected from among: hydroxyl, amino, alkoxy, carboxy, benzyl, phenyl, nitro (NO₂), thiol, thioalkoxy (S-alkyl), halogen, alkyl, aryl, alkenyl and alkynyl.

Embodiment 106. The composition of embodiment 104, wherein each 2′ substituent is independently selected from among: a halogen, OCH₃, OCH₂F, OCHF₂, OCF₃, OCH₂CH₃, O(CH₂)₂F, OCH₂CHF₂, OCH₂CF₃, OCH₂—CH═CH₂, O(CH₂)₂—OCH₃, O(CH₂)₂—SCH₃, O(CH₂)₂—OCF₃, O(CH₂)₃—N(R₃)(R₄), O(CH₂)₂—ON(R₃)(R₄), O(CH₂)₂—O(CH₂)₂—N(R₃)(R₄), OCH₂C(═O)—N(R₃)(R₄), OCH₂C(═O)—N(R₅)—(CH₂)₂—N(R₃)(R₄), and O(CH₂)₂—N(R₅)—C(═NR₆)[N(R₃)(R₄)]; wherein R₃, R₄, R₅ and R₆ are each, independently, H or C₁-C₆ alkyl.

Embodiment 107. The composition of embodiment 104, wherein each 2′ substituent is independently selected from among: a halogen, OCH₃, OCF₃, OCH₂CH₃, OCH₂CF₃, OCH₂—CH═CH₂, O(CH₂)₂—OCH₃ (MOE), O(CH₂)₂—O(CH₂)₂—N(CH₃)₂, OCH₂C(═O)—N(H)CH₃, OCH₂C(═O)—N(H)—(CH₂)₂—N(CH₃)₂, and OCH₂—N(H)—C(═NH)NH₂.

Embodiment 108. The composition of embodiment 104, wherein the at least one 2′-modified nucleoside comprises a 2′-MOE sugar moiety.

Embodiment 109. The composition of embodiment 104, wherein the at least one 2′-modified nucleoside comprises a 2′-OMe sugar moiety.

Embodiment 110. The composition of embodiment 104, wherein the at least one 2′-modified nucleoside comprises a 2′-F sugar moiety.

Embodiment 111. The composition of any of embodiments 85 to 110, wherein the oligomeric compound comprises at least one modified nucleoside comprising a sugar surrogate.

Embodiment 112. The composition of embodiment 111, wherein the modified nucleoside comprises an F-HNA sugar moiety.

Embodiment 113. The composition of embodiment 111, wherein the modified nucleoside comprises an HNA sugar moiety.

Embodiment 114. The composition of embodiment 111, wherein the modified nucleoside comprises a morpholino.

Embodiment 115. The composition of any of embodiments 85 to 114 wherein the oligomeric compound comprises at least one modified nucleoside comprising a bicyclic sugar moiety.

Embodiment 116. The composition of embodiment 115, wherein the bicyclic sugar moiety is a cEt sugar moiety.

Embodiment 117. The composition of embodiment 115, wherein bicyclic sugar moiety is an LNA sugar moiety.

Embodiment 118. The composition of any of embodiments 1 to 117, wherein the oligomeric compound comprises at least one modified internucleoside linkage.

Embodiment 119. The composition of any of embodiments 1 to 117, wherein each internucleoside linkage of the oligomeric compound is a modified internucleoside linkage.

Embodiment 120. The composition of any of embodiments 1 to 118, wherein the oligomeric compound comprises at least one unmodified phosphodiester internucleoside linkage.

Embodiment 121. The composition of any of embodiments 118 to 120, wherein at least one modified internucleoside linkage is a phosphosphorothioate internucleoside linkage.

Embodiment 122. The composition of any of embodiments 119 to 121, wherein each modified internucleoside linkage is a phosphorothioate internucleoside linkage.

Embodiment 123. The composition of any of embodiments 118 and 120 to 122, wherein the oligomeric compound comprises at least 2 phosphodiester internucleoside linkages.

Embodiment 124. The composition of any of embodiments 118 and 120 to 122, wherein the oligomeric compound comprises at least 3 phosphodiester internucleoside linkages.

Embodiment 125. The composition of any of embodiments 118 and 120 to 122, wherein the oligomeric compound comprises at least 4 phosphodiester internucleoside linkages.

Embodiment 126. The composition of any of embodiments 118 and 120 to 122, wherein the oligomeric compound comprises at least 5 phosphodiester internucleoside linkages.

Embodiment 127. The composition of any of embodiments 118 and 120 to 122, wherein the oligomeric compound comprises at least 6 phosphodiester internucleoside linkages.

Embodiment 128. The composition of any of embodiments 118 and 120 to 122, wherein the oligomeric compound comprises at least 7 phosphodiester internucleoside linkages.

Embodiment 129. The composition of any of embodiments 118 and 120 to 122, wherein the oligomeric compound comprises at least 8 phosphodiester internucleoside linkages.

Embodiment 130. The composition of any of embodiments 118 and 120 to 122, wherein the oligomeric compound comprises at least 9 phosphodiester internucleoside linkages.

Embodiment 131. The composition of any of embodiments 118 and 120 to 122, wherein the oligomeric compound comprises at least 10 phosphodiester internucleoside linkages.

Embodiment 132. The composition of any of embodiments 118 and 120 to 131, wherein the oligomeric compound comprises fewer than 16 phosphorothioate internucleoside linkages.

Embodiment 133. The composition of any of embodiments 118 and 120 to 131, wherein the oligomeric compound comprises fewer than 15 phosphorothioate internucleoside linkages.

Embodiment 134. The composition of any of embodiments 118 and 120 to 131, wherein the oligomeric compound comprises fewer than 14 phosphorothioate internucleoside linkages.

Embodiment 135. The composition of any of embodiments 118 and 120 to 131, wherein the oligomeric compound comprises fewer than 13 phosphorothioate internucleoside linkages.

Embodiment 136. The composition of any of embodiments 118 and 120 to 131, wherein the oligomeric compound comprises fewer than 12 phosphorothioate internucleoside linkages.

Embodiment 137. The composition of any of embodiments 118 and 120 to 131, wherein the oligomeric compound comprises fewer than 11 phosphorothioate internucleoside linkages.

Embodiment 138. The composition of any of embodiments 118 and 120 to 131, wherein the oligomeric compound comprises fewer than 10 phosphorothioate internucleoside linkages.

Embodiment 139. The composition of any of embodiments 118 and 120 to 131, wherein the oligomeric compound comprises fewer than 9 phosphorothioate internucleoside linkages.

Embodiment 140. The composition of any of embodiments 118 and 120 to 131, wherein the oligomeric compound comprises fewer than 8 phosphorothioate internucleoside linkages.

Embodiment 141. The composition of any of embodiments 118 and 120 to 131, wherein the oligomeric compound comprises fewer than 7 phosphorothioate internucleoside linkages.

Embodiment 142. The composition of any of embodiments 118 and 120 to 131, wherein the oligomeric compound comprises fewer than 6 phosphorothioate internucleoside linkages.

Embodiment 143. The composition of any of embodiments 1 to 142, wherein each terminal internucleoside linkage of the oligomeric compound is a phosphorothioate internucleoside linkage.

Embodiment 144. The composition of any of embodiments 1 to 129 or 132 to 143, wherein each internucleoside linkage linking two deoxynucleosides of the oligomeric compound is a phosphorothioate internucleoside linkage.

Embodiment 145. The composition of any of embodiments 1 to 129 or 132 to 143, wherein each non-terminal internucleoside linkage linking two modified nucleosides of the oligomeric compound is a phosphodiester internucleoside linkage.

Embodiment 146. The composition of any of embodiments 1 to 129 or 132 to 145, wherein each non-terminal internucleoside linkage of the oligomeric compound that is 3′ of a modified nucleoside is a phosphodiester internucleoside linkage.

Embodiment 147. The composition of any of embodiments 1 to 129 or 132 to 146, wherein each internucleoside linkage of the oligomeric compound that is 3′ of a deoxynucleoside is a phosphorothioate internucleoside linkage.

Embodiment 148. The composition of any of embodiments 1 to 129 or 132 to 147, wherein the oligomeric compound has a chemical motif selected from among:
MsMy(Ds)0-1(DsDs)(3-5)MsM
MsMy(Ds)0-1(DsDs)(3-5)MyMsM
MsMy(Ds)0-1(DsDs)(3-5)MyMyMsM
MsMy(Ds)0-1(DsDs)(3-5)MyMyMyMsM
MsMyMy(Ds)0-1(DsDs)(3-5)MsM
MsMyMy(Ds)0-1(DsDs)(3-5)MyMsM
MsMyMy(Ds)0-1(DsDs)(3-5)MyMyMsM
MsMyMy(Ds)0-1(DsDs)(3-5)MyMyMyMsM
MsMyMyMy(Ds)0-1(DsDs)(3-5)MsM
MsMyMyMy(Ds)0-1(DsDs)(3-5)MyMsM
MsMyMyMy(Ds)0-1(DsDs)(3-5)MyMyMsM
MsMyMyMy(Ds)0-1(DsDs)(3-5)MyMyMyMsM
MsMyMyMyMy(Ds)0-1(DsDs)(3-5)MsM
MsMyMyMyMy(Ds)0-1(DsDs)(3-5)MyMsM
MsMyMyMyMy(Ds)0-1(DsDs)(3-5)MyMyMsM; and
MsMyMyMyMy(Ds)0-1(DsDs)(3-5)MyMyMyMsM;
   wherein each M is independently a modified nucleoside, each D is a deoxynucleoside; each s is a phosphorothioate internucleoside linkage, and each y is either a phosphodiester internucleoside linkage or a phosphoro¬thioate internucleoside linkage, provided that at least one y is a phosphodiester internucleotide linkage.

Embodiment 149. The composition of any of embodiments 1 to 129 or 132 to 147, wherein the oligomers has a chemical motif selected from among:
MsMo(Ds)0-1(DsDs)(3-5)MoMsM
MsMo(Ds)0-1(DsDs)(3-5)MoMoMsM
MsMo(Ds)0-1(DsDs)(3-5)MoMoMoMsM
MsMoMo(Ds)0-1(DsDs)(3-5)MsM
MsMoMo(Ds)0-1(DsDs)(3-5)MoMsM
MsMoMo(Ds)0-1(DsDs)(3-5)MoMoMsM
MsMoMo(Ds)0-1(DsDs)(3-5)MoMoMoMsM
MsMoMoMo(Ds)0-1(DsDs)(3-5)MsM
MsMoMoMo(Ds)0-1(DsDs)(3-5)MoMsM
MsMoMoMo(Ds)0-1(DsDs)(3-5)MoMoMsM
MsMoMoMo(Ds)0-1(DsDs)(3-5)MoMoMoMsM
MsMoMoMoMo(Ds)0-1(DsDs)(3-5)MsM
MsMoMoMoMo(Ds)0-1(DsDs)(3-5)MoMsM
MsMoMoMoMo(Ds)0-1(DsDs)(3-5)MoMoMsM; and
MsMoMoMoMo(Ds)0-1(DsDs)(3-5)MoMoMoMsM;
   wherein each M is independently a modified nucleoside, each D is a deoxynucleoside; each o is a phosphodiester internucleoside linkage, and each s is a phosphoro¬thioate internucleoside linkage.

Embodiment 150. The composition of embodiment 148 or 149, wherein each M is independently selected from among a 2′substituted sugar moiety or a bicyclic nucleoside.

Embodiment 151. The composition of embodiment 150 wherein each M is independently selected from among: a 2′-MOE nucleoside and a bicyclic nucleoside.

Embodiment 152. The composition of embodiment 150 or 151, wherein each M is a 2′-MOE nucleoside.

Embodiment 153. The composition of embodiment 150 or 151, wherein each M is a cEt nucleoside.

Embodiment 154. The composition of embodiments 150 or 151, wherein each M is an LNA nucleoside.

Embodiment 155. The composition of any of embodiments 1 to 154, wherein the excipient is an excipient for oral administration that improves the oral delivery the composition.

Embodiment 156. The composition of any of embodiments 1 to 155, wherein the excipient comprises at least one penetration enhancer.

Embodiment 157. The composition of embodiment 156 wherein the penetration enhancer is selected from a fatty acid, bile acid, chelating agent and non-chelating non-surfactant.

Embodiment 158. The composition of embodiment 157 wherein the fatty acid is selected from arachidonic acid, oleic acid, lauric acid, capric acid, caprylic acid, myristic acid, palmitic acid, stearic acid, linoleic acid, linolenic acid, dicaprate, tricaprate, monoolein, dilaurin, glyceryl 1-monocaprate, 1-dodecyl-azacycloheptan-2-one, an acylcarnitine, an acylcholine and a monoglyceride or a pharmaceutically acceptable salt thereof.

Embodiment 159. The composition of embodiment 157 wherein the bile acid is selected from cholic acid, dehydrocholic acid, deoxycholic acid, glucholic acid, glycholic acid, glycodeoxycholic acid, taurocholic acid, taurodeoxycholic acid, chenodeoxycholic acid, ursodeoxycholic acid, sodium tauro-24, 25-dihydrof&sidate, sodium glycodihydrofusidate and polyoxyethylene-9-lauryl ether or a pharmaceutically acceptable salt thereof.

Embodiment 160. The composition of embodiment 157 wherein the chelating agent is selected from EDTA, citric acid, a salicylate, an N-acyl derivative of collagen, laureth-9 and an N-amino acyl derivative of a beta-diketone or a mixture thereof.

Embodiment 161. The composition of embodiment 157 wherein the non-chelating non-surfactant is selected from the group consisting of an unsaturated cyclic urea, 1-alkyl-alkanone, 1-alkenylazacycloalkanone a steroid anti-inflammatory agent or a mixture thereof.

Embodiment 162. The composition of embodiment 156 wherein the penetration enhancer comprises sodium caprate (C10) and/or sodium caprylate (C12).

Embodiment 163. The composition of any of embodiments 1 to 162 wherein the composition is a capsule, tablet, compression coated tablet or bilayer tablet.

Embodiment 164. The composition of embodiment 164 wherein the capsule, tablet, compression coated tablet or bilayer tablet comprises an enteric coating.

Embodiment 165. The composition of any of embodiments 1 to 164 wherein the composition comprises an enteric coating.

166. The composition of any of embodiments 1 to 165 wherein the excipient comprises a substance selected from poly-amino acids, polyimines, polyacrylates, polyalkylacrylates, polyoxethanes, polyalkylcyanoacrylatc, cationized gelatins, albumins, starches, acrylates, polyethylene glycol, DEAE-derivatized polyimines, pollulans and celluloses.

Embodiment 167. The composition of any of embodiments 1 to 166 wherein the excipient comprises a substance selected from chitosan, poly-L-lysine, polyhistidine, polyornithine, polyspermines, protamine, polyvinylpyridine, polythiodiethylamino-methylene P(TDAE), polyaminostyrene, poly(methylcyanoacrylate), poly(ethylcyanoacrylate), poly(butylcyanoacrylate), poly(isobutylcyanoacrylate), poly(isohexylcyanoacrylatc), DEAE-methacrylate, DEAE-ethylhexylacrylate, DEAE-acrylamide, DEAE-albumin, DEAE-dextran, polymethylacrylate, polyhexylacrylate, poly (D,L-lactic acid), poly (D,L-lactic-coglycolic acid) (PLGA) and polyethylene glycol (PEG).

Embodiment 168. The composition of any of embodiments 1 to 167 wherein the excipient comprises a substance selected from a complex of poty-L-lysine and alginate, a complex of protamine and alginate, lysine, dilysine, trilysine, calcium, glucosamine, arginine, galactosamine, nicotinamide, creatine, lysine-ethyl ester or arginine ethyl-ester.

Embodiment 169. The composition of any of embodiments 1 to 169 wherein the excipient comprises a substance selected from a delayed release coating or matrix selected from acetate phthalate, propylene glycol, sorbitan monoleate, cellulose acetate phthalate (CAP), cellulose acetate trimellitate, hydroxypropyl methyl cellulose phthalate (HPMCP), methacrylates, chitosan, guar gum and polyethylene glycol (PEG).

Embodiment 170. The composition of any of embodiments 1 to 169 wherein the excipient comprises a mucoadhesive patch.

Embodiment 171. The composition of any of embodiments 1 to 170, wherein the oligomeric compound has a nucleobase sequence comprising an at least 8 nucleobase portion complementary to an equal length portion of a target nucleic acid.

Embodiment 172. The composition of any of embodiments 1 to 170, wherein the oligomeric compound has a nucleobase sequence comprising an at least 10 nucleobase portion complementary to an equal length portion of a target nucleic acid.

Embodiment 173. The composition of any of embodiments 1 to 170, wherein the oligomeric compound has a nucleobase sequence comprising an at least 12 nucleobase portion complementary to an equal length portion of a target nucleic acid.

Embodiment 174. The composition of any of embodiments 1 to 170, wherein the oligomeric compound has a nucleobase sequence comprising an at least 14 nucleobase portion complementary to an equal length portion of a target nucleic acid.

Embodiment 175. The composition of any of embodiments 1 to 170, wherein the oligomeric compound has a nucleobase sequence comprising an at least 16 nucleobase portion complementary to an equal length portion of a target nucleic acid.

Embodiment 176. The composition of any of embodiments 1 to 170, wherein the oligomeric compound has a nucleobase sequence comprising an at least 18 nucleobase portion complementary to an equal length portion of a target nucleic acid.

Embodiment 177. The composition of any of embodiments 1 to 170, wherein the oligomeric compound is at least 90% complementary to a target nucleic acid.

Embodiment 178. The composition of any of embodiments 1 to 170, wherein the oligomeric compound is at least 95% complementary to a target nucleic acid.

Embodiment 179. The composition of any of embodiments 1 to 170, wherein the oligomeric compound is 100% complementary to a target nucleic acid.

Embodiment 180. The composition of any of embodiments 171 to 179, wherein the target nucleic acid is a pre-mRNA.

Embodiment 181. The composition of any of embodiments 171 to 179, wherein the target nucleic acid is an mRNA.

Embodiment 182. The composition of any of embodiments 171 to 179, wherein the target nucleic acid is a micro RNA.

Embodiment 183. The composition of any of embodiments 171 to 179, wherein the target nucleic acid is expressed in the liver.

Embodiment 184. The composition of any of embodiments 171 to 179, wherein the target nucleic acid is expressed in hepatocytes.

Embodiment 185. The composition of any of embodiments 171 to 179, wherein the target nucleic acid has the nucleobase sequence of any one of SEQ ID NOs.: 1, 2, 3, 4, 5, 6, 7, 8, 9, 10, 11, 12, 13, 14, 15, 16, 17, 18, 19, 20, 21, 22, 23, 24, or 82.

Embodiment 186. The composition of any of embodiments 169 to 177, wherein the target nucleic encodes a protein selected from among: Alpha 1 antitrypsin, Androgen Receptor, Apolipoprotein (a), Apolipoprotein B, Apolipoprotein C-III, C-Reactive Protein, eIF-4E, Factor VII, Factor XI, Glucocorticoid Receptor, Glucagon Receptor, HBV, Protein Tyrosine Phosphatase 1B, STAT3, SRB-1, Transthyretin, PCSK9, angiopoietin-like 3, plasma prekallikrein, and growth hormone receptor.

Embodiment 187. The composition of any of embodiments 171 to 181 wherein the target nucleic acid is a viral nucleic acid.

Embodiment 188. The composition of embodiment 187, wherein the viral nucleic acid expressed in the liver.

Embodiment 189. The composition of embodiment 186, wherein the target nucleic acid is a Hepatitis B viral nucleic acid.

Embodiment 190. The composition of embodiment 186, wherein the target nucleic acid is a HCV viral nucleic acid.

Embodiment 191. The composition of any of embodiments 1 to 181, wherein the oligomeric compound comprises the nucleobase sequence of any one of SEQ ID NOs.: 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, or 146.

Embodiment 192. The composition of any of embodiments 1 to 181, wherein the oligomeric compound comprises the nucleobase sequence of any one of SEQ ID NO.: 25, 26, 27, 28, 29, or 30.

Embodiment 193. The composition of any of embodiments 1 to 181, wherein the oligomeric compound comprises the nucleobase sequence of SEQ ID NO.: 31.

Embodiment 194. The composition of any of embodiments 1 to 181, wherein the oligomeric compound comprises the nucleobase sequence of SEQ ID NO.: 32.

Embodiment 195. The composition of any of embodiments 1 to 181, wherein the oligomeric compound comprises the nucleobase sequence of SEQ ID NO.: 33.

Embodiment 196. The composition of any of embodiments 1 to 181, wherein the oligomeric compound comprises the nucleobase sequence of SEQ ID NO.: 34.

Embodiment 197. The composition of any of embodiments 1 to 181, wherein the oligomeric compound comprises the nucleobase sequence of any of SEQ ID NOs.: 35, 36, 37, 38, 39, 40, 41, 42, or 43.

Embodiment 198. The composition of any of embodiments 1 to 181, wherein the oligomeric compound comprises the nucleobase sequence of SEQ ID NO.: 44, 45, 46, 47, or 48.

Embodiment 199. The composition of any of embodiments 1 to 181, wherein the oligomeric compound comprises the nucleobase sequence of any of SEQ ID NOs.: 49, 50, 51, 52, 53, 54, 55, 56, 57, 58, or 59.

Embodiment 200. The composition of any of embodiments 1 to 181, wherein the oligomeric compound comprises the nucleobase sequence of any of SEQ ID NOs.: 60, 61, 62, 63, 64, 65, 66, or 67.

Embodiment 201. The composition of any of embodiments 1 to 181, wherein the oligomeric compound comprises the nucleobase sequence of any of SEQ ID NO.: 69, 70, 71, or 72.

Embodiment 202. The composition of any of embodiments 1 to 181, wherein the oligomeric compound comprises the nucleobase sequence of SEQ ID NO.: 73.

Embodiment 203. The composition of any of embodiments 1 to 181, wherein the oligomeric compound comprises the nucleobase sequence of any of SEQ ID NOs.: 74, 75, 76, 77, 78, 79, 80, or 81.

Embodiment 204. The composition of any of embodiments 1 to 181, wherein the oligomeric compound comprises the nucleobase sequence of SEQ ID NO.: 68.

Embodiment 205. The composition of any of embodiments 1 to 181, wherein the oligomeric compound comprises the nucleobase sequence of any of SEQ ID NOs.: 82-103, 111, or 113.

Embodiment 206. The composition of any of embodiments 1 to 205, wherein the oligomeric compound is an antisense oligomeric compound.

Embodiment 207. The composition of any of embodiments 1 to 206, wherein the conjugate group does not comprise PEG.

Embodiment 208. The composition of any of embodiments 1 to 206, wherein the connector group does not comprise PEG.

Embodiment 209. The composition of any of embodiments 1 to 206, wherein the linking group does not comprise PEG.

Embodiment 210. The composition of any of embodiments 1 to 209 for the treatment of a disease or condition.

Embodiment 211. A method of administering the composition of any of embodiments 1 to 210, to an animal.

Embodiment 212. A method of treating a metabolic disorder comprising administering the composition of any of embodiments 1 to 210, to a subject in need thereof.

Embodiment 213. A method of treating a cardiovascular disorder comprising administering the composition of any of embodiments 1 to 210, to a subject in need thereof.

Embodiment 214. The method of any of embodiments 211 to 213, wherein the administration is oral.

Embodiment 215. The method of any of embodiments 211 to 213, wherein the administration is by enema.

Embodiment 216. The method of any of embodiments 2011 to 215, wherein the conjugated oligomeric compound is at least 90% complementary to a target nucleic acid.

Embodiment 217. The method of any of embodiments 211 to 215, wherein the conjugated oligomeric compound is 100% complementary to a target nucleic acid.

Embodiment 218. The method of any of embodiments 170 to 184, wherein the target nucleic acid is expressed in the liver.

Embodiment 219. The method of any of embodiments 170 to 185, wherein the target nucleic acid is expressed in hepatocytes.

Embodiment 220. The method of any of embodiments 170 to 186, wherein the target nucleic encodes a protein selected from among: Androgen Receptor, Apolipoprotein (a), Apolipoprotein B, Apolipoprotein C-III, C-Reactive Protein, eIF-4E, Factor VII, Factor XI, Glucocorticoid Receptor, Glucagon Receptor, Protein Tyrosine Phosphatase 1B, STAT3, and Transthyretin.

Embodiment 221. A method of modulating splicing of a pre-mRNA target nucleic acid in a cell comprising contacting the cell with a conjugated antisense compound, wherein the conjugated antisense compound comprises a modified oligonucleotide and a conjugate; and wherein the conjugate comprises a GalNac; and thereby modulating splicing of the pre-mRNA target nucleic acid in the cell.

Embodiment 220. The method of embodiment 219, wherein the pre-mRNA target nucleic acid is expressed in a hepatocyte.

Embodiment 221. A compound comprising an oligomeric compound and a conjugate group, wherein the conjugate group comprises a moiety having Formula I:

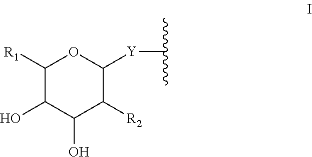

wherein:
R₁ is selected from Q₁, CH₂Q₁, CH₂OH, CH₂NJ₁J₂, CH₂N₃ and CH₂SJ₃;
Q₁ is selected from aryl, substituted aryl, heterocyclic, substituted heterocyclic, heteroaryl and substituted heteroaryl;
R₂ is selected from N₃, CN, halogen, N(H)C(═O)-Q₂, substituted thiol, aryl, substituted aryl, heterocyclic, substituted heterocyclic, heteroaryl and substituted heteroaryl;
Q₂ is selected from H, C₁-C₆ alkyl, substituted C₁-C₆ alkyl, C₁-C₆ alkoxy, substituted C₁-C₆ alkoxy, aryl, substituted aryl, heterocyclic, substituted heterocyclic, heteroaryl and substituted heteroaryl;
Y is selected from O, S, CJ₄J₅, NJ₆ and N(J₆)C(═O);
J₁, J₂, J₃, J₄, J₅, and J₆ are each, independently, H or a substituent group;
each substituent group is, independently, mono or poly substituted with optionally protected substituent groups independently selected from halogen, C₁-C₆ alkyl, C₁-C₆ alkoxy, aryl, heterocyclic and heteroaryl wherein each substituent group can include a linear or branched alkylene group optionally including one or more groups independently selected from O, S, NH and C(═O), and wherein each substituent group may be further substituted with one or more groups independently selected from C₁-C₆ alkyl, halogen or C₁-C₆ alkoxy wherein each cyclic group is mono or polycyclic; and when Y is O and R₁ is OH then R₂ is other than OH and N(H)C(═O)CH₃.

Embodiment 222. The compound of embodiment 221 wherein the moiety having Formula I is linked to the oligomeric compound through a connecting group.

Embodiment 223. The compound of embodiment 221 or 222 having Formula II:

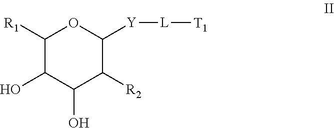

wherein:
R₁ is selected from Q₁, CH₂Q₁, CH₂OH, CH₂NJ₁J₂, CH₂N₃ and CH₂SJ₃;
Q₁ is selected from aryl, substituted aryl, heterocyclic, substituted heterocyclic, heteroaryl and substituted heteroaryl;
R₂ is selected from N₃, CN, halogen, N(H)C(═O)-Q₂, substituted thiol, aryl, substituted aryl, heterocyclic, substituted heterocyclic, heteroaryl and substituted heteroaryl;
Q₂ is selected from H, C₁-C₆ alkyl, substituted C₁-C₆ alkyl, C₁-C₆ alkoxy, substituted C₁-C₆ alkoxy, aryl, substituted aryl, heterocyclic, substituted heterocyclic, heteroaryl and substituted heteroaryl;
Y is selected from O, S, CJ₄J₅, NJ₆ and N(J₆)C(═O);
L is a connecting group;
J₁, J₂, J₃, J₄, J₅ and J₆ are each, independently, H or a substituent group;
T₁ is said oligomer; and
each substituent group is, independently, mono or poly substituted with optionally protected substituent groups independently selected from halogen, C₁-C₆ alkyl, C₁-C₆ alkoxy, aryl, heterocyclic and heteroaryl wherein each substituent group can include a linear or branched alkylene group optionally including one or more groups independently selected from O, S, NH and C(═O), and wherein each substituent group may be further substituted with one or more groups independently selected from C₁-C₆ alkyl, halogen or C₁-C₆ alkoxy wherein each cyclic group is mono or polycyclic.

In embodiments having more than one of a particular variable (e.g., more than one “m” or “n”), unless otherwise indicated, each such particular variable is selected independently. Thus, for a structure having more than one n, each n is selected independently, so they may or may not be the same as one another.

### DETAILED DESCRIPTION

It is to be understood that both the foregoing general description and the following detailed description are exemplary and explanatory only and are not restrictive of the disclosure. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The present disclosure provides compositions and methods for the local as well as systemic delivery of conjugated oligomeric compounds such as conjugated antisense compounds to an animal via non-parenteral means. In particular, the present invention provides compositions and methods for modulating the in vivo expression of a gene in an animal through the non-parenteral administration of a conjugated oligomeric compound, thereby circumventing the complications and expense which may be associated with intravenous and other parenteral routes of administration.

In certain embodiments, enhanced bioavailability is achieved via the non-parenteral administration of compositions provided herein. The term “bioavailability” refers to a measurement of what portion of an administered drug reaches the circulatory system when a non-parenteral mode of administration is used to introduce the drug into an animal. The term is used for drugs whose efficacy is related to the blood concentration achieved, even if the drug's ultimate site of action is intracellular (van Berge-Henegouwen et al., Gastroenterol., 1977, 73, 300). Traditionally, bioavailability studies determine the degree of intestinal absorption of a drug by measuring the change in peripheral blood levels of the drug after an oral dose (DiSanto, Chapter 76 In: Remington's Pharmaceutical Sciences, 18th Ed., Gennaro, ed., Mack Publishing Co., Easton, PA, 1990, pages 1451-1458). The area under the curve (AUC₀) is divided by the area under the curve after an intravenous (i.v.) dose (AUCᵢᵥ) and the quotient is used to calculate the fraction of drug absorbed. This approach cannot be used, however, with compounds which have a large “first pass clearance,” i.e., compounds for which hepatic uptake is so rapid that only a fraction of the absorbed material enters the peripheral blood. For such compounds, other methods must be used to determine the absolute bioavailability (van Berge-Henegouwen et al., Gastroenterol., 1977, 73, 300). With regards to oligonucleotides, studies suggest that they are rapidly eliminated from plasma and accumulate mainly in the liver and kidney after i.v. administration (Miyao et al., Antisense Res. Dev., 1995, 5, 115; Takakura et al., Antisense & Nucl. Acid Drug Dev., 1996, 6, 177).

Another “first pass effect” that applies to orally administered drugs is degradation due to the action of gastric acid and various digestive enzymes. Furthermore, the entry of many high molecular weight active agents (such as peptides, proteins and oligonucleotides) and some conventional and/or low molecular weight drugs (e.g., insulin, vasopressin, leucine enkephalin, etc.) through mucosal routes (such as oral, pulmonary, buccal, rectal, transdermal, vaginal and ocular) to the bloodstream is frequently obstructed by poor transport across epithelial cells and concurrent metabolism during transport. This type of degradative metabolism is known for oligonucleotides and nucleic acids. For example, phosphodiesterases are known to cleave the phosphodiester linkages of oligonucleotides and many other modified linkages present in oligomeric compounds such as synthesized oligonucleotides.

One means of ameliorating first pass clearance effects is to increase the dose of administered drug, thereby compensating for proportion of drug lost to first pass clearance. Although this may be readily achieved with i.v. administration by, for example, simply providing more of the drug to an animal, other factors influence the bioavailability of drugs administered via non-parenteral means. For example, a drug may be enzymatically or chemically degraded in the alimentary canal or blood stream and/or may be impermeable or semipermeable to various mucosal membranes.

It has now been found that oligonucleotides can be introduced effectively into animals via non-parenteral means through coadministration of “mucosal penetration enhancers,” also known as “absorption enhancers” or simply as “penetration enhancers”. These are substances which facilitate the transport of a drug across mucous membrane(s) associated with the desired mode of administration.

As used herein “excipient” means any compound or mixture of compounds that is added to a composition as provided herein that is suitable for non-parenteral delivery of a conjugated oligomeric compound. In certain embodiments, an excipient enhances the uptake of the conjugated oligomeric compound and or the ultimate bioavailability of the oligomeric compound. Typical pharmaceutical excipients include, but are not limited to, penetration enhancers, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, EXPLOTAB); and wetting agents (e.g., sodium lauryl sulphate, etc.). In certain embodiments, an excipient as used herein may include any compounds or mixture of compounds that improve oral delivery of the compositions of the present invention.

In certain embodiments, the excipient includes a pharmaceutically acceptable solvent, suspending agent or any other pharmacologically inert vehicle for delivering one or more conjugated oligomeric compounds to an animal. The excipient may be liquid or solid and is selected, with the planned manner of administration in mind, so as to provide for the desired bulk, consistency, etc., when combined with a conjugated oligomeric compound and the other components of a given pharmaceutical composition. Typical pharmaceutical carriers include, but are not limited to, binding agents (e.g., pregelatinised maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, EXPLOTAB); and wetting agents (e.g., sodium lauryl sulphate, etc.). Excipients may include one or more penetration enhancers, a capsule or pill formulation, an enteric component or any other pharmaceutically acceptable component.

In certain embodiments, the excipient includes a pharmaceutically acceptable organic or inorganic carrier substances suitable for oral administration which do not deleteriously react with nucleic acids can also be used to formulate the compositions of the present invention. Suitable pharmaceutically acceptable carriers include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone and the like. The formulations can be sterilized and, if desired, mixed with auxiliary agents, e.g., lubricants, preservatives, stabilizers, wetting agents, emulsifiers, salts for influencing osmotic pressure, buffers, colorings flavorings and/or aromatic substances and the like which do not deleteriously interact with the oligomeric compounds of the formulation.

In certain embodiments, the excipient includes emulsifiers, stabilizers, dyes, and anti-oxidants may also be present in emulsions as needed. Pharmaceutical emulsions may also be multiple emulsions that are comprised of more than two phases such as, for example, in the case of oil in water in oil (o/w/o) and water in oil in water (w/o/w) emulsions. Such complex formulations often provide certain advantages that simple binary emulsions do not. Multiple emulsions in which individual oil droplets of an o/w emulsion enclose small water droplets constitute a w/o/w emulsion. Likewise a system of oil droplets enclosed in globules of water stabilized in an oily continuous provides an o/w/o emulsion.

In certain embodiments, the excipient includes one or more penetration enhancers. Penetration enhancers have been widely studied as a means to increase both paracellular and transcellular uptake of compounds. There are at least 11 distinct chemical categories of penetration enhancers (Whitehead et al., Pharmaceutical Research, 2008, 25(8), 1782-1788). These categories include but are not limited to anionic surfactants, cationic surfactants, zwitterionic surfactants, nonionic surfactants, bile salts, fatty acids fatty esters, fatty amines, sodium salts of fatty acids, nitrogen containing rings, and others. In addition, bacterial toxins (enterotoxins) have also been shown to increase permeability of the gastrointestinal layer, as well as the process of inflammation itself (Salama, et al., Advanced Drug Delivery Reviews, 2006, 58, 15-28). Glucose solutions have been shown to expand tight junctions (Salamat-Miller, et al., International Journal of pharmaceutics, 2005, 294, 201-216); the data suggest that the effects of a meal initiates the opening of the tight junctions by osmotic force, stimulating the flow of water through the paracellular pathway and carrying dissolved solutes in the convective stream (the so called “solvent drag”). Peptide based permeability enhancers, including toxins and venom derivatives have also been used, and some are briefly described below. Finally, there are a host of technologies to encapsulate actives for transport, not all of which are targeted for class III molecules, marketed by companies for oral delivery of poorly absorbed compounds.

In certain embodiments, the excipient includes one or more penetration enhancers selected from sodium caprate (C10) alone or in mixture, sodium caprylate (C12) alone or in mixture, sodium-2-ocyldodecanoate (C20) alone or in mixture, UDCA alone or in mixture, fatty acid mixture (C10, C12, C20, and/or UDCA), SNAC and AT1006.

In certain embodiments, the excipient includes one or more penetration enhancers selected from sodium laurate, bile salts, PEG-3350, POE, lecithin, Gantrex-AN-169, 5% Gantrex.AN-169 and 5% Carbopol 974P, 1% Eudragit, Labrasol, alkyl saccharide, lipids, EDTA buffer, Gantrez/bioadhesives, sodium phosphate tribasic and UDC.

In certain embodiments, the excipient includes one or more compounds and or mixtures selected from Sodium Caprate (C10), either alone or in conjunction with Sodium Caprylate (C12); Transcellular N-[8-2-hydroxybenzoyl) aminol caprylate (an acetylated amino acid); C12, sodium caprylate as an adjunct to C10; UDCA, also used as an adjunct to C10; sodium laurate; bile salts, fatty acids mixture (C10, C12, UDCA); POE; Lecithin; C20 (sodium-2-ocyldodecanoate); PEG 3350; Gantrex AN-169; 5% Gantrex AN-169 and 5% Carbopol 974P; 5% Gantrex AN-169; 1% Eudragit; Cumulase, Labrasol; alkyl saccharide; lipids; EDTA; Gantrez with bioadhesives; sodium phosphate tribasic and UDC.

In certain embodiments, the excipient includes one or more compounds and or mixtures selected from Chitosans, biodegradable mucopolysaccharides; Zonal Occluden toxin (ZOT); Melittin; C-CPE; Cell Penetrating Peptides (CPPs); Proteases; Lipids (sphingosines, alkylglucosides, oxidized lipids, ether lipids); and Multiple tight junction targeted modulators have been tried and reviewed (Deli, Maria A., Biochimica et Biophysica Acta, 2009, 1788, 892-910).

In certain embodiments, the excipient includes Nano-particles and or other carriers, to ferry macromolecules across the membrane, either as complexes in lipid matrixes or other complex macromolecules.

In certain embodiments, the excipient includes a mucoadhesive patch system for drug delivery (PCT International application WO 03/007913 A2, published on Jan. 30, 2003, the entire contents of which are incorporated herein by reference).

A “pharmaceutically acceptable” component of a formulation of the invention is one which, when used together with excipients, diluents, stabilizers, preservatives and other ingredients are appropriate to the nature, composition and mode of administration of a formulation. Accordingly it is desired to select penetration enhancers which facilitate the uptake of conjugated oligomeric compounds such as conjugated antisense oligonucleotides, without interfering with the activity of the oligomeric compounds and in a manner such that the same can be introduced into the body of an animal without unacceptable side-effects such as toxicity, irritation or allergic response.

In certain embodiments, the present invention provides compositions comprising one or more pharmaceutically acceptable penetration enhancers, and methods of using such compositions, which result in the improved bioavailability of conjugated oligomeric compounds administered via non-parenteral modes of administration. Heretofore, certain penetration enhancers have been used to improve the bioavailability of certain drugs. See Muranishi, Crit. Rev. Ther. Drug Carrier Systems, 1990, 7, 1 and Lee et al., Crit. Rev. Ther. Drug Carrier Systems, 1991, 8, 91. However, it is generally viewed to be the case that effectiveness of such penetration enhancers is unpredictable. Therefore, it has been surprisingly found that the uptake and delivery of oligonucleotides, relatively complex molecules which are known to be difficult to administer to animals and man, can be greatly improved even when administered by non-parenteral means through the use of a number of different classes of penetration enhancers.

In certain embodiments, the present invention provides compositions comprising one or more carrier particles. As used herein “carrier particle” means a granule, bead, microparticle, miniparticle, nanoparticle or any other solid dosage form which can be incorporated into an oral pharmaceutical formulation as described herein.

Preferred carrier particle-forming substances include poly-amino acids, polyimines, polyacrylates, dendrimers, polyalkylcyanoacrylates, cationized gelatins, albumins, starches, acrylates, polyethyleneglycols (PEG), DEAE-derivatized polyimines, pollulans and celluloses.

In other preferred embodiments, the carrier particle-forming substance includes polycationic polymers such as chitosan, poly-L-lysine, polyhistidine, polyornithine, polyspermines, protamine, polyvinylpyridine, polythiodiethylamino-methylene P(TDAE), polyaminostyrene (e.g. para-amino), poly(methylcyanoacrylate), poly (ethylcyanoacrylate), poly (butylcyanoacrylate), poly(isobutylcyanoacrylate), poly(isohexylcyanoacrylate), DEAE-methacrylate, DEAE-hexylacrylate, DEAE-acrylamide, DEAE-albumin and DEAE-dextran. In another preferred embodiment, the particle-forming substance is poly-L-lysine complexed with alginate.

In certain embodiments, carrier particle-forming substances are non-polycationic, i.e., carry an overall neutral or negative charge, such as polyacrylates, for example polyalkylacrylates (e.g., methyl, hexyl), polyoxethanes, poly(DL-lactic-co-glycolic acid) (PLGA) and polyethyleneglycol.

In certain embodiments, the pharmaceutical compositions of the invention may further comprise a bioadhesive material that serves to adhere carrier particles to mucosal membranes. Carrier particles may themselves be bioadhesive, as is the case with PLL-alginate carrier particles, or may be coated with a bioadhesive material. Such materials are well known in the formulation art, examples of which are described in PCT WO85/02092, the contents of which are incorporated herein by reference in its entirety. Preferred bioadhesive materials include polyacrylic polymers (e.g. carbomer and derivatives of carbomer), tragacanth, polyethyleneoxide cellulose derivatives (e.g. methylcellulose, carboxymethylcellulose, hydroxypropylmethylcellulose (HPMC), hydroxyethylcellulose (HEC), hydroxypropylcellulose (HPC) and sodium carboxymethylcellulose (NaCPC)), karya gum, starch, gelatin and pectin.

The compositions of the invention may further comprise a mucolytic substance which serves to degrade or erode mucin, partially or completely, at the site of the mucosal membrane to be traversed. Mucolytic substances are well known in the formulation art and include N-acetylcysteine, dithiothreitol, pepsin, pilocarpine, guaifenesin, glycerol guaiacolate, terpin hydrate, ammonium chloride, guattenesin, ambroxol, bromhexine, carbocysteine, domiodol, letosteine, mecysteine, mesna, sobrerol, stepronin, tiopronin and tyloxapol.

In certain embodiments, conjugated oligomeric compounds are associated with the carrier particles by electrostatic (e.g., ionic, polar, Van der Waals), covalent or mechanical (non-electrostatic, non-covalent) interactions depending on the drug and carrier particles, as well as the method of preparing the carrier particles. For example, an anionic drug such as a conjugated oligomeric compound such as an antisense oligonucleotide can be bound to cationic carrier particles by ionic interaction.

The effective non-parenteral use and administration of compositions of the present invention involves consideration of a number of different aspects about drug therapy. One important consideration when using the compositions and methods of the present invention is the mode of administration of the pharmaceutical composition containing the therapeutic conjugated oligomeric compound such as a conjugated antisense oligonucleotide. Non-parenteral modes of administration include, but are not limited to, buccal, sublingual, endoscopic, oral, rectal, transdermal, topical, nasal, intratracheal, pulmonary, urethral, vaginal, and ocular. In certain embodiments, administered by such non-parenteral modes the methods and compositions of the present invention deliver drug both locally and systemically as desired.

Another consideration of importance when using the compositions and methods of the present invention is the use and nature of penetration enhancers and carriers. Penetration enhancers facilitate the transport of drug molecules, for example, oligonucleotides and other nucleic acids, across mucosal and other epithelial cell membranes. Penetration enhancers include, but are not limited to, members of molecular classes such as surfactants, fatty acids, bile salts, chelating agents, and non-chelating non-surfactant molecules. Carriers are inert molecules that may be included in the compositions of the present invention to interfere with processes that lead to reduction in the levels of bioavailable nucleic acid or oligonucleotide drug.

Another important aspect of the compositions and methods of the present invention is the nature of the conjugated oligomeric compounds used. The conjugated oligomeric compounds of the present invention may be modified by using various conjugate groups and modified oligomeric compounds. Such modified oligomeric compounds comprise at least one modified internucleoside linkage, modified sugar, modified base or any combination thereof.

In certain embodiments, the absorption of conjugated oligomeric compounds is enhanced through modification of the oligomeric compound (Geary, et al., The Journal of Pharmacology and Experimental Therapeutics, 2001, 296 (3), 898-904). Such modifications include but are not limited decreased length of the oligomeric compound, 2′-MOE substituent groups, 5′-methylation of cytosines, and the presence of phosphodiester backbone in MOE modified compounds. Such modifications have been shown to increased intestinal permeability of antisense compounds in rats using an in situ infusion model. Methylphosphonate linkages have been shown to reduce the charge on oligomeric compounds and thus increase permeability.

Another important aspect of the compositions and methods of the present invention is the nature of the composition. Pharmaceutical compositions of the present invention include, but are not limited to, solutions, emulsions (including microemulsions and creams), and liposome-containing formulations. These compositions may be generated from a variety of components that include, but are not limited to, preformed liquids, self-emulsifying solids and self-emulsifying semisolids. The compositions of the present invention may be formulated into any of many possible dosage forms such as, but not limited to, tablets, capsules, liquid syrups, soft gels, suppositories, and enemas.

In certain embodiments, the compositions of the invention are provided for oral administration in the form of a capsule, tablet, compression coated tablet or bilayer tablet. In certain embodiments, these formulations comprise an enteric outer coating which resists degradation in the stomach and dissolves in the intestinal lumen. In certain embodiments, the formulation comprises an enteric material effective in protecting the conjugated oligomeric compounds from pH extremes of the stomach, or in releasing the conjugated oligomeric compounds over time to optimize the delivery thereof to a particular mucosal site. Enteric materials for acid-resistant tablets, capsules and caplets are known in the art and typically include acetate phthalate, propylene glycol, sorbitan monoleate, cellulose acetate phthalate (CAP), cellulose acetate trimellitate, hydroxypropyl methyl cellulose phthalate (HPMCP), methacrylates, chitosan, guar gum, pectin, locust bean gum and polyethylene glycol (PEG). One particularly useful type of methacrylate are the Eudragits™. These are anionic polymers that are water-impermeable at low pH, but become ionized and dissolve at intestinal pH. EUDRAGITS™ L100 and S100 are copolymers of methacrylic acid and methyl methacrylate.

Enteric materials may be incorporated within the dosage form or may be a coating substantially covering the entire surface of tablets, capsules or caplets. Enteric materials may also be accompanied by plasticizers that impart flexible resiliency to the material for resisting fracturing, for example during tablet curing or aging. Plasticizers are known in the art and typically include diethyl phthalate (DEP), triacetin, dibutyl sebacate (DBS), dibutyl phthalate (DBP) and triethyl citrate (TEC).

Another important aspect of the compositions and methods of the present invention is the means by which such compositions may be administered. Thus the dose, method of administration or application, and the use of additives are all worthy of consideration in this regard. Further, the methods and compositions of the present invention may be used to ameliorate a variety of diseases via local or systemic treatment. Such local or systemic treatment may be accomplished using the methods and compositions of the present invention via modes of administration that include, but are not limited to, buccal, sublingual, endoscopic, oral, rectal, transdermal, topical, nasal, pulmonary, urethral, vaginal, and ocular modes.

The present invention provides compositions and methods for local and systemic delivery of one or more oligomeric compounds to an animal via non-parenteral administration. For purposes of the invention, the term “animal” is meant to encompass humans as well as other mammals, as well as reptiles, fish, amphibians, and birds. The term “non-parenteral delivery” refers to the administration, directly or otherwise, of the drug via a non-invasive procedure which typically does not entail the use of a syringe and needle. Non-parenteral administration may be, but is not limited to, delivery of the drug via the alimentary canal or via transdermal, topical, nasal, pulmonary, urethral, vaginal or ocular routes. The term “alimentary canal” refers to the tubular passage in an animal that functions in the digestion and absorption of food and the elimination of food residue, which runs from the mouth to the anus, and any and all of its portions or segments, e.g., the oral cavity, the esophagus, the stomach, the small and large intestines and the colon, as well as compound portions thereof such as, e.g., the gastro-intestinal tract. Thus, the term “alimentary delivery” encompasses several routes of administration including, but not limited to, oral, rectal, endoscopic and sublingual/buccal administration. A common requirement for these modes of administration is absorption over some portion or all of the alimentary tract and a need for efficient mucosal penetration of the nucleic acid(s) so administered.

In addition, iontophoresis (transfer of ionic solutes through biological membranes under the influence of an electric field) (Lee et al., Critical Reviews in Therapeutic Drug Carrier Systems, 1991, p. 163), phonophoresis or sonophoresis (use of ultrasound to enhance the absorption of various therapeutic agents across biological membranes, notably the skin and the cornea) (Lee et al., Critical Reviews in Therapeutic Drug Carrier Systems, 1991, p. 166), and optimization of vehicle characteristics relative to dose deposition and retention at the site of administration (Lee et al., Critical Reviews in Therapeutic Drug Carrier Systems, 1991, p. 168) may be useful methods for enhancing the transport of drugs across mucosal sites in accordance with the present invention.

Drugs administered by the oral route can often be alternatively administered by the lower enteral route, i.e., through the anus into the rectum or lower intestine. Rectal suppositories, retention enemas or rectal catheters can be used for this purpose and may be preferred when patient compliance might otherwise be difficult to achieve (e.g., in pediatric and geriatric applications, or when the patient is vomiting or unconscious). Rectal administration can result in more prompt and higher blood levels than the oral route. (Harvey, Chapter 35 In: Remington's Pharmaceutical Sciences, 18th Ed., Gennaro, ed., Mack Publishing Co., Easton, PA, 1990, page 711). Because about 50% of the drug that is absorbed from the rectum will bypass the liver, administration by this route significantly reduces the potential for first-pass metabolism (Benet et al., Chapter 1 In: Goodman & Gilman's The Pharmacological Basis of Therapeutics, 9th Ed., Hardman et al., eds., McGraw-Hill, New York, NY, 1996).

Endoscopy may be used for drug delivery directly to an interior portion of the alimentary tract. For example, endoscopic retrograde cystopancreatography (ERCP) takes advantage of extended gastroscopy and permits selective access to the biliary tract and the pancreatic duct (Hirahata et al., Gan To Kagaku Ryoho, 1992, 19(10 Suppl.), 1591). Pharmaceutical compositions, including liposomal formulations, can be delivered directly into portions of the alimentary canal, such as, e.g., the duodenum (Somogyi et al., Pharm. Res., 1995, 12, 149) or the gastric submucosa (Akamo et al., Japanese J Cancer Res., 1994, 85, 652) via endoscopic means. Gastric lavage devices (Inoue et al., Artif Organs, 1997, 21, 28) and percutaneous endoscopic feeding devices (Pennington et al., Ailment Pharmacol. Ther., 1995, 9, 471) can also be used for direct alimentary delivery of pharmaceutical compositions.

The preferred method of non-parenteral administration for most drugs is oral delivery. This is typically the most convenient route for access to the systemic circulation. Absorption from the alimentary canal is governed by factors that are generally applicable, e.g., surface area for absorption, blood flow to the site of absorption, the physical state of the drug and its concentration at the site of absorption (Benet et al., Chapter 1 In: Goodman & Gilman's The Pharmacological Basis of Therapeutics, 9th Ed., Hardman et al., eds., McGraw-Hill, New York, NY, 1996, pages 5-7). A significant factor which may limit the oral bioavailability of a drug is the degree of “first pass effects.” For example, some substances have such a rapid hepatic uptake that only a fraction of the material absorbed enters the peripheral blood (Van Berge-Henegouwen et al., Gastroenterology, 1977, 73:300). The compositions and methods of the invention circumvent, at least partially, such first pass effects by providing improved uptake of nucleic acids by, e.g., causing the hepatic uptake system to become saturated and allowing a significant portion of the nucleic acid so administered to reach the peripheral circulation.

Topical administration is often chosen when local delivery of a drug is desired at, or immediately adjacent to the point of application of the drug composition or formulation. Although occasionally enough drug is absorbed into the systemic circulation to cause systemic effects, topical routes generally are not effective for systemic therapy. Three general types of topical routes of administration are recognized, topical administration of a drug composition to mucous membranes, skin or eyes.

Drugs that are applied to the mucous membranes produce primarily local effects. This route of administration includes application of drug compositions to mucous membranes of the conjunctiva, nasopharynx, oropharynx, vagina, colon, urethra, and urinary bladder. Absorption of drugs occurs rapidly through mucous membranes and is an effective route for localized therapy and, on occasion, for systemic therapy.

Transdermal drug delivery is a valuable route for the administration of lipid soluble therapeutics. It has been recognized that the dermis is more permeable than the epidermis and therefore absorption of drugs is much more rapid through abraded, burned or denuded skin. Inflammation and other physiologic conditions that increase blood flow to the skin also enhance absorption via the transdermal route. Absorption by this route may be enhanced via the use of an oily vehicle (inunction) or through the use of penetration enhancers. Hydration of the skin and the use of controlled release topical patches are also effective ways to administer drugs via the transdermal route. This route provides a means to deliver the drug for both systemic and local therapy.

Ocular delivery of drugs is especially useful for the local treatment of eye infections or abnormalities. The drug is typically administered via instillation and absorption of the drug occurs through the cornea. Corneal infection or trauma may thus result in more rapid absorption. Opthalmic delivery systems that provide prolonged duration of action (e.g., suspensions and ointments) and ocular inserts that provide continuous delivery of low amounts of drugs are useful additions to ophthalmic therapy. The ocular delivery of drugs results in predominantly local effects. Systemic absorption that results from drainage via the nasolachrimal canal is limited and few systemic side effects are typically observed.

In certain embodiments, the compositions of the present invention comprise one or more penetration enhancers in order to effect transport of conjugated oligomeric compounds across mucosal and epithelial membranes. Penetration enhancers may be classified as belonging to one of five broad categories—surfactants, fatty acids, bile salts, chelating agents, and non-chelating non-surfactants (Lee et al., Critical Reviews in Therapeutic Drug Carrier Systems, 1991, p. 92). Each of these classes is discussed in more detail in the following paragraphs. Carrier substances (or simply “carriers”), which reduce first pass effects by, e.g., saturating the hepatic uptake system, are also herein described.

In connection with the present invention, surfactants (or “surface-active agents”) are chemical entities which, when dissolved in an aqueous solution, reduce the surface tension of the solution or the interfacial tension between the aqueous solution and another liquid, with the result that absorption of conjugated oligomeric compounds through the alimentary mucosa and other epithelial membranes is enhanced. In addition to bile salts and fatty acids, surfactants include, for example, sodium lauryl sulfate, polyoxyethylene-9-lauryl ether and polyoxyethylene-20-cetyl ether (Lee et al., Critical Reviews in Therapeutic Drug Carrier Systems, 1991, page 92); and perfluorohemical emulsions, such as FC-43 (Takahashi et al., J. Pharm. Phamacol., 1988, 40, 252).

In certain embodiments, one or more fatty acids including their derivatives which act as penetration enhancers are used in compositions of the present invention. Such fatty acids include, for example, oleic acid, lauric acid, capric acid (n-decanoic acid), myristic acid, palmitic acid, stearic acid, linoleic acid, linolenic acid, dicaprate, tricaprate, monoolein (1-monooleoyl-rac-glycerol), dilaurin, caprylic acid, arachidonic acid, glyceryl 1-monocaprate, 1-dodecylazacycloheptan-2-one, acylcarnitines, acylcholines and mono- and di-glycerides thereof and/or physiologically acceptable salts thereof (i.e., oleate, laurate, caprate, myristate, palmitate, stearate, linoleate, etc.) (Lee et al., Critical Reviews in Therapeutic Drug Carrier Systems, 1991, page 92; Muranishi, Critical Reviews in Therapeutic Drug Carrier Systems, 1990, 7, 1; El-Hariri et al., J. Pharm. Pharmacol., 1992, 44, 651).

A variety of bile salts also function as penetration enhancers to facilitate the uptake and bioavailability of drugs. The physiological roles of bile include the facilitation of dispersion and absorption of lipids and fat-soluble vitamins (Brunton, Chapter 38 In: Goodman & Gilman's The Pharmacological Basis of Therapeutics, 9th Ed., Hardman et al., eds., McGraw-Hill, New York, NY, 1996, pages 934-935). Various natural bile salts, and their synthetic derivatives, act as penetration enhancers. Thus, the term “bile salt” includes any of the naturally occurring components of bile as well as any of their synthetic derivatives. The bile salts of the invention include, for example, cholic acid (or its pharmaceutically acceptable sodium salt, sodium cholate), dehydrocholic acid (sodium dehydrocholate), deoxycholic acid (sodium deoxycholate), glucholic acid (sodium glucholate), glycholic acid (sodium glycocholate), glycodeoxycholic acid (sodium glycodeoxycholate), taurocholic acid (sodium taurocholate), taurodeoxycholic acid (sodium taurodeoxycholate), chenodeoxycholic acid (CDCA, sodium chenodeoxycholate), ursodeoxycholic acid (UDCA), sodium tauro-24,25-dihydro-fusidate (STDHF), sodium glycodihydrofusidate and polyoxyethylene-9-lauryl ether (POE) (Lee et al., Critical Reviews in Therapeutic Drug Carrier Systems, 1991, page 92; Swinyard, Chapter 39 In: Remington's Pharmaceutical Sciences, 18th Ed., Gennaro, ed., Mack Publishing Co., Easton, PA, 1990, pages 782-783; Muranishi, Critical Reviews in Therapeutic Drug Carrier Systems, 1990, 7, 1; Yamamoto et al., J. Pharm. Exp. Ther., 1992, 263, 25; Yamashita et al., J. Pharm. Sci., 1990, 79, 579).

In certain embodiments, penetration enhancers useful in the present invention are mixtures of penetration enhancing compounds. For example, a particularly preferred penetration enhancer is a mixture of UDCA (and/or CDCA) with capric and/or lauric acids or salts thereof e.g. sodium. Such mixtures are useful for enhancing the delivery of biologically active substances across mucosal membranes, in particular intestinal mucosa. Preferred penetration enhancer mixtures comprise about 5-95% of bile acid or salt(s) UDCA and/or CDCA with 5-95% capric and/or lauric acid. Particularly preferred are mixtures of the sodium salts of UDCA, capric acid and lauric acid in a ratio of about 1:2:2 respectively.

In certain embodiments, chelating agents, as used in connection with the present invention, can be defined to be compounds that remove metallic ions from solution by forming complexes therewith, with the result that absorption of conjugated oligomeric compounds through the alimentary and other mucosa is enhanced. With regards to their use as penetration enhancers in the present invention, chelating agents have the added advantage of also serving as DNase inhibitors, as most characterized DNA nucleases require a divalent metal ion for catalysis and are thus inhibited by chelating agents (Jarrett, J Chromatogr., 1993, 618, 315). Chelating agents of the invention include, but are not limited to, disodium ethylenediaminetetraacetate (EDTA), citric acid, salicylates (e.g., sodium salicylate, 5-methoxysalicylate and homovanilate), N-acyl derivatives of collagen, laureth-9 and N-amino acyl derivatives of beta-diketones (enamines)(Lee et al., Critical Reviews in Therapeutic Drug Carrier Systems, 1991, page 92; Muranishi, Critical Reviews in Therapeutic Drug Carrier Systems, 1990, 7, 1; Buur et al., J. Control Rel., 1990, 14, 43).

As used herein, non-chelating non-surfactant penetration enhancers mean compounds that demonstrate insignificant activity as chelating agents or as surfactants but that nonetheless enhance absorption of conjugated oligomeric compounds through the alimentary and other mucosal membranes (Muranishi, Critical Reviews in Therapeutic Drug Carrier Systems, 1990, 7, 1). This class of penetration enhancers includes, but is not limited to, unsaturated cyclic ureas, 1-alkyl- and 1-alkenylazacyclo-alkanone derivatives (Lee et al., Critical Reviews in Therapeutic Drug Carrier Systems, 1991, page 92); and non-steroidal anti-inflammatory agents such as diclofenac sodium, indomethacin and phenylbutazone (Yamashita et al., J Pharm. Pharmacol., 1987, 39, 621).

In certain embodiments, agents that enhance uptake of conjugated oligomeric compounds at the cellular level are added to the compositions of the present invention. For example, cationic lipids, such as lipofectin (Junichi et al, U.S. Pat. No. 5,705,188), cationic glycerol derivatives, and polycationic molecules, such as polylysine (Lollo et al., PCT Application WO 97/30731), can be used.

In certain embodiments, the compositions of the present invention include carrier compounds in the formulation. As used herein, “carrier compound” or “carrier” can refer to a nucleic acid, a conjugated nucleic acid, or analog thereof, which is inert (i.e., does not possess biological activity per se) but is recognized as a nucleic acid by in vivo processes that reduce the bioavailability of an oligomeric compound having biological activity by, for example, degrading the biologically active oligomeric compound or promoting its removal from circulation. The coadministration of a conjugated oligomeric compound and a carrier compound, typically with an excess of the latter substance, can result in a substantial reduction of the amount of nucleic acid recovered in the liver, kidney or other extracirculatory reservoirs, presumably due to competition between the carrier compound and the nucleic acid for a common receptor. For example, the recovery of a partially phosphorothioate oligonucleotide in hepatic tissue can be reduced when it is coadministered with polyinosinic acid, dextran sulfate, polycytidic acid or 4-acetamido-4′isothiocyano-stilbene-2,2′-disulfonic acid (Miyao et al., Antisense Res. Dev., 1995, 5, 115; Takakura et al., Antisense & Nucl. Acid Drug Dev., 1996, 6, 177).

There are three practical mechanisms by which a pharmaceutical formulation can be targeted into the intestine (small intestine or colon) following oral administration: activation by colonic bacterial enzymes or reducing environment created by the microflora, pH-dependent coating and time-dependent coating (coating thickness).

Delayed release coatings, and the properties which influence their dissolution, are well known in the art and are described in, for example, Bauer et al., Coated Pharmaceutical Dosage Forms, Medpharm Scientific Publishers, CRC Press, New York, 1998 and by Watts et al., Drug Devel, Industr. Pharm. 23:893-913, 1997, the entire contents of which are incorporated herein by reference.

In certain embodiments, the compositions of the present invention include other adjunct components conventionally found in pharmaceutical compositions, at their art-established usage levels. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the composition of present invention, such as dyes, flavoring agents, preservatives, antioxidants, opacifiers, thickening agents and stabilizers. However, such materials, when added, should not unduly interfere with the biological activities of the components of the compositions of the present invention.

In certain embodiments, the present invention employs conjugated oligomeric compounds such as conjugated antisense oligonucleotides for use in antisense modulation of the function of DNA or messenger RNA (mRNA) encoding a protein the modulation of which is desired, and ultimately to regulate the amount of such a protein. Hybridization of a conjugated oligomeric compound such as an antisense oligonucleotide with its mRNA target interferes with the normal role of mRNA and causes a modulation of its function in cells. The functions of mRNA to be interfered with include all vital functions such as translocation of the RNA to the site for protein translation, actual translation of protein from the RNA, splicing of the RNA to yield one or more mRNA species, turnover or degradation of the mRNA and possibly even independent catalytic activity which may be engaged in by the RNA. The overall effect of such interference with mRNA function is modulation of the expression of a protein, wherein “modulation” means either an increase (stimulation) or a decrease (inhibition) in the expression of the protein. In the context of the present invention, inhibition is the preferred form of modulation of gene expression.

Capsules used for oral delivery may include formulations that are well known in the art. Further, multicompartment hard capsules with control release properties as described by Digenis et al., U.S. Pat. No. 5,672,359, and water permeable capsules with a multi-stage drug delivery system as described by Amidon et al., U.S. Pat. No. 5,674,530 may also be used to formulate the compositions of the present invention.

Compositions for oral administration include powders or granules, suspensions or solutions in water or non-aqueous media, capsules, sachets, troches, tablets or SECs (soft elastic capsules or “caplets”). Thickeners, flavoring agents, diluents, emulsifiers, dispersing aids, carrier substances or binders may be desirably added to such formulations. A tablet may be made by compression or molding, optionally with one or more accessory ingredients.

Compressed tablets may be prepared by compressing in a suitable machine, the active ingredients in a free flowing form such as a powder or granules, optionally mixed with a binder (PVP or gums such as tragacanth, acacia, carrageenan), lubricant (e.g. stearates such as magnesium stearate), glidant (talc, colloidal silica dioxide), inert diluent, preservative, surface active or dispersing agent. Preferred binders/disintegrants include EMDEX (dextrate), PRECIROL (triglyceride), PEG, and AVICEL (cellulose). Molded tablets may be made by molding in a suitable machine a mixture of the powdered compound moistened with an inert liquid diluent. The tablets may optionally be coated or scored and may be formulated so as to provide slow or controlled release of the active ingredients therein.

Capsules used for oral delivery may include formulations that are well known in the art. Further, multicompartment hard capsules with control release properties as described by Digenis et al., U.S. Pat. No. 5,672,359, and water permeable capsules with a multi-stage drug delivery system as described by Amidon et al., U.S. Pat. No. 5,674,530 may also be used to formulate the compositions of the present invention.

The present invention provides compositions and methods for oral delivery of a drug to an animal. For purposes of the invention, the term “animal” is meant to encompass humans as well as other mammals, as well as reptiles, fish, amphibians, and birds. The compositions of the present invention may be prepared and formulated as emulsions. Emulsions are typically heterogenous systems of one liquid dispersed in another in the form of droplets usually exceeding 0.1 um in diameter. (Idson, in Pharmaceutical Dosage Forms: Disperse Systems, Vol. 1, Lieberman, Rieger and Banker, Eds., Marcel Dekker, Inc., New York, NY, 1988, p. 199; Rosoff, in Pharmaceutical Dosage Forms: Disperse Systems, Vol. 1, Lieberman, Rieger and Banker, Eds., Marcel Dekker, Inc., New York, NY, 1988, p. 245; Block, in Pharmaceutical Dosage Forms: Disperse Systems, Vol. 2, Lieberman, Rieger and Banker, Eds., Marcel Dekker, Inc., New York, NY, 1988, p. 335; Higuchi et al., in “Remington's Pharmaceutical Sciences,” Mack Publishing Co., Easton, PA, 1985, p. 301). Emulsions are often biphasic systems comprising of two immiscible liquid phases intimately mixed and dispersed with each other. In general, emulsions may be either water in oil (w/o) or of the oil in water (o/w) variety. When an aqueous phase is finely divided into and dispersed as minute droplets into a bulk oily phase the resulting composition is called a water in oil (w/o) emulsion. Alternatively, when an oily phase is finely divided into and dispersed as minute droplets into a bulk aqueous phase the resulting composition is called an oil in water (o/w) emulsion.

Emulsions may contain additional components in addition to the dispersed phases and the active drug that may be present as a solution in either the aqueous phase, oily phase or itself as a separate phase. Pharmaceutical excipients such as emulsifiers, stabilizers, dyes, and anti-oxidants may also be present in emulsions as needed. Pharmaceutical emulsions may also be multiple emulsions that are comprised of more than two phases such as, for example, in the case of oil in water in oil (o/w/o) and water in oil in water (w/o/w) emulsions. Such complex formulations often provide certain advantages that simple binary emulsions do not. Multiple emulsions in which individual oil droplets of an o/w emulsion enclose small water droplets constitute a w/o/w emulsion. Likewise a system of oil droplets enclosed in globules of water stabilized in an oily continuous provides an o/w/o emulsion.

In one embodiment of the present invention, the compositions of conjugated oligomeric compounds are formulated as microemulsions. A microemulsion may be defined as a system of water, oil and amphiphile which is a single optically isotropic and thermodynamically stable liquid solution (Rosoff, in Pharmaceutical Dosage Forms: Disperse Systems, Vol. 1, Lieberman, Rieger and Banker, Eds., Marcel Dekker, Inc., New York, NY, 1988, p. 245). Typically microemulsions are systems that are prepared by first dispersing an oil in an aqueous surfactant solution and then adding a sufficient amount of a fourth component, generally an intermediate chain-length alcohol to form a transparent system. Therefore, microemulsions have also been described as thermodynamically stable, isotropically clear dispersions of two immiscible liquids that are stabilized by interfacial films of surface-active molecules (Leung and Shah, in: Controlled Release of Drugs: Polymers and Aggregate Systems, Rosoff, M., Ed., 1989, VCH Publishers, New York, pages 185-215). Microemulsions commonly are prepared via a combination of three to five components that include oil, water, surfactant, cosurfactant and electrolyte. Whether the microemulsion is of the water-in-oil (w/o) or an oil-in-water (o/w) type depends on the properties of the oil and surfactant used and on the structure and geometric packing of the polar heads and hydrocarbon tails of the surfactant molecules (Schott, in “Remington's Pharmaceutical Sciences,” Mack Publishing Co., Easton, PA, 1985, p. 271).

The phenomenological approach utilizing phase diagrams has been extensively studied and has yielded a comprehensive knowledge, to one skilled in the art, of how to formulate microemulsions (Rosoff, in Pharmaceutical Dosage Forms: Disperse Systems, Vol. 1, Lieberman, Rieger and Banker, Eds., Marcel Dekker, Inc., New York, NY, 1988, p. 245; Block, Id., p. 335). Compared to conventional emulsions, microemulsions offer the advantage of solubilizing water-insoluble drugs in a formulation of thermodynamically stable droplets that are formed spontaneously.

Surfactants used in the preparation of microemulsions include, but are not limited to, ionic surfactants, non-ionic surfactants, Brij 96, polyoxyethylene oleyl ethers, polyglycerol fatty acid esters, tetraglycerol monolaurate (ML310), tetraglycerol monooleate (M0310), hexaglycerol monooleate (PO310), hexaglycerol pentaoleate (PO500), decaglycerol monocaprate (MCA750), decaglycerol monooleate (M0750), decaglycerol sequioleate (SO750), decaglycerol decaoleate (DA0750), alone or in combination with cosurfactants. The cosurfactant, usually a short-chain alcohol such as ethanol, 1-propanol, and 1-butanol, serves to increase the interfacial fluidity by penetrating into the surfactant film and consequently creating a disordered film because of the void space generated among surfactant molecules. Microemulsions may, however, be prepared without the use of cosurfactants and alcohol-free self-emulsifying microemulsion systems are known in the art. The aqueous phase may typically be, but is not limited to, water, an aqueous solution of the drug, glycerol, PEG300, PEG400, polyglycerols, propylene glycols, and derivatives of ethylene glycol. The oil phase may include, but is not limited to, materials such as Captex 300, Captex 355, Capmul MCM, fatty acid esters, medium chain (C8-C12) mono, di, and tri-glycerides, polyoxyethylated glyceryl fatty acid esters, fatty alcohols, polyglycolized glycerides, saturated polyglycolized C8-C10 glycerides, vegetable oils and silicone oil.

Microemulsions are particularly of interest from the standpoint of drug solubilization and the enhanced absorption of drugs. Lipid based microemulsions (both o/w and w/o) have been proposed to enhance the oral bioavailability of drugs, including peptides (Constantinides et al., Pharmaceutical Research, 1994, 11, 1385; Ritschel, Meth. Find. Exp. Clin. Pharmacol., 1993, 13, 205). Microemulsions afford advantages of improved drug solubilization, protection of drug from enzymatic hydrolysis, possible enhancement of drug absorption due to surfactant-induced alterations in membrane fluidity and permeability, ease of preparation, ease of oral administration over solid dosage forms, improved clinical potency, and decreased toxicity (Constantinides et al., Pharmaceutical Research, 1994, 11, 1385; Ho et al., J. Pharm. Sci., 1996, 85, 138). Often microemulsions may form spontaneously when their components are brought together at ambient temperature. This may be particularly advantageous when formulating thermolabile drugs, peptides or oligonucleotides. Microemulsions have also been effective in the transdermal delivery of active components in both cosmetic and pharmaceutical applications. It is expected that the microemulsion compositions and formulations of the present invention will facilitate the increased systemic absorption of oligonucleotides and nucleic acids from the gastrointestinal tract, as well as improve the local cellular uptake of oligonucleotides and nucleic acids within the gastrointestinal tract

Microemulsions of the present invention may also contain additional components and additives such as sorbitan monostearate (Grill 3), Labrasol, and penetration enhancers to improve the properties of the formulation and to enhance the absorption of the oligonucleotides and nucleic acids of the present invention. Penetration enhancers used in the microemulsions of the present invention may be classified as belonging to one of five broad categories—surfactants, fatty acids, bile salts, chelating agents, and non-chelating non-surfactants (Lee et al., Critical Reviews in Therapeutic Drug Carrier Systems, 1991, p. 92). Each of these classes has been discussed above.

There are many organized surfactant structures besides microemulsions that have been studied and used for the formulation of drugs. These include monolayers, micelles, bilayers and vesicles. Vesicles, such as liposomes, have attracted great interest because of their specificity and the duration of action they offer from the standpoint of drug delivery. Further advantages are that liposomes obtained from natural phospholipids are biocompatible and biodegradable, liposomes can incorporate a wide range of water and lipid soluble drugs, liposomes can protect encapsulated drugs in their internal compartments from metabolism and degradation (Rosoff, in Pharmaceutical Dosage Forms: Disperse Systems, Vol. 1, Lieberman, Rieger and Banker, Eds., Marcel Dekker, Inc., New York, NY, 1988, p. 245). Important considerations in the preparation of liposome formulations are the lipid surface charge, vesicle size and the aqueous volume of the liposomes. Liposomes can be administered orally and in aerosols and topical applications.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated by reference in their entirety for any purpose.

### Definitions

Unless specific definitions are provided, the nomenclature used in connection with, and the procedures and techniques of, analytical chemistry, synthetic organic chemistry, and medicinal and pharmaceutical chemistry described herein are those well known and commonly used in the art. Standard techniques may be used for chemical synthesis, and chemical analysis. Certain such techniques and procedures may be found for example in “Carbohydrate Modifications in Antisense Research” Edited by Sangvi and Cook, American Chemical Society, Washington D.C., 1994; “Remington's Pharmaceutical Sciences,” Mack Publishing Co., Easton, Pa., 21^{st} edition, 2005; and “Antisense Drug Technology, Principles, Strategies, and Applications” Edited by Stanley T. Crooke, CRC Press, Boca Raton, Florida; and Sambrook et al., “Molecular Cloning, A laboratory Manual,” 2^{nd} Edition, Cold Spring Harbor Laboratory Press, 1989, which are hereby incorporated by reference for any purpose. Where permitted, all patents, applications, published applications and other publications and other data referred to throughout in the disclosure are incorporated by reference herein in their entirety.

Unless otherwise indicated, the following terms have the following meanings:

As used herein, “excipient for oral administration” means any compound or mixture of compounds that is added to a composition as provided herein that is suitable for oral delivery of a conjugated oligomeric compound. In certain embodiments, excipient for oral administration improve bioavailability of a composition as provided herein.

As used herein, “nucleoside” means a compound comprising a nucleobase moiety and a sugar moiety. Nucleosides include, but are not limited to, naturally occurring nucleosides (as found in DNA and RNA) and modified nucleosides. Nucleosides may be linked to a phosphate moiety.

As used herein, “chemical modification” means a chemical difference in a compound when compared to a naturally occurring counterpart. Chemical modifications of oligonucleotides include nucleoside modifications (including sugar moiety modifications and nucleobase modifications) and internucleoside linkage modifications. In reference to an oligonucleotide, chemical modification does not include differences only in nucleobase sequence.

As used herein, “furanosyl” means a structure comprising a 5-membered ring comprising four carbon atoms and one oxygen atom.

As used herein, “naturally occurring sugar moiety” means a ribofuranosyl as found in naturally occurring RNA or a deoxyribofuranosyl as found in naturally occurring DNA.

As used herein, “sugar moiety” means a naturally occurring sugar moiety or a modified sugar moiety of a nucleoside.

As used herein, “modified sugar moiety” means a substituted sugar moiety or a sugar surrogate.

As used herein, “substituted sugar moiety” means a furanosyl that is not a naturally occurring sugar moiety. Substituted sugar moieties include, but are not limited to furanosyls comprising substituents at the 2′-position, the 3′-position, the 5′-position and/or the 4′-position. Certain substituted sugar moieties are bicyclic sugar moieties.

As used herein, “2′-substituted sugar moiety” means a furanosyl comprising a substituent at the 2′-position other than H or OH. Unless otherwise indicated, a 2′-substituted sugar moiety is not a bicyclic sugar moiety (i.e., the 2′-substituent of a 2′-substituted sugar moiety does not form a bridge to another atom of the furanosyl ring.

As used herein, “MOE” means —OCH₂CH₂OCH₃.

As used herein, “2′-F nucleoside” refers to a nucleoside comprising a sugar comprising fluorine at the 2′ position. Unless otherwise indicated, the fluorine in a 2′-F nucleoside is in the ribo position (replacing the OH of a natural ribose).

As used herein the term “sugar surrogate” means a structure that does not comprise a furanosyl and that is capable of replacing the naturally occurring sugar moiety of a nucleoside, such that the resulting nucleoside sub-units are capable of linking together and/or linking to other nucleosides to form an oligomeric compound which is capable of hybridizing to a complementary oligomeric compound. Such structures include rings comprising a different number of atoms than furanosyl (e.g., 4, 6, or 7-membered rings); replacement of the oxygen of a furanosyl with a non-oxygen atom (e.g., carbon, sulfur, or nitrogen); or both a change in the number of atoms and a replacement of the oxygen. Such structures may also comprise substitutions corresponding to those described for substituted sugar moieties (e.g., 6-membered carbocyclic bicyclic sugar surrogates optionally comprising additional substituents). Sugar surrogates also include more complex sugar replacements (e.g., the non-ring systems of peptide nucleic acid). Sugar surrogates include without limitation morpholinos, cyclohexenyls and cyclohexitols.

As used herein, “bicyclic sugar moiety” means a modified sugar moiety comprising a 4 to 7 membered ring (including but not limited to a furanosyl) comprising a bridge connecting two atoms of the 4 to 7 membered ring to form a second ring, resulting in a bicyclic structure. In certain embodiments, the 4 to 7 membered ring is a sugar ring. In certain embodiments the 4 to 7 membered ring is a furanosyl. In certain such embodiments, the bridge connects the 2′-carbon and the 4′-carbon of the furanosyl.

As used herein, “nucleotide” means a nucleoside further comprising a phosphate linking group. As used herein, “linked nucleosides” may or may not be linked by phosphate linkages and thus includes, but is not limited to “linked nucleotides.” As used herein, “linked nucleosides” are nucleosides that are connected in a continuous sequence (i.e. no additional nucleosides are present between those that are linked).

As used herein, “nucleobase” means a group of atoms that can be linked to a sugar moiety to create a nucleoside that is capable of incorporation into an oligonucleotide, and wherein the group of atoms is capable of bonding with a complementary naturally occurring nucleobase of another oligonucleotide or nucleic acid. Nucleobases may be naturally occurring or may be modified.

As used herein the terms, “unmodified nucleobase” or “naturally occurring nucleobase” means the naturally occurring heterocyclic nucleobases of RNA or DNA: the purine bases adenine (A) and guanine (G), and the pyrimidine bases thymine (T), cytosine (C) (including 5-methyl C), and uracil (U).

As used herein, “modified nucleobase” means any nucleobase that is not a naturally occurring nucleobase.

As used herein, “modified nucleoside” means a nucleoside comprising at least one chemical modification compared to naturally occurring RNA or DNA nucleosides. Modified nucleosides comprise a modified sugar moiety and/or a modified nucleobase.

As used herein, “bicyclic nucleoside” or “BNA” means a nucleoside comprising a bicyclic sugar moiety.

As used herein, “constrained ethyl nucleoside” or “cEt” means a nucleoside comprising a bicyclic sugar moiety comprising a 4′-CH(CH₃)—O-2′bridge.

As used herein, “locked nucleic acid nucleoside” or “LNA” means a nucleoside comprising a bicyclic sugar moiety comprising a 4′-CH₂—O-2′bridge.

As used herein, “2′-substituted nucleoside” means a nucleoside comprising a substituent at the 2′-position other than H or OH. Unless otherwise indicated, a 2′-substituted nucleoside is not a bicyclic nucleoside.

As used herein, “deoxynucleoside” means a nucleoside comprising 2′-H furanosyl sugar moiety, as found in naturally occurring deoxyribonucleosides (DNA). In certain embodiments, a 2′-deoxynucleoside may comprise a modified nucleobase or may comprise an RNA nucleobase (e.g., uracil).

As used herein, “oligonucleotide” means a compound comprising a plurality of linked nucleosides.

In certain embodiments, an oligonucleotide comprises one or more unmodified ribonucleosides (RNA) and/or unmodified deoxyribonucleosides (DNA) and/or one or more modified nucleosides.

As used herein “oligonucleoside” means an oligonucleotide in which none of the internucleoside linkages contains a phosphorus atom. As used herein, oligonucleotides include oligonucleosides.

As used herein, “modified oligonucleotide” means an oligonucleotide comprising at least one modified nucleoside and/or at least one modified internucleoside linkage.

As used herein, “linkage” or “linking group” means a group of atoms that link together two or more other groups of atoms. In certain embodiments, a linking group links together a conjugate and a oligomeric compound.

As used herein “internucleoside linkage” means a covalent linkage between adjacent nucleosides in an oligonucleotide.

As used herein “naturally occurring internucleoside linkage” means a 3′ to 5′ phosphodiester linkage.

As used herein, “modified internucleoside linkage” means any internucleoside linkage other than a naturally occurring internucleoside linkage.

As used herein, “terminal internucleoside linkage” means the linkage between the last two nucleosides of an oligonucleotide or defined region thereof.

As used herein, “phosphorus linking group” means a linking group comprising a phosphorus atom. Phosphorus linking groups include without limitation groups having the formula:

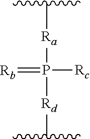

wherein:
Rₐ and R_{d} are each, independently, O, S, CH₂, NH, or NJ₁ wherein J₁ is C₁-C₆ alkyl or substituted C₁-C₆ alkyl;
R_{b} is O or S;
R_{c} is OH, SH, C₁-C₆ alkyl, substituted C₁-C₆ alkyl, C₁-C₆ alkoxy, substituted C₁-C₆ alkoxy, amino or substituted amino; and
J₁ is R_{b} is O or S.

Phosphorus linking groups include without limitation, phosphodiester, phosphorothioate, phosphorodithioate, phosphonate, phosphoramidate, phosphorothioamidate, thionoalkylphosphonate, phosphotriesters, thionoalkylphosphotriester and boranophosphate.

As used herein, “internucleoside phosphorus linking group” means a phosphorus linking group that directly links two nucleosides.

As used herein, “non-internucleoside phosphorus linking group” means a phosphorus linking group that does not directly link two nucleosides. In certain embodiments, a non-internucleoside phosphorus linking group links a nucleoside to a group other than a nucleoside. In certain embodiments, a non-internucleoside phosphorus linking group links two groups, neither of which is a nucleoside.

As used herein, “neutral linking group” means a linking group that is not charged. Neutral linking groups include without limitation phosphotriesters, methylphosphonates, MMI (—CH₂—N(CH₃)—O—), amide-3 (—CH₂—C(═O)—N(H)—), amide-4 (—CH₂—N(H)—C(═O)—), formacetal (—O—CH₂—O—), and thioformacetal (—S—CH₂—O—). Further neutral linking groups include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook Eds. ACS Symposium Series 580; Chapters 3 and 4, (pp. 40-65)). Further neutral linking groups include nonionic linkages comprising mixed N, O, S and CH₂ component parts.

As used herein, “internucleoside neutral linking group” means a neutral linking group that directly links two nucleosides.

As used herein, “non-internucleoside neutral linking group” means a neutral linking group that does not directly link two nucleosides. In certain embodiments, a non-internucleoside neutral linking group links a nucleoside to a group other than a nucleoside. In certain embodiments, a non-internucleoside neutral linking group links two groups, neither of which is a nucleoside.

As used herein, “oligomeric compound” means a polymeric structure comprising two or more sub-structures. In certain embodiments, an oligomeric compound comprises an oligonucleotide. In certain embodiments, an oligomeric compound comprises one or more conjugate groups and/or terminal groups. In certain embodiments, an oligomeric compound consists of an oligonucleotide. Oligomeric compounds also include naturally occurring nucleic acids. In certain embodiments, an oligomeric compound comprises a backbone of one or more linked monomeric subunits where each linked monomeric subunit is directly or indirectly attached to a heterocyclic base moiety. In certain embodiments, oligomeric compounds may also include monomeric subunits that are not linked to a heterocyclic base moiety, thereby providing abasic sites. In certain embodiments, the linkages joining the monomeric subunits, the sugar moieties or surrogates and the heterocyclic base moieties can be independently modified. In certain embodiments, the linkage-sugar unit, which may or may not include a heterocyclic base, may be substituted with a mimetic such as the monomers in peptide nucleic acids.

As used herein, “oligomer” means any compound that comprises at least two linked subunits. In certain embodiments, an oligomeric compound comprises an oligonucleotide. In certain embodiments, an oligomeric compound comprises a modified oligonucleotide. In certain embodiments, an oligomeric compound consists of a modified oligonucleotide.

As used herein, “connecting group” means a bond or a group of atoms that link together two or more other groups of atoms. In certain embodiments, a connecting group links a ligand to a modified oligonucleotide. In certain embodiments, a connecting group comprises all or part of a linking group, a branching group, and/or a tether.

As used herein, “terminal group” means one or more atom attached to either, or both, the 3′ end or the 5′ end of an oligonucleotide. In certain embodiments a terminal group is a conjugate group. In certain embodiments, a terminal group comprises one or more terminal group nucleosides.

As used herein, “conjugate” or “conjugate group” means an atom or group of atoms bound to an oligonucleotide or oligomeric compound. In general, conjugate groups modify one or more properties of the compound to which they are attached, including, but not limited to pharmacodynamic, pharmacokinetic, binding, absorption, cellular distribution, cellular uptake, charge and/or clearance properties.

As used herein, “conjugate linker” or “linker” in the context of a conjugate group means a portion of a conjugate group comprising any atom or group of atoms and which covalently link (1) an oligonucleotide to another portion of the conjugate group or (2) two or more portions of the conjugate group.

Conjugate groups are shown herein as radicals, providing a bond for forming covalent attachment to an oligomeric compound such as an antisense oligonucleotide. In certain embodiments, the point of attachment on the oligomeric compound is the 3′-oxygen atom of the 3′-hydroxyl group of the 3′ terminal nucleoside of the oligomeric compound. In certain embodiments the point of attachment on the oligomeric compound is the 5′-oxygen atom of the 5′-hydroxyl group of the 5′ terminal nucleoside of the oligomeric compound. In certain embodiments, the bond for forming attachment to the oligomeric compound is a cleavable bond. In certain such embodiments, such cleavable bond constitutes all or part of a cleavable moiety.

In certain embodiments, conjugate groups comprise a cleavable moiety (e.g., a cleavable bond or cleavable nucleoside) and a carbohydrate cluster portion, such as a GalNAc cluster portion. Such carbohydrate cluster portion comprises: a targeting moiety and, optionally, a conjugate linker. In certain embodiments, the carbohydrate cluster portion is identified by the number and identity of the ligand. For example, in certain embodiments, the carbohydrate cluster portion comprises 3 GalNAc groups and is designated “GalNAc₃”. In certain embodiments, the carbohydrate cluster portion comprises 4 GalNAc groups and is designated “GalNAc₄”. Specific carbohydrate cluster portions (having specific tether, branching and conjugate linker groups) are described herein and designated by Roman numeral followed by subscript “a”. Accordingly “GalNac3-1ₐ” refers to a specific carbohydrate cluster portion of a conjugate group having 3 GalNac groups and specifically identified tether, branching and linking groups. Such carbohydrate cluster fragment is attached to an oligomeric compound via a cleavable moiety, such as a cleavable bond or cleavable nucleoside.

As used herein, “cleavable moiety” means a bond or group that is capable of being cleaved under physiological conditions. In certain embodiments, a cleavable moiety is cleaved inside a cell or sub-cellular compartments, such as an endosome or lysosome. In certain embodiments, a cleavable moiety is cleaved by endogenous enzymes, such as nucleases. In certain embodiments, a cleavable moiety comprises a group of atoms having one, two, three, four, or more than four cleavable bonds. In certain embodiments, a cleavable moiety is a phosphodiester linkage.

As used herein, “cleavable bond” means any chemical bond capable of being broken. In certain embodiments, a cleavable bond is selected from among: an amide, a polyamide, an ester, an ether, one or both esters of a phosphodiester, a phosphate ester, a carbamate, a di-sulfide, or a peptide.

As used herein, “carbohydrate cluster” means a compound having one or more carbohydrate residues attached to a scaffold or linker group. (see, e.g., Maier et al., “Synthesis of Antisense Oligonucleotides Conjugated to a Multivalent Carbohydrate Cluster for Cellular Targeting,” Bioconjugate Chemistry, 2003, (14): 18-29, which is incorporated herein by reference in its entirety, or Rensen et al., “Design and Synthesis of Novel N-Acetylgalactosamine-Terminated Glycolipids for Targeting of Lipoproteins to the Hepatic Asiaglycoprotein Receptor,” J. Med. Chem. 2004, (47): 5798-5808, for examples of carbohydrate conjugate clusters).

As used herein, “modified carbohydrate” means any carbohydrate having one or more chemical modifications relative to naturally occurring carbohydrates.

As used herein, “carbohydrate derivative” means any compound which may be synthesized using a carbohydrate as a starting material or intermediate.

As used herein, “carbohydrate” means a naturally occurring carbohydrate, a modified carbohydrate, or a carbohydrate derivative.

As used herein “protecting group” means any compound or protecting group known to those having skill in the art. Non-limiting examples of protecting groups may be found in “Protective Groups in Organic Chemistry”, T. W. Greene, P. G. M. Wuts, ISBN 0-471-62301-6, John Wiley & Sons, Inc, New York, which is incorporated herein by reference in its entirety.

As used herein, “single-stranded” means an oligomeric compound that is not hybridized to its complement and which lacks sufficient self-complementarity to form a stable self-duplex.

As used herein, “double stranded” means a pair of oligomeric compounds that are hybridized to one another or a single self-complementary oligomeric compound that forms a hairpin structure. In certain embodiments, a double-stranded oligomeric compound comprises a first and a second oligomeric compound.

As used herein, “antisense compound” means a compound comprising or consisting of an oligonucleotide at least a portion of which is complementary to a target nucleic acid to which it is capable of hybridizing, resulting in at least one antisense activity.

As used herein, “antisense activity” means any detectable and/or measurable change attributable to the hybridization of an antisense compound to its target nucleic acid. In certain embodiments, antisense activity includes modulation of the amount or activity of a target nucleic acid transcript (e.g. mRNA). In certain embodiments, antisense activity includes modulation of the splicing of pre-mRNA.

As used herein, “RNase H based antisense compound” means an antisense compound wherein at least some of the antisense activity of the antisense compound is attributable to hybridization of the antisense compound to a target nucleic acid and subsequent cleavage of the target nucleic acid by RNase H.

As used herein, “RISC based antisense compound” means an antisense compound wherein at least some of the antisense activity of the antisense compound is attributable to the RNA Induced Silencing Complex (RISC).

As used herein, “detecting” or “measuring” means that a test or assay for detecting or measuring is performed. Such detection and/or measuring may result in a value of zero. Thus, if a test for detection or measuring results in a finding of no activity (activity of zero), the step of detecting or measuring the activity has nevertheless been performed.

As used herein, “detectable and/or measureable activity” means a statistically significant activity that is not zero.

As used herein, “essentially unchanged” means little or no change in a particular parameter, particularly relative to another parameter which changes much more. In certain embodiments, a parameter is essentially unchanged when it changes less than 5%. In certain embodiments, a parameter is essentially unchanged if it changes less than two-fold while another parameter changes at least ten-fold. For example, in certain embodiments, an antisense activity is a change in the amount of a target nucleic acid. In certain such embodiments, the amount of a non-target nucleic acid is essentially unchanged if it changes much less than the target nucleic acid does, but the change need not be zero.

As used herein, “expression” means the process by which a gene ultimately results in a protein. Expression includes, but is not limited to, transcription, post-transcriptional modification (e.g., splicing, polyadenlyation, addition of 5′-cap), and translation.

As used herein, “target nucleic acid” means a nucleic acid molecule to which an antisense compound is intended to hybridize to result in a desired antisense activity. Antisense oligonucleotides have sufficient complementarity to their target nucleic acids to allow hybridization under physiological conditions.

As used herein, “nucleobase complementarity” or “complementarity” when in reference to nucleobases means a nucleobase that is capable of base pairing with another nucleobase. For example, in DNA, adenine (A) is complementary to thymine (T). For example, in RNA, adenine (A) is complementary to uracil (U). In certain embodiments, complementary nucleobase means a nucleobase of an antisense compound that is capable of base pairing with a nucleobase of its target nucleic acid. For example, if a nucleobase at a certain position of an antisense compound is capable of hydrogen bonding with a nucleobase at a certain position of a target nucleic acid, then the position of hydrogen bonding between the oligonucleotide and the target nucleic acid is considered to be complementary at that nucleobase pair. Nucleobases comprising certain modifications may maintain the ability to pair with a counterpart nucleobase and thus, are still capable of nucleobase complementarity.

As used herein, “non-complementary” in reference to nucleobases means a pair of nucleobases that do not form hydrogen bonds with one another.

As used herein, “complementary” in reference to oligomeric compounds (e.g., linked nucleosides, oligonucleotides, or nucleic acids) means the capacity of such oligomeric compounds or regions thereof to hybridize to another oligomeric compound or region thereof through nucleobase complementarity. Complementary oligomeric compounds need not have nucleobase complementarity at each nucleoside. Rather, some mismatches are tolerated. In certain embodiments, complementary oligomeric compounds or regions are complementary at 70% of the nucleobases (70% complementary). In certain embodiments, complementary oligomeric compounds or regions are 80% complementary. In certain embodiments, complementary oligomeric compounds or regions are 90% complementary. In certain embodiments, complementary oligomeric compounds or regions are 95% complementary. In certain embodiments, complementary oligomeric compounds or regions are 100% complementary.

As used herein, “mismatch” means a nucleobase of a first oligomeric compound that is not capable of pairing with a nucleobase at a corresponding position of a second oligomeric compound, when the first and second oligomeric compound are aligned. Either or both of the first and second oligomeric compounds may be oligonucleotides.

As used herein, “hybridization” means the pairing of complementary oligomeric compounds (e.g., an antisense compound and its target nucleic acid). While not limited to a particular mechanism, the most common mechanism of pairing involves hydrogen bonding, which may be Watson-Crick, Hoogsteen or reversed Hoogsteen hydrogen bonding, between complementary nucleobases.

As used herein, “specifically hybridizes” means the ability of an oligomeric compound to hybridize to one nucleic acid site with greater affinity than it hybridizes to another nucleic acid site.

As used herein, “fully complementary” in reference to an oligonucleotide or portion thereof means that each nucleobase of the oligonucleotide or portion thereof is capable of pairing with a nucleobase of a complementary nucleic acid or contiguous portion thereof. Thus, a fully complementary region comprises no mismatches or unhybridized nucleobases in either strand.

As used herein, “percent complementarity” means the percentage of nucleobases of an oligomeric compound that are complementary to an equal-length portion of a target nucleic acid. Percent complementarity is calculated by dividing the number of nucleobases of the oligomeric compound that are complementary to nucleobases at corresponding positions in the target nucleic acid by the total length of the oligomeric compound.

As used herein, “percent identity” means the number of nucleobases in a first nucleic acid that are the same type (independent of chemical modification) as nucleobases at corresponding positions in a second nucleic acid, divided by the total number of nucleobases in the first nucleic acid.

As used herein, “modulation” means a change of amount or quality of a molecule, function, or activity when compared to the amount or quality of a molecule, function, or activity prior to modulation. For example, modulation includes the change, either an increase (stimulation or induction) or a decrease (inhibition or reduction) in gene expression. As a further example, modulation of expression can include a change in splice site selection of pre-mRNA processing, resulting in a change in the absolute or relative amount of a particular splice-variant compared to the amount in the absence of modulation.

As used herein, “chemical motif” means a pattern of chemical modifications in an oligonucleotide or a region thereof. Motifs may be defined by modifications at certain nucleosides and/or at certain linking groups of an oligonucleotide.

As used herein, “nucleoside motif” means a pattern of nucleoside modifications in an oligonucleotide or a region thereof. The linkages of such an oligonucleotide may be modified or unmodified. Unless otherwise indicated, motifs herein describing only nucleosides are intended to be nucleoside motifs. Thus, in such instances, the linkages are not limited.

As used herein, “sugar motif” means a pattern of sugar modifications in an oligonucleotide or a region thereof.

As used herein, “linkage motif” means a pattern of linkage modifications in an oligonucleotide or region thereof. The nucleosides of such an oligonucleotide may be modified or unmodified. Unless otherwise indicated, motifs herein describing only linkages are intended to be linkage motifs. Thus, in such instances, the nucleosides are not limited.

As used herein, “nucleobase modification motif” means a pattern of modifications to nucleobases along an oligonucleotide. Unless otherwise indicated, a nucleobase modification motif is independent of the nucleobase sequence.

As used herein, “sequence motif” means a pattern of nucleobases arranged along an oligonucleotide or portion thereof. Unless otherwise indicated, a sequence motif is independent of chemical modifications and thus may have any combination of chemical modifications, including no chemical modifications.

As used herein, “type of modification” in reference to a nucleoside or a nucleoside of a “type” means the chemical modification of a nucleoside and includes modified and unmodified nucleosides. Accordingly, unless otherwise indicated, a “nucleoside having a modification of a first type” may be an unmodified nucleoside.

As used herein, “differently modified” mean chemical modifications or chemical substituents that are different from one another, including absence of modifications. Thus, for example, a MOE nucleoside and an unmodified DNA nucleoside are “differently modified,” even though the DNA nucleoside is unmodified. Likewise, DNA and RNA are “differently modified,” even though both are naturally-occurring unmodified nucleosides. Nucleosides that are the same but for comprising different nucleobases are not differently modified. For example, a nucleoside comprising a 2′-OMe modified sugar and an unmodified adenine nucleobase and a nucleoside comprising a 2′-OMe modified sugar and an unmodified thymine nucleobase are not differently modified.

As used herein, “the same type of modifications” refers to modifications that are the same as one another, including absence of modifications. Thus, for example, two unmodified DNA nucleosides have “the same type of modification,” even though the DNA nucleoside is unmodified. Such nucleosides having the same type modification may comprise different nucleobases.

As used herein, “separate regions” means portions of an oligonucleotide wherein the chemical modifications or the motif of chemical modifications of any neighboring portions include at least one difference to allow the separate regions to be distinguished from one another.

As used herein, “pharmaceutically acceptable carrier or diluent” means any substance suitable for use in administering to an animal. In certain embodiments, a pharmaceutically acceptable carrier or diluent is sterile saline. In certain embodiments, such sterile saline is pharmaceutical grade saline.

As used herein the term “metabolic disorder” means a disease or condition principally characterized by dysregulation of metabolism—the complex set of chemical reactions associated with breakdown of food to produce energy.

As used herein, the term “cardiovascular disorder” means a disease or condition principally characterized by impaired function of the heart or blood vessels.

As used herein the term “mono or polycyclic ring system” is meant to include all ring systems selected from single or polycyclic radical ring systems wherein the rings are fused or linked and is meant to be inclusive of single and mixed ring systems individually selected from aliphatic, alicyclic, aryl, heteroaryl, aralkyl, arylalkyl, heterocyclic, heteroaryl, heteroaromatic and heteroarylalkyl. Such mono and poly cyclic structures can contain rings that each have the same level of saturation or each, independently, have varying degrees of saturation including fully saturated, partially saturated or fully unsaturated. Each ring can comprise ring atoms selected from C, N, O and S to give rise to heterocyclic rings as well as rings comprising only C ring atoms which can be present in a mixed motif such as for example benzimidazole wherein one ring has only carbon ring atoms and the fused ring has two nitrogen atoms. The mono or polycyclic ring system can be further substituted with substituent groups such as for example phthalimide which has two ═O groups attached to one of the rings. Mono or polycyclic ring systems can be attached to parent molecules using various strategies such as directly through a ring atom, fused through multiple ring atoms, through a substituent group or through a bifunctional linking moiety.

As used herein, “prodrug” means an inactive or less active form of a compound which, when administered to a subject, is metabolized to form the active, or more active, compound (e.g., drug).

As used herein, “substituent” and “substituent group,” means an atom or group that replaces the atom or group of a named parent compound. For example a substituent of a modified nucleoside is any atom or group that differs from the atom or group found in a naturally occurring nucleoside (e.g., a modified 2′-substituent is any atom or group at the 2′-position of a nucleoside other than H or OH). Substituent groups can be protected or unprotected. In certain embodiments, compounds of the present disclosure have substituents at one or at more than one position of the parent compound. Substituents may also be further substituted with other substituent groups and may be attached directly or via a linking group such as an alkyl or hydrocarbyl group to a parent compound.

Likewise, as used herein, “substituent” in reference to a chemical functional group means an atom or group of atoms that differs from the atom or a group of atoms normally present in the named functional group. In certain embodiments, a substituent replaces a hydrogen atom of the functional group (e.g., in certain embodiments, the substituent of a substituted methyl group is an atom or group other than hydrogen which replaces one of the hydrogen atoms of an unsubstituted methyl group). Unless otherwise indicated, groups amenable for use as substituents include without limitation, halogen, hydroxyl, alkyl, alkenyl, alkynyl, acyl (—C(O)Rₐₐ), carboxyl (—C(O)O—Rₐₐ), aliphatic groups, alicyclic groups, alkoxy, substituted oxy (—O—Rₐₐ), aryl, aralkyl, heterocyclic radical, heteroaryl, heteroarylalkyl, amino (—N(R_{bb})(R_{cc})), imino(=NR_{bb}), amido (—C(O)N(R_{bb})(R_{cc}) or —N(R_{bb})C(O)Rₐₐ), azido (—N₃), nitro (—NO₂), cyano (—CN), carbamido (—OC(O)N(R_{bb})(R_{cc}) or —N(R_{bb})C(O)ORₐₐ), ureido (—N(R_{bb})C(O)N(R_{bb})(R_{cc})), thioureido (—N(R_{bb})C(S)N(R_{bb})—(R_{cc})), guanidinyl (—N(R_{bb})C(═NR_{bb})N(R_{bb})(R_{cc})), amidinyl (—C(═NR_{bb})N(R_{bb})(R_{cc}) or —N(R_{bb})C(═NR_{bb})(Rₐₐ)), thiol (—SR_{bb}), sulfinyl (—S(O)R_{bb}), sulfonyl (—S(O)₂R_{bb}) and sulfonamidyl (—S(O)₂N(R_{bb})(R_{cc}) or —N(R_{bb})S—(O)₂R_{bb}). Wherein each Rₐₐ, R_{bb} and R_{cc} is, independently, H, an optionally linked chemical functional group or a further substituent group with a preferred list including without limitation, alkyl, alkenyl, alkynyl, aliphatic, alkoxy, acyl, aryl, aralkyl, heteroaryl, alicyclic, heterocyclic and heteroarylalkyl. Selected substituents within the compounds described herein are present to a recursive degree.

As used herein, “alkyl,” as used herein, means a saturated straight or branched hydrocarbon radical containing up to twenty four carbon atoms. Examples of alkyl groups include without limitation, methyl, ethyl, propyl, butyl, isopropyl, n-hexyl, octyl, decyl, dodecyl and the like. Alkyl groups typically include from 1 to about 24 carbon atoms, more typically from 1 to about 12 carbon atoms (C₁-C₁₂ alkyl) with from 1 to about 6 carbon atoms being more preferred.

As used herein, “alkenyl,” means a straight or branched hydrocarbon chain radical containing up to twenty four carbon atoms and having at least one carbon-carbon double bond. Examples of alkenyl groups include without limitation, ethenyl, propenyl, butenyl, 1-methyl-2-buten-1-yl, dienes such as 1,3-butadiene and the like. Alkenyl groups typically include from 2 to about 24 carbon atoms, more typically from 2 to about 12 carbon atoms with from 2 to about 6 carbon atoms being more preferred. Alkenyl groups as used herein may optionally include one or more further substituent groups.

As used herein, “alkynyl,” means a straight or branched hydrocarbon radical containing up to twenty four carbon atoms and having at least one carbon-carbon triple bond. Examples of alkynyl groups include, without limitation, ethynyl, 1-propynyl, 1-butynyl, and the like. Alkynyl groups typically include from 2 to about 24 carbon atoms, more typically from 2 to about 12 carbon atoms with from 2 to about 6 carbon atoms being more preferred. Alkynyl groups as used herein may optionally include one or more further substituent groups.

As used herein, “acyl,” means a radical formed by removal of a hydroxyl group from an organic acid and has the general Formula —C(O)—X where X is typically aliphatic, alicyclic or aromatic. Examples include aliphatic carbonyls, aromatic carbonyls, aliphatic sulfonyls, aromatic sulfinyls, aliphatic sulfinyls, aromatic phosphates, aliphatic phosphates and the like. Acyl groups as used herein may optionally include further substituent groups.

As used herein, “alicyclic” means a cyclic ring system wherein the ring is aliphatic. The ring system can comprise one or more rings wherein at least one ring is aliphatic. Preferred alicyclics include rings having from about 5 to about 9 carbon atoms in the ring. Alicyclic as used herein may optionally include further substituent groups.

As used herein, “aliphatic” means a straight or branched hydrocarbon radical containing up to twenty four carbon atoms wherein the saturation between any two carbon atoms is a single, double or triple bond. An aliphatic group preferably contains from 1 to about 24 carbon atoms, more typically from 1 to about 12 carbon atoms with from 1 to about 6 carbon atoms being more preferred. The straight or branched chain of an aliphatic group may be interrupted with one or more heteroatoms that include nitrogen, oxygen, sulfur and phosphorus. Such aliphatic groups interrupted by heteroatoms include without limitation, polyalkoxys, such as polyalkylene glycols, polyamines, and polyimines. Aliphatic groups as used herein may optionally include further substituent groups.

As used herein, “alkoxy” means a radical formed between an alkyl group and an oxygen atom wherein the oxygen atom is used to attach the alkoxy group to a parent molecule. Examples of alkoxy groups include without limitation, methoxy, ethoxy, propoxy, isopropoxy, n-butoxy, sec-butoxy, tert-butoxy, n-pentoxy, neopentoxy, n-hexoxy and the like. Alkoxy groups as used herein may optionally include further substituent groups.

As used herein, “aminoalkyl” means an amino substituted C₁-C₁₂ alkyl radical. The alkyl portion of the radical forms a covalent bond with a parent molecule. The amino group can be located at any position and the aminoalkyl group can be substituted with a further substituent group at the alkyl and/or amino portions.

As used herein, “aralkyl” and “arylalkyl” mean an aromatic group that is covalently linked to a C₁-C₁₂ alkyl radical. The alkyl radical portion of the resulting aralkyl (or arylalkyl) group forms a covalent bond with a parent molecule. Examples include without limitation, benzyl, phenethyl and the like. Aralkyl groups as used herein may optionally include further substituent groups attached to the alkyl, the aryl or both groups that form the radical group.

As used herein, “aryl” and “aromatic” mean a mono- or polycyclic carbocyclic ring system radicals having one or more aromatic rings. Examples of aryl groups include without limitation, phenyl, naphthyl, tetrahydronaphthyl, indanyl, idenyl and the like. Preferred aryl ring systems have from about 5 to about 20 carbon atoms in one or more rings. Aryl groups as used herein may optionally include further substituent groups.

As used herein, “halo” and “halogen,” mean an atom selected from fluorine, chlorine, bromine and iodine.

As used herein, “heteroaryl,” and “heteroaromatic,” mean a radical comprising a mono- or poly-cyclic aromatic ring, ring system or fused ring system wherein at least one of the rings is aromatic and includes one or more heteroatoms. Heteroaryl is also meant to include fused ring systems including systems where one or more of the fused rings contain no heteroatoms. Heteroaryl groups typically include one ring atom selected from sulfur, nitrogen or oxygen. Examples of heteroaryl groups include without limitation, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzooxazolyl, quinoxalinyl and the like. Heteroaryl radicals can be attached to a parent molecule directly or through a linking moiety such as an aliphatic group or hetero atom. Heteroaryl groups as used herein may optionally include further substituent groups.

As used herein, “conjugate compound” means any atoms, group of atoms, or group of linked atoms suitable for use as a conjugate group. In certain embodiments, conjugate compounds may possess or impart one or more properties, including, but not limited to pharmacodynamic, pharmacokinetic, binding, absorption, cellular distribution, cellular uptake, charge and/or clearance properties.

As used herein, unless otherwise indicated or modified, the term “double-stranded” refers to two separate oligomeric compounds that are hybridized to one another. Such double stranded compounds may have one or more or non-hybridizing nucleosides at one or both ends of one or both strands (overhangs) and/or one or more internal non-hybridizing nucleosides (mismatches) provided there is sufficient complementarity to maintain hybridization under physiologically relevant conditions.

### Certain Compounds

In certain embodiments, the invention provides conjugated antisense compounds comprising antisense oligonucleoitdes and a conjugate.

### Certain Antisense Oligonucleotides

In certain embodiments, the invention provides antisense oligonucleotides. Such antisense oligonucleotides comprise linked nucleosides, each nucleoside comprising a sugar moiety and a nucleobase. The structure of such antisense oligonucleotides may be considered in terms of chemical features (e.g., modifications and patterns of modifications) and nucleobase sequence (e.g., sequence of antisense oligonucleotide, identity and sequence of target nucleic acid).

### Certain Chemistry Features

In certain embodiments, antisense oligonucleotide comprise one or more modification. In certain such embodiments, antisense oligonucleotides comprise one or more modified nucleosides and/or modified internucleoside linkages. In certain embodiments, modified nucleosides comprise a modified sugar moiety and/or modified nucleobase.

### Certain Sugar Moieties

In certain embodiments, compounds of the disclosure comprise one or more modified nucleosides comprising a modified sugar moiety. Such compounds comprising one or more sugar-modified nucleosides may have desirable properties, such as enhanced nuclease stability or increased binding affinity with a target nucleic acid relative to an oligonucleotide comprising only nucleosides comprising naturally occurring sugar moieties. In certain embodiments, modified sugar moieties are substituted sugar moieties. In certain embodiments, modified sugar moieties are sugar surrogates. Such sugar surrogates may comprise one or more substitutions corresponding to those of substituted sugar moieties.

In certain embodiments, modified sugar moieties are substituted sugar moieties comprising one or more non-bridging sugar substituent, including but not limited to substituents at the 2′ and/or 5′ positions. Examples of sugar substituents suitable for the 2′-position, include, but are not limited to: 2′-F, 2′-OCH₃ (“OMe” or “O-methyl”), and 2′-O(CH₂)₂OCH₃ (“MOE”). In certain embodiments, sugar substituents at the 2′ position is selected from allyl, amino, azido, thio, O-allyl, O—C₁-C₁₀ alkyl, O—C₁-C₁₀ substituted alkyl; OCF₃, O(CH₂)₂SCH₃, O(CH₂)₂—O—N(Rₘ)(Rₙ), and O—CH₂—C(═O)—N(Rₘ)(Rₙ), where each Rₘ and Rₙ is, independently, H or substituted or unsubstituted C₁-C₁₀ alkyl. Examples of sugar substituents at the 5′-position, include, but are not limited to: 5′-methyl (R or S); 5′-vinyl, and 5′-methoxy. In certain embodiments, substituted sugars comprise more than one non-bridging sugar substituent, for example, 2′-F-5′-methyl sugar moieties (see, e.g., PCT International Application WO 2008/101157, for additional 5′, 2′-bis substituted sugar moieties and nucleosides).

Nucleosides comprising 2′-substituted sugar moieties are referred to as 2′-substituted nucleosides. In certain embodiments, a 2′-substituted nucleoside comprises a 2′-substituent group selected from halo, allyl, amino, azido, SH, CN, OCN, CF₃, OCF₃, O, S, or N(Rₘ)-alkyl; O, S, or N(Rₘ)-alkenyl; O, S or N(Rₘ)-alkynyl; O-alkylenyl-O-alkyl, alkynyl, alkaryl, aralkyl, O-alkaryl, O-aralkyl, O(CH₂)₂SCH₃, O—(CH₂)₂—O—N(Rₘ)(Rₙ) or O—CH₂—C(═O)—N(Rₘ)(Rₙ), where each Rₘ and Rₙ is, independently, H, an amino protecting group or substituted or unsubstituted C₁-C₁₀ alkyl. These 2′-substituent groups can be further substituted with one or more substituent groups independently selected from hydroxyl, amino, alkoxy, carboxy, benzyl, phenyl, nitro (NO₂), thiol, thioalkoxy (S-alkyl), halogen, alkyl, aryl, alkenyl and alkynyl.

In certain embodiments, a 2′-substituted nucleoside comprises a 2′-substituent group selected from F, NH₂, N₃, OCF₃, O—CH₃, O(CH₂)₃NH₂, CH₂—CH═CH₂, O—CH₂—CH═CH₂, OCH₂CH₂OCH₃, O(CH₂)₂SCH₃, O—(CH₂)₂—O—N(Rₘ)(Rₙ), O(CH₂)₂O(CH₂)₂N(CH₃)₂, and N-substituted acetamide (O—CH₂—C(═O)—N(Rₘ)(Rₙ) where each Rₘ and Rₙ is, independently, H, an amino protecting group or substituted or unsubstituted C₁-C₁₀ alkyl.

In certain embodiments, a 2′-substituted nucleoside comprises a sugar moiety comprising a 2′-substituent group selected from F, OCF₃, O—CH₃, OCH₂CH₂OCH₃, O(CH₂)₂SCH₃, O—(CH₂)₂—O—N(CH₃)₂, —O(CH₂)₂O(CH₂)₂N(CH₃)₂, and O—CH₂—C(═O)—N(H)CH₃.

In certain embodiments, a 2′-substituted nucleoside comprises a sugar moiety comprising a 2′-substituent group selected from F, O—CH₃, and OCH₂CH₂OCH₃.

Certain modified sugar moieties comprise a bridging sugar substituent that forms a second ring resulting in a bicyclic sugar moiety. In certain such embodiments, the bicyclic sugar moiety comprises a bridge between the 4′ and the 2′ furanose ring atoms. Examples of such 4′ to 2′ sugar substituents, include, but are not limited to: —[C(Rₐ)(R_{b})]ₙ—, —[C(Rₐ)(R_{b})]ₙ—O—, —C(RₐR_{b})—N(R)—O— or, —C(RₐR_{b})—O—N(R)—; 4′- CH₂-2′, 4′-(CH₂)₂-2′, 4′-(CH₂)₃-2′; 4′-(CH₂)—O-2′ (LNA); 4′-(CH₂)—S-2′; 4′-(CH₂)₂—O-2′ (ENA); 4′-CH(CH₃)—O-2′ (cEt) and 4′-CH(CH₂OCH₃)—O-2′, and analogs thereof (see, e.g., U.S. Pat. No. 7,399,845, issued on Jul. 15, 2008); 4′-C(CH₃)(CH₃)—O-2′ and analogs thereof, (see, e.g., WO2009/006478, published Jan. 8, 2009); 4′-CH₂—N(OCH₃)-2′ and analogs thereof (see, e.g., WO2008/150729, published Dec. 11, 2008); 4′-CH₂—O—N(CH₃)-2′ (see, e.g., US2004/0171570, published Sep. 2, 2004); 4′-CH₂—O—N(R)-2′, and 4′-CH₂—N(R)-0-2′-, wherein each R is, independently, H, a protecting group, or C₁-C₁₂ alkyl; 4′-CH₂—N(R)—O-2′, wherein R is H, C₁-C₁₂ alkyl, or a protecting group (see, U.S. Pat. No. 7,427,672, issued on Sep. 23, 2008); 4′-CH₂—C(H)(CH₃)-2′ (see, e.g., Chattopadhyaya, et al., J. Org. Chem., 2009, 74, 118-134); and 4′-CH₂—C(═CH₂)-2′ and analogs thereof (see, published PCT International Application WO 2008/154401, published on Dec. 8, 2008).

In certain embodiments, such 4′ to 2′ bridges independently comprise from 1 to 4 linked groups independently selected from —[C(Rₐ)(R_{b})]ₙ—, —C(Rₐ)═C(R_{b})—, —C(Rₐ)═N—, —C(═NRₐ)—, —C(═O)—, —C(═S)—, —O—, —Si(Rₐ)₂—, —S(═O)ₓ—, and —N(Rₐ)—;
wherein:
x is 0, 1, or 2;
n is 1, 2, 3, or 4;
each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and
each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Nucleosides comprising bicyclic sugar moieties are referred to as bicyclic nucleosides or BNAs. Bicyclic nucleosides include, but are not limited to, (A) α-L-Methyleneoxy (4′-CH₂—O-2′) BNA, (B) β-D-Methyleneoxy (4′-CH₂—O-2′) BNA (also referred to as locked nucleic acid or LNA), (C) Ethyleneoxy (4′-(CH₂)₂—O-2′) BNA, (D) Aminooxy (4′-CH₂—O—N(R)-2′) BNA, (E) Oxyamino (4′-CH₂—N(R)—O-2′) BNA, (F) Methyl(methyleneoxy) (4′-CH(CH₃)—O-2′) BNA (also referred to as constrained ethyl or cEt), (G) methylene-thio (4′-CH₂—S-2′) BNA, (H) methylene-amino (4′-CH₂—N(R)-2′) BNA, (I) methyl carbocyclic (4′-CH₂—CH(CH₃)-2′) BNA, and (J) propylene carbocyclic (4′-(CH₂)₃-2′) BNA as depicted below.

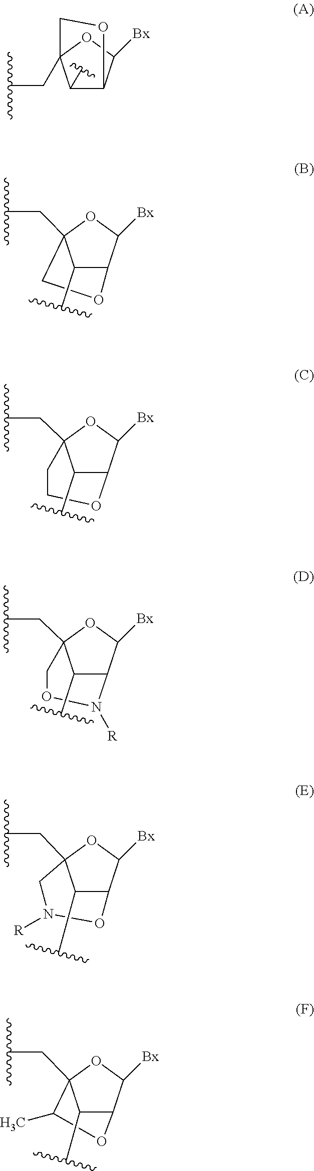

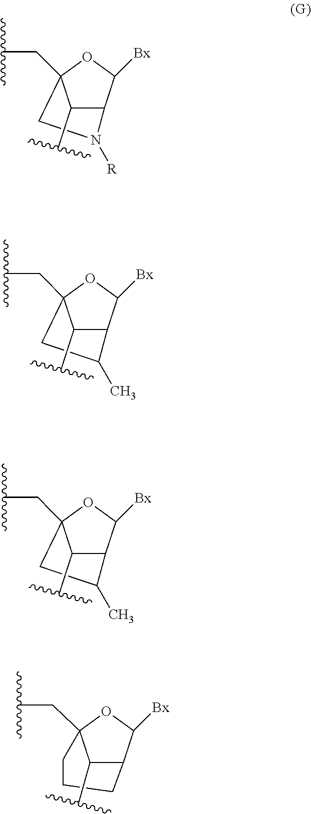

wherein Bx is a nucleobase moiety and R is, independently, H, a protecting group, or C₁-C₁₂ alkyl.

Additional bicyclic sugar moieties are known in the art, for example: Singh et al., Chem. Commun., 1998, 4, 455-456; Koshkin et al., Tetrahedron, 1998, 54, 3607-3630; Wahlestedt et al., Proc. Natl. Acad. Sci. U.S.A, 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J Am. Chem. Soc., 129(26) 8362-8379 (Jul. 4, 2007); Elayadi et al., Curr. Opinion Invens. Drugs, 2001, 2, 558-561; Braasch et al., Chem. Biol., 2001, 8, 1-7; Orum et al., Curr. Opinion Mol. Ther., 2001, 3, 239-243; U.S. Pat. Nos. 7,053,207, 6,268,490, 6,770,748, 6,794,499, 7,034,133, 6,525,191, 6,670,461, and 7,399,845; WO 2004/106356, WO 1994/14226, WO 2005/021570, and WO 2007/134181; U.S. Patent Publication Nos. US2004/0171570, US2007/0287831, and US2008/0039618; U.S. patent Ser. Nos. 12/129,154, 60/989,574, 61/026,995, 61/026,998, 61/056,564, 61/086,231, 61/097,787, and 61/099,844; and PCT International Applications Nos. PCT/US2008/064591, PCT/US2008/066154, and PCT/US2008/068922.

In certain embodiments, bicyclic sugar moieties and nucleosides incorporating such bicyclic sugar moieties are further defined by isomeric configuration. For example, a nucleoside comprising a 4′-2′ methylene-oxy bridge, may be in the α-L configuration or in the β-D configuration. Previously, α-L-methyleneoxy (4′-CH₂—O-2′) bicyclic nucleosides have been incorporated into antisense oligonucleotides that showed antisense activity (Frieden et al., Nucleic Acids Research, 2003, 21, 6365-6372).

In certain embodiments, substituted sugar moieties comprise one or more non-bridging sugar substituent and one or more bridging sugar substituent (e.g., 5′-substituted and 4′-2′ bridged sugars). (see, PCT International Application WO 2007/134181, published on Nov. 22, 2007, wherein LNA is substituted with, for example, a 5′-methyl or a 5′-vinyl group).

In certain embodiments, modified sugar moieties are sugar surrogates. In certain such embodiments, the oxygen atom of the naturally occurring sugar is substituted, e.g., with a sulfur, carbon or nitrogen atom. In certain such embodiments, such modified sugar moiety also comprises bridging and/or non-bridging substituents as described above. For example, certain sugar surrogates comprise a 4′-sulfur atom and a substitution at the 2′-position (see, e.g., published U.S. Patent Application US2005/0130923, published on Jun. 16, 2005) and/or the 5′ position. By way of additional example, carbocyclic bicyclic nucleosides having a 4′-2′ bridge have been described (see, e.g., Freier et al., Nucleic Acids Research, 1997, 25(22), 4429-4443 and Albaek et al., J. Org. Chem., 2006, 71, 7731-7740).

In certain embodiments, sugar surrogates comprise rings having other than 5-atoms. For example, in certain embodiments, a sugar surrogate comprises a morphlino. Morpholino compounds and their use in oligomeric compounds has been reported in numerous patents and published articles (see for example: Braasch et al., Biochemistry, 2002, 41, 4503-4510; and U.S. Pat. Nos. 5,698,685; 5,166,315; 5,185,444; and 5,034,506). As used here, the term “morpholino” means a sugar surrogate having the following structure:

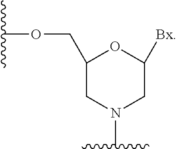

In certain embodiments, morpholinos may be modified, for example by adding or altering various substituent groups from the above morpholino structure. Such sugar surrogates are referred to herein as “modified morpholinos.”

For another example, in certain embodiments, a sugar surrogate comprises a six-membered tetrahydropyran. Such tetrahydropyrans may be further modified or substituted. Nucleosides comprising such modified tetrahydropyrans include, but are not limited to, hexitol nucleic acid (HNA), anitol nucleic acid (ANA), manitol nucleic acid (MNA) (see Leumann, C J. Bioorg. & Med. Chem. (2002) 10:841-854), fluoro HNA (F-HNA), and those compounds having Formula VI:

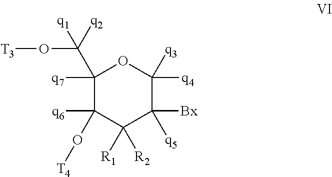

wherein independently for each of said at least one tetrahydropyran nucleoside analog of Formula VI:
Bx is a nucleobase moiety;
T₃ and T₄ are each, independently, an internucleoside linking group linking the tetrahydropyran nucleoside analog to the antisense compound or one of T₃ and T₄ is an internucleoside linking group linking the tetrahydropyran nucleoside analog to the antisense compound and the other of T₃ and T₄ is H, a hydroxyl protecting group, a linked conjugate group, or a 5′ or 3′-terminal group;
q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and
each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, the modified THP nucleosides of Formula VI are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q and q₇ is methyl. In certain embodiments, THP nucleosides of Formula VI are provided wherein one of R₁ and R₂ is F. In certain embodiments, R₁ is fluoro and R₂ is H, R₁ is methoxy and R₂ is H, and R₁ is methoxyethoxy and R₂ is H.

Many other bicyclo and tricyclo sugar surrogate ring systems are also known in the art that can be used to modify nucleosides for incorporation into antisense compounds (see, e.g., review article: Leumann, J. C, Bioorganic & Medicinal Chemistry, 2002, 10, 841-854).

Combinations of modifications are also provided without limitation, such as 2′-F-5′-methyl substituted nucleosides (see PCT International Application WO 2008/101157 Published on Aug. 21, 2008 for other disclosed 5′, 2′-bis substituted nucleosides) and replacement of the ribosyl ring oxygen atom with S and further substitution at the 2′-position (see published U.S. Patent Application US2005-0130923, published on Jun. 16, 2005) or alternatively 5′-substitution of a bicyclic nucleic acid (see PCT International Application WO 2007/134181, published on Nov. 22, 2007 wherein a 4′-CH₂—O-2′ bicyclic nucleoside is further substituted at the 5′ position with a 5′-methyl or a 5′-vinyl group). The synthesis and preparation of carbocyclic bicyclic nucleosides along with their oligomerization and biochemical studies have also been described (see, e.g., Srivastava et al., J. Am. Chem. Soc. 2007, 129(26), 8362-8379).

In certain embodiments, the present disclosure provides oligonucleotides comprising modified nucleosides. Those modified nucleotides may include modified sugars, modified nucleobases, and/or modified linkages. The specific modifications are selected such that the resulting oligonucleotides possess desirable characteristics. In certain embodiments, oligonucleotides comprise one or more RNA-like nucleosides. In certain embodiments, oligonucleotides comprise one or more DNA-like nucleotides.

### Certain Nucleobase Modifications

In certain embodiments, nucleosides of the present disclosure comprise one or more unmodified nucleobases. In certain embodiments, nucleosides of the present disclosure comprise one or more modified nucleobases.

In certain embodiments, modified nucleobases are selected from: universal bases, hydrophobic bases, promiscuous bases, size-expanded bases, and fluorinated bases as defined herein. 5-substituted pyrimidines, 6-azapyrimidines and N-2, N-6 and 0-6 substituted purines, including 2-aminopropyladenine, 5-propynyluracil; 5-propynylcytosine; 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine, 3-deazaguanine and 3-deazaadenine, universal bases, hydrophobic bases, promiscuous bases, size-expanded bases, and fluorinated bases as defined herein. Further modified nucleobases include tricyclic pyrimidines such as phenoxazine cytidine([5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, Kroschwitz, J. I., Ed., John Wiley & Sons, 1990, 858-859; those disclosed by Englisch et al., Angewandte Chemie, International Edition, 1991, 30, 613; and those disclosed by Sanghvi, Y. S., Chapter 15, Antisense Research and Applications, Crooke, S. T. and Lebleu, B., Eds., CRC Press, 1993, 273-288.

Representative United States patents that teach the preparation of certain of the above noted modified nucleobases as well as other modified nucleobases include without limitation, U.S. Pat. Nos. 3,687,808; 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121; 5,596,091; 5,614,617; 5,645,985; 5,681,941; 5,750,692; 5,763,588; 5,830,653 and 6,005,096, certain of which are commonly owned with the instant application, and each of which is herein incorporated by reference in its entirety.

### Certain Internucleoside Linkages

In certain embodiments, the present disclosure provides oligonucleotides comprising linked nucleosides. In such embodiments, nucleosides may be linked together using any internucleoside linkage. The two main classes of internucleoside linking groups are defined by the presence or absence of a phosphorus atom. Representative phosphorus containing internucleoside linkages include, but are not limited to, phosphodiesters (PO), phosphotriesters, methylphosphonates, phosphoramidate, and phosphorothioates (PS). Representative non-phosphorus containing internucleoside linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N′-dimethylhydrazine (—CH₂—N(CH₃)—N(CH₃)—). Modified linkages, compared to natural phosphodiester linkages, can be used to alter, typically increase, nuclease resistance of the oligonucleotide. In certain embodiments, internucleoside linkages having a chiral atom can be prepared as a racemic mixture, or as separate enantiomers. Representative chiral linkages include, but are not limited to, alkylphosphonates and phosphorothioates. Methods of preparation of phosphorous-containing and non-phosphorous-containing internucleoside linkages are well known to those skilled in the art.

The oligonucleotides described herein contain one or more asymmetric centers and thus give rise to enantiomers, diastereomers, and other stereoisomeric configurations that may be defined, in terms of absolute stereochemistry, as (R) or (S), ^{—} or ┌ such as for sugar anomers, or as (D) or (L) such as for amino acids etc. Included in the antisense compounds provided herein are all such possible isomers, as well as their racemic and optically pure forms.

Neutral internucleoside linkages include without limitation, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

### Certain Motifs

In certain embodiments, antisense oligonucleotides comprise one or more modified nucleoside (e.g., nucleoside comprising a modified sugar and/or modified nucleobase) and/or one or more modified internucleoside linkage. The pattern of such modifications on an oligonucleotide is referred to herein as a motif. In certain embodiments, sugar, nucleobase, and linkage motifs are independent of one another.

### Certain Sugar Motifs

In certain embodiments, oligonucleotides comprise one or more type of modified sugar moieties and/or naturally occurring sugar moieties arranged along an oligonucleotide or region thereof in a defined pattern or sugar modification motif Such motifs may include any of the sugar modifications discussed herein and/or other known sugar modifications.

In certain embodiments, the oligonucleotides comprise or consist of a region having a gapmer sugar motif, which comprises two external regions or “wings” and a central or internal region or “gap.” The three regions of a gapmer sugar motif (the 5′-wing, the gap, and the 3′-wing) form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are closest to the gap (the 3′-most nucleoside of the 5′-wing and the 5′-most nucleoside of the 3′-wing) differ from the sugar moiety of the neighboring gap nucleosides, thus defining the boundary between the wings and the gap. In certain embodiments, the sugar moieties within the gap are the same as one another. In certain embodiments, the gap includes one or more nucleoside having a sugar moiety that differs from the sugar moiety of one or more other nucleosides of the gap. In certain embodiments, the sugar motifs of the two wings are the same as one another (symmetric sugar gapmer). In certain embodiments, the sugar motifs of the 5′-wing differs from the sugar motif of the 3′-wing (asymmetric sugar gapmer).

### Certain 5′-Wings

In certain embodiments, the 5′-wing of a gapmer consists of 1 to 8 linked nucleosides. In certain embodiments, the 5′-wing of a gapmer consists of 1 to 7 linked nucleosides. In certain embodiments, the 5′-wing of a gapmer consists of 1 to 6 linked nucleosides. In certain embodiments, the 5′-wing of a gapmer consists of 1 to 5 linked nucleosides. In certain embodiments, the 5′-wing of a gapmer consists of 2 to 5 linked nucleosides. In certain embodiments, the 5′-wing of a gapmer consists of 3 to 5 linked nucleosides. In certain embodiments, the 5′-wing of a gapmer consists of 4 or 5 linked nucleosides. In certain embodiments, the 5′-wing of a gapmer consists of 1 to 4 linked nucleosides. In certain embodiments, the 5′-wing of a gapmer consists of 1 to 3 linked nucleosides. In certain embodiments, the 5′-wing of a gapmer consists of 1 or 2 linked nucleosides. In certain embodiments, the 5′-wing of a gapmer consists of 2 to 4 linked nucleosides. In certain embodiments, the 5′-wing of a gapmer consists of 2 or 3 linked nucleosides. In certain embodiments, the 5′-wing of a gapmer consists of 3 or 4 linked nucleosides. In certain embodiments, the 5′-wing of a gapmer consists of 1 nucleoside. In certain embodiments, the 5′-wing of a gapmer consists of 2 linked nucleosides. In certain embodiments, the 5′-wing of a gapmer consists of 3 linked nucleosides. In certain embodiments, the 5′-wing of a gapmer consists of 4 linked nucleosides. In certain embodiments, the 5′-wing of a gapmer consists of 5 linked nucleosides. In certain embodiments, the 5′-wing of a gapmer consists of 6 linked nucleosides.

In certain embodiments, the 5′-wing of a gapmer comprises at least one bicyclic nucleoside. In certain embodiments, the 5′-wing of a gapmer comprises at least two bicyclic nucleosides. In certain embodiments, the 5′-wing of a gapmer comprises at least three bicyclic nucleosides. In certain embodiments, the 5′-wing of a gapmer comprises at least four bicyclic nucleosides. In certain embodiments, the 5′-wing of a gapmer comprises at least one constrained ethyl nucleoside. In certain embodiments, the 5′-wing of a gapmer comprises at least one LNA nucleoside. In certain embodiments, each nucleoside of the 5′-wing of a gapmer is a bicyclic nucleoside. In certain embodiments, each nucleoside of the 5′-wing of a gapmer is a constrained ethyl nucleoside. In certain embodiments, each nucleoside of the 5′-wing of a gapmer is a LNA nucleoside.

In certain embodiments, the 5′-wing of a gapmer comprises at least one non-bicyclic modified nucleoside. In certain embodiments, the 5′-wing of a gapmer comprises at least one 2′-substituted nucleoside. In certain embodiments, the 5′-wing of a gapmer comprises at least one 2′-MOE nucleoside. In certain embodiments, the 5′-wing of a gapmer comprises at least one 2′-OMe nucleoside. In certain embodiments, each nucleoside of the 5′-wing of a gapmer is a non-bicyclic modified nucleoside. In certain embodiments, each nucleoside of the 5′-wing of a gapmer is a 2′-substituted nucleoside. In certain embodiments, each nucleoside of the 5′-wing of a gapmer is a 2′-MOE nucleoside. In certain embodiments, each nucleoside of the 5′-wing of a gapmer is a 2′-OMe nucleoside.

In certain embodiments, the 5′-wing of a gapmer comprises at least one 2′-deoxynucleoside. In certain embodiments, each nucleoside of the 5′-wing of a gapmer is a 2′-deoxynucleoside. In a certain embodiments, the 5′-wing of a gapmer comprises at least one ribonucleoside. In certain embodiments, each nucleoside of the 5′-wing of a gapmer is a ribonucleoside. In certain embodiments, one, more than one, or each of the nucleosides of the 5′-wing is an RNA-like nucleoside.

In certain embodiments, the 5′-wing of a gapmer comprises at least one bicyclic nucleoside and at least one non-bicyclic modified nucleoside. In certain embodiments, the 5′-wing of a gapmer comprises at least one bicyclic nucleoside and at least one 2′-substituted nucleoside. In certain embodiments, the 5′-wing of a gapmer comprises at least one bicyclic nucleoside and at least one 2′-MOE nucleoside. In certain embodiments, the 5′-wing of a gapmer comprises at least one bicyclic nucleoside and at least one 2′-OMe nucleoside. In certain embodiments, the 5′-wing of a gapmer comprises at least one bicyclic nucleoside and at least one 2′-deoxynucleoside.

In certain embodiments, the 5′-wing of a gapmer comprises at least one constrained ethyl nucleoside and at least one non-bicyclic modified nucleoside. In certain embodiments, the 5′-wing of a gapmer comprises at least one constrained ethyl nucleoside and at least one 2′-substituted nucleoside. In certain embodiments, the 5′-wing of a gapmer comprises at least one constrained ethyl nucleoside and at least one 2′-MOE nucleoside.

In certain embodiments, the 5′-wing of a gapmer comprises at least one constrained ethyl nucleoside and at least one 2′-OMe nucleoside. In certain embodiments, the 5′-wing of a gapmer comprises at least one constrained ethyl nucleoside and at least one 2′-deoxynucleoside.

### Certain 3′-Wings

In certain embodiments, the 3′-wing of a gapmer consists of 1 to 8 linked nucleosides. In certain embodiments, the 3′-wing of a gapmer consists of 1 to 7 linked nucleosides. In certain embodiments, the 3′-wing of a gapmer consists of 1 to 6 linked nucleosides. In certain embodiments, the 3′-wing of a gapmer consists of 1 to 5 linked nucleosides. In certain embodiments, the 3′-wing of a gapmer consists of 2 to 5 linked nucleosides. In certain embodiments, the 3′-wing of a gapmer consists of 3 to 5 linked nucleosides. In certain embodiments, the 3′-wing of a gapmer consists of 4 or 5 linked nucleosides. In certain embodiments, the 3′-wing of a gapmer consists of 1 to 4 linked nucleosides. In certain embodiments, the 3′-wing of a gapmer consists of 1 to 3 linked nucleosides. In certain embodiments, the 3′-wing of a gapmer consists of 1 or 2 linked nucleosides. In certain embodiments, the 3′-wing of a gapmer consists of 2 to 4 linked nucleosides. In certain embodiments, the 3′-wing of a gapmer consists of 2 or 3 linked nucleosides.

In certain embodiments, the 3′-wing of a gapmer consists of 3 or 4 linked nucleosides. In certain embodiments, the 3′-wing of a gapmer consists of 1 nucleoside. In certain embodiments, the 3′-wing of a gapmer consists of 2 linked nucleosides. In certain embodiments, the 3′-wing of a gapmer consists of 3 linked nucleosides. In certain embodiments, the 3′-wing of a gapmer consists of 4 linked nucleosides. In certain embodiments, the 3′-wing of a gapmer consists of 5 linked nucleosides. In certain embodiments, the 3′-wing of a gapmer consists of 6 linked nucleosides.

In certain embodiments, the 3′-wing of a gapmer comprises at least one bicyclic nucleoside. In certain embodiments, the 3′-wing of a gapmer comprises at least one constrained ethyl nucleoside. In certain embodiments, the 3′-wing of a gapmer comprises at least one LNA nucleoside. In certain embodiments, each nucleoside of the 3′-wing of a gapmer is a bicyclic nucleoside. In certain embodiments, each nucleoside of the 3′-wing of a gapmer is a constrained ethyl nucleoside. In certain embodiments, each nucleoside of the 3′-wing of a gapmer is a LNA nucleoside.

In certain embodiments, the 3′-wing of a gapmer comprises at least one non-bicyclic modified nucleoside. In certain embodiments, the 3′-wing of a gapmer comprises at least two non-bicyclic modified nucleosides. In certain embodiments, the 3′-wing of a gapmer comprises at least three non-bicyclic modified nucleosides. In certain embodiments, the 3′-wing of a gapmer comprises at least four non-bicyclic modified nucleosides. In certain embodiments, the 3′-wing of a gapmer comprises at least one 2′-substituted nucleoside. In certain embodiments, the 3′-wing of a gapmer comprises at least one 2′-MOE nucleoside. In certain embodiments, the 3′-wing of a gapmer comprises at least one 2′-OMe nucleoside. In certain embodiments, each nucleoside of the 3′-wing of a gapmer is a non-bicyclic modified nucleoside. In certain embodiments, each nucleoside of the 3′-wing of a gapmer is a 2′-substituted nucleoside. In certain embodiments, each nucleoside of the 3′-wing of a gapmer is a 2′-MOE nucleoside. In certain embodiments, each nucleoside of the 3′-wing of a gapmer is a 2′-OMe nucleoside.

In certain embodiments, the 3′-wing of a gapmer comprises at least one 2′-deoxynucleoside. In certain embodiments, each nucleoside of the 3′-wing of a gapmer is a 2′-deoxynucleoside. In a certain embodiments, the 3′-wing of a gapmer comprises at least one ribonucleoside. In certain embodiments, each nucleoside of the 3′-wing of a gapmer is a ribonucleoside. In certain embodiments, one, more than one, or each of the nucleosides of the 5′-wing is an RNA-like nucleoside.

In certain embodiments, the 3′-wing of a gapmer comprises at least one bicyclic nucleoside and at least one non-bicyclic modified nucleoside. In certain embodiments, the 3′-wing of a gapmer comprises at least one bicyclic nucleoside and at least one 2′-substituted nucleoside. In certain embodiments, the 3′-wing of a gapmer comprises at least one bicyclic nucleoside and at least one 2′-MOE nucleoside. In certain embodiments, the 3′-wing of a gapmer comprises at least one bicyclic nucleoside and at least one 2′-OMe nucleoside. In certain embodiments, the 3′-wing of a gapmer comprises at least one bicyclic nucleoside and at least one 2′-deoxynucleoside.

In certain embodiments, the 3′-wing of a gapmer comprises at least one constrained ethyl nucleoside and at least one non-bicyclic modified nucleoside. In certain embodiments, the 3′-wing of a gapmer comprises at least one constrained ethyl nucleoside and at least one 2′-substituted nucleoside. In certain embodiments, the 3′-wing of a gapmer comprises at least one constrained ethyl nucleoside and at least one 2′-MOE nucleoside. In certain embodiments, the 3′-wing of a gapmer comprises at least one constrained ethyl nucleoside and at least one 2′-OMe nucleoside. In certain embodiments, the 3′-wing of a gapmer comprises at least one constrained ethyl nucleoside and at least one 2′-deoxynucleoside.

In certain embodiments, the 3′-wing of a gapmer comprises at least one LNA nucleoside and at least one non-bicyclic modified nucleoside. In certain embodiments, the 3′-wing of a gapmer comprises at least one LNA nucleoside and at least one 2′-substituted nucleoside. In certain embodiments, the 3′-wing of a gapmer comprises at least one LNA nucleoside and at least one 2′-MOE nucleoside. In certain embodiments, the 3′-wing of a gapmer comprises at least one LNA nucleoside and at least one 2′-OMe nucleoside. In certain embodiments, the 3′-wing of a gapmer comprises at least one LNA nucleoside and at least one 2′-deoxynucleoside.

In certain embodiments, the 3′-wing of a gapmer comprises at least one bicyclic nucleoside, at least one non-bicyclic modified nucleoside, and at least one 2′-deoxynucleoside. In certain embodiments, the 3′-wing of a gapmer comprises at least one constrained ethyl nucleoside, at least one non-bicyclic modified nucleoside, and at least one 2′-deoxynucleoside. In certain embodiments, the 3′-wing of a gapmer comprises at least one LNA nucleoside, at least one non-bicyclic modified nucleoside, and at least one 2′-deoxynucleoside.

In certain embodiments, the 3′-wing of a gapmer comprises at least one bicyclic nucleoside, at least one 2′-substituted nucleoside, and at least one 2′-deoxynucleoside. In certain embodiments, the 3′-wing of a gapmer comprises at least one constrained ethyl nucleoside, at least one 2′-substituted nucleoside, and at least one 2′-deoxynucleoside. In certain embodiments, the 3′-wing of a gapmer comprises at least one LNA nucleoside, at least one 2′-substituted nucleoside, and at least one 2′-deoxynucleoside.

In certain embodiments, the 3′-wing of a gapmer comprises at least one bicyclic nucleoside, at least one 2′-MOE nucleoside, and at least one 2′-deoxynucleoside. In certain embodiments, the 3′-wing of a gapmer comprises at least one constrained ethyl nucleoside, at least one 2′-MOE nucleoside, and at least one 2′-deoxynucleoside. In certain embodiments, the 3′-wing of a gapmer comprises at least one LNA nucleoside, at least one 2′-MOE nucleoside, and at least one 2′-deoxynucleoside.

In certain embodiments, the 3′-wing of a gapmer comprises at least one bicyclic nucleoside, at least one 2′-OMe nucleoside, and at least one 2′-deoxynucleoside. In certain embodiments, the 3′-wing of a gapmer comprises at least one constrained ethyl nucleoside, at least one 2′-OMe nucleoside, and at least one 2′-deoxynucleoside. In certain embodiments, the 3′-wing of a gapmer comprises at least one LNA nucleoside, at least one 2′-OMe nucleoside, and at least one 2′-deoxynucleoside.

### Certain Central Regions (Gaps)

In certain embodiments, the gap of a gapmer consists of 6 to 20 linked nucleosides. In certain embodiments, the gap of a gapmer consists of 6 to 15 linked nucleosides. In certain embodiments, the gap of a gapmer consists of 6 to 12 linked nucleosides. In certain embodiments, the gap of a gapmer consists of 6 to 10 linked nucleosides. In certain embodiments, the gap of a gapmer consists of 6 to 9 linked nucleosides. In certain embodiments, the gap of a gapmer consists of 6 to 8 linked nucleosides. In certain embodiments, the gap of a gapmer consists of 6 or 7 linked nucleosides. In certain embodiments, the gap of a gapmer consists of 7 to 10 linked nucleosides. In certain embodiments, the gap of a gapmer consists of 7 to 9 linked nucleosides. In certain embodiments, the gap of a gapmer consists of 7 or 8 linked nucleosides. In certain embodiments, the gap of a gapmer consists of 8 to 10 linked nucleosides. In certain embodiments, the gap of a gapmer consists of 8 or 9 linked nucleosides. In certain embodiments, the gap of a gapmer consists of 6 linked nucleosides. In certain embodiments, the gap of a gapmer consists of 7 linked nucleosides. In certain embodiments, the gap of a gapmer consists of 8 linked nucleosides. In certain embodiments, the gap of a gapmer consists of 9 linked nucleosides. In certain embodiments, the gap of a gapmer consists of 10 linked nucleosides. In certain embodiments, the gap of a gapmer consists of 11 linked nucleosides. In certain embodiments, the gap of a gapmer consists of 12 linked nucleosides.

In certain embodiments, each nucleoside of the gap of a gapmer is a 2′-deoxynucleoside. In certain embodiments, the gap comprises one or more modified nucleosides. In certain embodiments, each nucleoside of the gap of a gapmer is a 2′-deoxynucleoside or is a modified nucleoside that is “DNA-like.” In such embodiments, “DNA-like” means that the nucleoside has similar characteristics to DNA, such that a duplex comprising the gapmer and an RNA molecule is capable of activating RNase H. For example, under certain conditions, 2′-(ara)-F have been shown to support RNase H activation, and thus is DNA-like. In certain embodiments, one or more nucleosides of the gap of a gapmer is not a 2′-deoxynucleoside and is not DNA-like. In certain such embodiments, the gapmer nonetheless supports RNase H activation (e.g., by virtue of the number or placement of the non-DNA nucleosides).

In certain embodiments, gaps comprise a stretch of unmodified 2′-deoxynucleoside interrupted by one or more modified nucleosides, thus resulting in three sub-regions (two stretches of one or more 2′-deoxynucleosides and a stretch of one or more interrupting modified nucleosides). In certain embodiments, no stretch of unmodified 2′-deoxynucleosides is longer than 5, 6, or 7 nucleosides. In certain embodiments, such short stretches is achieved by using short gap regions. In certain embodiments, short stretches are achieved by interrupting a longer gap region.

In certain embodiments, the gap comprises one or more modified nucleosides. In certain embodiments, the gap comprises one or more modified nucleosides selected from among cEt, FHNA, LNA, and 2-thio-thymidine. In certain embodiments, the gap comprises one modified nucleoside. In certain embodiments, the gap comprises a 5′-substituted sugar moiety selected from among 5′-Me, and 5′-(R)-Me. In certain embodiments, the gap comprises two modified nucleosides. In certain embodiments, the gap comprises three modified nucleosides. In certain embodiments, the gap comprises four modified nucleosides. In certain embodiments, the gap comprises two or more modified nucleosides and each modified nucleoside is the same. In certain embodiments, the gap comprises two or more modified nucleosides and each modified nucleoside is different.

In certain embodiments, the gap comprises one or more modified linkages. In certain embodiments, the gap comprises one or more methyl phosphonate linkages. In certain embodiments the gap comprises two or more modified linkages. In certain embodiments, the gap comprises one or more modified linkages and one or more modified nucleosides. In certain embodiments, the gap comprises one modified linkage and one modified nucleoside. In certain embodiments, the gap comprises two modified linkages and two or more modified nucleosides.

### Certain Internucleoside Linkage Motifs

In certain embodiments, oligonucleotides comprise modified internucleoside linkages arranged along the oligonucleotide or region thereof in a defined pattern or modified internucleoside linkage motif. In certain embodiments, oligonucleotides comprise a region having an alternating internucleoside linkage motif. In certain embodiments, oligonucleotides of the present disclosure comprise a region of uniformly modified internucleoside linkages. In certain such embodiments, the oligonucleotide comprises a region that is uniformly linked by phosphorothioate internucleoside linkages. In certain embodiments, the oligonucleotide is uniformly linked by phosphorothioate internucleoside linkages. In certain embodiments, each internucleoside linkage of the oligonucleotide is selected from phosphodiester and phosphorothioate. In certain embodiments, each internucleoside linkage of the oligonucleotide is selected from phosphodiester and phosphorothioate and at least one internucleoside linkage is phosphorothioate.

In certain embodiments, the oligonucleotide comprises at least 6 phosphorothioate internucleoside linkages. In certain embodiments, the oligonucleotide comprises at least 7 phosphorothioate internucleoside linkages. In certain embodiments, the oligonucleotide comprises at least 8 phosphorothioate internucleoside linkages. In certain embodiments, the oligonucleotide comprises at least 9 phosphorothioate internucleoside linkages. In certain embodiments, the oligonucleotide comprises at least 10 phosphorothioate internucleoside linkages. In certain embodiments, the oligonucleotide comprises at least 11 phosphorothioate internucleoside linkages. In certain embodiments, the oligonucleotide comprises at least 12 phosphorothioate internucleoside linkages. In certain embodiments, the oligonucleotide comprises at least 13 phosphorothioate internucleoside linkages. In certain embodiments, the oligonucleotide comprises at least 14 phosphorothioate internucleoside linkages.

In certain embodiments, the oligonucleotide comprises at least one block of at least 6 consecutive phosphorothioate internucleoside linkages. In certain embodiments, the oligonucleotide comprises at least one block of at least 7 consecutive phosphorothioate internucleoside linkages. In certain embodiments, the oligonucleotide comprises at least one block of at least 8 consecutive phosphorothioate internucleoside linkages. In certain embodiments, the oligonucleotide comprises at least one block of at least 9 consecutive phosphorothioate internucleoside linkages. In certain embodiments, the oligonucleotide comprises at least one block of at least 10 consecutive phosphorothioate internucleoside linkages. In certain embodiments, the oligonucleotide comprises at least block of at least one 12 consecutive phosphorothioate internucleoside linkages. In certain such embodiments, at least one such block is located at the 3′ end of the oligonucleotide. In certain such embodiments, at least one such block is located within 3 nucleosides of the 3′ end of the oligonucleotide. In certain embodiments, the oligonucleotide comprises less than 15 phosphorothioate internucleoside linkages. In certain embodiments, the oligonucleotide comprises less than 14 phosphoro-thioate internucleoside linkages. In certain embodiments, the oligonucleotide comprises less than 13 phosphorothioate internucleoside linkages. In certain embodiments, the oligonucleotide comprises less than 12 phosphorothioate internucleoside linkages. In certain embodiments, the oligonucleotide comprises less than 11 phosphorothioate internucleoside linkages. In certain embodiments, the oligonucleotide comprises less than 10 phosphorothioate internucleoside linkages. In certain embodiments, the oligonucleotide comprises less than 9 phosphorothioate internucleoside linkages. In certain embodiments, the oligonucleotide comprises less than 8 phosphorothioate internucleoside linkages. In certain embodiments, the oligonucleotide comprises less than 7 phosphorothioate internucleoside linkages. In certain embodiments, the oligonucleotide comprises less than 6 phosphorothioate internucleoside linkages. In certain embodiments, the oligonucleotide comprises less than 5 phosphorothioate internucleoside linkages.

### Certain Nucleobase Modification Motifs

In certain embodiments, oligonucleotides comprise chemical modifications to nucleobases arranged along the oligonucleotide or region thereof in a defined pattern or nucleobases modification motif. In certain such embodiments, nucleobase modifications are arranged in a gapped motif. In certain embodiments, nucleobase modifications are arranged in an alternating motif. In certain embodiments, each nucleobase is modified. In certain embodiments, none of the nucleobases is chemically modified.

In certain embodiments, oligonucleotides comprise a block of modified nucleobases. In certain such embodiments, the block is at the 3′-end of the oligonucleotide. In certain embodiments the block is within 3 nucleotides of the 3′-end of the oligonucleotide. In certain such embodiments, the block is at the 5′-end of the oligonucleotide. In certain embodiments the block is within 3 nucleotides of the 5′-end of the oligonucleotide.

In certain embodiments, nucleobase modifications are a function of the natural base at a particular position of an oligonucleotide. For example, in certain embodiments each purine or each pyrimidine in an oligonucleotide is modified. In certain embodiments, each adenine is modified. In certain embodiments, each guanine is modified. In certain embodiments, each thymine is modified. In certain embodiments, each cytosine is modified. In certain embodiments, each uracil is modified.

In certain embodiments, some, all, or none of the cytosine moieties in an oligonucleotide are 5-methyl cytosine moieties. Herein, 5-methyl cytosine is not a “modified nucleobase.” Accordingly, unless otherwise indicated, unmodified nucleobases include both cytosine residues having a 5-methyl and those lacking a 5 methyl. In certain embodiments, the methylation state of all or some cytosine nucleobases is specified.

In certain embodiments, chemical modifications to nucleobases comprise attachment of certain conjugate groups to nucleobases. In certain embodiments, each purine or each pyrimidine in an oligonucleotide may be optionally modified to comprise a conjugate group.

### Certain Overall Lengths

In certain embodiments, the present disclosure provides oligonucleotides of any of a variety of ranges of lengths. In certain embodiments, oligonucleotides consist of X to Y linked nucleosides, where X represents the fewest number of nucleosides in the range and Y represents the largest number of nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X≤Y. For example, in certain embodiments, the oligonucleotide may consist of 8 to 9, 8 to 10, 8 to 11, 8 to 12, 8 to 13, 8 to 14, 8 to 15, 8 to 16, 8 to 17, 8 to 18, 8 to 19, 8 to 20, 8 to 21, 8 to 22, 8 to 23, 8 to 24, 8 to 25, 8 to 26, 8 to 27, 8 to 28, 8 to 29, 8 to 30, 9 to 10, 9 to 11, 9 to 12, 9 to 13, 9 to 14, 9 to 15, 9 to 16, 9 to 17, 9 to 18, 9 to 19, 9 to 20, 9 to 21, 9 to 22, 9 to 23, 9 to 24, 9 to 25, 9 to 26, 9 to 27, 9 to 28, 9 to 29, 9 to 30, 10 to 11, 10 to 12, 10 to 13, 10 to 14, 10 to 15, 10 to 16, 10 to 17, 10 to 18, 10 to 19, 10 to 20, 10 to 21, 10 to 22, 10 to 23, 10 to 24, 10 to 25, 10 to 26, 10 to 27, 10 to 28, 10 to 29, 10 to 30, 11 to 12, 11 to 13, 11 to 14, 11 to 15, 11 to 16, 11 to 17, 11 to 18, 11 to 19, 11 to 20, 11 to 21, 11 to 22, 11 to 23, 11 to 24, 11 to 25, 11 to 26, 11 to 27, 11 to 28, 11 to 29, 11 to 30, 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides. In embodiments where the number of nucleosides of an oligonucleotide of a compound is limited, whether to a range or to a specific number, the compound may, nonetheless further comprise additional other substituents. For example, an oligonucleotide comprising 8-30 nucleosides excludes oligonucleotides having 31 nucleosides, but, unless otherwise indicated, such an oligonucleotide may further comprise, for example one or more conjugate groups, terminal groups, or other substituents.

Further, where an oligonucleotide is described by an overall length range and by regions having specified lengths, and where the sum of specified lengths of the regions is less than the upper limit of the overall length range, the oligonucleotide may have additional nucleosides, beyond those of the specified regions, provided that the total number of nucleosides does not exceed the upper limit of the overall length range.

### Certain Antisense Oligonucleotide Chemistry Motifs

In certain embodiments, the chemical structural features of antisense oligonucleotides are characterized by their sugar motif, internucleoside linkage motif, nucleobase modification motif and overall length. In certain embodiments, such parameters are each independent of one another. Thus, each internucleoside linkage of an oligonucleotide having a gapmer sugar motif may be modified or unmodified and may or may not follow the gapmer modification pattern of the sugar modifications. Thus, the internucleoside linkages within the wing regions of a sugar-gapmer may be the same or different from one another and may be the same or different from the internucleoside linkages of the gap region. Likewise, such sugar-gapmer oligonucleotides may comprise one or more modified nucleobase independent of the gapmer pattern of the sugar modifications. One of skill in the art will appreciate that such motifs may be combined to create a variety of oligonucleotides.

In certain embodiments, the selection of internucleoside linkage and nucleoside modification are not independent of one another.

a. Certain Sequences and Targets

In certain embodiments, the invention provides antisense oligonucleotides having a sequence complementary to a target nucleic acid. Such antisense compounds are capable of hybridizing to a target nucleic acid, resulting in at least one antisense activity. In certain embodiments, antisense compounds specifically hybridize to one or more target nucleic acid. In certain embodiments, a specifically hybridizing antisense compound has a nucleobase sequence comprising a region having sufficient complementarity to a target nucleic acid to allow hybridization and result in antisense activity and insufficient complementarity to any non-target so as to avoid or reduce non-specific hybridization to non-target nucleic acid sequences under conditions in which specific hybridization is desired (e.g., under physiological conditions for in vivo or therapeutic uses, and under conditions in which assays are performed in the case of in vitro assays). In certain embodiments, oligonucleotides are selective between a target and non-target, even though both target and non-target comprise the target sequence. In such embodiments, selectivity may result from relative accessibility of the target region of one nucleic acid molecule compared to the other.

In certain embodiments, the present disclosure provides antisense compounds comprising oligonucleotides that are fully complementary to the target nucleic acid over the entire length of the oligonucleotide. In certain embodiments, oligonucleotides are 99% complementary to the target nucleic acid. In certain embodiments, oligonucleotides are 95% complementary to the target nucleic acid. In certain embodiments, such oligonucleotides are 90% complementary to the target nucleic acid.

In certain embodiments, such oligonucleotides are 85% complementary to the target nucleic acid. In certain embodiments, such oligonucleotides are 80% complementary to the target nucleic acid. In certain embodiments, an antisense compound comprises a region that is fully complementary to a target nucleic acid and is at least 80% complementary to the target nucleic acid over the entire length of the oligonucleotide. In certain such embodiments, the region of full complementarity is from 6 to 14 nucleobases in length.

In certain embodiments, oligonucleotides comprise a hybridizing region and a terminal region. In certain such embodiments, the hybridizing region consists of 12-30 linked nucleosides and is fully complementary to the target nucleic acid. In certain embodiments, the hybridizing region includes one mismatch relative to the target nucleic acid. In certain embodiments, the hybridizing region includes two mismatches relative to the target nucleic acid. In certain embodiments, the hybridizing region includes three mismatches relative to the target nucleic acid. In certain embodiments, the terminal region consists of 1-4 terminal nucleosides. In certain embodiments, the terminal nucleosides are at the 3′ end. In certain embodiments, one or more of the terminal nucleosides are not complementary to the target nucleic acid.

Antisense mechanisms include any mechanism involving the hybridization of an oligonucleotide with target nucleic acid, wherein the hybridization results in a biological effect. In certain embodiments, such hybridization results in either target nucleic acid degradation or occupancy with concomitant inhibition or stimulation of the cellular machinery involving, for example, translation, transcription, or splicing of the target nucleic acid.

One type of antisense mechanism involving degradation of target RNA is RNase H mediated antisense. RNase H is a cellular endonuclease which cleaves the RNA strand of an RNA:DNA duplex. It is known in the art that single-stranded antisense compounds which are “DNA-like” elicit RNase H activity in mammalian cells. Activation of RNase H, therefore, results in cleavage of the RNA target, thereby greatly enhancing the efficiency of DNA-like oligonucleotide-mediated inhibition of gene expression.

In certain embodiments, a conjugate group comprises a cleavable moiety. In certain embodiments, a conjugate group comprises one or more cleavable bond. In certain embodiments, a conjugate group comprises a linker. In certain embodiments, a linker comprises a protein binding moiety. In certain embodiments, a conjugate group comprises a cell-targeting moiety (also referred to as a cell-targeting group). In certain embodiments a cell-targeting moiety comprises a branching group. In certain embodiments, a cell-targeting moiety comprises one or more tethers. In certain embodiments, a cell-targeting moiety comprises a carbohydrate or carbohydrate cluster.

b. Certain Connecting Groups

In certain embodiments, one or more conjugates are attached to an oligomeric compound through a connecting group. In certain embodiments, a connecting group includes a tether or a portion of a tether. In certain embodiments, a connecting group includes a branching group or a portion of a branching group. In certain embodiments, a connecting group includes a linking group or a portion of a linking group. In certain embodiments, a connecting group includes a cleavable moiety or a portion of a cleavable moiety. In certain embodiments, a connecting group includes a tether, a branching group, and/or a linking group or a portion of a tether, a branching group, and/or a linking group.

In certain embodiments, a connecting group includes a tether and a branching group. In certain embodiments, a connecting group includes a portion of a tether and branching group. In certain embodiments, a connecting group includes a tether and portion of a branching group. In certain embodiments, a connecting group includes or a portion of a tether and portion of a branching group.

In certain embodiments, a connecting group includes a tether and a linking group. In certain embodiments, a connecting group includes a portion of a tether and linking group. In certain embodiments, a connecting group includes a tether and portion of a linking group. In certain embodiments, a connecting group includes a portion of a tether and portion of a linking group.

In certain embodiments, a connecting group includes a branching group and a linking group. In certain embodiments, a connecting group includes a portion of a branching group and linking group. In certain embodiments, a connecting group includes a branching group and portion of a linking group. In certain embodiments, a connecting group includes a portion of a branching group and portion of a linking group.

i. Certain Tethers

In certain embodiments, conjugate groups comprise one or more tethers covalently attached to the branching group. In certain embodiments, conjugate groups comprise one or more tethers covalently attached to the linking group. In certain embodiments, each tether is a linear aliphatic group comprising one or more groups selected from alkyl, ether, thioether, disulfide, amide and polyethylene glycol groups in any combination. In certain embodiments, each tether is a linear aliphatic group comprising one or more groups selected from alkyl, substituted alkyl, ether, thioether, disulfide, amide, phosphodiester and polyethylene glycol groups in any combination. In certain embodiments, each tether is a linear aliphatic group comprising one or more groups selected from alkyl, ether and amide groups in any combination. In certain embodiments, each tether is a linear aliphatic group comprising one or more groups selected from alkyl, substituted alkyl, phosphodiester, ether and amide groups in any combination. In certain embodiments, each tether is a linear aliphatic group comprising one or more groups selected from alkyl and phosphodiester in any combination. In certain embodiments, each tether comprises at least one phosphorus linking group or neutral linking group.

In certain embodiments, the tether includes one or more cleavable bond. In certain embodiments, the tether is attached to the branching group through either an amide or an ether group. In certain embodiments, the tether is attached to the branching group through a phosphodiester group. In certain embodiments, the tether is attached to the branching group through a phosphorus linking group or neutral linking group. In certain embodiments, the tether is attached to the branching group through an ether group. In certain embodiments, the tether is attached to the ligand through either an amide or an ether group. In certain embodiments, the tether is attached to the ligand through an ether group. In certain embodiments, the tether is attached to the ligand through either an amide or an ether group. In certain embodiments, the tether is attached to the ligand through an ether group.

In certain embodiments, each tether comprises from about 8 to about 20 atoms in chain length between the ligand and the branching group. In certain embodiments, each tether group comprises from about 10 to about 18 atoms in chain length between the ligand and the branching group. In certain embodiments, each tether group comprises about 13 atoms in chain length.

In certain embodiments, a tether has a structure selected from among:

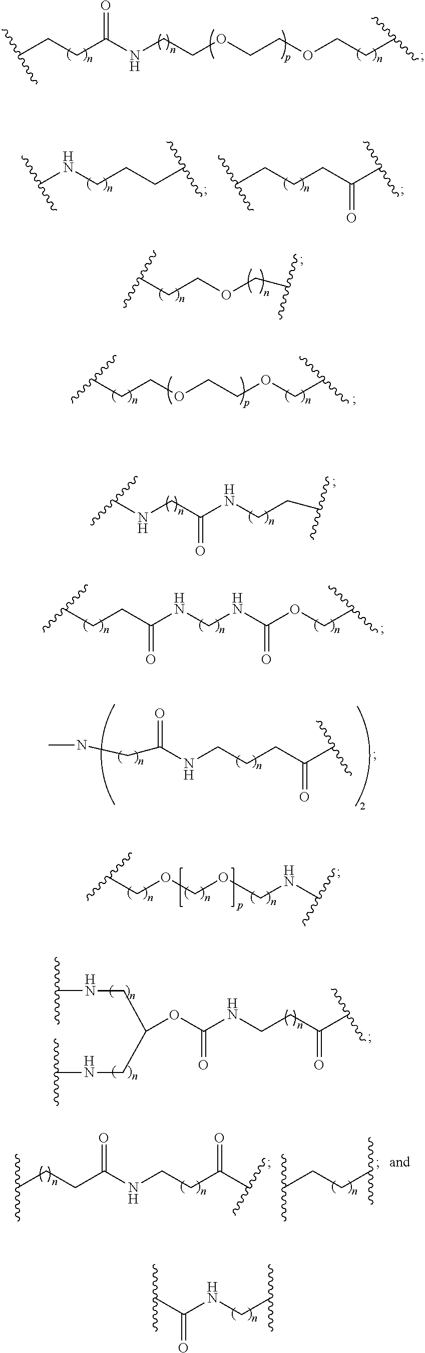

wherein each n is, independently, from 1 to 20; and
each p is from 1 to about 6.

In certain embodiments, a tether has a structure selected from among:

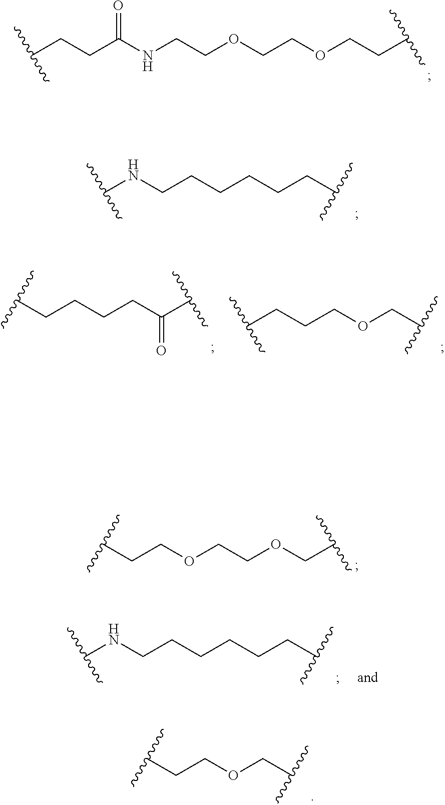

In certain embodiments, a tether has a structure selected from among:

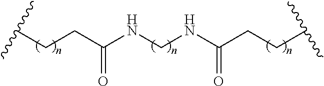

wherein each n is, independently, from 1 to 20.

In certain embodiments, a tether has a structure selected from among:

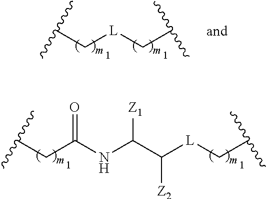

wherein L is either a phosphorus linking group or a neutral linking group;
Z₁ is C(═O)O—R₂;
Z₂ is H, C₁-C₆ alkyl or substituted C₁-C₆ alky;
R₂ is H, C₁-C₆ alkyl or substituted C₁-C₆ alky; and
   each m₁ is, independently, from 0 to 20 wherein at least one m₁ is greater than 0 for each tether.

In certain embodiments, a tether has a structure selected from among:

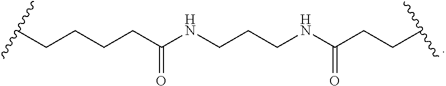 [SMILES: *CCCCC(=O)NCCCNC(=O)CC*]

In certain embodiments, a tether has a structure selected from among:

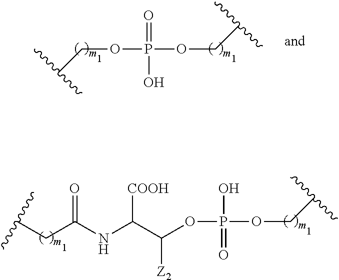

wherein Z₂ is H or CH₃; and
   each m₁ is, independently, from 0 to 20 wherein at least one m₁ is greater than 0 for each tether.

In certain embodiments, a tether has a structure selected from among:

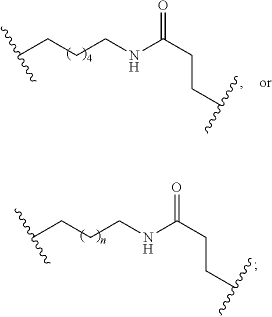

wherein each n is independently, 0, 1, 2, 3, 4, 5, 6, or 7.

In certain embodiments, a tether comprises a phosphorus linking group. In certain embodiments, a tether does not comprise any amide bonds. In certain embodiments, a tether comprises a phosphorus linking group and does not comprise any amide bonds.

ii. Certain Linking Groups

In certain embodiments, the conjugate groups comprise a linking group. In certain such embodiments, the linking group is covalently bound to the cleavable moiety. In certain such embodiments, the linking group is covalently bound to the antisense oligonucleotide. In certain embodiments, the linking group is covalently bound to a cell-targeting moiety. In certain embodiments, the linking group further comprises a covalent attachment to a solid support. In certain embodiments, the linking group further comprises a covalent attachment to a protein binding moiety. In certain embodiments, the linking group further comprises a covalent attachment to a solid support and further comprises a covalent attachment to a protein binding moiety. In certain embodiments, the linking group includes multiple positions for attachment of tethered ligands. In certain embodiments, the linking group includes multiple positions for attachment of tethered ligands and is not attached to a branching group. In certain embodiments, the linking group further comprises one or more cleavable bond. In certain embodiments, the conjugate group does not include a linking group.

In certain embodiments, the linking group includes at least a linear group comprising groups selected from alkyl, amide, disulfide, polyethylene glycol, ether, thioether (—S—) and hydroxylamino (—O—N(H)—) groups. In certain embodiments, the linear group comprises groups selected from alkyl, amide and ether groups. In certain embodiments, the linear group comprises groups selected from alkyl and ether groups. In certain embodiments, the linear group comprises at least one phosphorus linking group. In certain embodiments, the linear group comprises at least one phosphodiester group. In certain embodiments, the linear group includes at least one neutral linking group. In certain embodiments, the linear group is covalently attached to the cell-targeting moiety and the cleavable moiety. In certain embodiments, the linear group is covalently attached to the cell-targeting moiety and the antisense oligonucleotide. In certain embodiments, the linear group is covalently attached to the cell-targeting moiety, the cleavable moiety and a solid support. In certain embodiments, the linear group is covalently attached to the cell-targeting moiety, the cleavable moiety, a solid support and a protein binding moiety. In certain embodiments, the linear group includes one or more cleavable bond.

In certain embodiments, the linking group includes the linear group covalently attached to a scaffold group. In certain embodiments, the scaffold includes a branched aliphatic group comprising groups selected from alkyl, amide, disulfide, polyethylene glycol, ether, thioether and hydroxylamino groups. In certain embodiments, the scaffold includes a branched aliphatic group comprising groups selected from alkyl, amide and ether groups. In certain embodiments, the scaffold includes at least one mono or polycyclic ring system. In certain embodiments, the scaffold includes at least two mono or polycyclic ring systems. In certain embodiments, the linear group is covalently attached to the scaffold group and the scaffold group is covalently attached to the cleavable moiety and the linking group. In certain embodiments, the linear group is covalently attached to the scaffold group and the scaffold group is covalently attached to the cleavable moiety, the linking group and a solid support. In certain embodiments, the linear group is covalently attached to the scaffold group and the scaffold group is covalently attached to the cleavable moiety, the linking group and a protein binding moiety. In certain embodiments, the linear group is covalently attached to the scaffold group and the scaffold group is covalently attached to the cleavable moiety, the linking group, a protein binding moiety and a solid support. In certain embodiments, the scaffold group includes one or more cleavable bond.

In certain embodiments, the linking group includes a protein binding moiety. In certain embodiments, the protein binding moiety is a lipid such as for example including but not limited to cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, 03-(oleoyl)lithocholic acid, 03-(oleoyl)cholenic acid, dimethoxytrityl, or phenoxazine), a vitamin (e.g., folate, vitamin A, vitamin E, biotin, pyridoxal), a peptide, a carbohydrate (e.g., monosaccharide, disaccharide, trisaccharide, tetrasaccharide, oligosaccharide, polysaccharide), an endosomolytic component, a steroid (e.g., uvaol, hecigenin, diosgenin), a terpene (e.g., triterpene, e.g., sarsasapogenin, friedelin, epifriedelanol derivatized lithocholic acid), or a cationic lipid. In certain embodiments, the protein binding moiety is a C₁₆ to C₂₂ long chain saturated or unsaturated fatty acid, cholesterol, cholic acid, vitamin E, adamantane or 1-pentafluoropropyl.

In certain embodiments, a linking group has a structure selected from among:

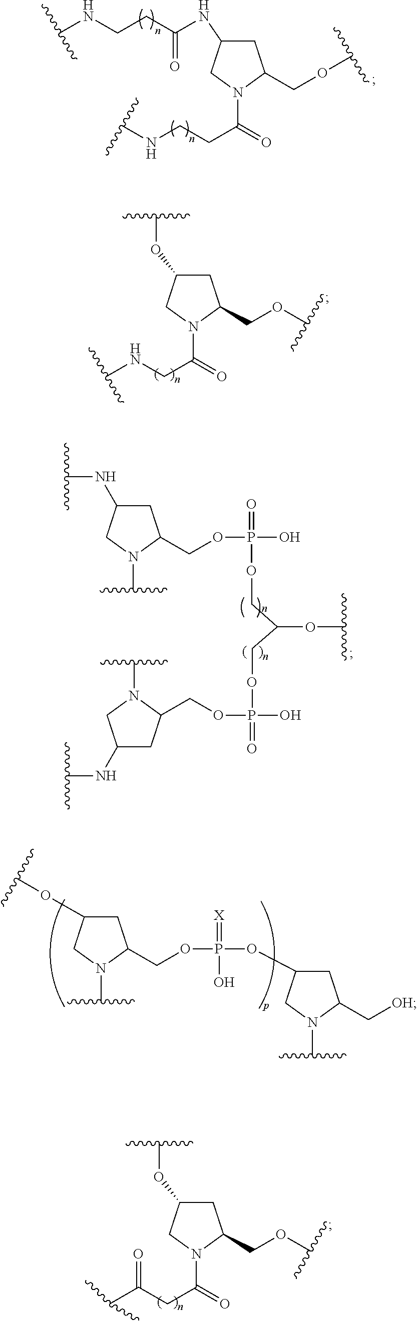

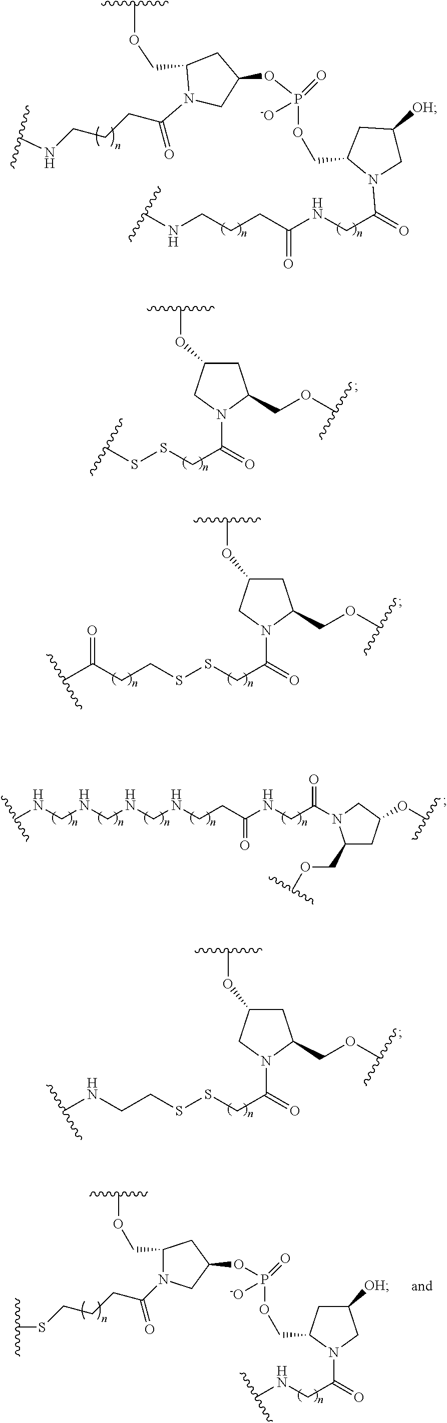

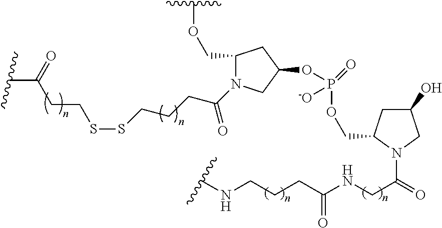

wherein each n is, independently, from 1 to 20; and p is from 1 to 6.

In certain embodiments, a linking group has a structure selected from among:

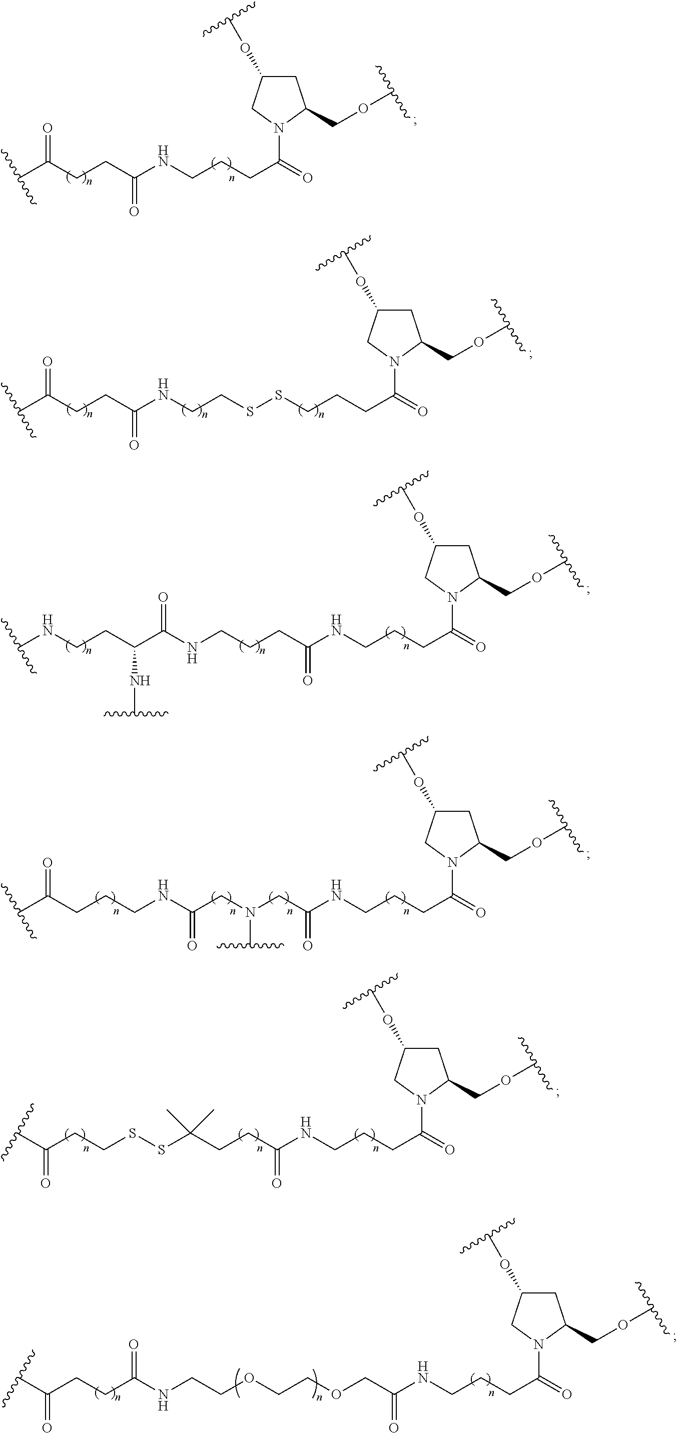

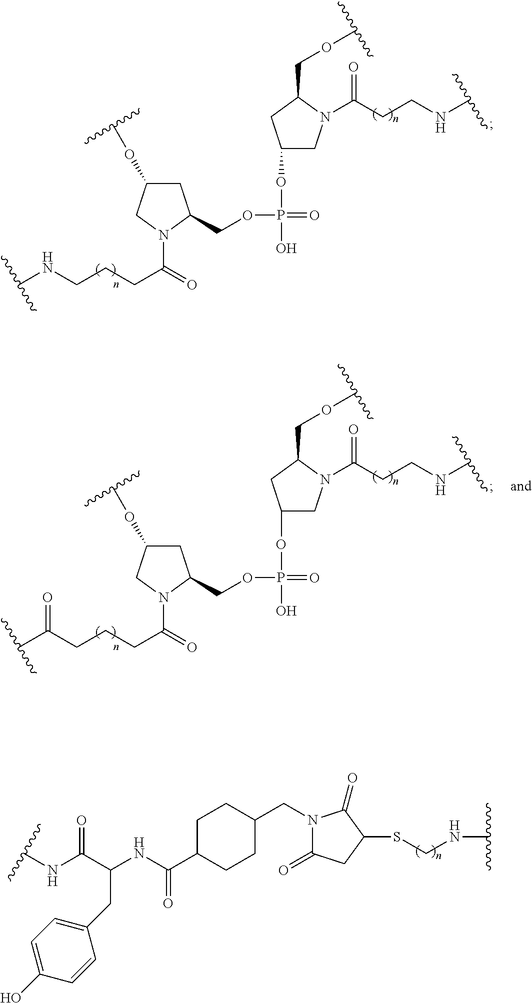

wherein each n is, independently, from 1 to 20.

In certain embodiments, a linking group has a structure selected from among:

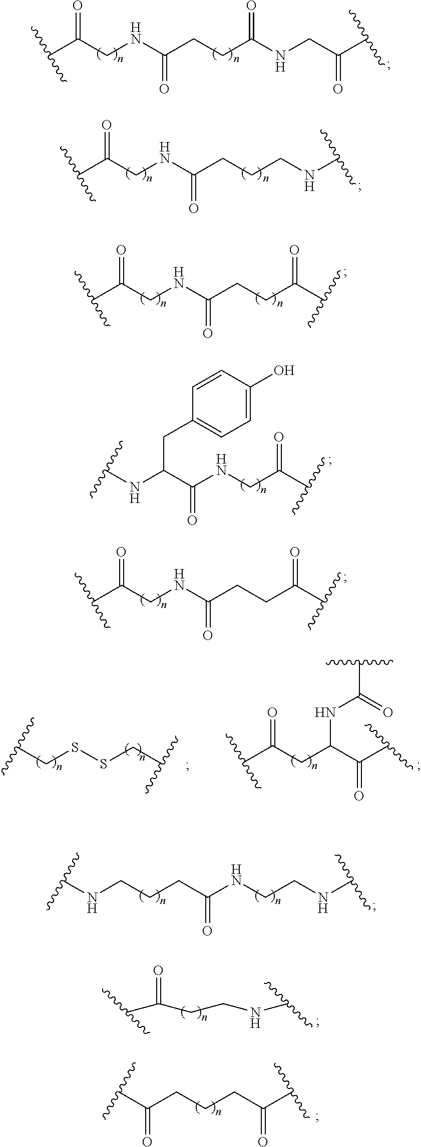

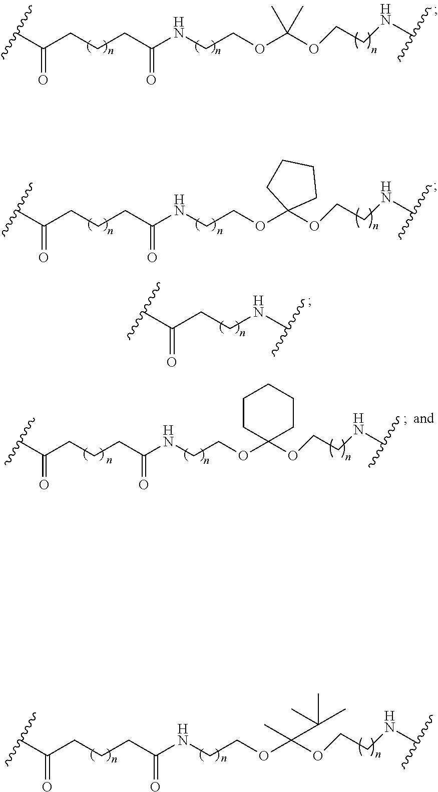

wherein n is from 1 to 20.

In certain embodiments, a linking group has a structure selected from among:

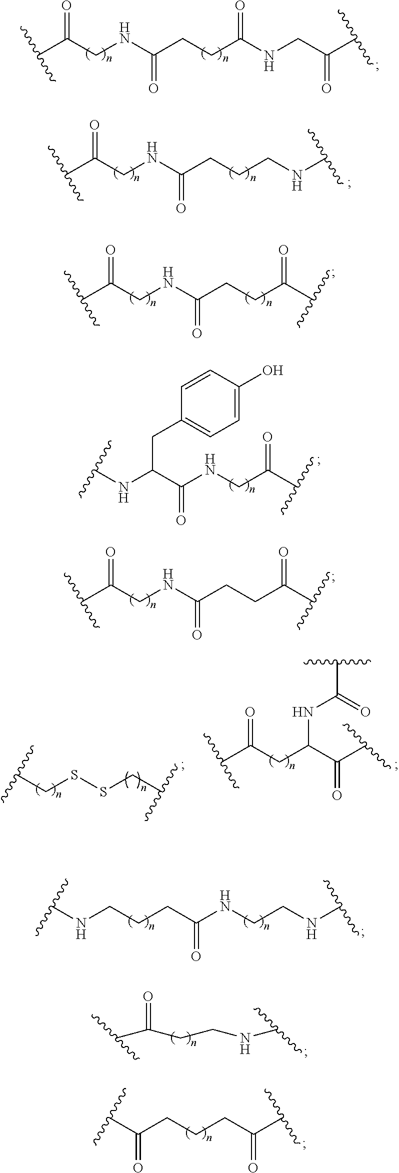

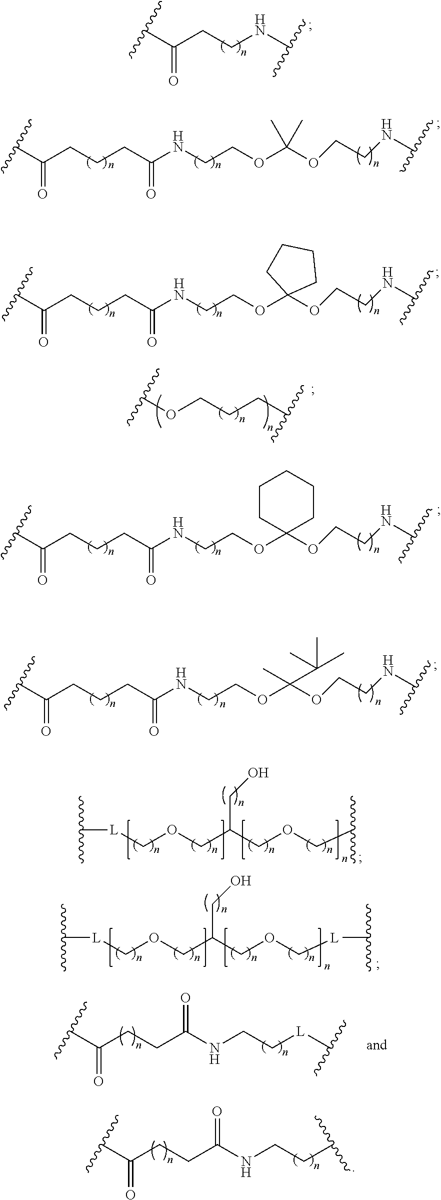

wherein each L is, independently, a phosphorus linking group or a neutral linking group; and each n is, independently, from 1 to 20.

In certain embodiments, a linking group has a structure selected from among:

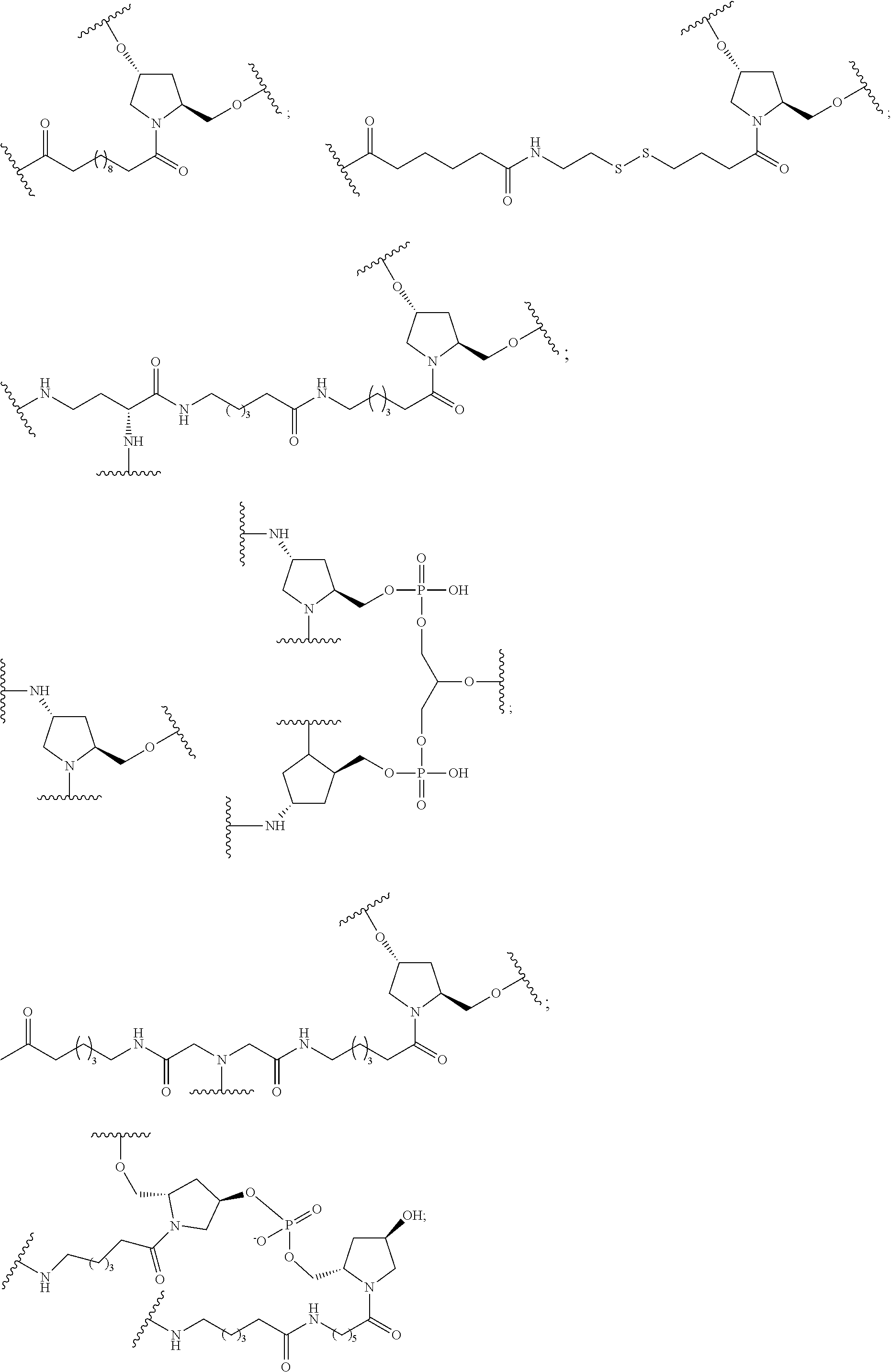

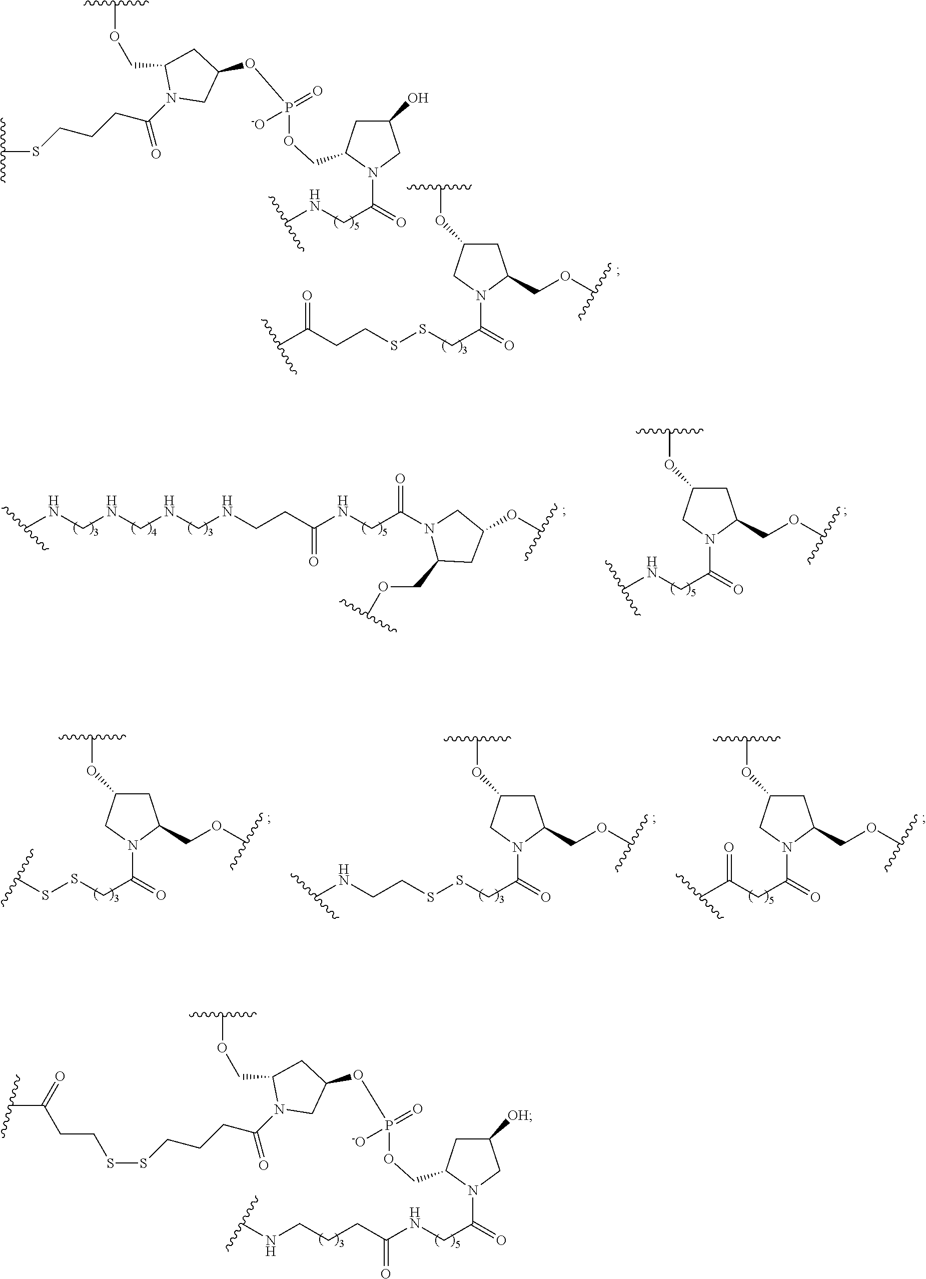

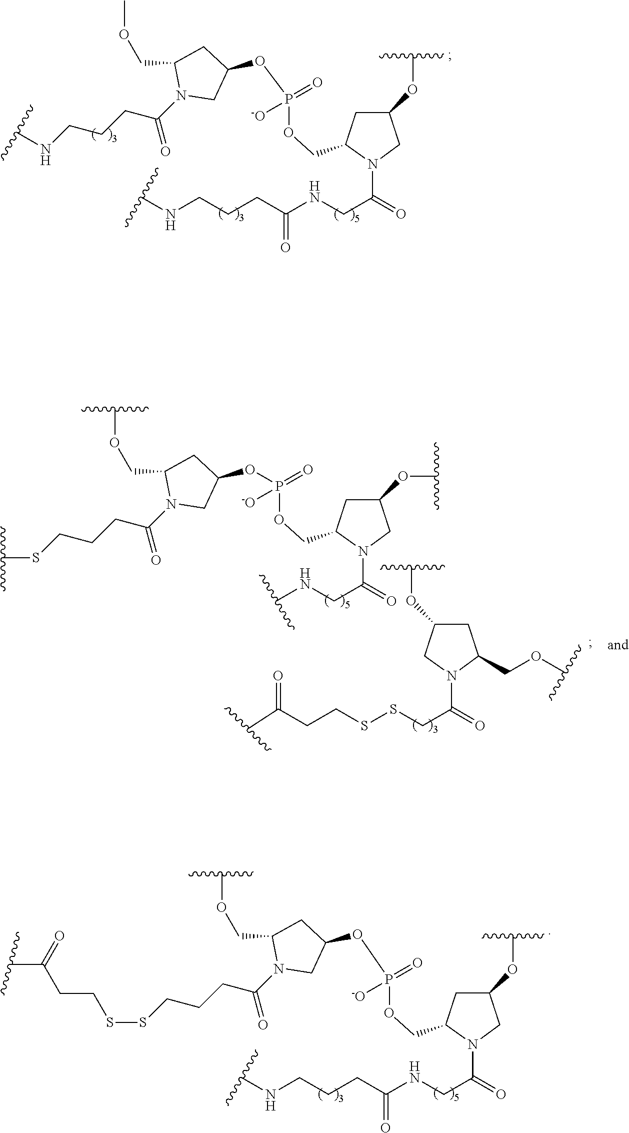

In certain embodiments, a linking group has a structure selected from among:

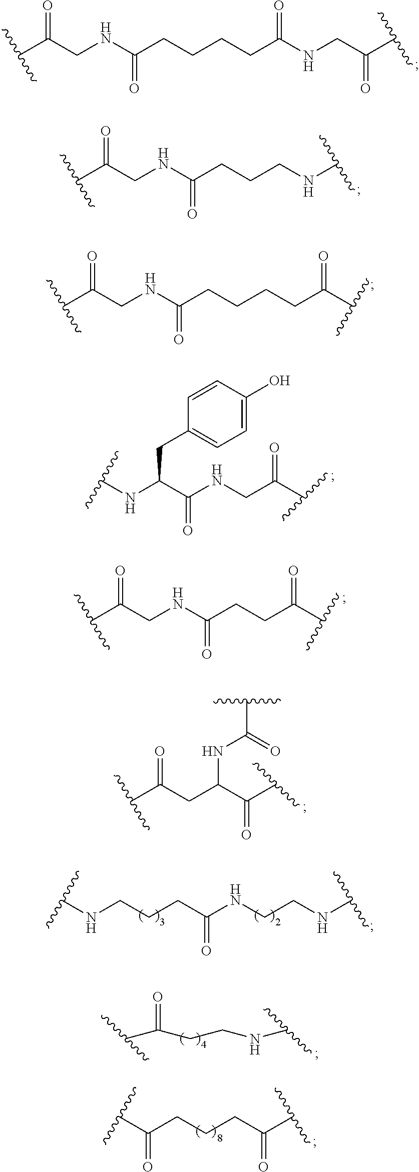

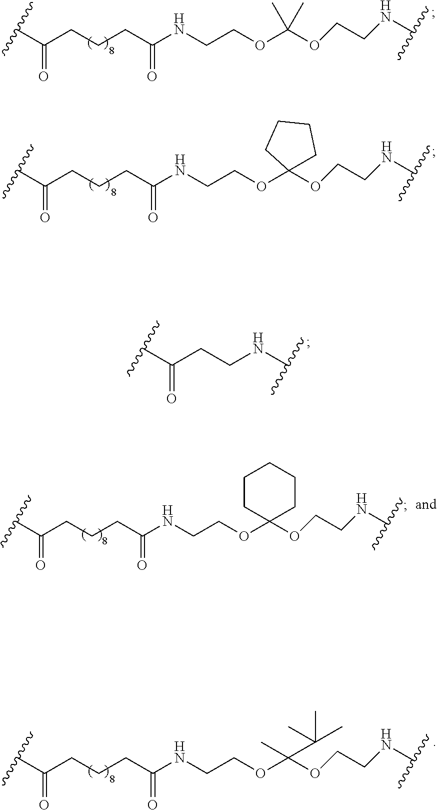

In certain embodiments, a linking group has a structure selected from among:

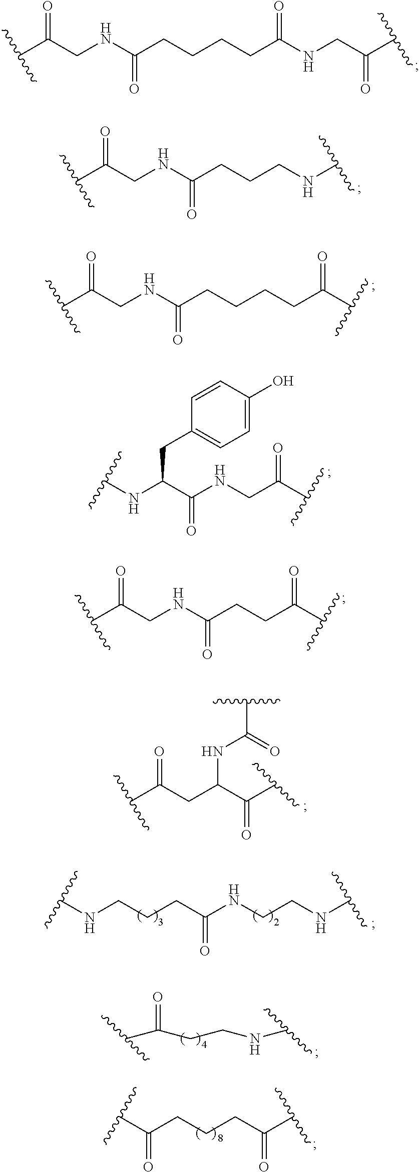

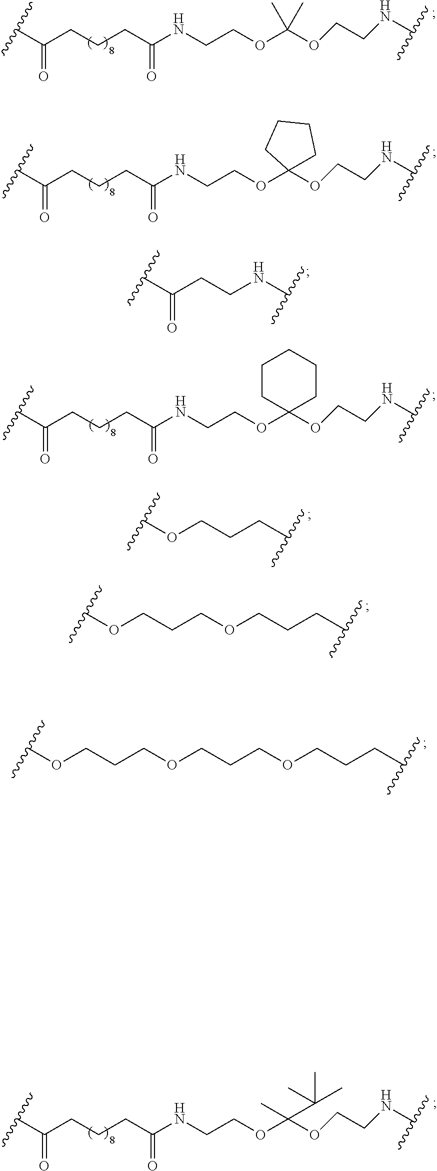

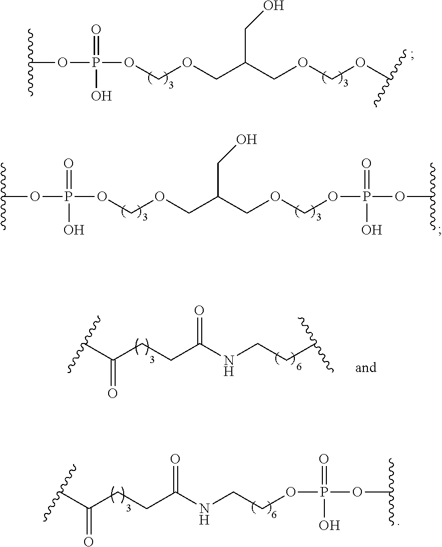

In certain embodiments, a linking group has a structure selected from among:

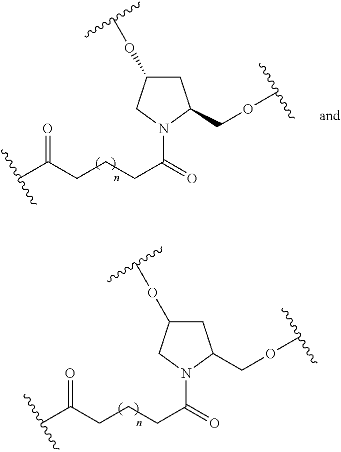

wherein n is from 1 to 20.

In certain embodiments, a linking group has a structure selected from among:

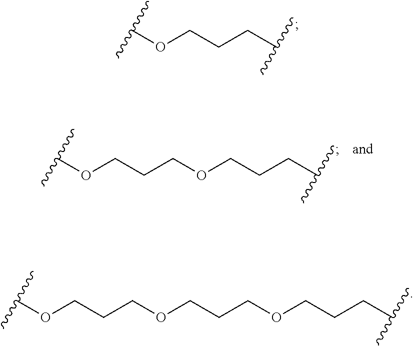 [SMILES: *CCCO* ; *CCCOCCCO* ; *CCCOCCCOCCCO*]

In certain embodiments, a linking group has a structure selected from among:

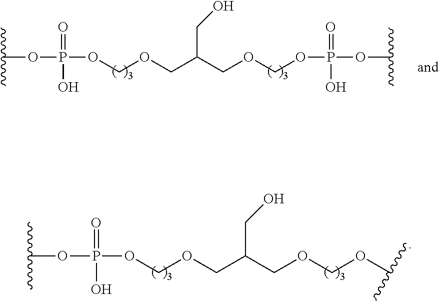 [SMILES: *OP(=O)(O)OCOCC(CO)COCOP(=O)(O)O* ; *OCOCC(CO)COCOP(=O)(O)O*]

In certain embodiments, a linking group has a structure selected from among:

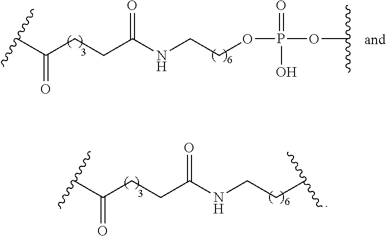 [SMILES: *OP(=O)(O)OCCNC(=O)CCC(*)=O ; *CCNC(=O)CCC(*)=O]

In certain embodiments, the conjugate linking group has the structure:

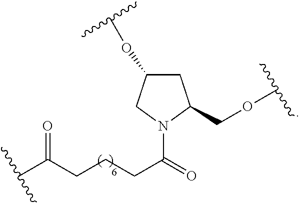 [SMILES: *OC[C@@H]1C[C@@H](O*)CN1C(=O)CCCC(*)=O]

In certain embodiments, the conjugate linking group has the structure:

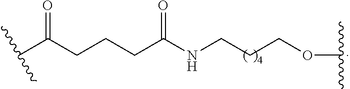 [SMILES: *OCCCNC(=O)CCCC(*)=O]

In certain embodiments, a linking group has a structure selected from among:

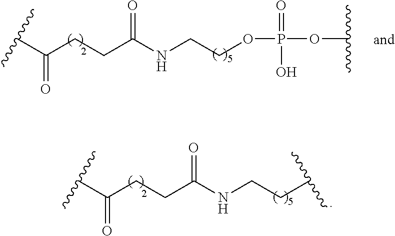 [SMILES: *OP(=O)(O)OCCNC(=O)CCC(*)=O ; *CCNC(=O)CCC(*)=O]

In certain embodiments, a linking group has a structure selected from among:

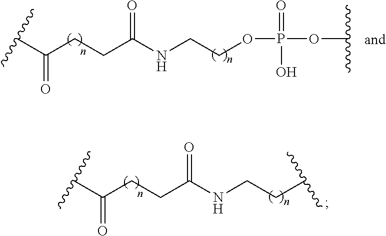

wherein each n is independently, 0, 1, 2, 3, 4, 5, 6, or 7.
c. Certain Branching Groups

In certain embodiments, the conjugate groups comprise a targeting moiety comprising a branching group and at least two tethered ligands. In certain embodiments, the branching group attaches the conjugate linker. In certain embodiments, the branching group attaches the cleavable moiety. In certain embodiments, the branching group attaches the antisense oligonucleotide. In certain embodiments, the branching group is covalently attached to the linker and each of the tethered ligands. In certain embodiments, the branching group comprises a branched aliphatic group comprising groups selected from alkyl, amide, disulfide, polyethylene glycol, ether, thioether and hydroxylamino groups. In certain embodiments, the branching group comprises groups selected from alkyl, amide and ether groups. In certain embodiments, the branching group comprises groups selected from alkyl and ether groups. In certain embodiments, the branching group comprises a mono or polycyclic ring system. In certain embodiments, the branching group comprises one or more cleavable bond. In certain embodiments, the conjugate group does not include a branching group.

In certain embodiments, a branching group has a structure selected from among:

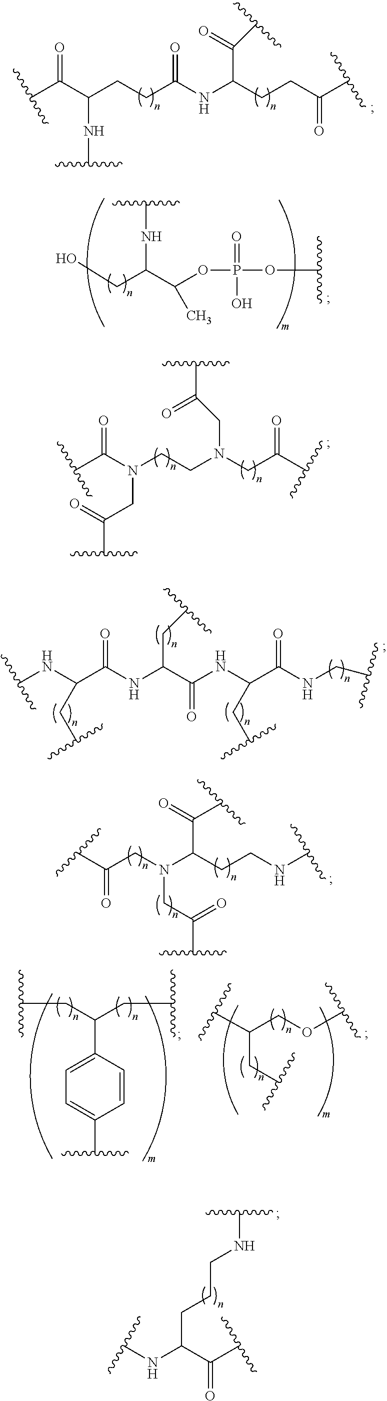

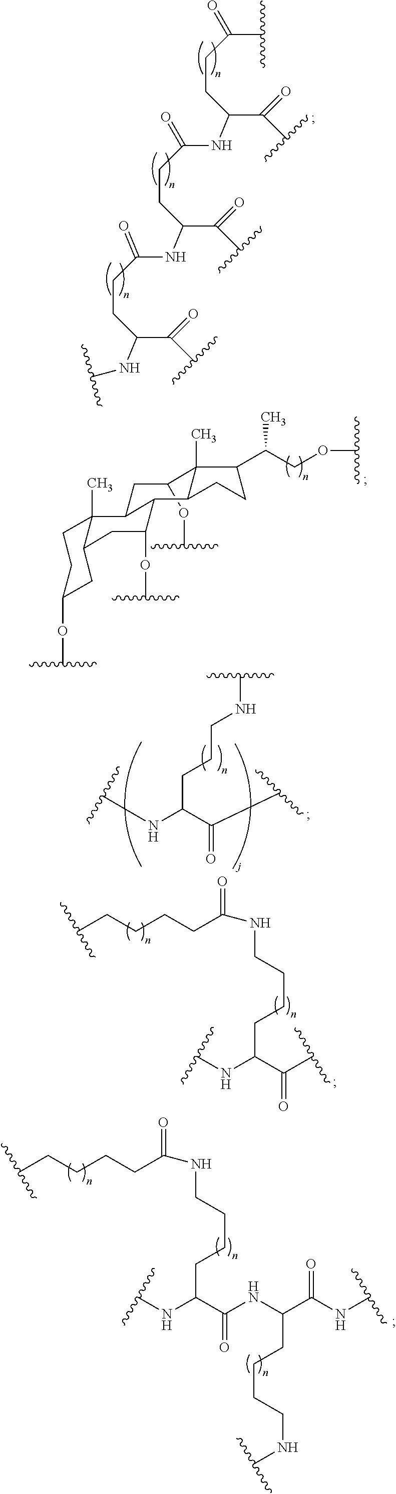

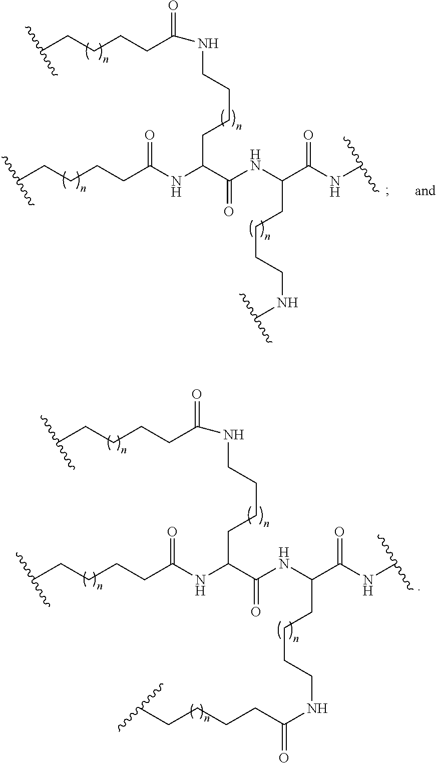

wherein each n is, independently, from 1 to 20;
J is from 1 to 3; and
m is from 2 to 6.

In certain embodiments, a branching group has a structure selected from among:

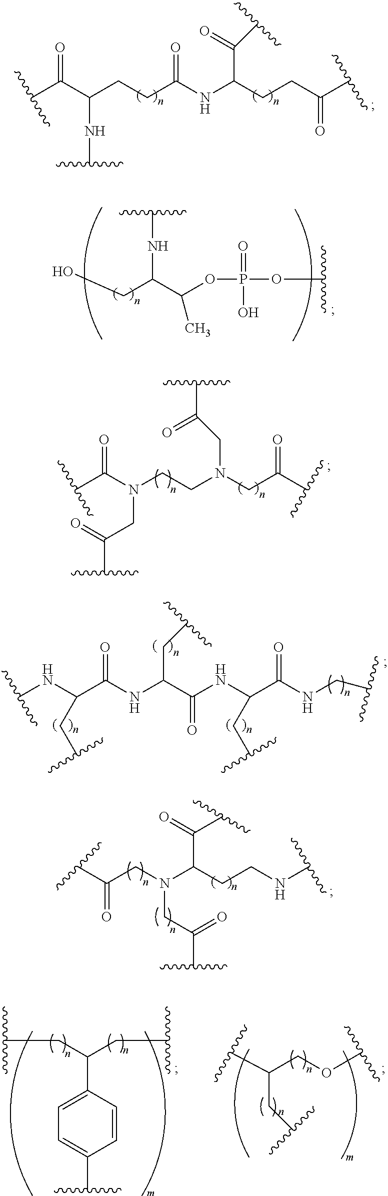

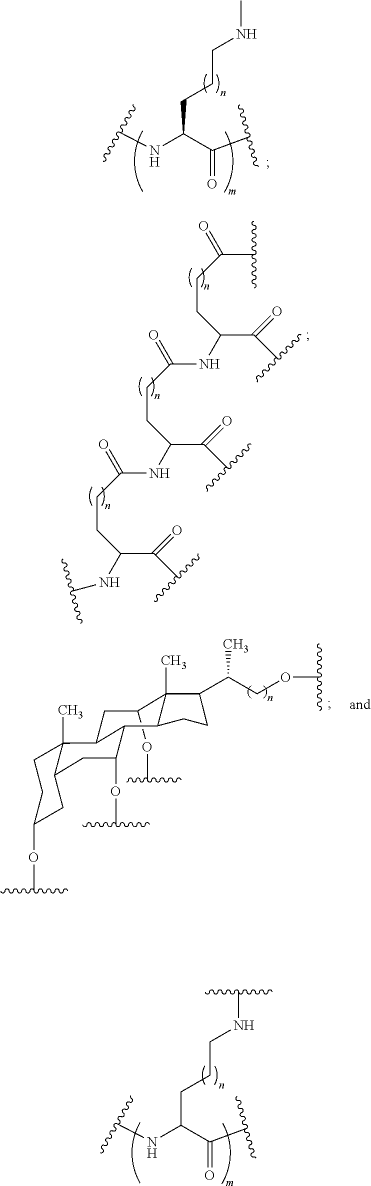

wherein each n is, independently, from 1 to 20; and
m is from 2 to 6.

In certain embodiments, a branching group has a structure selected from among:

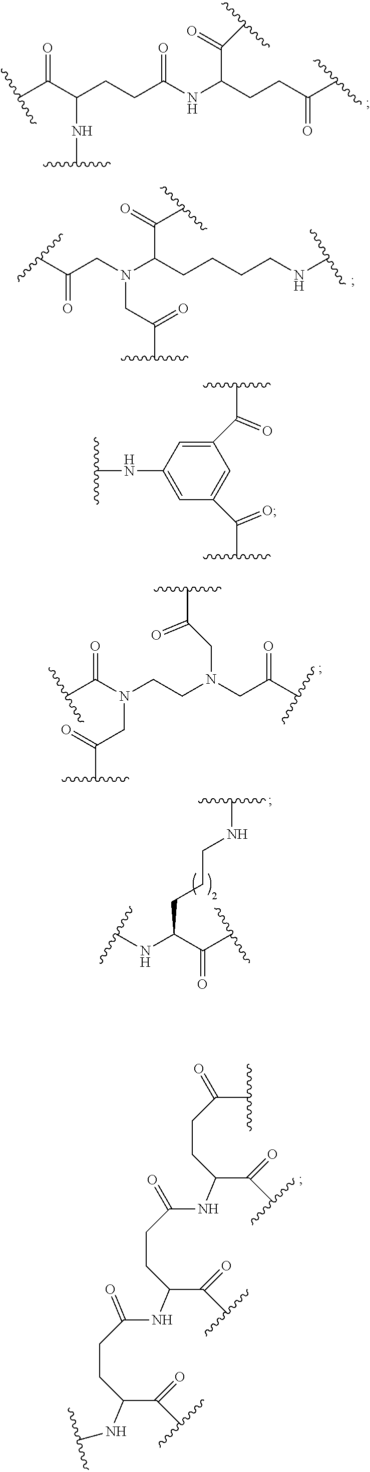

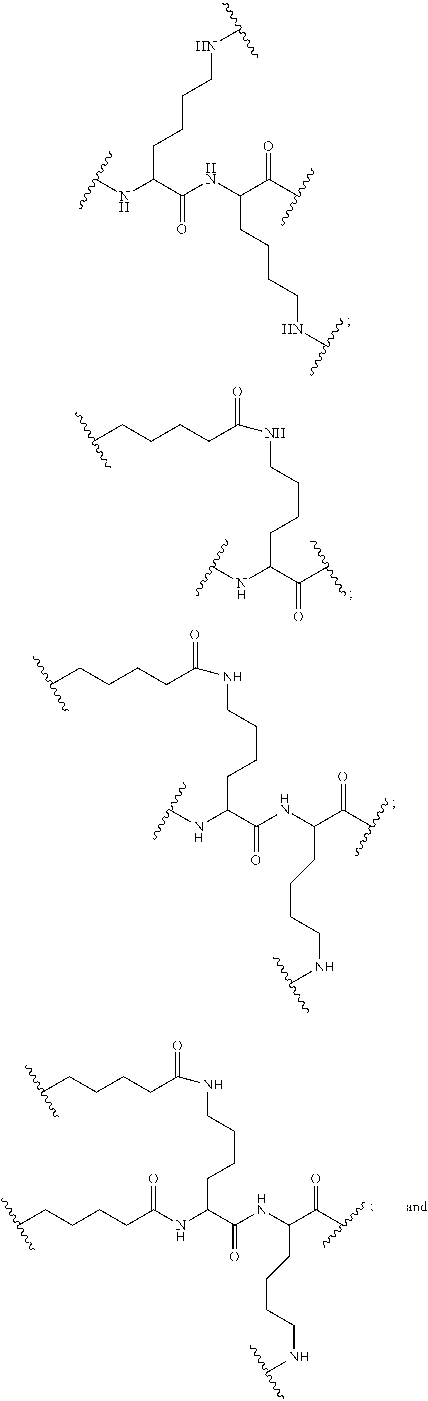 [SMILES: *NCCCCC(NC(=O)C(CCCCN*)N*)C(*)=O ; *CCCCC(=O)NCCCCC(N*)C(*)=O ; *CCCCC(=O)NCCCCC(N*)C(=O)NC(CCCCN*)C(*)=O ; *CCCCC(=O)NCCCCC(NC(=O)CCCC*)C(=O)NC(CCCCN*)C(*)=O]

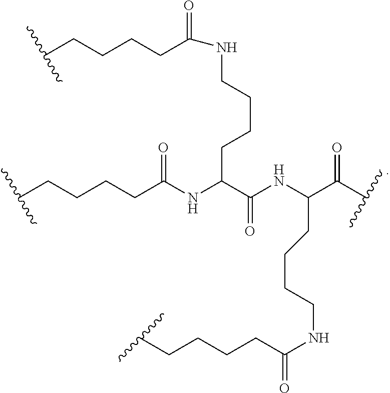 [SMILES: *CCCCC(=O)NCCCCC(NC(=O)C(CCCCNC(=O)CCCC*)NC(=O)CCCC*)C(*)=O]

In certain embodiments, a branching group has a structure selected from among:

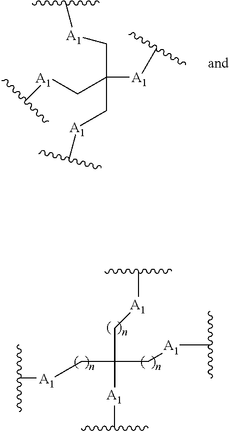

wherein each A₁ is independently, O, S, C═O or NH; and
each n is, independently, from 1 to 20.

In certain embodiments, a branching group has a structure selected from among:

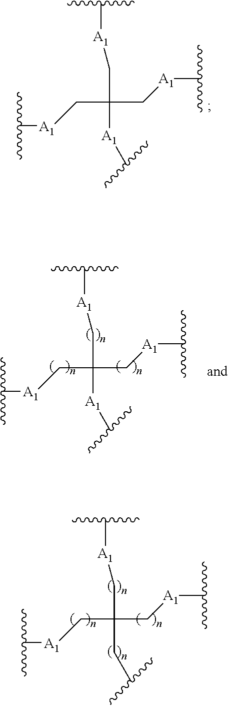

wherein each A₁ is independently, O, S, C═O or NH; and
each n is, independently, from 1 to 20.

In certain embodiments, a branching group has a structure selected from among:

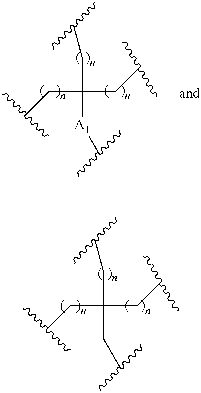

wherein A₁ is O, S, C═O or NH; and
each n is, independently, from 1 to 20.

In certain embodiments, a branching group has a structure selected from among:

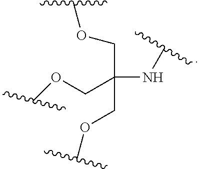 [SMILES: *NC(CO*)(CO*)CO*]

In certain embodiments, a branching group has a structure selected from among:

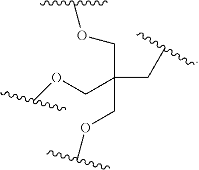 [SMILES: *CC(CO*)(CO*)CO*]

In certain embodiments, a branching group has a structure selected from among:

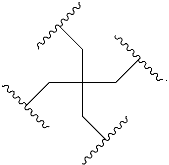 [SMILES: *CC(C*)(C*)C*]

d. Certain Cleavable Moieties

In certain embodiments, a cleavable moiety is a cleavable bond. In certain embodiments, a cleavable moiety comprises a cleavable bond. In certain embodiments, the conjugate group comprises a cleavable moiety. In certain such embodiments, the cleavable moiety attaches to the antisense oligonucleotide. In certain such embodiments, the cleavable moiety attaches directly to the cell-targeting moiety. In certain such embodiments, the cleavable moiety attaches to the conjugate linker. In certain embodiments, the cleavable moiety comprises a phosphate or phosphodiester. In certain embodiments, the cleavable moiety is a cleavable nucleoside or nucleoside analog. In certain embodiments, the nucleoside or nucleoside analog comprises an optionally protected heterocyclic base selected from a purine, substituted purine, pyrimidine or substituted pyrimidine. In certain embodiments, the cleavable moiety is a nucleoside comprising an optionally protected heterocyclic base selected from uracil, thymine, cytosine, 4-N-benzoylcytosine, 5-methylcytosine, 4-N-benzoyl-5-methylcytosine, adenine, 6-N-benzoyladenine, guanine and 2-N-isobutyrylguanine. In certain embodiments, the cleavable moiety is 2′-deoxy nucleoside that is attached to the 3′ position of the antisense oligonucleotide by a phosphodiester linkage and is attached to the linker by a phosphodiester or phosphorothioate linkage. In certain embodiments, the cleavable moiety is 2′-deoxy adenosine that is attached to the 3′ position of the antisense oligonucleotide by a phosphodiester linkage and is attached to the linker by a phosphodiester or phosphorothioate linkage. In certain embodiments, the cleavable moiety is 2′-deoxy adenosine that is attached to the 3′ position of the antisense oligonucleotide by a phosphodiester linkage and is attached to the linker by a phosphodiester linkage.

In certain embodiments, the cleavable moiety is attached to the 3′ position of the antisense oligonucleotide. In certain embodiments, the cleavable moiety is attached to the 5′ position of the antisense oligonucleotide. In certain embodiments, the cleavable moiety is attached to a 2′ position of the antisense oligonucleotide. In certain embodiments, the cleavable moiety is attached to the antisense oligonucleotide by a phosphodiester linkage. In certain embodiments, the cleavable moiety is attached to the linker by either a phosphodiester or a phosphorothioate linkage. In certain embodiments, the cleavable moiety is attached to the linker by a phosphodiester linkage. In certain embodiments, the conjugate group does not include a cleavable moiety.

In certain embodiments, the cleavable moiety is cleaved after the complex has been administered to an animal only after being internalized by a targeted cell. Inside the cell the cleavable moiety is cleaved thereby releasing the active antisense oligonucleotide. While not wanting to be bound by theory it is believed that the cleavable moiety is cleaved by one or more nucleases within the cell. In certain embodiments, the one or more nucleases cleave the phosphodiester linkage between the cleavable moiety and the linker. In certain embodiments, the cleavable moiety has a structure selected from among the following:

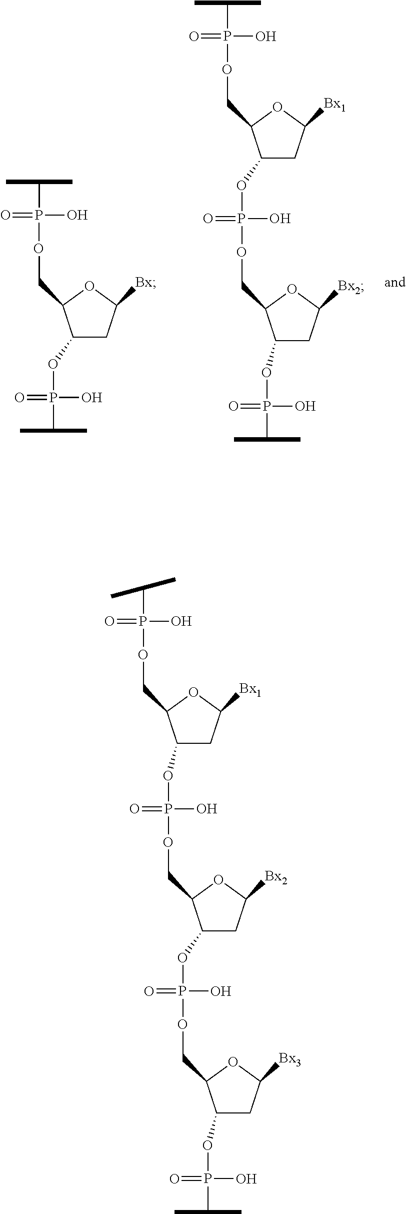

wherein each of Bx, Bx₁, Bx₂, and Bx₃ is independently a heterocyclic base moiety. In certain embodiments, the cleavable moiety has a structure selected from among the following:

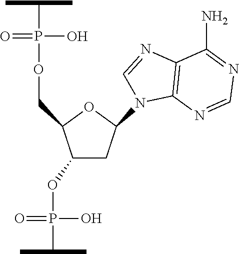

e. Certain Cell-Targeting Moieties

In certain embodiments, conjugate groups comprise cell-targeting moieties. Certain such cell-targeting moieties increase cellular uptake of antisense compounds. In certain embodiments, cell-targeting moieties comprise a branching group, one or more tether, and one or more ligand. In certain embodiments, cell-targeting moieties comprise a branching group, one or more tether, one or more ligand and one or more cleavable bond. In certain embodiments, cell-targeting moieties comprise a portion of a connecting group. In certain embodiments, cell-targeting moieties comprise a portion of a connecting group and one or more ligands. In certain embodiments, cell-targeting moieties comprise a portion of a connecting group and one ligand. In certain embodiments, cell-targeting moieties comprise a portion of a connecting group and two ligands. In certain embodiments, cell-targeting moieties comprise a portion of a connecting group three ligands.

f. Certain Ligands

In certain embodiments, the present disclosure provides ligands wherein each ligand is covalently attached to a tether. In certain embodiments, each ligand is selected to have an affinity for at least one type of receptor on a target cell. In certain embodiments, ligands are selected that have an affinity for at least one type of receptor on the surface of a mammalian liver cell. In certain embodiments, ligands are selected that have an affinity for the hepatic asialoglycoprotein receptor (ASGP-R). In certain embodiments, each ligand is a carbohydrate. In certain embodiments, each ligand is, independently selected from galactose, N-acetyl galactoseamine, mannose, glucose, glucosamone and fucose. In certain embodiments, each ligand is N-acetyl galactoseamine (GalNAc). In certain embodiments, the targeting moiety comprises 2 to 6 ligands. In certain embodiments, the targeting moiety comprises 3 ligands. In certain embodiments, the targeting moiety comprises 3 N-acetyl galactoseamine ligands.

In certain embodiments, the ligand is a carbohydrate, carbohydrate derivative, modified carbohydrate, multivalent carbohydrate cluster, polysaccharide, modified polysaccharide, or polysaccharide derivative. In certain embodiments, the ligand is an amino sugar or a thio sugar. For example, amino sugars may be selected from any number of compounds known in the art, for example glucosamine, sialic acid, α-D-galactosamine, N-Acetylgalactosamine, 2-acetamido-2-deoxy-D-galactopyranose (GalNAc), 2-Amino-3-O—[(R)-1-carboxyethyl]-2-deoxy-β-D-glucopyranose (β-muramic acid), 2-Deoxy-2-methylamino-L-glucopyranose, 4,6-Dideoxy-4-formamido-2,3-di-O-methyl-D-mannopyranose, 2-Deoxy-2-sulfoamino-D-glucopyranose and N-sulfo-D-glucosamine, and N-Glycoloyl-α-neuraminic acid. For example, thio sugars may be selected from the group consisting of 5-Thio-β-D-glucopyranose, Methyl 2,3,4-tri-O-acetyl-1-thio-6-O-trityl-α-D-glucopyranoside, 4-Thio-β-D-galactopyranose, and ethyl 3,4,6,7-tetra-O-acetyl-2-deoxy-1,5-dithio-α-D-gluco-heptopyranoside.

In certain embodiments, “GalNac” or “Gal-NAc” refers to 2-(Acetylamino)-2-deoxy-D-galactopyranose, commonly referred to in the literature as N-acetyl galactosamine. In certain embodiments, “N-acetyl galactosamine” refers to 2-(Acetylamino)-2-deoxy-D-galactopyranose. In certain embodiments, “GalNac” or “Gal-NAc” refers to 2-(Acetylamino)-2-deoxy-D-galactopyranose. In certain embodiments, “GalNac” or “Gal-NAc” refers to 2-(Acetylamino)-2-deoxy-D-galactopyranose, which includes both the 3-form: 2-(Acetylamino)-2-deoxy-β-D-galactopyranose and α-form: 2-(Acetylamino)-2-deoxy-D-galactopyranose. In certain embodiments, both the β-form: 2-(Acetylamino)-2-deoxy-β-D-galactopyranose and α-form: 2-(Acetylamino)-2-deoxy-D-galactopyranose may be used interchangeably. Accordingly, in structures in which one form is depicted, these structures are intended to include the other form as well. For example, where the structure for an α-form: 2-(Acetylamino)-2-deoxy-D-galactopyranose is shown, this structure is intended to include the other form as well. In certain embodiments, In certain preferred embodiments, the β-form 2-(Acetylamino)-2-deoxy-D-galactopyranose is the preferred embodiment.

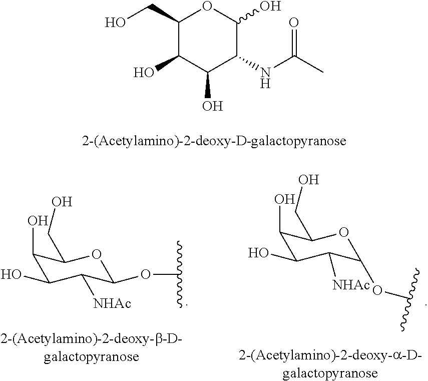 [SMILES: CC(=O)N[C@H]1C(O)O[C@H](CO)[C@H](O)[C@@H]1O ; *OC1OC(CO)C(O)C(O)C1NC(C)=O ; *OC1OC(CO)C(O)C(O)C1NC(C)=O]

In certain embodiments a compound comprises an oligomeric compound and a conjugate group, wherein the conjugate group comprises a moiety having Formula I:

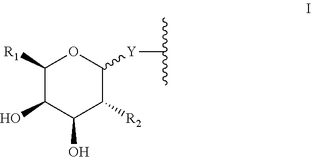

wherein:
R₁ is selected from Q₁, CH₂Q₁, CH₂OH, CH₂NJ₁J₂, CH₂N₃ and CH₂SJ₃;
Q₁ is selected from aryl, substituted aryl, heterocyclic, substituted heterocyclic, heteroaryl and substituted heteroaryl;
R₂ is selected from N₃, CN, halogen, N(H)C(═O)-Q₂, substituted thiol, aryl, substituted aryl, heterocyclic, substituted heterocyclic, heteroaryl and substituted heteroaryl;
Q₂ is selected from H, C₁-C₆ alkyl, substituted C₁-C₆ alkyl, C₁-C₆ alkoxy, substituted C₁-C₆ alkoxy, aryl, substituted aryl, heterocyclic, substituted heterocyclic, heteroaryl and substituted heteroaryl;
Y is selected from O, S, CJ₄J₅, NJ₆ and N(J₆)C(═O);
J₁, J₂, J₃, J₄, J₅, and J₆ are each, independently, H or a substituent group;
each substituent group is, independently, mono or poly substituted with optionally protected substituent groups independently selected from halogen, C₁-C₆ alkyl, C₁-C₆ alkoxy, aryl, heterocyclic and heteroaryl wherein each substituent group can include a connecting group comprising a linear alkyl group optionally including one or more groups independently selected from O, S, NH and C(═O), and wherein each substituent group may be further substituted with one or more groups independently selected from C₁-C₆ alkyl, halogen or C₁-C₆ alkoxy wherein each cyclic group is mono or polycyclic; and
when Y is O and R₁ is OH then R₂ is other than OH and N(H)C(═O)CH₃.
   g. Certain Conjugates

In certain embodiments, conjugate groups comprise the structural features above. In certain such embodiments, conjugate groups have the following structure:

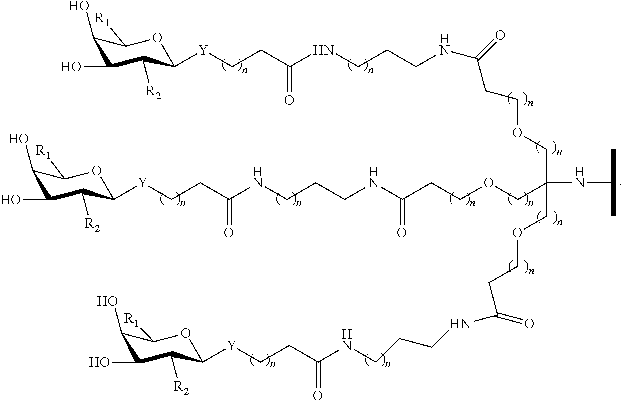

wherein each n is, independently, from 1 to 20.

In certain such embodiments, conjugate groups have the following structure:

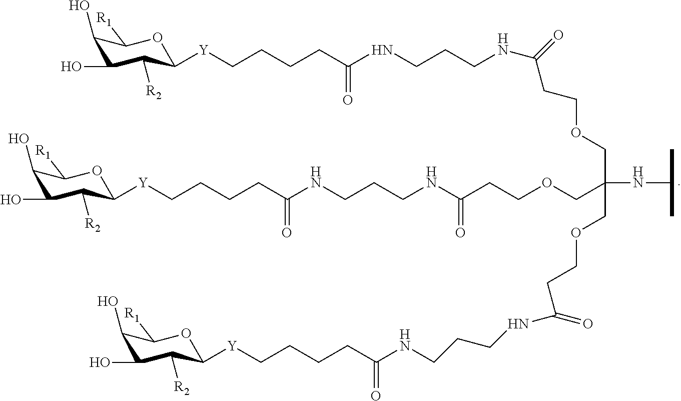

In certain such embodiments, conjugate groups have the following structure:

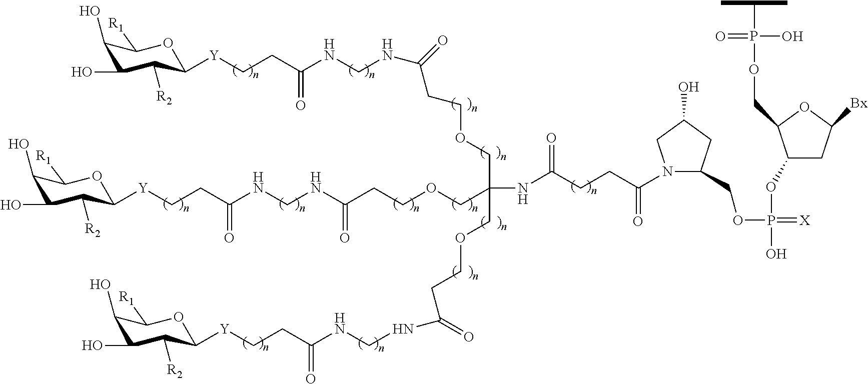

wherein each n is, independently, from 1 to 20;
Z is H or a linked solid support;
Q is an antisense compound;
X is O or S; and
Bx is a heterocyclic base moiety.

In certain such embodiments, conjugate groups have the following structure:

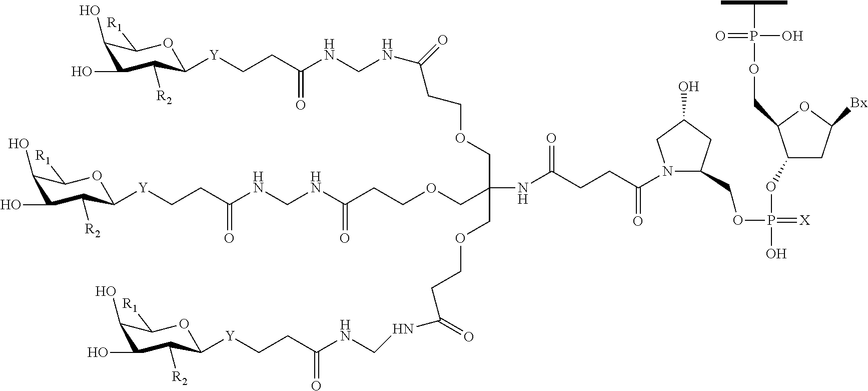

In certain such embodiments, conjugate groups have the following structure:

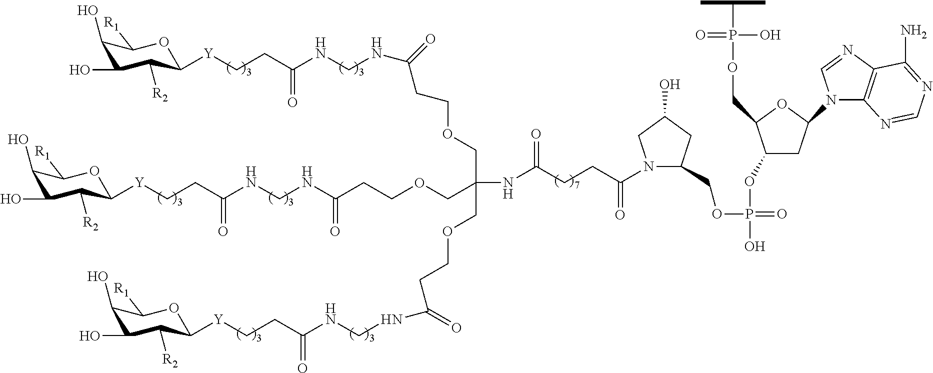

In certain such embodiments, conjugate groups have the following structure:

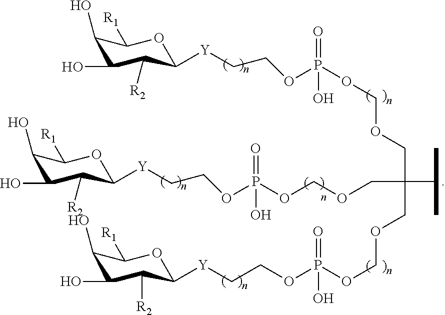

In certain such embodiments, conjugate groups have the following structure:

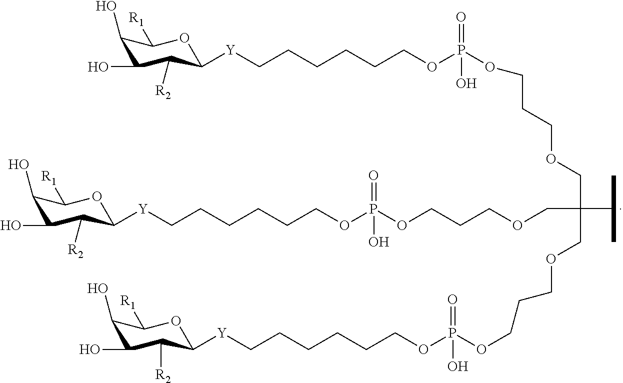

In certain such embodiments, conjugate groups have the following structure:

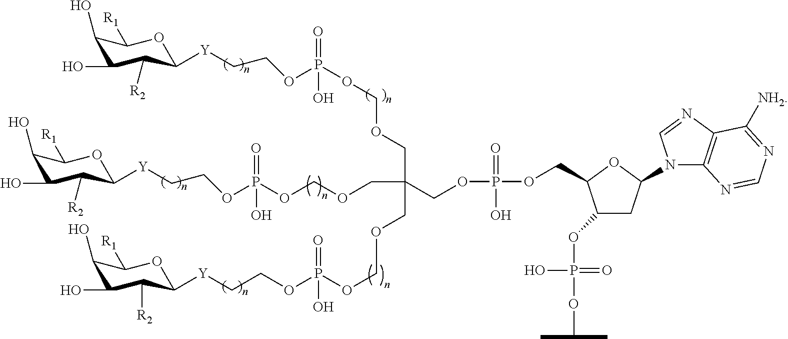

In certain such embodiments, conjugate groups have the following structure:

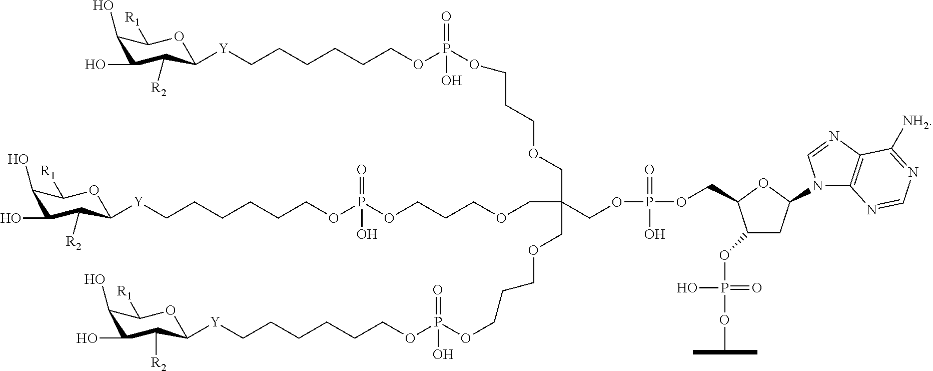

In certain such embodiments, conjugate groups have the following structure.

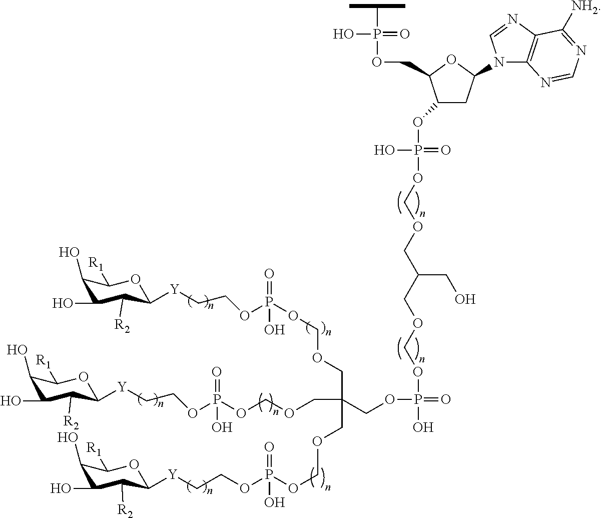

In certain such embodiments, conjugate groups have the following structure:

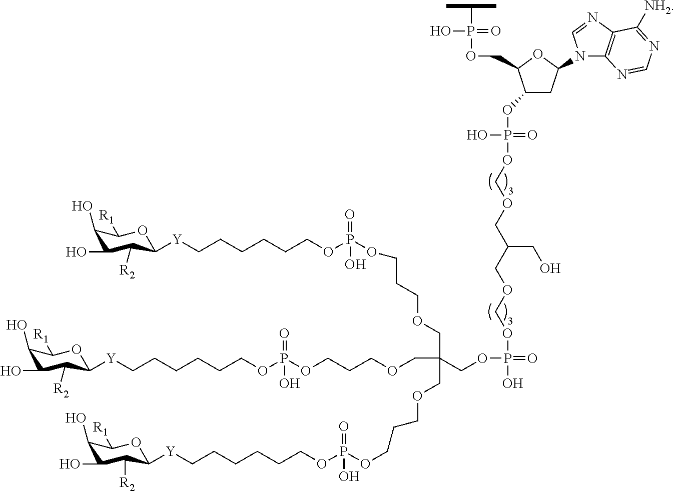

In certain embodiments, conjugates do not comprise a pyrrolidine.

In certain such embodiments, conjugate groups have the following structure:

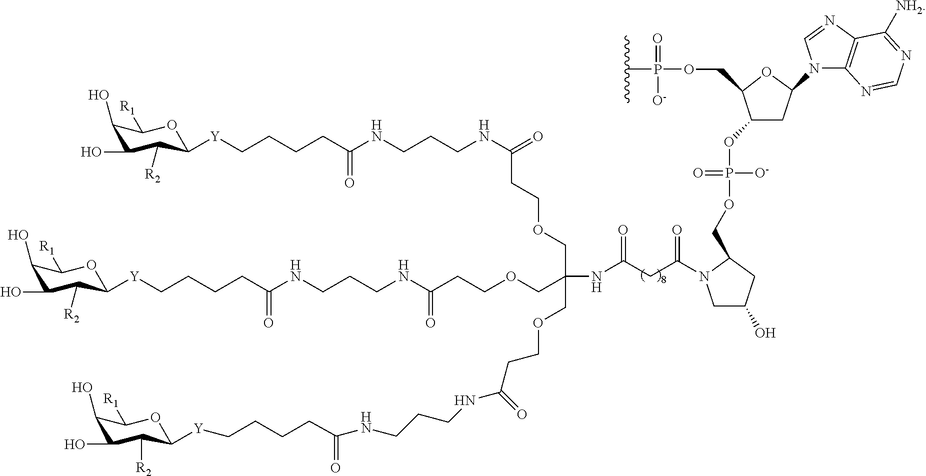

In certain such embodiments, conjugate groups have the following structure:

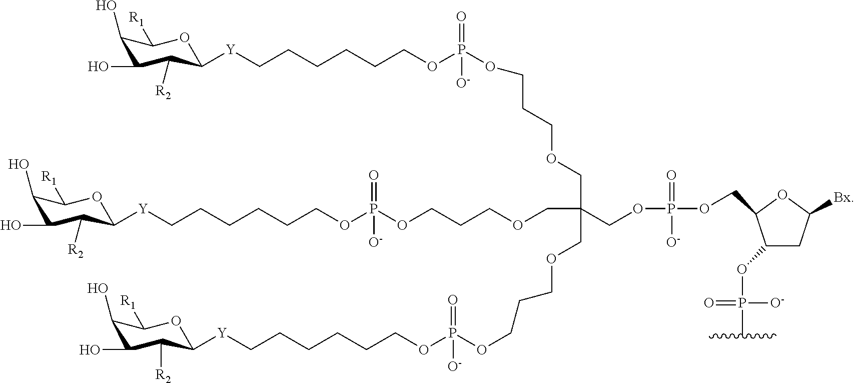

In certain such embodiments, conjugate groups have the following structure:

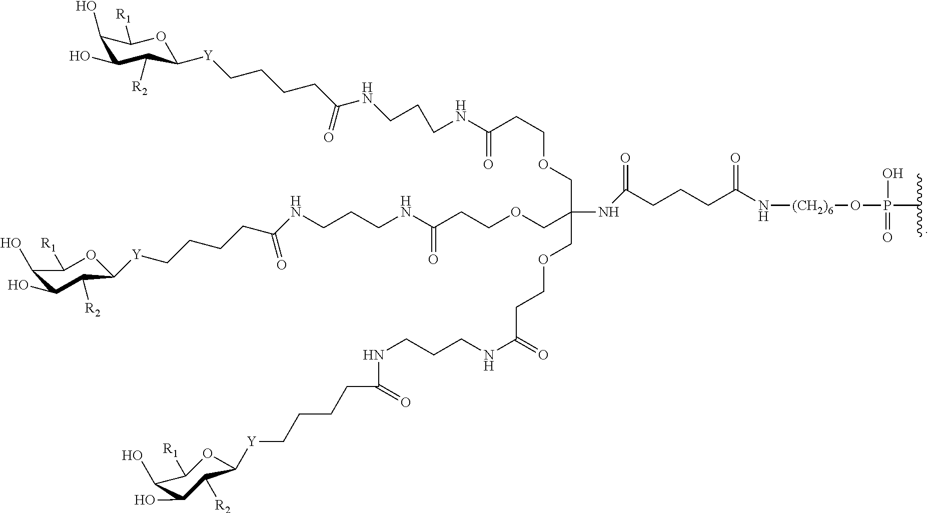

In certain such embodiments, conjugate groups have the following structure:

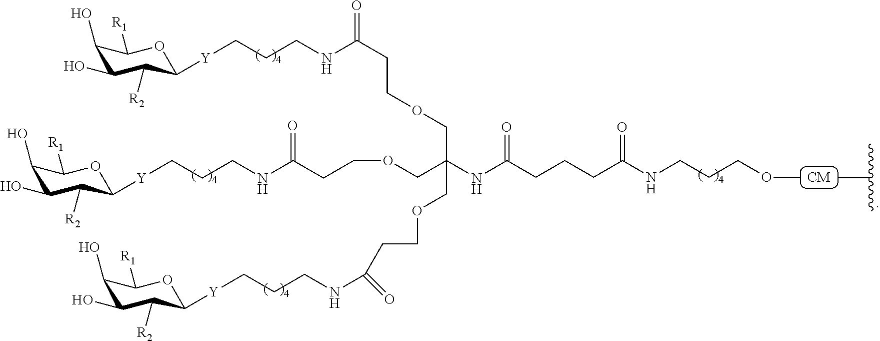

In certain such embodiments, conjugate groups have the following structure:

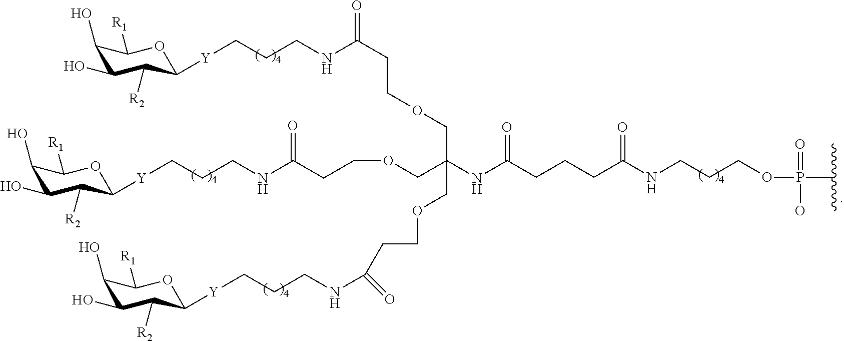

In certain such embodiments, conjugate groups have the following structure:

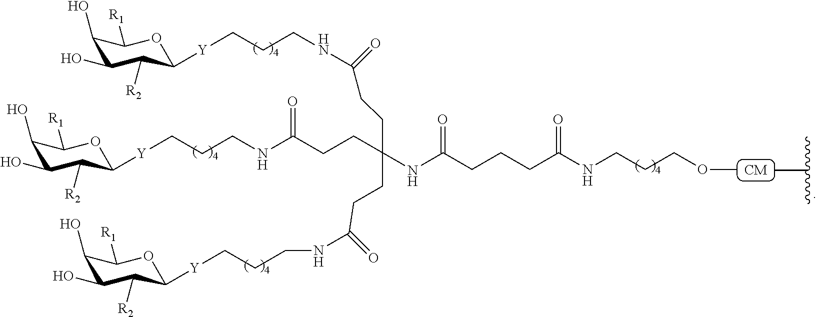

In certain such embodiments, conjugate groups have the following structure:

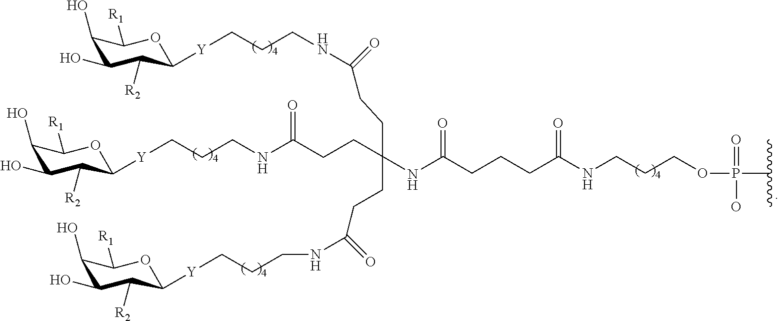

In certain such embodiments, conjugate groups have the following structure:

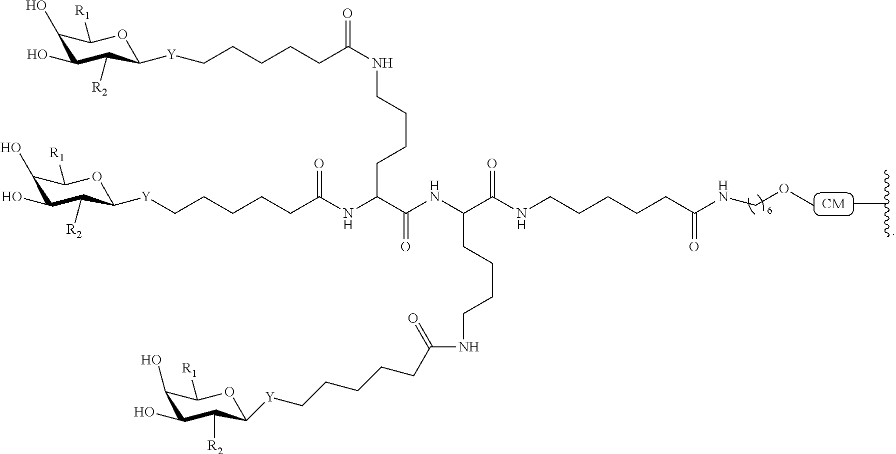

In certain such embodiments, conjugate groups have the following structure:

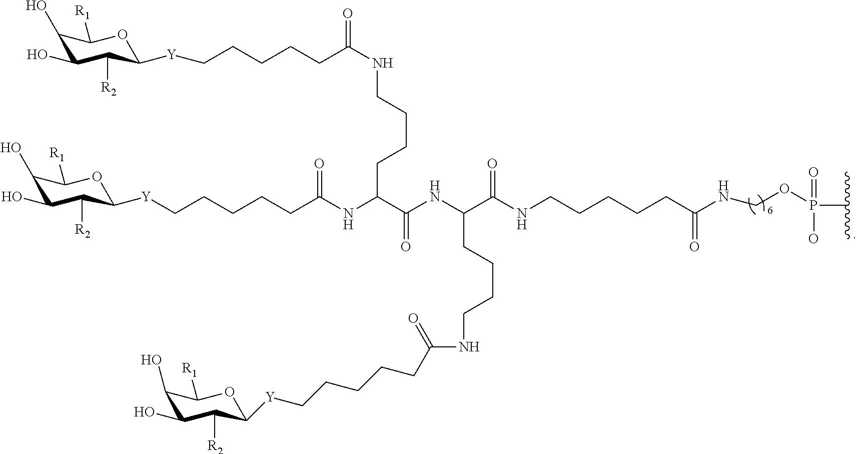

In certain such embodiments, conjugate groups have the following structure:

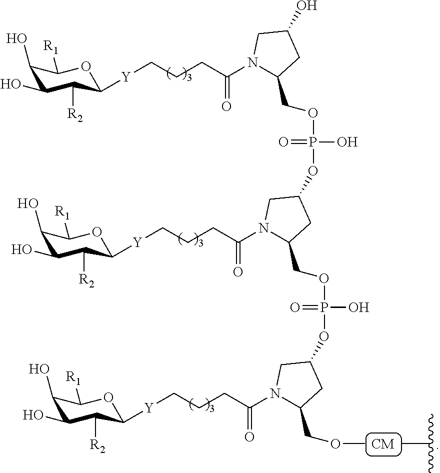

In certain such embodiments, conjugate groups have the following structure:

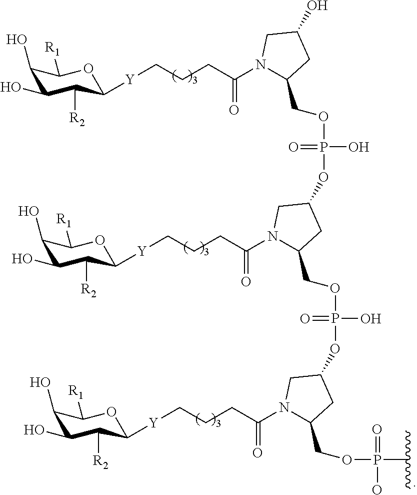

In certain embodiments, the cell-targeting moiety of the conjugate group has the following structure:

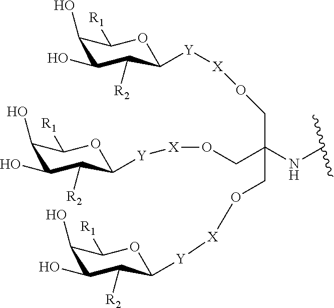

wherein X is a substituted or unsubstituted tether of six to eleven consecutively bonded atoms.

In certain embodiments, the cell-targeting moiety of the conjugate group has the following structure:

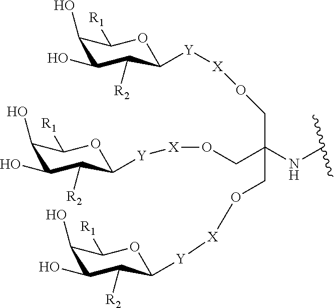

wherein X is a substituted or unsubstituted tether of ten consecutively bonded atoms.

In certain embodiments, the cell-targeting moiety of the conjugate group has the following structure:

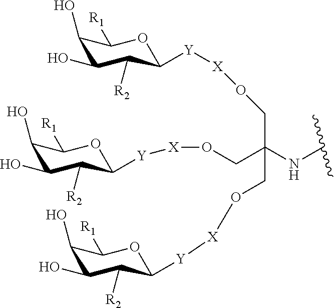

wherein X is a substituted or unsubstituted tether of four to eleven consecutively bonded atoms and wherein the tether comprises exactly one amide bond.

In certain embodiments, the cell-targeting moiety of the conjugate group has the following structure:

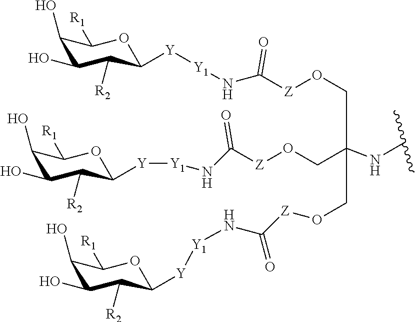

wherein Y₁ and Z are independently selected from a C₁-C₁₂ substituted or unsubstituted alkyl, alkenyl, or alkynyl group, or a group comprising an ether, a ketone, an amide, an ester, a carbamate, an amine, a piperidine, a phosphate, a phosphodiester, a phosphorothioate, a triazole, a pyrrolidine, a disulfide, or a thioether.

In certain such embodiments, the cell-targeting moiety of the conjugate group has the following structure:

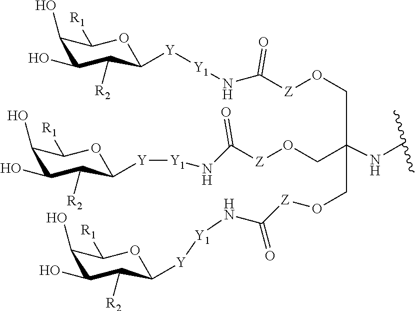

wherein Y₁ and Z are independently selected from a C₁-C₁₂ substituted or unsubstituted alkyl group, or a group comprising exactly one ether or exactly two ethers, an amide, an amine, a piperidine, a phosphate, a phosphodiester, or a phosphorothioate.

In certain such embodiments, the cell-targeting moiety of the conjugate group has the following structure:

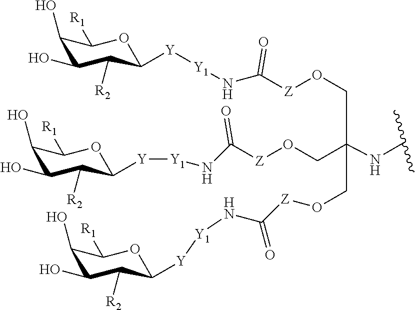

wherein Y₁ and Z are independently selected from a C₁-C₁₂ substituted or unsubstituted alkyl group.

In certain such embodiments, the cell-targeting moiety of the conjugate group has the following structure:

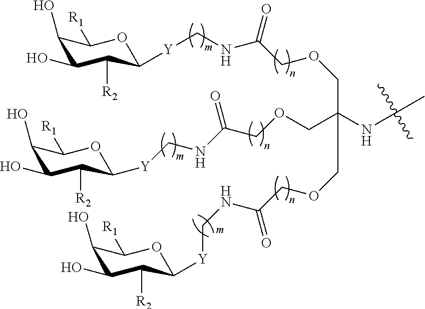

wherein m and n are independently selected from 1, 2, 3, 4, 5, 6, 7, 8, 9, 10, 11, and 12.

In certain such embodiments, the cell-targeting moiety of the conjugate group has the following structure:

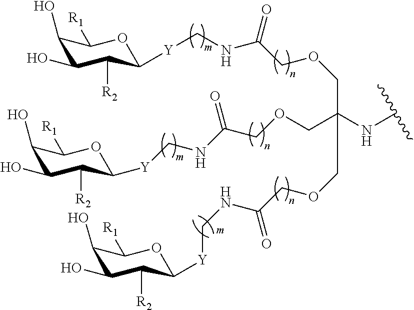

wherein m is 4, 5, 6, 7, or 8, and n is 1, 2, 3, or 4.

In certain embodiments, the cell-targeting moiety of the conjugate group has the following structure:

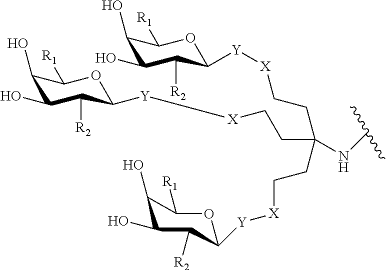

wherein X is a substituted or unsubstituted tether of four to thirteen consecutively bonded atoms, and wherein X does not comprise an ether group.

In certain embodiments, the cell-targeting moiety of the conjugate group has the following structure:

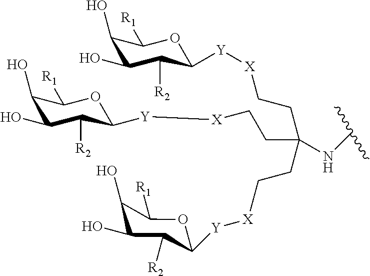

wherein X is a substituted or unsubstituted tether of eight consecutively bonded atoms, and wherein X does not comprise an ether group.

In certain embodiments, the cell-targeting moiety of the conjugate group has the following structure:

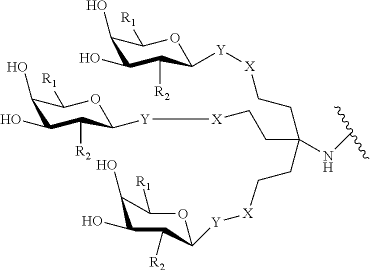

wherein X is a substituted or unsubstituted tether of four to thirteen consecutively bonded atoms, and wherein the tether comprises exactly one amide bond, and wherein X does not comprise an ether group.

In certain embodiments, the cell-targeting moiety of the conjugate group has the following structure:

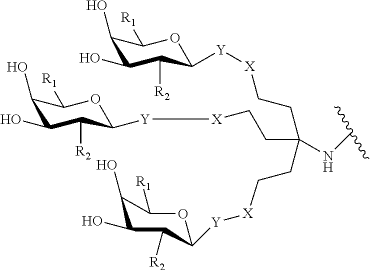

wherein X is a substituted or unsubstituted tether of four to thirteen consecutively bonded atoms and wherein the tether consists of an amide bond and a substituted or unsubstituted C₂-C₁₁ alkyl group.

In certain embodiments, the cell-targeting moiety of the conjugate group has the following structure:

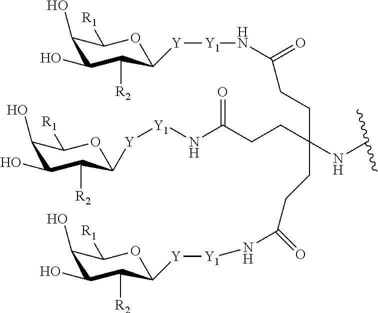

wherein Y₁ is selected from a C₁-C₁₂ substituted or unsubstituted alkyl, alkenyl, or alkynyl group, or a group comprising an ether, a ketone, an amide, an ester, a carbamate, an amine, a piperidine, a phosphate, a phosphodiester, a phosphorothioate, a triazole, a pyrrolidine, a disulfide, or a thioether.

In certain such embodiments, the cell-targeting moiety of the conjugate group has the following structure:

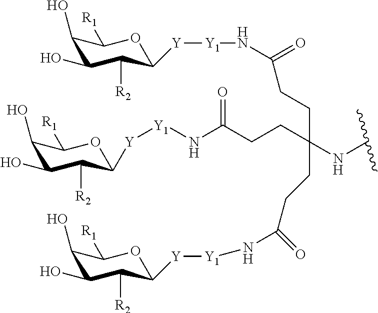

wherein Y₁ is selected from a C₁-C₁₂ substituted or unsubstituted alkyl group, or a group comprising an ether, an amine, a piperidine, a phosphate, a phosphodiester, or a phosphorothioate.

In certain such embodiments, the cell-targeting moiety of the conjugate group has the following structure:

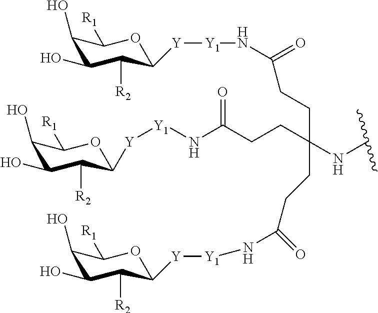

wherein Y₁ is selected from a C₁-C₁₂ substituted or unsubstituted alkyl group.

In certain such embodiments, the cell-targeting moiety of the conjugate group has the following structure:

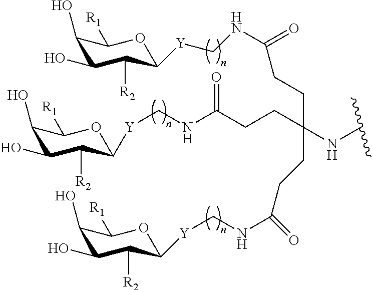

Wherein n is 1, 2, 3, 4, 5, 6, 7, 8, 9, 10, 11, or 12.

In certain such embodiments, the cell-targeting moiety of the conjugate group has the following structure:

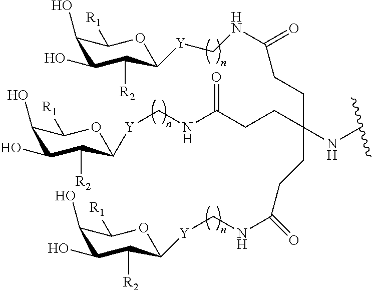

wherein n is 4, 5, 6, 7, or 8.
   h. Certain Conjugated Antisense Compounds

In certain embodiments, a compound has a structure selected from among the following:

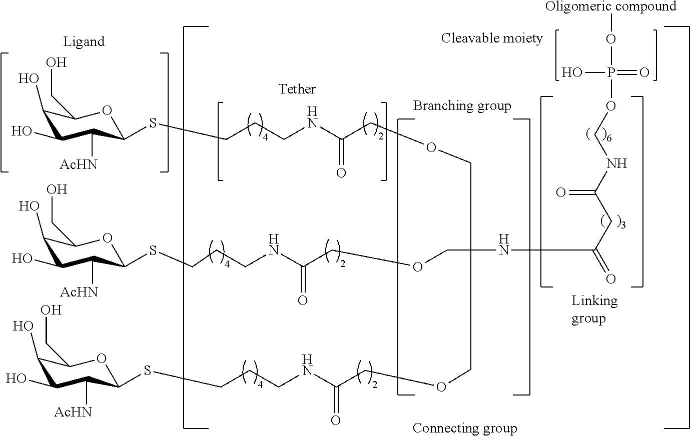

Representative United States patents, United States patent application publications, and international patent application publications that teach the preparation of certain of the above noted conjugates, conjugated antisense compounds, tethers, linkers, branching groups, ligands, cleavable moieties as well as other modifications include without limitation, U.S. Pat. Nos. 5,994,517, 6,300,319, 6,660,720, 6,906,182, 7,262,177, 7,491,805, 8,106,022, 7,723,509, US 2006/0148740, US 2011/0123520, WO 2013/033230 and WO 2012/037254, each of which is incorporated by reference herein in its entirety.

Representative publications that teach the preparation of certain of the above noted conjugates, conjugated antisense compounds, tethers, linkers, branching groups, ligands, cleavable moieties as well as other modifications include without limitation, BIESSEN et al., “The Cholesterol Derivative of a Triantennary Galactoside with High Affinity for the Hepatic Asialoglycoprotein Receptor: a Potent Cholesterol Lowering Agent” J. Med. Chem. (1995) 38:1846-1852, BIESSEN et al., “Synthesis of Cluster Galactosides with High Affinity for the Hepatic Asialoglycoprotein Receptor” J. Med. Chem. (1995) 38:1538-1546, LEE et al., “New and more efficient multivalent glyco-ligands for asialoglycoprotein receptor of mammalian hepatocytes” Bioorganic & Medicinal Chemistry (2011) 19:2494-2500, RENSEN et al., “Determination of the Upper Size Limit for Uptake and Processing of Ligands by the Asialoglycoprotein Receptor on Hepatocytes in Vitro and in Vivo” J. Biol. Chem. (2001) 276(40):37577-37584, RENSEN et al., “Design and Synthesis of Novel N-Acetylgalactosamine-Terminated Glycolipids for Targeting of Lipoproteins to the Hepatic Asialoglycoprotein Receptor” J. Med. Chem. (2004) 47:5798-5808, SLIEDREGT et al., “Design and Synthesis of Novel Amphiphilic Dendritic Galactosides for Selective Targeting of Liposomes to the Hepatic Asialoglycoprotein Receptor” J. Med. Chem. (1999) 42:609-618, and Valentijn et al., “Solid-phase synthesis of lysine-based cluster galactosides with high affinity for the Asialoglycoprotein Receptor” Tetrahedron, 1997, 53(2), 759-770, each of which is incorporated by reference herein in its entirety.

In certain embodiments, conjugated antisense compounds comprise an RNase H based oligonucleotide (such as a gapmer) or a splice modulating oligonucleotide (such as a fully modified oligonucleotide) and any conjugate group comprising at least one, two, or three GalNAc groups. In certain embodiments a conjugated antisense compound comprises any conjugate group found in any of the following references: Lee, Carbohydr Res, 1978, 67, 509-514; Connolly et al., J Biol Chem, 1982, 257, 939-945; Pavia et al., Int J Pep Protein Res, 1983, 22, 539-548; Lee et al., Biochem, 1984, 23, 4255-4261; Lee et al., Glycoconjugate J, 1987, 4, 317-328; Toyokuni et al., Tetrahedron Lett, 1990, 31, 2673-2676; Biessen et al., J Med Chem, 1995, 38, 1538-1546; Valentijn et al., Tetrahedron, 1997, 53, 759-770; Kim et al., Tetrahedron Lett, 1997, 38, 3487-3490; Lee et al., Bioconjug Chem, 1997, 8, 762-765; Kato et al., Glycobiol, 2001, 11, 821-829; Rensen et al., J Biol Chem, 2001, 276, 37577-37584; Lee et al., Methods Enzymol, 2003, 362, 38-43; Westerlind et al., Glycoconj J, 2004, 21, 227-241; Lee et al., Bioorg Med Chem Lett, 2006, 16(19), 5132-5135; Maierhofer et al., Bioorg Med Chem, 2007, 15, 7661-7676; Khorev et al., Bioorg Med Chem, 2008, 16, 5216-5231; Lee et al., Bioorg Med Chem, 2011, 19, 2494-2500; Komilova et al., Analyt Biochem, 2012, 425, 43-46; Pujol et al., Angew Chemie Int Ed Engl, 2012, 51, 7445-7448; Biessen et al., J Med Chem, 1995, 38, 1846-1852; Sliedregt et al., J Med Chem, 1999, 42, 609-618; Rensen et al., J Med Chem, 2004, 47, 5798-5808; Rensen et al., Arterioscler Thromb Vasc Biol, 2006, 26, 169-175; van Rossenberg et al., Gene Ther, 2004, 11, 457-464; Sato et al., J Am Chem Soc, 2004, 126, 14013-14022; Lee et al., J Org Chem, 2012, 77, 7564-7571; Biessen et al., FASEB J, 2000, 14, 1784-1792; Rajur et al., Bioconjug Chem, 1997, 8, 935-940; Duff et al., Methods Enzymol, 2000, 313, 297-321; Maier et al., Bioconjug Chem, 2003, 14, 18-29; Jayaprakash et al., Org Lett, 2010, 12, 5410-5413; Manoharan, Antisense Nucleic Acid Drug Dev, 2002, 12, 103-128; Merwin et al., Bioconjug Chem, 1994, 5, 612-620; Tomiya et al., Bioorg Med Chem, 2013, 21, 5275-5281; International applications WO1998/013381; WO2011/038356; WO1997/046098; WO2008/098788; WO2004/101619; WO2012/037254; WO2011/120053; WO2011/100131; WO2011/163121; WO2012/177947; WO2013/033230; WO2013/075035; WO2012/083185; WO2012/083046; WO2009/082607; WO2009/134487; WO2010/144740; WO2010/148013; WO1997/020563; WO2010/088537; WO2002/043771; WO2010/129709; WO2012/068187; WO2009/126933; WO2004/024757; WO2010/054406; WO2012/089352; WO2012/089602; WO2013/166121; WO2013/165816; U.S. Pat. Nos. 4,751,219; 8,552,163; 6,908,903; 7,262,177; 5,994,517; 6,300,319; 8,106,022; 7,491,805; 7,491,805; 7,582,744; 8,137,695; 6,383,812; 6,525,031; 6,660,720; 7,723,509; 8,541,548; 8,344,125; 8,313,772; 8,349,308; 8,450,467; 8,501,930; 8,158,601; 7,262,177; 6,906,182; 6,620,916; 8,435,491; 8,404,862; 7,851,615; Published U.S. Patent Application Publications US2011/0097264; US2011/0097265; US2013/0004427; US2005/0164235; US2006/0148740; US2008/0281044; US2010/0240730; US2003/0119724; US2006/0183886; US2008/0206869; US2011/0269814; US2009/0286973; US2011/0207799; US2012/0136042; US2012/0165393; US2008/0281041; US2009/0203135; US2012/0035115; US2012/0095075; US2012/0101148; US2012/0128760; US2012/0157509; US2012/0230938; US2013/0109817; US2013/0121954; US2013/0178512; US2013/0236968; US2011/0123520; US2003/0077829; US2008/0108801; and US2009/0203132; each of which is incorporated by reference in its entirety.

### Certain Uses and Features

In certain embodiments, conjugated antisense compounds exhibit potent target RNA reduction in vivo. In certain embodiments, unconjugated antisense compounds accumulate in the kidney. In certain embodiments, conjugated antisense compounds accumulate in the liver. In certain embodiments, conjugated antisense compounds are well tolerated. Such properties render conjugated antisense compounds particularly useful for inhibition of many target RNAs, including, but not limited to those involved in metabolic, cardiovascular and other diseases, disorders or conditions. Thus, provided herein are methods of treating such diseases, disorders or conditions by contacting liver tissues with the conjugated antisense compounds targeted to RNAs associated with such diseases, disorders or conditions. Thus, also provided are methods for ameliorating any of a variety of metabolic, cardiovascular and other diseases, disorders or conditions with the conjugated antisense compounds of the present invention.

In certain embodiments, conjugated antisense compounds are more potent than unconjugated counterpart at a particular tissue concentration. Without wishing to be bound by any theory or mechanism, in certain embodiments, the conjugate may allow the conjugated antisense compound to enter the cell more efficiently or to enter the cell more productively. For example, in certain embodiments conjugated antisense compounds may exhibit greater target reduction as compared to its unconjugated counterpart wherein both the conjugated antisense compound and its unconjugated counterpart are present in the tissue at the same concentrations. For example, in certain embodiments conjugated antisense compounds may exhibit greater target reduction as compared to its unconjugated counterpart wherein both the conjugated antisense compound and its unconjugated counterpart are present in the liver at the same concentrations.

The in vivo data in examples 33 and 34 includes ED₅₀ values for several oligomeric compounds, each comprising the same oligonucleotide targeted to SRB-land one of several different conjugates. As shown below, the conjugate groups in these assays included: (1) GalNAc₃-7ₐ (a 3-sugar GalNAc conjugate group); (2) MP-Triazole-GalNAc₃-7a, also referred to herein as GalNAc₃-33ₐ (the same 3-sugar conjugate group as (1), but with a triazole modification on each GalNAc sugar); (3) GalNAc₁-25ₐ (a 1-sugar GalNAc conjugate group); and (4) GalNAc₁-34ₐ (a 1-sugar GalNAc conjugate that is an analog of GalNAc₁-25ₐ (3), but with a triazole modification on the one GalNac sugar). Structures of GalNAc₃-7ₐ and GalNAc₁-25ₐ are shown in Examples 2 and 11, respectively. Structures of GalNAc₃-33ₐ and GalNAc₁-34ₐ are shown in compounds 148 and 153a (wherein n=6) in Examples 23 and 24, respectively.

In these assays, the unmodified 1-sugar GalNAc conjugate (3) was less active than the 3-sugar unmodified GalNac conjugate (1). Thus, in these assays going from 3 sugars to 1 sugar resulted in a slight decrease in activity. Adding the triazole modification to the 3-sugar unmodified GalNac conjugate (2) did not result in significant additional activity when compared to the unmodified 3-sugar GalNAc conjugate (1). However, the triazole modification on the 1-sugar GalNAc conjugate resulted improved activity compared to the same 1-sugar conjugate lacking the triazole (3). In fact, the triazole-modified 1-sugar GalNAc conjugate had activity comparable to (and perhaps even better than) that of the 3-sugar conjugates. Thus, in these assays, triazole modification of the GalNAc sugar restored the loss of activity observed in reducing the number of sugars in the conjugate from 3 to 1.

| | | Chemistry | |
|---|---|---|---|
| | | (no cleavable | |
| ISIS No. | ED₅₀/# | nucleoside) | Sugar(s) |
| 702489/147 | 3.4/(1) | GalNAc₃-7ₐ | 3 |
| 721456/147 | 3.7/(2) | MP-Triazole- | 3 (modified) |
| | | GalNAc₃-7ₐ | |
| | | (GalNAc₃-33ₐ) | |
| 711462/147 | 4.9/(3) | GalNAc₁-25ₐ | 1 |
| 727852/147 | 2.9/(4) | GalNAc₁-34ₐ | 1 (modified). |

Productive and non-productive uptake of oligonucleotides has been discussed previously (See e.g. Geary, R. S., E. Wancewicz, et al. (2009). “Effect of Dose and Plasma Concentration on Liver Uptake and Pharmacologic Activity of a 2′-Methoxyethyl Modified Chimeric Antisense Oligonucleotide Targeting PTEN.” Biochem. Pharmacol. 78(3): 284-91; & Koller, E., T. M. Vincent, et al. (2011). “Mechanisms of single-stranded phosphorothioate modified antisense oligonucleotide accumulation in hepatocytes.” Nucleic Acids Res. 39(11): 4795-807). Conjugate groups described herein may improve productive uptake.

In certain embodiments, the conjugate groups described herein may further improve potency by increasing the affinity of the conjugated antisense compound for a particular type of cell or tissue. In certain embodiments, the conjugate groups described herein may further improve potency by increasing recognition of the conjugated antisense compound by one or more cell-surface receptors. In certain embodiments, the conjugate groups described herein may further improve potency by facilitating endocytosis of the conjugated antisense compound.

In certain embodiments, the cleavable moiety may further improve potency by allowing the conjugate to be cleaved from the antisense oligonucleotide after the conjugated antisense compound has entered the cell. Accordingly, in certain embodiments, conjugated antisense compounds can be administered at doses lower than would be necessary for unconjugated antisense oligonucleotides.

Phosphorothioate linkages have been incorporated into antisense oligonucleotides previously. Such phosphorothioate linkages are resistant to nucleases and so improve stability of the oligonucleotide. Further, phosphorothioate linkages also bind certain proteins, which results in accumulation of antisense oligonucleotide in the liver. Oligonucleotides with fewer phosphorothioate linkages accumulate less in the liver and more in the kidney (see, for example, Geary, R., “Pharmacokinetic Properties of 2′-O-(2-Methoxyethyl)-Modified Oligonucleotide Analogs in Rats,” Journal of Pharmacology and Experimental Therapeutics, Vol. 296, No. 3, 890-897; & Pharmacological Properties of 2′-O-Methoxyethyl Modified Oligonucleotides in Antisense a Drug Technology, Chapter 10, Crooke, S. T., ed., 2008) In certain embodiments, oligonucleotides with fewer phosphorothioate internucleoside linkages and more phosphodiester internucleoside linkages accumulate less in the liver and more in the kidney. When treating diseases in the liver, this is undesirable for several reasons (1) less drug is getting to the site of desired action (liver); (2) drug is escaping into the urine; and (3) the kidney is exposed to relatively high concentration of drug which can result in toxicities in the kidney. Thus, for liver diseases, phosphorothioate linkages provide important benefits.

In certain embodiments, however, administration of oligonucleotides uniformly linked by phosphoro-thioate internucleoside linkages induces one or more proinflammatory reactions. (see for example: J Lab Clin Med. 1996 September; 128(3):329-38. “Amplification of antibody production by phosphorothioate oligodeoxynucleotides”. Branda et al.; and see also for example: Toxicologic Properties in Antisense a Drug Technology, Chapter 12, pages 342-351, Crooke, S. T., ed., 2008). In certain embodiments, administration of oligonucleotides wherein most of the internucleoside linkages comprise phosphorothioate internucleoside linkages induces one or more proinflammatory reactions.

In certain embodiments, the degree of proinflammatory effect may depend on several variables (e.g. backbone modification, off-target effects, nucleobase modifications, and/or nucleoside modifications) see for example: Toxicologic Properties in Antisense a Drug Technology, Chapter 12, pages 342-351, Crooke, S. T., ed., 2008). In certain embodiments, the degree of proinflammatory effect may be mitigated by adjusting one or more variables. For example the degree of proinflammatory effect of a given oligonucleotide may be mitigated by replacing any number of phosphorothioate internucleoside linkages with phosphodiester internucleoside linkages and thereby reducing the total number of phosphorothioate internucleoside linkages.

In certain embodiments, it would be desirable to reduce the number of phosphorothioate linkages, if doing so could be done without losing stability and without shifting the distribution from liver to kidney. For example, in certain embodiments, the number of phosphorothioate linkages may be reduced by replacing phosphorothioate linkages with phosphodiester linkages. In such an embodiment, the antisense compound having fewer phosphorothioate linkages and more phosphodiester linkages may induce less proinflammatory reactions or no proinflammatory reaction. Although the antisense compound having fewer phosphorothioate linkages and more phosphodiester linkages may induce fewer proinflammatory reactions, the antisense compound having fewer phosphorothioate linkages and more phosphodiester linkages may not accumulate in the liver and may be less efficacious at the same or similar dose as compared to an antisense compound having more phosphorothioate linkages. In certain embodiments, it is therefore desirable to design an antisense compound that has a plurality of phosphodiester bonds and a plurality of phosphorothioate bonds but which also possesses stability and good distribution to the liver.

In certain embodiments, conjugated antisense compounds accumulate more in the liver and less in the kidney than unconjugated counterparts, even when some of the phosphorothioate linkages are replaced with less proinflammatory phosphodiester internucleoside linkages. In certain embodiments, conjugated antisense compounds accumulate more in the liver and are not excreted as much in the urine compared to its unconjugated counterparts, even when some of the phosphorothioate linkages are replaced with less proinflammatory phosphodiester internucleoside linkages. In certain embodiments, the use of a conjugate allows one to design more potent and better tolerated antisense drugs. Indeed, in certain embodiments, conjugated antisense compounds have larger therapeutic indexes than unconjugated counterparts. This allows the conjugated antisense compound to be administered at a higher absolute dose, because there is less risk of proinflammatory response and less risk of kidney toxicity. This higher dose, allows one to dose less frequently, since the clearance (metabolism) is expected to be similar. Further, because the compound is more potent, as described above, one can allow the concentration to go lower before the next dose without losing therapeutic activity, allowing for even longer periods between dosing.

In certain embodiments, the inclusion of some phosphorothioate linkages remains desirable. For example, the terminal linkages are vulnerable to exonucleases and so in certain embodiments, those linkages are phosphorothioate or other modified linkage. Internucleoside linkages linking two deoxynucleosides are vulnerable to endonucleases and so in certain embodiments those linkages are phosphorothioate or other modified linkage. Internucleoside linkages between a modified nucleoside and a deoxynucleoside where the deoxynucleoside is on the 5′ side of the linkage deoxynucleosides are vulnerable to endonucleases and so in certain embodiments those linkages are phosphorothioate or other modified linkage. Internucleoside linkages between two modified nucleosides of certain types and between a deoxynucleoside and a modified nucleoside of certain type where the modified nucleoside is at the 5′ side of the linkage are sufficiently resistant to nuclease digestion, that the linkage can be phosphodiester.

In certain embodiments, the antisense oligonucleotide of a conjugated antisense compound comprises fewer than 16 phosphorothioate linkages. In certain embodiments, the antisense oligonucleotide of a conjugated antisense compound comprises fewer than 15 phosphorothioate linkages. In certain embodiments, the antisense oligonucleotide of a conjugated antisense compound comprises fewer than 14 phosphorothioate linkages. In certain embodiments, the antisense oligonucleotide of a conjugated antisense compound comprises fewer than 13 phosphorothioate linkages. In certain embodiments, the antisense oligonucleotide of a conjugated antisense compound comprises fewer than 12 phosphorothioate linkages. In certain embodiments, the antisense oligonucleotide of a conjugated antisense compound comprises fewer than 11 phosphorothioate linkages. In certain embodiments, the antisense oligonucleotide of a conjugated antisense compound comprises fewer than 10 phosphorothioate linkages. In certain embodiments, the antisense oligonucleotide of a conjugated antisense compound comprises fewer than 9 phosphorothioate linkages. In certain embodiments, the antisense oligonucleotide of a conjugated antisense compound comprises fewer than 8 phosphorothioate linkages.

In certain embodiments, antisense compounds comprising one or more conjugate group described herein has increased activity and/or potency and/or tolerability compared to a parent antisense compound lacking such one or more conjugate group. Accordingly, in certain embodiments, attachment of such conjugate groups to an oligonucleotide is desirable. Such conjugate groups may be attached at the 5′-, and/or 3′-end of an oligonucleotide. In certain instances, attachment at the 5′-end is synthetically desirable. Typically, oligonucleotides are synthesized by attachment of the 3′ terminal nucleoside to a solid support and sequential coupling of nucleosides from 3′ to 5′ using techniques that are well known in the art. Accordingly if a conjugate group is desired at the 3′-terminus, one may (1) attach the conjugate group to the 3′-terminal nucleoside and attach that conjugated nucleoside to the solid support for subsequent preparation of the oligonucleotide or (2) attach the conjugate group to the 3′-terminal nucleoside of a completed oligonucleotide after synthesis. Neither of these approaches is very efficient and thus both are costly. In particular, attachment of the conjugated nucleoside to the solid support, while demonstrated in the Examples herein, is an inefficient process. In certain embodiments, attaching a conjugate group to the 5′-terminal nucleoside is synthetically easier than attachment at the 3′-end. One may attach a non-conjugated 3′ terminal nucleoside to the solid support and prepare the oligonucleotide using standard and well characterized reactions. One then needs only to attach a 5′nucleoside having a conjugate group at the final coupling step. In certain embodiments, this is more efficient than attaching a conjugated nucleoside directly to the solid support as is typically done to prepare a 3′-conjugated oligonucleotide. The Examples herein demonstrate attachment at the 5′-end. In addition, certain conjugate groups have synthetic advantages. For Example, certain conjugate groups comprising phosphorus linkage groups are synthetically simpler and more efficiently prepared than other conjugate groups, including conjugate groups reported previously (e.g., WO/2012/037254).

In certain embodiments, conjugated antisense compounds are administered to a subject. In such embodiments, antisense compounds comprising one or more conjugate group described herein has increased activity and/or potency and/or tolerability compared to a parent antisense compound lacking such one or more conjugate group. Without being bound by mechanism, it is believed that the conjugate group helps with distribution, delivery, and/or uptake into a target cell or tissue. In certain embodiments, once inside the target cell or tissue, it is desirable that all or part of the conjugate group to be cleaved to release the active oligonucleotide. In certain embodiments, it is not necessary that the entire conjugate group be cleaved from the oligonucleotide. For example, in Example 32 a conjugated oligonucleotide was administered to mice and a number of different chemical species, each comprising a different portion of the conjugate group remaining on the oligonucleotide, were detected (Table 32). This conjugated antisense compound demonstrated good potency (Table 31). Thus, in certain embodiments, such metabolite profile of multiple partial cleavage of the conjugate group does not interfere with activity/potency. Nevertheless, in certain embodiments it is desirable that a prodrug (conjugated oligonucleotide) yield a single active compound. In certain instances, if multiple forms of the active compound are found, it may be necessary to determine relative amounts and activities for each one. In certain embodiments where regulatory review is required (e.g., USFDA or counterpart) it is desirable to have a single (or predominantly single) active species. In certain such embodiments, it is desirable that such single active species be the antisense oligonucleotide lacking any portion of the conjugate group. In certain embodiments, conjugate groups at the 5′-end are more likely to result in complete metabolism of the conjugate group. Without being bound by mechanism it may be that endogenous enzymes responsible for metabolism at the 5′ end (e.g., 5′ nucleases) are more active/efficient than the 3′ counterparts. In certain embodiments, the specific conjugate groups are more amenable to metabolism to a single active species. In certain embodiments, certain conjugate groups are more amenable to metabolism to the oligonucleotide.

### D. Antisense

In certain embodiments, oligomeric compounds of the present invention are antisense compounds. In such embodiments, the oligomeric compound is complementary to a target nucleic acid. In certain embodiments, a target nucleic acid is an RNA. In certain embodiments, a target nucleic acid is a non-coding RNA. In certain embodiments, a target nucleic acid encodes a protein. In certain embodiments, a target nucleic acid is selected from a mRNA, a pre-mRNA, a microRNA, a non-coding RNA, including small non-coding RNA, and a promoter-directed RNA. In certain embodiments, oligomeric compounds are at least partially complementary to more than one target nucleic acid. For example, oligomeric compounds of the present invention may be microRNA mimics, which typically bind to multiple targets.

In certain embodiments, antisense compounds comprise a portion having a nucleobase sequence at least 70% complementary to the nucleobase sequence of a target nucleic acid. In certain embodiments, antisense compounds comprise a portion having a nucleobase sequence at least 80% complementary to the nucleobase sequence of a target nucleic acid. In certain embodiments, antisense compounds comprise a portion having a nucleobase sequence at least 90% complementary to the nucleobase sequence of a target nucleic acid. In certain embodiments, antisense compounds comprise a portion having a nucleobase sequence at least 95% complementary to the nucleobase sequence of a target nucleic acid. In certain embodiments, antisense compounds comprise a portion having a nucleobase sequence at least 98% complementary to the nucleobase sequence of a target nucleic acid. In certain embodiments, antisense compounds comprise a portion having a nucleobase sequence that is 100% complementary to the nucleobase sequence of a target nucleic acid. In certain embodiments, antisense compounds are at least 70%, 80%, 90%, 95%, 98%, or 100% complementary to the nucleobase sequence of a target nucleic acid over the entire length of the antisense compound.

Antisense mechanisms include any mechanism involving the hybridization of an oligomeric compound with target nucleic acid, wherein the hybridization results in a biological effect. In certain embodiments, such hybridization results in either target nucleic acid degradation or occupancy with concomitant inhibition or stimulation of the cellular machinery involving, for example, translation, transcription, or polyadenylation of the target nucleic acid or of a nucleic acid with which the target nucleic acid may otherwise interact.

One type of antisense mechanism involving degradation of target RNA is RNase H mediated antisense. RNase H is a cellular endonuclease which cleaves the RNA strand of an RNA:DNA duplex. It is known in the art that single-stranded antisense compounds which are “DNA-like” elicit RNase H activity in mammalian cells. Activation of RNase H, therefore, results in cleavage of the RNA target, thereby greatly enhancing the efficiency of DNA-like oligonucleotide-mediated inhibition of gene expression.

Antisense mechanisms also include, without limitation RNAi mechanisms, which utilize the RISC pathway. Such RNAi mechanisms include, without limitation siRNA, ssRNA and microRNA mechanisms. Such mechanisms include creation of a microRNA mimic and/or an anti-microRNA.

Antisense mechanisms also include, without limitation, mechanisms that hybridize or mimic non-coding RNA other than microRNA or mRNA. Such non-coding RNA includes, but is not limited to promoter-directed RNA and short and long RNA that effects transcription or translation of one or more nucleic acids.

In certain embodiments, oligonucleotides comprising conjugates described herein are RNAi compounds. In certain embodiments, oligomeric oligonucleotides comprising conjugates described herein are ssRNA compounds. In certain embodiments, oligonucleotides comprising conjugates described herein are paired with a second oligomeric compound to form an siRNA. In certain such embodiments, the second oligomeric compound also comprises a conjugate. In certain embodiments, the second oligomeric compound is any modified or unmodified nucleic acid. In certain embodiments, the oligonucleotides comprising conjugates described herein is the antisense strand in an siRNA compound. In certain embodiments, the oligonucleotides comprising conjugates described herein is the sense strand in an siRNA compound. In embodiments in which the conjugated oligomeric compound is double-stranded siRNA, the conjugate may be on the sense strand, the antisense strand or both the sense strand and the antisense strand.

### E. Target Nucleic Acids, Regions and Segments

In certain embodiments, conjugated antisense compounds target any nucleic acid. In certain embodiments, the target nucleic acid encodes a target protein that is clinically relevant. In such embodiments, modulation of the target nucleic acid results in clinical benefit. Certain target nucleic acids include, but are not limited to, the target nucleic acids illustrated in Table 1.

TABLE 1
Certain Target Nucleic Acids

| | | GENBANK ® Accession | SEQ ID |
|---|---|---|---|
| Target | Species | Number | NO |
| Androgen | Human | NT_011669.17 truncated | 1 |
| Receptor (AR) | | from nucleobases | |
| | | 5079000 to 5270000 | |
| Apolipoprotein | Human | NM_005577.2 | 2 |
| (a) (Apo(a)) | | | |
| Apolipoprotein | Human | NM_000384.1 | 3 |
| B (ApoB) | | | |
| Apolipoprotein | Human | NT_033899.8 truncated | 4 |
| C-III (ApoCIII) | | from nucleobases | |
| | | 20262640 to 20266603 | |
| Apolipoprotein | Human | NM_000040.1 | 5 |
| C-III (ApoCIII) | | | |
| C-Reactive | Human | M11725.1 | 6 |
| Protein (CRP) | | | |
| eIF4E | Human | M15353.1 | 7 |
| Factor VII | Human | NT_027140.6 truncated | 8 |
| | | from nucleobases | |
| | | 1255000 to 1273000 | |
| Factor XI | Human | NM_000128.3 | 9 |
| Glucocorticoid | Human | the complement | 10 |
| Receptor (GCCR) | | NT_029289.10 truncated | |
| | | from nucleobases | |
| | | 3818000 to 3980000 | |
| Glucagon | Human | NW_926918.1 truncated | 11 |
| Receptor (GCGR) | | from nucleobases | |
| | | 16865000 to 16885000 | |
| HBV | Human | U95551.1 | 12 |
| Protein Tyrosine | Human | NM_002827.2 | 13 |
| Phosphatase 1B | | | |
| (PTP1B) | | | |
| Protein Tyrosine | Human | NT_011362.9 truncated | 14 |
| Phosphatase 1B | | from nucleobases | |
| (PTP1B) | | 14178000 to 14256000 | |
| STAT3 | Human | NM_139276.2 | 15 |
| Transthyretin | Human | NM_000371.3 | 16 |
| (TTR) | | | |

The targeting process usually includes determination of at least one target region, segment, or site within the target nucleic acid for the antisense interaction to occur such that the desired effect will result.

In certain embodiments, a target region is a structurally defined region of the nucleic acid. For example, in certain such embodiments, a target region may encompass a 3′ UTR, a 5′ UTR, an exon, an intron, a coding region, a translation initiation region, translation termination region, or other defined nucleic acid region or target segment.

In certain embodiments, a target segment is at least about an 8-nucleobase portion of a target region to which a conjugated antisense compound is targeted. Target segments can include DNA or RNA sequences that comprise at least 8 consecutive nucleobases from the 5-terminus of one of the target segments (the remaining nucleobases being a consecutive stretch of the same DNA or RNA beginning immediately upstream of the 5′-terminus of the target segment and continuing until the DNA or RNA comprises about 8 to about 30 nucleobases). Target segments are also represented by DNA or RNA sequences that comprise at least 8 consecutive nucleobases from the 3′-terminus of one of the target segments (the remaining nucleobases being a consecutive stretch of the same DNA or RNA beginning immediately downstream of the 3′-terminus of the target segment and continuing until the DNA or RNA comprises about 8 to about 30 nucleobases). Target segments can also be represented by DNA or RNA sequences that comprise at least 8 consecutive nucleobases from an internal portion of the sequence of a target segment, and may extend in either or both directions until the conjugated antisense compound comprises about 8 to about 30 nucleobases.

In certain embodiments, antisense compounds targeted to the nucleic acids listed in Table 1 can be modified as described herein. In certain embodiments, the antisense compounds can have a modified sugar moiety, an unmodified sugar moiety or a mixture of modified and unmodified sugar moieties as described herein. In certain embodiments, the antisense compounds can have a modified internucleoside linkage, an unmodified internucleoside linkage or a mixture of modified and unmodified internucleoside linkages as described herein. In certain embodiments, the antisense compounds can have a modified nucleobase, an unmodified nucleobase or a mixture of modified and unmodified nucleobases as described herein. In certain embodiments, the antisense compounds can have a motif as described herein. In certain embodiments, antisense compounds targeted to the nucleic acids listed in Table 1 can be conjugated as described herein.

### 1. Androgen Receptor (AR)

AR is a transcription factor implicated as a driver of prostate cancer. AR is activated by binding to its hormone ligands: androgen, testosterone, and/or DHT. Androgen deprivation therapy, also known as “chemical castration,” is a first-line treatment strategy against hormone-sensitive, androgen-dependent prostate cancer that reduces circulating androgen levels and thereby inhibits AR activity. However, androgen deprivation therapy frequently leads to the emergence and growth of “castration-resistant” advanced prostate cancer, in which AR signaling is reactivated independent of ligand binding. The mechanisms underlying castration resistance in advanced prostate cancer remain unclear.

### Certain Conjugated Antisense Compounds Targeted to an AR Nucleic Acid

In certain embodiments, conjugated antisense compounds are targeted to an AR nucleic acid having the sequence of GENBANK® Accession No. NT_011669.17 nucleobases 5079000 to 5270000, incorporated herein as SEQ ID NO: 1. In certain such embodiments, a conjugated antisense compound is at least 90%, at least 950%, or 100% complementary to SEQ ID NO: 1.

In certain embodiments, a conjugated antisense compound targeted to SEQ ID NO: 1 comprises an at least 8 consecutive nucleobase sequence selected from the nucleobase sequence of any of SEQ ID NOs: 17-24. In certain embodiments, a conjugated antisense compound targeted to SEQ ID NO: 1 comprises a nucleobase sequence selected from the nucleobase sequence of any of SEQ ID NOs: 17-24. In certain embodiments, such conjugated antisense compounds comprise a conjugate comprising 1-3 GalNAc ligands. In certain embodiments, such antisense compounds comprise a conjugate disclosed herein.

TABLE 2
Antisense Compounds Targeted to AR SEQ ID NO: 1

| | Target | | | SEQ |
|---|---|---|---|---|
| | Start | | | ID |
| ISIS No | Site | Sequence | Motif | NO |
| 560131 | 58721 | TTGATTTAATGGTTGC | kkkdddddddddkkke | 17 |
| | 58751 | | | |
| 569213 | 58720 | TGATTTAATGGTTGCA | kkkdddddddddkkke | 18 |
| | 58750 | | | |
| 569216 | 58720 | TGATTTAATGGTTGCA | ekkkddddddddkkke | 18 |
| | 58750 | | | |
| 569221 | 58720 | TGATTTAATGGTTGCA | eekkkddddddddkkk | 18 |
| | 58750 | | | |
| 569236 | 58720 | TGATTTAATGGTTGCA | ekkkdddddddkkkee | 18 |
| | 58750 | | | |
| 579671 | 58721 | TTGATTTAATGGTTGC | ekkekkdddddddkkk | 17 |
| | 58751 | | | |
| 586124 | 58719 | GATTTAATGGTTGCAA | kkkddddddddddkkk | 19 |
| 583918 | 5052 | AGTCGCGACTCTGGTA | kkkddddddddddkkk | 20 |
| 584149 | 8638 | GTCAATATCAAAGCAC | kkkddddddddddkkk | 21 |
| 584163 | 11197 | GAACATTATTAGGCTA | kkkddddddddddkkk | 22 |
| 584269 | 40615 | CCTTATGGATGCTGCT | kkkddddddddddkkk | 23 |
| 584468 | 115272 | CATTGTACTATGCCAG | kkkddddddddddkkk | 24 |

### AR Therapeutic Indications

In certain embodiments, the invention provides methods for using a conjugated antisense compound targeted to an AR nucleic acid for modulating the expression of AR in a subject. In certain embodiments, the expression of AR is reduced.

In certain embodiments, the invention provides methods for using a conjugated antisense compound targeted to an AR nucleic acid in a pharmaceutical composition for treating a subject. In certain embodiments, the subject has prostate cancer, such as castration-resistant prostate cancer. In certain embodiments, the subject has prostate cancer resistant to a diarylhydantoin Androgen Receptor (AR) inhibitor, such as MDV3100, which is also known as Enzalutamide. MDV3100 or Enzalutamide is an experimental androgen receptor antagonist drug developed by Medivation for the treatment of castration-resistant prostate cancer. In certain embodiments, the subject has breast cancer. In certain aspects, the subject's breast cancer can have one or more of the following characteristics: Androgen Receptor positive, dependent on androgen for growth, Estrogen Receptor (ER) negative, independent of estrogen for growth, Progesterone Receptor (PR) negative, independent of progesterone for growth, or Her2/neu negative. In certain aspects, the breast cancer or breast cancer cell is apocrine.

In certain embodiments, the invention provides methods for using a conjugated antisense compound targeted to an AR nucleic acid in the preparation of a medicament.

### 2. Apolipoprotein (a) (Apo(a))

One Apo(a) protein is linked via a disulfide bond to a single ApoB protein to form a lipoprotein(a) (Lp(a)) particle. The Apo(a) protein shares a high degree of homology with plasminogen particularly within the kringle IV type 2 repetitive domain. It is thought that the kringle repeat domain in Apo(a) may be responsible for its pro-thrombotic and anti-fibrinolytic properties, potentially enhancing atherosclerotic progression. Apo(a) is transcriptionally regulated by IL-6 and in studies in rheumatoid arthritis patients treated with an IL-6 inhibitor (tocilizumab), plasma levels were reduced by 30% after 3 month treatment. Apo(a) has been shown to preferentially bind oxidized phospholipids and potentiate vascular inflammation. Further, studies suggest that the Lp(a) particle may also stimulate endothelial permeability, induce plasminogen activator inhibitor type-1 expression and activate macrophage interleukin-8 secretion. Importantly, recent genetic association studies revealed that Lp(a) was an independent risk factor for myocardial infarction, stroke, peripheral vascular disease and abdominal aortic aneurysm. Further, in the Precocious Coronary Artery Disease (PROCARDIS) study, Clarke et al. described robust and independent associations between coronary heart disease and plasma Lp(a) concentrations. Additionally, Solfrizzi et al., suggested that increased serum Lp(a) may be linked to an increased risk for Alzheimer's Disease (AD). Antisense compounds targeting Apo(a) have been previously disclosed in WO2005/000201 and U.S. 61/651,539, herein incorporated by reference in its entirety. An antisense oligonucleotide targeting Apo(a), ISIS-APOA_{Rx}, is currently in a Phase I clinical trial to study its safety profile.

### Certain Conjugated Antisense Compounds Targeted to an Apo(a) Nucleic Acid

In certain embodiments, conjugated antisense compounds are targeted to an Apo(a) nucleic acid having the sequence of GENBANK® Accession No. NM_005577.2, incorporated herein as SEQ ID NO: 2. In certain such embodiments, a conjugated antisense compound is at least 90%, at least 95%, or 100% complementary to SEQ ID NO: 2.

In certain embodiments, a conjugated antisense compound targeted to SEQ ID NO: 2 comprises an at least 8 consecutive nucleobase sequence selected from the nucleobase sequence ofany of SEQ ID NOs: 25-30. In certain embodiments, a conjugated antisense compound targeted to SEQ ID NO: 2 comprises a nucleobase sequence selected from the nucleobase sequence of any of SEQ ID NOs: 25-30. In certain embodiments, such conjugated antisense compounds comprise a conjugate comprising 1-3 GalNAc ligands. In certain embodiments, such antisense compounds comprise a conjugate disclosed herein.

TABLE 3
Antisense Compounds targeted to Apo(a)
SEQ ID NO: 2

| | Target | | | SEQ |
|---|---|---|---|---|
| ISIS | Start | | | ID |
| No | Site | Sequence (5′-3′) | Motif | NO |
| 494372 | 3901 | TGCTCCGTTGGTGCTTGTTC | eeeeeddddd | 25 |
| | | | dddddeeeee | |
| 494283 | 584 | TCTTCCTGTGACAGTGGTGG | eeeeeddddd | 26 |
| | 926 | | dddddeeeee | |
| | 1610 | | | |
| | 1952 | | | |
| | 2294 | | | |
| | 3320 | | | |
| 494284 | 585 | TTCTTCCTGTGACAGTGGTG | eeeeeddddd | 27 |
| | 927 | | dddddeeeee | |
| | 1611 | | | |
| | 1953 | | | |
| | 2295 | | | |
| | 3321 | | | |
| 494286 | 587 | GGTTCTTCCTGTGACAGTGG | eeeeeddddd | 28 |
| | 929 | | dddddeeeee | |
| | 1613 | | | |
| | 1955 | | | |
| | 2297 | | | |
| 494301 | 628 | CGACTATGCGAGTGTGGTGT | eeeeeddddd | 29 |
| | 970 | | dddddeeeee | |
| | 1312 | | | |
| | 1654 | | | |
| | 1996 | | | |
| | 2338 | | | |
| | 2680 | | | |
| | 3022 | | | |
| 494302 | 629 | CCGACTATGCGAGTGTGGTG | eeeeeddddd | 30 |
| | 971 | | dddddeeeee | |
| | 1313 | | | |
| | 1655 | | | |
| | 1997 | | | |
| | 2339 | | | |
| | 2681 | | | |
| | 3023 | | | |

### Apo(a) Therapeutic Indications

In certain embodiments, the invention provides methods for using a conjugated antisense compound targeted to an Apo(a) nucleic acid for modulating the expression of Apo(a) in a subject. In certain embodiments, the expression of Apo(a) is reduced.

In certain embodiments, the invention provides methods for using a conjugated antisense compound targeted to an Apo(a) nucleic acid in a pharmaceutical composition for treating a subject. In certain embodiments, the subject has a cardiovascular and/or metabolic disease, disorder or condition. In certain embodiments, the subject has hypercholesterolemia, non-familial hypercholesterolemia, familial hypercholesterolemia, heterozygous familial hypercholesterolemia, homozygous familial hypercholesterolemia, mixed dyslipidemia, atherosclerosis, a risk of developing atherosclerosis, coronary heart disease, a history of coronary heart disease, early onset coronary heart disease, one or more risk factors for coronary heart disease, type II diabetes, type II diabetes with dyslipidemia, dyslipidemia, hypertriglyceridemia, hyperlipidemia, hyperfattyacidemia, hepatic steatosis, non-alcoholic steatohepatitis, and/or non-alcoholic fatty liver disease.

In certain embodiments, the invention provides methods for using a conjugated antisense compound targeted to an Apo(a) nucleic acid in the preparation of a medicament.

### 3. Apolipoprotein B (ApoB)

ApoB (also known as apolipoprotein B-100; ApoB-100, apolipoprotein B-48; ApoB-48 and Ag(x) antigen), is a large glycoprotein that serves an indispensable role in the assembly and secretion of lipids and in the transport and receptor-mediated uptake and delivery of distinct classes of lipoproteins. ApoB performs a variety of activities, from the absorption and processing of dietary lipids to the regulation of circulating lipoprotein levels (Davidson and Shelness, Annu. Rev. Nutr., 2000, 20, 169-193). This latter property underlies its relevance in terms of atherosclerosis susceptibility, which is highly correlated with the ambient concentration of ApoB-containing lipoproteins (Davidson and Shelness, Annu. Rev. Nutr., 2000, 20, 169-193). ApoB-100 is the major protein component of LDL-C and contains the domain required for interaction of this lipoprotein species with the LDL receptor. Elevated levels of LDL-C are a risk factor for cardiovascular disease, including atherosclerosis. Antisense compounds targeting ApoB have been previously disclosed in WO2004/044181, herein incorporated by reference in its entirety. An antisense oligonucleotide targeting ApoB, KYNAMRO™, has been approved by the U.S. Food and Drug Administration (FDA) as an adjunct treatment to lipid-lowering medications and diet to reduce low density lipoprotein-cholesterol (LDL-C), ApoB, total cholesterol (TC), and non-high density lipoprotein-cholesterol (non HDL-C) in patients with homozygous familial hypercholesterolemia (HoFH). However, there is still a need to provide patients with additional and more potent treatment options.

### Certain Conjugated Antisense Compounds Targeted to an ApoB Nucleic Acid

In certain embodiments, conjugated antisense compounds are targeted to an ApoB nucleic acid having the sequence of GENBANK® Accession No. NM_000384.1, incorporated herein as SEQ ID NO: 3. In certain such embodiments, a conjugated antisense compound is at least 90%, at least 95%, or 100% complementary to SEQ ID NO: 3.

In certain embodiments, a conjugated antisense compound targeted to SEQ ID NO: 3 comprises an at least 8 consecutive nucleobase sequence of SEQ ID NO: 31. In certain embodiments, a conjugated antisense compound targeted to SEQ ID NO: 3 comprises a nucleobase sequence of SEQ ID NO: 31. In certain embodiments, such conjugated antisense compounds comprise a conjugate comprising 1-3 GalNAc ligands. In certain embodiments, such antisense compounds comprise a conjugate disclosed herein.

TABLE 4
Antisense Compounds targeted to ApoB SEQ ID NO: 3

| | Target | | | |
|---|---|---|---|---|
| | Start | Sequence | | SEQ ID |
| ISIS No | Site | (5′-3′) | Motif | NO |
| 301012 | 3249 | GCCTCAGTCT | eeeeeddddd | 31 |
| | | GCTTCGCACC | dddddeeeee | |

### ApoB Therapeutic Indications

In certain embodiments, the invention provides methods for using a conjugated antisense compound targeted to an ApoB nucleic acid for modulating the expression of ApoB in a subject. In certain embodiments, the expression of ApoB is reduced.

In certain embodiments, the invention provides methods for using a conjugated antisense compound targeted to an ApoB nucleic acid in a pharmaceutical composition for treating a subject. In certain embodiments, the subject has a cardiovascular and/or metabolic disease, disorder or condition. In certain embodiments, the subject has hypercholesterolemia, non-familial hypercholesterolemia, familial hypercholesterolemia, heterozygous familial hypercholesterolemia, homozygous familial hypercholesterolemia, mixed dyslipidemia, atherosclerosis, a risk of developing atherosclerosis, coronary heart disease, a history of coronary heart disease, early onset coronary heart disease, one or more risk factors for coronary heart disease, type II diabetes, type II diabetes with dyslipidemia, dyslipidemia, hypertriglyceridemia, hyperlipidemia, hyperfattyacidemia, hepatic steatosis, non-alcoholic steatohepatitis, and/or non-alcoholic fatty liver disease.

In certain embodiments, the invention provides methods for using a conjugated antisense compound targeted to an ApoB nucleic acid in the preparation of a medicament.

### 4. Apolipoprotein C-III (ApoCIII)

ApoCIII is a constituent of HDL and of triglyceride (TG)-rich lipoproteins. Elevated ApoCIII levels are associated with elevated TG levels and diseases such as cardiovascular disease, metabolic syndrome, obesity and diabetes. Elevated TG levels are associated with pancreatitis. ApoCIII slows clearance of TG-rich lipoproteins by inhibiting lipolysis through inhibition of lipoprotein lipase (LPL) and through interfering with lipoprotein binding to cell-surface glycosaminoglycan matrix. Antisense compounds targeting ApoCIII have been previously disclosed in WO2004/093783 and WO2012/149495, each herein incorporated by reference in its entirety. Currently, an antisense oligonucleotide targeting ApoCIII, ISIS-APOCIII_{Rx}, is in Phase II clinical trials to assess its effectiveness in the treatment of diabetes or hypertriglyceridemia. However, there is still a need to provide patients with additional and more potent treatment options.

### Certain Conjugated Antisense Compounds Targeted to an ApoCIII Nucleic Acid

In certain embodiments, conjugated antisense compounds are targeted to an ApoCIII nucleic acid having the sequence of GENBANK® Accession No. NT_033899.8 truncated from nucleobases 20262640 to 20266603, incorporated herein as SEQ ID NO: 4. In certain such embodiments, a conjugated antisense compound is at least 90%, at least 95%, or 100% complementary to SEQ ID NO: 4. In certain embodiments, such conjugated antisense compounds comprise a conjugate comprising 1-3 GalNAc ligands. In certain embodiments, such antisense compounds comprise a conjugate disclosed herein.

In certain embodiments, conjugated antisense compounds are targeted to an ApoCIII nucleic acid having the sequence of GENBANK® Accession No. NM_000040.1, incorporated herein as SEQ ID NO: 5. In certain such embodiments, a conjugated antisense compound is at least 90%, at least 95%, or 100% complementary to SEQ ID NO: 5. In certain embodiments, such conjugated antisense compounds comprise a conjugate comprising 1-3 GalNAc ligands. In certain embodiments, such antisense compounds comprise a conjugate disclosed herein.

In certain embodiments, a conjugated antisense compound targeted to SEQ ID NO: 5 comprises an at least 8 consecutive nucleobase sequence of SEQ ID NO: 32. In certain embodiments, a conjugated antisense compound targeted to SEQ ID NO: 5 comprises a nucleobase sequence of SEQ ID NO: 32. In certain embodiments, such conjugated antisense compounds comprise a conjugate comprising 1-3 GalNAc ligands. In certain embodiments, such antisense compounds comprise a conjugate disclosed herein.

TABLE 5
Antisense Compounds targeted to ApoCIII
SEQ ID NO: 5

| | Target | Sequence | | SEQ ID |
|---|---|---|---|---|
| ISIS No | Start Site | (5′-3′) | Motif | NO |
| 304801 | 508 | AGCTTCTTGT | eeeeeddddd | 32 |
| | | CCAGCTTTAT | dddddeeeee | |
| 647535 | 508 | AGCTTCTTGT | eeeeedddddd | 32 |
| | | CCAGCTTTAT | ddddeeeeeod | |
| 616468 | 508 | AGCTTCTTGT | eeeeeddddd | 32 |
| | | CCAGCTTTAT | dddddeeeee | |
| 647536 | 508 | AGCTTCTTGT | eeoeoeoeoddddd | 32 |
| | | CCAGCTTTAT | dddddeoeoeeeod | |

### ApoCIII Therapeutic Indications

In certain embodiments, the invention provides methods for using a conjugated antisense compound targeted to an ApoCIII nucleic acid for modulating the expression of ApoCIII in a subject. In certain embodiments, the expression of ApoCIII is reduced.

In certain embodiments, the invention provides methods for using a conjugated antisense compound targeted to an ApoCIII nucleic acid in a pharmaceutical composition for treating a subject. In certain embodiments, the subject has a cardiovascular and/or metabolic disease, disorder or condition. In certain embodiments, the subject has hypertriglyceridemia, non-familial hypertriglyceridemia, familial hypertriglyceridemia, heterozygous familial hypertriglyceridemia, homozygous familial hypertriglyceridemia, mixed dyslipidemia, atherosclerosis, a risk of developing atherosclerosis, coronary heart disease, a history of coronary heart disease, early onset coronary heart disease, one or more risk factors for coronary heart disease, type II diabetes, type II diabetes with dyslipidemia, dyslipidemia, hyperlipidemia, hypercholesterolemia, hyperfattyacidemia, hepatic steatosis, non-alcoholic steatohepatitis, pancreatitis and/or non-alcoholic fatty liver disease.

In certain embodiments, the invention provides methods for using a conjugated antisense compound targeted to an ApoCIII nucleic acid in the preparation of a medicament.

### 5. C-Reactive Protein (CRP)

CRP (also known as PTX1) is an essential human acute-phase reactant produced in the liver in response to a variety of inflammatory cytokines. The protein, first identified in 1930, is highly conserved and considered to be an early indicator of infectious or inflammatory conditions. Plasma CRP levels increase 1,000-fold in response to infection, ischemia, trauma, burns, and inflammatory conditions. In clinical trials where patients receive lipid-lowering therapy, such as statin therapy, it has been demonstrated that patients having reductions in both LDL-C and CRP have a reduced risk of future coronary events relative to patients experiencing only reductions in LDL-C. Antisense compounds targeting CRP have been previously disclosed in WO2003/010284 and WO2005/005599, each herein incorporated by reference in its entirety. An antisense oligonucleotide targeting CRP, ISIS—CRP_{Rx}, is currently in Phase 2 clinical trials to study its effectiveness in treating subjects with rheumatoid arthritis and paroxysmal atrial fibrillation. However, there is still a need to provide patients with additional and more potent treatment options.

### Certain Conjugated Antisense Compounds Targeted to a CRP Nucleic Acid

In certain embodiments, conjugated antisense compounds are targeted to a CRP nucleic acid having the sequence of GENBANK® Accession No. M11725.1, incorporated herein as SEQ ID NO: 6. In certain such embodiments, a conjugated antisense compound is at least 90%, at least 95%, or 100% complementary to SEQ ID NO: 6.

In certain embodiments, a conjugated antisense compound targeted to SEQ ID NO: 6 comprises an at least 8 consecutive nucleobase sequence of SEQ ID NO: 33. In certain embodiments, a conjugated antisense compound targeted to SEQ ID NO: 6 comprises a nucleobase sequence of SEQ ID NO: 33. In certain embodiments, such conjugated antisense compounds comprise a conjugate comprising 1-3 GalNAc ligands. In certain embodiments, such antisense compounds comprise a conjugate disclosed herein.

TABLE 6
Antisense Compounds targeted to CRP SEQ ID NO: 6

| | Target | Sequence | | SEQ ID |
|---|---|---|---|---|
| ISIS No | Start Site | (5′-3′) | Motif | NO |
| 329993 | 1378 | AGCATAGTTA | eeeeeddddd | 33 |
| | | ACGAGCTCCC | dddddeeeee | |

### CRP Therapeutic Indications

In certain embodiments, the invention provides methods for using a conjugated antisense compound targeted to a CRP nucleic acid for modulating the expression of CRP in a subject. In certain embodiments, the expression of CRP is reduced.

In certain embodiments, the invention provides methods for using a conjugated antisense compound targeted to a CRP nucleic acid in a pharmaceutical composition for treating a subject. In certain embodiments, the subject has a cardiovascular and/or metabolic disease, disorder or condition. In certain embodiments, the subject has hypercholesterolemia, non-familial hypercholesterolemia, familial hypercholesterolemia, heterozygous familial hypercholesterolemia, homozygous familial hypercholesterolemia, mixed dyslipidemia, atherosclerosis, a risk of developing atherosclerosis, coronary heart disease, a history of coronary heart disease, early onset coronary heart disease, one or more risk factors for coronary heart disease. In certain embodiments, the individual has paroxysmal atrial fibrillation, acute coronary syndrome, vascular injury, arterial occlusion, unstable angina, post peripheral vascular disease, post myocardial infarction (MI), thrombosis, deep vein thrombus, end-stage renal disease (ESRD), chronic renal failure, complement activation, congestive heart failure, or systemic vasculitis. In certain embodiments, the individual has had a stroke. In certain embodiments, the individual has undergone a procedure selected from elective stent placement, angioplasty, post percutaneous transluminal angioplasty (PTCA), cardiac transplantation, renal dialysis or cardiopulmonary bypass. In certain embodiments, the individual has an inflammatory disease. In certain such embodiments, the inflammatory disease is selected from inflammatory bowel disease, ulcerative colitis, rheumatoid arthritis, or osteoarthritis.

In certain embodiments, the invention provides methods for using a conjugated antisense compound targeted to a CRP nucleic acid in the preparation of a medicament.

6. eIF4E

Overexpression of eIF4E has been reported in many human cancers and cancer-derived cell lines and also leads to oncogenic transformation of cells and invasive/metastatic phenotype in animal models. Unlike non-transformed, cultured cells, transformed cell lines express eIF4E independently of the presence of serum growth factors (Rosenwald, Cancer Lett., 1995, 98, 77-82). Excess eIF4E leads to aberrant growth and neoplastic morphology in HeLa cells and also causes tumorigenic transformation in NIH 3T₃ and Rat2 fibroblasts, as judged by anchorage-independent growth, formation of transformed foci in culture and tumor formation in nude mice (De Benedetti et al., Proc. Natl. Acad. Sci. USA, 1990, 87, 8212-8216; and Lazaris-Karatzas et al., Nature, 1990, 345, 544-547).

eIF4E is found elevated in several human cancers, including but not limited to non-Hodgkin's lymphomas, colon adenomas and carcinomas and larynx, head and neck, prostate, breast and bladder cancers (Crew et al., Br. J. Cancer, 2000, 82, 161-166; Graff et al., Clin. Exp. Metastasis, 2003, 20, 265-273; Haydon et al., Cancer, 2000, 88, 2803-2810; Kerekatte et al., Int. J. Cancer, 1995, 64, 27-31; Rosenwald et al., Oncogene, 1999, 18, 2507-2517; Wang et al., Am. J. Pathol., 1999, 155, 247-255). Upregulation of eIF4E is an early event in colon carcinogenesis, and is frequently accompanied by an increase in cyclin D1 levels (Rosenwald et al., Oncogene, 1999, 18, 2507-2517). Antisense compounds targeting eIF4E have been previously disclosed in WO2005/028628, herein incorporated by reference in its entirety. An antisense oligonucleotide targeting eIF4E, ISIS-eIF4E_{Rx}, is currently in Phase 1/2 clinical trials to study its effectiveness in treating subjects with cancer.

Certain Conjugated Antisense Compounds Targeted to an eIF4E Nucleic Acid

In certain embodiments, conjugated antisense compounds are targeted to an eIF4E nucleic acid having the sequence of GENBANK® Accession No. M15353.1, incorporated herein as SEQ ID NO: 7. In certain such embodiments, a conjugated antisense compound is at least 90%, at least 95%, or 100% complementary to SEQ ID NO: 7.

In certain embodiments, a conjugated antisense compound targeted to SEQ ID NO: 7 comprises an at least 8 consecutive nucleobase sequence of SEQ ID NO: 34. In certain embodiments, a conjugated antisense compound targeted to SEQ ID NO: 7 comprises a nucleobase sequence of SEQ ID NO: 34. In certain embodiments, such conjugated antisense compounds comprise a conjugate comprising 1-3 GalNAc ligands.

In certain embodiments, such antisense compounds comprise a conjugate disclosed herein.

TABLE 7
Antisense Compounds targeted to eIF4E SEQ ID NO: 7

| | Target | Sequence | | SEQ ID |
|---|---|---|---|---|
| ISIS No | Start Site | (5′-3′) | Motif | NO |
| 183750 | 1285 | TGTCATATTC | eeeeeddddd | 34 |
| | | CTGGATCCTT | dddddeeeee | |

eIF4E Therapeutic Indications

In certain embodiments, the invention provides methods for using a conjugated antisense compound targeted to an eIF4E nucleic acid for modulating the expression of eIF4E in a subject. In certain embodiments, the expression of eIF4E is reduced.

In certain embodiments, the invention provides methods for using a conjugated antisense compound targeted to an eIF4E nucleic acid in a pharmaceutical composition for treating a subject. In certain embodiments, the subject has cancer. In certain aspects, the cancer is prostate cancer.

In certain embodiments, the invention provides methods for using a conjugated antisense compound targeted to an eIF4E nucleic acid in the preparation of a medicament.

### 7. Factor VII

Coagulation Factor VII (also known as serum prothrombin conversion accelerator) is a key component of the tissue factor coagulation pathway. Clinicians have linked elevated levels of Factor VII activity with poor prognosis in several thrombotic diseases, such as heart attacks, and with cancer-associated thrombosis, which is the second leading cause of death in cancer patients. In preclinical studies, antisense inhibition of Factor VII rapidly reduced Factor VII activity by more than 90 percent in three days with no observed increase in bleeding, which is a common side effect of currently available anti-thrombotic drugs. Antisense compounds targeting Factor VII have been previously disclosed in WO2009/061851, WO2012/174154, and PCT Application no. PCT/US2013/025381, each herein incorporated by reference in its entirety. Clinical studies are planned to assess ISIS—FVII_{Rx} in acute clinical settings, such as following surgery, to prevent patients from developing harmful blood clots. However, there is still a need to provide patients with additional and more potent treatment options.

### Certain Conjugated Antisense Compounds Targeted to a Factor VII Nucleic Acid

In certain embodiments, conjugated antisense compounds are targeted to a Factor VII nucleic acid having the sequence of GENBANK® Accession No. NT_027140.6 truncated from nucleobases 1255000 to 1273000), incorporated herein as SEQ ID NO: 8. In certain such embodiments, a conjugated antisense compound targeted to SEQ ID NO: 8 is at least 90%, at least 95% or 10000 complementary to SEQ ID NO: 8.

In certain embodiments, a conjugated antisense compound targeted to SEQ ID NO: 8 comprises an at least 8 consecutive nucleobase sequence of SEQ ID NOs: 35-43. In certain embodiments, a conjugated antisense compound targeted to SEQ ID NO: 8 comprises a nucleobase sequence of SEQ ID NOs: 35-43. In certain embodiments, such conjugated antisense compounds comprise a conjugate comprising 1-3 GalNAc ligands. In certain embodiments, such antisense compounds comprise a conjugate disclosed herein.

TABLE 8
Antisense Compounds targeted to Factor
VII SEQ ID NO: 8

| | Target | | | SEQ |
|---|---|---|---|---|
| ISIS | Start | | | ID |
| No | Site | Sequence (5′-3′) | Motif | NO |
| 540175 | 2592 | GGACACCCACGCCCCC | eekddddddddddkke | 35 |
| | 2626 | | | |
| | 2660 | | | |
| | 2796 | | | |
| | 2966 | | | |
| | 3000 | | | |
| | 3034 | | | |
| | 3068 | | | |
| | 3153 | | | |
| | 3170 | | | |
| | 3272 | | | |
| | 3374 | | | |
| | 3578 | | | |
| | 3851 | | | |
| | 3953 | | | |
| | 4124 | | | |
| | 4260 | | | |
| | 4311 | | | |
| | 4447 | | | |
| | 4532 | | | |
| 490279 | 1387 | CCCTCCTGTGCCTGGA | eeeeedddddddddde | 36 |
| | | TGCT | eeee | |
| 473589 | 15128 | GCTAAACAACCGCCTT | kdkdkdddddddddee | 37 |
| 407935 | 15191 | ATGCATGGTGATGCTT | eeeeedddddddddde | 38 |
| | | CTGA | eeee | |
| 529804 | 15192 | CATGGTGATGCTTCTG | kddddddddddkekee | 39 |
| 534796 | 15131 | AGAGCTAAACAACCGC | Ekkddddddddddkke | 40 |
| 540162 | 2565 | ACTCCCGGGACACCCA | eekddddddddddkke | 41 |
| | 2633 | | | |
| | 2667 | | | |
| | 2735 | | | |
| | 2803 | | | |
| | 2837 | | | |
| | 2905 | | | |
| | 3007 | | | |
| | 3041 | | | |
| | 3075 | | | |
| | 3092 | | | |
| | 3279 | | | |
| | 3381 | | | |
| | 3483 | | | |
| | 3603 | | | |
| | 3722 | | | |
| | 3756 | | | |
| | 147 | | | |
| | 3858 | | | |
| | 3892 | | | |
| | 3960 | | | |
| | 4046 | | | |
| | 4131 | | | |
| | 4165 | | | |
| | 4318 | | | |
| | 4454 | | | |
| 540182 | 2692 | ACACCCTCGCCTCCGG | eekddddddddddkke | 42 |
| | 2760 | | | |
| | 2862 | | | |
| | 2930 | | | |
| | 3117 | | | |
| | 3338 | | | |
| | 3440 | | | |
| | 3508 | | | |
| | 3542 | | | |
| | 3628 | | | |
| | 3662 | | | |
| | 3781 | | | |
| | 3815 | | | |
| | 3917 | | | |
| | 4190 | | | |
| | 4224 | | | |
| | 4377 | | | |
| | 4411 | | | |
| 540191 | 3109 | GCCTCCGGAACACCCA | eekddddddddddkke | 43 |
| | 3194 | | | |
| | 3330 | | | |
| | 3432 | | | |
| | 3500 | | | |
| | 3534 | | | |
| | 3620 | | | |
| | 3654 | | | |
| | 3773 | | | |
| | 4182 | | | |
| | 4216 | | | |
| | 4369 | | | |
| | 4403 | | | |

### Factor VII Therapeutic Indications

In certain embodiments, the invention provides methods for using a conjugated antisense compound targeted to a Factor VII nucleic acid for modulating the expression of Factor VII in a subject. In certain embodiments, the expression of Factor VII is reduced.

In certain embodiments, the invention provides methods for using a conjugated antisense compound targeted to a Factor VII nucleic acid in a pharmaceutical composition for treating a subject. In certain embodiments, the subject has or is at risk of developing a thromboembolic condition, such as, heart attack, stroke, deep vein thrombosis, or pulmonary embolism. In certain embodiments, the subject is at risk of developing a thromboemoblic condition and/or otherwise in need of anticoagulant therapy. Examples of such subjects include those undergoing major orthopedic surgery and patients in need of chronic anticoagulant treatment. In certain embodiments, the subject has or is at risk of developing an inflammatory disease, disorder or condition. In certain embodiments, the subject has or is at risk of developing allergic diseases (e.g., allergic rhinitis, chronic rhinosinusitis), autoimmune diseases (e.g., multiple sclerosis, arthritis, scleroderma, psoriasis, celiac disease), cardiovascular diseases, colitis, diabetes (e.g., type 1 insulin-dependent diabetes mellitus), hypersensitivities (e.g., Type 1, 2, 3 or 4 hypersensitivity), infectious diseases (e.g., viral infection, mycobacterial infection, helminth infection), posterior uveitis, airway hyperresponsiveness, asthma, atopic dermatitis, colitis, endometriosis, thyroid disease (e.g., Graves' disease) and pancreatitis.

In certain embodiments, the invention provides methods for using a conjugated antisense compound targeted to a Factor VII nucleic acid in the preparation of a medicament.

### 8. Factor XI

Coagulation factor XI (also known as plasma thromboplastin antecedent) is an important member of the coagulation pathway. High levels of Factor XI increase the risk of thrombosis, a process involving aberrant blood clot formation responsible for most heart attacks and strokes. Elevated levels of Factor XI also increase the risk of venous thrombosis, a common problem after surgery, particularly major orthopedic procedures, such as knee or hip replacement. People who are deficient in Factor XI have a lower incidence of thromboembolic events with minimal increase in bleeding risk. Antisense compounds targeting Factor XI have been previously disclosed in WO2010/045509 and WO2010/121074, each herein incorporated by reference in its entirety. Currently, an antisense oligonucleotide targeting Factor XI, ISIS—FXI_{Rx}, is in Phase 2 clinical studies to assess the effectiveness of ISIS—FXI_{Rx} in reducing the number of thrombotic events in patients following total knee arthroplasty without increasing bleeding. However, there is still a need to provide patients with additional and more potent treatment options.

### Certain Conjugated Antisense Compounds Targeted to a Factor XI Nucleic Acid

In certain embodiments, conjugated antisense compounds are targeted to a Factor XI nucleic acid having the sequence of GENBANK® Accession No. NM_000128.3, incorporated herein as SEQ ID NO: 9. In certain such embodiments, a conjugated antisense compound is at least 90%, at least 95%, or 100% complementary to SEQ ID NO: 9.

In certain embodiments, a conjugated antisense compound targeted to SEQ ID NO: 9 comprises an at least 8 consecutive nucleobase sequence of SEQ ID NOs: 44-48. In certain embodiments, a conjugated antisense compound targeted to SEQ ID NO: 9 comprises a nucleobase sequence of SEQ ID NOs: 44-48. In certain embodiments, such conjugated antisense compounds comprise a conjugate comprising 1-3 GalNAc ligands. In certain embodiments, such antisense compounds comprise a conjugate disclosed herein.

TABLE 9
Antisense Compounds targeted to Factor
XI SEQ ID NO: 9

| | Target | | | SEQ |
|---|---|---|---|---|
| | Start | | | ID |
| ISIS No | Site | Sequence (5′-3′) | Motif | NO |
| 416858 | 1288 | ACGGCATTGGTGCACAGTTT | eeeeeddddd | 44 |
| | | | dddddeeeee | |
| 416838 | 1022 | GCAACCGGGATGATGAGTGC | eeeeeddddd | 45 |
| | | | dddddeeeee | |
| 416850 | 1278 | TGCACAGTTTCTGGCAGGCC | eeeeeddddd | 46 |
| | | | dddddeeeee | |
| 416864 | 1296 | GGCAGCGGACGGCATTGGTG | eeeeeddddd | 47 |
| | | | dddddeeeee | |
| 417002 | 1280 | GGTGCACAGTTTCTGGCAGG | eedddddddd | 48 |
| | | | dddddeeeee | |

### Factor XI Therapeutic Indications

In certain embodiments, the invention provides methods for using a conjugated antisense compound targeted to a Factor XI nucleic acid for modulating the expression of Factor XI in a subject. In certain embodiments, the expression of Factor XI is reduced.

In certain embodiments, the invention provides methods for using a conjugated antisense compound targeted to a Factor XI nucleic acid in a pharmaceutical composition for treating a subject. In certain embodiments, the subject has or is at risk of developing a thromboembolic condition, such as, heart attack, stroke, deep vein thrombosis, or pulmonary embolism. In certain embodiments, the subject is at risk of developing a thromboemoblic condition and/or otherwise in need of anticoagulant therapy. Examples of such subjects include those undergoing major orthopedic surgery and patients in need of chronic anticoagulant treatment. In certain embodiments, the subject has or is at risk of developing an inflammatory disease, disorder or condition. In certain embodiments, the subject has or is at risk of developing allergic diseases (e.g., allergic rhinitis, chronic rhinosinusitis), autoimmune diseases (e.g., multiple sclerosis, arthritis, scleroderma, psoriasis, celiac disease), cardiovascular diseases, colitis, diabetes (e.g., type 1 insulin-dependent diabetes mellitus), hypersensitivities (e.g., Type 1, 2, 3 or 4 hypersensitivity), infectious diseases (e.g., viral infection, mycobacterial infection, helminth infection), posterior uveitis, airway hyperresponsiveness, asthma, atopic dermatitis, colitis, endometriosis, thyroid disease (e.g., Graves' disease) and pancreatitis.

In certain embodiments, the invention provides methods for using a conjugated antisense compound targeted to a Factor XI nucleic acid in the preparation of a medicament.

### 9. Glucocorticoid Receptor (GCCR)

Complementary DNA clones encoding the human glucocorticoid receptor (also known as nuclear receptor subfamily 3, group C, member 1; NR3C1; GCCR; GCR; GRL; Glucocorticoid receptor, lymphocyte) were first isolated in 1985 (Hollenberg et al., Nature, 1985, 318, 635-641; Weinberger et al., Science, 1985, 228, 740-742). The gene is located on human chromosome 5q11-q13 and consists of 9 exons (Encio and Detera-Wadleigh, J Biol Chem, 1991, 266, 7182-7188; Gehring et al., Proc Natl Acad Sci USA, 1985, 82, 3751-3755).

The human glucocorticoid receptor is comprised of three major domains, the N-terminal activation domain, the central DNA-binding domain and the C-terminal ligand-binding domain (Giguere et al., Cell, 1986, 46, 645-652). In the absence of ligand, the glucocorticoid receptor forms a large heteromeric complex with several other proteins, from which it dissociates upon ligand binding.

In the liver, glucocorticoid agonists increase hepatic glucose production by activating the glucocorticoid receptor, which subsequently leads to increased expression of the gluconeogenic enzymes phosphoenolpyruvate carboxykinase (PEPCK) and glucose-6-phosphatase. Through gluconeogenesis, glucose is formed through non-hexose precursors, such as lactate, pyruvate and alanine (Link, Curr Opin Investig Drugs, 2003, 4, 421-429).

Antisense compounds targeting GCCR have been previously disclosed in WO2007/035759, WO2005/071080, and PCT application no. PCT/US2012/061984, each herein incorporated by reference in its entirety. An antisense oligonucleotide targeting GCCR, ISIS-GCCR_{Rx}, recently completed a Phase I clinical study with positive results. However, there is still a need to provide patients with additional and more potent treatment options.

### Certain Conjugated Antisense Compounds Targeted to a GCCR Nucleic Acid

In certain embodiments, conjugated antisense compounds are targeted to a GCCR nucleic acid having the sequence of the complement of GENBANK Accession No. NT_029289.10 truncated from nucleobases 3818000 to 3980000, incorporated herein as SEQ ID NO: 10. In certain such embodiments, a conjugated antisense compound targeted to SEQ ID NO: 10 is at least 90%, at least 95%, or 10000 complementary to SEQ ID NO: 10.

In certain embodiments, a conjugated antisense compound targeted to SEQ ID NO: 10 comprises an at least 8 consecutive nucleobase sequence of SEQ ID NOs: 49-59. In certain embodiments, a conjugated antisense compound targeted to SEQ ID NO: 10 comprises a nucleobase sequence of SEQ ID NOs: 49-59. In certain embodiments, such conjugated antisense compounds comprise a conjugate comprising 1-3 GalNAc ligands. In certain embodiments, such antisense compounds comprise a conjugate disclosed herein.

TABLE 10
Antisense Compounds targeted to GCCR
SEQ ID NO: 10

| | Target | | | SEQ |
|---|---|---|---|---|
| | Start | Sequence | | ID |
| ISIS No | Site | (5′-3′) | Motif | NO |
| 426115 | 65940 | GCAGCCATGGTGATCAGGAG | eeeeeddddd | 49 |
| | | | dddddeeeee | |
| 420470 | 57825 | GGTAGAAATATAGTTGTTCC | eeeeeddddd | 50 |
| | | | dddddeeeee | |
| 420476 | 59956 | TTCATGTGTCTGCATCATGT | eeeeeddddd | 51 |
| | | | dddddeeeee | |
| 426130 | 63677 | GCATCCAGCGAGCACCAAAG | eeeeeddddd | 52 |
| | | | dddddeeeee | |
| 426183 | 65938 | AGCCATGGTGATCAGGAGGC | eeeddddddd | 53 |
| | | | dddddddeee | |
| 426261 | 65938 | AGCCATGGTGATCAGGAGGC | eedddddddd | 53 |
| | | | dddddeeeee | |
| 426262 | 65939 | CAGCCATGGTGATCAGGAGG | eedddddddd | 54 |
| | | | dddddeeeee | |
| 426168 | 76224 | GTCTGGATTACAGCATAAAC | eeeeeddddd | 55 |
| | | | dddddeeeee | |
| 426246 | 76225 | GGTCTGGATTACAGCATAAA | eeeddddddd | 56 |
| | | | dddddddeee | |
| 426172 | 76229 | CCTTGGTCTGGATTACAGCA | eeeeeddddd | 57 |
| | | | dddddeeeee | |
| 426325 | 76229 | CCTTGGTCTGGATTACAGCA | eedddddddd | 58 |
| | | | dddddeeeee | |
| 426267 | 95513 | GTGCTTGTCCAGGATGATGC | eedddddddd | 59 |
| | | | dddddeeeee | |

### GCCR Therapeutic Indications

In certain embodiments, the invention provides methods for using a conjugated antisense compound targeted to a GCCR nucleic acid for modulating the expression of GCCR in a subject. In certain embodiments, the expression of GCCR is reduced.

In certain embodiments, the invention provides methods for using a conjugated antisense compound targeted to a GCCR nucleic acid in a pharmaceutical composition for treating a subject. In certain embodiments, the subject has metabolic related diseases, including metabolic syndrome, diabetes mellitus, insulin resistance, diabetic dyslipidemia, hypertriglyceridemia, obesity and weight gain.

Diabetes mellitus is characterized by numerous physical and physiological symptoms. Any symptom known to one of skill in the art to be associated with Type 2 diabetes can be ameliorated or otherwise modulated as set forth above in the methods described above. In certain embodiments, the symptom is a physical symptom selected from the group consisting of increased glucose levels, increased weight gain, frequent urination, unusual thirst, extreme hunger, extreme fatigue, blurred vision, frequent infections, tingling or numbness at the extremities, dry and itchy skin, weight loss, slow-healing sores, and swollen gums. In certain embodiments, the symptom is a physiological symptom selected from the group consisting of increased insulin resistance, increased glucose levels, increased fat mass, decreased metabolic rate, decreased glucose clearance, decreased glucose tolerance, decreased insulin sensitivity, decreased hepatic insulin sensitivity, increased adipose tissue size and weight, increased body fat, and increased body weight.

In certain embodiments, the invention provides methods for using a conjugated antisense compound targeted to a GCCR nucleic acid in the preparation of a medicament.

### 10. Glucagon Receptor (GCGR)

Diabetes is a chronic metabolic disorder characterized by impaired insulin secretion and/or action. In type 2 diabetes (T2DM), insulin resistance leads to an inability of insulin to control the activity of gluconeogenic enzymes, and many subjects also exhibit inappropriate levels of circulating glucagon in the fasting and postprandial state. Glucagon is secreted from the α-cells of the pancreatic islets and regulates glucose homeostasis through modulation of hepatic glucose production (Quesada et al., J. Endocrinol. 2008. 199: 5-19). Glucagon exerts its action on target tissues via the activation of its receptor, GCGR. The glucagon receptor is a 62 kDa protein that is a member of the class B G-protein coupled family of receptors (Brubaker et al., Recept. Channels. 2002. 8: 179-88). GCGR activation leads to signal transduction by G proteins (Gₛα and G_{q}), whereby Gₛα activates adenylate cyclase, which causes cAMP production, resulting in an increase in levels of protein kinase A. GCGR signaling in the liver results in increased hepatic glucose production by induction of glycogenolysis and gluconeogenesis along with inhibition of glycogenesis (Jiang and Zhang. Am. J. Physiol. Endocrinol. Metab. 2003. 284: E671-E678). GCGR is also expressed in extrahepatic tissues, which includes heart, intestinal smooth muscle, kidney, brain, and adipose tissue (Hansen et al., Peptides. 1995. 16: 1163-1166).

Antisense compounds targeting GCGR have been previously disclosed in WO2004/096996, WO2004/096016, WO2007/035771, and WO2013/043817, each herein incorporated by reference in its entirety. An antisense oligonucleotide targeting GCGR, ISIS-GCGR_{Rx}, recently completed a Phase I clinical study with positive results. However, there is still a need to provide patients with additional and more potent treatment options.

### Certain Conjugated Antisense Compounds Targeted to a GCGR Nucleic Acid

In certain embodiments, conjugated antisense compounds are targeted to a GCGR nucleic acid having the sequence of GENBANK® Accession No NW_926918.1 truncated from nucleobases 16865000 to 16885000, incorporated herein as SEQ ID NO: 11. In certain such embodiments, a conjugated antisense compound targeted to SEQ ID NO: 11 is at least 90%, at least 95%, or 10000 complementary to SEQ ID NO: 11.

In certain embodiments, a conjugated antisense compound targeted to SEQ ID NO: 11 comprises an at least 8 consecutive nucleobase sequence of SEQ ID NOs: 60-67. In certain embodiments, a conjugated antisense compound targeted to SEQ ID NO: 11 comprises a nucleobase sequence of SEQ ID NOs: 60-67. In certain embodiments, such conjugated antisense compounds comprise a conjugate comprising 1-3 GalNAc ligands. In certain embodiments, such antisense compounds comprise a conjugate disclosed herein.

TABLE 11
Antisense Compounds targeted to GCGR
SEQ ID NO: 11

| | Target | | | SEQ |
|---|---|---|---|---|
| | Start | Sequence | | ID |
| ISIS No | Site | (5′-3′) | Motif | NO |
| 449884 | 7270 | GGTTCCCGA | eeedddddddddd | 60 |
| | 7295 | GGTGCCCA | eeee | |
| | 7319 | | | |
| | 7344 | | | |
| | 7368 | | | |
| | 7392 | | | |
| | 7416 | | | |
| | 7440 | | | |
| 398471 | 8133 | TCCACAGGCC | eeeeedddddddddd | 61 |
| | | ACAGGTGGGC | eeeee | |
| 436140 | 15743 | CTCTTTATTG | eeeeedddddddddd | 62 |
| | | TTGGAGGACA | eeeee | |
| 448766 | 9804 | GCAAGGCTCG | eeeeedddddddddd | 63 |
| | | GTTGGGCTTC | eeeee | |
| 459014 | 10718 | GGGCAATGCA | eeedddddddddd | 64 |
| | | GTCCTGG | eeee | |
| 459032 | 7783 | GAAGGTGACAC | eeedddddddddd | 65 |
| | | CAGCCT | eeee | |
| 459040 | 8144 | GCTCAGCATCC | eeedddddddddd | 66 |
| | | ACAGGC | eeee | |
| 459157 | 7267 | GGGTTCCCGAG | eeeeedddddddddd | 67 |
| | 7292 | GTGCCCAATG | eeeeee | |
| | 7316 | | | |
| | 7341 | | | |
| | 7365 | | | |
| | 7389 | | | |
| | 7437 | | | |

### GCGR Therapeutic Indications

In certain embodiments, the invention provides methods for using a conjugated antisense compound targeted to a GCGR nucleic acid for modulating the expression of GCGR in a subject. In certain embodiments, the expression of GCGR is reduced.

In certain embodiments, the invention provides methods for using a conjugated antisense compound targeted to a GCGR nucleic acid in a pharmaceutical composition for treating a subject. In certain embodiments, the subject has metabolic related diseases, including metabolic syndrome, diabetes mellitus, insulin resistance, diabetic dyslipidemia, hypertriglyceridemia, obesity and weight gain.

Diabetes mellitus is characterized by numerous physical and physiological signs and/or symptoms. Any symptom known to one of skill in the art to be associated with Type 2 diabetes can be ameliorated or otherwise modulated as set forth above in the methods described above. In certain embodiments, the symptom or sign is a physical symptom or sign such as increased glucose levels, increased weight gain, frequent urination, unusual thirst, extreme hunger, extreme fatigue, blurred vision, frequent infections, tingling or numbness at the extremities, dry and itchy skin, weight loss, slow-healing sores, and swollen gums. In certain embodiments, the symptom or sign is a physiological symptom or sign selected from the group consisting of increased insulin resistance, increased glucose levels, increased fat mass, decreased metabolic rate, decreased glucose clearance, decreased glucose tolerance, decreased insulin sensitivity, decreased hepatic insulin sensitivity, increased adipose tissue size and weight, increased body fat, and increased body weight.

In certain embodiments, the invention provides methods for using a conjugated antisense compound targeted to a GCGR nucleic acid in the preparation of a medicament.

### 11. Hepatitis B (HBV)

Hepatitis B is a viral disease transmitted parenterally by contaminated material such as blood and blood products, contaminated needles, sexually and vertically from infected or carrier mothers to their offspring. It is estimated by the World Health Organization that more than 2 billion people have been infected worldwide, with about 4 million acute cases per year, 1 million deaths per year, and 350-400 million chronic carriers (World Health Organization: Geographic Prevalence of Hepatitis B Prevalence, 2004. who.int/vaccines-surveillance/graphics/htmls/hepbprev.htm).

The virus, HBV, is a double-stranded hepatotropic virus which infects only humans and non-human primates. Viral replication takes place predominantly in the liver and, to a lesser extent, in the kidneys, pancreas, bone marrow and spleen (Hepatitis B virus biology. Microbiol Mol Biol Rev. 64: 2000; 51-68.). Viral and immune markers are detectable in blood and characteristic antigen-antibody patterns evolve over time. The first detectable viral marker is HBsAg, followed by hepatitis B e antigen (HBeAg) and HBV DNA. Titers may be high during the incubation period, but HBV DNA and HBeAg levels begin to fall at the onset of illness and may be undetectable at the time of peak clinical illness (Hepatitis B virus infection—natural history and clinical consequences. N Engl J Med. 350: 2004; 1118-1129). HBeAg is a viral marker detectable in blood and correlates with active viral replication, and therefore high viral load and infectivity (Hepatitis B e antigen—the dangerous end game of hepatitis B. N Engl J Med. 347: 2002; 208-210). The presence of anti-HBsAb and anti-HBcAb (IgG) indicates recovery and immunity in a previously infected individual.

Currently the recommended therapies for chronic HBV infection by the American Association for the Study of Liver Diseases (AASLD) and the European Association for the Study of the Liver (EASL) include interferon alpha (INFα), pegylated interferon alpha-2a (Peg-IFN2a), entecavir, and tenofovir. The nucleoside and nucleobase therapies, entecavir and tenofovir, are successful at reducing viral load, but the rates of HBeAg seroconversion and HBsAg loss are even lower than those obtained using IFNα therapy. Other similar therapies, including lamivudine (3TC), telbivudine (LdT), and adefovir are also used, but for nucleoside/nucleobase therapies in general, the emergence of resistance limits therapeutic efficacy.

Thus, there is a need in the art to discover and develop new anti-viral therapies. Additionally, there is a need for new anti-HBV therapies capable of increasing HBeAg and HBsAg seroconversion rates. Recent clinical research has found a correlation between seroconversion and reductions in HBeAg (Fried et al (2008) Hepatology 47:428) and reductions in HBsAg (Moucari et al (2009) Hepatology 49:1151). Reductions in antigen levels may have allowed immunological control of HBV infection because high levels of antigens are thought to induce immunological tolerance. Current nucleoside therapies for HBV are capable of dramatic reductions in serum levels of HBV but have little impact on HBeAg and HBsAg levels.

Antisense compounds targeting HBV have been previously disclosed in WO2011/047312, WO2012/145674, and WO2012/145697, each herein incorporated by reference in its entirety. Clinical studies are planned to assess the effect of antisense compounds targeting HBV in patients. However, there is still a need to provide patients with additional and more potent treatment options.

### Certain Conjugated Antisense Compounds Targeted to a HBV Nucleic Acid

In certain embodiments, conjugated antisense compounds are targeted to a HBV nucleic acid having the sequence of GENBANK® Accession No. U95551.1, incorporated herein as SEQ ID NO: 12. In certain such embodiments, a conjugated antisense compound targeted to SEQ ID NO: 12 is at least 90%, at least 95%, or 100% complementary to SEQ ID NO: 12.

In certain embodiments, a conjugated antisense compound targeted to SEQ ID NO: 12 comprises an at least 8 consecutive nucleobase sequence of SEQ ID NO: 68. In certain embodiments, a conjugated antisense compound targeted to SEQ ID NO: 12 comprises a nucleobase sequence of SEQ ID NO: 68. In certain embodiments, such conjugated antisense compounds comprise a conjugate comprising 1-3 GalNAc ligands. In certain embodiments, such antisense compounds comprise a conjugate disclosed herein.

TABLE 12
Antisense Compounds targeted to HBV
SEQ ID NO: 12

| | Target | | | SEQ |
|---|---|---|---|---|
| | Start | Sequence | | ID |
| ISIS No | Site | (5′-3′) | Motif | NO |
| 505358 | 1583 | GCAGAGGTGA | eeeeedddddddddd | 68 |
| | | AGCGAAGTGC | eeeee | |

### HBV Therapeutic Indications

In certain embodiments, the invention provides methods for using a conjugated antisense compound targeted to a HBV nucleic acid for modulating the expression of HBV in a subject. In certain embodiments, the expression of HBV is reduced.

In certain embodiments, the invention provides methods for using a conjugated antisense compound targeted to a HBV nucleic acid in a pharmaceutical composition for treating a subject. In certain embodiments, the subject has a HBV-related condition. In certain embodiments, the HBV-related condition includes, but is not limited to, chronic HBV infection, inflammation, fibrosis, cirrhosis, liver cancer, serum hepatitis, jaundice, liver cancer, liver inflammation, liver fibrosis, liver cirrhosis, liver failure, diffuse hepatocellular inflammatory disease, hemophagocytic syndrome, serum hepatitis, and HBV viremia. In certain embodiments, the HBV-related condition may have which may include any or all of the following: flu-like illness, weakness, aches, headache, fever, loss of appetite, diarrhea, jaundice, nausea and vomiting, pain over the liver area of the body, clay- or grey-colored stool, itching all over, and dark-colored urine, when coupled with a positive test for presence of a hepatitis B virus, a hepatitis B viral antigen, or a positive test for the presence of an antibody specific for a hepatitis B viral antigen. In certain embodiments, the subject is at risk for an HBV-related condition. This includes subjects having one or more risk factors for developing an HBV-related condition, including sexual exposure to an individual infected with Hepatitis B virus, living in the same house as an individual with a lifelong hepatitis B virus infection, exposure to human blood infected with the hepatitis B virus, injection of illicit drugs, being a person who has hemophilia, and visiting an area where hepatitis B is common. In certain embodiments, the subject has been identified as in need of treatment for an HBV-related condition.

In certain embodiments, the invention provides methods for using a conjugated antisense compound targeted to a HBV nucleic acid in the preparation of a medicament.

### 12. Protein Tyrosine Phosphatase 1B (PTP1B)

PTP1B is a member of a family of PTPs (Barford, et al., Science 1994. 263: 1397-1404) and is a cytosolic enzyme (Neel and Tonks, Curr. Opin. Cell Biol. 1997. 9: 193-204). PTP1B is expressed ubiquitously including tissues that are key regulators of insulin metabolism such as liver, muscle and fat (Goldstein, Receptor 1993. 3: 1-15), where it is the main PTP enzyme.

PTP1B is considered to be a negative regulator of insulin signaling. PTP1B interacts with and dephosphorylates the insulin receptor, thus attenuating and potentially terminating the insulin signalling transduction (Goldstein et al., J. Biol. Chem. 2000. 275: 4383-4389). The physiological role of PTP1B in insulin signalling has been demonstrated in knockout mice models. Mice lacking the PTP1B gene were protected against insulin resistance and obesity (Elchebly et al., Science 1999. 283: 1544-1548). PTP1B-deficient mice had low adiposity, increased basal metabolic rate as well as total energy expenditure and were protected from diet-induced obesity. Insulin-stimulated glucose uptake was elevated in skeletal muscle, whereas adipose tissue was unaffected providing evidence that increased insulin sensitivity in PTP1B-deficient mice was tissue-specific (Klaman et al., Mol. Cell. Biol. 2000. 20: 5479-5489). These mice were phenotypically normal and were also resistant to diet-induced obesity, insulin resistance and had significantly lower triglyceride levels on a high-fat diet. Therefore, inhibition of PTP1B in patients suffering from Type II diabetes, metabolic syndrome, diabetic dyslipidemia, or related metabolic diseases would be beneficial.

Antisense compounds targeting PTP1B have been previously disclosed in WO2001/053528, WO2002/092772, WO2004/071407, WO2006/044531, WO2012/142458, WO2006/044531, and WO2012/142458, each herein incorporated by reference in its entirety. An antisense oligonucleotide targeting PTP1B, ISIS-PTP1B_{Rx}, recently completed a Phase I clinical study with positive results. However, there is still a need to provide patients with additional and more potent treatment options.

### Certain Conjugated Antisense Compounds Targeted to a PTP1B Nucleic Acid

In certain embodiments, conjugated antisense compounds are targeted to a PTP1B nucleic acid having the sequence of GENBANK® Accession No. NM_002827.2, incorporated herein as SEQ ID NO: 13 or GENBANK Accession NT_011362.9 truncated from nucleobases 14178000 to 14256000, incorporated herein as SEQ ID NO: 14. In certain such embodiments, a conjugated antisense compound targeted to SEQ ID NO: 13 is at least 90%, at least 95%, or 100% complementary to SEQ ID NO: 13.

In certain embodiments, a conjugated antisense compound targeted to SEQ ID NO: 13 comprises an at least 8 consecutive nucleobase sequence of SEQ ID NOs: 69-72. In certain embodiments, a conjugated antisense compound targeted to SEQ ID NO: 13 comprises a nucleobase sequence of SEQ ID NOs: 69-72. In certain embodiments, such conjugated antisense compounds comprise a conjugate comprising 1-3 GalNAc ligands. In certain embodiments, such antisense compounds comprise a conjugate disclosed herein.

TABLE 13
Conjugated Antisense Compounds targeted to
PTP1B SEQ ID NO: 13

| | Target | | | |
|---|---|---|---|---|
| | Start | | | SEQ |
| | Site | Sequence | | ID |
| ISIS No | on mRNA | (5′-3′) | Chemistry | NO |
| 404173 | 3290 | AATGGTTTAT | eeeeedddddddddd | 69 |
| | | TCCATGGCCA | eeeee | |
| 409826 | 3287 | GGTTTATTCC | eeeeedddddddddd | 70 |
| | | ATGGCCATTG | eeeee | |
| 142082 | 3291 | AAATGGTTTA | eeeeedddddddddd | 71 |
| | | TTCCATGGCC | eeeee | |
| 446431 | 3292 | AATGGTTTAT | eeeedddddddddde | 72 |
| | | TCCATGGC | eee | |

### PTP1B Therapeutic Indications

In certain embodiments, the invention provides methods for using a conjugated antisense compound targeted to a PTP1B nucleic acid for modulating the expression of PTP1B in a subject. In certain embodiments, the expression of PTP1B is reduced.

In certain embodiments, the invention provides methods for using a conjugated antisense compound targeted to a PTP1B nucleic acid in a pharmaceutical composition for treating a subject. In certain embodiments, the subject has metabolic related diseases, including metabolic syndrome, diabetes mellitus, insulin resistance, diabetic dyslipidemia, hypertriglyceridemia, obesity and weight gain.

Diabetes mellitus is characterized by numerous physical and physiological symptoms. Any symptom known to one of skill in the art to be associated with Type 2 diabetes can be ameliorated or otherwise modulated as set forth above in the methods described above. In certain embodiments, the symptom is a physical symptom selected from the group consisting of increased glucose levels, increased weight gain, frequent urination, unusual thirst, extreme hunger, extreme fatigue, blurred vision, frequent infections, tingling or numbness at the extremities, dry and itchy skin, weight loss, slow-healing sores, and swollen gums. In certain embodiments, the symptom is a physiological symptom selected from the group consisting of increased insulin resistance, increased fat mass, decreased metabolic rate, decreased glucose clearance, decreased glucose tolerance, decreased insulin sensitivity, decreased hepatic insulin sensitivity, increased adipose tissue size and weight, increased body fat, and increased body weight.

In certain embodiments, the invention provides methods for using a conjugated antisense compound targeted to a PTP1B nucleic acid in the preparation of a medicament.

### 13. STAT3

The STAT (signal transducers and activators of transcription) family of proteins comprises DNA-binding proteins that play a dual role in signal transduction and activation of transcription. Presently, there are six distinct members of the STAT family (STAT1, STAT2, STAT3, STAT4, STAT5, and STAT6) and several isoforms (STAT1α, STAT1β, STAT3 α and STAT3β). The activities of the STATs are modulated by various cytokines and mitogenic stimuli. Binding of a cytokine to its receptor results in the activation of Janus protein tyrosine kinases (JAKs) associated with these receptors. This phosphorylates STAT, resulting in translocation to the nucleus and transcriptional activation of STAT responsive genes. Phosphorylation on a specific tyrosine residue on the STATs results in their activation, resulting in the formation of homodimers and/or heterodimers of STAT which bind to specific gene promoter sequences. Events mediated by cytokines through STAT activation include cell proliferation and differentiation and prevention of apoptosis.

The specificity of STAT activation is due to specific cytokines, i.e., each STAT is responsive to a small number of specific cytokines. Other non-cytokine signaling molecules, such as growth factors, have also been found to activate STATs. Binding of these factors to a cell surface receptor associated with protein tyrosine kinase also results in phosphorylation of STAT.

STAT3 (also acute phase response factor (APRF)), in particular, has been found to be responsive to interleukin-6 (IL-6) as well as epidermal growth factor (EGF) (Darnell, Jr., J. E., et al., Science, 1994, 264, 1415-1421). In addition, STAT3 has been found to have an important role in signal transduction by interferons (Yang, C.-H., et al., Proc. Natl. Acad. Sci. USA, 1998, 95, 5568-5572). Evidence exists suggesting that STAT3 may be regulated by the MAPK pathway. ERK2 induces serine phosphorylation and also associates with STAT3 (Jain, N., et al., Oncogene, 1998, 17, 3157-3167).

STAT3 is expressed in most cell types (Zhong, Z., et al., Proc. Natl. Acad. Sci. USA, 1994, 91, 4806-4810). It induces the expression of genes involved in response to tissue injury and inflammation. STAT3 has also been shown to prevent apoptosis through the expression of bcl-2 (Fukada, T., et al., Immunity, 1996, 5, 449-460).

Recently, STAT3 was detected in the mitochondria of transformed cells, and was shown to facilitate glycolytic and oxidative phosphorylation activities similar to that of cancer cells (Gough, D. J., et al., Science, 2009, 324, 1713-1716). The inhibition of STAT3 in the mitochondria impaired malignant transformation by activated Ras. The data confirms a Ras-mediated transformation function for STAT3 in the mitochondria in addition to its nuclear roles.

Aberrant expression of or constitutive expression of STAT3 is associated with a number of disease processes.

Antisense compounds targeting STAT3 have been previously disclosed in WO2012/135736 and WO2005/083124, each herein incorporated by reference in its entirety. An antisense oligonucleotide targeting STAT3, ISIS-STAT3_{Rx}, is currently in Phase 1/2 clinical trials to study its effectiveness in treating subjects with cancer. However, there is still a need to provide patients with additional and more potent treatment options.

### Certain Conjugated Antisense Compounds Targeted to a STAT3 Nucleic Acid

In certain embodiments, conjugated antisense compounds are targeted to a STAT3 nucleic acid having the sequence of GENBANK® Accession No. NM_139276.2, incorporated herein as SEQ ID NO: 15. In certain such embodiments, a conjugated antisense compound targeted to SEQ ID NO: 15 is at least 90%, at least 95%, or 100% complementary to SEQ ID NO: 15.

In certain embodiments, a conjugated antisense compound targeted to SEQ ID NO: 15 comprises an at least 8 consecutive nucleobase sequence of SEQ ID NO: 73. In certain embodiments, a conjugated antisense compound targeted to SEQ ID NO: 15 comprises a nucleobase sequence of SEQ ID NO: 73. In certain embodiments, such conjugated antisense compounds comprise a conjugate comprising 1-3 GalNAc ligands. In certain embodiments, such antisense compounds comprise a conjugate disclosed herein.

TABLE 14
Antisense Compounds targeted to STAT3
SEQ ID NO: 15

| | Target | | | SEQ |
|---|---|---|---|---|
| | Start | Sequence | | ID |
| ISIS No | Site | (5′-3′) | Motif | NO |
| 481464 | 3016 | CTATTTGG | kkkddddddddddkkk | 73 |
| | | ATGTCAGC | | |

### STAT3 Therapeutic Indications

In certain embodiments, the invention provides methods for using a conjugated antisense compound targeted to a STAT3 nucleic acid for modulating the expression of STAT3 in a subject. In certain embodiments, the expression of STAT3 is reduced.

In certain embodiments, the invention provides methods for using a conjugated antisense compound targeted to a STAT3 nucleic acid in a pharmaceutical composition for treating a subject. In certain embodiments, the subject has a hyperproliferative disease, disorder or condition. In certain embodiments such hyperproliferative disease, disorder, and condition include cancer as well as associated malignancies and metastases. In certain embodiments, such cancers include lung cancer, including non small cell lung cancer (NSCLC), pancreatic cancer, colorectal cancer, multiple myeloma, hepatocellular carcinoma (HCC), glioblastoma, ovarian cancer, osteosarcoma, head and neck cancer, breast cancer, epidermoid carcinomas, intestinal adenomas, prostate cancer, and gastric cancer.

In certain embodiments, the invention provides methods for using a conjugated antisense compound targeted to a STAT3 nucleic acid in the preparation of a medicament.

### 14. Transthyretin (TTR)

TTR (also known as prealbumin, hyperthytoxinemia, dysprealbuminemic, thyroxine; senile systemic amyloidosis, amyloid polyneuropathy, amyloidosis I, PALB; dystransthyretinemic, HST2651; TBPA; dysprealbuminemic euthyroidal hyperthyroxinemia) is a serum/plasma and cerebrospinal fluid protein responsible for the transport of thyroxine and retinol (Sakaki et al, Mol Biol Med. 1989, 6:161-8). Structurally, TTR is a homotetramer; point mutations and misfolding of the protein leads to deposition of amyloid fibrils and is associated with disorders, such as senile systemic amyloidosis (SSA), familial amyloid polyneuropathy (FAP), and familial amyloid cardiopathy (FAC).

TTR is synthesized primarily by the liver and the choroid plexus of the brain and, to a lesser degree, by the retina in humans (Palha, Clin Chem Lab Med, 2002, 40, 1292-1300). Transthyretin that is synthesized in the liver is secreted into the blood, whereas transthyretin originating in the choroid plexus is destined for the CSF. In the choroid plexus, transthyretin synthesis represents about 20% of total local protein synthesis and as much as 25% of the total CSF protein (Dickson et al., J Biol Chem, 1986, 261, 3475-3478).

With the availability of genetic and immunohistochemical diagnostic tests, patients with TTR amyloidosis have been found in many nations worldwide. Recent studies indicate that TTR amyloidosis is not a rare endemic disease as previously thought, and may affect as much as 25% of the elderly population (Tanskanen et al, Ann Med. 2008; 40(3):232-9).

At the biochemical level, TTR was identified as the major protein component in the amyloid deposits of FAP patients (Costa et al, Proc. Natl. Acad. Sci. USA 1978, 75:4499-4503) and later, a substitution of methionine for valine at position 30 of the protein was found to be the most common molecular defect causing the disease (Saraiva et al, J Clin. Invest. 1984, 74: 104-119). In FAP, widespread systemic extracellular deposition of TTR aggregates and amyloid fibrils occurs throughout the connective tissue, particularly in the peripheral nervous system (Sousa and Saraiva, Prog. Neurobiol. 2003, 71: 385-400). Following TTR deposition, axonal degeneration occurs, starting in the unmyelinated and myelinated fibers of low diameter, and ultimately leading to neuronal loss at ganglionic sites.

Antisense compounds targeting TTR have been previously disclosed in US2005/0244869, WO2010/017509, and WO2011/139917, each herein incorporated by reference in its entirety. An antisense oligonucleotide targeting TTR, ISIS-TTR_{Rx}, is currently in Phase 2/3 clinical trials to study its effectiveness in treating subjects with Familial Amyloid Polyneuropathy. However, there is still a need to provide patients with additional and more potent treatment options.

### Certain Conjugated Antisense Compounds Targeted to a TTR Nucleic Acid

In certain embodiments, conjugated antisense compounds are targeted to a TTR nucleic acid having the sequence of GENBANK® Accession No. NM_000371.3, incorporated herein as SEQ ID NO: 16. In certain such embodiments, a conjugated antisense compound targeted to SEQ ID NO: 16 is at least 90%, at least 95%, or 100% complementary to SEQ ID NO: 16.

In certain embodiments, a conjugated antisense compound targeted to SEQ ID NO: 16 comprises an at least 8 consecutive nucleobase sequence of SEQ ID NOs: 74-81. In certain embodiments, a conjugated antisense compound targeted to SEQ ID NO: 16 comprises a nucleobase sequence of SEQ ID NO: 74-81. In certain embodiments, such conjugated antisense compounds comprise a conjugate comprising 1-3 GalNAc ligands. In certain embodiments, such antisense compounds comprise a conjugate disclosed herein.

TABLE 15
Antisense Compounds targeted to TTR
SEQ ID NO: 16

| | Target | | | SEQ |
|---|---|---|---|---|
| ISIS | Start | Sequence | | ID |
| No | Site | (5′-3′) | Motif | NO |
| 420915 | 508 | TCTTGGTTAC | eeeeeddddddddddeeeee | 74 |
| | | ATGAAATCCC | | |
| 304299 | 507 | CTTGGTTACA | eeeeeddddddddddeeeee | 75 |
| | | TGAAATCCCA | | |
| 420921 | 515 | GGAATACTCT | eeeeeddddddddddeeeee | 76 |
| | | TGGTTACATG | | |
| 420922 | 516 | TGGAATACTC | eeeeeddddddddddeeeee | 77 |
| | | TTGGTTACAT | | |
| 420950 | 580 | TTTTATTGTC | eeeeeddddddddddeeeee | 78 |
| | | TCTGCCTGGA | | |
| 420955 | 585 | GAATGTTTTA | eeeeeddddddddddeeeee | 79 |
| | | TTGTCTCTGC | | |
| 420957 | 587 | AGGAATGTTT | eeeeeddddddddddeeeee | 80 |
| | | TATTGTCTCT | | |
| 420959 | 589 | ACAGGAATGT | eeeeeddddddddddeeeee | 81 |
| | | TTTATTGTCT | | |

### TTR Therapeutic Indications

In certain embodiments, the invention provides methods for using a conjugated antisense compound targeted to a TTR nucleic acid for modulating the expression of TTR in a subject. In certain embodiments, the expression of TTR is reduced.

In certain embodiments, the invention provides methods for using a conjugated antisense compound targeted to a TTR nucleic acid in a pharmaceutical composition for treating a subject. In certain embodiments, the subject has a transthyretin related disease, disorder or condition, or symptom thereof. In certain embodiments, the transthyretin related disease, disorder or condition is transthyretin amyloidosis. “Transthyretin-related amyloidosis” or “transthyretin amyloidosis” or “Transthyretin amyloid disease”, as used herein, is any pathology or disease associated with dysfunction or dysregulation of transthyretin that result in formation of transthyretin-containing amyloid fibrils. Transthyretin amyloidosis includes, but is not limited to, hereditary TTR amyloidosis, leptomeningeal amyloidosis, familial amyloid polyneuropathy (FAP), familial amyloid cardiomyopathy, familial oculoleptomeningeal amyloidosis, senile cardiac amyloidosis, or senile systemic amyloidosis.

In certain embodiments, the invention provides methods for using a conjugated antisense compound targeted to a TTR nucleic acid in the preparation of a medicament.

### 15. PCSK9

PCSK9 (also known as Proprotein convertase subtilisin kexin 9) is a member of the subtilisin serine protease family. The other eight mammalian subtilisin proteases, PCSK1-PCSK8 (also called PC1/3, PC2, furin, PC4, PC5/6, PACE4, PC7, and S1P/SKI-1) are proprotein convertases that process a wide variety of proteins in the secretory pathway and play roles in diverse biological processes (Bergeron, F. (2000) J. Mol. Endocrinol. 24, 1-22, Gensberg, K., (1998) Semin. Cell Dev. Biol. 9, 11-17, Seidah, N. G. (1999) Brain Res. 848, 45-62, Taylor, N. A., (2003) FASEB J. 17, 1215-1227, and Zhou, A., (1999) J. Biol. Chem. 274, 20745-20748). PCSK9 has been proposed to play a role in cholesterol metabolism. PCSK9 mRNA expression is down-regulated by dietary cholesterol feeding in mice (Maxwell, K. N., (2003) J. Lipid Res. 44, 2109-2119), up-regulated by statins in HepG2 cells (Dubuc, G., (2004) Arterioscler. Thromb. Vasc. Biol. 24, 1454-1459), and up-regulated in sterol regulatory element binding protein (SREBP) transgenic mice (Horton, J. D., (2003) Proc. Natl. Acad. Sci. USA 100, 12027-12032), similar to the cholesterol biosynthetic enzymes and the low-density lipoprotein receptor (LDLR). Furthermore, PCSK9 missense mutations have been found to be associated with a form of autosomal dominant hypercholesterolemia (Hchola3) (Abifadel, M., et al. (2003) Nat. Genet. 34, 154-156, Timms, K. M., (2004) Hum. Genet. 114, 349-353, Leren, T. P. (2004) Clin. Genet. 65, 419-422). PCSK9 may also play a role in determining LDL cholesterol levels in the general population, because single-nucleotide polymorphisms (SNPs) have been associated with cholesterol levels in a Japanese population (Shioji, K., (2004) J. Hum. Genet. 49, 109-114).

Antisense compounds targeting PCSK9 have been previously disclosed in U.S. Pat. Nos. 8,084,437; 8,093,222; 8,664,190; and International applications WO 2008/066776 and WO 2009/148605. However, there is still a need to provide patients with additional and more potent treatment options.

### Certain Conjugated Antisense Compounds Targeted to a PCSK9 Nucleic Acid

In certain embodiments, conjugated antisense compounds are targeted to a PCSK9 nucleic acid having the sequence of GENBANK® Accession NM_174936.3, incorporated herein as SEQ ID NO: 82. In certain such embodiments, a conjugated antisense compound targeted to SEQ ID NO: 82 is at least 90%, at least 95%, or 100% complementary to SEQ ID NO: 82.

In certain embodiments, a conjugated antisense compound targeted to SEQ ID NO: 82 comprises an at least 8 consecutive nucleobase sequence of SEQ ID NOs: 83-86. In certain embodiments, a conjugated antisense compound targeted to SEQ ID NO: 82 comprises a nucleobase sequence of SEQ ID NO: 83-86. In certain embodiments, such conjugated antisense compounds comprise a conjugate comprising 1-3 GalNAc ligands. In certain embodiments, such antisense compounds comprise a conjugate disclosed herein.

TABLE 16
Antisense Compounds targeted to PCSK9
SEQ ID NO: 156

| | Target | | | SEQ |
|---|---|---|---|---|
| ISIS | Start | Sequence | | ID |
| No | Site | (5′-3′) | Motif | NO |
| 405879 | 1073 | CCTTGGCCAC | eeeeeddddddddddeeeee | 83 |
| | | GCCGGCATCC | | |
| 431131 | 1015 | GTCACACTTG | eeeeeddddddddddeeeee | 84 |
| | | CTGGCCTGTC | | |
| 405995 | 2001 | TGGCAGTGGA | eeeeeddddddddddeeeee | 85 |
| | | CACGGGTCCC | | |
| 480604 | 3381 | ACTCACCGAG | eeeeeddddddddddeeeee | 86 |
| | | CTTCCTGGTC | | |

### PCSK9 Therapeutic Indications

In certain embodiments, the invention provides methods for using a conjugated antisense compound targeted to a PCSK9 nucleic acid for modulating the expression of PCSK9 in a subject. In certain embodiments, the expression of PCSK9 is reduced.

In certain embodiments, the invention provides methods for using a conjugated antisense compound targeted to a PCSK9 nucleic acid in a pharmaceutical composition for treating a subject. In certain embodiments, the subject has a PCSK9 related disease, disorder or condition, or symptom thereof. In certain embodiments, the PCSK9 related disease, disorder or condition is a metabolic or cardiovascular disease.

In certain embodiments, the invention provides methods for using a conjugated antisense compound targeted to a PCSK9 nucleic acid in the preparation of a medicament.

### 16. Complement Factor B

The complement system is part of the host innate immune system involved in lysing foreign cells, enhancing phagocytosis of antigens, clumping antigen-bearing agents, and attracting macrophages and neutrophils. The complement system is divided into three initiation pathways—the classical, lectin, and alternative pathways—that converge at component C3 to generate an enzyme complex known as C3 convertase, which cleaves C3 into C3a and C3b. C3b associates with C3 convertase mediated by CFB and results in generation of C5 convertase, which cleaves C5 into C5a and C5b, which initiates the membrane attack pathway resulting in the formation of the membrane attack complex (MAC) comprising components C5b, C6, C7, C8, and C9. The membrane-attack complex (MAC) forms transmembrane channels and disrupts the phospholipid bilayer of target cells, leading to cell lysis.

In the homeostatic state, the alternative pathway is continuously activated at a low “tickover” level as a result of activation of the alternative pathway by spontaneous hydrolysis of C3 and the production of C3b, which generates C5 convertase.

### Oligonucleotide Designed to Target Human Complement Factor B (CFB)

TABLE 17

| | | SEQ |
|---|---|---|
| Isis | | ID |
| No. | Sequence (5′ to 3′) | No. |
| 588540 | AₑₛTₑₛ^{m}Cₑₛ^{m}Cₑₛ^{m}CₑₛA_{ds}^{m}C_{ds}G_{ds}^{m}C_{ds}^{m}C_{ds}^{m}C_{ds}^{m}C_{ds}T_{ds}G_{ds} | 87 |
| | T_{ds}^{m}Cₑₛ^{m}CₑₛAₑₛGₑₛ^{m}Cₑ | |

### 17. Angiopoietin-Like 3

Diabetes and obesity (sometimes collectively referred to as “diabesity”) are interrelated in that obesity is known to exacerbate the pathology of diabetes and greater than 60% of diabetics are obese. Most human obesity is associated with insulin resistance and leptin resistance. In fact, it has been suggested that obesity may have an even greater impact on insulin action than diabetes itself (Sindelka et al., Physiol Res., 2002, 51, 85-91). Additionally, several compounds on the market for the treatment of diabetes are known to induce weight gain, a very undesirable side effect to the treatment of this disease.

Cardiovascular disease is also interrelated to obesity and diabetes. Cardiovascular disease encompasses a wide variety of etiologies and has an equally wide variety of causative agents and interrelated players. Many causative agents contribute to symptoms such as elevated plasma levels of cholesterol, including non-high density lipoprotein cholesterol (non-HDL-C), as well as other lipid-related disorders. Such lipid-related disorders, generally referred to as dyslipidemia, include hyperlipidemia, hypercholesterolemia and hypertriglyceridemia among other indications. Elevated non-HDL cholesterol is associated with atherogenesis and its sequelae, including cardiovascular diseases such as arteriosclerosis, coronary artery disease, myocardial infarction, ischemic stroke, and other forms of heart disease. These rank as the most prevalent types of illnesses in industrialized countries. Indeed, an estimated 12 million people in the United States suffer with coronary artery disease and about 36 million require treatment for elevated cholesterol levels.

Epidemiological and experimental evidence has shown that high levels of circulating triglyceride (TG) can contribute to cardiovascular disease and a myriad of metabolic disorders (Valdivielso et al., 2009, Atherosclerosis Zhang et al., 2008, Circ Res. 1; 102(2):250-6). TG derived from either exogenous or endogenous sources is incorporated and secreted in chylomicrons from the intestine or in very low density lipoproteins (VLDL) from the liver. Once in circulation, TG is hydrolyzed by lipoprotein lipase (LpL) and the resulting free fatty acids can then be taken up by local tissues and used as an energy source. Due to the profound effect LpL has on plasma TG and metabolism in general, discovering and developing compounds that affect LpL activity are of great interest.

Metabolic syndrome is a combination of medical disorders that increase one's risk for cardiovascular disease and diabetes. The symptoms, including high blood pressure, high triglycerides, decreased HDL and obesity, tend to appear together in some individuals. It affects a large number of people in a clustered fashion. In some studies, the prevalence in the USA is calculated as being up to 25% of the population. Metabolic syndrome is known under various other names, such as (metabolic) syndrome X, insulin resistance syndrome, Reaven's syndrome or CHAOS. With the high prevalence of cardiovascular disorders and metabolic disorders there remains a need for improved approaches to treat these conditions

The angiopoietins are a family of secreted growth factors. Together with their respective endothelium-specific receptors, the angiopoietins play important roles in angiogenesis. One family member, angiopoietin-like 3 (also known as angiopoietin-like protein 3, ANGPT5, ANGPTL3, or angiopoietin 5), is predominantly expressed in the liver, and is thought to play a role in regulating lipid metabolism (Kaplan et al., J. Lipid Res., 2003, 44, 136-143). Genome-wide association scans (GWAS) surveying the genome for common variants associated with plasma concentrations of HDL, LDL and triglyceride found an association between triglycerides and single-nucleotide polymorphisms (SNPs) near ANGPTL3 (Willer et al., Nature Genetics, 2008, 40(2):161-169). Individuals with homozygous ANGPTL3 loss-of-function mutations present with low levels of all atherogenic plasma lipids and lipoproteins, such as total cholesterol (TC) and TG, low density lipoprotein cholesterol (LDL-C), apoliprotein B (apoB), non-HDL-C, as well as HDL-C (Romeo et al. 2009, J Clin Invest, 119(1):70-79; Musunuru et al. 2010 N Engl J Med, 363:2220-2227; Martin-Campos et al. 2012, Clin Chim Acta, 413:552-555; Minicocci et al. 2012, J Clin Endocrinol Metab, 97:e1266-1275; Noto et al. 2012, Arterioscler Thromb Vasc Biol, 32:805-809; Pisciotta et al. 2012, Circulation Cardiovasc Genet, 5:42-50). This clinical phenotype has been termed familial combined hypolipidemia (FHBL2). Despite reduced secretion of VLDL, subjects with FHBL2 do not have increased hepatic fat content. They also appear to have lower plasma glucose and insulin levels, and importantly, both diabetes and cardiovascular disease appear to be absent from these subjects. No adverse clinical phenotypes have been reported to date (Minicocci et al. 2013, J of Lipid Research, 54:3481-3490). Reduction of ANGPTL3 has been shown to lead to a decrease in TG, cholesterol and LDL levels in animal models (U.S. Ser. No. 13/520,997; PCT Publication WO 2011/085271). Mice deficient in ANGPTL3 have very low plasma triglyceride (TG) and cholesterol levels, while overpexpression produces the opposite effects (Koishi et al. 2002; Koster 2005; Fujimoto 2006). Accordingly, the potential role of ANGPTL3 in lipid metabolism makes it an attractive target for therapeutic intervention.

### Oligonucleotides Designed to Target Human Angiopoietin-Like 3 (ANGPTL3)

TABLE 18

| | | SEQ |
|---|---|---|
| ISIS | | ID |
| No. | Sequence (5′ to 3′) | No. |
| 563580 | GₑₛGₑₛAₑₛ^{m}CₑₛAₑₛT_{ds}T_{ds}G_{ds}^{m}C_{ds}^{m}C_{ds}A_{ds}G_{ds}T_{ds}A_{ds}A_{ds} | 88 |
| | Tₑₛ^{m}CₑₛGₑₛ^{m}CₑₛAₑ | |

### 18. Plasma Prekallikrein (PKK)

Plasma prekallikrein (PKK) is the precursor of plasma kallikrein (PK), which is encoded by the KLKB1 gene. PKK is a glycoprotein that participates in the surface-dependent activation of blood coagulation, fibrinolysis, kinin generation, and inflammation. PKK is converted to PK by Factor XIIa by the cleavage of an internal Arg-Ile peptide bond. PK liberates kinins from kininogens and also generates plasmin from plasminogen. PK is a member of the kinin-kallikrein pathway, which consists of several proteins that play a role in inflammation, blood pressure control, coagulation, and pain.

Oligonucleotides designed to target human Plasma prekallikrein (PKK)Oligonucleotides designed to

TABLE 19

| | | SEQ |
|---|---|---|
| Isis | | ID |
| No. | Sequence (5' to 3') | No. |
| 546254 | TₑₛGₑₛ^{m}CₑₛAₑₛAₑₛG_{ds}T_{ds}^{m}C_{ds}T_{ds}^{m}C_{ds}T_{ds}T_{ds}G_{ds}G_{ds}^{m}C_{ds}Aₑₛ | 89 |
| | AₑₛAₑₛ^{m}CₑₛAₑ | |

### 19. GHR

Growth hormone is produced in the pituitary and secreted into the bloodstream where it binds to growth hormone receptor (GHR) on many cell types, causing production of insulin-like growth factor-1 (IGF-1). IGF-1 is produced mainly in the liver, but also in adipose tissue and the kidney, and secreted into the bloodstream. Several disorders, such as acromegaly and gigantism, are associated with elevated growth hormone levels and/or elevated IGF-I levels in plasma and/or tissues.

Excessive production of growth hormone can lead to diseases such as acromegaly or gigantism. Acromegaly and gigantism are associated with excess growth hormone, often caused by a pituitary tumor, and affects 40-50 per million people worldwide with about 15,000 patients in each of the US and Europe and an annual incidence of about 4-5 per million people. Acromegaly and gigantism are initially characterized by abnormal growth of the hands and feet and bony changes in the facial features. Many of the growth related outcomes are mediated by elevated levels of serum IGF-1.

Embodiments provided herein relate to methods, compounds, and compositions for treating, preventing, or ameliorating a disease associated with excess growth hormone. Several embodiments provided herein are drawn to antisense compounds or oligonucleotides targeted to growth hormone receptor (GHR). Several embodiments are directed to treatment, prevention, or amelioration of acromegaly with antisense compounds or oligonucleotides targeted to growth hormone receptor (GHR).

TABLE 20
Oligonucleotides designed to target growth
hormone receptor (GHR)

| | | SEQ |
|---|---|---|
| Isis | | ID |
| No. | Sequence (5′ to 3′) | No. |
| 532254 | AₑₛGₑₛ^{m}CₑₛAₑₛTₑₛA_{ds}G_{ds}A_{ds}T_{ds}T_{ds}T_{ds}T_{ds}G_{ds}A_{ds}^{m}C_{ds}Tₑₛ | 90 |
| | Tₑₛ^{m}Cₑₛ^{m}Cₑₛ^{m}Cₑ | |
| 532401 | ^{m}Cₑₛ^{m}CₑₛAₑₛ^{m}Cₑₛ^{m}CₑₛT_{ds}T_{ds}T_{ds}G_{ds}G_{ds}G_{ds}T_{ds}G_{ds}A_{ds}A_{ds} | 91 |
| | TₑₛAₑₛGₑₛ^{m}CₑₛAₑ | |
| 523723 | Aₑₛ^{m}CₑₛTₑₛ^{m}CₑₛAₑₛA_{ds}^{m}C_{ds}T_{ds}T_{ds}G_{ds}A_{ds}G_{ds}G_{ds}A_{ds}^{m}C_{ds} | 92 |
| | AₑₛAₑₛTₑₛAₑₛAₑ | |
| 541767 | AₑₛGₑₛ^{m}CₖₛT_{ds}G_{ds}A_{ds}A_{ds}G_{ds}G_{ds}^{m}C_{ds}A_{ds}A_{ds}^{m}C_{ds}^{m}CₖₛAₖₛGₑ | 93 |
| 541875 | AₑₛGₑₛAₖₛG_{ds}T_{ds}T_{ds}G_{ds}A_{ds}^{m}C_{ds}A_{ds}T_{ds}G_{ds}G_{ds}Gₖₛ^{m}CₖₛAₑ | 94 |
| 542112 | ^{m}Cₑₛ^{m}CₑₛAₖₛG_{ds}T_{ds}A_{ds}G_{ds}T_{ds}^{m}C_{ds}A_{ds}A_{ds}T_{ds}A_{ds}TₖₛTₖₛAₑ | 95 |
| 542118 | ^{m}CₑₛTₑₛ^{m}CₖₛA_{ds}A_{ds}^{m}C_{ds}T_{ds}T_{ds}G_{ds}A_{ds}G_{ds}G_{ds}A_{ds}^{m}CₖₛAₖₛAₑ | 96 |
| 542185 | AₑₛGₑₛTₖₛA_{ds}T_{ds}A_{ds}T_{ds}T_{ds}^{m}C_{ds}A_{ds}G_{ds}T_{ds}^{m}C_{ds}^{m}CₖₛAₖₛAₑ | 97 |

### F. Certain Nucleic Acid GalNAc Conjugates

In certain embodiments, conjugated antisense compounds comprise antisense compounds having the nucleobase sequence of the antisense compounds in Table 21 below attached to a GalNAc conjugate. In certain embodiments, conjugated antisense compounds comprise antisense compounds having the nucleobase sequence and chemical modifications of the antisense compounds in Table 21 below attached to a GalNAc conjugate. All internucleoside linkages are phosphorothioate internucleoside linkages unless otherwise indicated. A subscript “1” indicates an LNA bicyclic nucleoside. A subscript “d” indicates a 2′-deoxy nucleoside. A subscript “e” indicates a 2′-MOE modified nucleoside. A subscript “v” indicates a 2-amino-2-deoxyadenosine.

TABLE 21

| | | | | | SEQ |
|---|---|---|---|---|---|
| Sequence | | | | Intemucleoside | ID |
| 5′ to 3′ | Target | Motif | Chemistry | Linkages | NO |
| TₗGₗGₗC_{d}A_{d}A_{d}G_{d}C_{d}A_{d}T_{d}C_{d}C_{d}TₗGₗTₗA_{d} | HIF-1α | 3-9-3-1 | LNA/deoxy | phosphorothioate | 98 |
| CₗTₗCₗAₗA_{d}T_{d}C_{d}C_{d}A_{d}T_{d}G_{d}G_{d}CₗAₗGₗC_{d} | Survivin | 4-8-3-1 | LNA/deoxy | phosphorothioate | 99 |
| AₗCₗCₗA_{d}A_{d}G_{d}T_{d}T_{d}T_{d}C_{d}T_{d}T_{d}C_{d}AₗGₗCₗ | Androgen | 3-10-3 | LNA/deoxy | phosphorothioate | 100 |
| | Receptor | | | | |
| GₗCₗA_{d}T_{d}T_{d}G_{d}G_{d}T_{d}A_{d}T_{d}TₗCₗAₗ | ApoB | 2-8-3 | LNA/deoxy | phosphorothioate | 101 |
| TₗTₗCₗAₗGₗC_{d}A_{d}T_{d}T_{d}G_{d}G_{d}T_{d}A_{d}T_{d}T_{d}CₗAₗ | ApoB | 5-10-5 | LNA/deoxy | phosphorothioate | 102 |
| GₗTₗGₗ | | | | | |
| CₗAₗGₗICA_{d}T_{d}T_{d}G_{d}G_{d}T_{d}A_{d}T_{d}TₗCₗAₗG_{d} | ApoB | 3-10-3 | LNA/deoxy | phosphorothioate | 103 |
| CₗAₗGₗCA_{d}T_{d}T_{d}G_{d}G_{d}T_{d}A_{d}T_{d}TₗCₗAₗ | ApoB | 3-9-3 | LNA/deoxy | phosphorothioate | 104 |
| AₗGₗCₗA_{d}T_{d}T_{d}G_{d}G_{d}T_{d}A_{d}T_{d}TₗCₗAₗ | ApoB | 3-8-3 | LNA/deoxy | phosphorothioate | 105 |
| GₗCₗA_{d}T_{d}T_{d}G_{d}G_{d}T_{d}A_{d}T_{d}TₗCₗ | ApoB | 2-8-2 | LNA/deoxy | phosphorothioate | 106 |
| TₗGₗCₗT_{d}A_{d}C_{d}A_{d}A_{d}A_{d}A_{d}CaCₗCₗAₗ | PCSK9 | 3-8-3 | LNA/deoxy | phosphorothioate | 107 |
| CₗcC_{d}AₗT_{d}T_{d}GₗTₗC_{d}A_{d}CₗA_{d}CₗT_{d}CₗCₗ | miR-122 | | LNA/deoxy | phosphorothioate | 108 |
| CGGCATGTCTATTTTGTA | TGF-β2 | | | phosphorothioate | 109 |
| GGCTAAATCGCTCCACCAAG | RRM2 | | | phosphorothioate | 110 |
| CTCTAGCGTCTTAAAGCCGA | RRM1 | | | phosphorothioate | 111 |
| GCTGCATGATCTCCTTGGCG | AKT-1 | | | phosphorothioate | 112 |
| ACGTTGAGGGGCATCGTCGC | c-Myc | | | Morpholino | 113 |
| CGGTTAGAAGACTCATCTTT | Influenza | | | Morpholino | 114 |
| | PB1-AUG | | | | |
| CTCCAACATCAAGGAAGATGGCA | dystrophin | | | Morpholino | 115 |
| TTTCTAG | | | | | |
| GAATATTAACANACTGACAAGTC | Marburg | | | Morpholino | 116 |
| | virus NP | | | | |
| CGTTGATANTTCTGCCATNCT | Marburg | | | Morpholino | 117 |
| | virus VP24 | | | | |
| GCCATGGTTTTTTCTCAGG | Ebola virus | | | Morpholino | 118 |
| | VP24 | | | | |
| CCTGCCCTTTGTTCTAGTTG | Ebola virus | | | Morpholino | 119 |
| | VP35 | | | | |
| GGGTCTGCAᵥGCGGGAᵥTGGT | CCR3 & | | | phosphorothioate | 120 |
| | CSF2RB | | | | |
| GTTAᵥCTAᵥCTTCCAᵥCCTGCCTG | CCR3 & | | | phosphorothioate | 121 |
| | CSF2RB | | | | |
| TATCCGGAGGGCTCGCCATGCTGCT | IRS-1 | | | phosphorothioate | 122 |
| GTCGCCCCTTCTCCCCGCAGC | Smad7 | | | phosphorothioate | 123 |
| GGACCCTCCTCCGGAGCC | IGF-1R | | | phosphorothioate | 124 |
| ACCAGGCGTCTCGTGGGGCACAT | Ki-67 | | | phosphorothioate | 125 |
| TCTCCCAGCGTGCGCCAT | BCL-2 | | | phosphorothioate | 126 |
| GTGCTCCATTGATGC | c-Raf | | | phosphate | 127 |
| TₑCₑCₑCₑGₑCₑCTGTGACATₑGₑCₑAₑTₑTₑ | c-Raf | 6-8-6 | MOE/deoxy | | 128 |
| CₑAₑGₑCₑAGCAGAGTCTTCATₑCₑAₑTₑ | Clusterin | 4-13-4 | MOE/deoxy | | 129 |
| GₑGₑGₑAₑC_{d}G_{d}C_{d}G_{d}G_{d}C_{d}G_{d}C_{d}T_{d}C_{d}G_{d}G_{d} | HSPB1 | 4-12-4 | MOE/deoxy | | 130 |
| TₑCₑAₑTₑ | | | | | |
| CₑCₑAₑCₑAₑA_{d}G_{d}C_{d}T_{d}G_{d}T_{d}C_{d}C_{d}A_{d}G_{d}Tₑ | CTGF | 5-10-5 | MOE/deoxy | | 131 |
| CₑTₑAₑAₑ | | | | | |
| CₑCₑGₑC_{d}A_{d}G_{d}C_{d}C_{d}A_{d}T_{d}G_{d}C_{d}GₑCₑTₑCₑ | CD49d/ | 3-9-8 | MOE/deoxy | | 132 |
| TₑTₑGₑGₑ | VLA-4 | | | | |
| TₑCₑAₑGₑGₑG_{d}C_{d}A_{d}T_{d}T_{d}C_{d}T_{d}T_{d}T_{d}C_{d}Cₑ | GHR | 5-10-5 | MOE/deoxy | | 133 |
| AₑTₑTₑCₑ | | | | | |
| CₑGₑAₑAₑGₑG_{d}A_{d}A_{d}A_{d}C_{d}A_{d}A_{d}T_{d}A_{d}C_{d}Tₑ | IGF-1R | 5-10-5 | MOE/deoxy | | 134 |
| CₑCₑGₑAₑ | | | | | |
| GₑAₑCₑAₑGₑC_{d}A_{d}G_{d}C_{d}G_{d}C_{d}A_{d}G_{d}C_{d}AₑGₑ | hepcidin | 5-10-5 | MOE/deoxy | | 135 |
| AₑAₑAₑ | | | | | |
| TₑGₑGₑAₑAₑA_{d}G_{d}G_{d}C_{d}T_{d}T_{d}A_{d}T_{d}A_{d}C_{d}Cₑ | IL-4Rα1 | 5-10-5 | MOE/deoxy | | 136 |
| CₑCₑTₑCₑ | | | | | |
| TCAAGGAAGATGGCATTTCT | dystrophin | | 2′-O-Methyl | phosphorothioate | 137 |
| GUGGCUAACAGAAGCU | dystrophin | | 2′-O-Methyl | phosphorothioate | 138 |
| UUUGCCGCUGCCCAAUGCCAUCCUG | dystrophin | | 2′-O-Methyl | phosphorothioate | 139 |
| GₘCₘGₘUₘG_{d}C_{d}C_{d}T_{d}C_{d}C_{d}T_{d}C_{d}A_{d}C_{d}Uₘ | Protein | 4-10-4 | 2′-O- | phosphorothioate | 140 |
| GₘGₘCₘ | kinase A | | Methyl/deoxy | | |

Additional Sequences and Oligonucleotides Suitable for Conjugation with any Conjugate Herein

In certain embodiments, a compound comprises an antisense oligonucleotide targeted to eukaryotic Initiation Factor 4E (eIF4E) known in the art and a conjugate group described herein. In certain embodiments, antisense oligonucleotides targeted to dIF4E are RNAi (siRNA or ssRNA) compounds. In certain embodiments, antisense oligonucleotides targeted to dIF4E are RNase H based antisense compounds. Examples of antisense oligonucleotides targeted to eIF4E suitable for conjugation include but are not limited to those disclosed in U.S. Pat. No. 7,425,544, which is incorporated by reference in its entirety herein. In certain embodiments, a compound comprises an antisense oligonucleotide having a nucleobase sequence of any of SEQ ID NOs: 18-122 disclosed in U.S. Pat. No. 7,425,544 and a conjugate group described herein. In certain embodiments, a compound comprises an antisense strand having a nucleobase sequence of any of SEQ ID NOs: 212-459 disclosed in U.S. Pat. No. 7,425,544 and a conjugate group described herein. The nucleobase sequences of all of the aforementioned referenced SEQ ID NOs are incorporated by reference herein.

In certain embodiments, a compound comprises an antisense oligonucleotide targeted to Signal Transducer and Activator of Transcription 3 (STAT3) known in the art and a conjugate group described herein. In certain embodiments, antisense oligonucleotides targeted to STAT3 are RNAi (siRNA or ssRNA) compounds. In certain embodiments, antisense oligonucleotides targeted to STAT3 are RNase H based antisense compounds. Examples of antisense oligonucleotides targeted to STAT3 suitable for conjugation include but are not limited to those disclosed in WO 2012/135736, WO 2005/083124, and U.S. Pat. No. 6,727,064; which are incorporated by reference in their entireties herein. In certain embodiments, a compound comprises an antisense oligonucleotide having a nucleobase sequence of any of SEQ ID NOs: 9-426, 430-442, 445-464, 471-498, 500-1034, 1036-1512, and 1541-2757 disclosed in WO 2012/135736 and a conjugate group described herein.

In certain embodiments, a compound comprises an antisense oligonucleotide having a nucleobase sequence of any of SEQ ID NOs: 2-81, 108-150, and 159-381 disclosed in WO 2005/083124 and a conjugate group described herein. In certain embodiments, a compound comprises an antisense oligonucleotide having a nucleobase sequence of any of SEQ ID NOs: 2-81 and 108-150 disclosed in U.S. Pat. No. 6,727,064 and a conjugate group described herein. The nucleobase sequences of all of the aforementioned referenced SEQ ID NOs are incorporated by reference herein.

In certain embodiments, a compound comprises an antisense oligonucleotide targeted to glucocorticoid receptor (GCCR) known in the art and a conjugate group described herein. In certain embodiments, antisense oligonucleotides targeted to GCCR are RNAi (siRNA or ssRNA) compounds. In certain embodiments, antisense oligonucleotides targeted to GCCR are RNase H based antisense compounds. Examples of antisense oligonucleotides targeted to GCCR suitable for conjugation include but are not limited to those disclosed in WO 2005/071080, WO 2007/035759, and WO 2007/136988; which are incorporated by reference in their entireties herein. In certain embodiments, a compound comprises an antisense oligonucleotide having a nucleobase sequence of any of SEQ ID NOs: 30-216, and 306-310 disclosed in WO 2005/071080 and a conjugate group described herein. In certain embodiments, a compound comprises an antisense oligonucleotide having a nucleobase sequence of any of SEQ ID NOs: 26-113 disclosed in WO 2007/035759 and a conjugate group disclosed herein. In certain embodiments, a compound comprises an antisense oligonucleotide having a nucleobase sequence of any of SEQ ID NOs: 413-485 disclosed in WO 2007/136988 and a conjugate group disclosed herein. The nucleobase sequences of all of the aforementioned referenced SEQ ID NOs are incorporated by reference herein.

In certain embodiments, a compound comprises an antisense oligonucleotide targeted to glucagon receptor (GCGR) known in the art and a conjugate group described herein. In certain embodiments, antisense oligonucleotides targeted to GCGR are RNAi (siRNA or ssRNA) compounds. In certain embodiments, antisense oligonucleotides targeted to GCGR are RNase H based antisense compounds. Examples of antisense oligonucleotides targeted to GCGR suitable for conjugation include but are not limited to those disclosed in U.S. Pat. Nos. 7,750,142; 7,399,853; WO 2007/035771; and WO 2007/134014; which are incorporated by reference in their entireties herein. In certain embodiments, a compound comprises an antisense oligonucleotide having a nucleobase sequence of any of SEQ ID NOs: 20-399 disclosed in U.S. Pat. No. 7,750,142 and a conjugate group described herein. In certain embodiments, a compound comprises an antisense oligonucleotide having a nucleobase sequence of any of SEQ ID NOs: 20-399 disclosed in U.S. Pat. No. 7,399,853 and a conjugate group described herein. In certain embodiments, a compound comprises an antisense oligonucleotide having a nucleobase sequence of SEQ ID NO: 2 disclosed in WO 2007/035771 and a conjugate group described herein. In certain embodiments, a compound comprises an antisense oligonucleotide having a nucleobase sequence of any of SEQ ID NOs: 486-680 disclosed in WO 2007/134014 and a conjugate group described herein. The nucleobase sequences of all of the aforementioned referenced SEQ ID NOs are incorporated by reference herein.

In certain embodiments, a compound comprises an antisense oligonucleotide targeted to Protein Tyrosine Phosphatase 1B (PTP1B) known in the art and a conjugate group described herein. In certain embodiments, antisense oligonucleotides targeted to PTP1B are RNAi (siRNA or ssRNA) compounds. In certain embodiments, antisense oligonucleotides targeted to PT1B are RNase H based antisense compounds. Examples of antisense oligonucleotides targeted to PTP1B suitable for conjugation include but are not limited to those disclosed in U.S. Pat. No. 7,563,884 and WO 2007/131237, which are incorporated by reference in their entireties herein. In certain embodiments, a compound comprises an antisense oligonucleotide having a nucleobase sequence of any of SEQ ID NOs 17-96 and 244-389 disclosed in U.S. Pat. No. 7,563,884 and a conjugate group described herein. In certain embodiments, a compound comprises an antisense oligonucleotide having a nucleobase sequence of any of SEQ ID NOs 886-1552 disclosed in WO 2007/131237 and a conjugate group described herein. The nucleobase sequences of all of the aforementioned referenced SEQ ID NOs are incorporated by reference herein.

In certain embodiments, a compound comprises an antisense oligonucleotide targeted to Fibroblast Growth Factor Receptor 4 (FGFR4) known in the art and a conjugate group described herein. In certain embodiments, antisense oligonucleotides targeted to FGFR4 are RNAi (siRNA or ssRNA) compounds. In certain embodiments, antisense oligonucleotides targeted to FGFR4 are RNase H based antisense compounds. Examples of antisense oligonucleotides targeted to FGFR4 suitable for conjugation include but are not limited to those disclosed in WO 2009/046141, which is incorporated by reference in its entirety herein. In certain embodiments, a compound comprises an antisense oligonucleotide having a nucleobase sequence of any of SEQ ID NOs 21-24, 28, 29, 36, 38, 39, 43, 48, 51, 54-56, 58-60, 64-66, and 92-166 disclosed in WO 2009/046141 and a conjugate group described herein. The nucleobase sequences of all of the aforementioned referenced SEQ ID NOs are incorporated by reference herein.

In certain embodiments, a compound comprises an antisense oligonucleotide targeted to alpha-1-antitrypsin (A1AT) known in the art and a conjugate group described herein. In certain embodiments, antisense oligonucleotides targeted to A1AT are RNAi (siRNA or ssRNA) compounds. In certain embodiments, antisense oligonucleotides targeted to A1AT are RNase H based antisense compounds. Examples of antisense oligonucleotides targeted to A1AT suitable for conjugation include but are not limited to those disclosed in WO 2013/142514, which is incorporated by reference in its entirety herein. In certain embodiments, a compound comprises an antisense oligonucleotide having a nucleobase sequence of any of SEQ ID NOs 20-41 disclosed in WO 2013/142514 and a conjugate group described herein. The nucleobase sequences of all of the aforementioned referenced SEQ ID NOs are incorporated by reference herein.

In certain embodiments, a compound comprises an antisense oligonucleotide targeted to Factor VII known in the art and a conjugate group described herein. In certain embodiments, antisense oligonucleotides targeted to Factor VII are RNAi (siRNA or ssRNA) compounds. In certain embodiments, antisense oligonucleotides targeted to Factor VII are RNase H based antisense compounds. Examples of antisense oligonucleotides targeted to Factor VII suitable for conjugation include but are not limited to those disclosed in WO 2013/119979 and WO 2009/061851, which are incorporated by reference in their entireties herein. In certain embodiments, a compound comprises an antisense oligonucleotide having a nucleobase sequence of any of SEQ ID NOs 21-659 disclosed in WO 2013/119979 and a conjugate group described herein. In certain embodiments, a compound comprises an antisense oligonucleotide having a nucleobase sequence of any of SEQ ID NOs 4-159 and 168-611 disclosed in WO 2009/061851 and a conjugate group described herein. The nucleobase sequences of all of the aforementioned referenced SEQ ID NOs are incorporated by reference herein.

In certain embodiments, a compound comprises an antisense oligonucleotide targeted to Factor XI known in the art and a conjugate group described herein. In certain embodiments, antisense oligonucleotides targeted to Factor XI are RNAi (siRNA or ssRNA) compounds. In certain embodiments, antisense oligonucleotides targeted to Factor XI are RNase H based antisense compounds. Examples of antisense oligonucleotides targeted to Factor XI suitable for conjugation include but are not limited to those disclosed in WO 2010/045509 and WO 2010/121074, which are incorporated by reference in their entireties herein. In certain embodiments, a compound comprises an antisense oligonucleotide having a nucleobase sequence of any of SEQ ID NOs 15-270 disclosed in WO 2010/045509 and a conjugate group described herein. In certain embodiments, a compound comprises an antisense oligonucleotide having a nucleobase sequence of any of SEQ ID NOs 15-270 disclosed in WO 2010/121074 and a conjugate group described herein. The nucleobase sequences of all of the aforementioned referenced SEQ ID NOs are incorporated by reference herein.

In certain embodiments, a compound comprises an antisense oligonucleotide targeted to Hepatitis B Virus (HBV) known in the art and a conjugate group described herein. In certain embodiments, antisense oligonucleotides targeted to HBV are RNAi (siRNA or ssRNA) compounds. In certain embodiments, antisense oligonucleotides targeted to HBV are RNase H based antisense compounds. Examples of antisense oligonucleotides targeted to HBV suitable for conjugation include but are not limited to those disclosed in WO 2012/145697 and WO 2012/145697, which are incorporated by reference in their entireties herein. In certain embodiments, a compound comprises an antisense oligonucleotide having a nucleobase sequence of any of SEQ ID NOs 5-310, 321-802, 804-1272, 1288-1350, 1364-1372, 1375, 1376, and 1379 disclosed in WO 2012/145697 and a conjugate group described herein. In certain embodiments, a compound comprises an antisense oligonucleotide having a nucleobase sequence of any of SEQ ID NOs 14-22 disclosed in WO 2011/047312 and a conjugate group described herein. The nucleobase sequences of all of the aforementioned referenced SEQ ID NOs are incorporated by reference herein.

In certain embodiments, a compound comprises an antisense oligonucleotide targeted to transthyretin (TTR) known in the art and a conjugate group described herein. In certain embodiments, antisense oligonucleotides targeted to TTR are RNAi (siRNA or ssRNA) compounds.

In certain embodiments, antisense oligonucleotides targeted to TTR are RNase H based antisense compounds. Examples of antisense oligonucleotides targeted to TTR suitable for conjugation include but are not limited to those disclosed in WO 2011/139917 and U.S. Pat. No. 8,101,743, which are incorporated by reference in their entireties herein. In certain embodiments, a compound comprises an antisense oligonucleotide having a nucleobase sequence of any of SEQ ID NOs 8-160, 170-177 disclosed in WO 2011/139917 and a conjugate group described herein. In certain embodiments, a compound comprises an antisense oligonucleotide having a nucleobase sequence of any of SEQ ID NOs 12-89 disclosed in U.S. Pat. No. 8,101,743 and a conjugate group described herein. In certain embodiments, a compound comprises an antisense oligonucleotide having a nucleobase sequence complementary to a preferred target segment of any of SEQ ID NOs 90-133 disclosed in U.S. Pat. No. 8,101,743 and a conjugate group described herein. The nucleobase sequences of all of the aforementioned referenced SEQ ID NOs are incorporated by reference herein.

In certain embodiments, a compound comprises an antisense oligonucleotide targeted to apolipoprotein(a) (apo(a)) known in the art and a conjugate group described herein. In certain embodiments, antisense oligonucleotides targeted to apo(a) are RNAi (siRNA or ssRNA) compounds. In certain embodiments, antisense oligonucleotides targeted to apo(a) are RNase H based antisense compounds.

Examples of antisense oligonucleotides targeted to apo(a) suitable for conjugation include but are not limited to those disclosed in WO 2013/177468; U.S. Pat. Nos. 8,673,632; 7,259,150; and US Patent Application Publication No. US 2004/0242516; which are incorporated by reference in their entireties herein. In certain embodiments, a compound comprises an antisense oligonucleotide having a nucleobase sequence of any of SEQ ID NOs 12-130, 133, 134 disclosed in WO 2013/177468 and a conjugate group described herein. In certain embodiments, a compound comprises an antisense oligonucleotide having a nucleobase sequence of any of SEQ ID NOs 11-45 and 85-96 disclosed in U.S. Pat. No. 8,673,632 and a conjugate group described herein. In certain embodiments, a compound comprises an antisense oligonucleotide having a nucleobase sequence of any of SEQ ID NOs 11-45 disclosed in U.S. Pat. No. 7,259,150 and a conjugate group described herein. In certain embodiments, a compound comprises an antisense oligonucleotide having a nucleobase sequence of any of SEQ ID NOs 7-41 disclosed in US Patent Application Publication No. US 2004/0242516 and a conjugate group described herein. The nucleobase sequences of all of the aforementioned referenced SEQ ID NOs are incorporated by reference herein.

In certain embodiments, a compound comprises an antisense oligonucleotide targeted to Apolipoprotein B (ApoB) known in the art and a conjugate group described herein. In certain embodiments, antisense oligonucleotides targeted to ApoB are RNAi (siRNA or ssRNA) compounds. In certain embodiments, antisense oligonucleotides targeted to ApoB are RNase H based antisense compounds. Examples of antisense oligonucleotides targeted to ApoB suitable for conjugation include but are not limited to those disclosed in US Patent Application Publication Nos. US 2010/0331390, US 2009/0306180, and US 2005/0009088; which are incorporated by reference in their entireties herein. In certain embodiments, a compound comprises an antisense oligonucleotide having a nucleobase sequence of SEQ ID NO: 20 disclosed in US 2010/0331390 and a conjugate group described herein. In certain embodiments, a compound comprises an antisense oligonucleotide having a nucleobase sequence of any of SEQ ID NOs 16-213 disclosed in US 2009/0306180 and a conjugate group described herein. In certain embodiments, a compound comprises an antisense oligonucleotide having a nucleobase sequence of any of SEQ ID NOs 17-70, 124-317, 319-333, 335-502, 504-804, and 864-887 disclosed in US 2005/0009088 and a conjugate group described herein. The nucleobase sequences of all of the aforementioned referenced SEQ ID NOs are incorporated by reference herein.

In certain embodiments, a compound comprises an antisense oligonucleotide targeted to Apolipoprotein C-III (ApoC-III) known in the art and a conjugate group described herein. In certain embodiments, antisense oligonucleotides targeted to ApoC-III are RNAi (siRNA or ssRNA) compounds. In certain embodiments, antisense oligonucleotides targeted to ApoC-III are RNase H based antisense compounds. Examples of antisense oligonucleotides targeted to ApoC-III suitable for conjugation include but are not limited to those disclosed in US Patent Application Publication No. US 2013/0317085, which is incorporated by reference in its entirety herein. In certain embodiments, a compound comprises an antisense oligonucleotide having a nucleobase sequence of any of SEQ ID NOs 19-96 and 209-221 disclosed in US 2013/0317085 and a conjugate group described herein. The nucleobase sequences of all of the aforementioned referenced SEQ ID NOs are incorporated by reference herein.

In certain embodiments, a compound comprises an antisense oligonucleotide targeted to proprotein convertase subtilisin/kexin type 9 (PCSK9) known in the art and a conjugate group described herein. In certain embodiments, antisense oligonucleotides targeted to PCSK9 are RNAi (siRNA or ssRNA) compounds. In certain embodiments, antisense oligonucleotides targeted to PCSK9 are RNase H based antisense compounds. Examples of antisense oligonucleotides targeted to PCSK9 suitable for conjugation include but are not limited to those disclosed in U.S. Pat. Nos. 8,143,230 and 8,664,190; which are incorporated by reference in their entireties herein. In certain embodiments, a compound comprises an antisense oligonucleotide having a nucleobase sequence of any of SEQ ID NOs 329-403 disclosed in U.S. Pat. No. 8,143,230 and a conjugate group described herein. In certain embodiments, a compound comprises an antisense oligonucleotide having a nucleobase sequence of any of SEQ ID NOs 4-455 and 458-461 disclosed in U.S. Pat. No. 8,664,190 and a conjugate group described herein. The nucleobase sequences of all of the aforementioned referenced SEQ ID NOs are incorporated by reference herein.

In certain embodiments, a compound comprises an antisense oligonucleotide targeted to C-reactive protein (CRP) known in the art and a conjugate group described herein. In certain embodiments, antisense oligonucleotides targeted to CRP are RNAi (siRNA or ssRNA) compounds. In certain embodiments, antisense oligonucleotides targeted to CRP are RNase H based antisense compounds. Examples of antisense oligonucleotides targeted to CRP suitable for conjugation include but are not limited to those disclosed in WO 2003/010284, WO 2005/005599, and WO 2007/143317; which are incorporated by reference in their entireties herein. In certain embodiments, a compound comprises an antisense oligonucleotide having a nucleobase sequence of any of SEQ ID NOs 10-63 disclosed in WO 2003/010284 and a conjugate group described herein.

In certain embodiments, a compound comprises an antisense oligonucleotide having a nucleobase sequence of any of SEQ ID NOs 19-72, 76-259, and 598-613 disclosed in WO 2005/005599 and a conjugate group described herein. In certain embodiments, a compound comprises an antisense oligonucleotide having a nucleobase sequence of any of SEQ ID NOs 409-412 disclosed in WO 2007/143317 and a conjugate group described herein. The nucleobase sequences of all of the aforementioned referenced SEQ ID NOs are incorporated by reference herein.

### G. Certain Pharmaceutical Compositions

In certain embodiments, the present disclosure provides pharmaceutical compositions comprising one or more antisense compound. In certain embodiments, such pharmaceutical composition comprises a suitable pharmaceutically acceptable diluent or carrier. In certain embodiments, a pharmaceutical composition comprises a sterile saline solution and one or more antisense compound. In certain embodiments, such pharmaceutical composition consists of a sterile saline solution and one or more antisense compound. In certain embodiments, the sterile saline is pharmaceutical grade saline. In certain embodiments, a pharmaceutical composition comprises one or more antisense compound and sterile water. In certain embodiments, a pharmaceutical composition consists of one or more antisense compound and sterile water. In certain embodiments, the sterile saline is pharmaceutical grade water. In certain embodiments, a pharmaceutical composition comprises one or more antisense compound and phosphate-buffered saline (PBS). In certain embodiments, a pharmaceutical composition consists of one or more antisense compound and sterile phosphate-buffered saline (PBS). In certain embodiments, the sterile saline is pharmaceutical grade PBS.

In certain embodiments, antisense compounds may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

Pharmaceutical compositions comprising antisense compounds encompass any pharmaceutically acceptable salts, esters, or salts of such esters. In certain embodiments, pharmaceutical compositions comprising antisense compounds comprise one or more oligonucleotide which, upon administration to an animal, including a human, is capable of providing (directly or indirectly) the biologically active metabolite or residue thereof. Accordingly, for example, the disclosure is also drawn to pharmaceutically acceptable salts of antisense compounds, prodrugs, pharmaceutically acceptable salts of such prodrugs, and other bioequivalents. Suitable pharmaceutically acceptable salts include, but are not limited to, sodium and potassium salts.

A prodrug can include the incorporation of additional nucleosides at one or both ends of an oligonucleotide which are cleaved by endogenous nucleases within the body, to form the active antisense oligonucleotide.

Lipid moieties have been used in nucleic acid therapies in a variety of methods. In certain such methods, the nucleic acid is introduced into preformed liposomes or lipoplexes made of mixtures of cationic lipids and neutral lipids. In certain methods, DNA complexes with mono- or poly-cationic lipids are formed without the presence of a neutral lipid. In certain embodiments, a lipid moiety is selected to increase distribution of a pharmaceutical agent to a particular cell or tissue. In certain embodiments, a lipid moiety is selected to increase distribution of a pharmaceutical agent to fat tissue. In certain embodiments, a lipid moiety is selected to increase distribution of a pharmaceutical agent to muscle tissue.

In certain embodiments, pharmaceutical compositions provided herein comprise one or more modified oligonucleotides and one or more excipients. In certain such embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, a pharmaceutical composition provided herein comprises a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, a pharmaceutical composition provided herein comprises one or more tissue-specific delivery molecules designed to deliver the one or more pharmaceutical agents of the present disclosure to specific tissues or cell types. For example, in certain embodiments, pharmaceutical compositions include liposomes coated with a tissue-specific antibody.

In certain embodiments, a pharmaceutical composition provided herein comprises a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, a pharmaceutical composition provided herein is prepared for oral administration. In certain embodiments, pharmaceutical compositions are prepared for buccal administration.

In certain embodiments, a pharmaceutical composition is prepared for administration by injection (e.g., intravenous, subcutaneous, intramuscular, etc.). In certain of such embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes. Aqueous injection suspensions may contain substances that increase the viscosity of the suspension, such as sodium carboxymethyl cellulose, sorbitol, or dextran. Optionally, such suspensions may also contain suitable stabilizers or agents that increase the solubility of the pharmaceutical agents to allow for the preparation of highly concentrated solutions.

In certain embodiments, a pharmaceutical composition is prepared for transmucosal administration. In certain of such embodiments penetrants appropriate to the barrier to be permeated are used in the formulation. Such penetrants are generally known in the art.

In certain embodiments, a pharmaceutical composition provided herein comprises an oligonucleotide in a therapeutically effective amount. In certain embodiments, the therapeutically effective amount is sufficient to prevent, alleviate or ameliorate symptoms of a disease or to prolong the survival of the subject being treated. Determination of a therapeutically effective amount is well within the capability of those skilled in the art.

In certain embodiments, one or more modified oligonucleotide provided herein is formulated as a prodrug. In certain embodiments, upon in vivo administration, a prodrug is chemically converted to the biologically, pharmaceutically or therapeutically more active form of an oligonucleotide. In certain embodiments, prodrugs are useful because they are easier to administer than the corresponding active form. For example, in certain instances, a prodrug may be more bioavailable (e.g., through oral administration) than is the corresponding active form. In certain instances, a prodrug may have improved solubility compared to the corresponding active form. In certain embodiments, prodrugs are less water soluble than the corresponding active form. In certain instances, such prodrugs possess superior transmittal across cell membranes, where water solubility is detrimental to mobility. In certain embodiments, a prodrug is an ester. In certain such embodiments, the ester is metabolically hydrolyzed to carboxylic acid upon administration. In certain instances the carboxylic acid containing compound is the corresponding active form. In certain embodiments, a prodrug comprises a short peptide (polyaminoacid) bound to an acid group. In certain of such embodiments, the peptide is cleaved upon administration to form the corresponding active form.

In certain embodiments, the present disclosure provides compositions and methods for reducing the amount or activity of a target nucleic acid in a cell. In certain embodiments, the cell is in an animal. In certain embodiments, the animal is a mammal. In certain embodiments, the animal is a rodent. In certain embodiments, the animal is a primate. In certain embodiments, the animal is a non-human primate. In certain embodiments, the animal is a human.

In certain embodiments, the present disclosure provides methods of administering a pharmaceutical composition comprising an oligonucleotide of the present disclosure to an animal. Suitable administration routes include, but are not limited to, oral, rectal, transmucosal, intestinal, enteral, topical, suppository, through inhalation, intrathecal, intracerebroventricular, intraperitoneal, intranasal, intraocular, intratumoral, and parenteral (e.g., intravenous, intramuscular, intramedullary, and subcutaneous). In certain embodiments, pharmaceutical intrathecals are administered to achieve local rather than systemic exposures. For example, pharmaceutical compositions may be injected directly in the area of desired effect (e.g., into the liver).

### Nonlimiting Disclosure and Incorporation by Reference

While certain compounds, compositions and methods described herein have been described with specificity in accordance with certain embodiments, the following examples serve only to illustrate the compounds described herein and are not intended to limit the same. Each of the references, GenBank accession numbers, and the like recited in the present application is incorporated herein by reference in its entirety.

Certain compounds, compositions, and methods herein are described as “comprising exactly” or “comprises exactly” a particular number of a particular element or feature. Such descriptions are used to indicate that while the compound, composition, or method may comprise additional other elements, the number of the particular element or feature is the identified number. For example, “a conjugate comprising exactly one GalNAc” is a conjugate that contains one and only one GalNAc, though it may contain other elements in addition to the one GalNAc.

Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar (2′-OH for the natural 2′-H of DNA) or as an RNA having a modified base (thymine (methylated uracil) for natural uracil of RNA).

Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, an oligonucleotide having the nucleobase sequence “ATCGATCG” encompasses any oligonucleotides having such nucleobase sequence, whether modified or unmodified, including, but not limited to, such compounds comprising RNA bases, such as those having sequence “AUCGAUCG” and those having some DNA bases and some RNA bases such as “AUCGATCG” and oligonucleotides having other modified bases, such as “AT^{me}CGAUCG,” wherein ^{me}C indicates a cytosine base comprising a methyl group at the 5-position.

### EXAMPLES

The following examples illustrate certain embodiments of the present disclosure and are not limiting. Moreover, where specific embodiments are provided, the inventors have contemplated generic application of those specific embodiments. For example, disclosure of an oligonucleotide having a particular motif provides reasonable support for additional oligonucleotides having the same or similar motif. And, for example, where a particular high-affinity modification appears at a particular position, other high-affinity modifications at the same position are considered suitable, unless otherwise indicated.

### Example 1: General Method for the Preparation of an Oligomeric Compound Comprising a GalNAc₃-3 Conjugate at the 5′ Terminus

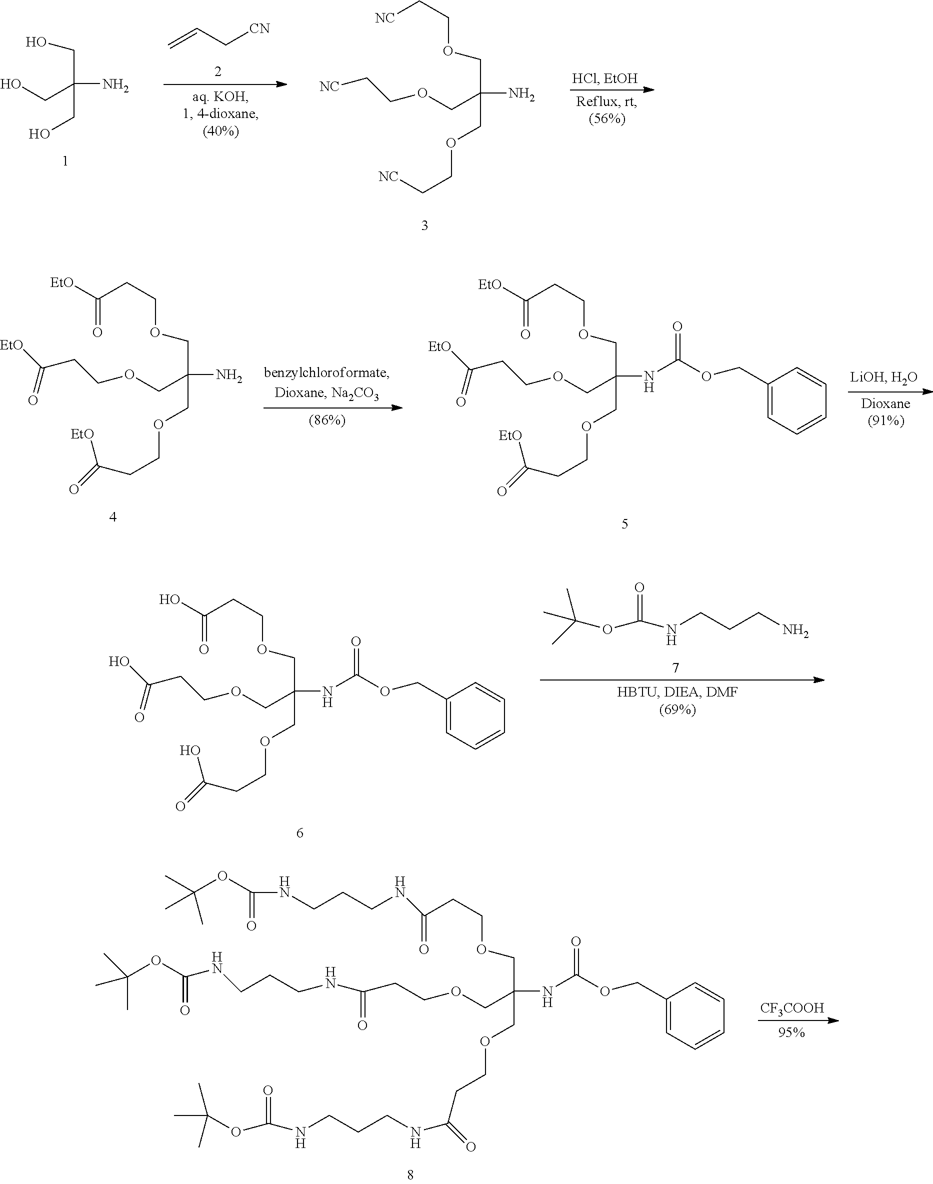

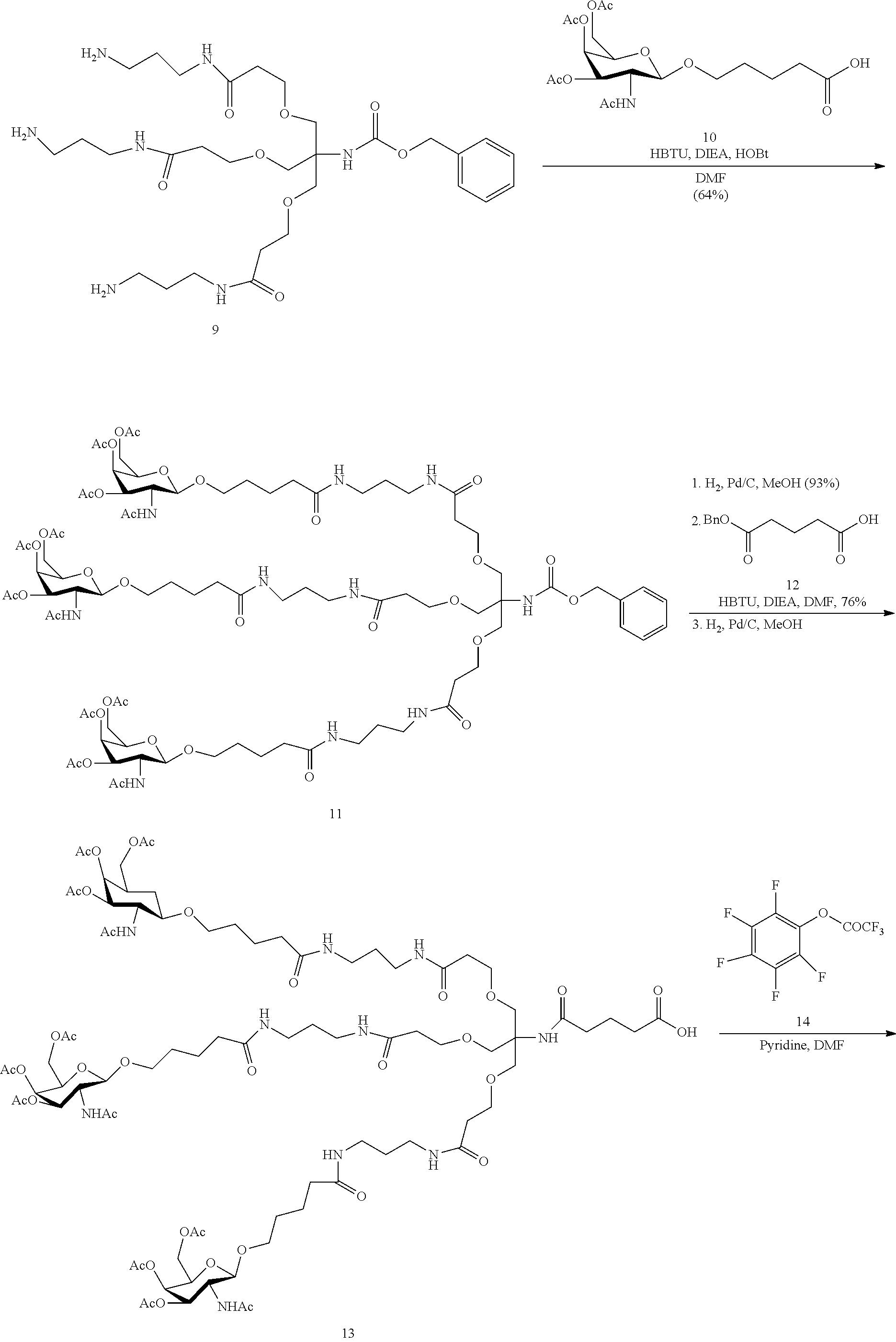

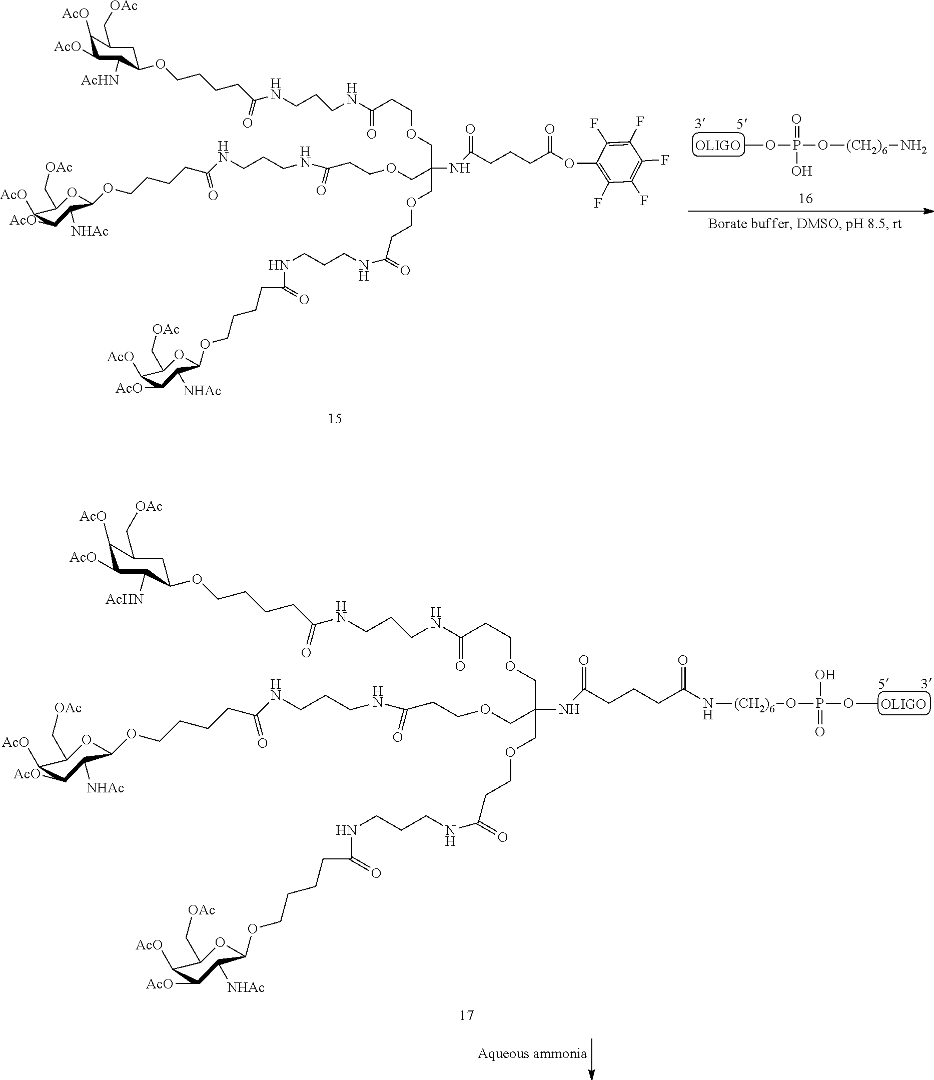

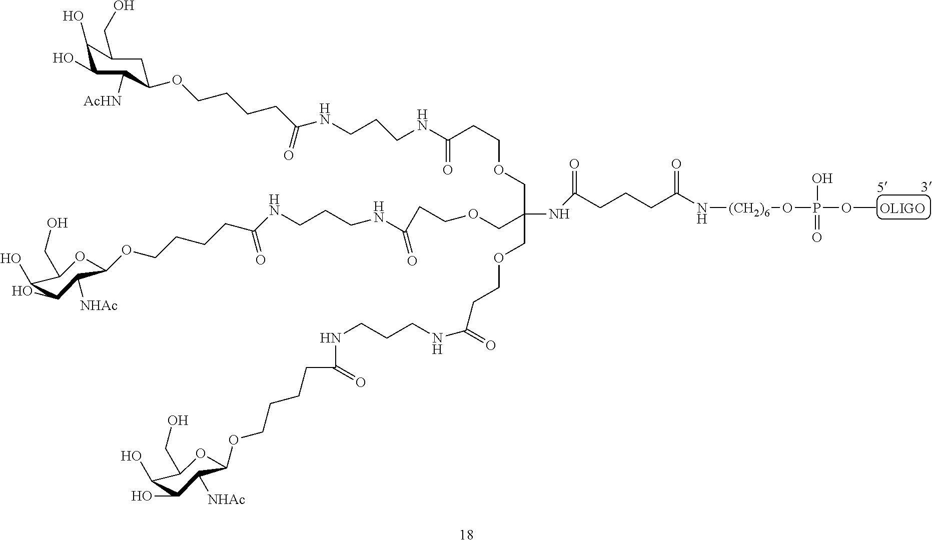

Compounds 1, 2, 7, 12, and 14 are commercially available. Compound 10 was prepared using similar procedures reported by Rensen et al., J. Med. Chem., 2004, 47, 5798-5808. Oligomeric compound 16 comprising a phosphodiester linked hexylamine is prepared using standard oligonucleotide synthesis procedures. Treatment of the protected oligomeric compound with aqueous ammonia provided the 5′-GalNAc₃-3 conjugated oligomeric compound (18). The GalNAc₃ cluster portion of the conjugate group GalNAc₃-3 (GalNAc₃-3ₐ) can be combined with any cleavable moiety to provide a variety of conjugate groups, wherein GalNAc₃-3ₐ has the formula:

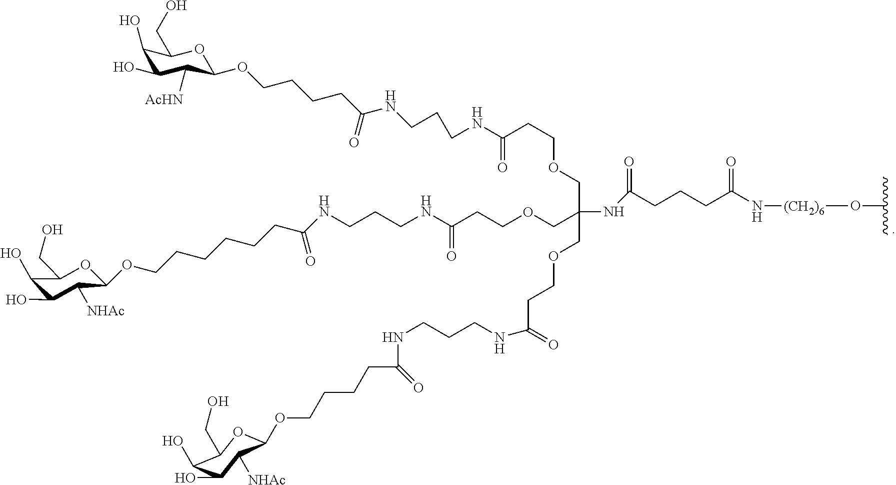

### Example 2: General Method for the Preparation of an Oligomeric Compound Comprising a GalNAc₃-7 Conjugate at the 5′ Terminus

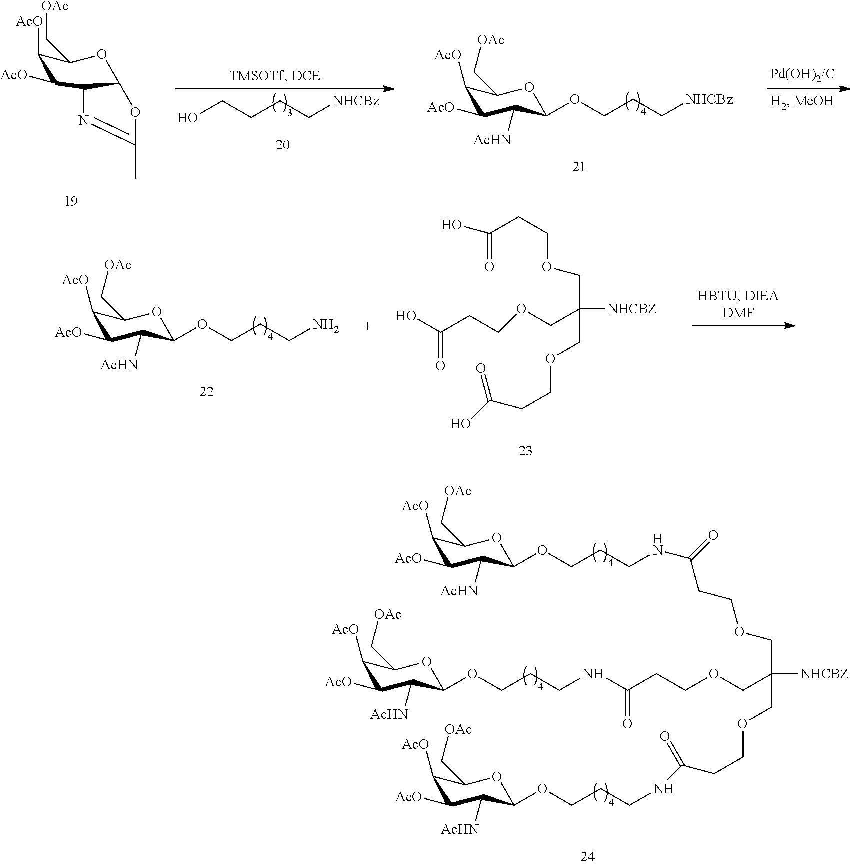

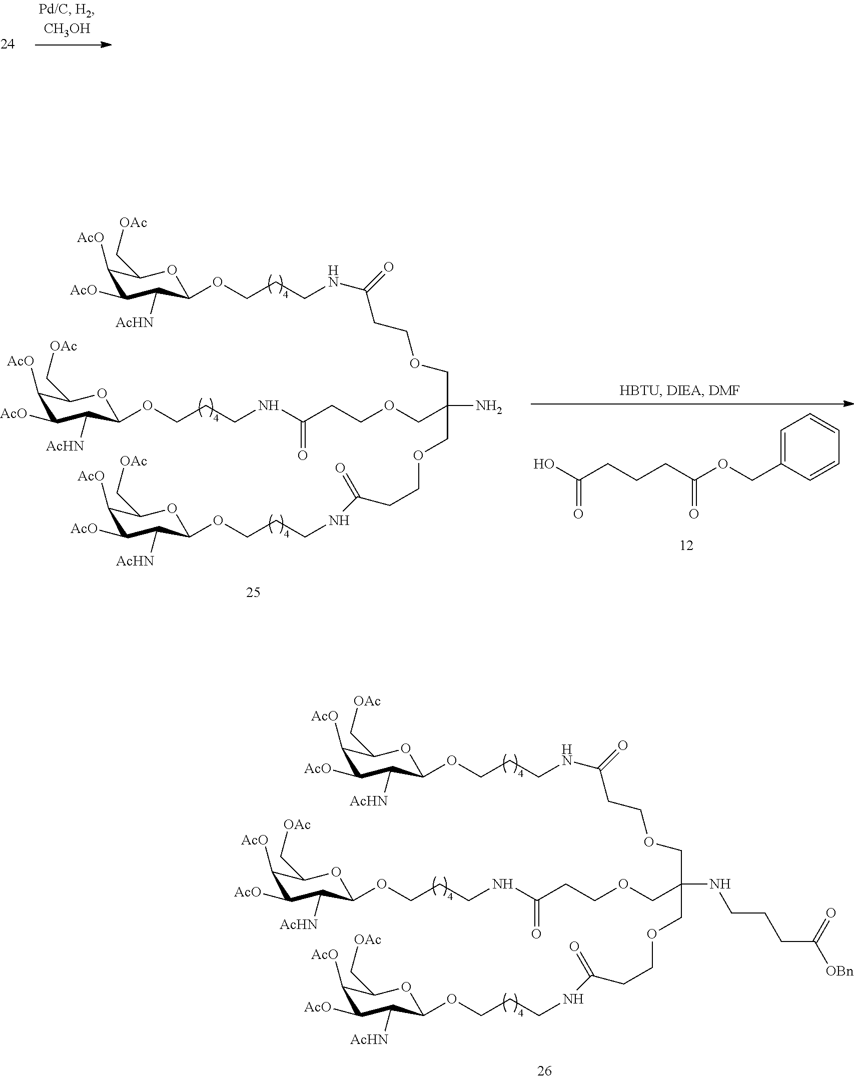

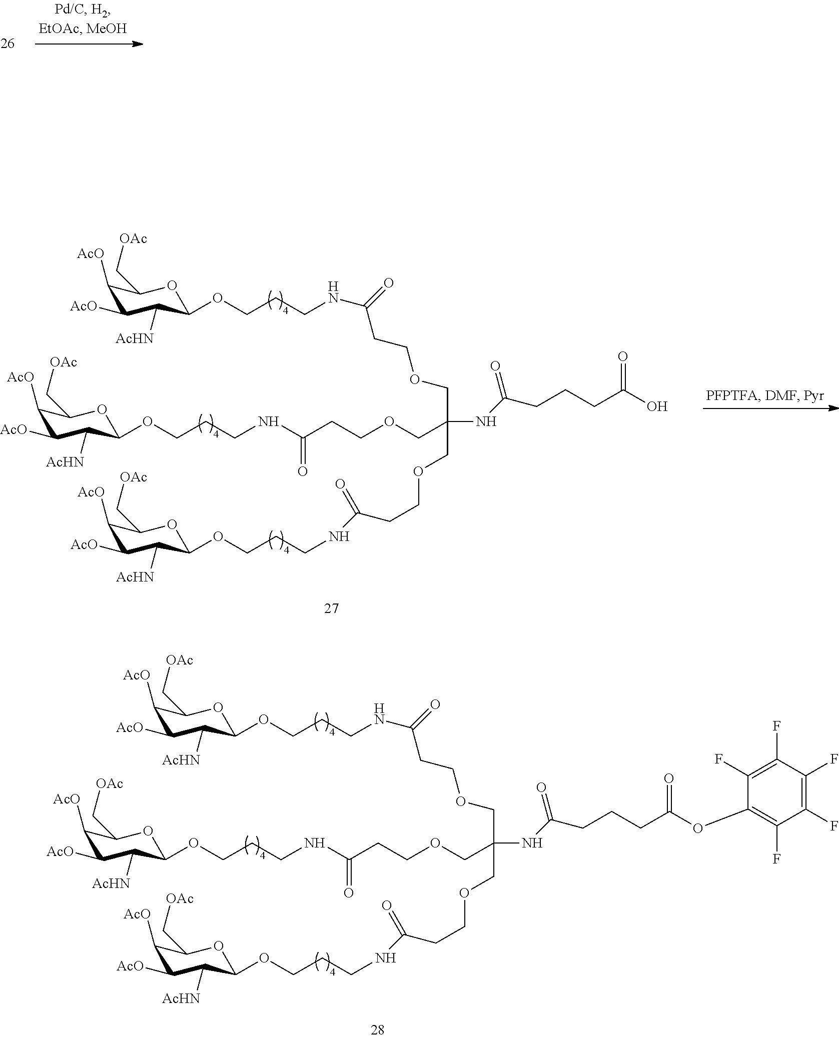

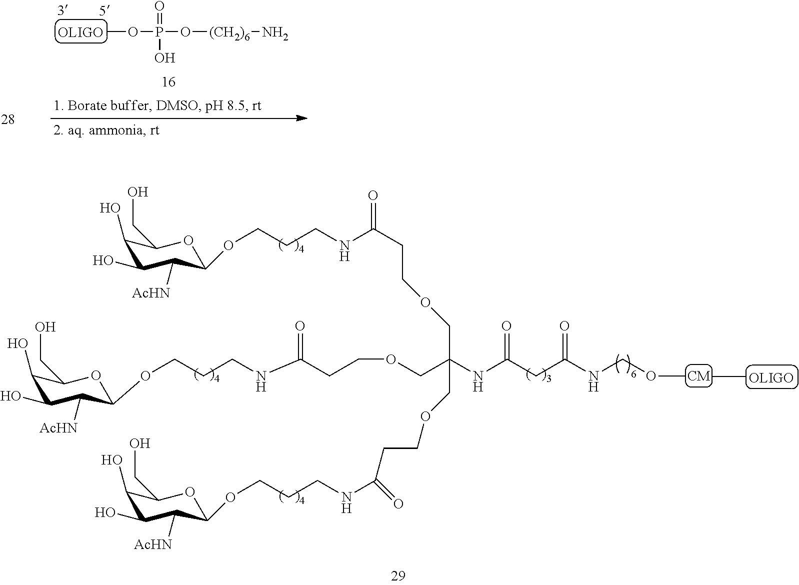

Compound 21 was synthesized following the procedure described in the literature (J. Med. Chem. 2004, 47, 5798-5808). The remaining reactions were performed as shown via standard organic chemistry methods, and oligomeric compound 29, comprising a GalNAc₃-7 conjugate group, is prepared using the general procedures illustrated in Example 1. The GalNAc₃ cluster portion of the conjugate group GalNAc₃-7 (GalNAc₃-7ₐ) can be combined with any cleavable moiety to provide a variety of conjugate groups. The structure of GalNAc₃-7 (GalNAc₃-7ₐ-CM-) is shown below:

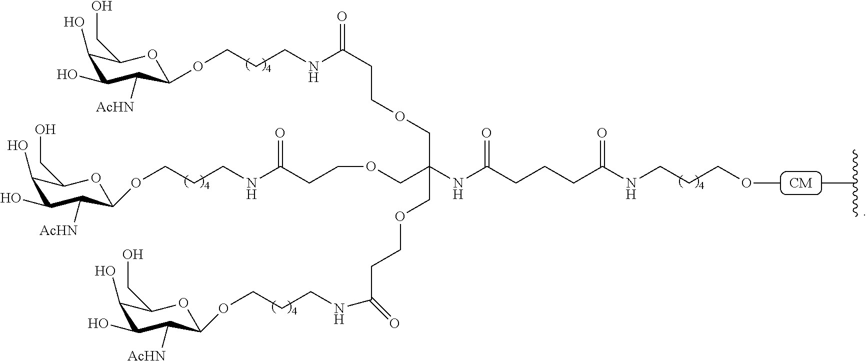

### Example 3: General Method for the Preparation of an Oligomeric Compound Comprising a GalNAc₃-10 Conjugate at the 5′ Terminus

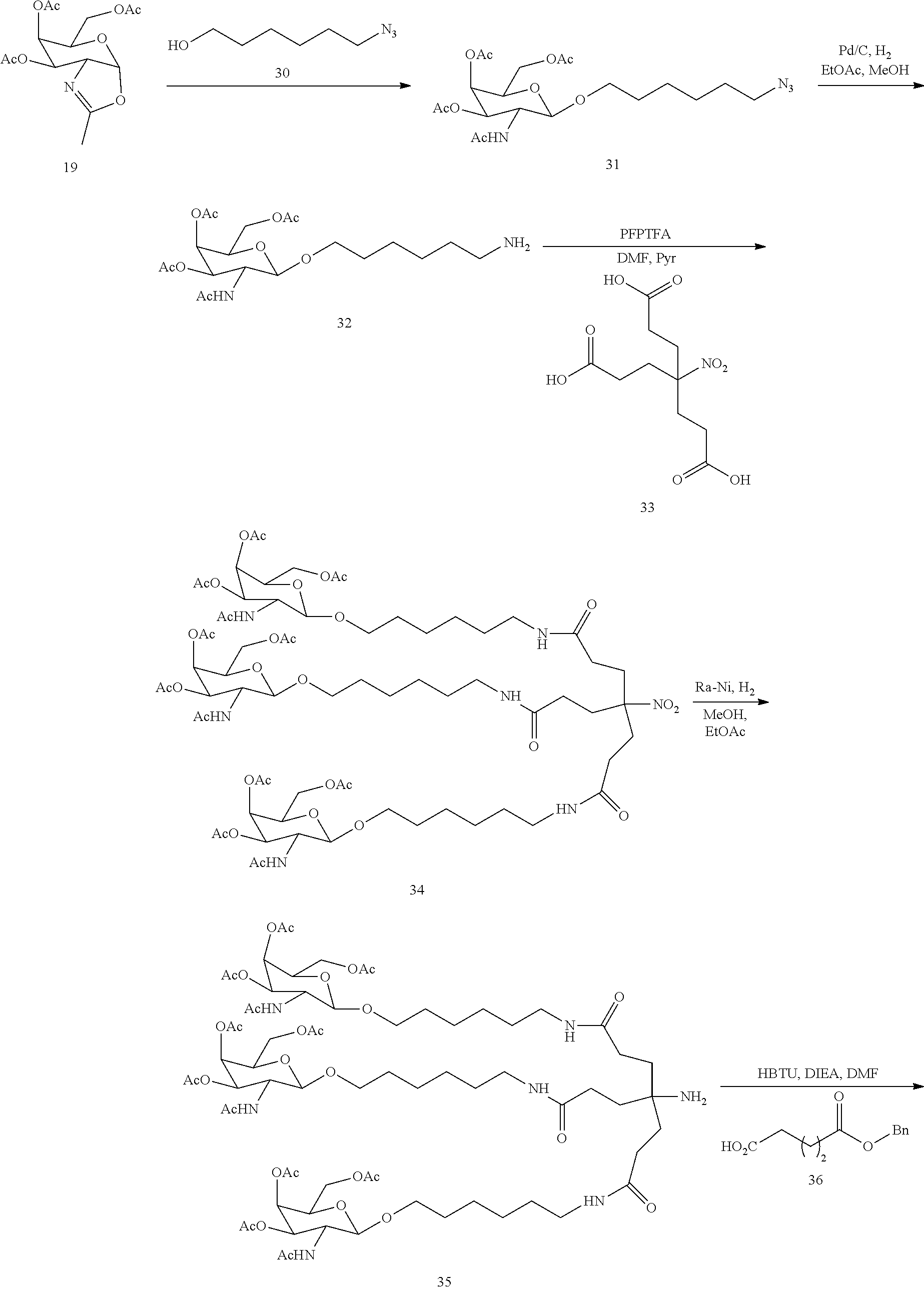

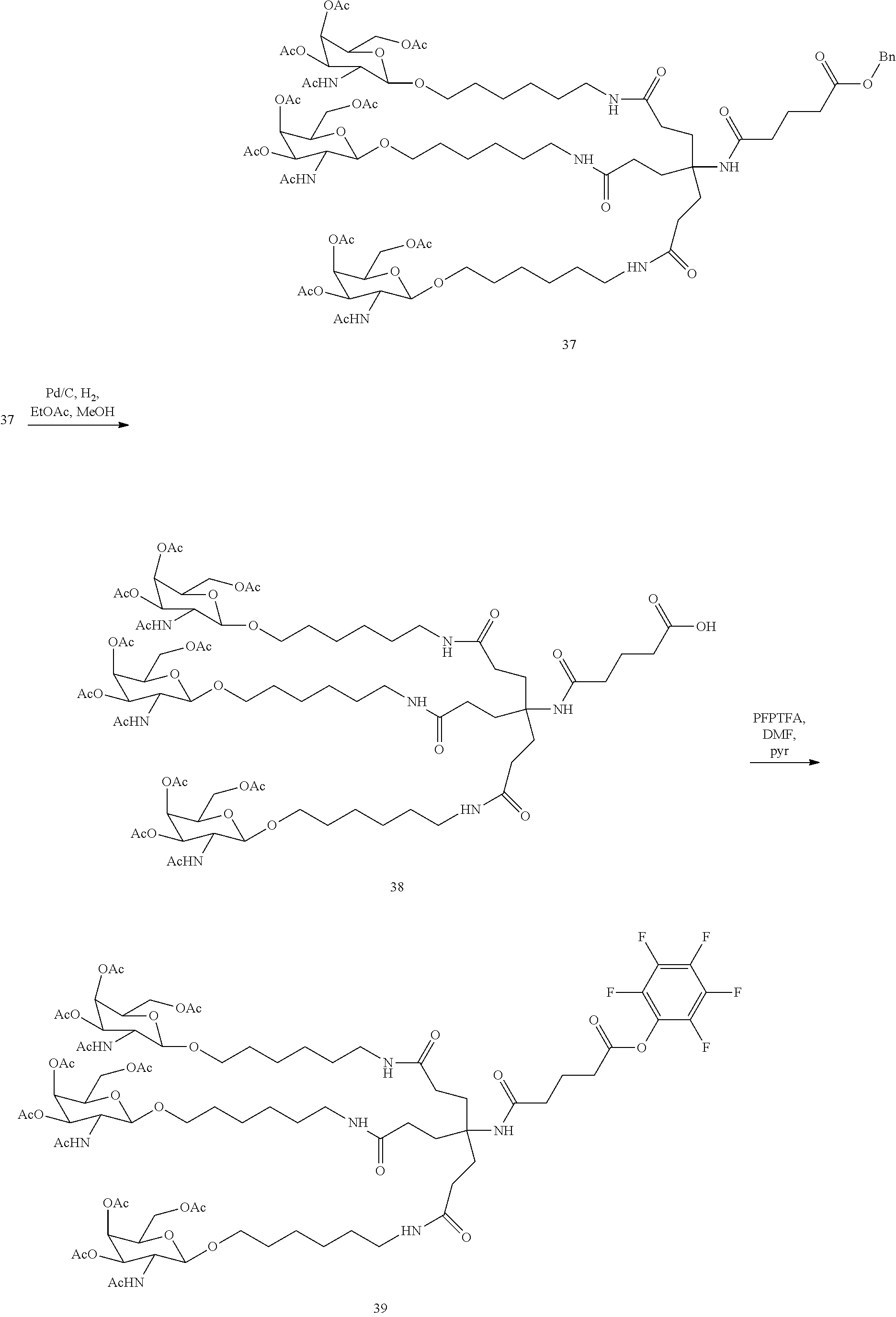

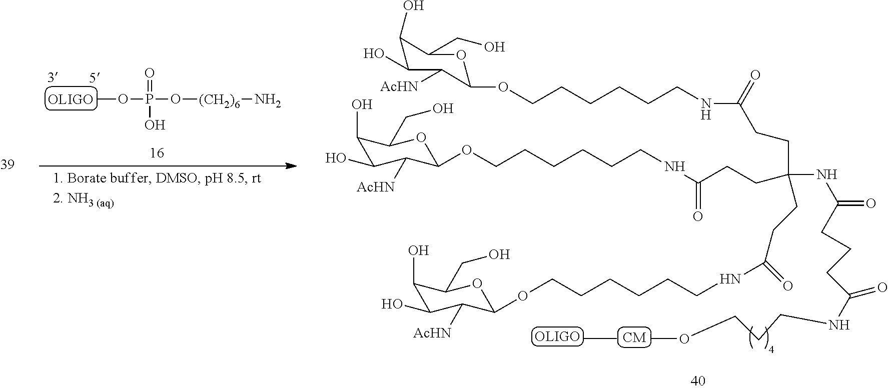

Oligomeric compound 40 comprises a GalNAc₃-10 conjugate. The GalNAc₃ cluster portion of the conjugate group GalNAc₃-10 (GalNAc₃-10ₐ) can be combined with any cleavable moiety to provide a variety of conjugate groups. The structure of GalNAc₃-10 (GalNAc₃-10ₐ-CM-) is shown below:

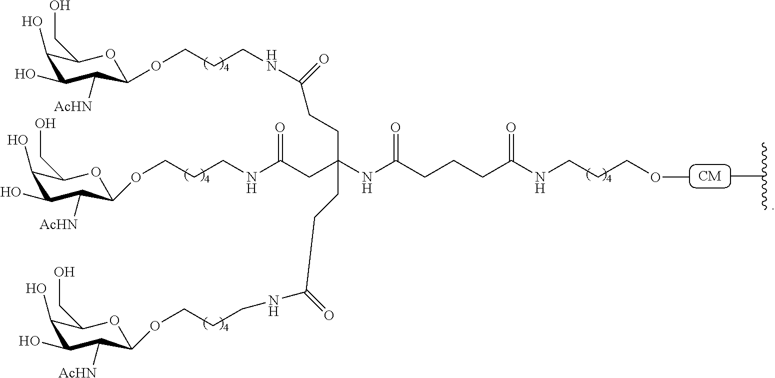

### Example 4: General Method for the Preparation of an Oligomeric Compound Comprising a GalNAc₃-13 Conjugate at the 5′ Terminus

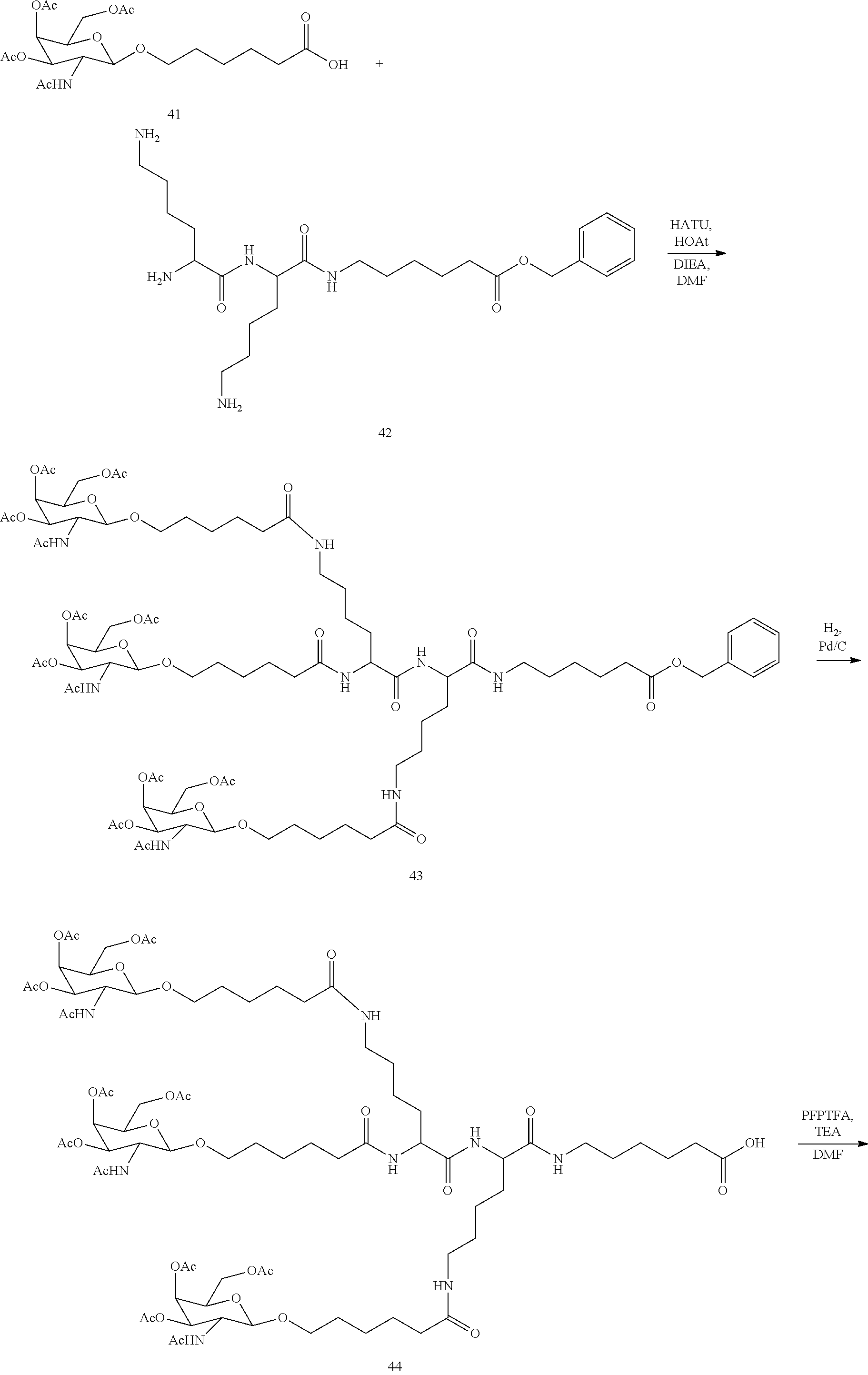

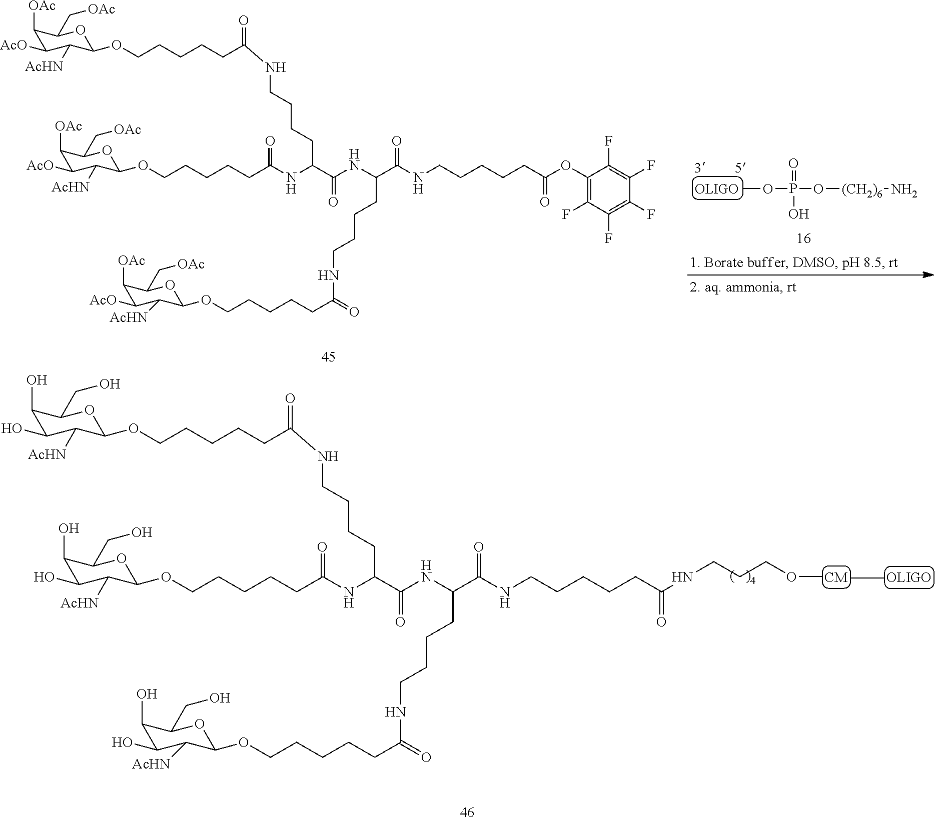

Oligomeric compound 46 comprises a GalNAc₃-13 conjugate group. The GalNAc₃ cluster portion of the conjugate group GalNAc₃-13 (GalNAc₃-13ₐ) can be combined with any cleavable moiety to provide a variety of conjugate groups. The structure of GalNAc₃-13 (GalNAc₃-13ₐ-CM-) is shown below:

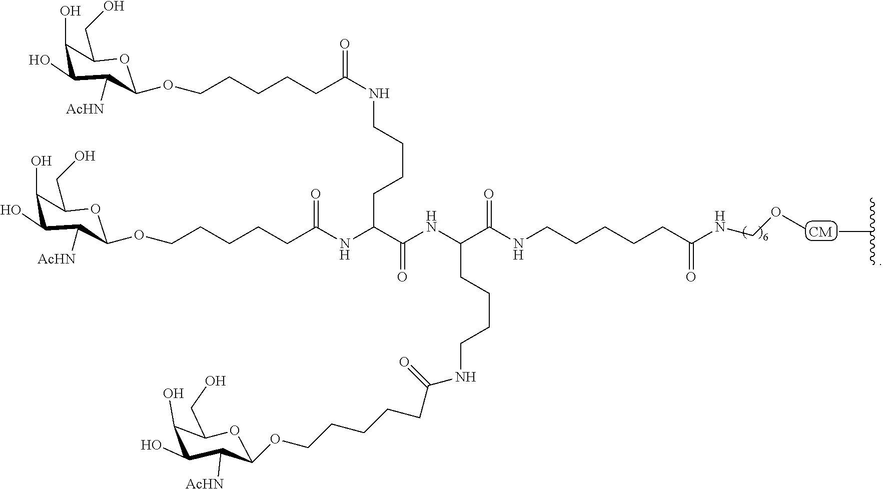

### Example 5: General Method for the Preparation of an Oligomeric Compound Comprising a GalNAc₃-19 Conjugate at the 3′ Terminus

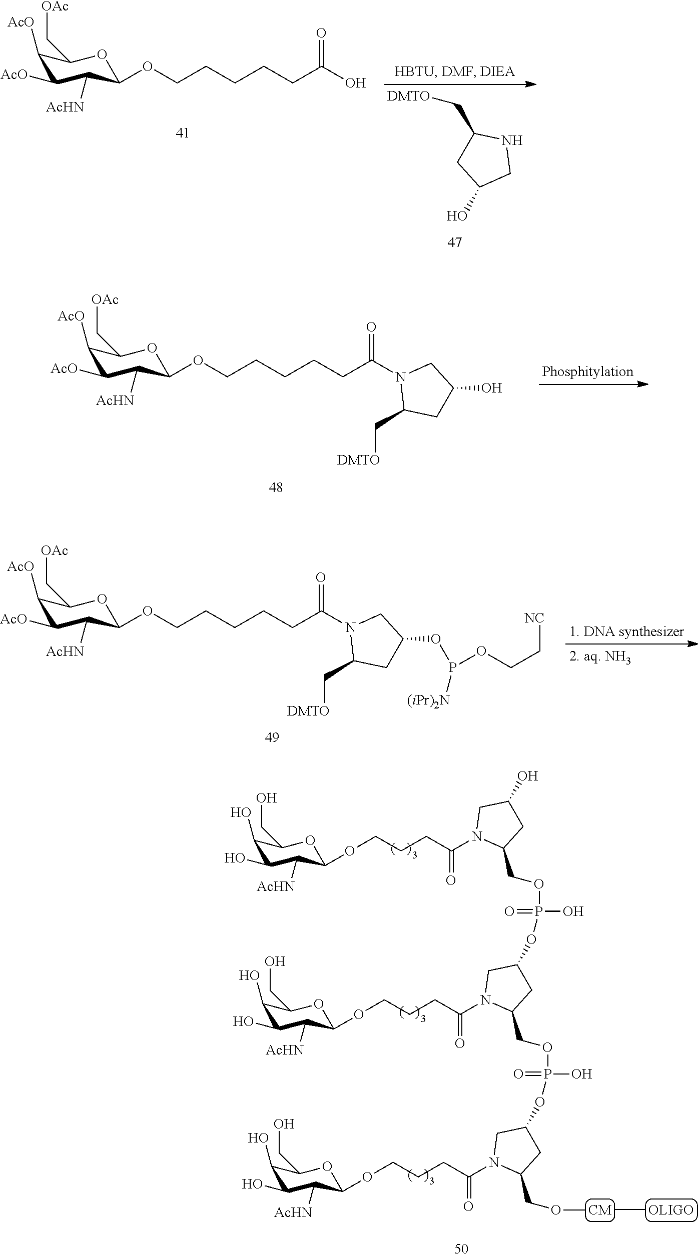

Oligomeric compound 50 comprises a GalNAc₃-19 conjugate group. The GalNAc₃ cluster portion of the conjugate group GalNAc₃-19 (GalNAc₃-19ₐ) can be combined with any cleavable moiety to provide a variety of conjugate groups. The structure of GalNAc₃-19 (GalNAc₃-19ₐ-CM-) is shown below:

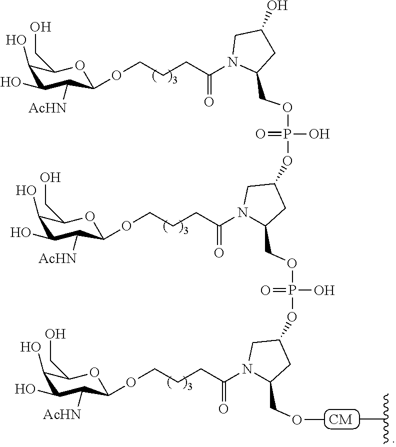

### Example 6: General Method for the Preparation of an Oligomeric Compound Comprising a GalNAc₃-1 Conjugate at the 3′ Terminus

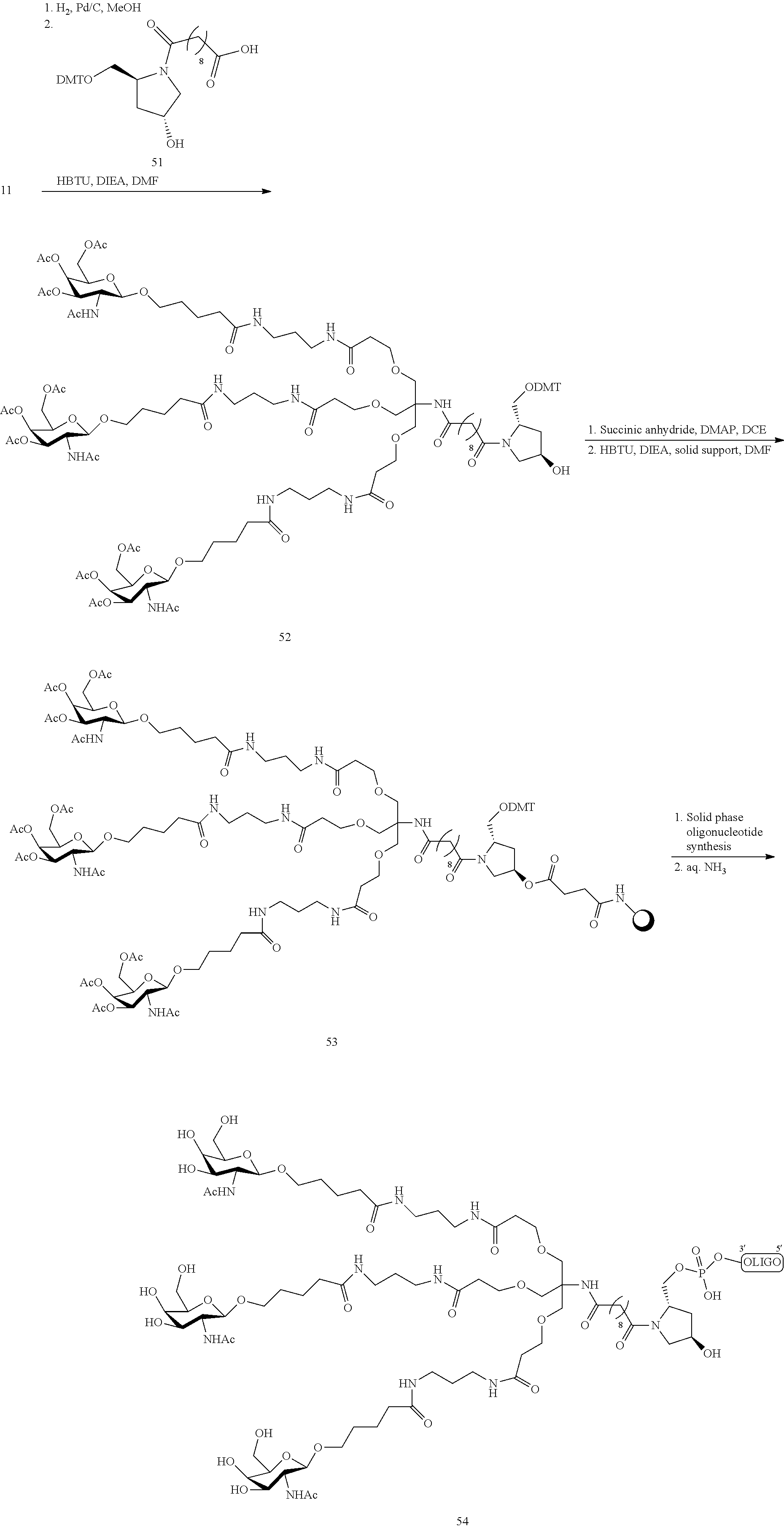

Oligomeric compound 54 comprises a GalNAc₃-1 conjugate group. The GalNAc₃ cluster portion of the conjugate group GalNAc₃-1 (GalNAc₃-1ₐ) can be combined with any cleavable moiety to provide a variety of conjugate groups. The structure of GalNAc₃-1 (GalNAc₃-1ₐ-CM-) is shown below:

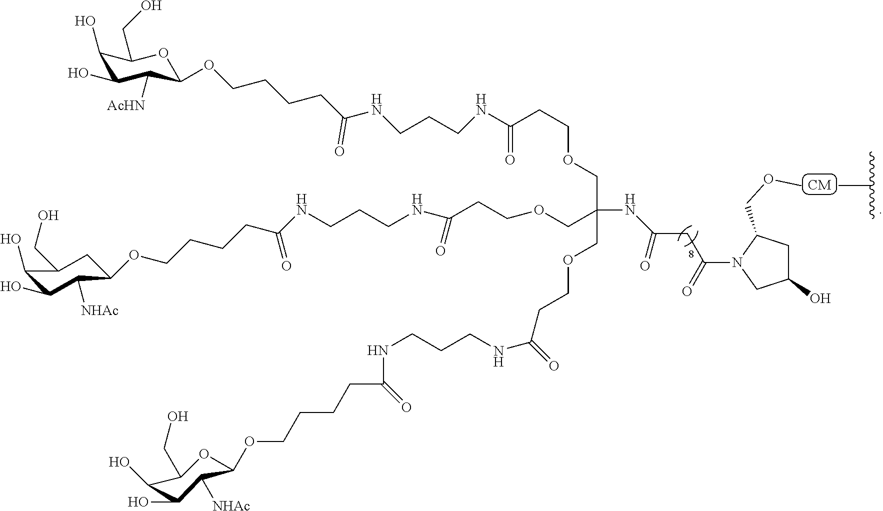

### Example 7: Stability of an Antisense Oligonucleotide Comprising a GalNAc Cluster in Rat Jejunum

ISIS 656172, an antisense oligonucleotide comprising a GalNAc₃-1 cluster and targeting mouse Factor XI, was tested in a stability study in rat jejunum. ISIS 656172 is a gapmer comprising 2′-methoxyethyl (MOE) modifications in the wings, and the cleavable moiety linking the 3′-GalNAc₃-1 cluster to the oligonucleotide is a phosphodiester linked deoxyadenosine. The sequence of ISIS 656172 is 5′-TGGTAATCCACTTTCAGAGGA-3′ (SEQ ID NO: 142), the cytosines are 5-methylcytosines, and the internucleoside linkages are phosphorothioate except for the phosphodiester linkage between the guanosine and the deoxyadenosine at the 3′-end.

Male Sprague Dawley rats were fasted overnight, then anesthetized with isoflurance. A ten centimeter segment of each rat's mid-jejunum was exposed and tied with suture thread to isolate it from the rest of the jejunum. Each segment was injected with 0.25 mL saline or 150 mg/mL sodium caprate (C₁₀) with or without ISIS 656172 at one of the dosages shown in Table 22 below. Each treatment group consisted of 1 animal. After a one hour incubation, the jejunum segments were rinsed with saline containing an internal standard oligonucleotide, and the segment contents were collected and analyzed by HPLC-MS. The amounts of ISIS 656172 and the “parent oligonucleotide” that does not comprise a GalNAc cluster were measured relative to the internal standard (IS). Additional ISIS 656172 degradation products were measured and used to determine the percentage of recovered oligonucleotide that was intact ISIS 656172. The results are shown in Table 22.

As illustrated in Table 22, ISIS 656172, an antisense oligonucleotide comprising a GalNAc cluster, was stable in the rat jejunum for a duration of one hour.

TABLE 22
Ratios of antisense oligonucleotide:internal
standard (IS) recovered from rat jejunum

| | ISIS | | | % of recovered |
|---|---|---|---|---|
| | 656172 | | Parent | oligonucleotide |
| | dosage | ISIS | oligo- | that was intact |
| Vehicle | (mg/mL) | 656172:IS | nucleotide:IS | ISIS 656172 |
| Saline | 1 | 0.185 | 0.009 | 95.3 |
| C10 | 1 | 0.207 | 0.010 | 95.5 |
| Saline | 10 | 1.24 | 0.055 | 95.8 |
| C10 | 10 | 1.35 | 0.065 | 95.4. |

### Example 8: Stability of Antisense Oligonucleotides Comprising Various GalNAc Clusters in Rat Jejunum

The oligonucleotides listed in Table 23 below were tested in a stability study in rat jejunum. ISIS 3521 is known to be unstable in the jejunum and was included as a control. If present, the GalNAc cluster and cleavable moiety is bolded in each sequence.

TABLE 23

| | | SEQ |
|---|---|---|
| ISIS | | ID |
| No. | Sequences (5′to 3′) | No. |
| 3521 | G_{ds}T_{ds}T_{ds}C_{ds}T_{ds}C_{ds}G_{ds}C_{ds}T_{ds}G_{ds}G_{ds}T_{ds}G_{ds}A_{ds}G_{ds}T_{ds} | 143 |
| | T_{ds}T_{ds}C_{ds}A_{d} | |
| 440670 | ^{m}CₑₛAₑₛGₑₛ^{m}CₑₛTₑₛT_{ds}T_{ds}A_{ds}T_{ds}T_{ds}A_{ds}G_{ds}G_{ds}G_{ds}A_{ds} | 144 |
| | ^{m}CₑₛAₑₛGₑₛ^{m}CₑₛAₑ | |
| 661180 | ^{m}CₑₛAₑₛGₑₛ^{m}CₑₛTₑₛT_{ds}T_{ds}A_{ds}T_{ds}T_{ds}A_{ds}G_{ds}G_{ds}G_{ds}A_{ds} | 145 |
| | ^{m}CₑₛAₑₛGₑₛ^{m}CₑₛAₑₒA_{do′}-GalNAc₃-1ₐ | |
| 680771 | GalNAc₃-3_{a-o′}^{m}CₑₛAₑₛGₑₛ^{m}CₑₛTₑₛT_{ds}T_{ds}A_{ds}T_{ds}T_{ds} | 144 |
| | A_{ds}G_{ds}G_{ds}G_{ds}A_{ds}^{m}CₑₛAₑₛGₑₛ^{m}CₑₛAₑ | |
| 680772 | GalNAc₃-7_{a-o′}^{m}CₑₛAₑₛGₑₛ^{m}CₑₛTₑₛT_{ds}T_{ds}A_{ds}T_{ds}T_{ds} | 144 |
| | A_{ds}G_{ds}G_{ds}G_{ds}A_{ds}^{m}CₑₛAₑₛGₑₛ^{m}CₑₛAₑ | |
| 680773 | GalNAc_{₃}-10_{a-o′}^{m}CₑₛAₑₛGₑₛ^{m}CₑₛTₑₛT_{ds}T_{ds}A_{ds}T_{ds}T_{ds} | 144 |
| | A_{ds}G_{ds}G_{ds}G_{ds}A_{ds}^{m}CₑₛAₑₛGₑₛ^{m}CₑₛAₑ | |
| 680774 | GalNAc₃-13_{a-o′}^{m}CₑₛAₑₛGₑₛ^{m}CₑₛTₑₛT_{ds}T_{ds}A_{ds}T_{ds}T_{ds} | 144 |
| | A_{ds}G_{ds}G_{ds}G_{ds}A_{ds}^{m}CₑₛAₑₛGₑₛ^{m}CₑₛAₑ | |

In the sequences in all tables, capital letters indicate the nucleobase for each nucleoside and ^{m}C indicates a 5-methylcytosine. Subscripts: “e” indicates a 2′-MOE modified nucleoside; “d” indicates a β-D-2′-deoxyribonucleoside; “s” indicates a phosphorothioate internucleoside linkage (PS); “o” indicates a phosphodiester internucleoside linkage (PO); and “o′” indicates —O—P(═O)(OH)—.

Male Sprague Dawley rats were treated as described in Example 7, and the results are shown in Table 24. The treatment group that received ISIS 3521 consisted of two animals. The “full oligonucleotide” comprises the intact GalNAc cluster (if present), and the “parent oligonucleotide” is the intact oligonucleotide that does not comprise a GalNAc cluster. For ISIS 3521 and 440670, which do not comprise a GalNAc cluster, the “full” and “parent” oligonucleotides are the same compounds.

TABLE 24
Ratios of antisense oligonucleotide:internal
standard (IS) recovered from rat jejunum

| | | | | | % of recovered |
|---|---|---|---|---|---|
| | | | Full | Parent | oligonucleotide |
| | | | oligo- | oligo- | that was intact, |
| | ISIS | Dosage | nucleo- | nucleo- | full oligonu- |
| Vehicle | No. | (mg/mL) | tide:IS | tide:IS | cleotide |
| C10 | 3521 | 10 | 2.59 | n/a | 76.0 |
| C10 | 440670 | 10 | 0.74 | n/a | 100.0 |
| C10 | 661180 | 10 | 2.10 | 0.00 | 100.0 |
| C10 | 680771 | 10 | 0.96 | 0.00 | 100.0 |
| C10 | 680772 | 10 | 1.14 | 0.00 | 100.0 |
| C10 | 680773 | 10 | 1.43 | 0.00 | 100.0 |
| C10 | 680774 | 10 | 1.44 | 0.01 | 93.4. |

### Example 9: Bioavailability of Antisense Oligonucleotides Comprising Various GalNAc Clusters Administered Intrajejunally

Antisense oligonucleotides targeting rat metastasis associated lung adenocarcinoma transcript 1 (MALAT-1) are tested in a bioavailability study in rat. The oligonucleotides are gapmers that are 16 nucleotides in length, comprising cEt modifications in the wings that are each three nucleotides in length. Each pair of oligonucleotides contains the same sequence, the “parent” does not comprise a GalNAc cluster, and the second oligonucleotide comprises a GalNAc₃-7 cluster attached to the 5′-end of the oligonucleotide via a cleavable phosphodiester linkage. For example, the oligonucleotides in Table 25 will be tested for bioavailability in rat.

TABLE 25
Antisense oligonucleotides for use in
bioavailability testing in rat

| | | SEQ |
|---|---|---|
| ISIS | | ID |
| No. | Sequences (5′ to 3′) | No. |
| 556116 | Aₖₛ^{m}Cₖₛ^{m}CₖₛA_{ds}T_{ds}G_{ds}A_{ds}T_{ds}A_{ds}^{m}C_{ds}^{m}C_{ds}A_{ds}^{m}C_{ds}TₖₛTₖₛ | 146 |
| | Tₖ | |
| 704361 | GalNAc₃-7_{a-o′} Aₖₛ^{m}Cₖₛ^{m}CₖₛA_{ds}T_{ds}G_{ds}A_{ds}T_{ds} | 146 |
| | A_{ds}^{m}C_{ds}^{m}C_{ds}A_{ds}^{m}C_{ds}TₖₛTₖₛTₖ | |

Subscript “k” indicates a cEt modified nucleoside. See Table 24 for list of other abbreviations.

The oligonucleotides are administered to Sprague Dawley rats intrajejunally. After the animals are sacrificed, MALAT-1 mRNA levels in the liver are analyzed by RT-PCR.

### Example 10: General Method for the Preparation of an Oligomeric Compound Comprising a GalNAc₂-24 Conjugate at the 5′ Terminus

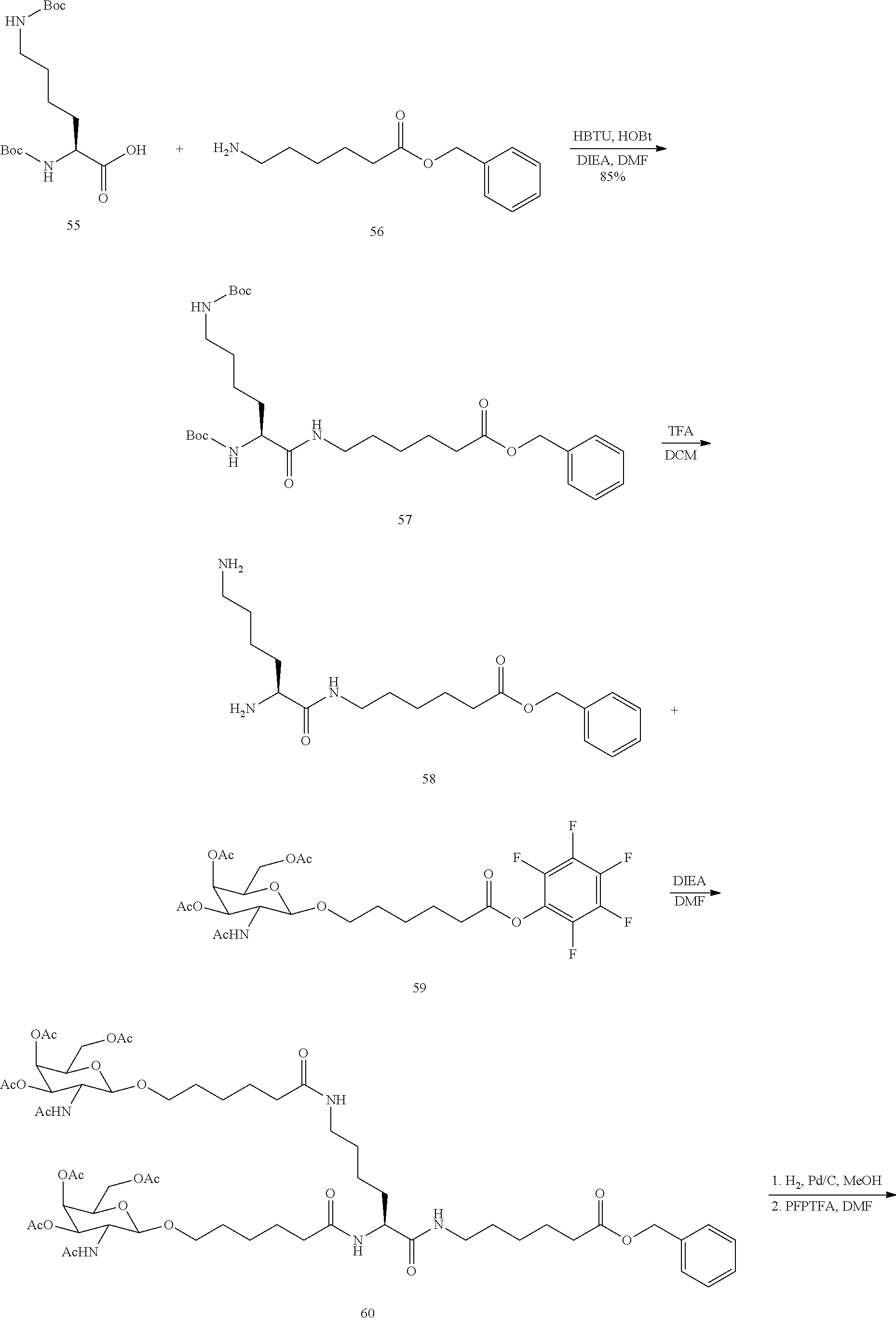

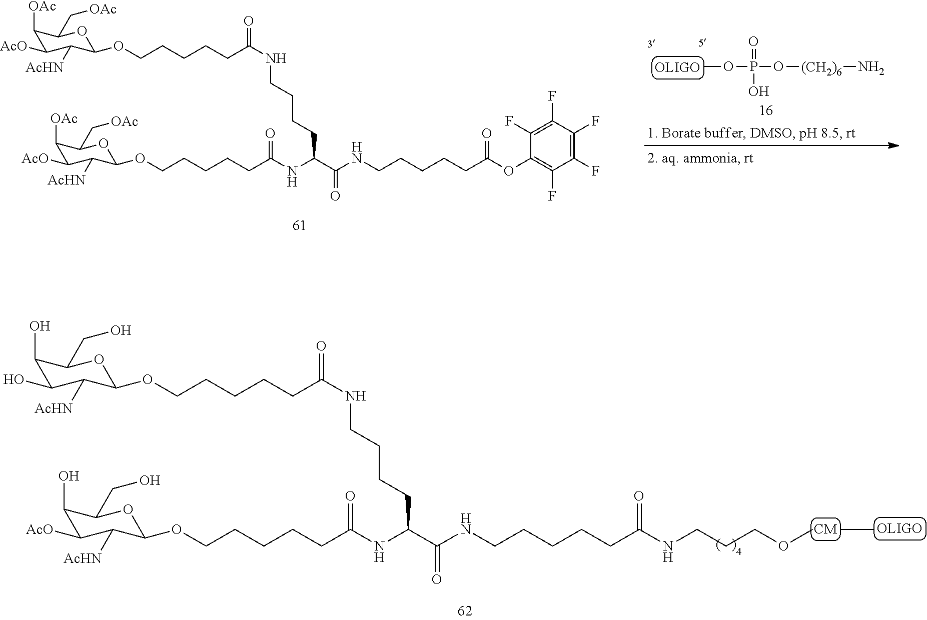

Compound 55 is commercially available, and compound 56 was synthesized following the procedure described in the literature (J. Am. Chem. Soc. 2011, 133, 958-963). Compound 55 (1 g, 2.89 mmol), HBTU (0.39 g, 2.89 mmol), and HOBt (1.64 g, 4.33 mmol) were dissolved in DMF (10 mL. and N,N-diisopropylethylamine (1.75 mL, 10.1 mmol) were added. After about 5 min, aminohexanoic acid benzyl ester (1.36 g, 3.46 mmol) was added to the reaction. After 3 h, the reaction mixture was poured into 100 mL of 1 M NaHSO4 and extracted with 2×50 mL ethyl acetate. Organic layers were combined and washed with 3×40 mL sat NaHCO₃ and 2×brine, dried with Na₂SO₄, filtered and concentrated. The product was purified by silica gel column chromatography (DCM:EA:Hex, 1:1:1) to yield compound 57. LCMS and NMR were consistent with the structure. Compound 57 (1.34 g, 2.438 mmol) was dissolved in dichloromethane (10 mL) and trifluoracetic acid (10 mL) was added. After stirring at room temperature for 2 h, the reaction mixture was concentrated under reduced pressure and co-evaporated with toluene (3×10 mL). The residue was dried under reduced pressure to yield compound 58 as the trifuloracetate salt. Compound 59 (3.39 g, 5.40 mmol) was dissolved in DMF (3 mL). A solution of compound 58 (1.3 g, 2.25 mmol) was dissolved in DMF (3 mL) and N,N-diisopropylethylamine (1.55 mL) was added. The reaction was stirred at room temperature for 30 minutes, then poured into water (80 mL) and the aqueous layer was extracted with EtOAc (2×100 mL). The organic phase was separated and washed with sat. aqueous NaHCO₃ (3×80 mL), 1 M NaHSO4 (3×80 mL) and brine (2×80 mL), then dried (Na₂SO₄), filtered, and concentrated. The residue was purified by silica gel column chromatography to yield compound 60. LCMS and NMR were consistent with the structure.

Compound 60 (0.59 g, 0.48 mmol) was dissolved in methanol (2.2 mL) and ethyl acetate (2.2 mL). Palladium on carbon (10 wt % Pd/C, wet, 0.07 g) was added, and the reaction mixture was stirred under hydrogen atmosphere for 3 h. The reaction mixture was filtered through a pad of Celite and concentrated to yield the carboxylic acid. The carboxylic acid (1.32 g, 1.15 mmol, cluster free acid) was dissolved in DMF (3.2 mL). To this N,N-diisopropylehtylamine (0.3 mL, 1.73 mmol) and PFPTFA (0.30 mL, 1.73 mmol) were added. After 30 min stirring at room temperature the reaction mixture was poured into water (40 mL) and extracted with EtOAc (2×50 mL). A standard work-up was completed as described above to yield compound 61. LCMS and NMR were consistent with the structure. Oligomeric compound 62 comprises a GalNAc₂-24 conjugate group. The GalNAc₂ cluster portion (GalNAc₂-24ₐ) of the conjugate group GalNAc₂-24 can be combined with any cleavable moiety present on the oligonucleotide to provide a variety of conjugate groups. The structure of GalNAc₂-24 (GalNAc₂-24ₐ-CM) is shown below:

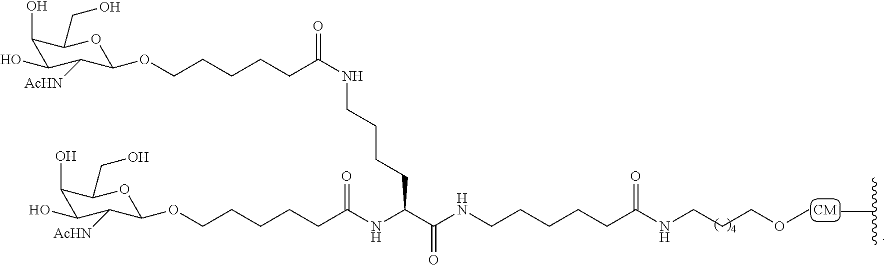

### Example 11: General Methods for the Preparation of Oligomeric Compounds Comprising a GalNAc₁-25 Conjugate at the 5′ Terminus

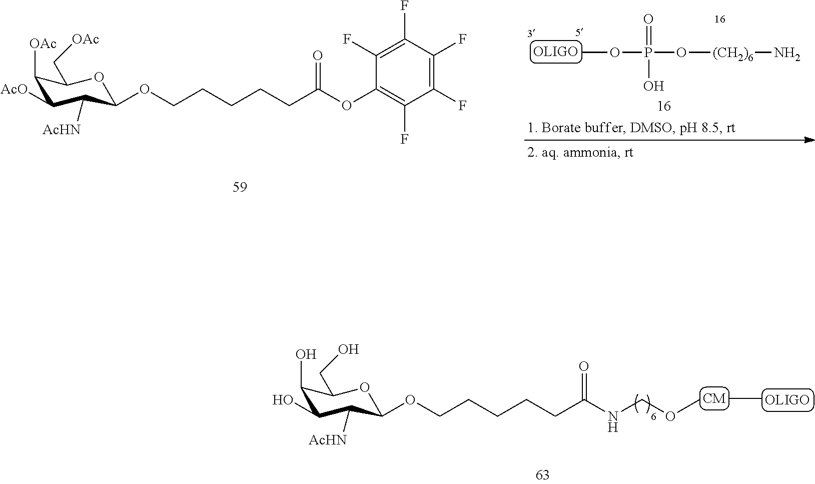

Oligonucleotide 63 comprises a GalNAc₁-25 conjugate group. Alternatively, oligonucleotide 63 was synthesized using the scheme shown below.

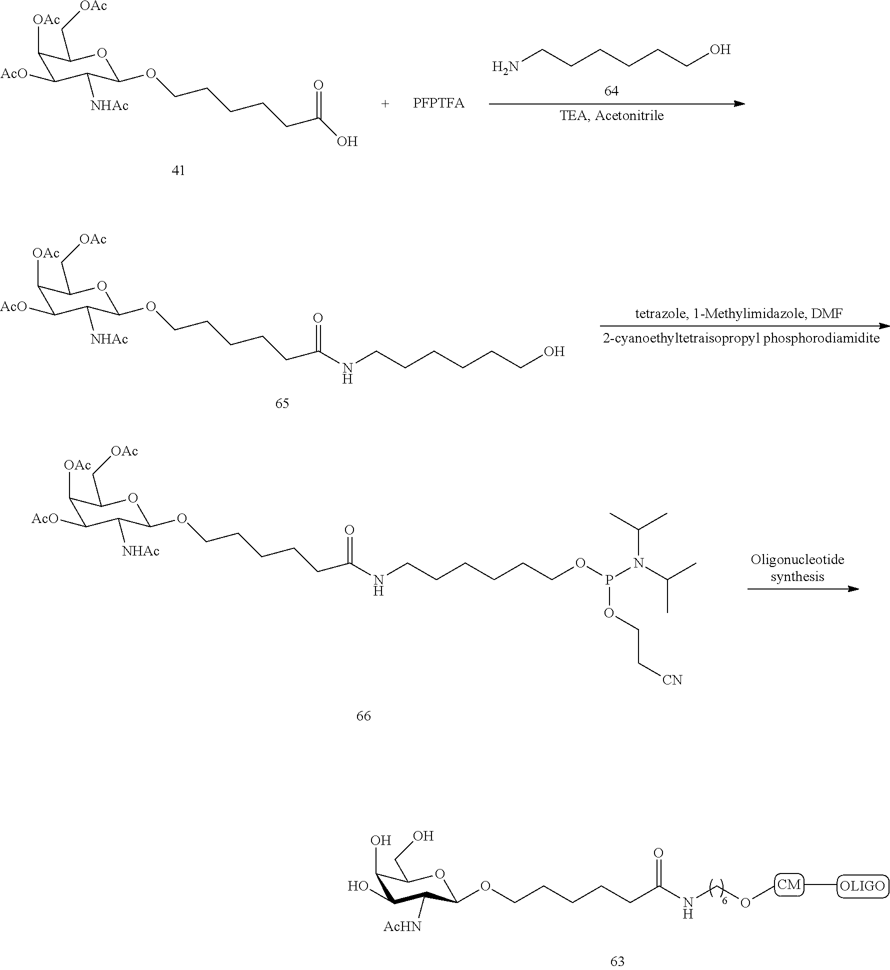

The GalNAc₁ cluster portion (GalNAc₁-25ₐ) of the conjugate group GalNAc₁-25 can be combined with any cleavable moiety present on the oligonucleotide to provide a variety of conjugate groups. The structure of GalNAc₁-25 (GalNAc₁-25ₐ-CM) is shown below:

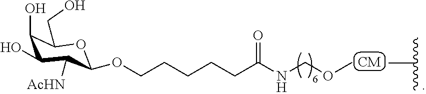

### Example 12: General Methods for the Preparation of Oligomeric Compounds Comprising a GalNAc₁-26 Conjugate at the 5′ Terminus or a GalNAc₁-27 Conjugate at the 3′ Terminus

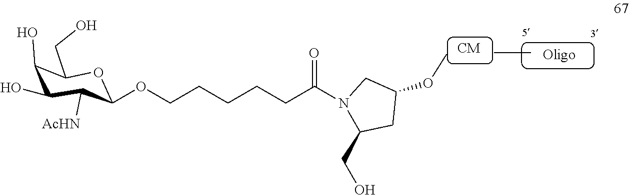

Oligonucleotide 67 is synthesized via coupling of compound 47 to acid 41 (see Example 5) using HBTU and DIEA in DMF. The resulting amide containing compound is phosphitylated, then added to the 5′-end of an oligonucleotide. The GalNAc₁ cluster portion (GalNAc₁-26ₐ) of the conjugate group GalNAc₁-26 can be combined with any cleavable moiety present on the oligonucleotide to provide a variety of conjugate groups. The structure of GalNAc₁-26 (GalNAc₁-26ₐ-CM) is shown below:

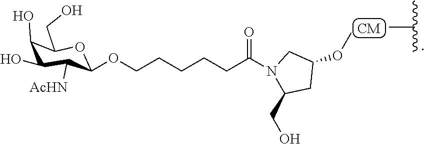

In order to add the GalNAc₁ conjugate group to the 3′-end of an oligonucleotide, the amide formed from the reaction of compounds 47 and 41 is added to a solid support. The oligonucleotide synthesis is then completed in order to form oligonucleotide 68, which comprises a GalNAc₁-27 conjugate group.

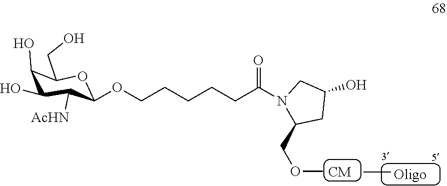

The GalNAc₁ cluster portion (GalNAc₁-27ₐ) of the conjugate group GalNAc₁-27 can be combined with any cleavable moiety present on the oligonucleotide to provide a variety of conjugate groups. The structure of GalNAc₁-27 (GalNAc₁-27ₐ-CM) is shown below:

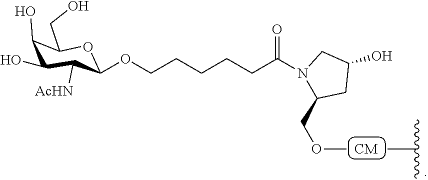

### Example 13: General Methods for the Preparation of Oligomeric Compounds Comprising a GalNAc₁-28 Conjugate at the 5′ Terminus or a GalNAc₁-29 Conjugate at the 3′ Terminus

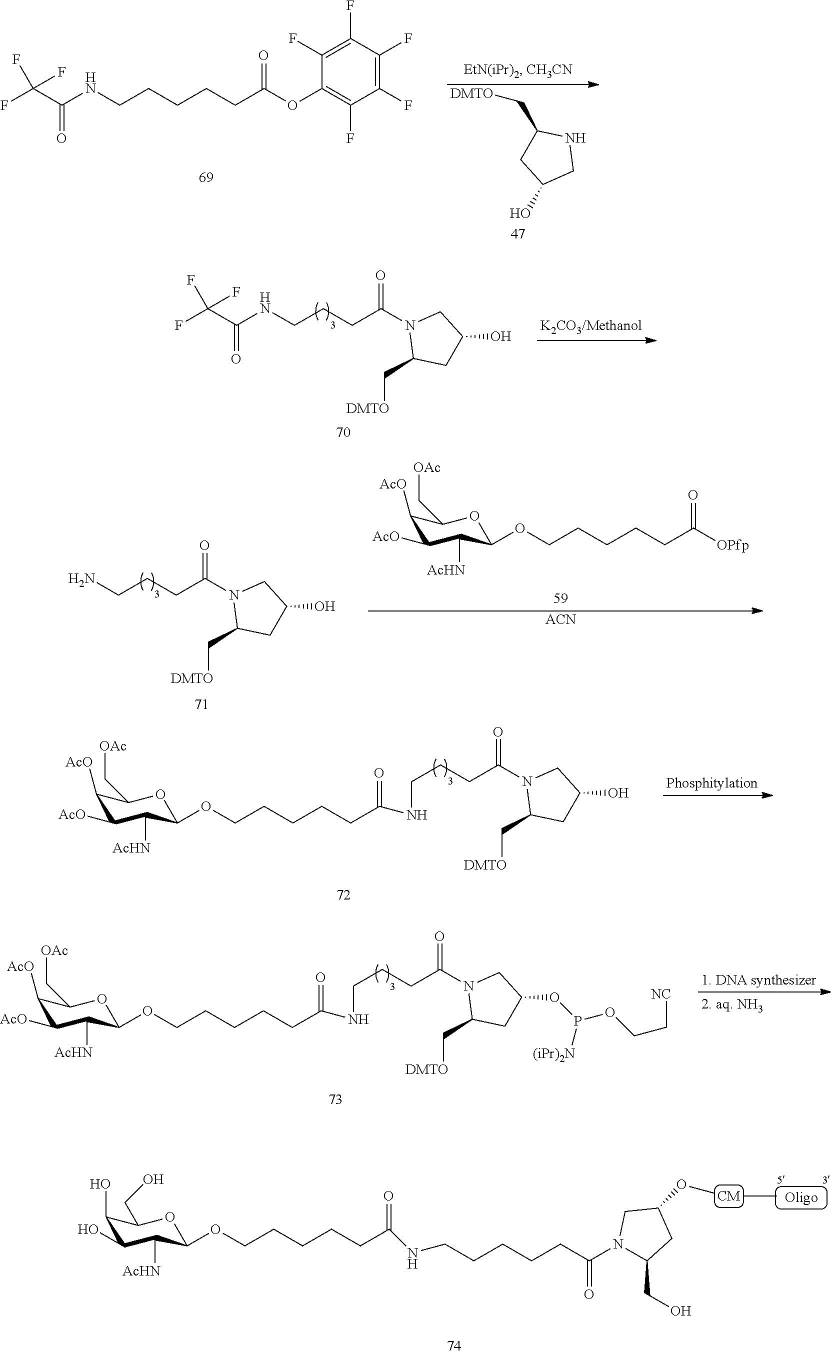

Oligonucleotide 74, which comprises a GalNAc₁-28 conjugate group, is synthesized by adding phosphoramidite 73 to the 5′-end of an oligonucleotide attached to a solid support. The GalNAc₁ cluster portion (GalNAc₁-28ₐ) of the conjugate group GalNAc₁-28 can be combined with any cleavable moiety present on the oligonucleotide to provide a variety of conjugate groups. The structure of GalNAc₁-28 (GalNAc₁-28ₐ-CM) is shown below:

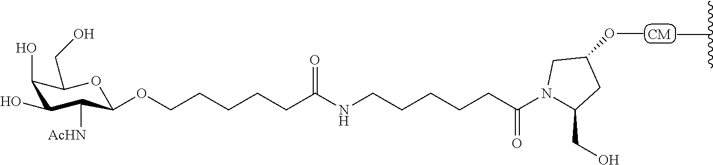

In order to add the GalNAc₁ conjugate group to the 3′-end of an oligonucleotide, compound 72 is added to a solid support, and oligonucleotide synthesis is then completed. The resulting oligonucleotide 75 comprises a GalNAc₁-29 conjugate group.

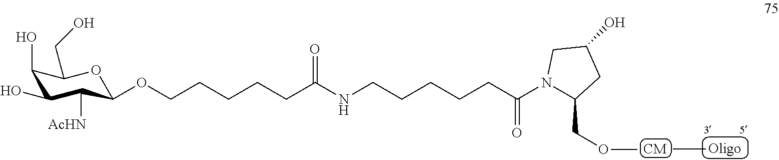

The GalNAc₁ cluster portion (GalNAc₁-29ₐ) of the conjugate group GalNAc₁-29 can be combined with any cleavable moiety present on the oligonucleotide to provide a variety of conjugate groups. The structure of GalNAc₁-29 (GalNAc₁-29ₐ-CM) is shown below:

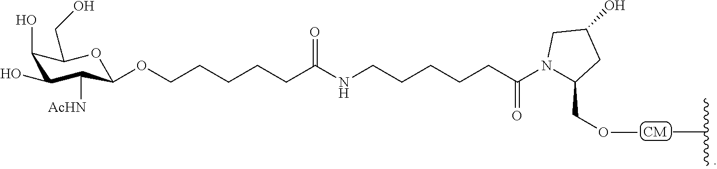

### Example 14: General Method for the Preparation of an Oligomeric Compound Comprising a GalNAc₁-30 Conjugate at the 5′ Terminus

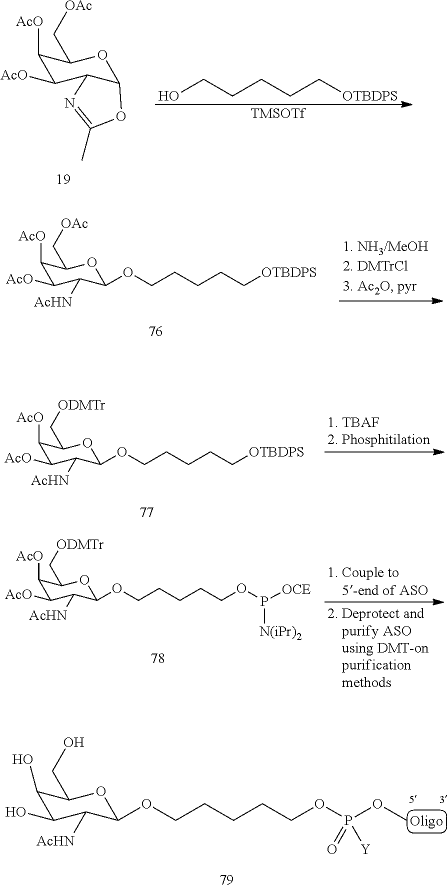

Oligonucleotide 79 comprises a GalNAc₁-30 conjugate group, wherein Y is selected from O, S, a substituted or unsubstituted C₁-C₁₀ alkyl, amino, substituted amino, azido, alkenyl or alkynyl. The GalNAc₁ cluster portion (GalNAc₁-30ₐ) of the conjugate group GalNAc₁-30 can be combined with any cleavable moiety to provide a variety of conjugate groups. In certain embodiments, Y is part of the cleavable moiety. In certain embodiments, Y is part of a stable moiety, and the cleavable moiety is present on the oligonucleotide. The structure of GalNAc₁-30ₐ is shown below:

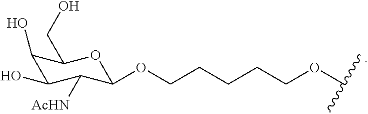 [SMILES: *OCCCCCOC1OC(CO)C(O)C(O)C1NC(C)=O]

### Example 15: General Methods for the Preparation of Oligomeric Compounds Comprising a GalNAc₂-31 Conjugate or a GalNAc₂-32 Conjugate at the 5′ Terminus

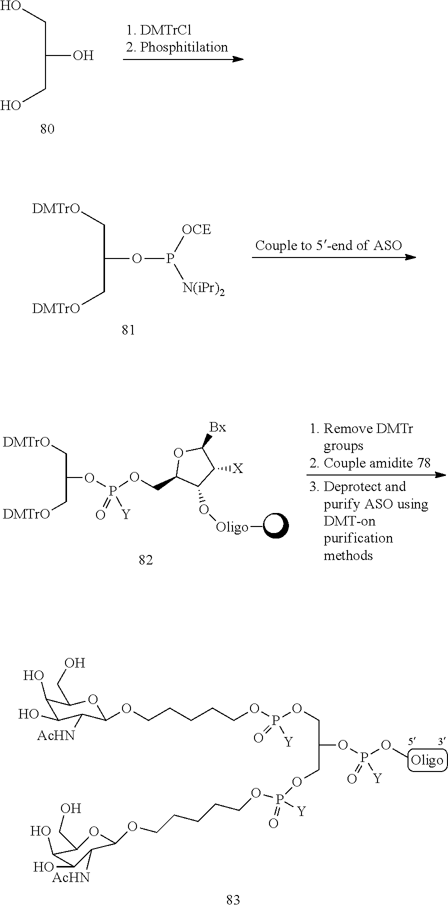

Oligonucleotide 83 comprises a GalNAc₂-31 conjugate group, wherein Y is selected from O, S, a substituted or unsubstituted C₁-C₁₀ alkyl, amino, substituted amino, azido, alkenyl or alkynyl. The GalNAc₂ cluster portion (GalNAc₂-31ₐ) of the conjugate group GalNAc₂-31 can be combined with any cleavable moiety to provide a variety of conjugate groups. In certain embodiments, the Y-containing group directly adjacent to the 5′-end of the oligonucleotide is part of the cleavable moiety. In certain embodiments, the Y-containing group directly adjacent to the 5′-end of the oligonucleotide is part of a stable moiety, and the cleavable moiety is present on the oligonucleotide. The structure of GalNAc₂-31ₐ is shown below:

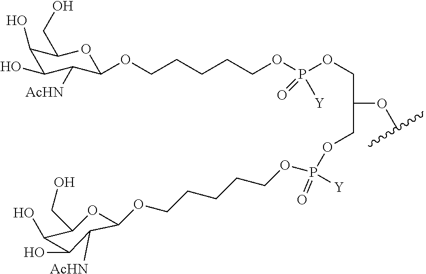

The synthesis of an oligonucleotide comprising a GalNAc₂-32 conjugate is shown below.

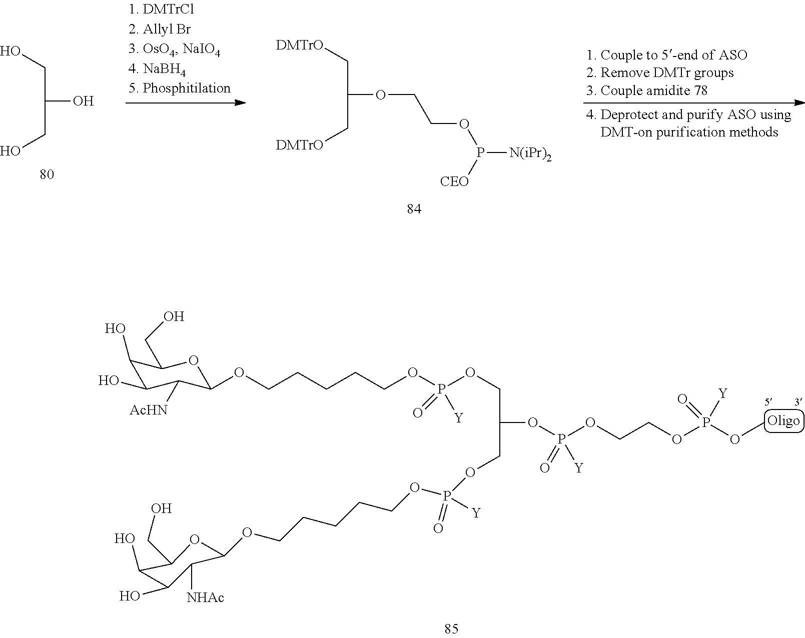

Oligonucleotide 85 comprises a GalNAc₂-32 conjugate group, wherein Y is selected from O, S, a substituted or unsubstituted C₁-C₁₀ alkyl, amino, substituted amino, azido, alkenyl or alkynyl. The GalNAc₂ cluster portion (GalNAc₂-32ₐ) of the conjugate group GalNAc₂-32 can be combined with any cleavable moiety to provide a variety of conjugate groups. In certain embodiments, the Y-containing group directly adjacent to the 5′-end of the oligonucleotide is part of the cleavable moiety. In certain embodiments, the Y-containing group directly adjacent to the 5′-end of the oligonucleotide is part of a stable moiety, and the cleavable moiety is present on the oligonucleotide. The structure of GalNAc₂-32ₐ is shown below:

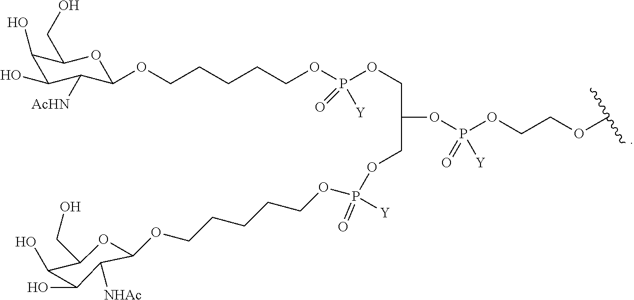

### Example 16: Synthesis of Oligonucleotides Comprising a GalNAc Modified at the C6 Position

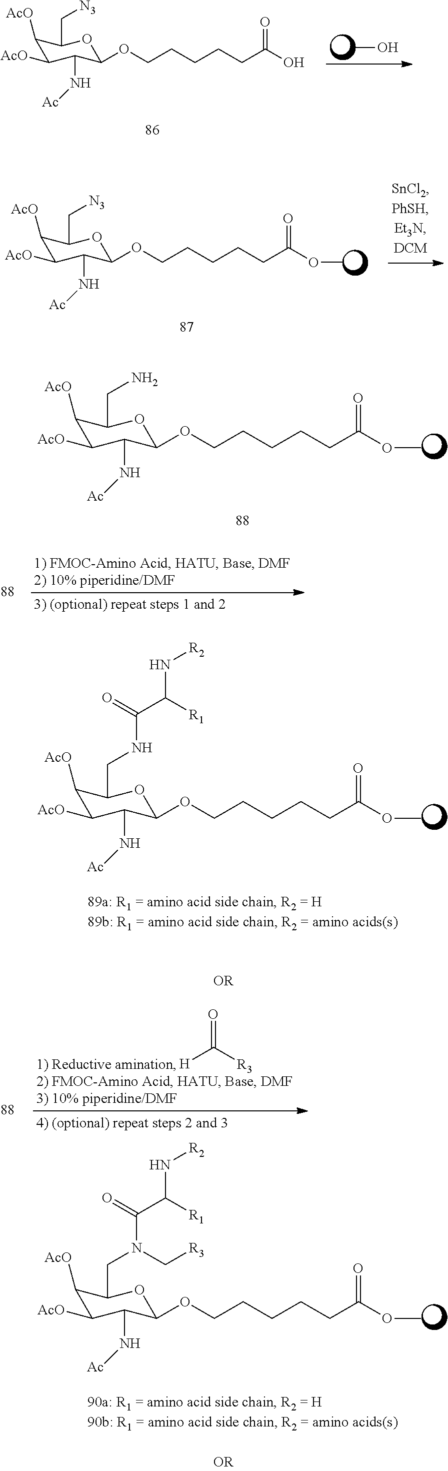

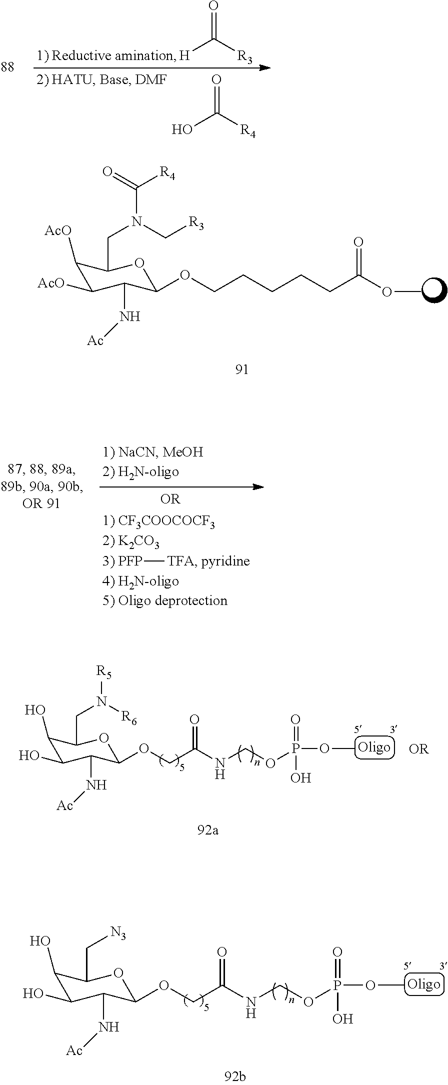

Compounds 87, 88, 89a, 89b, 90a, 90b, and 91 can be conjugated to an oligonucleotide of any sequence, resulting in a combinatorial library of oligonucleotides comprising a GalNAc conjugate, wherein the GalNAc is modified at the C6 position. The C6 position of the final product, compound 92a, comprises a primary amine (R₅ and R₆═H), an amino acid (R₅═H, R₆=amino acid) a peptide (R₅═H, R₆=peptide), or an alkylated amine (R₅═—CH₂—R₃, R₆=amino acid, peptide, or —C(O)—R₄, wherein R₃ and R₄ are substituent groups including but not limited to alkyl, alkenyl, and alkynyl groups); or the final product (compound 92b) comprises an azide. In 92a and 92b, n=1, 2, 3, 4, 5, or 6.

### Example 17: Synthesis of Oligonucleotides Comprising a GalNAc Modified at the C2 Position

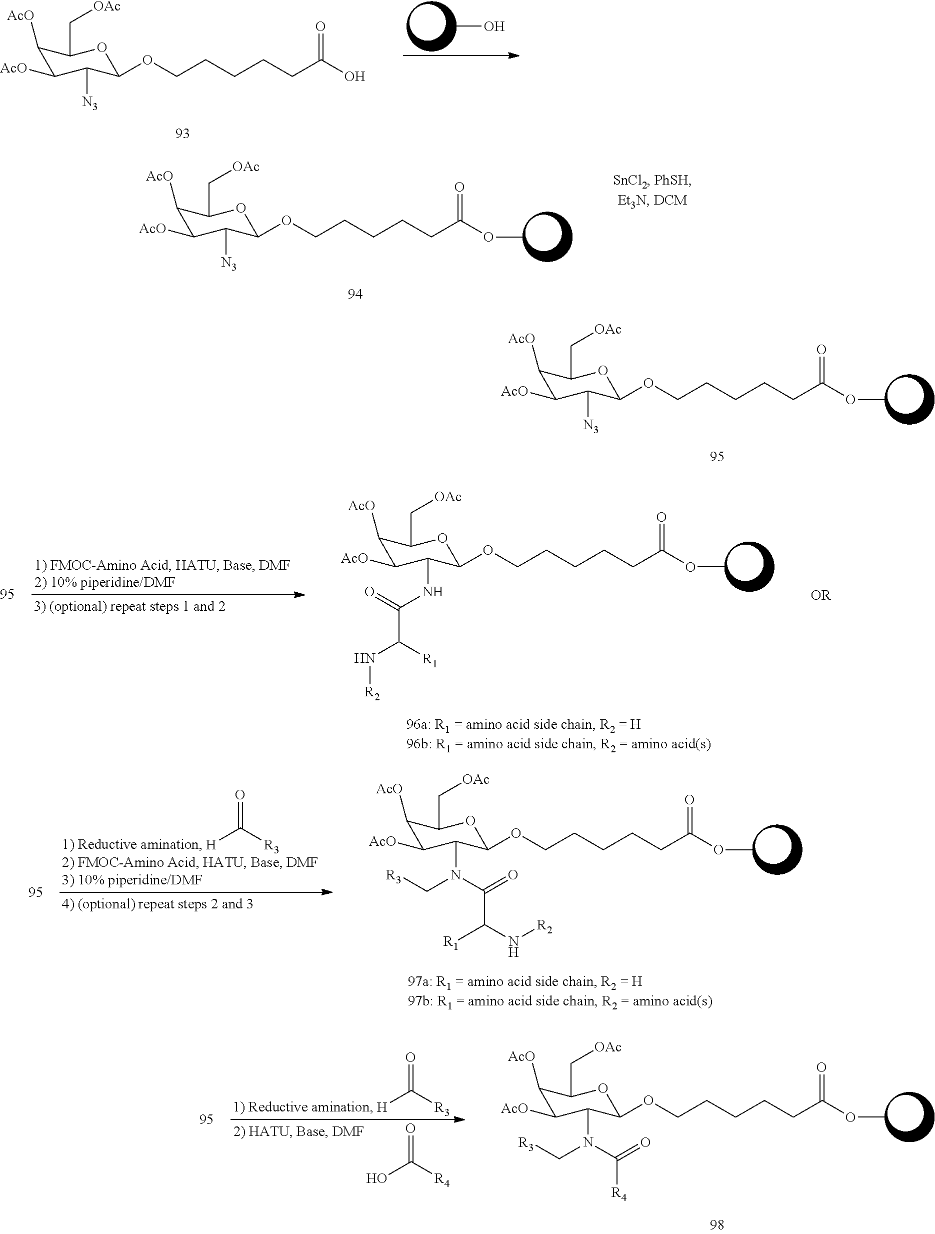

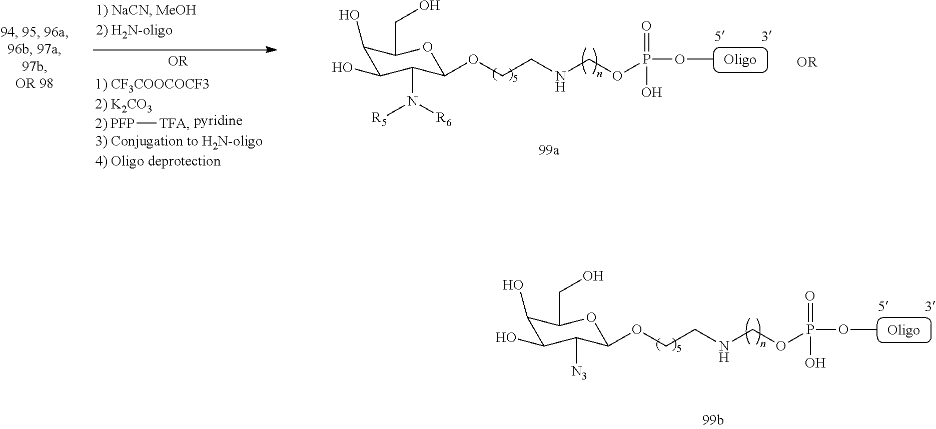

Compounds 94, 95, 96a, 96b, 97a, 97b, and 98 can be conjugated to an oligonucleotide of any sequence, resulting in a combinatorial library of oligonucleotides comprising a GalNAc conjugate, wherein the GalNAc is modified at the C2 position. The C2 position of the final product, compound 99a, comprises a primary amine (R₅ and R₆═H), an amino acid (R₅═H, R₆=amino acid) a peptide (R₅═H, R₆=peptide), or an alkylated amine (R₅═—CH₂—R₃, R₆=amino acid, peptide, or —C(O)—R₄, wherein R₃ and R₄ are substituent groups including but not limited to alkyl, alkenyl, and alkynyl groups); or the final product (compound 99b) comprises an azide. In 99a and 99b, n=1, 2, 3, 4, 5, or 6.

### Example 18: Synthesis of Oligonucleotides Comprising a GalNAc Modified at the C2 and C6 Positions

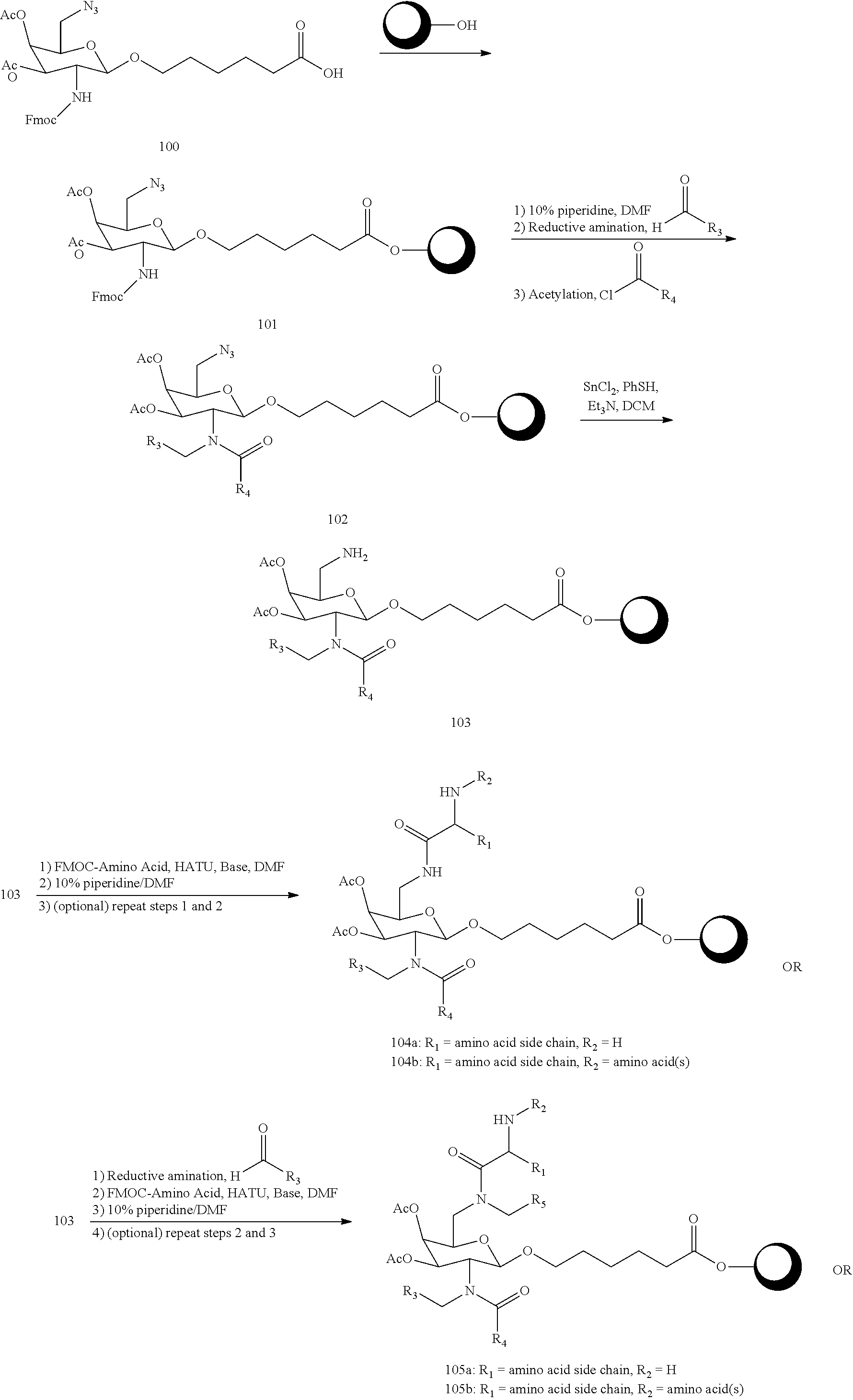

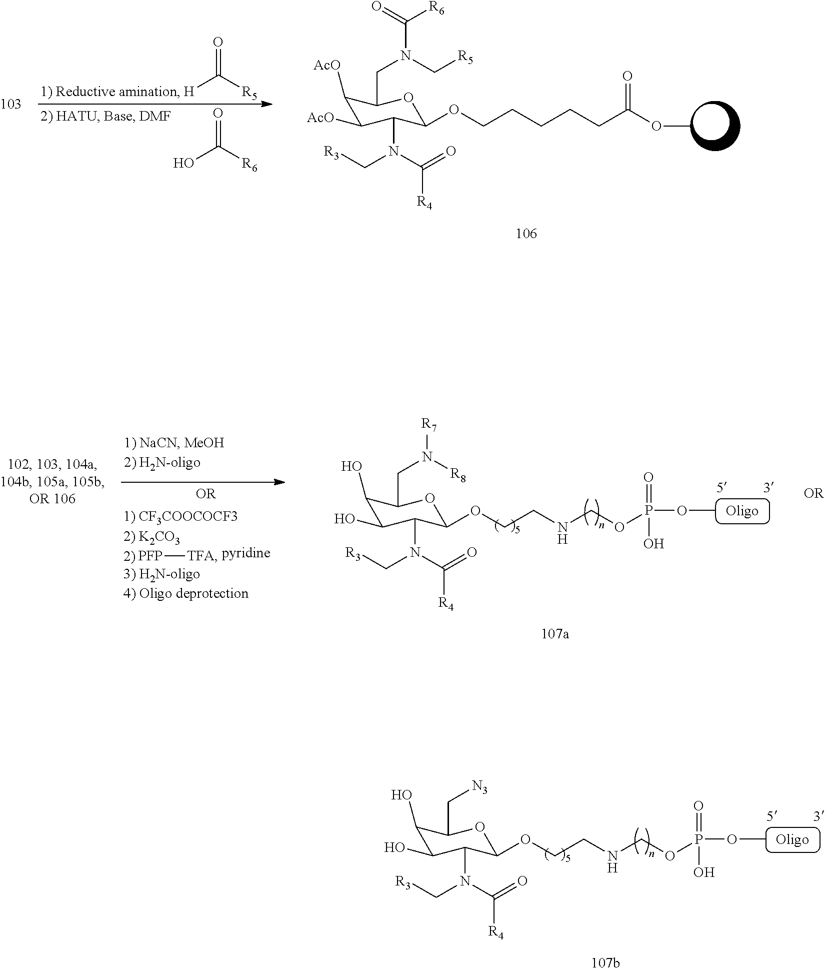

Compounds 102, 103, 104a, 104b, 105a, 105b, and 106 can be conjugated to an oligonucleotide of any sequence, resulting in a combinatorial library of oligonucleotides comprising a GalNAc conjugate, wherein the GalNAc is modified at the C2 and C6 positions. The C6 position of the final product, compound 107a, comprises a primary amine (R₇ and R₈═H), an amino acid (R₇═H, R₅=amino acid) a peptide (R₇═H, R₈=peptide), or an alkylated amine (R₇═—CH₂—R₅, R₈=amino acid, peptide, or —C(O)—R₆, wherein R₅ and R₆ are substituent groups including but not limited to alkyl, alkenyl, and alkynyl groups); or the C₆ position of the final product (compound 107b) comprises an azide. The C2 position of the final products, compounds 107a and 107b, comprises a substituted amine, wherein R₃ and R₄ are substituent groups including but not limited to alkyl, alkenyl, and alkynyl groups. In 107a and 107b, n=1, 2, 3, 4, 5, or 6.

### Example 19: Synthesis of Oligonucleotides Comprising Three Beta GalNAc Moieties Modified at the Anomeric Positions

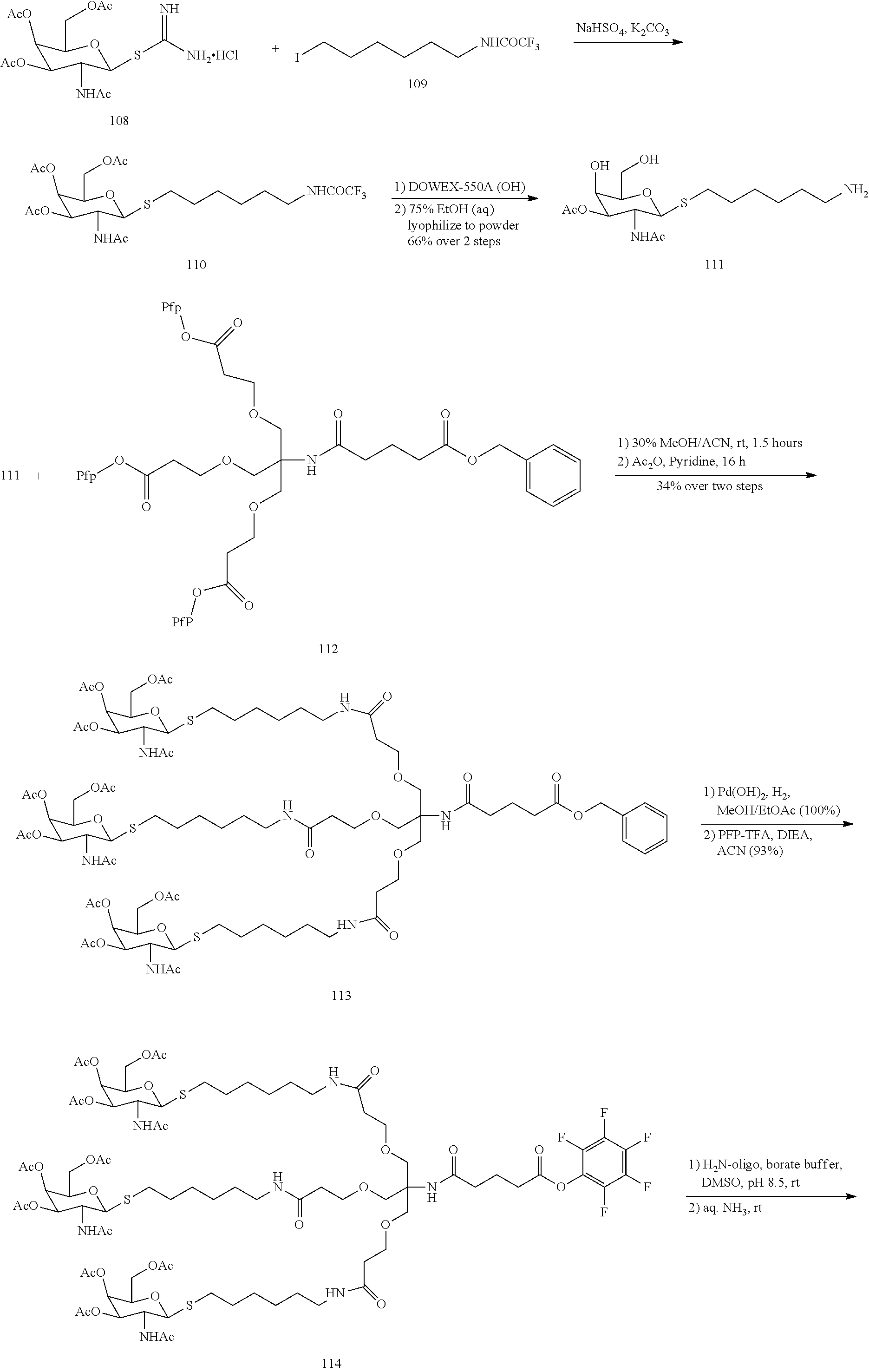

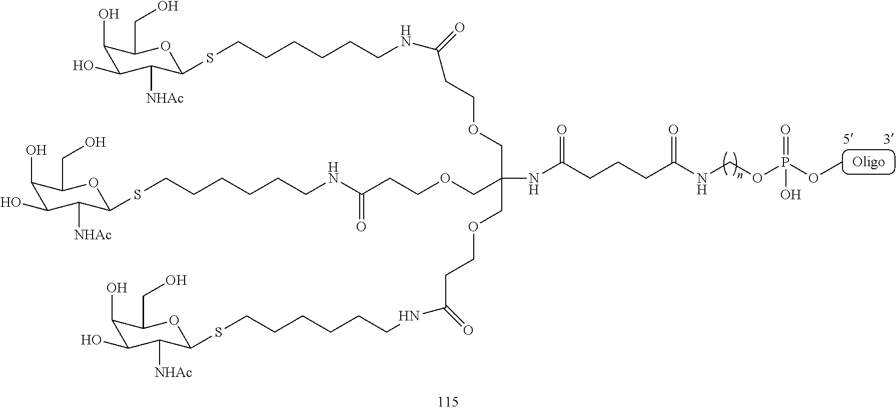

Compound 114 can be conjugated to an oligonucleotide of any sequence, resulting in a variety of oligonucleotides represented by compound 115, wherein n=1, 2, 3, 4, 5, or 6. For example, compound 114 was conjugated to a 5′-hexylamino modified antisense oligonucleotide targeting mouse SRB-1 in order to prepare ISIS 709049, an example of compound 115, wherein n=6. The sequence of ISIS 709049 is 5′-AGCTTCAGTCATGACTTCCTT-3′ (SEQ ID NO: 141), wherein the cytosines are 5-methylcytosines, and the adenosine at the 5′-end is a 2′-deoxyadenosine that is the cleavable moiety linking the GalNAc conjugate to the oligonucleotide. The internucleoside linkages are phosphorothioate except for the linkage between the deoxyadenosine and guanosine, which is a phosphodiester linkage. The twenty phosphorothioate linked nucleotides of ISIS 709049 comprise a gapmer, wherein the wings comprise 2′-methoxyethyl (MOE) modifications and are each five nucleotides in length. The gap comprises 2′-deoxynucleotides and is 10 nucleotides in length.

### Example 20: Synthesis of Oligonucleotides Comprising Three Alpha GalNAc Moieties Modified at the Anomeric Positions

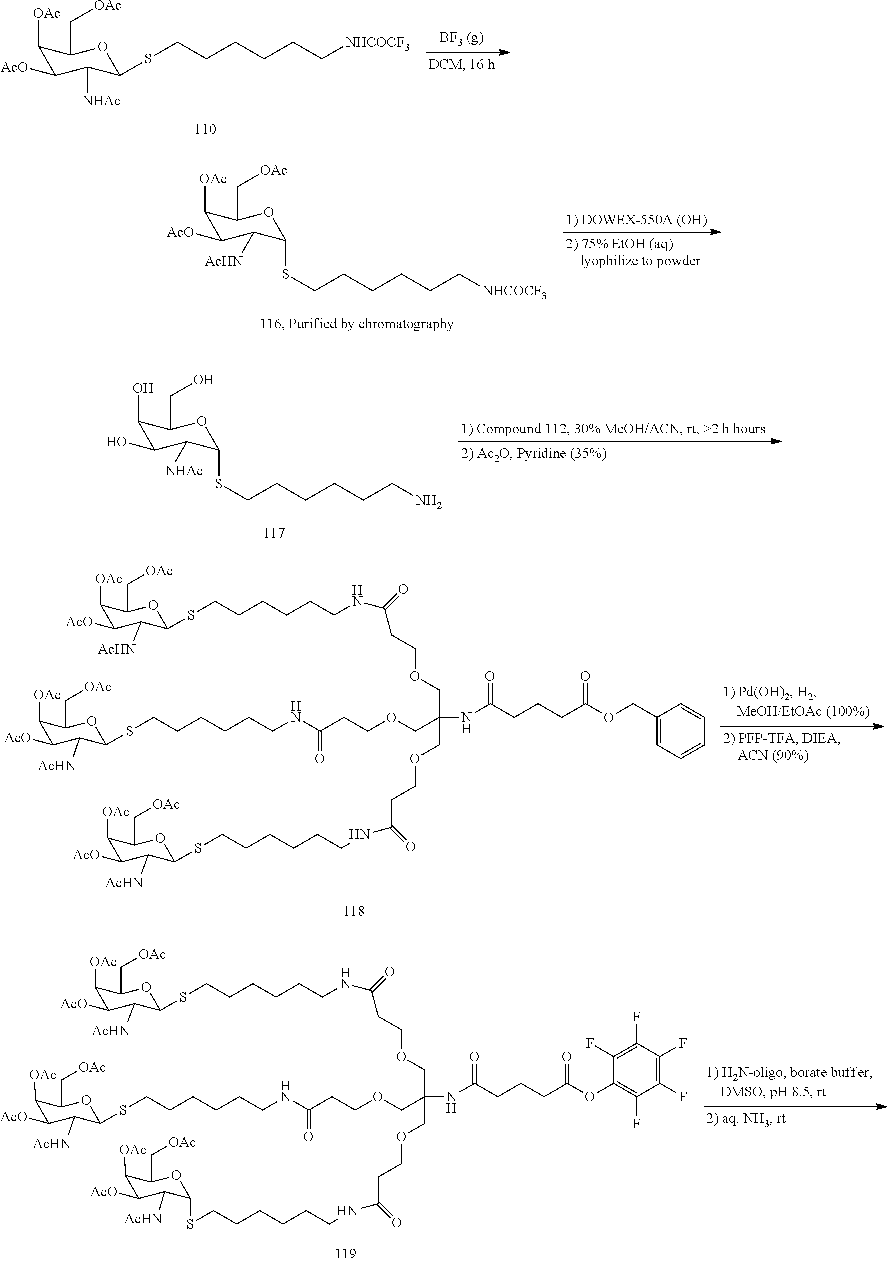

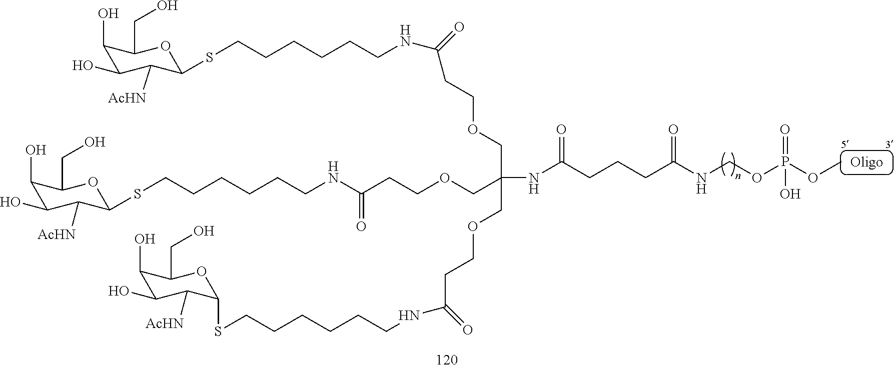

Compound 119 can be conjugated to an oligonucleotide of any sequence, resulting in a variety of oligonucleotides represented by compound 120, wherein n=1, 2, 3, 4, 5, or 6. For example, compound 119 was conjugated to a 5′-hexylamino modified antisense oligonucleotide targeting mouse SRB-1 in order to prepare ISIS 720333, an example of compound 120, wherein n=6. The sequence of ISIS 720333 is 5′-AGCTTCAGTCATGACTTCCTT-3′ (SEQ ID NO: 141), wherein the cytosines are 5-methylcytosines, and the adenosine at the 5′-end is a 2′-deoxyadenosine that is the cleavable moiety linking the GalNAc conjugate to the oligonucleotide. The internucleoside linkages are phosphorothioate except for the linkage between the deoxyadenosine and guanosine, which is a phosphodiester linkage. The twenty phosphorothioate linked nucleotides of ISIS 720333 comprise a gapmer, wherein the wings comprise 2′-methoxyethyl (MOE) modifications and are each five nucleotides in length. The gap comprises 2′-deoxynucleotides and is 10 nucleotides in length.

### Example 21: Synthesis of Oligonucleotides Comprising a Beta GalNAc Modified at the Anomeric Position

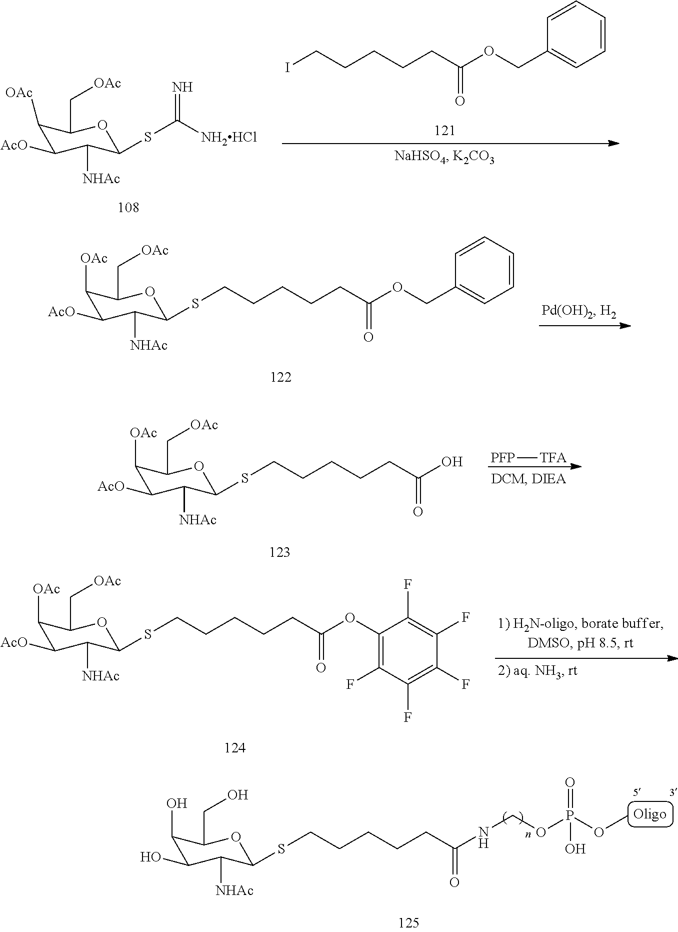

Compound 124 can be conjugated to an oligonucleotide of any sequence. In the final product, compound 125, n is 1, 2, 3, 4, 5, or 6. Alternatively, compound 129 below can be conjugated to the 5′-end of an oligonucleotide of any sequence using an automated oligonucleotide synthesizer, resulting in a variety of oligonucleotides represented by compound 130 below.

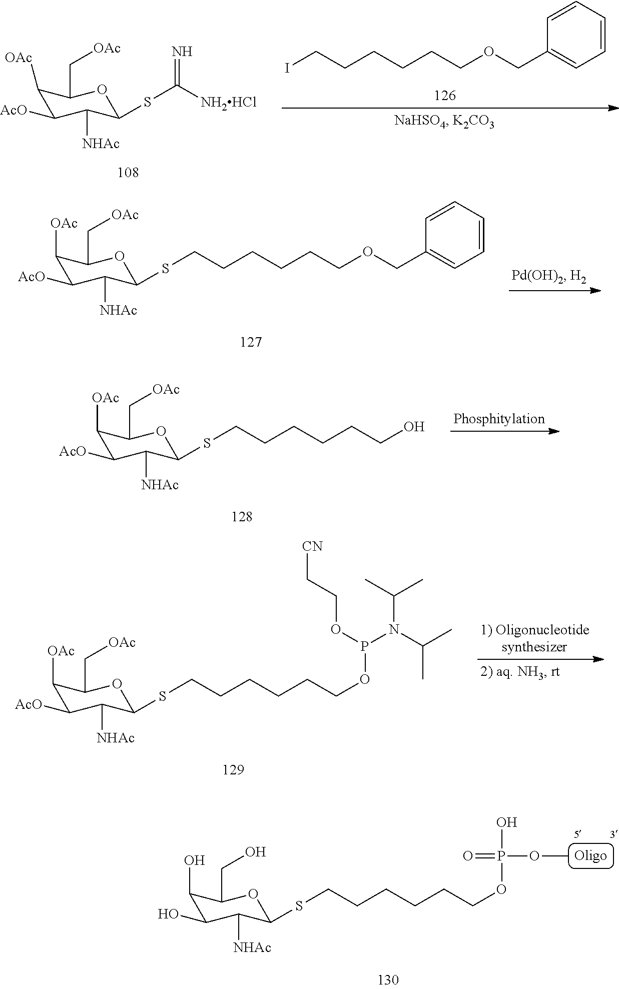

### Example 22: Synthesis of Oligonucleotides Comprising an Alpha GalNAc Modified at the Anomeric Position

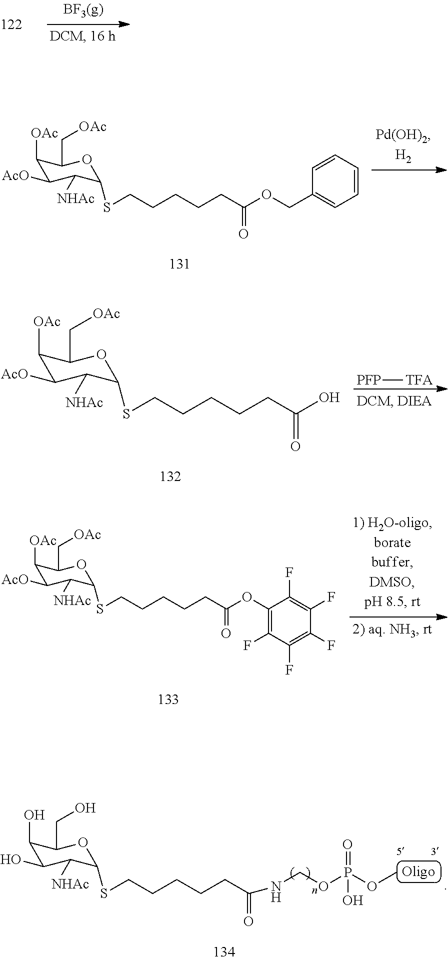

Compound 133 can be conjugated to an oligonucleotide of any sequence. In the final product, compound 134, n is 1, 2, 3, 4, 5, or 6. Alternatively, compound 137 below can be conjugated to the 5′-end of an oligonucleotide of any sequence using an automated oligonucleotide synthesizer, resulting in a variety of oligonucleotides represented by compound 138 below.

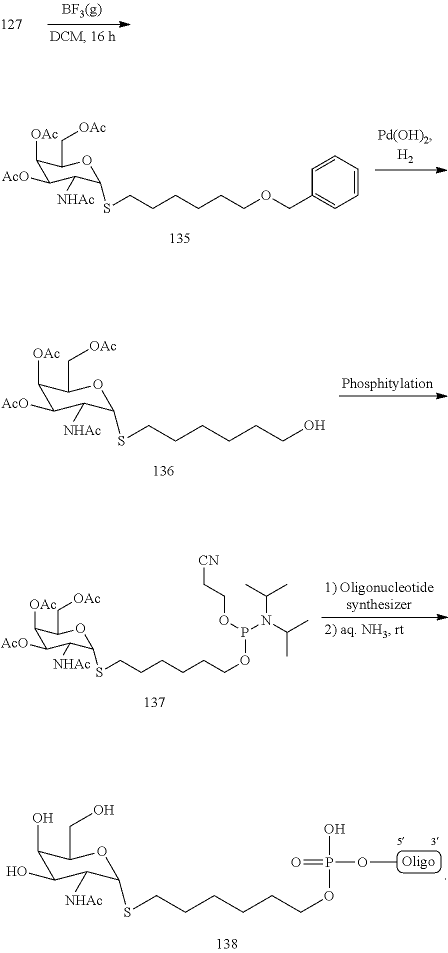 [SMILES: CC(=O)NC1C(SCCCCCCOCc2ccccc2)OC(COC(C)=O)C(OC(C)=O)C1OC(C)=O ; CC(=O)NC1C(SCCCCCCO)OC(COC(C)=O)C(OC(C)=O)C1OC(C)=O ; CC(=O)NC1C(SCCCCCCOP(OCCC#N)N(C(C)C)C(C)C)OC(COC(C)=O)C(OC(C)=O)C1OC(C)=O ; COP(=O)(O)OCCCCCCSC1OC(CO)C(O)C(O)C1NC(C)=O]

### Example 23: Synthesis of Oligonucleotides Comprising Three GalNAc Moieties Modified to Comprise a Triazole at the C6 Positions

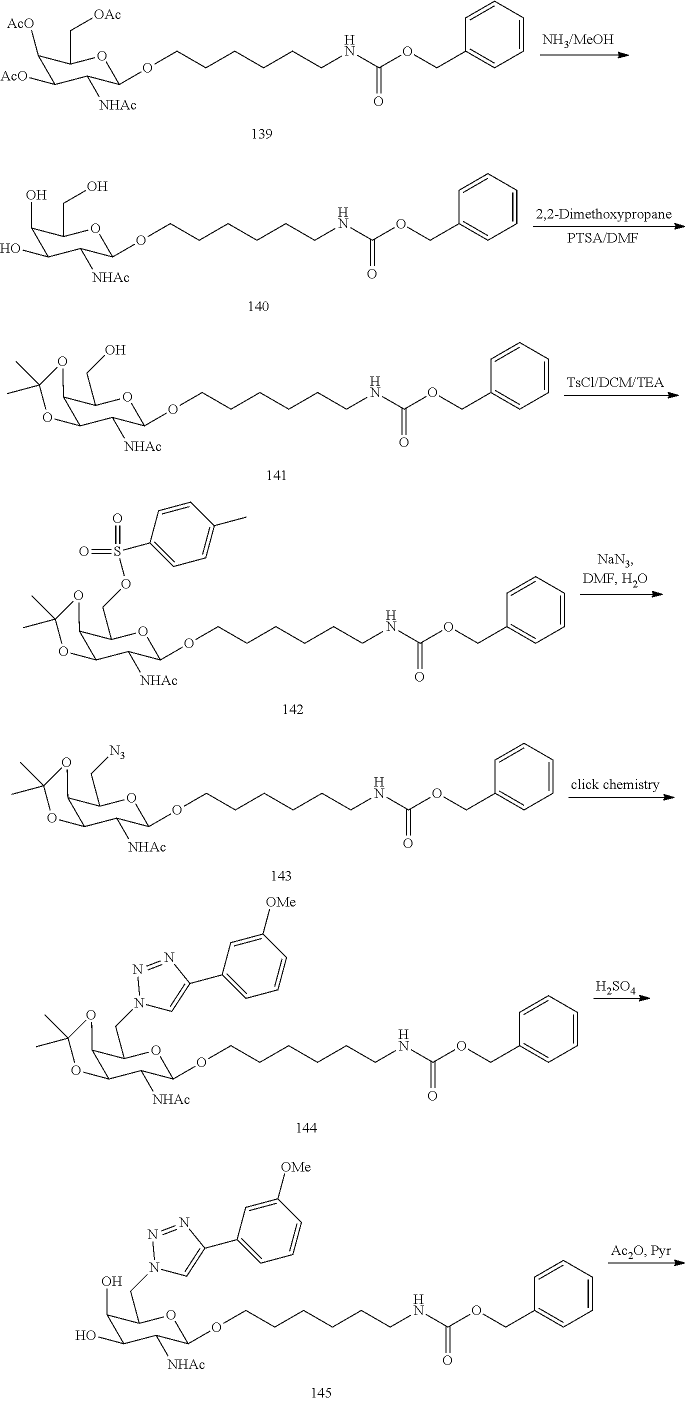

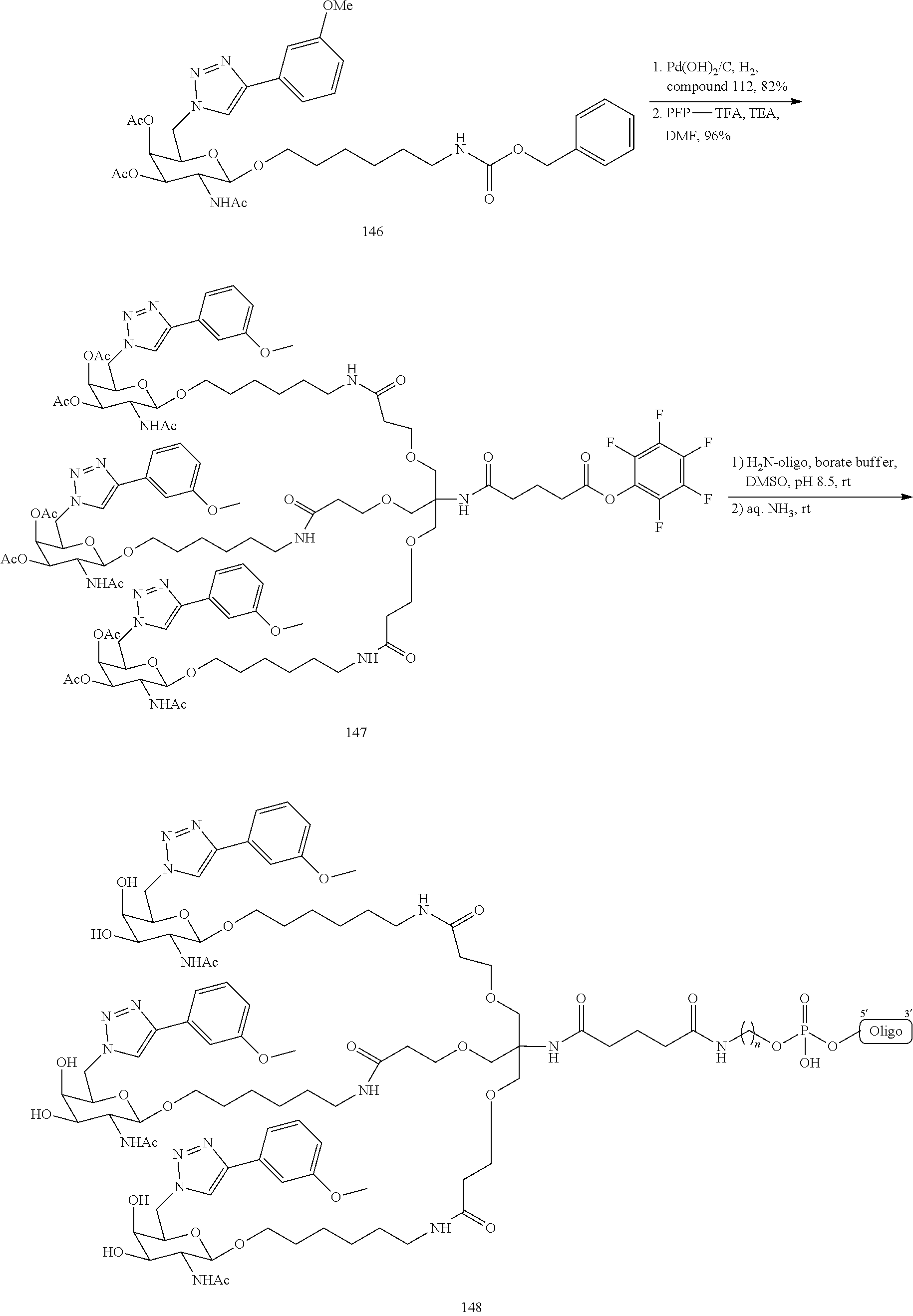

Compound 139 (5 g, 8.7 mmol) was dissolved in 7 N NH₃ in MeOH (30 mL) in a sealed 150 mL round-bottom flask and stirred at room temperature for 12 h. The clear solution became a thick white suspension. The reaction mixture was concentrated to dryness to yield a white solid, compound 140 (quantitative yield). Structure was confirmed by LCMS, ¹H NMR and ¹³C NMR analysis.

Compound 140 (3.9 g, 8.5 mmol), p-Toluenesulfonic acid monohydrate (0.15 g, 0.79 mmol) and 2,2-Dimethoxypropane (15 mL, 121.8 mmol) were suspended in DMF (20 mL) and stirred at room temperature for 12 h. 50% aqueous acetic acid (10 mL) was added and stirring continued for additional h. Solvent was removed under reduced pressure, and the residue was dissolved in 10% MeOH in DCM (200 mL) and washed with aqueous saturated NaHCO₃ solution and brine, dried (Na₂SO₄), filtered and concentrated. The residue was purified by silica gel column chromatography and eluted first with 50% ethyl acetate in DCM (5 CV), then with 100% ethyl acetate (10 CV) to yield compound 141 (1.83 g, 43.5%). Structure was confirmed by LCMS, ¹H NMR and ¹³C NMR analysis.

To a solution of compound 141 (4.1 g, 8.3 mmol) in dichloromethane (50 mL), triethylamine (3.5 mL, 18.3 mmol) was added and the reaction mixture was cooled in an ice bath. To this, a solution of p-toulenesulfonyl chloride (3.5 g, 18.3 mmol) in dichloromethane ion (30 mL) was added. The reaction mixture was allowed to come to room temperature and stirred for 72 h. The reaction was diluted with dichloromethane and washed with aqueous saturated NaHCO₃ solution and brine, dried (Na₂SO₄), filtered and concentrated to yield compound 142 (6.82 g). The structure was confirmed by LCMS, ¹H NMR and ¹³C NMR analysis.

To a solution of compound 142 (5.4 g, 8.3 mmol) in DMSO (40 mL) NaN₃ (6.8 g, 105 mmol) and water (6 mL) were added and the solution was heated at 100° C. for 25 h. The reaction mixture was diluted with ethyl acetate and with aqueous saturated NaHCO₃ solution and brine, dried (Na₂SO₄), filtered and concentrated. The residue obtained was purified silica gel column chromatography and eluted with 10-40% acetone in dichloromethane to yield compound 143 (3.35 g, 77.6%). Structure was confirmed by LCMS, ¹H NMR and ¹³C NMR analysis.

To a solution of 3-ethynylanisole (0.88 mL, 6.9 mmol) and compound 143 (3 g, 5.8 mmol) in MeOH (20 mL), TBTA (tris[(1-benzyl-1H-1,2,3-triazol-4-yl)methyl]amine (0.15 g, 0.29 mmol), CuSO4·5H2O (0.014 g, 0.058 mmol) in water (2 mL, reaction became blue solution), and (+)-Sodium L-ascorbate (0.11 g, 0.58 mmol) in water (1 ml, reaction color changed to yellow) were added. The reaction was vigorously stirred for 12 h at room temperature, and concentrated to dryness. The residue was the dissolved in dichloromethane (100 mL) and washed with water (50 mL×3). The organic phase was separated and the aqueous phase was further extracted with (2×10 mL). The combined organic fractions were concentrated and the residue was purified by Biotage silica gel (100 g) chromatography that eluted with 15% (6 CV), 20% (6 CV), 25% (6 CV) and 30% (6 CV) acetone in dichloromethane to yield compound 144 (3.6 g, 95.7%). Structure was confirmed by LCMS, ¹H NMR and ¹³C NMR analysis.

Compound 144 (3.35 g, 5.14 mmol) was dissolved in acetonitrile (57 mL) and aqueous H₂SO₄(1.84%, 43 mL) was added. The mixture was stirred at room temperature for 96 h. The reaction mixture was extracted with ethyl acetate and washed with aqueous saturated NaHCO₃ and brine. The organic phase was concentrated to dryness and the crude product was purified through silica gel column and eluted with 2-10% MeOH in dichloromethane to yield compound 145 (2.92 g, 93%). Structure was confirmed by LCMS, ¹H NMR and ¹³C NMR analysis.

Compound 145 (1.78 g, 2.9 mmol) was dissolved in anhydrous pyridine (45 mL) and to this acetic anhydride (2.75 mL, 29 mmol) was added. The reaction mixture was stirred at room temperature for 12 h and then at 50° C. for 3 h. The reaction mixture was extracted with dichloromethane (150 mL) and the dichloromethane phase was washed with aqueous saturated sodium solution (100 mL), brine (100 mL), 2N HCl (100 mL) and brine (100 mL). The organic phase was dried over Na₂SO₄, filtered and concentrated to dryness. The residue was purified by silica gel column chromatography and eluted with 1-5% MeOH in dichloromethane to yield compound 146 (1.96 g, 96.8%). Structure was confirmed by LCMS, ¹H NMR and ¹³C NMR analysis.

Compound 146 (1.62 g, 2.31 mmol) and compound 112 (0.8 g, 0.77 mmol) were dissolved in THF (16 mL). To this mixture, Pd(OH)₂ (0.28 g) was added. The reaction mixture was stirred at room temperature for 3 h. The suspension was filtered through a pad of Celite and washed with THF. The organic phase were combined and concentrated to dryness under reduced pressure. The residue was purified by silica gel chromatography and eluted with 5-20% MeOH in dichloromethane to yield tri-antinary cluster acid (1.12 g, 70%). The cluster acid (1 g, 0.48 mmol) and TEA (0.2 mL, 1.44 mmol) were dissolved in dichloromethane (10 mL) and PFP-TFA (0.16 mL, 0.96 mmol) was added. After two h, the reaction mixture was diluted with dichloromethane and washed with 1N NaHSO₄ (30 mL×2), brine (30 mL); aqueous saturated sodium bicarbonate (30 mL×2), and brine (30 mL). The resulting solution was dried over Na₂SO₄, filtered and concentrated to dryness under reduced pressure to yield compound 147 (1.05 g, 97%). Structure was confirmed by LCMS, ¹H NMR and ¹³C NMR analysis.

Compound 147 can be conjugated to an oligonucleotide of any sequence, resulting in a variety of oligonucleotides represented by compound 148, wherein n=1, 2, 3, 4, 5, or 6.

### Example 24: Synthesis of Oligonucleotides Comprising a GalNAc Modified to Comprise a Triazole at the C6 Position

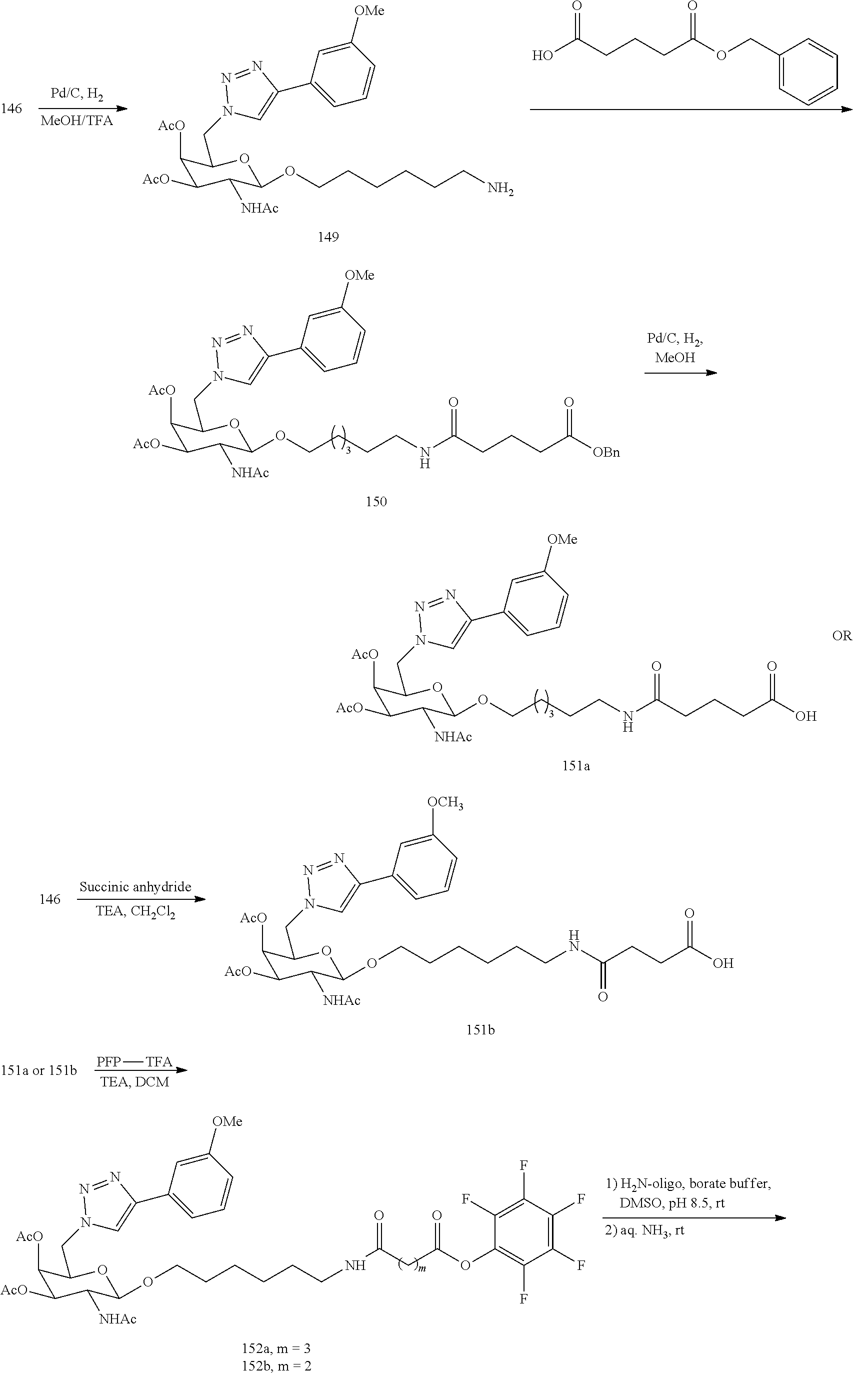

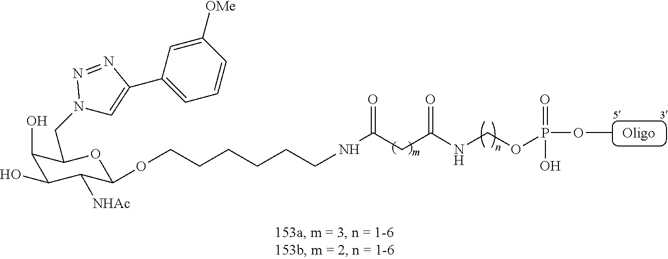

Compounds 152a and 152b can be conjugated to an oligonucleotide of any sequence, resulting in a PGP variety of oligonucleotides represented by compounds 153a and 153b, wherein n=1, 2, 3, 4, 5, or 6. Alternatively, compound 154 below can be conjugated to the 5′-end of an oligonucleotide of any sequence using an automated oligonucleotide synthesizer, resulting in a variety of oligonucleotides represented by compound 155 below.

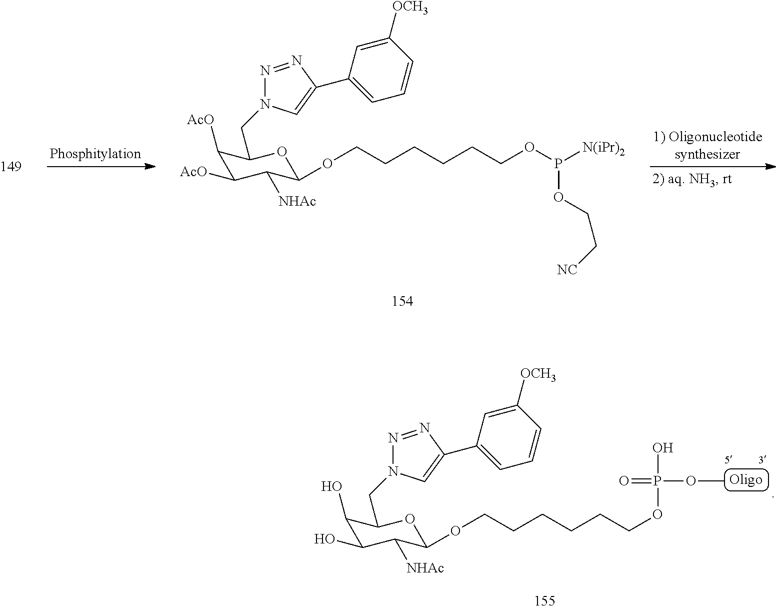

### Example 25: Synthesis of Oligonucleotides Comprising a GalNAc Modified to Comprise an Amide at the C6 Position

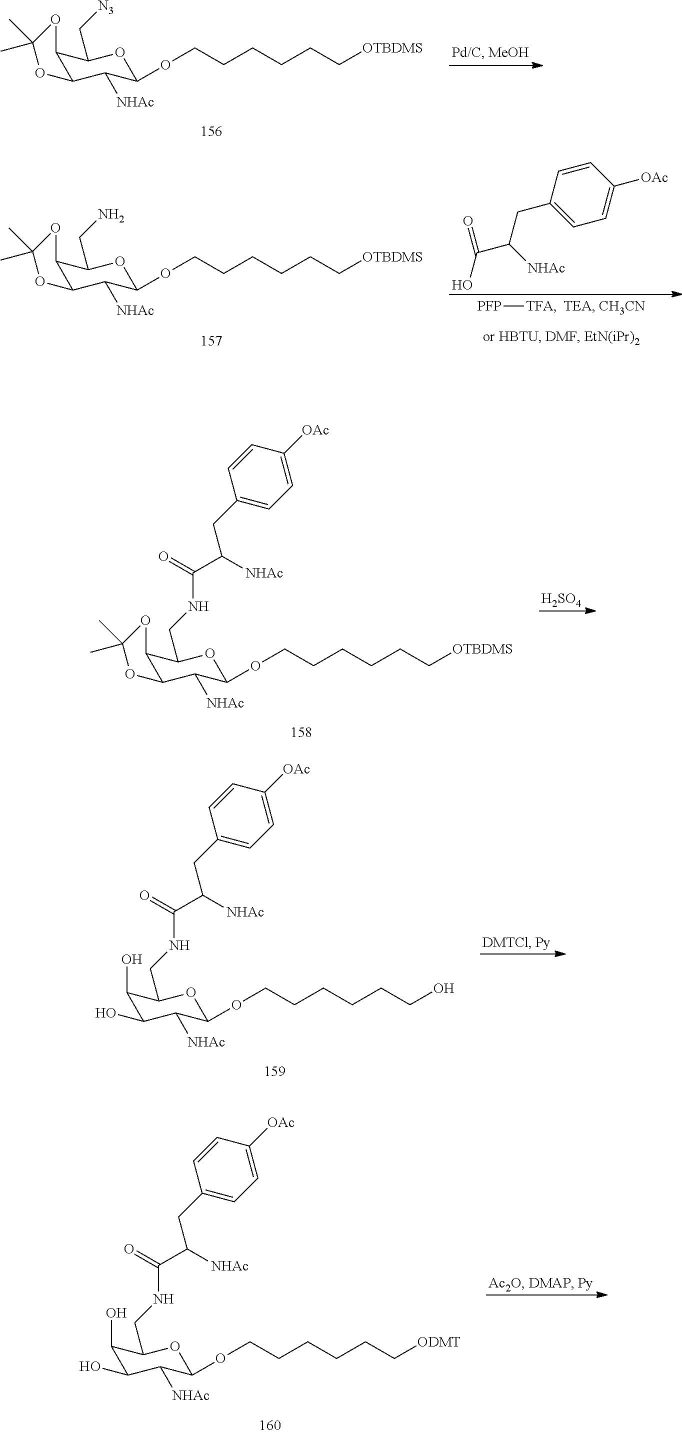

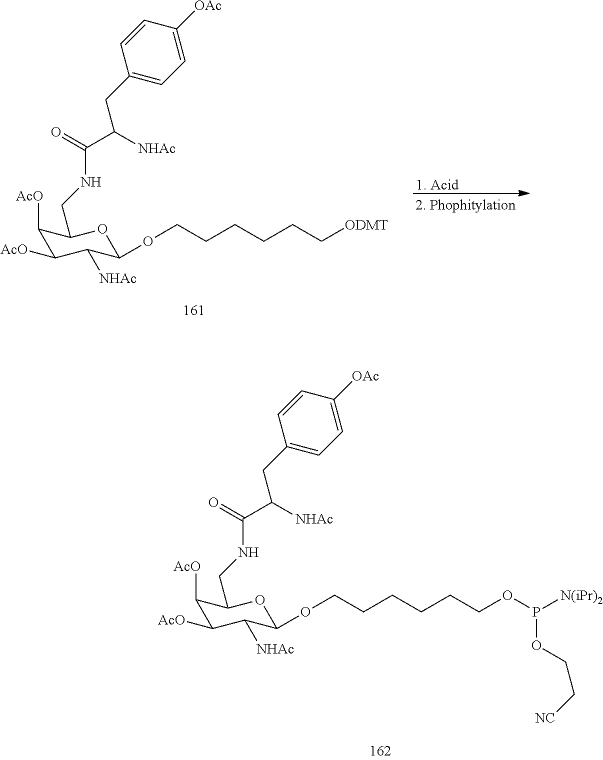

Compound 162 can be conjugated to the 5′-end of an oligonucleotide of any sequence using an automated oligonucleotide synthesizer, resulting in a variety of oligonucleotides.

### Example 26: Synthesis of Oligonucleotides Comprising a GalNAc Modified to Comprise an Indole Moiety at the C6 Position

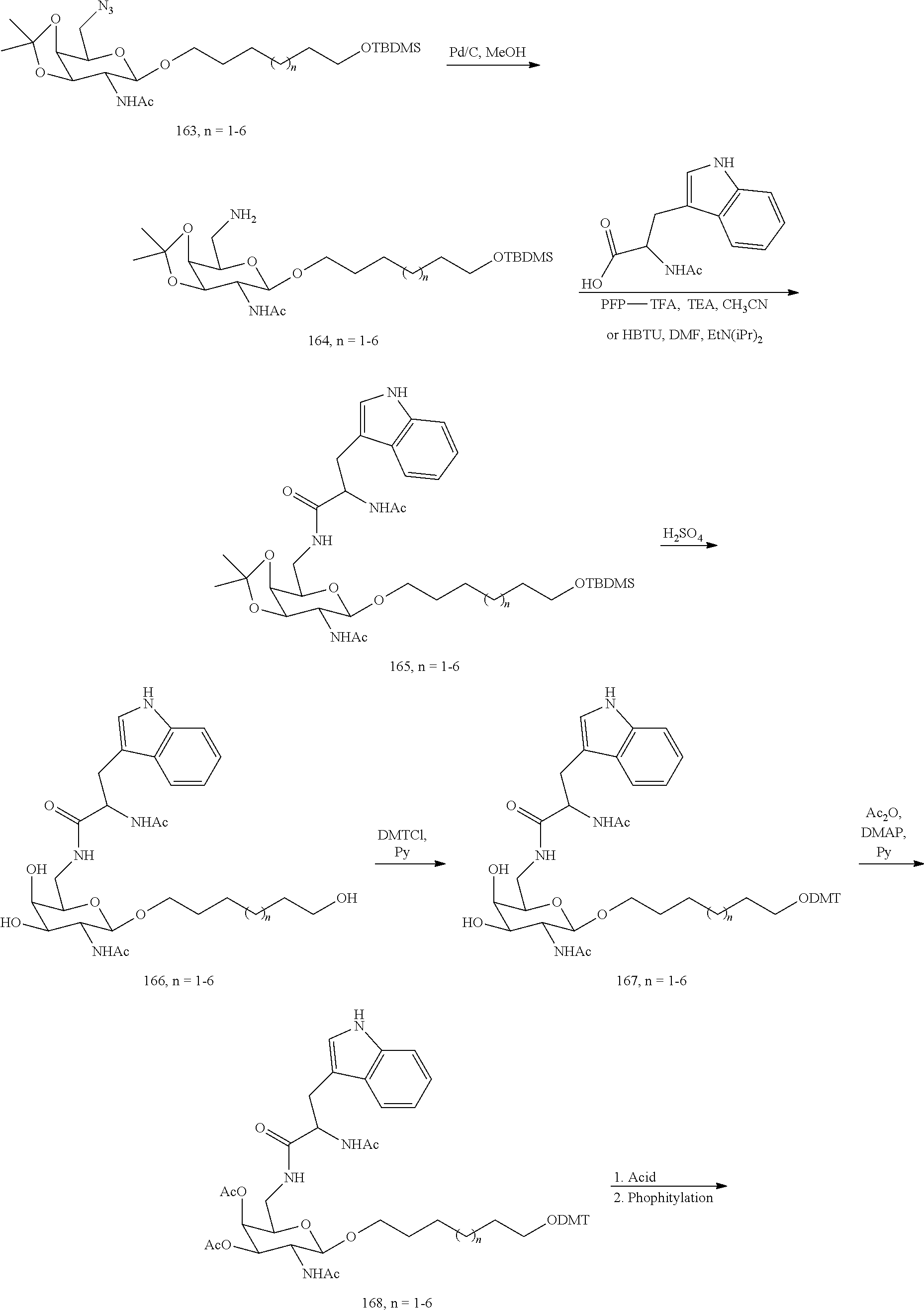

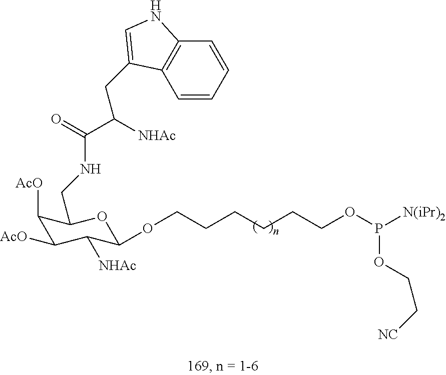

Compound 169 can be conjugated to the 5′-end of an oligonucleotide of any sequence using an automated oligonucleotide synthesizer, resulting in a variety of oligonucleotides.

### Example 27: Synthesis of Oligonucleotides Comprising a GalNAc Modified to Comprise an Azide at the C6 Position

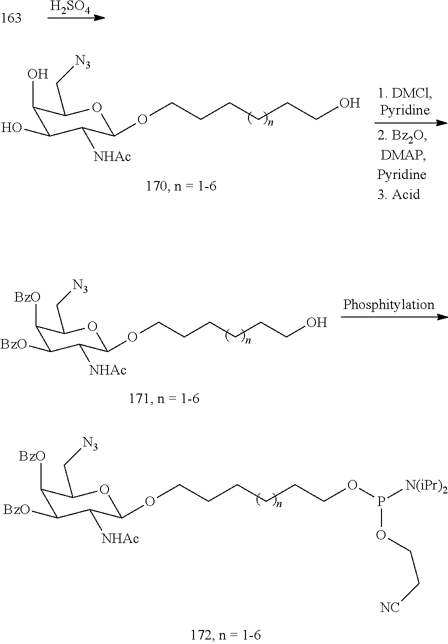

Compound 172 can be conjugated to the 5′-end of an oligonucleotide of any sequence using an automated oligonucleotide synthesizer, resulting in a variety of oligonucleotides.

### Example 28: Synthesis of Oligonucleotides Comprising a GalNAc Modified to Comprise a Triazole at the C6 Position

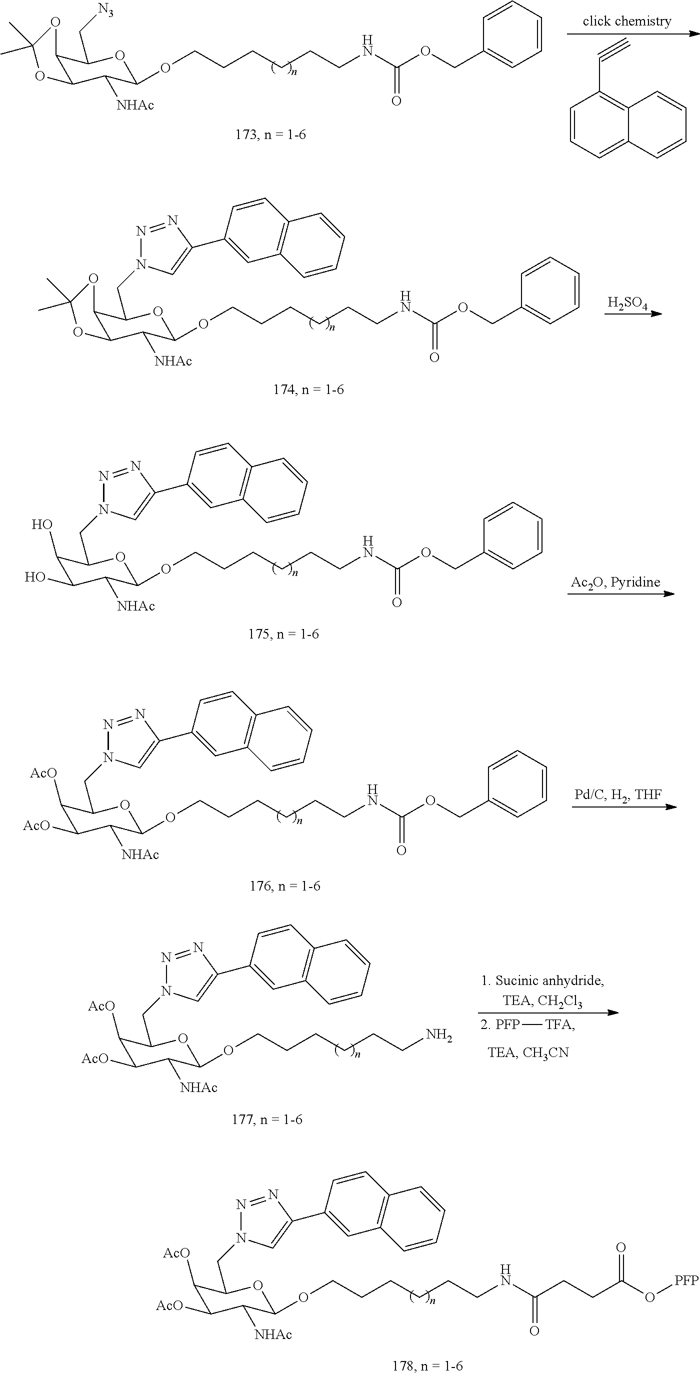

Compound 178 can be conjugated to an oligonucleotide of any sequence, resulting in a variety of oligonucleotides. Alternatively, compound 179 below can be conjugated to the 5′-end of an oligonucleotide of any sequence using an automated oligonucleotide synthesizer, resulting in a variety of oligonucleotides.

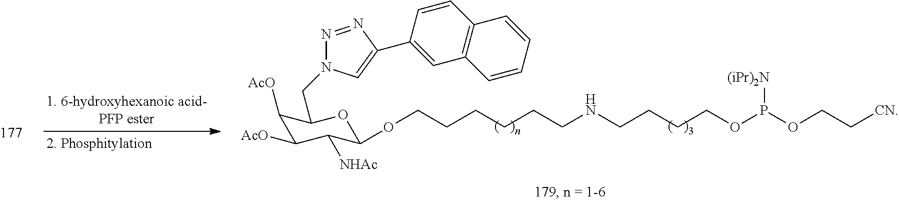

### Example 29: Synthesis of Oligonucleotides Comprising a GalNAc Modified to Comprise an Amide at the C6 Position

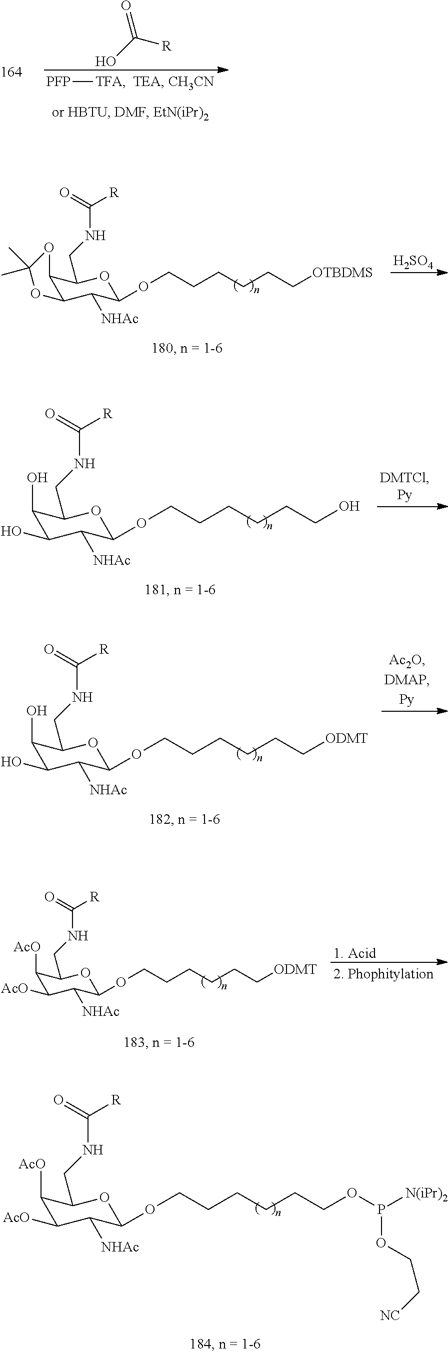

Using the method shown in the scheme above, a variety of GalNAc moieties modified to comprise an amide at the C6 position can be prepared, represented by compound 184. Phosphoramidite 184 can be conjugated to the 5′-end of an oligonucleotide of any sequence using an automated oligonucleotide synthesizer, resulting in a variety of oligonucleotides. Alternatively, the pentafluorophenyl ester of compound 183 can be synthesized and conjugated to an oligonucleotide of any sequence, resulting in a variety of oligonucleotides.

### Example 30: Synthesis of Oligonucleotides Comprising Three GalNAc Moieties Modified to Comprise an Amide at the C6 Position

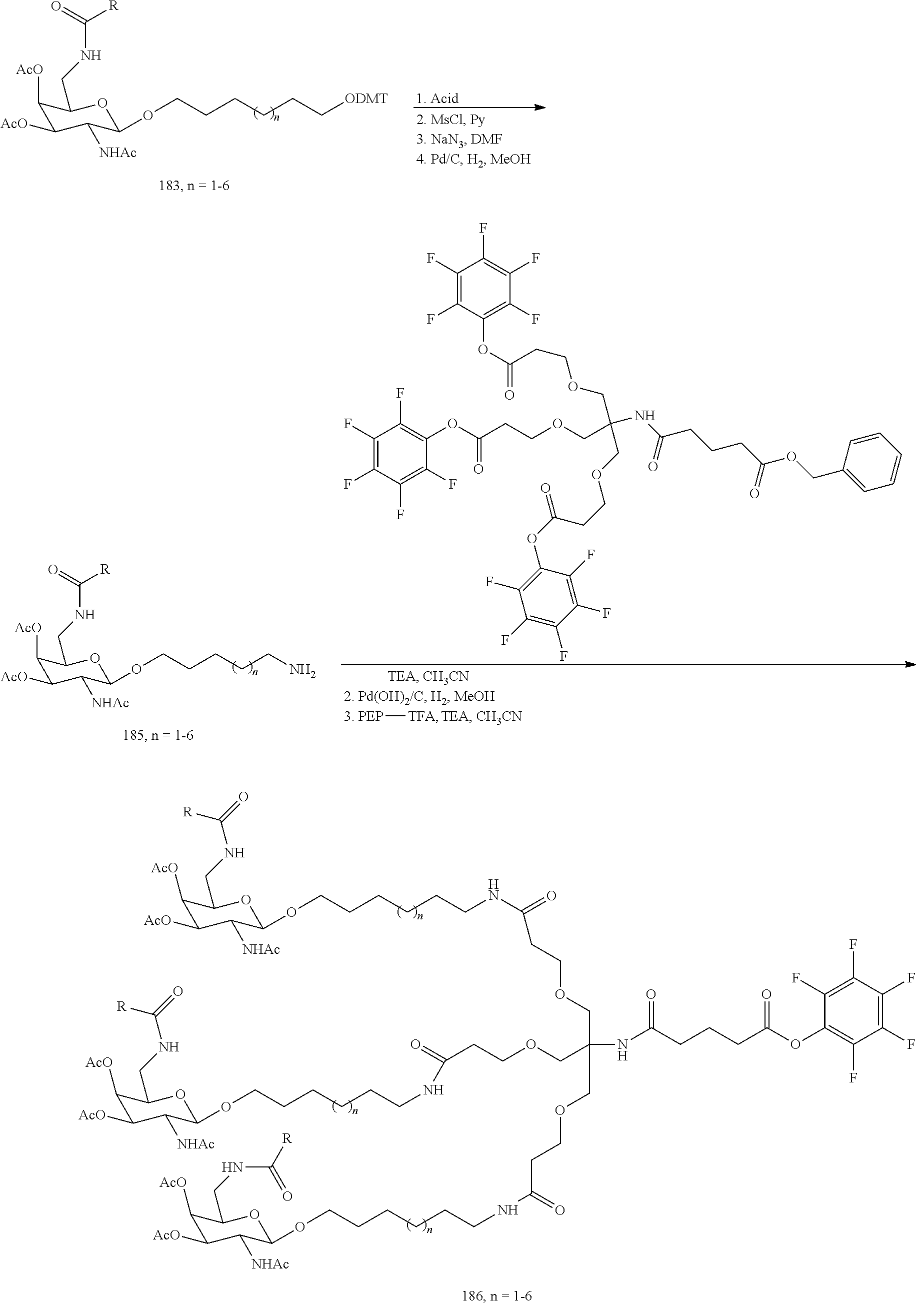

Using the method shown in the scheme above, a variety of trivalent GalNAc moieties modified to comprise an amide at the C6 position can be prepared, represented by compound 186. Compound 186 can be conjugated to an oligonucleotide of any sequence, resulting in a variety of oligonucleotides, wherein n=1, 2, 3, 4, 5, or 6.

### Example 31: Synthesis of Oligonucleotides Comprising a GalNAc Modified to Comprise a Triazole at the C2 Position

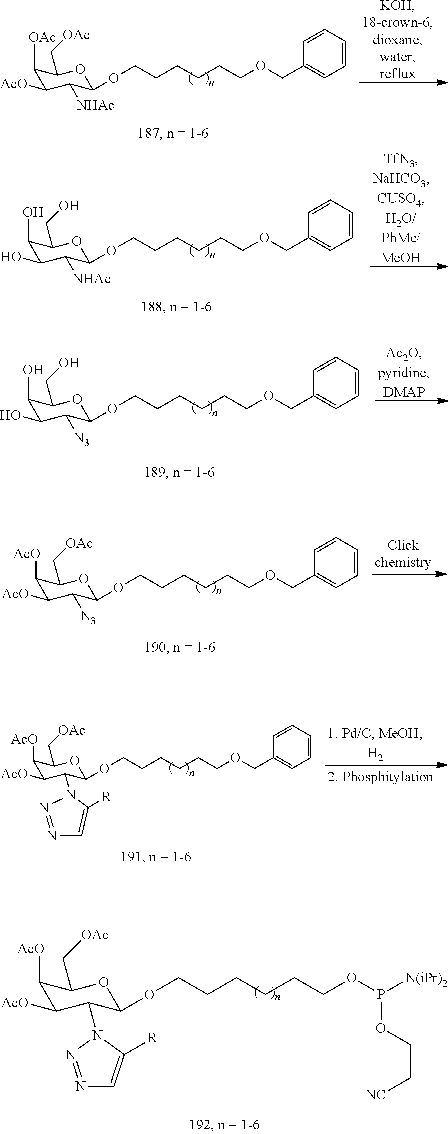

Using the method shown in the scheme above, a variety of GalNAc moieties modified to comprise a triazole at the C2 position can be prepared, represented by compound 192. Phosphoramidite 192 can be conjugated to the 5′-end of an oligonucleotide of any sequence using an automated oligonucleotide synthesizer, resulting in a variety of oligonucleotides. Alternatively, the pentafluorophenyl ester of deprotected compound 191 can be synthesized and conjugated to an oligonucleotide of any sequence, resulting in a variety of oligonucleotides.

### Example 32: Dose-Dependent Antisense Inhibition of Human ApoC III in huApoC III Transgenic Mice

ISIS 304801 and ISIS 647535, each targeting human ApoC III and described above, were separately tested and evaluated in a dose-dependent study for their ability to inhibit human ApoC III in human ApoC III transgenic mice.

### Treatment

Human ApoCIII transgenic mice were maintained on a 12-hour light/dark cycle and fed ad libitum Teklad lab chow. Animals were acclimated for at least 7 days in the research facility before initiation of the experiment. ASOs were prepared in PBS and sterilized by filtering through a 0.2 micron filter. ASOs were dissolved in 0.9% PBS for injection.

Human ApoC III transgenic mice were injected intraperitoneally once a week for two weeks with ISIS 304801 or 647535 at 0.08, 0.25. 0.75, 2.25 or 6.75 μmol/kg or with PBS as a control. Each treatment group consisted of 4 animals. Forty-eight hours after the administration of the last dose, blood was drawn from each mouse and the mice were sacrificed and tissues were collected.

ApoC III mRNA Analysis

ApoC III mRNA levels in the mice's livers were determined using real-time PCR and RIBOGREEN® RNA quantification reagent (Molecular Probes, Inc. Eugene, OR) according to standard protocols. ApoC III mRNA levels were determined relative to total RNA (using Ribogreen), prior to normalization to PBS-treated control. The results below are presented as the average percent of ApoC III mRNA levels for each treatment group, normalized to PBS-treated control and are denoted as “% PBS”. The half maximal effective dosage (ED₅₀) of each ASO is also presented in Table 26, below.

As illustrated, both antisense compounds reduced ApoC III RNA relative to the PBS control. Further, the antisense compound conjugated to GalNAc₃-1 (ISIS 647535) was substantially more potent than the antisense compound lacking the GalNAc₃-1 conjugate (ISIS 304801).

TABLE 26
Effect of ASO treatment on ApoC III mRNA
levels in human ApoC III transgenic mice

| | | | | | Internu- | |
|---|---|---|---|---|---|---|
| | | | ED₅₀ | | cleoside | |
| | Dose | % | (μmol/ | 3′ | linkage/ | SEQ |
| ASO | (μmol/kg) | PBS | kg) | Conjugate | Length | ID No. |
| PBS | 0 | 100 | — | — | — | |
| ISIS | 0.08 | 95 | 0.77 | None | PS/20 | 32 |
| 304801 | 0.75 | 42 | | | | |
| | 2.25 | 32 | | | | |
| | 6.75 | 19 | | | | |
| ISIS | 0.08 | 50 | 0.074 | GalNAc₃-1 | PS/20 | 111 |
| 647535 | 0.75 | 15 | | | | |
| | 2.25 | 17 | | | | |
| | 6.75 | 8 | | | | |

### ApoC III Protein Analysis (Turbidometric Assay)

Plasma ApoC III protein analysis was determined using procedures reported by Graham et al, Circulation Research, published online before print Mar. 29, 2013.

Approximately 100 μl of plasma isolated from mice was analyzed without dilution using an Olympus Clinical Analyzer and a commercially available turbidometric ApoC III assay (Kamiya, Cat4 KAI-006, Kamiya Biomedical, Seattle, WA). The assay protocol was performed as described by the vendor.

As shown in the Table 27 below, both antisense compounds reduced ApoC III protein relative to the PBS control. Further, the antisense compound conjugated to GaNAc₃-1 (ISIS 647535) was substantially more potent than the antisense compound lacking the GalNAc₃-1 Conjugate (ISIS 304801).

TABLE 27
Effect of ASO treatment on ApoC III plasma protein
levels in human ApoC III transgenic mice

| | | | | | Internu- | |
|---|---|---|---|---|---|---|
| | | | ED₅₀ | | cleoside | |
| | Dose | % | (μmol/ | 3′ | Linkage/ | SEQ |
| ASO | (μmol/kg) | PBS | kg) | Conjugate | Length | ID No. |
| PBS | 0 | 100 | — | — | — | |
| ISIS | 0.08 | 86 | 0.73 | None | PS/20 | 32 |
| 304801 | 0.75 | 51 | | | | |
| | 2.25 | 23 | | | | |
| | 6.75 | 13 | | | | |
| ISIS | 0.08 | 72 | 0.19 | GalNAc₃-1 | PS/20 | 111 |
| 647535 | 0.75 | 14 | | | | |
| | 2.25 | 12 | | | | |
| | 6.75 | 11 | | | | |

Plasma triglycerides and cholesterol were extracted by the method of Bligh and Dyer (Bligh, E. G. and Dyer, W. Can. J. Biochem. Physiol. 37: 911-917, 1959)(Bligh, and Dyer, W, Can J Biochem Physiol, 37, 911-917, 1959)(Bligh, E and Dyer, W, Can J Biochem Physiol, 37, 911-917, 1959) and measured by using a Beckmann Coulter clinical analyzer and commercially available reagents.

The triglyceride levels were measured relative to PBS injected mice and are denoted as “PBS”. Results are presented in Table 28. As illustrated, both antisense compounds lowered triglyceride levels. Further, the antisense compound conjugated to GalNAc₃-1 (ISIS 647535) was substantially more potent than the antisense compound lacking the GalNAc₃-1 Conjugate (ISIS 304801).

TABLE 28
Effect of ASO treatment on triglyceride levels in transgenic mice

| | | | | | Internu- | |
|---|---|---|---|---|---|---|
| | | | ED₅₀ | | cleoside | |
| | Dose | % | (μmol/ | 3′ | Linkage/ | SEQ |
| ASO | (μmol/kg) | PBS | kg) | Conjugate | Length | ID No. |
| PBS | 0 | 100 | — | — | — | |
| ISIS | 0.08 | 87 | 0.63 | None | PS/20 | 32 |
| 304801 | 0.75 | 46 | | | | |
| | 2.25 | 21 | | | | |
| | 6.75 | 12 | | | | |
| ISIS | 0.08 | 65 | 0.13 | GalNAc₃-1 | PS/20 | 111 |
| 647535 | 0.75 | 9 | | | | |
| | 2.25 | 8 | | | | |
| | 6.75 | 9 | | | | |

Plasma samples were analyzed by HPLC to determine the amount of total cholesterol and of different fractions of cholesterol (HDL and LDL). Results are presented in Tables 29 and 30. As illustrated, both antisense compounds lowered total cholesterol levels; both lowered LDL; and both raised HDL. Further, the antisense compound conjugated to GalNAc₃-1 (ISIS 647535) was substantially more potent than the antisense compound lacking the GalNAc₃-1 Conjugate (ISIS 304801). An increase in HDL and a decrease in LDL levels is a cardiovascular beneficial effect of antisense inhibition of ApoC III.

TABLE 29
Effect of ASO treatment on HDL and LDL
cholesterol levels in transgenic mice

| | | | | Internu- | |
|---|---|---|---|---|---|
| | | Total | | cleoside | |
| | Dose | Cholesterol | 3′ | Linkage/ | SEQ |
| ASO | (μmol/kg) | (mg/dL) | Conjugate | Length | ID No. |
| PBS | 0 | 257 | — | — | |
| ISIS | 0.08 | 226 | None | PS/20 | 32 |
| 304801 | 0.75 | 164 | | | |
| | 2.25 | 110 | | | |
| | 6.75 | 82 | | | |
| ISIS | 0.08 | 230 | GalNAc₃-1 | PS/20 | 111 |
| 647535 | 0.75 | 82 | | | |
| | 2.25 | 86 | | | |
| | 6.75 | 99 | | | |

TABLE 30
Effect of ASO treatment on HDL and LDL
cholesterol levels in transgenic mice

| | | | | | Internu- | |
|---|---|---|---|---|---|---|
| | | | LDL | | cleoside | |
| | Dose | HDL | (mg/ | 3′ | Linkage/ | SEQ |
| ASO | (μmol/kg) | (mg/dL) | dL) | Conjugate | Length | ID No. |
| PBS | 0 | 17 | 28 | — | — | |
| ISIS | 0.08 | 17 | 23 | None | PS/20 | 32 |
| 304801 | 0.75 | 27 | 12 | | | |
| | 2.25 | 50 | 4 | | | |
| | 6.75 | 45 | 2 | | | |
| ISIS | 0.08 | 21 | 21 | GalNAc₃-1 | PS/20 | 111 |
| 647535 | 0.75 | 44 | 2 | | | |
| | 2.25 | 50 | 2 | | | |
| | 6.75 | 58 | 2 | | | |

### Pharmacokinetics Analysis (PK)

The PK of the ASOs was also evaluated. Liver and kidney samples were minced and extracted using standard protocols. Samples were analyzed on MSD1 utilizing IP-HPLC-MS. The tissue level (μg/g) of full-length ISIS 304801 and 647535 was measured and the results are provided in Table 31. As illustrated, liver concentrations of total full-length antisense compounds were similar for the two antisense compounds. Thus, even though the GalNAc₃-1 conjugated antisense compound is more active in the liver (as demonstrated by the RNA and protein data above), it is not present at substantially higher concentration in the liver. Indeed, the calculated EC₅₀ (provided in Table 31) confirms that the observed increase in potency of the conjugated compound cannot be entirely attributed to increased accumulation. This result suggests that the conjugate improved potency by a mechanism other than liver accumulation alone, possibly by improving the productive uptake of the antisense compound into cells.

The results also show that the concentration of GalNAc₃-1 conjugated antisense compound in the kidney is lower than that of antisense compound lacking the GalNAc conjugate. This has several beneficial therapeutic implications. For therapeutic indications where activity in the kidney is not sought, exposure to kidney risks kidney toxicity without corresponding benefit. Moreover, high concentration in kidney typically results in loss of compound to the urine resulting in faster clearance. Accordingly, for non-kidney targets, kidney accumulation is undesired. These data suggest that GalNAc₃-1 conjugation reduces kidney accumulation.

TABLE 31
PK analysis of ASO treatment in transgenic mice

| | Dose | Liver | Kidney | Liver EC₅₀ | 3′ | Internucleoside | SEQ |
|---|---|---|---|---|---|---|---|
| ASO | (μmol/kg) | (μg/g) | (μg/g) | (μg/g) | Conjugate | Linkage/Length | ID No. |
| ISIS | 0.1 | 5.2 | 2.1 | 53 | None | PS/20 | 32 |
| 304801 | 0.8 | 62.8 | 119.6 | | | | |
| | 2.3 | 142.3 | 191.5 | | | | |
| | 6.8 | 202.3 | 337.7 | | | | |
| ISIS | 0.1 | 3.8 | 0.7 | 3.8 | GalNAc₃-1 | PS/20 | 111 |
| 647535 | 0.8 | 72.7 | 34.3 | | | | |
| | 2.3 | 106.8 | 111.4 | | | | |
| | 6.8 | 237.2 | 179.3 | | | | |

Metabolites of ISIS 647535 were also identified and their masses were confirmed by high resolution mass spectrometry analysis. The cleavage sites and structures of the observed metabolites are shown below. The relative % of full length ASO was calculated using standard procedures and the results are presented in Table 32. The major metabolite of ISIS 647535 was full-length ASO lacking the entire conjugate (i.e. ISIS 304801), which results from cleavage at cleavage site A, shown below. Further, additional metabolites resulting from other cleavage sites were also observed. These results suggest that introducing other cleavable bonds such as esters, peptides, disulfides, phosphoramidates or acyl-hydrazones between the GalNAc₃-1 sugar and the ASO, which can be cleaved by enzymes inside the cell, or which may cleave in the reductive environment of the cytosol, or which are labile to the acidic pH inside endosomes and lysosomes, can also be useful.

TABLE 32
Observed full length metabolites of ISIS 647535

| Metabolite | ASO | Cleavage site | Relative % |
|---|---|---|---|
| 1 | ISIS 304801 | A | 36.1 |
| 2 | ISIS 304801 + dA | B | 10.5 |
| 3 | ISIS 647535 minus [3 GalNAc] | C | 16.1 |
| 4 | ISIS 647535 minus | D | 17.6 |
| | [3 GalNAc + 1 5-hydroxy- | | |
| | pentanoic acid tether] | | |
| 5 | ISIS 647535 minus | D | 9.9 |
| | [2 GalNAc + 2 5-hydroxy- | | |
| | pentanoic acid tether] | | |
| 6 | ISIS 647535 minus | D | 9.8 |
| | [3 GalNAc + 3 5-hydroxy- | | |
| | pentanoic acid tether] | | |

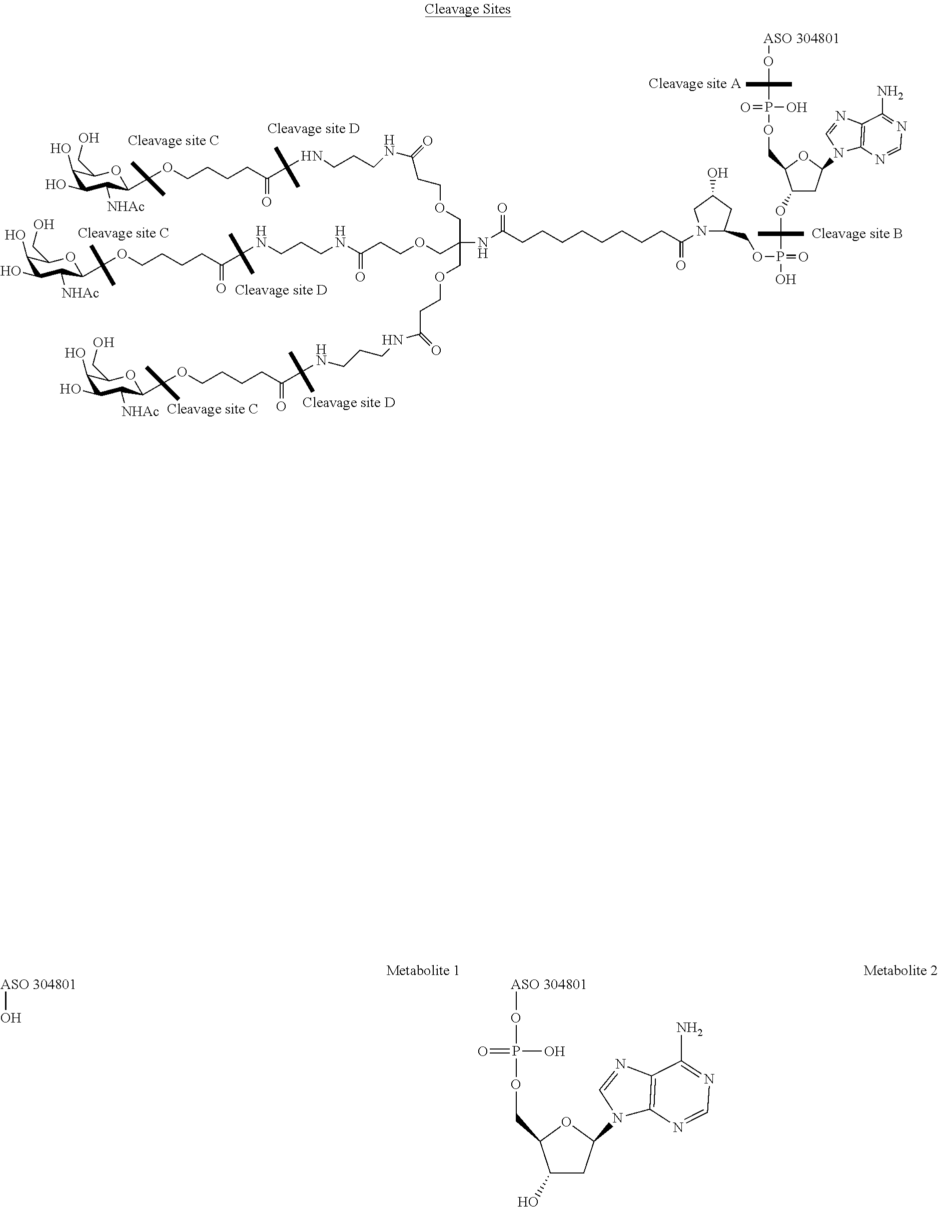

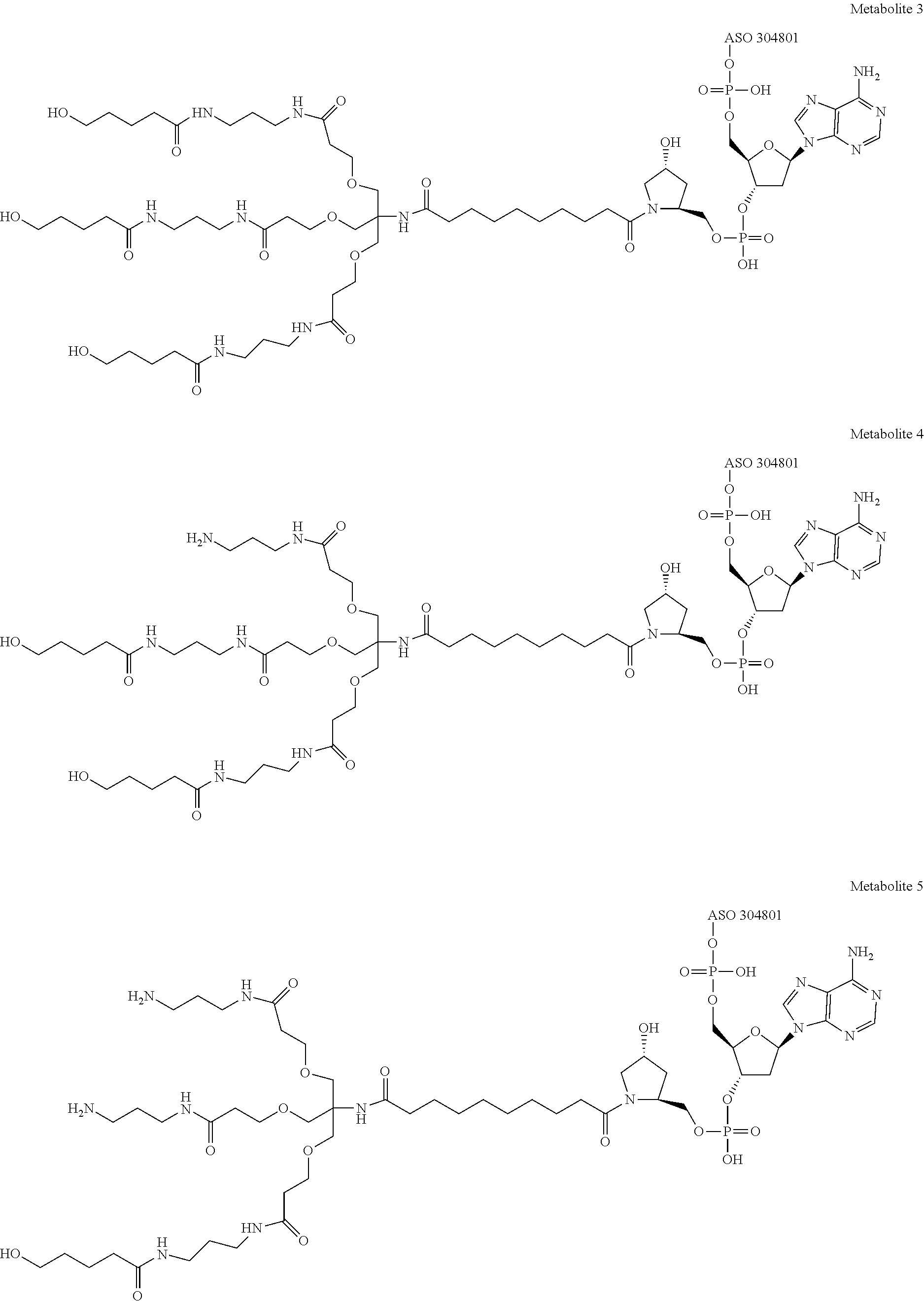

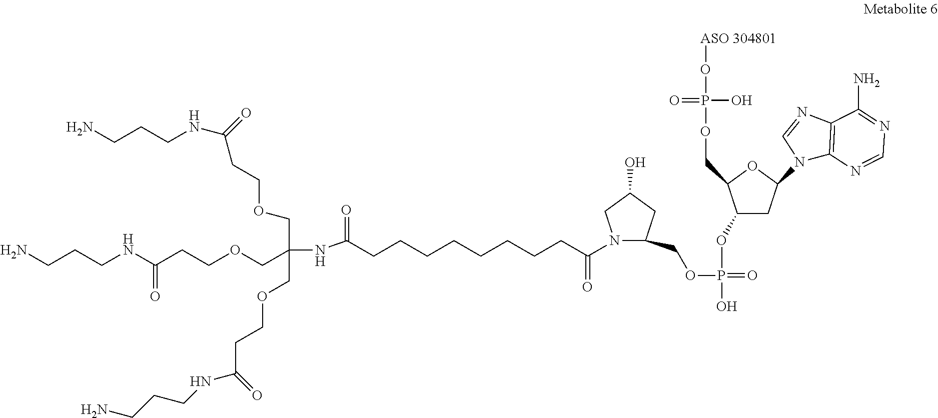

### Example 33: Dose-Dependent Study of Phosphodiester Linked GalNAc₃-7ₐ, β-Thio-GalNAc₃-7ₐ and MP-Triazole-GalNAc₃-7ₐ at the 5′ Terminus Targeting SRB-1 In Vivo

The conjugated oligonucleotides listed below were tested in a dose-dependent study for antisense inhibition of SRB-1 in mice. The unconjugated parent oligonucleotide ISIS 353382 was included in the study for comparison. The study compared the effect of GalNAc₃-7ₐ and two sugar modified GalNAc₃-7ₐ conjugate groups (β-thio-GalNAc₃-7ₐ, also referred to herein as GalNAc₃-35ₐ, structure shown in compound 115, wherein n=6 in Example 19; and MP-Triazole-GalNAc₃-7ₐ, also referred to herein as GalNAc₃-33ₐ structure shown in compound 148, wherein n=6, in Example 23) wherein each of the sugar modified oligonucleotides were tested with and without a deoxyadenosine (A_{d}) cleavable moiety.

| ISIS #/Seq | |
|---|---|
| Id No. | Sequence 5′-3′ |
| 353382/147 | Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds} |
| | Tₑₛ^{m}Cₑₛ^{m}CₑₛTₑₛTₑ |
| 666981/141 | GalNAc3-7a_{o′}A_{do}Gₑₛ^{m}CₑₛTₑₛTₑₛ^{m}CₑₛA_{ds}G_{ds}T_{ds}^{m}C_{ds}A_{ds} |
| | T_{ds}G_{ds}A_{ds}^{m}C_{ds}T_{ds}Tₑₛ^{m}Cₑₛ^{m}CₑₛTₑₛTₑ |
| 709049/141 | β-thio-GalNAc₃-7ₐ_{o′}A_{do}Gₑₛ^{m}CₑₛTₑₛTₑₛ^{m}CₑₛA_{ds}G_{ds} |
| | T_{ds}^{m}C_{ds}A_{ds}T_{ds}G_{ds}A_{ds}^{m}C_{ds}T_{ds}Tₑₛ^{m}Cₑₛ^{m}CₑₛTₑₛTₑ |
| 720810/147 | β-thio-GalNAc₃-7ₐ_{o′}Gₑₛ^{m}CₑₛTₑₛTₑₛ^{m}CₑₛA_{ds}G_{ds}T_{ds}^{m}C_{ds} |
| | A_{ds}T_{ds}G_{ds}A_{ds}^{m}C_{ds}T_{ds}Tₑₛ^{m}Cₑₛ^{m}CₑₛTₑₛTₑ |
| 721455/141 | C6-MP-Triazole-GalNAc₃-7ₐ_{o′}A_{do}Gₑₛ^{m}CₑₛTₑₛ |
| | Tₑₛ^{m}CₑₛA_{ds}G_{ds}T_{ds}^{m}C_{ds}A_{ds}T_{ds}G_{ds}A_{ds}^{m}C_{ds}T_{ds}Tₑₛ^{m}Cₑₛ^{m}Cₑₛ |
| | TₑₛTₑ |
| 721456/147 | C6-MP-Triazole-GalNAC₃-7ₐ_{o′}Gₑₛ^{m}CₑₛTₑₛTₑₛ^{m}Cₑₛ |
| | A_{ds}G_{ds}T_{ds}^{m}C_{ds}A_{ds}T_{ds}G_{ds}A_{ds}^{m}C_{ds}T_{ds}Tₑₛ^{m}Cₑₛ^{m}CₑₛTₑₛTₑ |

Capital letters indicate the nucleobase for each nucleoside and ^{m}C indicates a 5-methyl cytosine. Subscripts: “e” indicates a 2′-O—(CH₂)₃—OCH₃ (MOE) modified nucleoside; “d” indicates a β-D-2′-deoxyribonucleoside; “s” indicates a phosphorothioate internucleoside linkage (PS); “o” indicates a phosphodiester internucleoside linkage (PO); and “o′” indicates —O—P(═O)(OH)—. Conjugate groups are underlined.

### Treatment

Six week old male Balb/c mice (Jackson Laboratory, Bar Harbor, ME) were injected subcutaneously once at the dosage shown below with ISIS 353382, 666981, 709049, 720810, 721455, 721456 or with PBS treated control. Each treatment group consisted of 4 animals. The mice were sacrificed 72 hours following administration to determine the liver SRB-1 mRNA levels using real-time PCR and RIBOGREEN RNA quantification reagent (Molecular Probes, Inc. Eugene, OR) according to standard protocols. SRB-1 mRNA levels were determined relative to total RNA (using Ribogreen), prior to normalization to PBS-treated control. The results below are presented as the average percent of SRB-1 mRNA levels for each treatment group, normalized to PBS-treated control and is denoted as “% PBS”. The ED₅₀S were measured using similar methods as described previously and are presented below. The ED₅₀s listed in Table 33 below were calculated by plotting the concentrations of oligonucleotides used versus the percent inhibition of SRB-1 mRNA expression achieved at each concentration, and noting the concentration of oligonucleotide at which 5000 inhibition of SRB-1 mRNA expression was achieved compared to the control.

TABLE 33
ASOs containing GalNAc₃-7 w/wo
modified sugars targeting SRB-1

| ISIS | Dosage | SRB-1 mRNA | ED₅₀ | | SEQ ID |
|---|---|---|---|---|---|
| No. | (mg/kg) | levels (% PBS) | mg/kg | 5′-Conjugate | No. |
| PBS | 0 | 100 | — | — | — |
| 353382 | 3 | 107 | 27.2 | No conjugate | 147 |
| | 10 | 80 | | | |
| | 30 | 48 | | | |
| 666981 | 0.5 | 99 | 3.4 | GalNAc₃-7a with | 141 |
| | 1.5 | 71 | | dA | |
| | 5 | 35 | | | |
| | 15 | 21 | | | |
| 709049 | 0.5 | 87 | 3.1 | β-thio GalNAc₃- | 141 |
| | 1.5 | 66 | | 7a with dA | |
| | 5 | 38 | | | |
| | 15 | 18 | | | |
| 720810 | 0.5 | 80 | 3.3 | β-thio GalNAc₃- | 147 |
| | 1.5 | 66 | | 7a without dA | |
| | 5 | 43 | | | |
| | 15 | 19 | | | |
| 721455 | 0.5 | 90 | 4.7 | MP-Triazole | 141 |
| | 1.5 | 72 | | GalNAc₃-7a | |
| | 5 | 47 | | with dA | |
| | 15 | 29 | | | |
| 721456 | 0.5 | 85 | 3.7 | MP-Triazole | 147 |
| | 1.5 | 64 | | GalNAc₃-7a | |
| | 5 | 44 | | without dA | |
| | 15 | 27 | | | |

As illustrated in Table 33, treatment with antisense oligonucleotides lowered SRB-1 mRNA levels in a dose-dependent manner. Indeed, the antisense oligonucleotides comprising the phosphodiester linked conjugate groups showed substantial improvement in potency compared to the unconjugated antisense oligonucleotide (ISIS 353382).

The results of the body weights, liver transaminases, total bilirubin, and BUN measurements were all essentially unaffected by the oligonucleotides tested, indicating that the oligonucleotides were well tolerated.

### Example 34: Dose-Dependent Study of 5′ Phosphodiester Linked GalNAc₃-7ₐ, GalNAc₁-25ₐ, GalNAc₁-34ₐ, and α-Thio-GalNAc₃-7 Targeting SRB-1 In Vivo

The conjugated oligonucleotides listed below were tested in a dose-dependent study for antisense inhibition of SRB-1 in mice. The unconjugated parent oligonucleotide ISIS 353382 was included in the study for comparison. The study included a comparison of the effect of GalNAc₃-7ₐ (structure shown in Example 2), GalNAc₁-25ₐ (structure shown in Example 11) and GalNAc₁-34ₐ (structure shown in compound 153ₐ wherein n=6 in Example 24). These conjugate groups were attached directly to the parent antisense oligonucleotide without an A_{d} cleavable moiety. The study also included a comparison of the effect of GalNAc₃-7ₐ and α-thio-GalNAc₃-7ₐ (also referred to herein as GalNAc₃-36ₐ, structure shown in compound 120, wherein n=6, in Example 20). These conjugate groups were attached to the parent antisense oligonucleotide with an A_{d} cleavable moiety.

| ISIS #/Seq | |
|---|---|
| Id No. | Sequence 5′-3′ |
| 353382/142 | Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds} |
| | Tₑₛ^{m}Cₑₛ^{m}CₑₛTₑₛTₑ |
| 702489/142 | GalNAc₃-7ₐ_{o′}Gₑₛ^{m}CₑₛTₑₛTₑₛ^{m}CₑₛA_{ds}G_{ds}T_{ds}^{m}C_{ds}A_{ds}T_{ds} |
| | G_{ds}A_{ds}^{m}C_{ds}T_{ds}Tₑₛ^{m}Cₑₛ^{m}CₑₛTₑₛTₑ |
| 711462/142 | GalNAc₁-25ₐ_{o′}Gₑₛ^{m}CₑₛTₑₛTₑₛ^{m}CₑₛA_{ds}G_{ds}T_{ds}^{m}C_{ds}A_{ds}T_{ds} |
| | G_{ds}A_{ds}^{m}C_{ds}T_{ds}Tₑₛ^{m}Cₑₛ^{m}CₑₛTₑₛT |
| 727852/142 | GalNAc₁-34ₐ_{o′}Gₑₛ^{m}CₑₛTₑₛTₑₛ^{m}CₑₛA_{ds}G_{ds}T_{ds}^{m}C_{ds}A_{ds}T_{ds} |
| | G_{ds}A_{ds}^{m}C_{ds}T_{ds}Tₑₛ^{m}Cₑₛ^{m}CₑₛTₑₛT |
| 666981/141 | GalNAc₃-7ₐ_{o′}A_{do}Gₑₛ^{m}CₑₛTₑₛTₑₛ^{m}CₑₛA_{ds}G_{ds}T_{ds}^{m}C_{ds}A_{ds} |
| | T_{ds}G_{ds}A_{ds}^{m}C_{ds}T_{ds}Tₑₛ^{m}Cₑₛ^{m}CₑₛTₑₛTₑ |
| 720333/141 | α-thio-GalNAc₃-7ₐ_{o′}A_{do}Gₑₛ^{m}CₑₛTₑₛTₑₛ^{m}CₑₛA_{ds}G_{ds} |
| | T_{ds}^{m}C_{ds}A_{ds}T_{ds}G_{ds}A_{ds}^{m}C_{ds}T_{ds}Tₑₛ^{m}Cₑₛ^{m}CₑₛTₑₛTₑ |

Capital letters indicate the nucleobase for each nucleoside and ^{m}C indicates a 5-methyl cytosine. Subscripts: “e” indicates a 2′-O—(CH₂)₃—OCH₃ (MOE) modified nucleoside; “d” indicates a β-D-2′-deoxyribonucleoside; “s” indicates a phosphorothioate internucleoside linkage (PS); “o” indicates a phosphodiester internucleoside linkage (PO); and “o′” indicates —O—P(═O)(OH)—. Conjugate groups are underlined.

### Treatment

Six week old male Balb/c mice (Jackson Laboratory, Bar Harbor, ME) were injected subcutaneously with the ASOs listed below twice a week for 3 weeks at the dosage shown or with PBS. Each treatment group consisted of 4 animals. The mice were sacrificed 72 hours following administration to determine the liver SRB-1 mRNA levels using real-time PCR and RIBOGREEN® RNA quantification reagent (Molecular Probes, Inc. Eugene, OR) according to standard protocols. SRB-1 mRNA levels were determined relative to total RNA (using RIBOGREEN®), prior to normalization to PBS-treated control. The results below are presented as the average percent of SRB-1 mRNA levels for each treatment group. The data was normalized to PBS-treated control and is denoted as “% PBS”. The ED₅₀s listed in Table 34 were calculated by plotting the concentrations of oligonucleotides used versus the percent inhibition of SRB-1 mRNA expression achieved at each concentration, and noting the concentration of oligonucleotide at which 50% inhibition of SRB-1 mRNA expression was achieved compared to the control.

TABLE 34
ASOs containing mod/unmod
GalNAc₃-7ₐ/GalNAc₁-30ₐ targeting SRB-1

| ISIS | Dosage | SRB-1 mRNA | ED₅₀ | | SEQ ID |
|---|---|---|---|---|---|
| No. | (mg/kg) | levels (% PBS) | mg/kg | 5′-Conjugate | No. |
| PBS | 0 | 100 | — | — | — |
| 353382 | 3 | 72 | 20.7 | No conjugate | 147 |
| | 10 | 62 | | | |
| | 30 | 36 | | | |
| 666981 | 0.5 | 73 | 2.3 | GalNAc₃-7ₐ | 141 |
| | 1.5 | 55 | | with dA | |
| | 5 | 26 | | | |
| | 15 | 15 | | | |
| 720333 | 0.5 | 82 | 3.1 | α-thio | 141 |
| | 1.5 | 52 | | GalNAc₃-7ₐ | |
| | 5 | 31 | | with dA | |
| | 15 | 20 | | | |
| 702489 | 0.5 | 79 | 2.4 | GalNAc₃-7ₐ | 147 |
| | 1.5 | 65 | | without dA | |
| | 5 | 23 | | | |
| | 15 | 10 | | | |
| 711462 | 0.5 | 89 | 4.9 | GalNAc₁-25ₐ | 147 |
| | 1.5 | 75 | | without dA | |
| | 5 | 36 | | | |
| | 15 | 25 | | | |
| 727852 | 0.5 | 99 | 2.9 | GalNAc₁-34ₐ | 147 |
| | 1.5 | 70 | | without dA | |
| | 5 | 30 | | | |
| | 15 | 10 | | | |

As illustrated in Table 34, treatment with antisense oligonucleotides lowered SRB-1 mRNA levels in a dose-dependent manner. Indeed, the antisense oligonucleotides comprising the phosphodiester linked conjugate groups showed substantial improvement in potency compared to the unconjugated antisense oligonucleotide (ISIS 353382). It was unexpected that ISIS 727852 having a single modified GalNAc sugar provided equivalent activity compared to ISIS 702489 which comprises 3 unmodified GalNAc sugars.

The results of the body weights, liver transaminases, total bilirubin, and BUN measurements were all essentially unaffected by the oligonucleotides tested, indicating that the oligonucleotides were well tolerated.

### Example 35: Dose-Dependent Study of Modified ASOs Targeting APOC-III In Vivo

The compounds in the table below were designed to target mouse APOC-III.

TABLE 35
Modified ASOs targeting mouse APOC-III

| | | SEQ ID |
|---|---|---|
| Isis No. | Sequence | NO |
| 440670 | ^{m}Cₑₛ Aₑₛ Gₑₛ ^{m}Cₑₛ Tₑₛ T_{ds} T_{ds} A_{ds} T_{ds} T_{ds} A_{ds} G_{ds} G_{ds} G_{ds} A_{ds} ^{m}Cₑₛ Aₑₛ Gₑₛ ^{m}Cₑₛ Aₑ | 148 |
| 680772 | GalNAc₃-7_{a-o′}^{m}Cₑₛ Aₑ ₛGₑₛ ^{m}Cₑₛ Tₑₛ T_{ds} T_{ds} A_{ds} T_{ds} T_{ds} A_{ds} G_{ds} G_{ds} G_{ds} A_{ds} ^{m}Cₑₛ Aₑₛ | 148 |
| | Gₑₛ ^{m}Cₑₛ Aₑ | |
| 742119 | GalNAc₁-37ₐ₋ₒ_{′}^{m}Cₑₛ Aₑₛ Gₑₛ ^{m}Cₑₛ Tₑₛ T_{ds} T_{ds} A_{ds} T_{ds} T_{ds} A_{ds} G_{ds} G_{ds} G_{ds} A_{ds} ^{m}Cₑₛ | 148 |
| | Aₑₛ Gₑₛ ^{m}Cₑₛ Aₑ | |
| 742117 | GalNAc₁-34ₐ₋ₒ_{′}^{m}Cₑₛ Aₑₛ Gₑₛ ^{m}Cₑₛ Tₑₛ T_{ds} T_{ds} A_{ds} T_{ds} T_{ds} A_{ds} G_{ds} G_{ds} G_{ds} A_{ds} ^{m}Cₑₛ | 148 |
| | Aₑₛ Gₑₛ ^{m}Cₑₛ Aₑ | |
| 696846 | GalNAc₃-7ₐ₋ₒ_{′}^{m}Cₑₛ Aₑₒ Gₑₒ ^{m}Cₑₒ Tₑₒ T_{ds} T_{ds} A_{ds} T_{ds} T_{ds} A_{ds} G_{ds} G_{ds} G_{ds} A_{ds} ^{m}Cₑₒ | 148 |
| | Aₑₒ Gₑₛ ^{m}Cₑₛ Aₑ | |
| 742120 | GalNAc₁-37ₐ₋_{o′}^{m}Cₑₛ Aₑₒ Gₑₒ ^{m}Cₑₒ Tₑₒ T_{ds} T_{ds} A_{ds} T_{ds} T_{ds} A_{ds} G_{ds} G_{ds} G_{ds} A_{ds} ^{m}Cₑₒ | 148 |
| | Aₑₒ Gₑₛ ^{m}Cₑₛ Aₑ | |
| 742121 | GalNAc₁-34ₐ₋ₒ_{′}^{m}Cₑₛ Aₑₒ Gₑₒ ^{m}Cₑₒ Tₑₒ T_{ds} T_{ds} A_{ds} T_{ds} T_{ds} A_{ds} G_{ds} G_{ds} G_{ds} A_{ds} ^{m}Cₑₒ | 148 |
| | Aₑₒ Gₑₛ ^{m}Cₑₛ Aₑ | |

The structure of GalNAc₁-37ₐ is shown below:

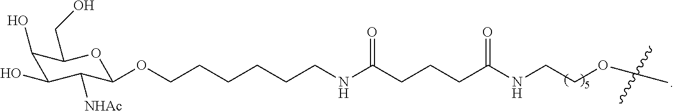

C57/BL6 mice were injected subcutaneously once with the ASOs listed above at the dosage shown or with PBS. Each treatment group consisted of 4 animals. The mice were sacrificed 72 hours following administration to determine the liver APOC-III mRNA levels using real-time PCR and RIBOGREEN® RNA quantification reagent (Molecular Probes, Inc. Eugene, OR) according to standard protocols. APOC-III mRNA levels were determined relative to total RNA (using RIBOGREEN®), prior to normalization to PBS-treated control. The results below are presented as the average percent of APOC-III mRNA levels for each treatment group. The data was normalized to PBS-treated control and is denoted as “C % PBS”. ED₅₀'s were calculated using nonlinear regression. The results below illustrate that the ASOs comprising one, modified GalNAc sugar (GalNAc₁-34ₐ) were more potent than the ASOs comprising one, unmodified GalNAc sugar (GalNAc₁-37ₐ) and were nearly as potent as the ASOs comprising three, unmodified GalNAc sugars (GalNAc₃-7ₐ).

TABLE 36
Activity of modified ASOs targeting mouse APOC-III in vivo

| | | APOC-III | | | |
|---|---|---|---|---|---|
| | Dose | mRNA | ED₅₀ | Conjugate | SEQ ID |
| Isis No. | (mg/kg) | (% PBS) | (mg/kg) | group | NO. |
| 440670 | 2 | 86 | 25.5 | n/a | 148 |
| | 6 | 77 | | | |
| | 20 | 56 | | | |
| | 60 | 32 | | | |
| 680772 | 0.6 | 77 | 3.1 | GalNAc₃-7ₐ | 148 |
| | 2 | 64 | | | |
| | 6 | 32 | | | |
| | 20 | 19 | | | |
| 742119 | 0.6 | 89 | 5.3 | GalNAc₁-37ₐ | 148 |
| | 2 | 78 | | | |
| | 6 | 45 | | | |
| | 20 | 19 | | | |
| 742117 | 0.6 | 92 | 4.9 | GalNAc₁-34ₐ | 148 |
| | 2 | 72 | | | |
| | 6 | 35 | | | |
| | 20 | 30 | | | |
| 696846 | 0.6 | 77 | 1.3 | GalNAc₃-7ₐ | 148 |
| | 2 | 28 | | | |
| | 6 | 17 | | | |
| | 20 | 12 | | | |
| 742120 | 0.6 | 96 | 6.7 | GalNAc₁-37ₐ | 148 |
| | 2 | 86 | | | |
| | 6 | 52 | | | |
| | 20 | 19 | | | |
| 742121 | 0.6 | 88 | 3.4 | GalNAc₁-34ₐ | 148 |
| | 2 | 62 | | | |
| | 6 | 33 | | | |
| | 20 | 20 | | | |

### Example 36: Oligonucleotides Comprising at Least One Modified GalNAc

The following modified GalNAc sugars are conjugated to oligonucleotides. Each oligonucleotide comprises one, two, or three modified GalNAc sugars.

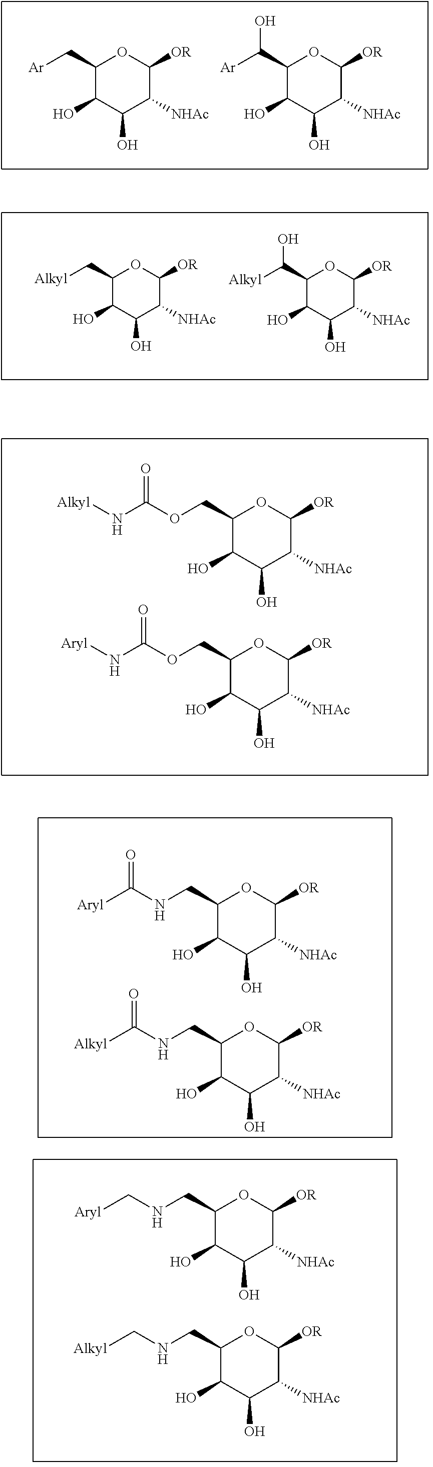

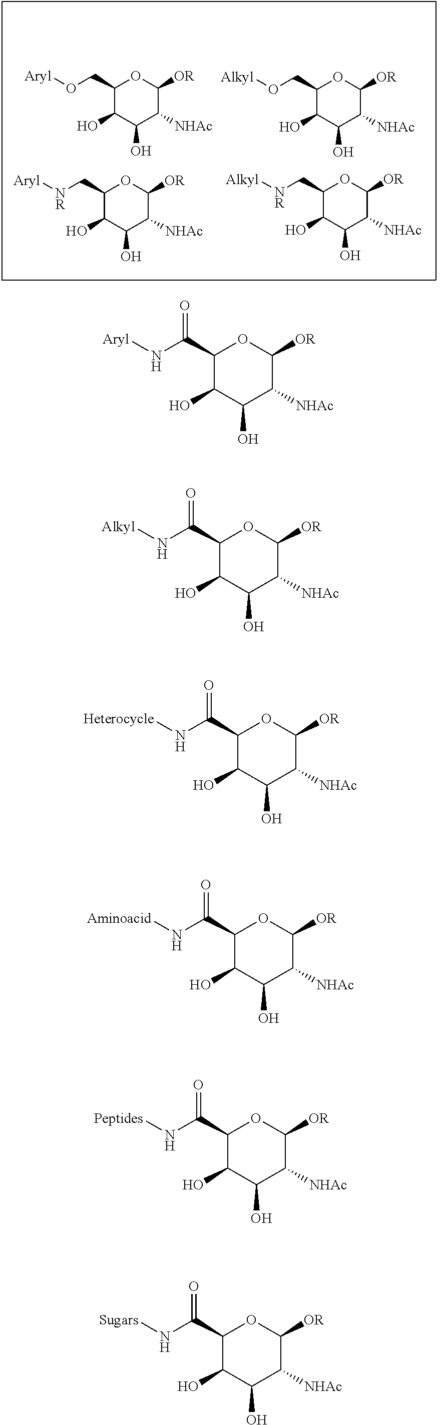

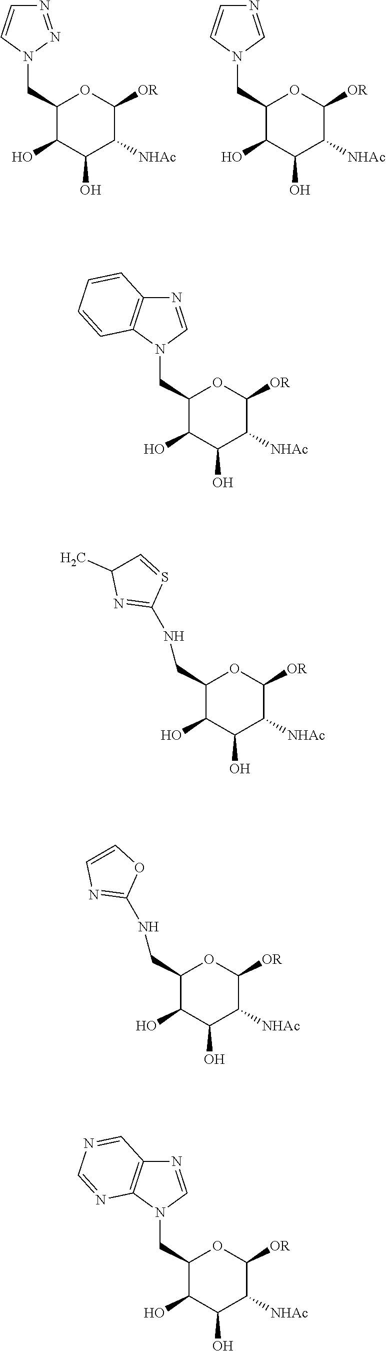

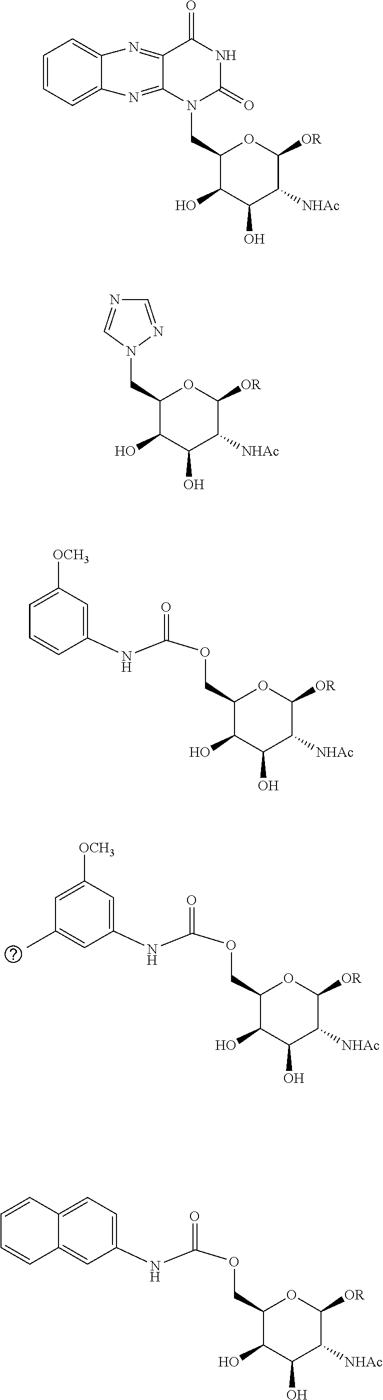

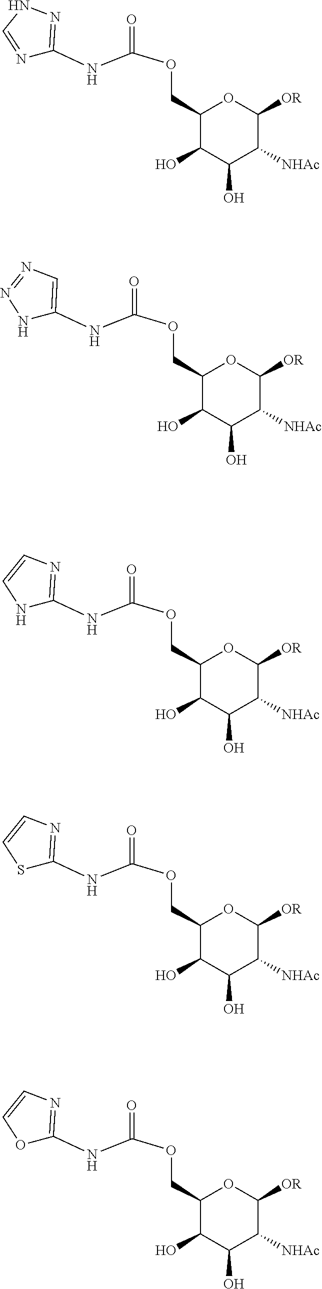

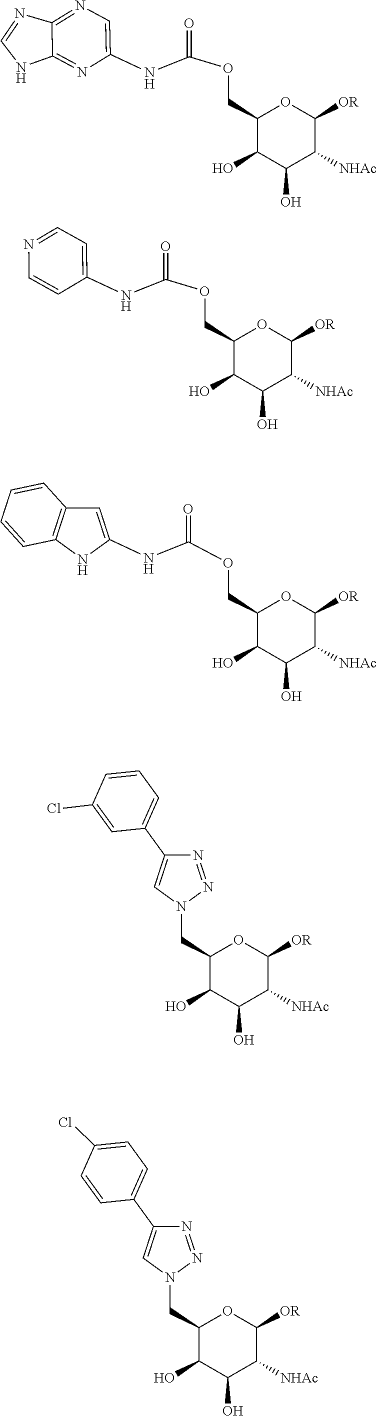

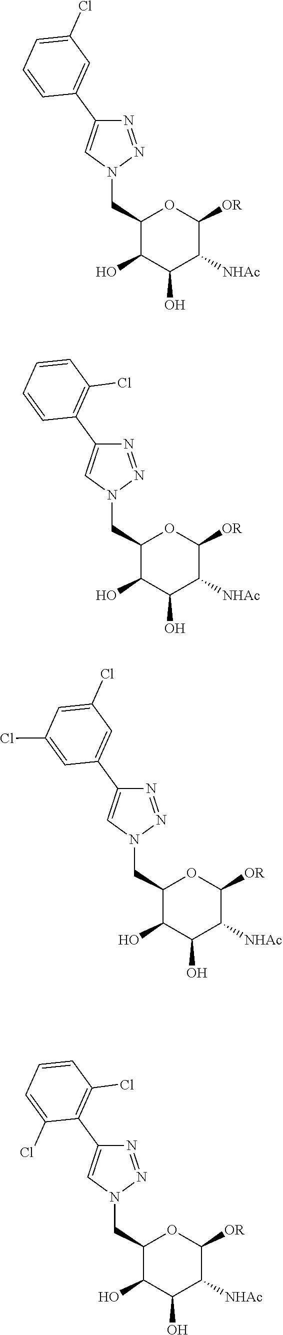

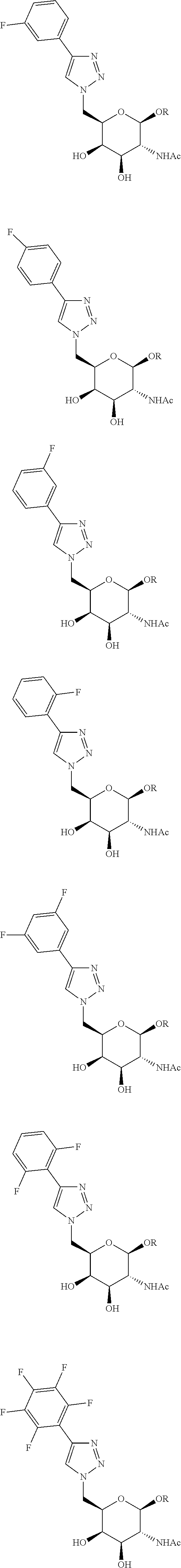

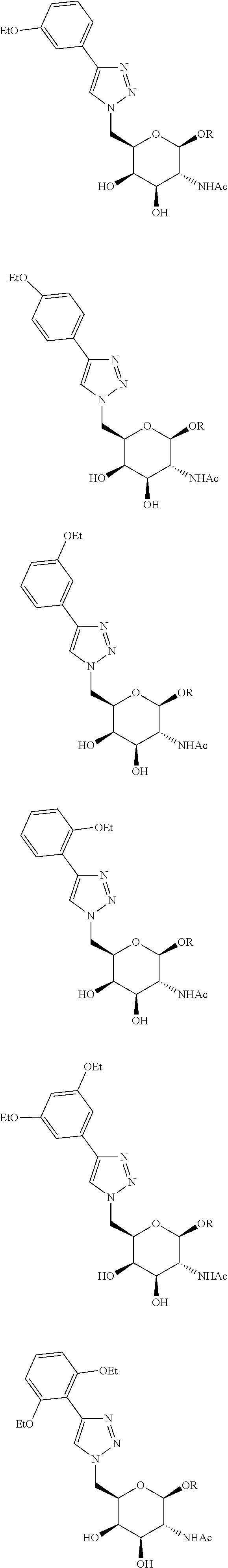

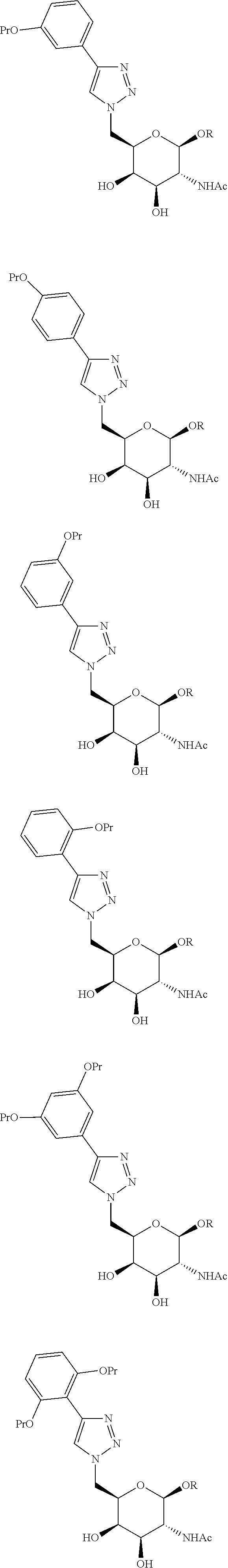

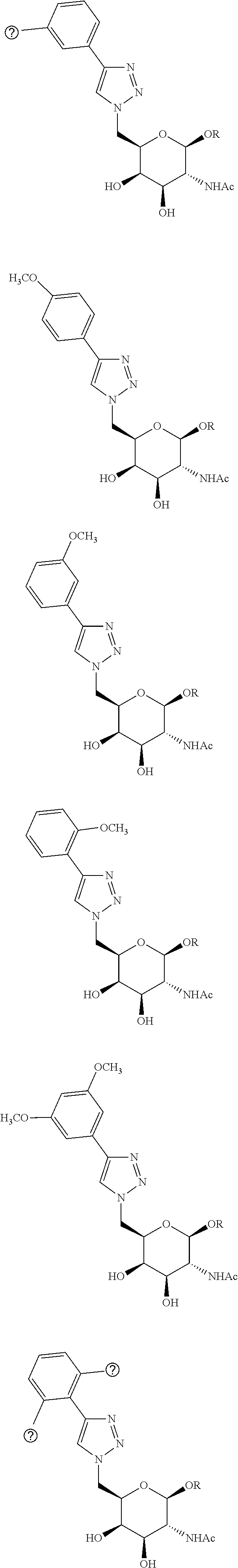

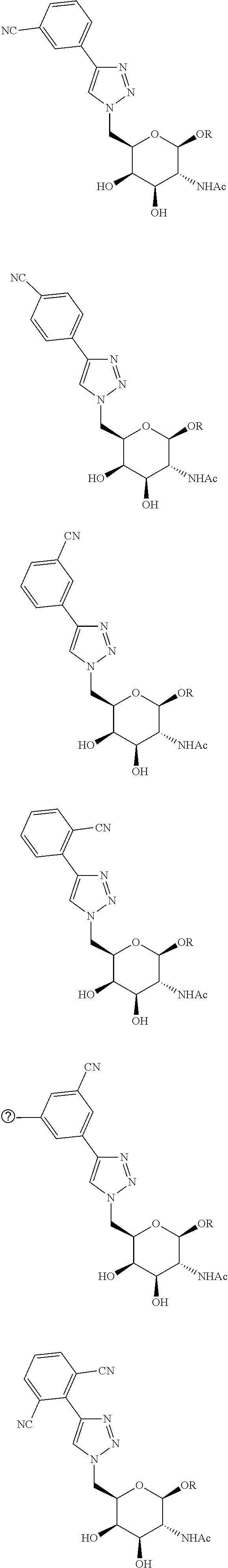

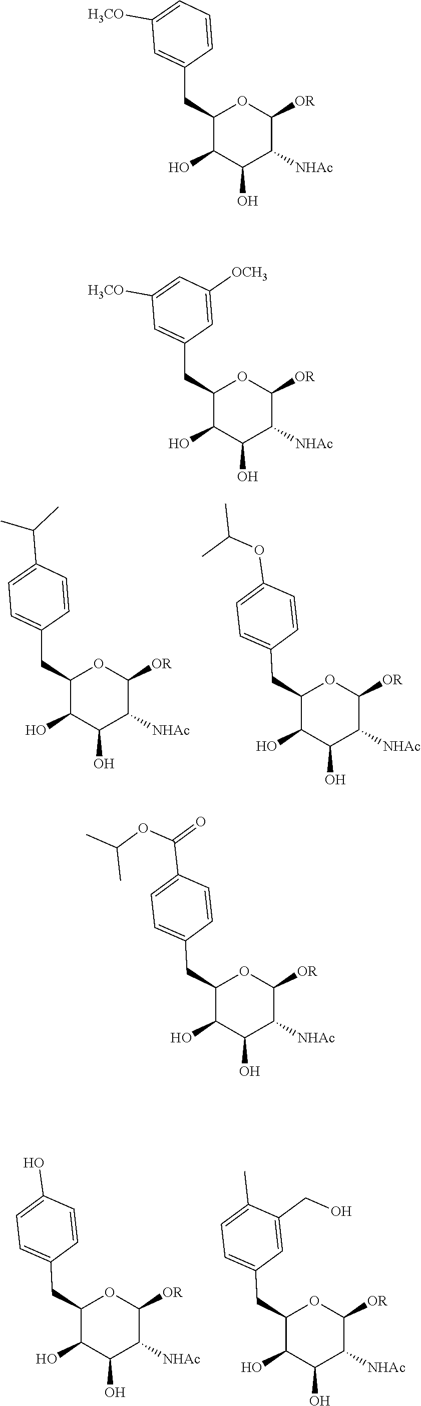

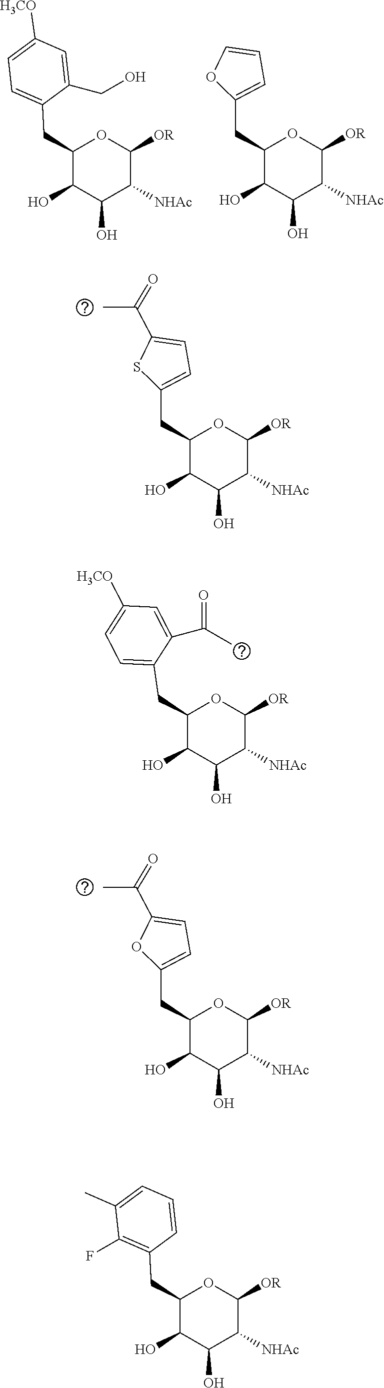

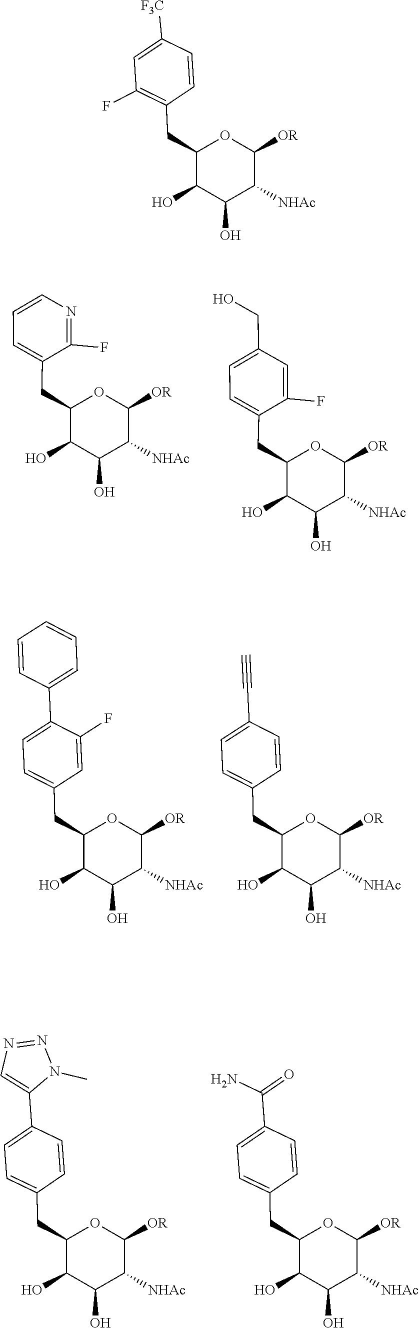

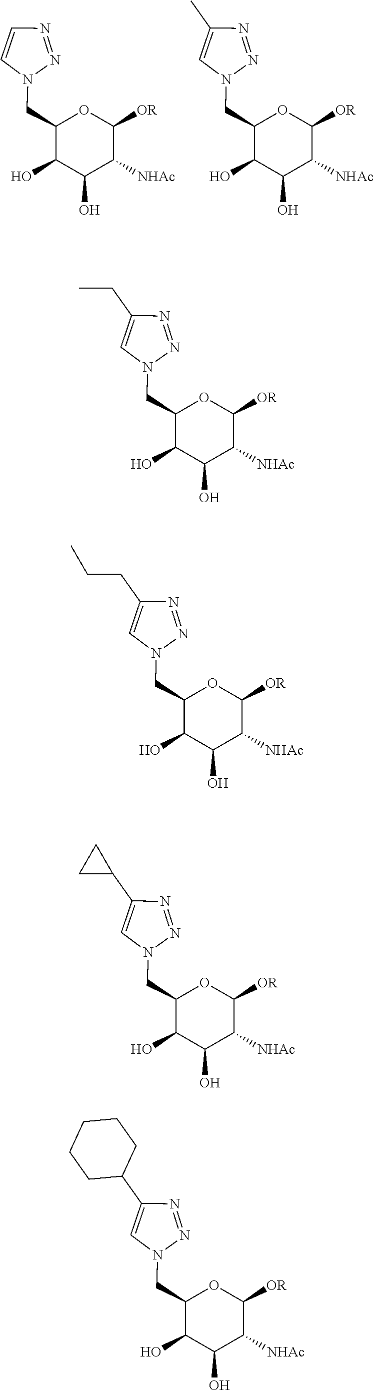

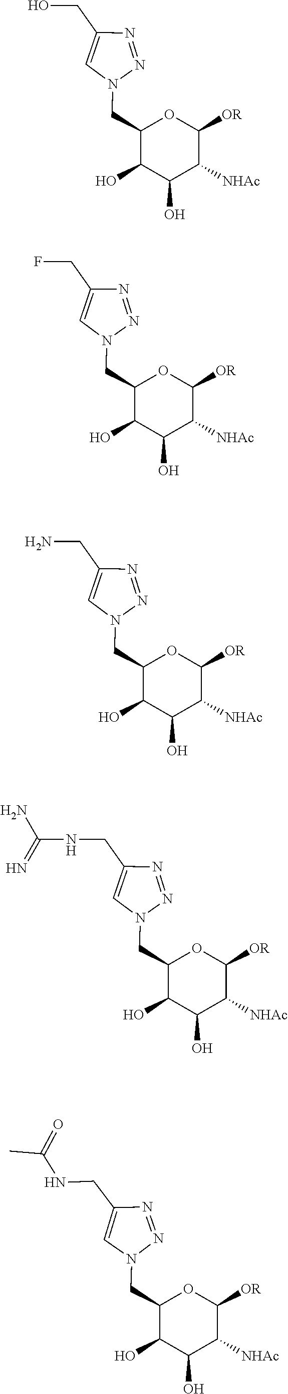

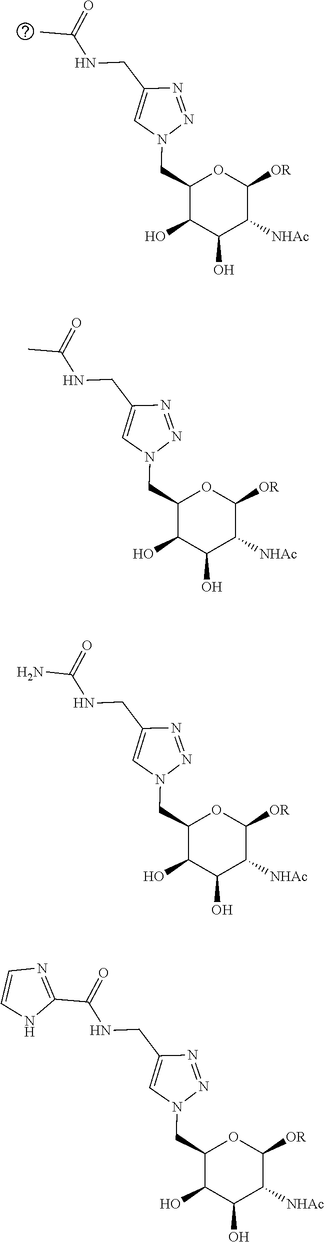

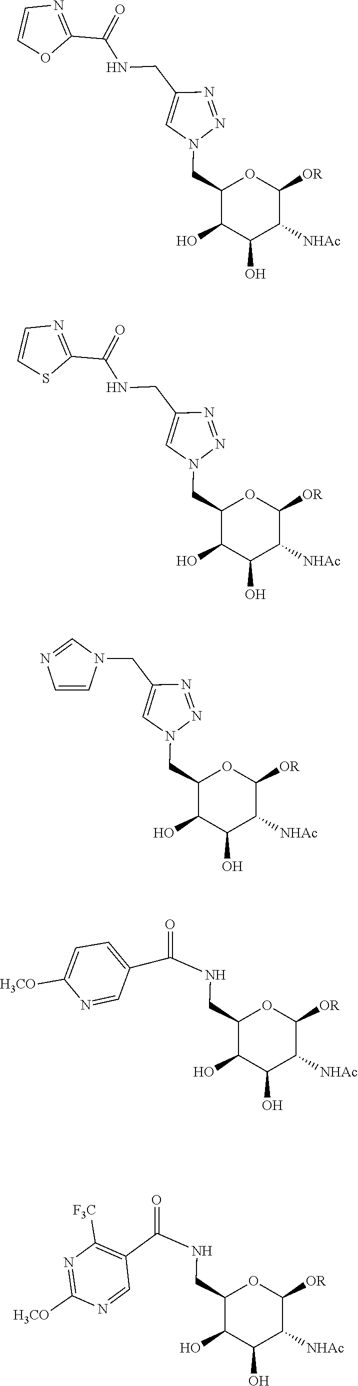

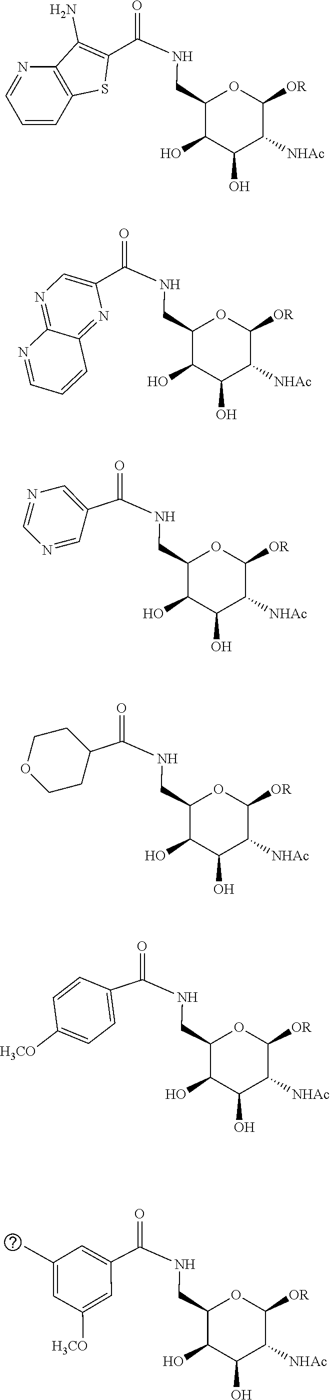

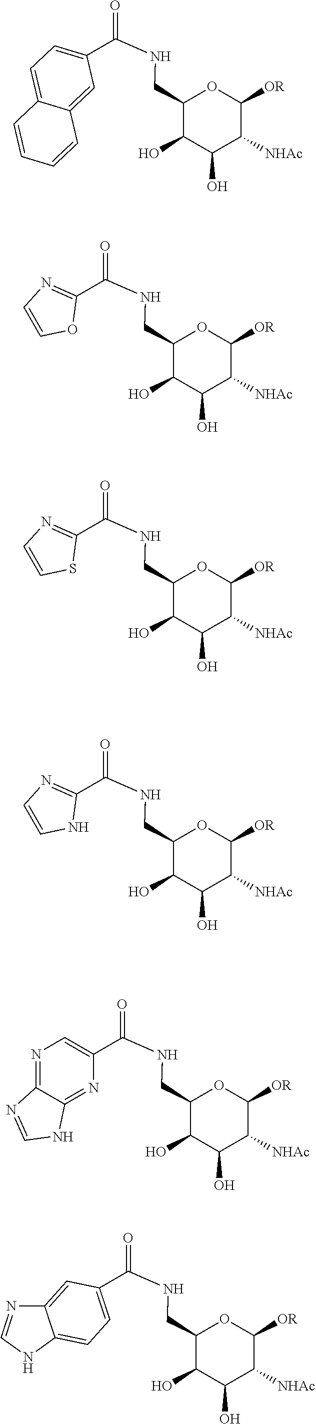

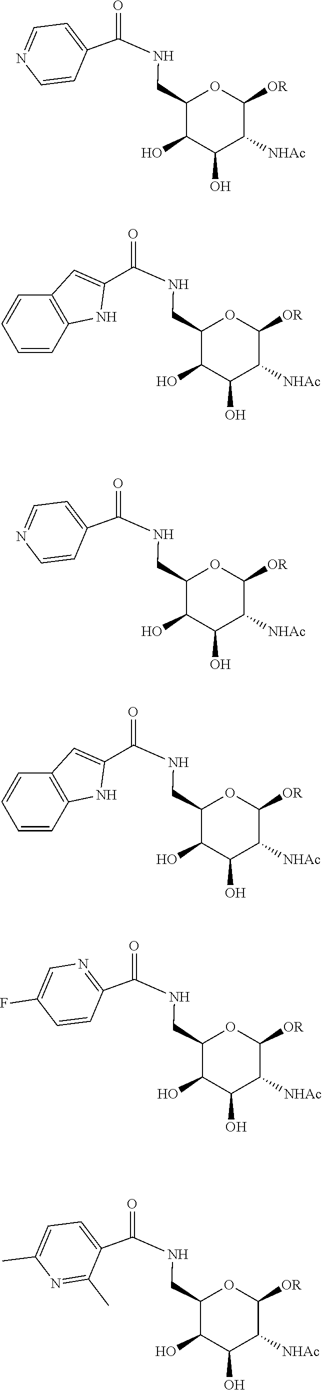

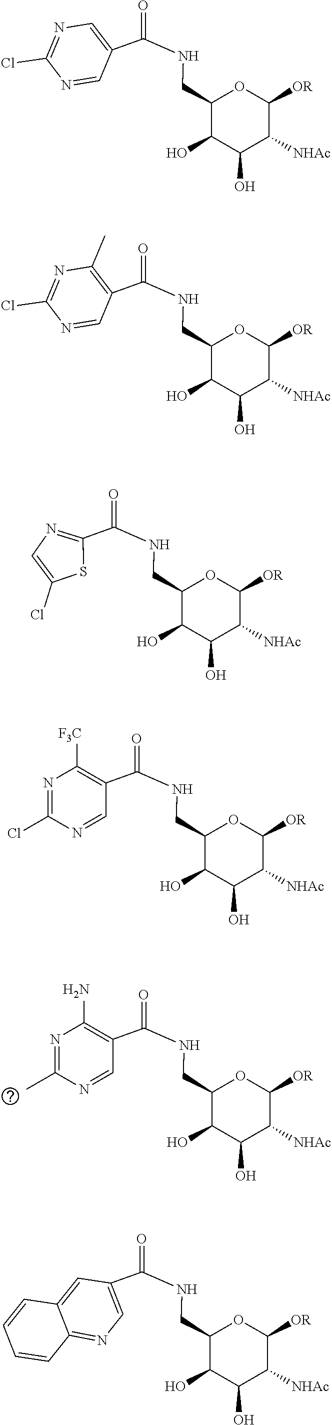

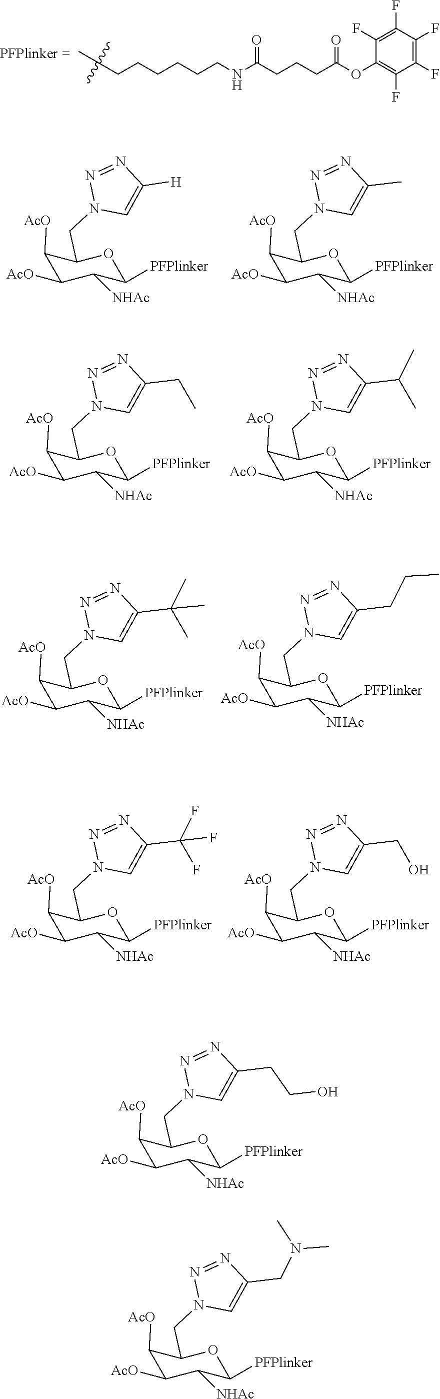

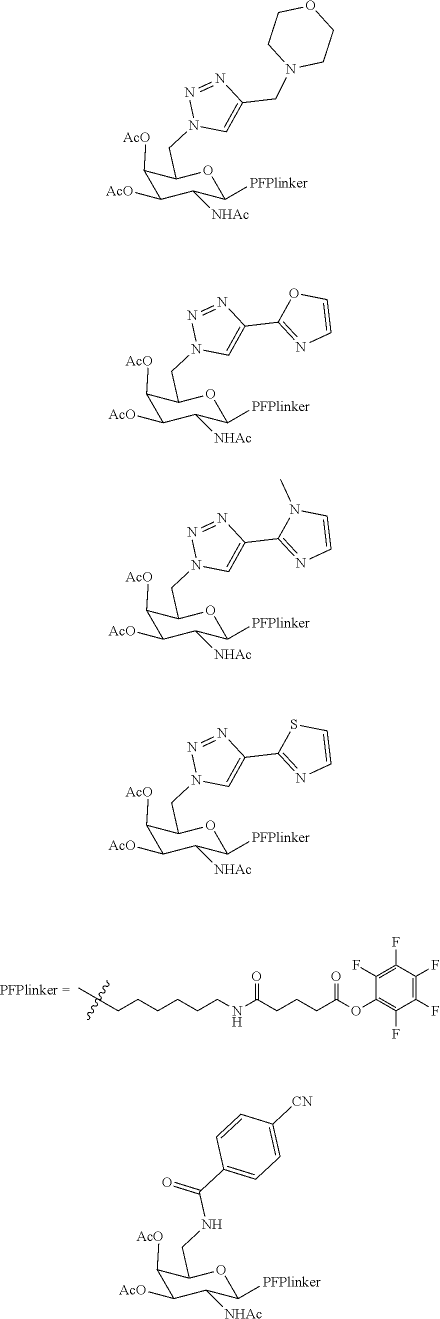

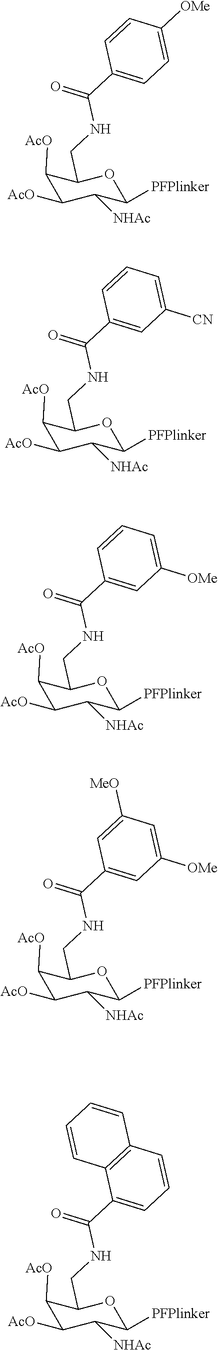

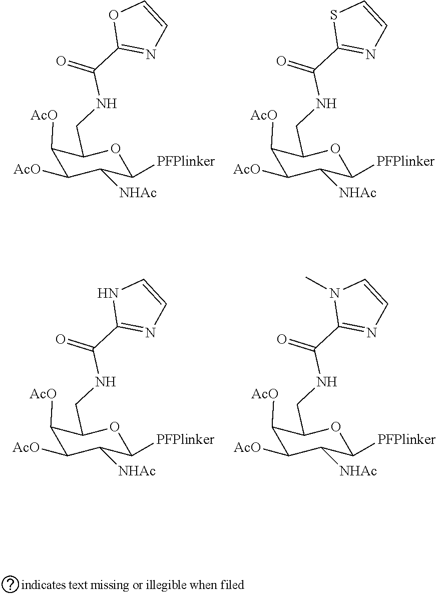

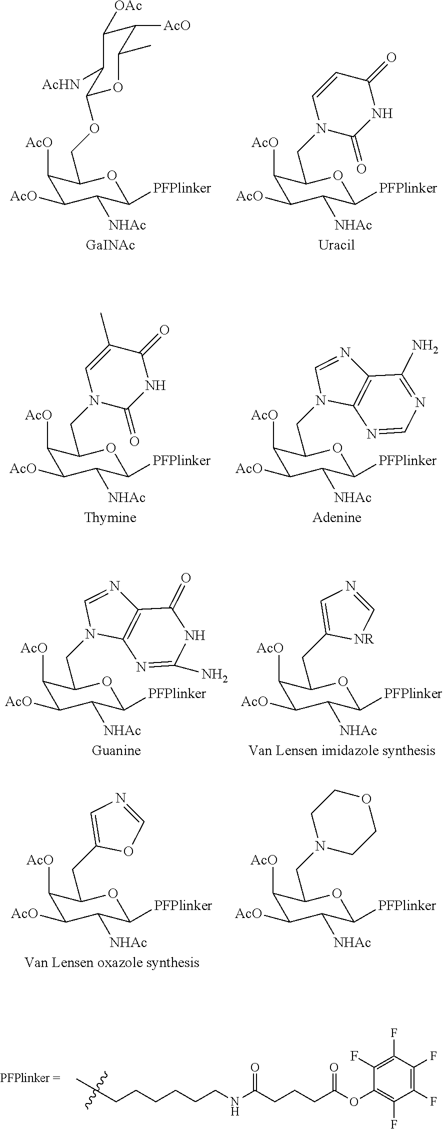

Certain modified GalNAc structures shown above are synthesized via the following scheme:

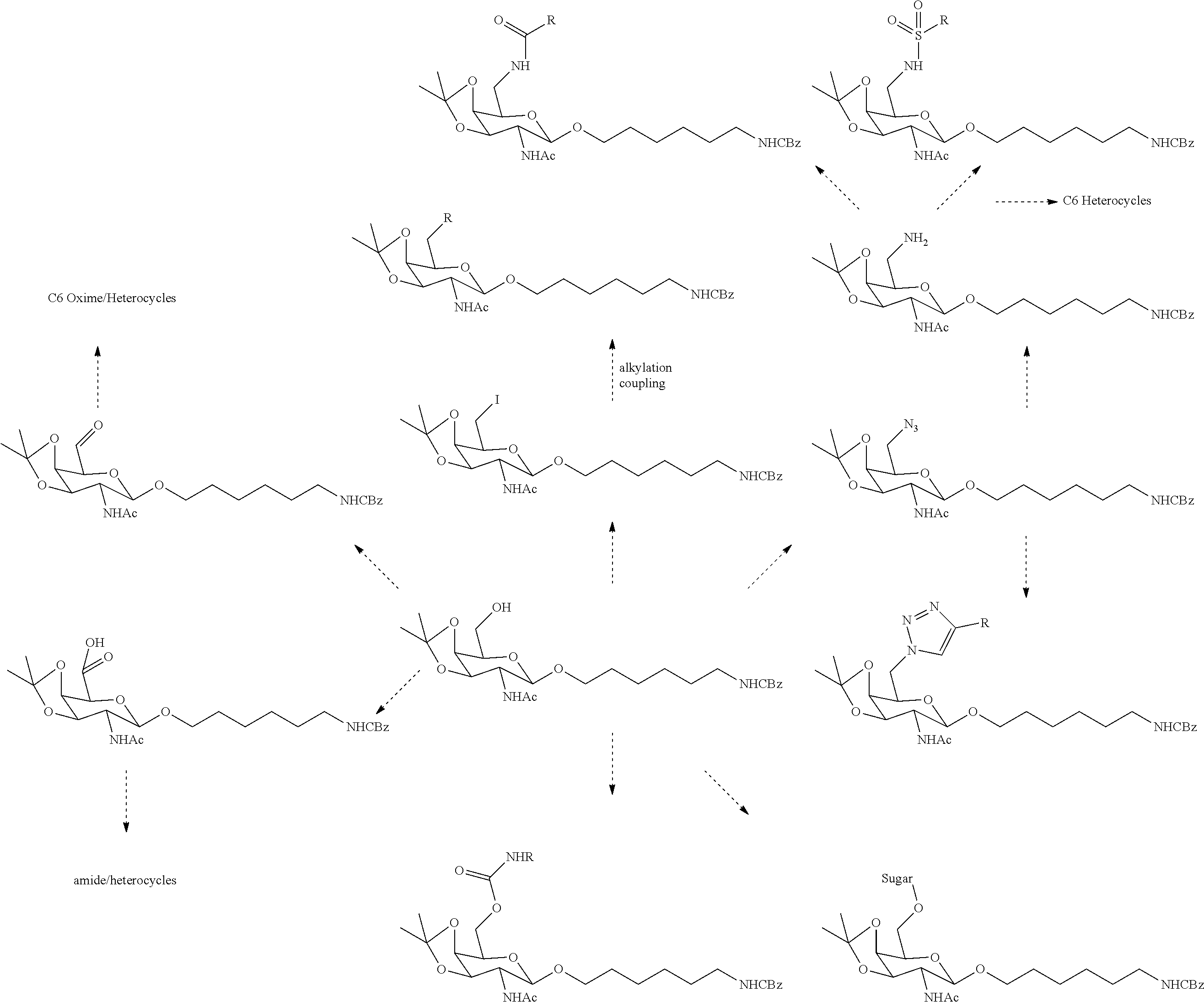

### Example 37: Dose-Dependent Study of Modified Oligonucleotides Targeting SRB1 In Vivo

The compounds in Table 37 were designed to target mouse SRB1. Wild type mice were injected subcutaneously once with a modified oligonucleotide listed in Table 37 at 0.2, 0.6, 2.0, or 6.0 mg/kg. A control group was injected subcutaneously with PBS. Each treatment group consisted of 2-4 animals. The mice were sacrificed 72 hours following oligonucleotide administration to determine the liver SRB1 mRNA levels using real-time PCR according to standard protocols. SRB1 mRNA levels were determined relative to total RNA (using RIBOGREEN®), prior to normalization to PBS treated control. The average percent of SRB1 mRNA levels for each treatment group relative to the average for the PBS treated group were used to calculate ED₅₀'s via nonlinear regression. The results below illustrate that the oligonucleotide comprising one, modified GalNAc sugar (GalNAc₁-34ₐ) was more potent than the oligonucleotide comprising one, unmodified GalNAc sugar (GalNAc₁-37ₐ).

TABLE 37
Activity of modified oligonucleotides targeting mouse SRB1

| Isis No. | Sequence | ED₅₀ (mg/kg) | SEQ ID NO. |
|---|---|---|---|
| 780123 | GalNAc₁-37_{a-o′}Tₖₛ ^{m}Cₖₛ A_{ds} G_{ds} T_{ds} ^{m}C_{ds} A_{ds} T_{ds} G_{ds} A_{ds} ^{m}C_{ds} T_{ds} Tₖₛ | 0.99 | 149 |
| | ^{m}Cₖ | | |
| 780121 | GalNAc₁-34_{a-o′}Tₖₛ ^{m}Cₖₛ A_{ds} G_{ds} T_{ds} ^{m}C_{ds} A_{ds} T_{ds} G_{ds} A_{ds} ^{m}C_{ds} T_{ds} Tₖₛ | 0.70 | 149 |
| | ^{m}Cₖ | | |

The structures of GalNAc₁-34ₐ and GalNAc₁-37ₐ are described above.

### Example 38: Single Dose Study of Modified Oligonucleotides Targeting SRB1 In Vivo

The compounds in Table 38 were designed to target mouse SRB1. Wild type mice were injected subcutaneously once with a modified oligonucleotide listed in Table 38 at 4.5 mg/kg. A control group was injected subcutaneously with PBS. Each treatment group consisted of 2-4 animals. The mice were sacrificed 72 hours following oligonucleotide administration to determine the liver SRB1 mRNA levels using real-time PCR according to standard protocols. SRB1 mRNA levels were determined relative to total RNA (using RIBOGREEN®), prior to normalization to PBS treated control. The results below are presented as the average percent of SRB1 mRNA levels for each treatment group relative to the average for the PBS treated group. The results below illustrate that the oligonucleotides comprising various modified GalNAc sugars decreased target mRNA levels and several oligonucleotides comprising various modified GalNAc sugars were more potent than the oligonucleotide comprising an unmodified GalNAc sugar (GalNAc₁-37ₐ).

TABLE 38
Activity of modified oligonucleotides targeting mouse SRB1

| | | SRB1 mRNA | SEQ |
|---|---|---|---|
| Isis No. | Sequence | (% PBS) | ID NO. |
| 736690 | GalNAc₁-37_{a-o′}Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ | 41 | 147 |
| | ^{m}Cₑₛ^{m}CₑₛTₑₛTₑ | | |
| 727852 | GalNAc₁-34_{a-o′} Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ | 35 | 147 |
| | ^{m}Cₑₛ^{m}CₑₛTₑₛTₑ | | |
| 748826 | GalNAc-38_{a-o′} Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ | 35 | 147 |
| | ^{m}Cₑₛ^{m}CₑₛTₑₛTₑ | | |
| 748828 | GalNAc₁-39_{a-o′} Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ | 35 | 147 |
| | ^{m}Cₑₛ^{m}CₑₛTₑₛTₑ | | |
| 750494 | GalNAc₁-40_{a-o′} Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ | 43 | 147 |
| | ^{m}Cₑₛ^{m}CₑₛTₑₛTₑ | | |
| 750493 | GalNAc₁41_{a-o′} Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ | 43 | 147 |
| | ^{m}Cₑₛ^{m}CₑₛTₑₛTₑ | | |
| 752377 | GalNAc₁-42_{a-o′} Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ | 38 | 147 |
| | ^{m}Cₑₛ^{m}CₑₛTₑₛTₑ | | |

The structures of GalNAc₁-34ₐ and GalNAc₁-37ₐ are described above. Compounds comprising GalNAc₁-38ₐ, GalNAc₁-39ₐ, GalNAc₁-40ₐ, GalNAc₁-42ₐ, and GalNAc₁-42ₐ were made using synthetic routes described herein, routes similar to those described herein, or reactions known in the art. The structures are shown below:

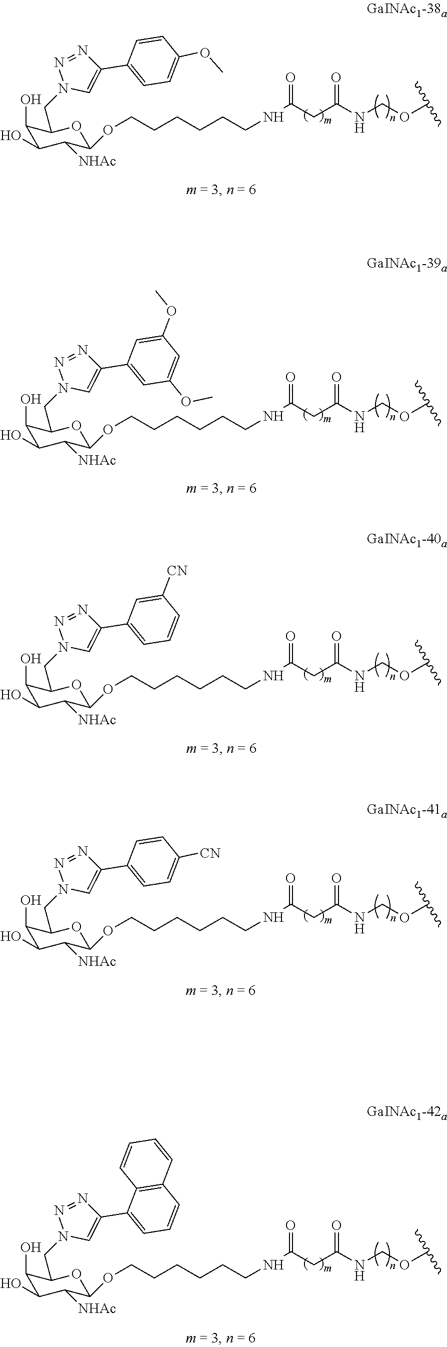

### Example 39: Single Dose Study of Modified Oligonucleotides Targeting SRB1 In Vivo

The compounds in Table 39 were designed to target mouse SRB1. Wild type mice were injected subcutaneously once with a modified oligonucleotide listed in Table 39 at 4.5 mg/kg. A control group was injected subcutaneously with PBS. Each treatment group consisted of 2-4 animals. The mice were sacrificed 72 hours following oligonucleotide administration to determine the liver SRB1 mRNA levels using real-time PCR according to standard protocols. SRB1 mRNA levels were determined relative to total RNA (using RIBOGREEN®), prior to normalization to PBS treated control. The results below are presented as the average percent of SRB1 mRNA levels for each treatment group relative to the average for the PBS treated group. The results below illustrate that the oligonucleotides comprising various modified GalNAc sugars decreased target mRNA levels.

TABLE 39
Activity of modified oligonucleotides targeting mouse SRB1

| | | SRB1 mRNA | SEQ |
|---|---|---|---|
| Isis No. | Sequence | (% PBS) | ID NO. |
| 801359 | GalNAc₁-43_{a-o′}Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ | 66 | 147 |
| | ^{m}Cₑₛ^{m}CₑₛTₑₛTₑ | | |
| 727852 | GalNAc₁-34_{a-o′} Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ | 53 | 147 |
| | ^{m}Cₑₛ^{m}CₑₛTₑₛTₑ | | |
| 801353 | GalNAc₁-44_{a-o′} Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ | 66 | 147 |
| | ^{m}Cₑₛ^{m}CₑₛTₑₛTₑ | | |
| 801360 | GalNAc₁-45_{a-o′} Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ | 67 | 147 |
| | ^{m}Cₑₛ^{m}CₑₛTₑₛTₑ | | |
| 801357 | GalNAc₁-46_{a-o′} Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ | 59 | 147 |
| | ^{m}Cₑₛ^{m}CₑₛTₑₛTₑ | | |
| 801358 | GalNAc₁-47_{a-o′} Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ | 54 | 147 |
| | ^{m}Cₑₛ^{m}CₑₛTₑₛTₑ | | |
| 801354 | GalNAc₁-48_{a-o′} Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ | 60 | 147 |
| | ^{m}Cₑₛ^{m}CₑₛTₑₛTₑ | | |

The structure of GalNAc₁-34ₐ is described above. Compounds comprising GalNAc₁-43ₐ, GalNAc₁-44ₐ, GalNAc₁-45ₐ, GalNAc₁-46ₐ, GalNAc₁-47ₐ, and GalNAc₁-48ₐ were made using synthetic routes described herein, routes similar to those described herein, or reactions known in the art. The structures are shown below:

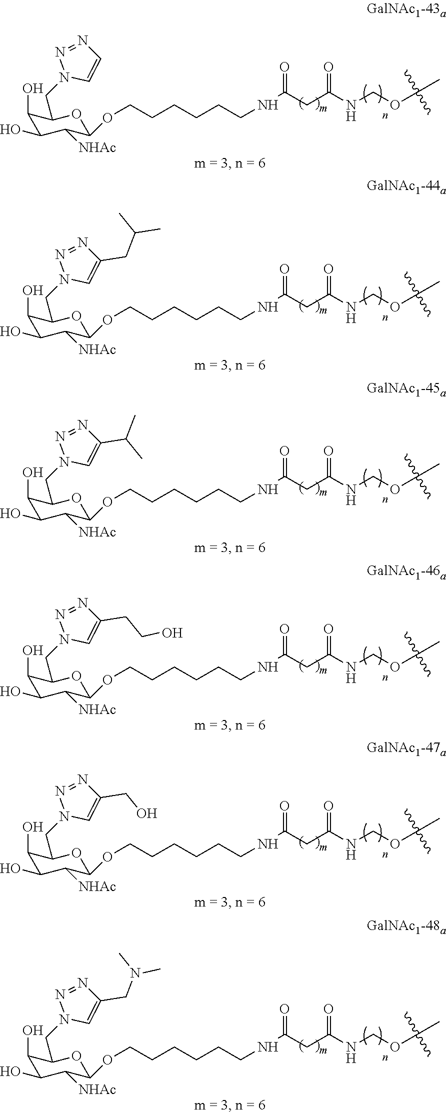

### Example 40: Single Dose Study of Modified Oligonucleotides Targeting SRB1 In Vivo

The compounds in Table 40 were designed to target mouse SRB1. Wild type mice were injected subcutaneously once with a modified oligonucleotide listed in Table 40 at 4.5 mg/kg. A control group was injected subcutaneously with PBS. Each treatment group consisted of 2-4 animals. The mice were sacrificed 72 hours following oligonucleotide administration to determine the liver SRB1 mRNA levels using real-time PCR according to standard protocols. SRB1 mRNA levels were determined relative to total RNA (using RIBOGREEN®), prior to normalization to PBS treated control. The results below are presented as the average percent of SRB1 mRNA levels for each treatment group relative to the average for the PBS treated group. The results below illustrate that the oligonucleotides comprising various modified GalNAc sugars decreased target mRNA levels and several oligonucleotides comprising various modified GalNAc sugars were more potent than the oligonucleotide comprising an unmodified GalNAc sugar (GalNAc₁-37ₐ).

TABLE 40
Activity of modified oligonucleotides targeting mouse SRB1

| | | SRB1 mRNA | SEQ |
|---|---|---|---|
| Isis No. | Sequence | (% PBS) | ID NO. |
| 736690 | GalNAc₁-37_{a-o′}Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ | 55 | 147 |
| | ^{m}Cₑₛ^{m}CₑₛTₑₛTₑ | | |
| 727852 | GalNAc₁-34_{a-o′} Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ | 42 | 147 |
| | ^{m}Cₑₛ^{m}CₑₛTₑₛTₑ | | |
| 761852 | GalNAc₁-49_{a-o′} Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ | 57 | 147 |
| | ^{m}Cₑₛ^{m}CₑₛTₑₛTₑ | | |
| 761853 | GalNAc₁-50_{a-o′} Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ | 54 | 147 |
| | ^{m}Cₑₛ^{m}CₑₛTₑₛTₑ | | |
| 761854 | GalNAc₁-51_{a-o′} Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ | 52 | 147 |
| | ^{m}Cₑₛ^{m}CₑₛTₑₛTₑ | | |

The structures of GalNAc₁-34ₐ and GalNAc₁-37ₐ are described above. Compounds comprising GalNAc₁-49ₐ, GalNAc₁-50ₐ, and GalNAc₁-51ₐ were made using synthetic routes described herein, routes similar to those described herein, or reactions known in the art. The structures are shown below:

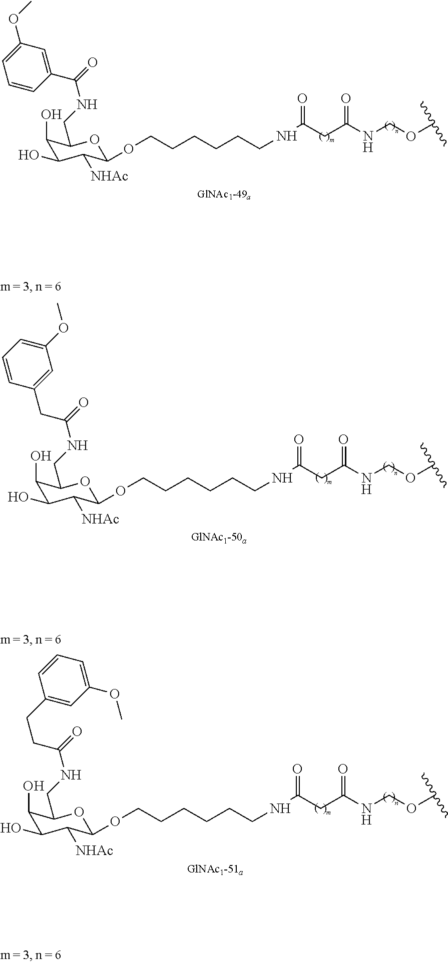 [SMILES: *OCNC(=O)CC(=O)NCCCCCCOC1OC(CNC(=O)c2cccc(OC)c2)C(O)C(O)C1NC(C)=O ; *OCNC(=O)CC(=O)NCCCCCCOC1OC(CNC(=O)Cc2cccc(OC)c2)C(O)C(O)C1NC(C)=O ; *OCNC(=O)CC(=O)NCCCCCCOC1OC(CNC(=O)CCc2cccc(OC)c2)C(O)C(O)C1NC(C)=O]

### Example 41: Single Dose Study of Modified Oligonucleotides Targeting SRB1 In Vivo

The compounds in Table 41 were designed to target mouse SRB1. Wild type mice were injected subcutaneously once with a modified oligonucleotide listed in Table 41 at 4.5 mg/kg. A control group was injected subcutaneously with PBS. Each treatment group consisted of 2-4 animals. The mice were sacrificed 72 hours following oligonucleotide administration to determine the liver SRB1 mRNA levels using real-time PCR according to standard protocols. SRB1 mRNA levels were determined relative to total RNA (using RIBOGREEN®), prior to normalization to PBS treated control. The results below are presented as the average percent of SRB1 mRNA levels for each treatment group relative to the average for the PBS treated group. The results below illustrate that the oligonucleotides comprising various modified GalNAc sugars decreased target mRNA levels and several oligonucleotides comprising various modified GalNAc sugars were more potent than the oligonucleotide comprising an unmodified GalNAc sugar (GalNAc₁-37ₐ).

TABLE 41
Activity of modified oligonucleotides targeting mouse SRB1

| | | SRB1 mRNA | SEQ |
|---|---|---|---|
| Isis No. | Sequence | (% PBS) | ID NO. |
| 736690 | GalNAc₁-37_{a-o′}Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ | 29 | 147 |
| | ^{m}Cₑₛ^{m}CₑₛTₑₛTₑ | | |
| 727852 | GalNAc₁-34_{a-o′} Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ | 23 | 147 |
| | ^{m}Cₑₛ^{m}CₑₛTₑₛTₑ | | |
| 790394 | GalNAc₁-52_{a-o′} Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ | 33 | 147 |
| | ^{m}Cₑₛ^{m}CₑₛTₑₛTₑ | | |
| 790437 | GalNAc₁-53_{a-o′} Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ | 29 | 147 |
| | ^{m}Cₑₛ^{m}CₑₛTₑₛTₑ | | |
| 789773 | GalNAc₁-54_{a-o′} Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ | 29 | 147 |
| | ^{m}Cₑₛ^{m}CₑₛTₑₛTₑ | | |
| 789793 | GalNAc₁-55_{a-o′} Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ | 33 | 147 |
| | ^{m}Cₑₛ^{m}CₑₛTₑₛTₑ | | |
| 790393 | GalNAc₁-56_{a-o′} Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ | 26 | 147 |
| | ^{m}Cₑₛ^{m}CₑₛTₑₛTₑ | | |
| 789774 | GalNAc₁-57_{a-o′} Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ | 33 | 147 |
| | ^{m}Cₑₛ^{m}CₑₛTₑₛTₑ | | |
| 790436 | GalNAc₁-58_{a-o′} Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ | 28 | 147 |
| | ^{m}Cₑₛ^{m}CₑₛTₑₛTₑ | | |

The structures of GalNAc₁-34ₐ and GalNAc₁-37ₐ are described above. Compounds comprising GalNAc₁-52ₐ, GalNAc₁-53ₐ, GalNAc₁-54ₐ, GalNAc₁-55ₐ, GalNAc₁-56ₐ, GalNAc₁-57ₐ, and GalNAc₁-58ₐ were made using synthetic routes described herein, routes similar to those described herein, or reactions known in the art. The structures are shown below:

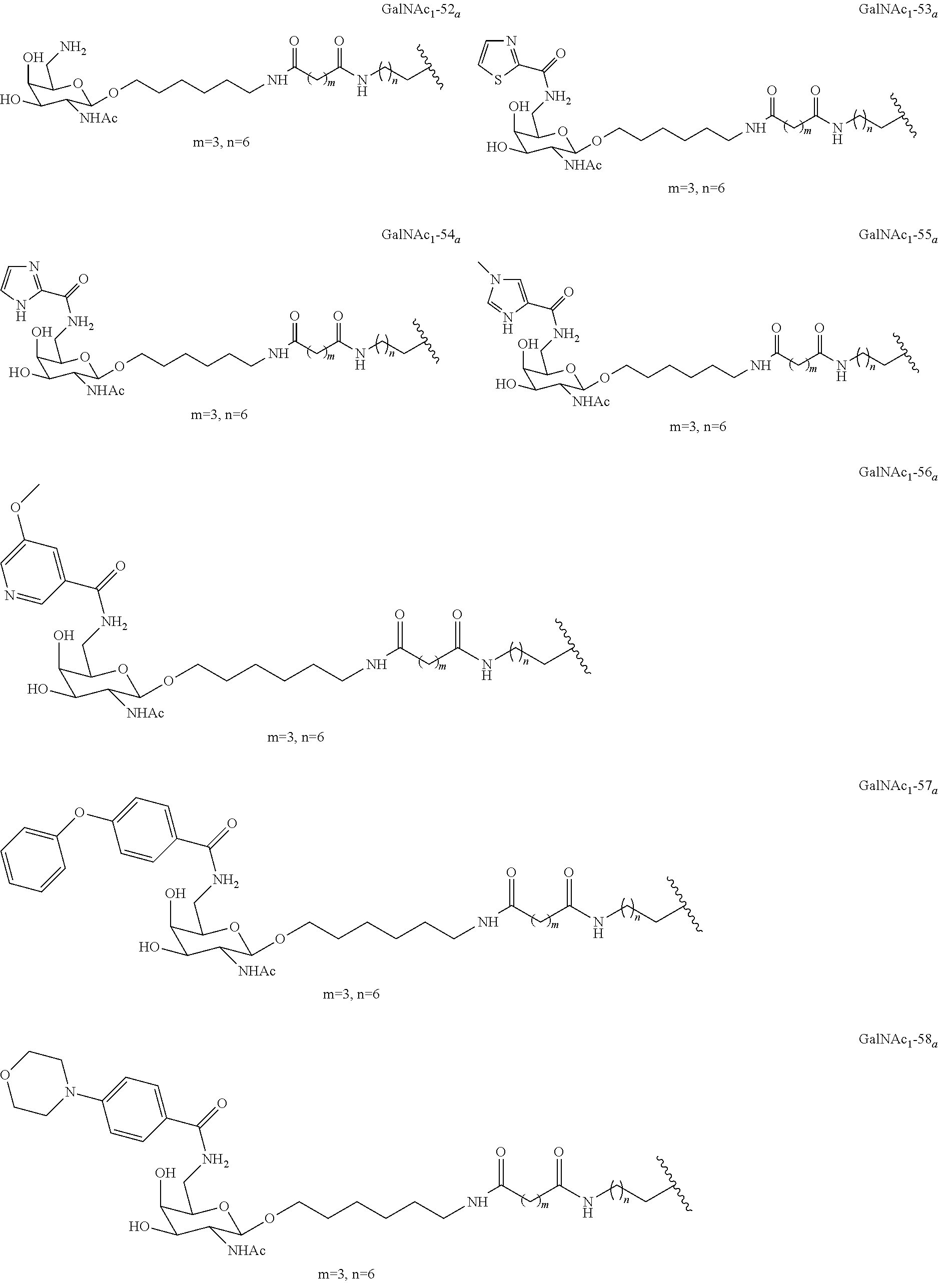

### Example 42: Single Dose Study of Modified Oligonucleotides Targeting SRB1 In Vivo

The compounds in Table 42 were designed to target mouse SRB1. Wild type mice were injected subcutaneously once with a modified oligonucleotide listed in Table 42 at 4.5 mg/kg. A control group was injected subcutaneously with PBS. Each treatment group consisted of 2-4 animals. The mice were sacrificed 72 hours following oligonucleotide administration to determine the liver SRB1 mRNA levels using real-time PCR according to standard protocols. SRB1 mRNA levels were determined relative to total RNA (using RIBOGREEN®), prior to normalization to PBS treated control. The results below are presented as the average percent of SRB1 mRNA levels for each treatment group relative to the average for the PBS treated group. The results below illustrate that the oligonucleotides comprising various modified GalNAc sugars decreased target mRNA levels and several oligonucleotides comprising various modified GalNAc sugars were more potent than the oligonucleotide comprising an unmodified GalNAc sugar (GalNAc₁-37ₐ).

TABLE 42
Activity of modified oligonucleotides targeting mouse SRB1

| | | SRB1 mRNA | SEQ |
|---|---|---|---|
| Isis No. | Sequence | (% PBS) | ID NO. |
| 736690 | GalNAc₁-37_{a-o′}Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ | 41 | 147 |
| | ^{m}Cₑₛ^{m}CₑₛTₑₛTₑ | | |
| 752534 | GalNAc₁-59_{a-o′} Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ | 36 | 147 |
| | ^{m}Cₑₛ^{m}CₑₛTₑₛTₑ | | |
| 752533 | GalNAc₁-60_{a-o′} Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ | 39 | 147 |
| | ^{m}Cₑₛ^{m}CₑₛTₑₛTₑ | | |
| 736694 | GalNAc₁-61_{a-o′} Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ | 45 | 147 |
| | ^{m}Cₑₛ^{m}CₑₛTₑₛTₑ | | |
| 754154 | GalNAc₁-62_{a-o′} Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ | 51 | 147 |
| | ^{m}Cₑₛ^{m}CₑₛTₑₛTₑ | | |

The structure of GalNAc₁-37ₐ is described above. Compounds comprising GalNAc₁-59ₐ, GalNAc₁-60ₐ, GalNAc₁-61ₐ, and GalNAc₁-62ₐ were made using synthetic routes described herein, routes similar to those described herein, or reactions known in the art. The structures are shown below:

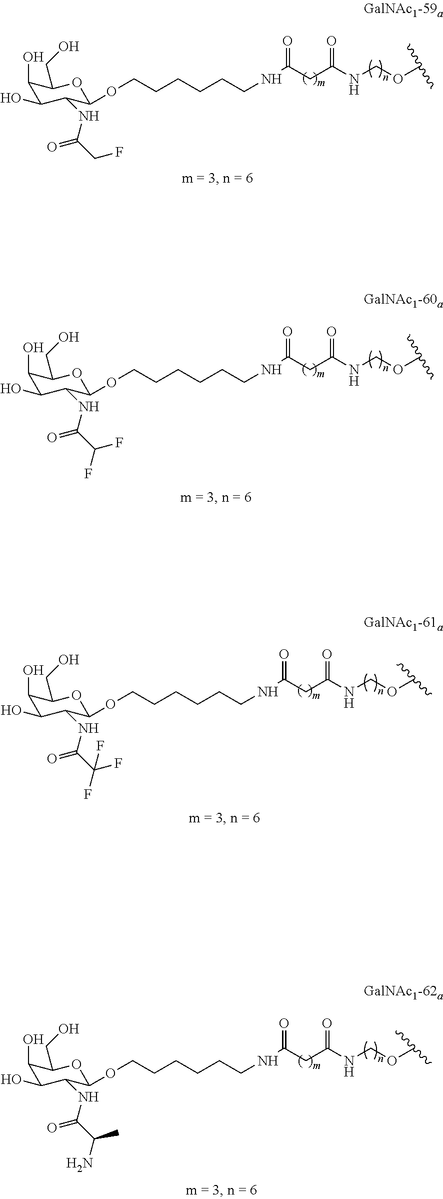

### Example 43: Single Dose Study of Modified Oligonucleotides Targeting SRB1 In Vivo

The compounds in Table 43 were designed to target mouse SRB1. Wild type mice were injected subcutaneously once with a modified oligonucleotide listed in Table 43 at 4.5 mg/kg. A control group was injected subcutaneously with PBS. Each treatment group consisted of 2-4 animals. The mice were sacrificed 72 hours following oligonucleotide administration to determine the liver SRB1 mRNA levels using real-time PCR according to standard protocols. SRB1 mRNA levels were determined relative to total RNA (using RIBOGREEN®), prior to normalization to PBS treated control. The results below are presented as the average percent of SRB1 mRNA levels for each treatment group relative to the average for the PBS treated group. The results below illustrate that the oligonucleotides comprising various modified GalNAc sugars decreased target mRNA levels and several oligonucleotides comprising various modified GalNAc sugars were more potent than the oligonucleotide comprising an unmodified GalNAc sugar (GalNAc₁-37ₐ).

TABLE 43
Activity of modified oligonucleotides targeting mouse SRB1

| | | SRB1 mRNA | SEQ |
|---|---|---|---|
| Isis No. | Sequence | (% PBS) | ID NO. |
| 736690 | GalNAc₁-37_{a-o′}Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ | 41 | 147 |
| | ^{m}Cₑₛ^{m}CₑₛTₑₛTₑ | | |
| 748827 | GalNAc₁-63_{a-o′} Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ | 77 | 147 |
| | ^{m}Cₑₛ^{m}CₑₛTₑₛTₑ | | |
| 739254 | GalNAc₁-64_{a-o′} Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ | 90 | 147 |
| | ^{m}Cₑₛ^{m}CₑₛTₑₛTₑ | | |
| 739233 | GalNAc₁-65_{a-o′} Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ | 78 | 147 |
| | ^{m}Cₑₛ^{m}CₑₛTₑₛTₑ | | |
| 737333 | GalNAc₁-66_{a-o′} Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ | 91 | 147 |
| | ^{m}Cₑₛ^{m}CₑₛTₑₛTₑ | | |
| 736689 | GalNAc₁-67_{a-o′} Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ | 98 | 147 |
| | ^{m}Cₑₛ^{m}CₑₛTₑₛTₑ | | |

The structure of GalNAc₁-37ₐ is described above. Compounds comprising GalNAc₁-63ₐ, GalNAc₁-64ₐ, GalNAc₁-65ₐ, GalNAc₁-66ₐ, and GalNAc₁-67ₐ were made using synthetic routes described herein, routes similar to those described herein, or reactions known in the art. The structures are shown below:

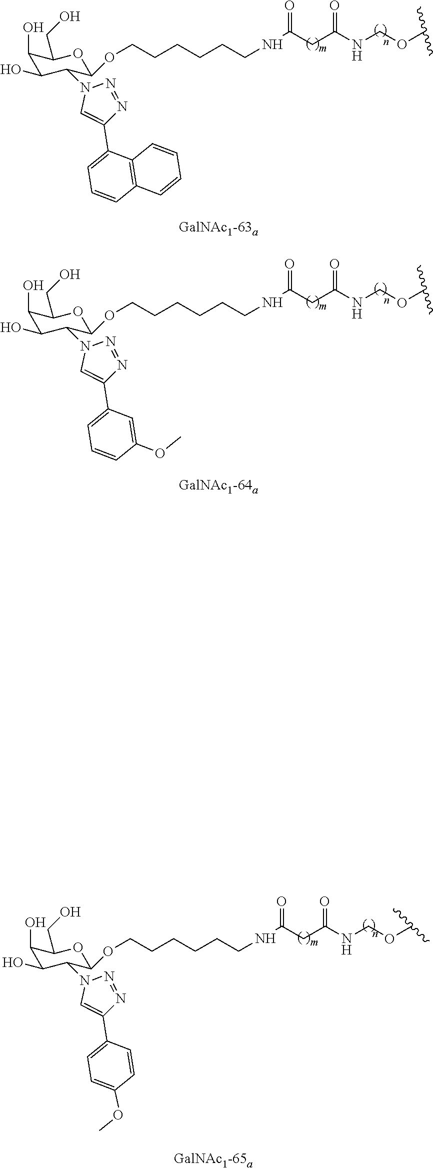 [SMILES: *OCNC(=O)CC(=O)NCCCCCCOC1OC(CO)C(O)C(O)C1n1cc(-c2cccc3ccccc23)nn1 ; *OCNC(=O)CC(=O)NCCCCCCOC1OC(CO)C(O)C(O)C1n1cc(-c2cccc(OC)c2)nn1 ; *OCNC(=O)CC(=O)NCCCCCCOC1OC(CO)C(O)C(O)C1n1cc(-c2ccc(OC)cc2)nn1]

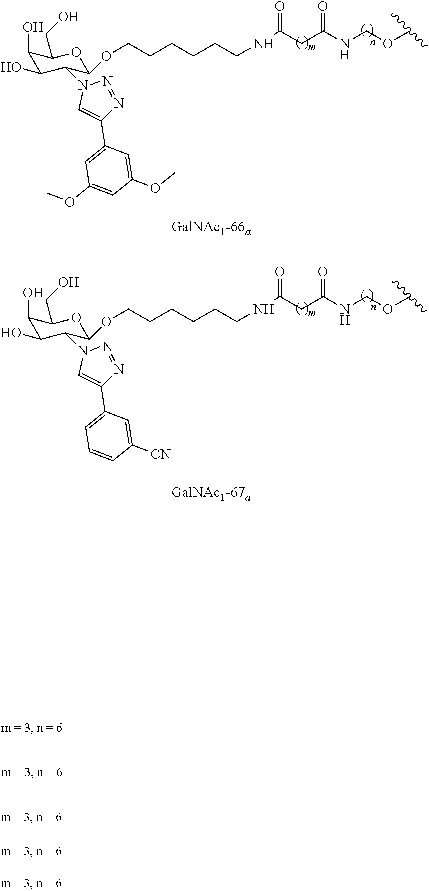 [SMILES: *OCNC(=O)CC(=O)NCCCCCCOC1OC(CO)C(O)C(O)C1n1cc(-c2cc(OC)cc(OC)c2)nn1 ; *OCNC(=O)CC(=O)NCCCCCCOC1OC(CO)C(O)C(O)C1n1cc(-c2cccc(C#N)c2)nn1]

### Example 44: Single Dose Study of Modified Oligonucleotides Targeting SRB1 In Vivo

The compounds in Table 44 were designed to target mouse SRB1. Wild type mice were injected subcutaneously once with a modified oligonucleotide listed in Table 44 at 4.5 mg/kg. A control group was injected subcutaneously with PBS. Each treatment group consisted of 2-4 animals. The mice were sacrificed 72 hours following oligonucleotide administration to determine the liver SRB1 mRNA levels using real-time PCR according to standard protocols. SRB1 mRNA levels were determined relative to total RNA (using RIBOGREEN®), prior to normalization to PBS treated control. The results below are presented as the average percent of SRB1 mRNA levels for each treatment group relative to the average for the PBS treated group. The results below illustrate that the oligonucleotides comprising various modified GalNAc sugars decreased target mRNA levels and several oligonucleotides comprising various modified GalNAc sugars were more potent than the oligonucleotide comprising an unmodified GalNAc sugar (GalNAc₁-37ₐ).

TABLE 44
Activity of modified oligonucleotides targeting mouse SRB1

| | | SRB 1 mRNA | SEQ |
|---|---|---|---|
| Isis No. | Sequence | (% PBS) | ID NO. |
| 736690 | GalNAc₁-37_{a-o′}Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ | 67 | 147 |
| | ^{m}Cₑₛ^{m}CₑₛTₑₛTₑ | | |
| 727852 | GalNAc₁-34_{a-o′} Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ | 53 | 147 |
| | ^{m}Cₑₛ^{m}CₑₛTₑₛTₑ | | |
| 801355 | GalNAc₁-68_{a-o′} Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ | 67 | 147 |
| | ^{m}Cₑₛ^{m}CₑₛTₑₛTₑ | | |
| 801356 | GalNAc₁-69_{a-o′} Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ | 66 | 147 |
| | ^{m}Cₑₛ^{m}CₑₛTₑₛTₑ | | |
| 801373 | GalNAc₁-70_{a-o′} Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ | 50 | 147 |
| | ^{m}Cₑₛ^{m}CₑₛTₑₛTₑ | | |

The structures of GalNAc₁-34ₐ and GalNAc₁-37ₐ are described above. Compounds comprising GalNAc₁-68ₐ, GalNAc₁-69ₐ, and GalNAc₁-70ₐ were made using synthetic routes described herein, routes similar to those described herein, or reactions known in the art. The structures are shown below:

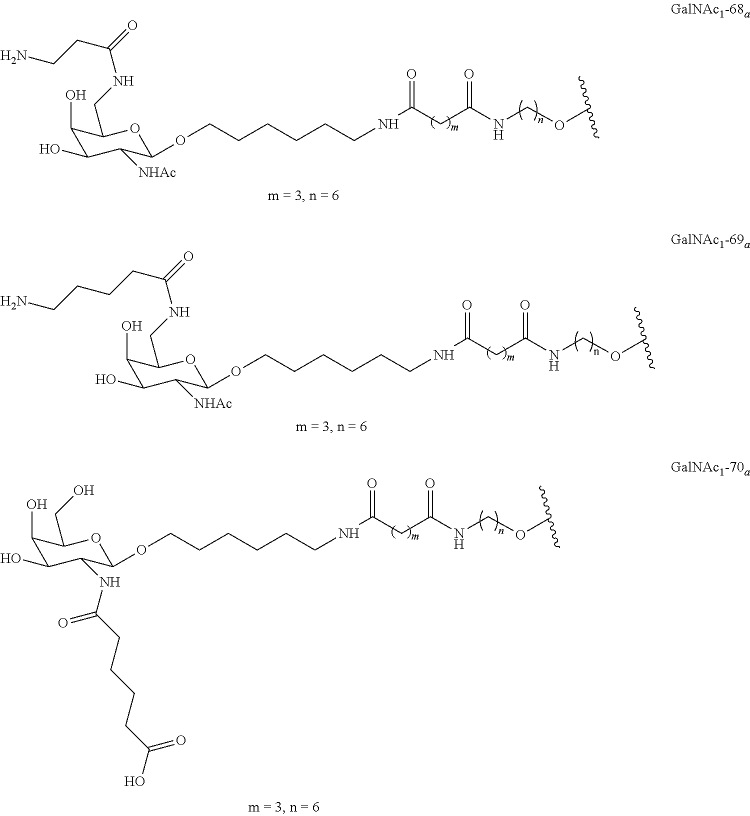

### Example 45: Single Dose Study of Modified Oligonucleotides Targeting SRB1 In Vivo

The compounds in Table 45 were designed to target mouse SRB1. Wild type mice were injected subcutaneously once with a modified oligonucleotide listed in Table 45 at 4.5 mg/kg. A control group was injected subcutaneously with PBS. Each treatment group consisted of 2-4 animals. The mice were sacrificed 72 hours following oligonucleotide administration to determine the liver SRB1 mRNA levels using real-time PCR according to standard protocols. SRB1 mRNA levels were determined relative to total RNA (using RIBOGREEN®), prior to normalization to PBS treated control. The results below are presented as the average percent of SRB1 mRNA levels for each treatment group relative to the average for the PBS treated group. The results below illustrate that the oligonucleotides comprising various modified GalNAc sugars decreased target mRNA levels and several oligonucleotides comprising various modified GalNAc sugars were more potent than the oligonucleotide comprising an unmodified GalNAc sugar (GalNAc₁-37ₐ).

TABLE 45
Activity of modified oligonucleotides targeting mouse SRB1

| | | SRB1 mRNA | SEQ |
|---|---|---|---|
| Isis No. | Sequence | (% PBS) | ID NO. |
| 736690 | GalNAc₁-37_{a-o′}Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ | 41 | 147 |
| | ^{m}Cₑₛ^{m}CₑₛTₑₛTₑ | | |
| 727852 | GalNAc₁-34_{a-o′} Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ | 35 | 147 |
| | ^{m}Cₑₛ^{m}CₑₛTₑₛTₑ | | |
| 752534 | GalNAc₁-59_{a-o′} Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ | 36 | 147 |
| | ^{m}Cₑₛ^{m}CₑₛTₑₛTₑ | | |
| 801374 | GalNAc₁-71_{a-o′} Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ | 54 | 147 |
| | ^{m}Cₑₛ^{m}CₑₛTₑₛTₑ | | |

The structures of GalNAc₁-34ₐ, GalNAc₁-37ₐ, and GalNAc₁-59ₐ are described above. Isis No. 801374, comprising GalNAc₁-71ₐ, was made using synthetic routes described herein, routes similar to those described herein, or reactions known in the art. The structures are shown below:

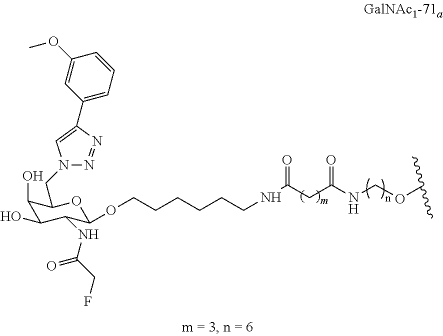

### Example 46: Single Dose Study of Modified Oligonucleotides Targeting SRB1 In Vivo

The compounds in Table 46 were designed to target mouse SRB1. Wild type mice were injected subcutaneously once with a modified oligonucleotide listed in Table 46 at 4.5 mg/kg. A control group was injected subcutaneously with PBS. Each treatment group consisted of 2-4 animals. The mice were sacrificed 72 hours following oligonucleotide administration to determine the liver SRB1 mRNA levels using real-time PCR according to standard protocols. SRB1 mRNA levels were determined relative to total RNA (using RIBOGREEN®), prior to normalization to PBS treated control. The results below are presented as the average percent of SRB1 mRNA levels for each treatment group relative to the average for the PBS treated group. The results below illustrate that the oligonucleotides comprising various modified GalNAc sugars decreased target mRNA levels and were more potent than the oligonucleotide comprising one unmodified GalNAc sugar (GalNAc₁-37ₐ) and no modified GalNAc sugars.

TABLE 46
Activity of modified oligonucleotides targeting mouse SRB1

| | | SRB1 mRNA | SEQ |
|---|---|---|---|
| Isis No. | Sequence | (% PBS) | ID NO. |
| 736690 | GalNAc₁-37_{a-o′}Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ | 55 | 147 |
| | ^{m}Cₑₛ^{m}CₑₛTₑₛTₑ | | |
| 727852 | GalNAc₁-34_{a-o′} Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ | 44 | 147 |
| | ^{m}Cₑₛ^{m}CₑₛTₑₛTₑ | | |
| 765153 | GalNAc2-72_{a-o′} Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ | 49 | 147 |
| | ^{m}Cₑₛ^{m}CₑₛTₑₛTₑ | | |
| 765154 | GalNAc₁-73_{a-o′} Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ | 51 | 147 |
| | ^{m}Cₑₛ^{m}CₑₛTₑₛTₑ | | |

The structures of GalNAc₁-34ₐ and GalNAc₁-37ₐ are described above. Compounds comprising GalNAc₂-72ₐ and GalNAc₁-73ₐ were made using synthetic routes described herein, routes similar to those described herein, or reactions known in the art. The structures are shown below:

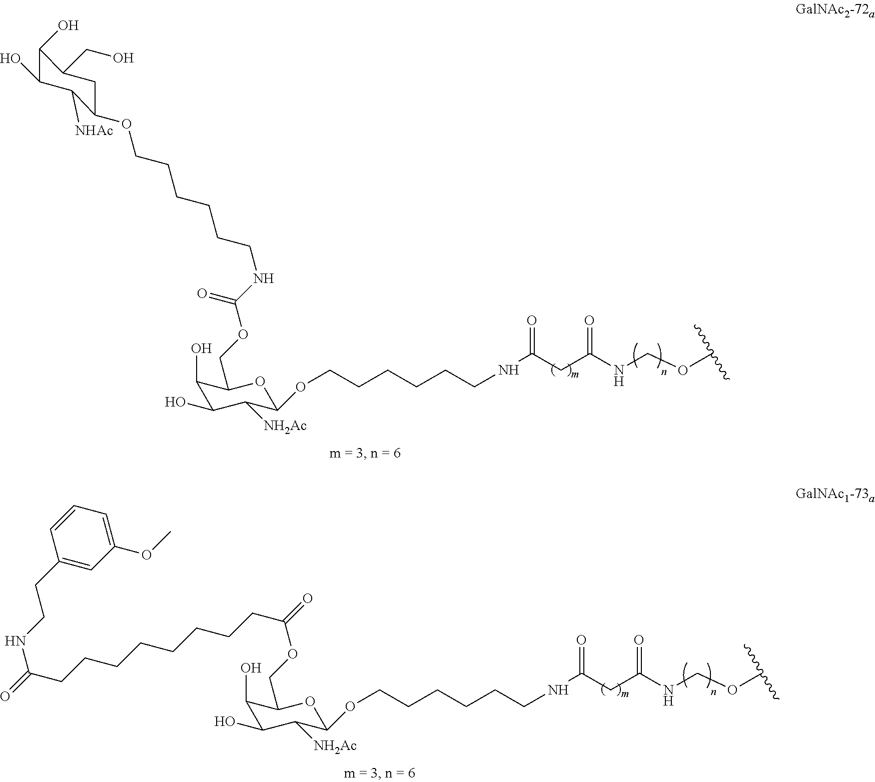

### Example 47: Single Dose Study of Modified Oligonucleotides Targeting SRB1 In Vivo

The compounds in Table 47 were designed to target mouse SRB1. Wild type mice were injected subcutaneously once with a modified oligonucleotide listed in Table 47 at 4.5 mg/kg. A control group was injected subcutaneously with PBS. Each treatment group consisted of 2-4 animals. The mice were sacrificed 72 hours following oligonucleotide administration to determine the liver SRB1 mRNA levels using real-time PCR according to standard protocols. SRB1 mRNA levels were determined relative to total RNA (using RIBOGREEN®), prior to normalization to PBS treated control. The results below are presented as the average percent of SRB1 mRNA levels for each treatment group relative to the average for the PBS treated group. The results below illustrate that the oligonucleotides comprising various modified GalNAc sugars decreased target mRNA levels.

TABLE 47
Activity of modified oligonucleotides targeting mouse SRB1

| | | SRB1 mRNA | SEQ |
|---|---|---|---|
| Isis No. | Sequence | (% PBS) | ID NO. |
| 736690 | GalNAc₁-37_{a-o′}Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ | 41 | 147 |
| | ^{m}Cₑₛ^{m}CₑₛTₑₛTₑ | | |
| 727852 | GalNAc₁-34_{a-o′} Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ | 43 | 147 |
| | ^{m}Cₑₛ^{m}CₑₛTₑₛTₑ | | |
| 761854 | GalNAc₁-51_{a-o′} Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ | 52 | 147 |
| | ^{m}Cₑₛ^{m}CₑₛTₑₛTₑ | | |
| 761855 | GalNAc₁-74_{a-o′} Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ | 51 | 147 |
| | ^{m}Cₑₛ^{m}CₑₛTₑₛTₑ | | |

The structures of GalNAc₁-34ₐ, GalNAc₁-37ₐ, and GalNAc₁-51ₐ are described above. Isis No. 761855 comprising GalNAc₁-74ₐ was made using synthetic routes described herein, routes similar to those described herein, or reactions known in the art. The structures are shown below:

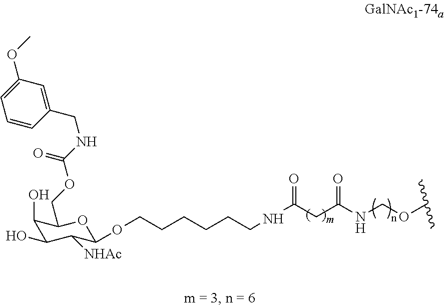

### Example 48: Single Dose Study of Modified Oligonucleotides Targeting SRB1 In Vivo

The compounds in Table 48 were designed to target mouse SRB1. Wild type mice were injected subcutaneously once with a modified oligonucleotide listed in Table 48 at 4.5 mg/kg. A control group was injected subcutaneously with PBS. Each treatment group consisted of 2-4 animals. The mice were sacrificed 72 hours following oligonucleotide administration to determine the liver SRB1 mRNA levels using real-time PCR according to standard protocols. SRB1 mRNA levels were determined relative to total RNA (using RIBOGREEN®), prior to normalization to PBS treated control. The results below are presented as the average percent of SRB1 mRNA levels for each treatment group relative to the average for the PBS treated group. The results below illustrate that the oligonucleotides comprising various modified GalNAc sugars decreased target mRNA levels.

TABLE 48
Activity of modified oligonucleotides targeting mouse SRB1

| | | SRB1 mRNA | SEQ |
|---|---|---|---|
| Isis No. | Sequence | (% PBS) | ID NO. |
| 736690 | GalNAc₁-37_{a-o′}Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ | 41 | 147 |
| | ^{m}Cₑₛ^{m}CₑₛTₑₛTₑ | | |
| 801359 | GalNAc₁-43_{a-o′} Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ | 52 | 147 |
| | ^{m}Cₑₛ^{m}CₑₛTₑₛTₑ | | |
| 752376 | GalNAc₁-75_{a-o′} Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ | 38 | 147 |
| | ^{m}Cₑₛ^{m}CₑₛTₑₛTₑ | | |
| 790394 | GalNAc₁-52_{a-o′} Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ | 33 | 147 |
| | ^{m}Cₑₛ^{m}CₑₛTₑₛTₑ | | |

The structures of GalNAc₁-37ₐ, GalNAc₁-43ₐ, and GalNAc₁-52ₐ are described above. Isis No. 752376 comprising GalNAc₁-75ₐ was made using synthetic routes described herein, routes similar to those described herein, or reactions known in the art. The structures are shown below:

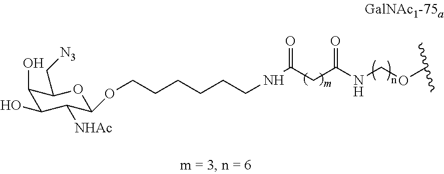

### Example 49: Single Dose Study of Modified Oligonucleotides Targeting SRB1 In Vivo

The compounds in Table 49 were designed to target mouse SRB1. Wild type mice were injected subcutaneously once with a modified oligonucleotide listed in Table 49 at 4.5 mg/kg. A control group was injected subcutaneously with PBS. Each treatment group consisted of 2-4 animals. The mice were sacrificed 72 hours following oligonucleotide administration to determine the liver SRB1 mRNA levels using real-time PCR according to standard protocols. SRB1 mRNA levels were determined relative to total RNA (using RIBOGREEN®), prior to normalization to PBS treated control. The results below are presented as the average percent of SRB1 mRNA levels for each treatment group relative to the average for the PBS treated group. The results below illustrate that the oligonucleotides comprising GalNAc sugars decreased target mRNA levels.

TABLE 49
Activity of modified oligonucleotides targeting mouse SRB1

| | | SRB1 mRNA | SEQ |
|---|---|---|---|
| Isis No. | Sequence | (% PBS) | ID NO. |
| 762827 | GalNAc₁-76_{a-o′} Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ | 39 | 147 |
| | ^{m}Cₑₛ^{m}CₑₛTₑₛTₑₒGalNAc₁-76ₐ | | |
| 773493 | Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ^{m}Cₑₛ^{m}CₑₛTₑₛTₑₒGalNAc₂- | 44 | |
| | 77ₐ | | |

Isis No. 762827 comprising GalNAc₁-76ₐ at both the 5′-terminal nucleoside and GalNAc₁-76ₐ at the 3′-terminal nucleoside was made using synthetic routes described herein, routes similar to those described herein, or reactions known in the art. Isis No. 773493 comprising GalNAc₂-77ₐ was made using synthetic routes described herein, routes similar to those described herein, or reactions known in the art. The structures of GalNAc₁-76ₐ and GalNAc₂-77ₐ are shown below:

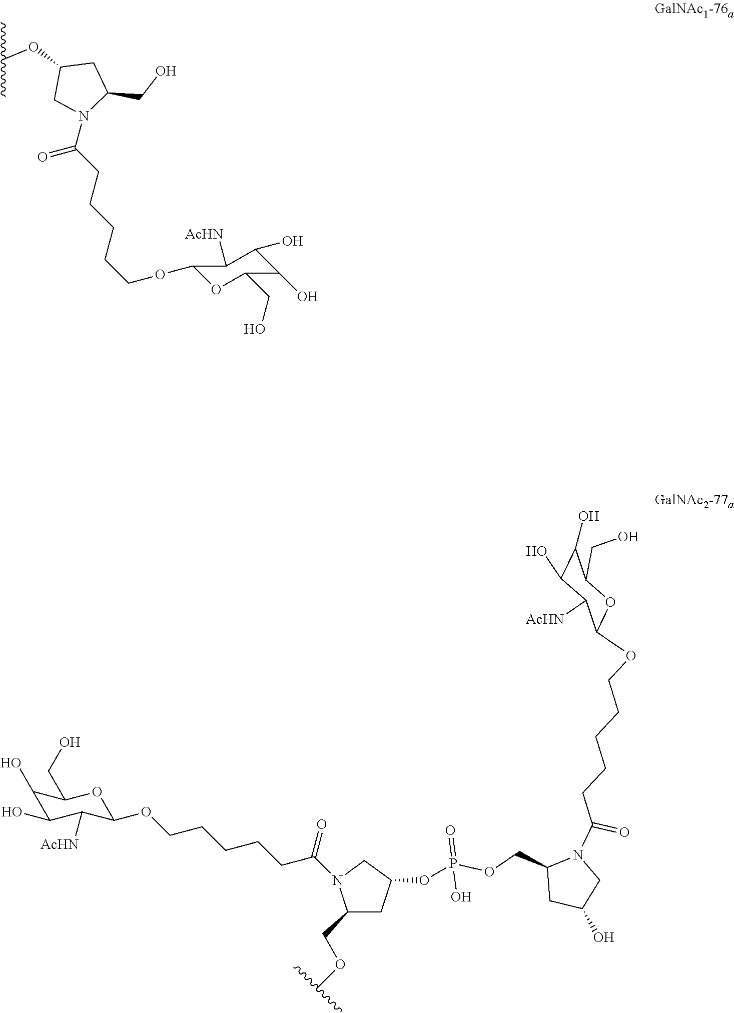 [SMILES: *O[C@@H]1C[C@@H](CO)N(C(=O)CCCCCOC2OC(CO)C(O)C(O)C2NC(C)=O)C1 ; *OC[C@@H]1C[C@@H](OP(=O)(O)OC[C@@H]2C[C@@H](O)CN2C(=O)CCCCCOC2OC(CO)C(O)C(O)C2NC(C)=O)CN1C(=O)CCCCCOC1OC(CO)C(O)C(O)C1NC(C)=O]

### Example 50: Bioavailability of Antisense Oligonucleotides Comprising a GalNAc Cluster Administered Intrajejunally

Antisense oligonucleotides targeting rat MALAT-1, shown in Table 50 below, were tested in a bioavailability study in rat. Isis Numbers 704361 and 748293 are identical except that 704361 comprises a cleavable phosphate moiety between the conjugate group and the rest of the oligonucleotide, and 748293 comprises a stable phosphorothioate moiety between the conjugate group and the rest of the oligonucleotide. Isis Numbers 704361 and 748293 were formulated using a sodium caprate (C10) vehicle. Prior to treatment, rats were fasted overnight, and the jejunums and portal veins were cannulated. A single dose of 20 or 50 mg/kg oligonucleotide was then intrajejunally administered to Sprague Dawley rats. Each treatment group consisted of 4 animals. Fifteen minutes after oligonucleotide administration, a portal vein sample was taken. HPLC-MS analysis of the portal vein samples from rats treated with Isis No. 704361 and 748293 showed that both oligonucleotides comprising a GalNAc₃ conjugate group were greater than 90% intact following absorption. Three days following oligonucleotide administration, the rats were sacrificed and liver levels of the intact oligonucleotides were analyzed by HPLC-MS. The results are shown in Table 51 below as the absolute concentrations of the intact oligonucleotides in the liver.

TABLE 50
Antisense oligonucleotides for use in bioavailability testing in rat

| ISIS | | SEQ |
|---|---|---|
| No. | Sequences (5′ to 3′) | ID No. |
| 556116 | Aₖₛ^{m}Cₖₛ^{m}CₖₛA_{ds}T_{ds}G_{ds}A_{ds}T_{ds}A_{ds}^{m}C_{ds}^{m}C_{ds}A_{ds}^{m}C_{ds}TₖₛTₖₛTₖ | 146 |
| 704361 | GalNAC₃-7_{a-o′} Aₖₛ^{m}Cₖₛ^{m}CₖₛA_{ds}T_{ds}G_{ds}A_{ds}T_{ds}A_{ds}^{m}C_{ds}^{m}C_{ds}A_{ds}^{m}C_{ds}T_{ds}T_{ds}Tₖ | 146 |
| 748293 | GalNAC₃-7_{a-s′} Aₖₛ^{m}Cₖₛ^{m}CₖₛA_{ds}T_{ds}G_{ds}A_{ds}T_{ds}A_{ds}^{m}C_{ds}^{m}C_{ds}A_{ds}^{m}C_{ds}TₖₛTₖₛTₖ | 146 |

Subscript “k” indicates a cEt modified nucleoside, and “s′” indicates —O—P(═S)(OH)—. See Table 23 for list of other abbreviations.

TABLE 51
Liver concentration of antisense oligonucleotides
administered intrajejunally

| | Vehicle | | Oligonucleotide | Liver |
|---|---|---|---|---|
| | Dosage | Isis | Dosage | concentration |
| Vehicle | (mg/kg) | No. | (mg/kg) | (^{μ}M) |
| C10 | 150 | 556116 | 50 | 0.0031 |
| C10 | 150 | 704361 | 20 | 1.0669 |
| C10 | 150 | 704361 | 50 | 1.3406 |
| C10 | 150 | 748293 | 50 | 4.1803 |

### Example 51: Efficacy of Antisense Oligonucleotides Comprising a GalNAc Cluster Administered Intrajejunally

Antisense oligonucleotides targeting rat MALAT-1, shown in Table 50 above, were tested for target knock-down in rat. Prior to treatment, rats were fasted overnight, and jejunums were cannulated. A single dose of 20 or 50 mg/kg of oligonucleotide or saline was then intrajejunally administered to Sprague Dawley rats. Each treatment group consisted of 3 or 4 animals. 48 hours following oligonucleotide administration, the rats were sacrificed. Liver levels of the intact oligonucleotides were analyzed by HPLC-MS, and liver mRNA levels of MALAT1 were analyzed via RT-qPCR and normalized to Ribogreen. The results are shown in Table 52 below as the mass of intact oligonucleotide relative to total mass in the liver and as percent normalized MALAT1 mRNA levels relative to the saline treated control.

TABLE 52
Liver PK and PD of antisense oligonucleotides
administered intrajejunally

| | | | Oligonu- | | |
|---|---|---|---|---|---|
| | Vehicle | | cleotide | Liver con- | |
| | Dosage | Isis | Dosage | centration | MALAT1 mRNA |
| Vehicle | (mg/kg) | No. | (mg/kg) | (^{μ}g/g) | (% control) |
| C10 | 150 | 556116 | 50 | 3.35 | 19.0 |
| C10 | 150 | 704361 | 20 | 3.48 | 25.1 |

1. A composition comprising a single stranded antisense oligomeric compound for non-parenteral administration comprising:
a 5′-region consisting of 2-5 linked 5′-region nucleosides;
a 3′-region consisting of 2-5 linked 3′-region nucleosides;
a central region located between the 5′-region and the 3′-region consisting of 10 linked central region deoxynucleosides; and
a conjugate group comprising 3 moieties having the formula: 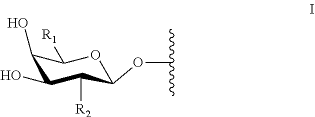
wherein each 5′ and 3′-region nucleoside is a modified nucleoside and each central region nucleoside is a deoxynucleoside;
each R₁ is selected from Q₁, CH₂Q₁, CH₂NJ₁J₂, CH₂N₃ and CH₂SJ₃;
each Q₁ is selected from aryl, substituted aryl, heterocyclic, substituted heterocyclic, heteroaryl and substituted heteroaryl;
each R₂ is selected from N₃, CN, halogen, N(H)C(═O)-Q₂, substituted thiol, aryl, substituted aryl, heterocyclic, substituted heterocyclic, heteroaryl and substituted heteroaryl;
each Q₂ is selected from H, C₁-C₆ alkyl, substituted C₁-C₆ alkyl, C₁-C₆ alkoxy, substituted C₁-C₆ alkoxy, aryl, substituted aryl, heterocyclic, substituted heterocyclic, heteroaryl and substituted heteroaryl;
J₁, J₂ and J₃ are each, independently, H or a substituent group; and
each substituent group is, independently, mono or poly substituted with optionally protected substituent groups independently selected from halogen, C₁-C₆ alkyl, C₁-C₆ alkoxy, aryl, heterocyclic and heteroaryl wherein each substituent group can include a linear or branched alkylene group optionally including one or more groups independently selected from O, S, NH and C(═O), and wherein each substituent group may be further substituted with one or more groups independently selected from C₁-C₆ alkyl, halogen or C₁-C₆ alkoxy wherein each cyclic group is mono or polycyclic; and
an excipient comprising sodium caprate (C10);
wherein said oligomeric compound is at least 95% complementary to a target nucleic acid.

2. The composition of claim 1, wherein the 3 moieties of said formula are linked to the oligomeric compound through a connecting group that comprises a branching group.

3. The composition of claim 1, wherein each R₂ is N(H)C(═O)—CH₃.

4. The composition of claim 1, wherein each R₁ is CH₂Q₁.

5. The composition of claim 4, wherein each Q₁ has the formula: 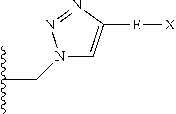
wherein:
E is a single bond or one of said linear or branched alkylene groups; and
X is H or one of said substituent groups.

6. The composition of claim 5, wherein each X is selected from substituted aryl and substituted heteroaryl.

7. The composition of claim 6, wherein each X is phenyl or substituted phenyl comprising one or more substituent groups selected from F, Cl, Br, CO₂Et, OCH₃, CN, CH₃, OCH₃, CF₃, N(CH₃)₂ and O-phenyl.

8. The composition of claim 5, wherein -E-X is selected from among:

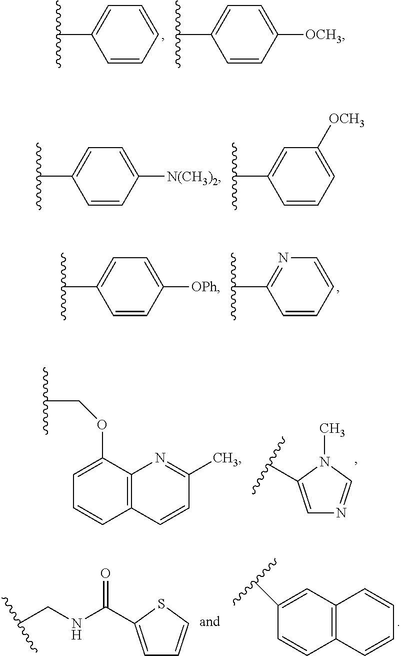

9. The composition of claim 5, wherein -E-X is selected from among:

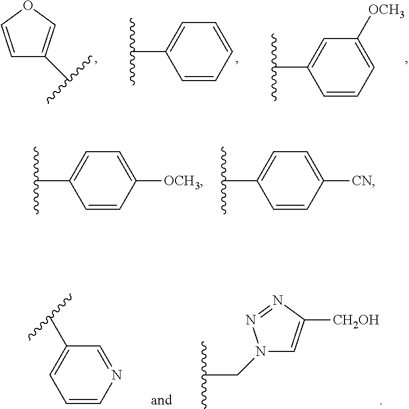

10. The composition of claim 1, wherein each modified nucleoside is independently a bicyclic nucleoside or a 2′-modified nucleoside.

11. The composition of claim 10, wherein each modified nucleoside is a bicyclic nucleoside selected from a 4′-C(CH₃)H—O-2′ or 4′-CH₂—O-2′ bridged bicyclic nucleoside.

12. The composition of claim 10, wherein each modified nucleoside is a 4′-CH(CH₃)—O-2′ bridged bicyclic nucleoside.

13. The composition of claim 10, wherein each modified nucleoside is a 2′-F, 2′-OCH₃ or 2′-O(CH₂)₂OCH₃ substituted nucleoside.

14. The composition of claim 10, wherein each modified nucleoside is a 2′-O(CH₂)₂OCH₃ substituted nucleoside.

15. The composition of claim 1, wherein the conjugate group is attached to the 5′-terminal nucleoside or the 3′-terminal nucleoside of the oligomeric compound.

16. The composition of claim 1, wherein the conjugate group is attached to the 5′-terminal nucleoside of the oligomeric compound.

17. The composition of claim 1, comprising 2 5′-region and 2 3′-region nucleosides.

18. The composition of claim 1, comprising 3 5′-region and 3 3′-region nucleosides.

19. The composition of claim 1, comprising 5 5′-region and 5 3′-region nucleosides.

20. The composition of claim 1, wherein each internucleoside linkage is independently a phosphodiester or a phosphorothioate internucleoside linkage.

21. The composition of claim 1, wherein each internucleoside linkage is a phosphorothioate internucleoside linkage.

22. The composition of claim 1 wherein the oligomeric compound is formulated for said non-parenteral administration as a capsule, tablet, compression coated tablet or bilayer tablet optionally including an enteric coating.

23. The composition of claim 1, wherein the target nucleic acid is an mRNA.

24. The composition of claim 1, wherein the administration is oral.


SEQUENCE LISTING (149 sequences)

SEQ ID NO: 1 - DNA, 10 bp, Homo sapiens
  source 1..10: mol_type = genomic DNA
aaaaaaaaaa

SEQ ID NO: 2 - RNA, 6489 nt, Homo sapiens
  source 1..6489: mol_type = mRNA
aggtacctttggggctggctttctcaaggaagcccagctccctgtgattgagaatgaagtgtgcaatcgctatgactgggattgggacacactttctgggcactgctggccagtcccaaaatggaacataaggaagtggttcttctacttcttttatttctgaaatcagcagcacctgagcaaagccatgtggtccaggattgctaccatggtgatggacagagttatcgaggcacgtactccaccactgtcacaggaaggacctgccaagcttggtcatctatgacaccacatcaacataataggaccacagaaaactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcagaggcctggggtgcaggagtgctaccacggtaatggacagagttatcgaggcacatactccaccactgtcactggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcagaggcctggggtgcaggagtgctaccacggtaatggacagagttatcgaggcacatactccaccactgtcactggaagaacctgccaagcttggtcatctatgacaccacactcgcatagtcggaccccagaatactacccaaatgctggcttgatcatgaactactgcaggaatccagatcctgtggcagccccttattgttatacgagggatcccagtgtcaggtgggagtactgcaacctgacacaatgctcagacgcagaagggactgccgtcgcgcctccaactattaccccgattccaagcctagaggctccttctgaacaagcaccaactgagcaaaggcctggggtgcaggagtgctaccacggaaatggacagagttatcaaggcacatacttcattactgtcacaggaagaacctgccaagcttggtcatctatgacaccacactcgcatagtcggaccccagcatactacccaaatgctggcttgatcaagaactactgccgaaatccagatcctgtggcagccccttggtgttatacaacagatcccagtgtcaggtgggagtactgcaacctgacacgatgctcagatgcagaatggactgccttcgtccctccgaatgttattctggctccaagcctagaggctttttttgaacaagcactgactgaggaaacccccggggtacaggactgctactaccattatggacagagttaccgaggcacatactccaccactgtcacaggaagaacttgccaagcttggtcatctatgacaccacaccagcatagtcggaccccagaaaactacccaaatgctggcctgaccaggaactactgcaggaatccagatgctgagattcgcccttggtgttacaccatggatcccagtgtcaggtgggagtactgcaacctgacacaatgcctggtgacagaatcaagtgtccttgcaactctcacggtggtcccagatccaagcacagaggcttcttctgaagaagcaccaacggagcaaagccccggggtccaggattgctaccatggtgatggacagagttatcgaggctcattctctaccactgtcacaggaaggacatgtcagtcttggtcctctatgacaccacactggcatcagaggacaacagaatattatccaaatggtggcctgaccaggaactactgcaggaatccagatgctgagattagtccttggtgttataccatggatcccaatgtcagatgggagtactgcaacctgacacaatgtccagtgacagaatcaagtgtccttgcgacgtccacggctgtttctgaacaagcaccaacggagcaaagccccacagtccaggactgctaccatggtgatggacagagttatcgaggctcattctccaccactgttacaggaaggacatgtcagtcttggtcctctatgacaccacactggcatcagagaaccacagaatactacccaaatggtggcctgaccaggaactactgcaggaatccagatgctgagattcgcccttggtgttataccatggatcccagtgtcagatgggagtactgcaacctgacgcaatgtccagtgatggaatcaactctcctcacaactcccacggtggtcccagttccaagcacagagcttccttctgaagaagcaccaactgaaaacagcactggggtccaggactgctaccgaggtgatggacagagttatcgaggcacactctccaccactatcacaggaagaacatgtcagtcttggtcgtctatgacaccacattggcatcggaggatcccattatactatccaaatgctggcctgaccaggaactactgcaggaatccagatgctgagattcgcccttggtgttacaccatggatcccagtgtcaggtgggagtactgcaacctgacacgatgtccagtgacagaatcgagtgtcctcacaactcccacagtggccccggttccaagcacagaggctccttctgaacaagcaccacctgagaaaagccctgtggtccaggattgctaccatggtgatggacggagttatcgaggcatatcctccaccactgtcacaggaaggacctgtcaatcttggtcatctatgataccacactggcatcagaggaccccagaaaactacccaaatgctggcctgaccgagaactactgcaggaatccagattctgggaaacaaccctggtgttacacaaccgatccgtgtgtgaggtgggagtactgcaatctgacacaatgctcagaaacagaatcaggtgtcctagagactcccactgttgttccagttccaagcatggaggctcattctgaagcagcaccaactgagcaaacccctgtggtccggcagtgctaccatggtaatggccagagttatcgaggcacattctccaccactgtcacaggaaggacatgtcaatcttggtcatccatgacaccacaccggcatcagaggaccccagaaaactacccaaatgatggcctgacaatgaactactgcaggaatccagatgccgatacaggcccttggtgttttaccatggaccccagcatcaggtgggagtactgcaacctgacgcgatgctcagacacagaagggactgtggtcgctcctccgactgtcatccaggttccaagcctagggcctccttctgaacaagactgtatgtttgggaatgggaaaggataccggggcaagaaggcaaccactgttactgggacgccatgccaggaatgggctgcccaggagccccatagacacagcacgttcattccagggacaaataaatgggcaggtctggaaaaaaattactgccgtaaccctgatggtgacatcaatggtccctggtgctacacaatgaatccaagaaaactttttgactactgtgatatccctctctgtgcatcctcttcatttgattgtgggaagcctcaagtggagccgaagaaatgtcctggaagcattgtaggggggtgtgtggcccacccacattcctggccctggcaagtcagtctcagaacaaggtttggaaagcacttctgtggaggcaccttaatatccccagagtgggtgctgactgctgctcactgcttgaagaagtcctcaaggccttcatcctacaaggtcatcctgggtgcacaccaagaagtgaacctcgaatctcatgttcaggaaatagaagtgtctaggctgttcttggagcccacacaagcagatattgccttgctaaagctaagcaggcctgccgtcatcactgacaaagtaatgccagcttgtctgccatccccagactacatggtcaccgccaggactgaatgttacatcactggctggggagaaacccaaggtacctttgggactggccttctcaaggaagcccagctccttgttattgagaatgaagtgtgcaatcactataagtatatttgtgctgagcatttggccagaggcactgacagttgccagggtgacagtggagggcctctggtttgcttcgagaaggacaaatacattttacaaggagtcacttcttggggtcttggctgtgcacgccccaataagcctggtgtctatgctcgtgtttcaaggtttgttacttggattgagggaatgatgagaaataattaattggacgggagacagagtgaagcatcaacctacttagaagctgaaacgtgggtaaggatttagcatgctggaaataatagacagcaatcaaacgaagacactgttcccagctaccagctatgccaaaccttggcatttttggtatttttgtgtataagcttttaaggtctgactgacaaattctgtattaaggtgtcatagctatgacatttgttaaaaataaactctgcacttattttgatttga

SEQ ID NO: 3 - RNA, 14121 nt, Homo sapiens
  source 1..14121: mol_type = mRNA
attcccaccgggacctgcggggctgagtgcccttctcggttgctgccgctgaggagcccgcccagccagccagggccgcgaggccgaggccaggccgcagcccaggagccgccccaccgcagctggcgatggacccgccgaggcccgcgctgctggcgctgctggcgctgcctgcgctgctgctgctgctgctggcgggcgccagggccgaagaggaaatgctggaaaatgtcagcctggtctgtccaaaagatgcgacccgattcaagcacctccggaagtacacatacaactatgaggctgagagttccagtggagtccctgggactgctgattcaagaagtgccaccaggatcaactgcaaggttgagctggaggttccccagctctgcagcttcatcctgaagaccagccagtgcaccctgaaagaggtgtatggcttcaaccctgagggcaaagccttgctgaagaaaaccaagaactctgaggagtttgctgcagccatgtccaggtatgagctcaagctggccattccagaagggaagcaggttttcctttacccggagaaagatgaacctacttacatcctgaacatcaagaggggcatcatttctgccctcctggttcccccagagacagaagaagccaagcaagtgttgtttctggataccgtgtatggaaactgctccactcactttaccgtcaagacgaggaagggcaatgtggcaacagaaatatccactgaaagagacctggggcagtgtgatcgcttcaagcccatccgcacaggcatcagcccacttgctctcatcaaaggcatgacccgccccttgtcaactctgatcagcagcagccagtcctgtcagtacacactggacgctaagaggaagcatgtggcagaagccatctgcaaggagcaacacctcttcctgcctttctcctacaacaataagtatgggatggtagcacaagtgacacagactttgaaacttgaagacacaccaaagatcaacagccgcttctttggtgaaggtactaagaagatgggcctcgcatttgagagcaccaaatccacatcacctccaaagcaggccgaagctgttttgaagactctccaggaactgaaaaaactaaccatctctgagcaaaatatccagagagctaatctcttcaataagctggttactgagctgagaggcctcagtgatgaagcagtcacatctctcttgccacagctgattgaggtgtccagccccatcactttacaagccttggttcagtgtggacagcctcagtgctccactcacatcctccagtggctgaaacgtgtgcatgccaacccccttctgatagatgtggtcacctacctggtggccctgatccccgagccctcagcacagcagctgcgagagatcttcaacatggcgagggatcagcgcagccgagccaccttgtatgcgctgagccacgcggtcaacaactatcataagacaaaccctacagggacccaggagctgctggacattgctaattacctgatggaacagattcaagatgactgcactggggatgaagattacacctatttgattctgcgggtcattggaaatatgggccaaaccatggagcagttaactccagaactcaagtcttcaatcctcaaatgtgtccaaagtacaaagccatcactgatgatccagaaagctgccatccaggctctgcggaaaatggagcctaaagacaaggaccaggaggttcttcttcagactttccttgatgatgcttctccgggagataagcgactggctgcctatcttatgttgatgaggagtccttcacaggcagatattaacaaaattgtccaaattctaccatgggaacagaatgagcaagtgaagaactttgtggcttcccatattgccaatatcttgaactcagaagaattggatatccaagatctgaaaaagttagtgaaagaagctctgaaagaatctcaacttccaactgtcatggacttcagaaaattctctcggaactatcaactctacaaatctgtttctcttccatcacttgacccagcctcagccaaaatagaagggaatcttatatttgatccaaataactaccttcctaaagaaagcatgctgaaaactaccctcactgcctttggatttgcttcagctgacctcatcgagattggcttggaaggaaaaggctttgagccaacattggaagctctttttgggaagcaaggatttttcccagacagtgtcaacaaagctttgtactgggttaatggtcaagttcctgatggtgtctctaaggtcttagtggaccactttggctataccaaagatgataaacatgagcaggatatggtaaatggaataatgctcagtgttgagaagctgattaaagatttgaaatccaaagaagtcccggaagccagagcctacctccgcatcttgggagaggagcttggttttgccagtctccatgacctccagctcctgggaaagctgcttctgatgggtgcccgcactctgcaggggatcccccagatgattggagaggtcatcaggaagggctcaaagaatgacttttttcttcactacatcttcatggagaatgcctttgaactccccactggagctggattacagttgcaaatatcttcatctggagtcattgctcccggagccaaggctggagtaaaactggaagtagccaacatgcaggctgaactggtggcaaaaccctccgtgtctgtggagtttgtgacaaatatgggcatcatcattccggacttcgctaggagtggggtccagatgaacaccaacttcttccacgagtcgggtctggaggctcatgttgccctaaaagctgggaagctgaagtttatcattccttccccaaagagaccagtcaagctgctcagtggaggcaacacattacatttggtctctaccaccaaaacggaggtgatcccacctctcattgagaacaggcagtcctggtcagtttgcaagcaagtctttcctggcctgaattactgcacctcaggcgcttactccaacgccagctccacagactccgcctcctactatccgctgaccggggacaccagattagagctggaactgaggcctacaggagagattgagcagtattctgtcagcgcaacctatgagctccagagagaggacagagccttggtggataccctgaagtttgtaactcaagcagaaggtgcgaagcagactgaggctaccatgacattcaaatataatcggcagagtatgaccttgtccagtgaagtccaaattccggattttgatgttgacctcggaacaatcctcagagttaatgatgaatctactgagggcaaaacgtcttacagactcaccctggacattcagaacaagaaaattactgaggtcgccctcatgggccacctaagttgtgacacaaaggaagaaagaaaaatcaagggtgttatttccataccccgtttgcaagcagaagccagaagtgagatcctcgcccactggtcgcctgccaaactgcttctccaaatggactcatctgctacagcttatggctccacagtttccaagagggtggcatggcattatgatgaagagaagattgaatttgaatggaacacaggcaccaatgtagataccaaaaaaatgacttccaatttccctgtggatctctccgattatcctaagagcttgcatatgtatgctaatagactcctggatcacagagtccctgaaacagacatgactttccggcacgtgggttccaaattaatagttgcaatgagctcatggcttcagaaggcatctgggagtcttccttatacccagactttgcaagaccacctcaatagcctgaaggagttcaacctccagaacatgggattgccagacttccacatcccagaaaacctcttcttaaaaagcgatggccgggtcaaatataccttgaacaagaacagtttgaaaattgagattcctttgccttttggtggcaaatcctccagagatctaaagatgttagagactgttaggacaccagccctccacttcaagtctgtgggattccatctgccatctcgagagttccaagtccctacttttaccattcccaagttgtatcaactgcaagtgcctctcctgggtgttctagacctctccacgaatgtctacagcaacttgtacaactggtccgcctcctacagtggtggcaacaccagcacagaccatttcagccttcgggctcgttaccacatgaaggctgactctgtggttgacctgctttcctacaatgtgcaaggatctggagaaacaacatatgaccacaagaatacgttcacactatcatgtgatgggtctctacgccacaaatttctagattcgaatatcaaattcagtcatgtagaaaaacttggaaacaacccagtctcaaaaggtttactaatattcgatgcatctagttcctggggaccacagatgtctgcttcagttcatttggactccaaaaagaaacagcatttgtttgtcaaagaagtcaagattgatgggcagttcagagtctcttcgttctatgctaaaggcacatatggcctgtcttgtcagagggatcctaacactggccggctcaatggagagtccaacctgaggtttaactcctcctacctccaaggcaccaaccagataacaggaagatatgaagatggaaccctctccctcacctccacctctgatctgcaaagtggcatcattaaaaatactgcttccctaaagtatgagaactacgagctgactttaaaatctgacaccaatgggaagtataagaactttgccacttctaacaagatggatatgaccttctctaagcaaaatgcactgctgcgttctgaatatcaggctgattacgagtcattgaggttcttcagcctgctttctggatcactaaattcccatggtcttgagttaaatgctgacatcttaggcactgacaaaattaatagtggtgctcacaaggcgacactaaggattggccaagatggaatatctaccagtgcaacgaccaacttgaagtgtagtctcctggtgctggagaatgagctgaatgcagagcttggcctctctggggcatctatgaaattaacaacaaatggccgcttcagggaacacaatgcaaaattcagtctggatgggaaagccgccctcacagagctatcactgggaagtgcttatcaggccatgattctgggtgtcgacagcaaaaacattttcaacttcaaggtcagtcaagaaggacttaagctctcaaatgacatgatgggctcatatgctgaaatgaaatttgaccacacaaacagtctgaacattgcaggcttatcactggacttctcttcaaaacttgacaacatttacagctctgacaagttttataagcaaactgttaatttacagctacagccctattctctggtaactactttaaacagtgacctgaaatacaatgctctggatctcaccaacaatgggaaactacggctagaacccctgaagctgcatgtggctggtaacctaaaaggagcctaccaaaataatgaaataaaacacatctatgccatctcttctgctgccttatcagcaagctataaagcagacactgttgctaaggttcagggtgtggagtttagccatcggctcaacacagacatcgctgggctggcttcagccattgacatgagcacaaactataattcagactcactgcatttcagcaatgtcttccgttctgtaatggccccgtttaccatgaccatcgatgcacatacaaatggcaatgggaaactcgctctctggggagaacatactgggcagctgtatagcaaattcctgttgaaagcagaacctctggcatttactttctctcatgattacaaaggctccacaagtcatcatctcgtgtctaggaaaagcatcagtgcagctcttgaacacaaagtcagtgccctgcttactccagctgagcagacaggcacctggaaactcaagacccaatttaacaacaatgaatacagccaggacttggatgcttacaacactaaagataaaattggcgtggagcttactggacgaactctggctgacctaactctactagactccccaattaaagtgccacttttactcagtgagcccatcaatatcattgatgctttagagatgagagatgccgttgagaagccccaagaatttacaattgttgcttttgtaaagtatgataaaaaccaagatgttcactccattaacctcccattttttgagaccttgcaagaatattttgagaggaatcgacaaaccattatagttgtagtggaaaacgtacagagaaacctgaagcacatcaatattgatcaatttgtaagaaaatacagagcagccctgggaaaactcccacagcaagctaatgattatctgaattcattcaattgggagagacaagtttcacatgccaaggagaaactgactgctctcacaaaaaagtatagaattacagaaaatgatatacaaattgcattagatgatgccaaaatcaactttaatgaaaaactatctcaactgcagacatatatgatacaatttgatcagtatattaaagatagttatgatttacatgatttgaaaatagctattgctaatattattgatgaaatcattgaaaaattaaaaagtcttgatgagcactatcatatccgtgtaaatttagtaaaaacaatccatgatctacatttgtttattgaaaatattgattttaacaaaagtggaagtagtactgcatcctggattcaaaatgtggatactaagtaccaaatcagaatccagatacaagaaaaactgcagcagcttaagagacacatacagaatatagacatccagcacctagctggaaagttaaaacaacacattgaggctattgatgttagagtgcttttagatcaattgggaactacaatttcatttgaaagaataaatgatgttcttgagcatgtcaaacactttgttataaatcttattggggattttgaagtagctgagaaaatcaatgccttcagagccaaagtccatgagttaatcgagaggtatgaagtagaccaacaaatccaggttttaatggataaattagtagagttgacccaccaatacaagttgaaggagactattcagaagctaagcaatgtcctacaacaagttaagataaaagattactttgagaaattggttggatttattgatgatgctgtgaagaagcttaatgaattatcttttaaaacattcattgaagatgttaacaaattccttgacatgttgataaagaaattaaagtcatttgattaccaccagtttgtagatgaaaccaatgacaaaatccgtgaggtgactcagagactcaatggtgaaattcaggctctggaactaccacaaaaagctgaagcattaaaactgtttttagaggaaaccaaggccacagttgcagtgtatctggaaagcctacaggacaccaaaataaccttaatcatcaattggttacaggaggctttaagttcagcatctttggctcacatgaaggccaaattccgagagactctagaagatacacgagaccgaatgtatcaaatggacattcagcaggaacttcaacgatacctgtctctggtaggccaggtttatagcacacttgtcacctacatttctgattggtggactcttgctgctaagaaccttactgactttgcagagcaatattctatccaagattgggctaaacgtatgaaagcattggtagagcaagggttcactgttcctgaaatcaagaccatccttgggaccatgcctgcctttgaagtcagtcttcaggctcttcagaaagctaccttccagacacctgattttatagtccccctaacagatttgaggattccatcagttcagataaacttcaaagacttaaaaaatataaaaatcccatccaggttttccacaccagaatttaccatccttaacaccttccacattccttcctttacaattgactttgtcgaaatgaaagtaaagatcatcagaaccattgaccagatgcagaacagtgagctgcagtggcccgttccagatatatatctcagggatctgaaggtggaggacattcctctagcgagaatcaccctgccagacttccgtttaccagaaatcgcaattccagaattcataatcccaactctcaaccttaatgattttcaagttcctgaccttcacataccagaattccagcttccccacatctcacacacaattgaagtacctacttttggcaagctatacagtattctgaaaatccaatctcctcttttcacattagatgcaaatgctgacatagggaatggaaccacctcagcaaacgaagcaggtatcgcagcttccatcactgccaaaggagagtccaaattagaagttctcaattttgattttcaagcaaatgcacaactctcaaaccctaagattaatccgctggctctgaaggagtcagtgaagttctccagcaagtacctgagaacggagcatgggagtgaaatgctgttttttggaaatgctattgagggaaaatcaaacacagtggcaagtttacacacagaaaaaaatacactggagcttagtaatggagtgattgtcaagataaacaatcagcttaccctggatagcaacactaaatacttccacaaattgaacatccccaaactggacttctctagtcaggctgacctgcgcaacgagatcaagacactgttgaaagctggccacatagcatggacttcttctggaaaagggtcatggaaatgggcctgccccagattctcagatgagggaacacatgaatcacaaattagtttcaccatagaaggacccctcacttcctttggactgtccaataagatcaatagcaaacacctaagagtaaaccaaaacttggtttatgaatctggctccctcaacttttctaaacttgaaattcaatcacaagtcgattcccagcatgtgggccacagtgttctaactgctaaaggcatggcactgtttggagaagggaaggcagagtttactgggaggcatgatgctcatttaaatggaaaggttattggaactttgaaaaattctcttttcttttcagcccagccatttgagatcacggcatccacaaacaatgaagggaatttgaaagttcgttttccattaaggttaacagggaagatagacttcctgaataactatgcactgtttctgagtcccagtgcccagcaagcaagttggcaagtaagtgctaggttcaatcagtataagtacaaccaaaatttctctgctggaaacaacgagaacattatggaggcccatgtaggaataaatggagaagcaaatctggatttcttaaacattcctttaacaattcctgaaatgcgtctaccttacacaataatcacaactcctccactgaaagatttctctctatgggaaaaaacaggcttgaaggaattcttgaaaacgacaaagcaatcatttgatttaagtgtaaaagctcagtataagaaaaacaaacacaggcattccatcacaaatcctttggctgtgctttgtgagtttatcagtcagagcatcaaatcctttgacaggcattttgaaaaaaacagaaacaatgcattagattttgtcaccaaatcctataatgaaacaaaaattaagtttgataagtacaaagctgaaaaatctcacgacgagctccccaggacctttcaaattcctggatacactgttccagttgtcaatgttgaagtgtctccattcaccatagagatgtcggcattcggctatgtgttcccaaaagcagtcagcatgcctagtttctccatcctaggttctgacgtccgtgtgccttcatacacattaatcctgccatcattagagctgccagtccttcatgtccctagaaatctcaagctttctcttccacatttcaaggaattgtgtaccataagccatatttttattcctgccatgggcaatattacctatgatttctcctttaaatcaagtgtcatcacactgaataccaatgctgaactttttaaccagtcagatattgttgctcatctcctttcttcatcttcatctgtcattgatgcactgcagtacaaattagagggcaccacaagattgacaagaaaaaggggattgaagttagccacagctctgtctctgagcaacaaatttgtggagggtagtcataacagtactgtgagcttaaccacgaaaaatatggaagtgtcagtggcaaaaaccacaaaagccgaaattccaattttgagaatgaatttcaagcaagaacttaatggaaataccaagtcaaaacctactgtctcttcctccatggaatttaagtatgatttcaattcttcaatgctgtactctaccgctaaaggagcagttgaccacaagcttagcttggaaagcctcacctcttacttttccattgagtcatctaccaaaggagatgtcaagggttcggttctttctcgggaatattcaggaactattgctagtgaggccaacacttacttgaattccaagagcacacggtcttcagtgaagctgcagggcacttccaaaattgatgatatctggaaccttgaagtaaaagaaaattttgctggagaagccacactccaacgcatatattccctctgggagcacagtacgaaaaaccacttacagctagagggcctctttttcaccaacggagaacatacaagcaaagccaccctggaactctctccatggcaaatgtcagctcttgttcaggtccatgcaagtcagcccagttccttccatgatttccctgaccttggccaggaagtggccctgaatgctaacactaagaaccagaagatcagatggaaaaatgaagtccggattcattctgggtctttccagagccaggtcgagctttccaatgaccaagaaaaggcacaccttgacattgcaggatccttagaaggacacctaaggttcctcaaaaatatcatcctaccagtctatgacaagagcttatgggatttcctaaagctggatgtaaccaccagcattggtaggagacagcatcttcgtgtttcaactgcctttgtgtacaccaaaaaccccaatggctattcattctccatccctgtaaaagttttggctgataaattcattactcctgggctgaaactaaatgatctaaattcagttcttgtcatgcctacgttccatgtcccatttacagatcttcaggttccatcgtgcaaacttgacttcagagaaatacaaatctataagaagctgagaacttcatcatttgccctcaacctaccaacactccccgaggtaaaattccctgaagttgatgtgttaacaaaatattctcaaccagaagactccttgattcccttttttgagataaccgtgcctgaatctcagttaactgtgtcccagttcacgcttccaaaaagtgtttcagatggcattgctgctttggatctaaatgcagtagccaacaagatcgcagactttgagttgcccaccatcatcgtgcctgagcagaccattgagattccctccattaagttctctgtacctgctggaattgtcattccttcctttcaagcactgactgcacgctttgaggtagactctcccgtgtataatgccacttggagtgccagtttgaaaaacaaagcagattatgttgaaacagtcctggattccacatgcagctcaaccgtacagttcctagaatatgaactaaatgttttgggaacacacaaaatcgaagatggtacgttagcctctaagactaaaggaacacttgcacaccgtgacttcagtgcagaatatgaagaagatggcaaatttgaaggacttcaggaatgggaaggaaaagcgcacctcaatatcaaaagcccagcgttcaccgatctccatctgcgctaccagaaagacaagaaaggcatctccacctcagcagcctccccagccgtaggcaccgtgggcatggatatggatgaagatgacgacttttctaaatggaacttctactacagccctcagtcctctccagataaaaaactcaccatattcaaaactgagttgagggtccgggaatctgatgaggaaactcagatcaaagttaattgggaagaagaggcagcttctggcttgctaacctctctgaaagacaacgtgcccaaggccacaggggtcctttatgattatgtcaacaagtaccactgggaacacacagggctcaccctgagagaagtgtcttcaaagctgagaagaaatctgcagaacaatgctgagtgggtttatcaaggggccattaggcaaattgatgatatcgacgtgaggttccagaaagcagccagtggcaccactgggacctaccaagagtggaaggacaaggcccagaatctgtaccaggaactgttgactcaggaaggccaagccagtttccagggactcaaggataacgtgtttgatggcttggtacgagttactcaaaaattccatatgaaagtcaagcatctgattgactcactcattgattttctgaacttccccagattccagtttccggggaaacctgggatatacactagggaggaactttgcactatgttcataagggaggtagggacggtactgtcccaggtatattcgaaagtccataatggttcagaaatactgttttcctatttccaagacctagtgattacacttcctttcgagttaaggaaacataaactaatagatgtaatctcgatgtatagggaactgttgaaagatttatcaaaagaagcccaagaggtatttaaagccattcagtctctcaagaccacagaggtgctacgtaatcttcaggaccttttacaattcattttccaactaatagaagataacattaaacagctgaaagagatgaaatttacttatcttattaattatatccaagatgagatcaacacaatcttcaatgattatatcccatatgtttttaaattgttgaaagaaaacctatgccttaatcttcataagttcaatgaatttattcaaaacgagcttcaggaagcttctcaagagttacagcagatccatcaatacattatggcccttcgtgaagaatattttgatccaagtatagttggctggacagtgaaatattatgaacttgaagaaaagatagtcagtctgatcaagaacctgttagttgctcttaaggacttccattctgaatatattgtcagtgcctctaactttacttcccaactctcaagtcaagttgagcaatttctgcacagaaatattcaggaatatcttagcatccttaccgatccagatggaaaagggaaagagaagattgcagagctttctgccactgctcaggaaataattaaaagccaggccattgcgacgaagaaaataatttctgattaccaccagcagtttagatataaactgcaagatttttcagaccaactctctgattactatgaaaaatttattgctgaatccaaaagattgattgacctgtccattcaaaactaccacacatttctgatatacatcacggagttactgaaaaagctgcaatcaaccacagtcatgaacccctacatgaagcttgctccaggagaacttactatcatcctctaattttttaaaagaaatcttcatttattcttcttttccaattgaactttcacatagcacagaaaaaattcaaactgcctatattgataaaaccatacagtgagccagccttgcagtaggcagtagactataagcagaagcacatatgaactggacctgcaccaaagctggcaccagggctcggaaggtctctgaactcagaaggatggcattttttgcaagttaaagaaaatcaggatctgagttattttgctaaacttgggggaggaggaacaaataaatggagtctttattgtgtatcata

SEQ ID NO: 4 - DNA, 3964 bp, Homo sapiens
  source 1..3964: mol_type = genomic DNA
ctactccaggctgtgttcagggcttggggctggtggagggaggggcctgaaattccagtgtgaaaggctgagatgggcccgaggcccctggcctatgtccaagccatttcccctctcaccagcctctccctggggagccagtcagctaggaaggaatgagggctccccaggcccacccccagttcctgagctcatctgggctgcagggctggcgggacagcagcgtggactcagtctcctagggatttcccaactctcccgcccgcttgctgcatctggacaccctgcctcaggccctcatctccactggtcagcaggtgacctttgcccagcgccctgggtcctcagtgcctgctgccctggagatgatataaaacaggtcagaaccctcctgcctgtctgctcagttcatccctagaggcagctgctccaggtaatgccctctggggaggggaaagaggaggggaggaggatgaagaggggcaagaggagctccctgcccagcccagccagcaagcctggagaagcacttgctagagctaaggaagcctcggagctggacgggtgccccccacccctcatcataacctgaagaacatggaggcccgggaggggtgtcacttgcccaaagctacacagggggtggggctggaagtggctccaagtgcaggttcccccctcattcttcaggcttagggctggaggaagccttagacagcccagtcctaccccagacagggaaactgaggcctggagagggccagaaatcacccaaagacacacagcatgttggctggactggacggagatcagtccagaccgcaggtgccttgatgttcagtctggtgggttttctgctccatcccacccacctccctttgggcctcgatccctcgcccctcaccagtcccccttctgagagcccgtattagcagggagccggcccctactccttctggcagacccagctaaggttctaccttaggggccacgccacctccccagggaggggtccagaggcatggggacctggggtgcccctcacaggacacttccttgcaggaacagaggtgccatgcagccccgggtactccttgttgttgccctcctggcgctcctggcctctgcccgtaagcacttggtgggactgggctgggggcagggtggaggcaacttggggatcccagtcccaatgggtggtcaagcaggagcccagggctcgtccagaggccgatccaccccactcagccctgctctttcctcaggagcttcagaggccgaggatgcctcccttctcagcttcatgcagggttacatgaagcacgccaccaagaccgccaaggatgcactgagcagcgtgcaggagtcccaggtggcccagcaggccaggtacacccgctggcctccctccccatcccccctgccagctgcctccattcccacccgcccctgccctggtgagatcccaacaatggaatggaggtgctccagcctcccctgggcctgtgcctcttcagcctcctctttcctcacagggcctttgtcaggctgctgcgggagagatgacagagttgagactgcattcctcccaggtccctcctttctccccggagcagtcctagggcgtgccgttttagccctcatttccattttcctttcctttccctttctttctctttctatttctttctttctttctttctttctttctttctttctttctttctttctttctttctttctttctttcctttctttctttcctttctttctttcctttctttctttctttcctttctttctctttctttctttctttcctttttctttctttccctctcttcctttctctctttctttcttcttcttttttttttaatggagtctccctctgtcacctaggctggagtgcagtggtgccatctcggctcactgcaacctccgtctcccgggttcaacccattctcctgcctcagcctcccaagtagctgggattacaggcacgcgccaccacacccagctaatttttgtatttttagcagagatggggtttcaccatgttggccaggttggtcttgaattcctgacctcaggggatcctcctgcctcggcctcccaaagtgctgggattacaggcatgagccactgcgcctggccccattttccttttctgaaggtctggctagagcagtggtcctcagcctttttggcaccagggaccagttttgtggtggacaatttttccatgggccagcggggatggttttgggatgaagctgttccacctcagatcatcaggcattagattctcataaggagccctccacctagatccctggcatgtgcagttcacaatagggttcacactcctatgagaatgtaaggccacttgatctgacaggaggcggagctcaggcggtattgctcactcacccaccactcacttcgtgctgtgcagcccggctcctaacagtccatggaccagtacctatctatgacttgggggttggggacccctgggctaggggtttgccttgggaggccccacctgacccaattcaagcccgtgagtgcttctgctttgttctaagacctggggccagtgtgagcagaagtgtgtccttcctctcccatcctgcccctgcccatcagtactctcctctcccctactcccttctccacctcaccctgactggcattagctggcatagcagaggtgttcataaacattcttagtccccagaaccggctttggggtaggtgttattttctcactttgcagatgagaaaattgaggctcagagcgattaggtgacctgccccagatcacacaactaatcaatcctccaatgactttccaaatgagaggctgcctccctctgtcctaccctgctcagagccaccaggttgtgcaactccaggcggtgctgtttgcacagaaaacaatgacagccttgacctttcacatctccccaccctgtcactttgtgcctcaggcccaggggcataaacatctgaggtgacctggagatggcagggtttgacttgtgctggggttcctgcaaggatatctcttctcccagggtggcagctgtgggggattcctgcctgaggtctcagggctgtcgtccagtgaagttgagagggtggtgtggtcctgactggtgtcgtccagtggggacatgggtgtgggtcccatggttgcctacagaggagttctcatgccctgctctgttgcttcccctgactgatttaggggctgggtgaccgatggcttcagttccctgaaagactactggagcaccgttaaggacaagttctctgagttctgggatttggaccctgaggtcagaccaacttcagccgtggctgcctgagacctcaataccccaagtccacctgcctatccatcctgcgagctccttgggtcctgcaatctccagggctgcccctgtaggttgcttaaaagggacagtattctcagtgctctcctaccccacctcatgcctggcccccctccaggcatgctggcctcccaataaagctggacaagaagctgctatgagtgggccgtcgcaagtgtgccatctgtgtctgggcatgggaaagggccgaggctgttctgtgggtgggcactggacagactccaggtcaggcaggcatggaggccagcgctctatccaccttctggtagctgggcagtctctgggcctcagtttcttcatctctaaggtaggaatcaccctccgtaccctgccttccttgacagctttgtgcggaaggtcaaacaggacaataagtttgctgatactttgataaactgttaggtgctgcacaacatgacttgagtgtgtgccccatgccagccactatgcctggcacttaagttgtcatcagagttgagactgtgtgtgtttactcaaaactgtggagctgacctcccctatccaggccccctagccct

SEQ ID NO: 5 - RNA, 533 nt, Homo sapiens
  source 1..533: mol_type = mRNA
tgctcagttcatccctagaggcagctgctccaggaacagaggtgccatgcagccccgggtactccttgttgttgccctcctggcgctcctggcctctgcccgagcttcagaggccgaggatgcctcccttctcagcttcatgcagggttacatgaagcacgccaccaagaccgccaaggatgcactgagcagcgtgcaggagtcccaggtggcccagcaggccaggggctgggtgaccgatggcttcagttccctgaaagactactggagcaccgttaaggacaagttctctgagttctgggatttggaccctgaggtcagaccaacttcagccgtggctgcctgagacctcaataccccaagtccacctgcctatccatcctgcgagctccttgggtcctgcaatctccagggctgcccctgtaggttgcttaaaagggacagtattctcagtgctctcctaccccacctcatgcctggcccccctccaggcatgctggcctcccaataaagctggacaagaagctgctatg

SEQ ID NO: 6 - DNA, 2480 bp, Homo sapiens
  source 1..2480: mol_type = other DNA
tttgcttcccctcttcccgaagctctgacacctgccccaacaagcaatgttggaaaattatttacatagtggcgcaaactcccttactgctttggatataaatccaggcaggaggaggtagctctaaggcaagagatctgggacttctagcccctgaactttcagccgaatacatcttttccaaaggagtgaattcaggcccttgtatcactggcagcaggacgtgaccatggagaagctgttgtgtttcttggtcttgaccagcctctctcatgcttttggccagacaggtaagggccaccccaggctatgggagagttttgatctgaggtatgggggtggggtctaagactgcatgaacagtctcaaaaaaaaaaaaaaaagactgtatgaacagaacagtggagcatccttcatggtgtgtgtgtgtgtgtgtgtgtgtgtgtgtggtgtgtaactggagaaggggtcagtctgtttctcaatcttaaattctatacgtaagtgaggggatagatctgtgtgatctgagaaacctctcacatttgcttgtttttctggctcacagacatgtcgaggaaggcttttgtgtttcccaaagagtcggatacttcctatgtatccctcaaagcaccgttaacgaagcctctcaaagccttcactgtgtgcctccacttctacacggaactgtcctcgacccgtgggtacagtattttctcgtatgccaccaagagacaagacaatgagattctcatattttggtctaaggatataggatacagttttacagtgggtgggtctgaaatattattcgaggttcctgaagtcacagtagctccagtacacatttgtacaagctgggagtccgcctcagggatcgtggagttctgggtagatgggaagcccagggtgaggaagagtctgaagaagggatacactgtgggggcagaagcaagcatcatcttggggcaggagcaggattccttcggtgggaactttgaaggaagccagtccctggtgggagacattggaaatgtgaacatgtgggactttgtgctgtcaccagatgagattaacaccatctatcttggcgggcccttcagtcctaatgtcctgaactggcgggcactgaagtatgaagtgcaaggcgaagtgttcaccaaaccccagctgtggccctgaggccagctgtgggtcctgaaggtacctcccggttttttacaccgcatgggccccacgtctctgtctctggtacctcccgcttttttacactgcatggttcccacgtctctgtctctgggcctttgttcccctatatgcattgaggcctgctccaccctcctcagcgcctgagaatggaggtaaagtgtctggtctgggagctcgttaactatgctgggaaatggtccaaaagaatcagaatttgaggtgttttgttttcatttttatttcaagttggacagatcttggagataatttcttacctcacatagatgagaaaactaacacccagaaaggagaaatgatgttataaaaaactcataaggcaagagctgagaaggaagcgctgatcttctatttaattccccacccatgacccccagaaagcaggagcattgcccacattcacagggctcttcagtctcagaatcaggacactggccaggtgtctggtttgggtccagagtgctcatcatcatgtcatagaactgctgggcccaggtctcctgaaatgggaagcccagcaataccacgcagtccctccactttctcaaagcacactggaaaggccattagaattgccccagcagagcagatctgctttttttccagagcaaaatgaagcactaggtataaatatgttgttactgccaagaacttaaatgactggtttttgtttgcttgcagtgctttcttaattttatggctcttctgggaaactcctccccttttccacacgaaccttgtggggctgtgaattctttcttcatccccgcattcccaatatacccaggccacaagagtggacgtgaaccacagggtgtcctgtcagaggagcccatctcccatctccccagctccctatctggaggatagttggatagttacgtgttcctagcaggaccaactacagtcttcccaaggattgagttatggactttgggagtgagacatcttcttgctgctggatttccaagctgagaggacgtgaacctgggaccaccagtagccatcttgtttgccacatggagagagactgtgaggacagaagccaaactggaagtggaggagccaagggattgacaaacaacagagccttgaccacgtggagtctctgaatcagccttgtctggaaccagatctacacctggactgcccaggtctataagccaataaagcccctgtttacttgagtgagtccaagctgttttctgatagttgctttagaagttgtgactaacttctctatgacctttgaa

SEQ ID NO: 7 - RNA, 1842 nt, Homo sapiens
  source 1..1842: mol_type = mRNA
cgatcagatcgatctaagatggcgactgtcgaaccggaaaccacccctactcctaatcccccgactacagaagaggagaaaacggaatctaatcaggaggttgctaacccagaacactatattaaacatcccctacagaacagatgggcactctggttttttaaaaatgataaaagcaaaacttggcaagcaaacctgcggctgatctccaagtttgatactgttgaagacttttgggctctgtacaaccatatccagttgtctagtaatttaatgcctggctgtgactactcactttttaaggatggtattgagcctatgtgggaagatgagaaaaacaaacggggaggacgatggctaattacattgaacaaacagcagagacgaagtgacctcgatcgcttttggctagagacacttctgtgccttattggagaatcttttgatgactacagtgatgatgtatgtggcgctgttgttaatgttagagctaaaggtgataagatagcaatatggactactgaatgtgaaaacagagaagctgttacacatatagggagggtatacaaggaaaggttaggacttcctccaaagatagtgattggttatcagtcccacgcagacacagctactaagagcggctccaccactaaaaataggtttgttgtttaagaagacaccttctgagtattctcataggagactgcgtcaagcaatcgagatttgggagctgaaccaaagcctcttcaaaaagcagagtggactgcatttaaatttgatttccatcttaatgttactcagatataagagaagtctcattcgcctttgtcttgtacttctgtgttcattttttttttttttttttggctagagtttccactatcccaatcaaagaattacagtacacatccccagaatccataaatgtgttcctggcccactctgtaatagttcagtagaattaccattaattacatacagattttacctatccacaatagtcagaaaacaacttggcatttctatactttacaggaaaaaaaattctgttgttccattttatgcagaagcatattttgctggtttgaaagattatgatgcatacagttttctagcaattttctttgtttctttttacagcattgtctttgctgtactcttgctgatggctgctagattttaatttatttgtttccctacttgataatattagtgattctgatttcagtttttcatttgttttgcttaaattttttttttttttttcctcatgtaacattggtgaaggatccaggaatatgacacaaaggtggaataaacattaattttgtgcattctttggtaattttttttgttttttgtaactacaaagctttgctacaaatttatgcatttcattcaaatcagtgatctatgtttgtgtgatttcctaaacataattgtggattataaaaaatgtaacatcataattacattcctaactagaattagtatgtctgtttttgtatctttatgctgtattttaacactttgtattacttaggttattttgctttggttaaaaatggctcaagtagaaaagcagtcccattcatattaagacagtgtacaaaactgtaaataaaatgtgtacagtgaattgtcttttagacaactagatttgtccttttatttctccatctttatagaaggaatttgtacttcttattgcaggcaagtctctatattatgtcctcttttgtggtgtcttccatgtgaacagcataagtttggagcactagtttgattattatgtttattacaatttttaataaattgaataggtagtatcatatatatgga

SEQ ID NO: 8 - DNA, 18001 bp, Homo sapiens
  source 1..18001: mol_type = genomic DNA
gaagtcatcagcaatgcaactgttcacatggaggatactccctgcttgaggggtcagacaggcctgctgggcaacccaggaggcttggatgaccgtctaccccagtgtttttgggatggaaagttccacattctgagaaccctcagtccctgggcaacctggggtggttagtcaccacagcttgtggcttgggcccatgacagcaggtagaaatgacgtggactgccgccagccgggcacagtggctcacgcctgtaatcccagcactttgggaggctgaggcatgtggatcacttgaggtcaggagttcgaaaccagcctggtcaacacggtgaaaccccatctctgctaaaaaaaaaaaatatatatatataaattagccaggcatggtgacgtgcacctgtggtcccagctactcaggaggctgaggcacaagaatcacttgaacccgggaggtggaggttgcagtgagattgcaccagtgcactctccagcctggcaacagagcaagactctgtctcaaacaaacaaaacaaaacaaacaaaaagacgtaagatgtggaccgctggagaatgggggtgctgcctgcagtcaaaacggagtgggggtgcccagctcagggccagaatgatcctattcccggcacttctcagtgaggctctgtggctcacctaagaaaccagcctcccttgcaggcaacggcctagctggcctggtctggaggctctcttcaaatatttacatccacacccaagatacagtcttgagatttgactcgcatgattgctatgggacaagttttcatctgcagtttaaatctgtttcccaacttacattaggggtttggaattctagatcgtatttgaagtgttggtgccacacacaccttaacacctgcacgctggcaacaaaaccgtccgctctgcagcacagctggggtcacctgacctttctcctgtcccccccacttgagctcagtggctgggcagcaggggatgcatggccactggccggccaggtgcagctctcagctggggtgttcagaggacgcctgtgtcctcccctcccccatccctctgtcacccttggaggcagagaactttgcccgtcagtcccatggggaatgtcaacaggcaggggcagcactgcagagatttcatcatggtctcccaggccctcaggctcctctgccttctgcttgggcttcagggctgcctggctgcaggtgcgtccggggaggttttctccataaacttggtggaagggcagtgggcaaatccaggagccagcccgggcttcccaaaccccgcccttgctccggacacccccatccaccaggagggttttctggcggctcctgttcaatttctttccttctagaaaccagcatccaggcacaggaggggaggcccttcttggtggcccaggctttggcgggattatttttcaaagaactttaggagtgggtggtgctttcctggcccccatgggcccctgcctgtgaggtcggacaagcgcagggagtctggggcctctcagagtgcaggaagtgcgcacagggtgctcccaggctggggagcacaggtaggggacggtgcgtgggggatggcgcctggggcatgggggatggggtgtgggaaacggcatgtggggcgtaggggatggggtgtggaggatcgggggtggggatggcgtgtggggtgtgggggatgggccgtgggggggtggggcctgggaaacagcatgtggggcatggggtgtgggggtgaggtgtgggaaagtgtgtggggtgtgggggatggggcatggaaagggcgtgtggggtgcaggggatggggcatggaggtgtgggggatggggtgtgtggggtgtcggggatggggcatgtggggtgtgggggatggggcatggaaagggcgtgtggggtgcagaggatggggcatgggggggtggggatggcgagtggggctggggcctgggaatggtgagtggggcatggggatggcgagtagggggtgtggcgtgaggatggctagtggggcgtggggatggcgtgtggggatggcgagtggggggtgggctgtgagggacagtgcctgggatgtggggctgcagccctagctcacagcatggccttatgaccccggccaccttcctgccccaggcggggtcgctaaggcctcaggaggagaaacacgggacatgccgtggaagccggggcctcacagaggtgagcagggactgccactggttttgtcctggggcccagtgggggccaacatcacctccttcccctcccatggcaaagagccagcccgcggggtggctactgcagtgccccccaaggagggtgttccctgctcgagaggaagtgaccgctccagcttggccttccctgggactggggtgcaggcgattttatcttctttgctccattctgttccttccagataatcgtgtgttcttcatcaggttttcctcagttcttgagagcttttctgatgcaaatctgctttcaccccagggcggtcaccggctctgctcacaccagcctccaagggtgtgggtgtcccgggagtgtgggtgtcccgggggcgtgggtgtcccaggagtgtgggtgtcccgggggcgtgggtgtcccgggagtgtgggtgtcccgggggcgtgggtgtcccgggagtgtgggtgtcccggaggcgagggtgtcccgggagtgtgggtgtcccgggggagtgggtgtcccgggagtgtgggtgtcccggaggcgagggtgtcccgggagtgtgggtgtcccgggggcgtgggtgtcccgggagtgtgggtgtcccgggggagtgggtgtcccgggagtgtgggtgtcccggaggcgagggtgtcccgggagtgtgggtgtcccgggggagtgggtgtcccgggagtgtgggtgtcccggaggcgagggtgtcccgggagtgtgggtgtcccgggggcgtgggtgtcccgggagcgtgggtgtcccgggggcgtgggtgtcccgggagtgtgggtgtcccgggggcgtgggtgtcccgggagtgtgggtgtcccgggggcgtgggtgtcccgggagtgtgggtgtcccgggagtgtgggtgttccggaggcgagggtgtcccgggagtgtgcgtgtcccgggggcgtgggtgtcccgggggcgtgggtgtcccgggggcgtgggtgttccggaggcgagggtatcccagaagtgtgagtgtcccaggggcgtgggtgtcccgggggtgtgggtgtcccgggggcgtgggtgtcccgggagtgtgggtgttccggaggtgagggtgtcccgggagtgtgggtgttccggaggcgagggtgtcccgggagtgtgggtgtcccgggggcgtgggtgtcccgggagtgtgggtgttccggaggtgagggtgtcccgggagtgtgggtgttccggaggcgagggtgtcccgggagtgtgggtgtcccaggggcgtgggtgtcccgggagtgtgggtgttccggaggcgagggtgtcccgggagtgtgggtgttccggaggcgagggtgtcccgggagtgtgggtgtcccgggggcgtgggtgtcccgggggttgtgggtgtcccgggagtgtgggtgttccggaggcgagggtgtcccgggagtgtgggtgttccggaggcgagggtgtcccgggagtgtgggtgtcccgggggtgtgggtgtcccgggggtgtgggtgtcccgggagtgtgggtgtcccgggggagtgggtgtcccgggagtgtgggtgttccggaggcgagggtgtcccaggagcgtgggtgtcccggaggcgagggtgtcccgggagcgtgggtgtcccgggggcgtgggtgtcccgggagtgtgggtgtcccgggggagtgggtgtcccgggagtgtgggtgtcccggaggcgagggtgtcccaggagtgtgggtgtcccgggggcgtgggtgtcccgggagtgtgggtgttccagaggcgagggtatcccagaagtgtgagtgtcccgggggtgtgggtgtcccgggggtcgtgggtgtcccgggagtgtgggtgttccagaggcgagggtgtcccgggagtgtgggtgtcccaggggtgtgggtgtcccgggggcgtgggtgtcccgggagtgtgggtgtcccgggggagtgggtgtcccgggagtgtgggtgttccggaggcgagggtgtcccgggagtgtgggtgttccggaggcgagggtgtcccgggagcgtgggtgtcccgggggcgtgggtgtcccgggagcgtgggtgtcccaggggtgtgggtgtcccgggggcgtgggtgtcccgggagtgtgggtgtcccgggggagtggatgtcccgggagtgtgggtgttccggaggcgagggtgtcccgggagtgtgggtgttccggaggcgagggtgtcccgggagtgtgggtgtcccgggggcgtgggtgtcccgggagtgtgggtgtcccgggggcgtgggtatcccagaagtgtgagtgtcccaggggcgtgggtgtccggggggcgtgggtgtcccgggggtgtgggtgtcccgggggtcgtgggtgtcccgggagcgtgggtgtcggggactgcagggacatgggcctcccctcccactcctgccgcccagggcacctcctgtgaggactcggagtccgtgagttcccacctccttgagcccgattctttggtgtccccgcctgcatcctcagcctccttccaaaccagaccagttctctaggggcgtcgacgtgtgaaactgattttaaagaaaacaggcagtggcctttctctcggccccacgtggcccagtagcgctcaccttccgtcccttcttccgcgctcagtaaccaatttaggccgctcctgcagaactcgggctcctgcccaccggcccacagcgtccacctgaggcctcgtcctcccagcaaaggtcgtccctccggaacgcgcctcctgcggcctctccagagcccctcccgcgcgtcctctcagccccgctcgcctcctcccggggcctccctctcccgcctgcccccaggcccgtctcccctcgcgggctgaggcaggttcgggcagcacggccgccccggggcgggggtcactctccaccaccgcgtggtgcccacagctcacggcgctcccgggtgacggtcccctcggctgtagggcgtcctgaagagcggcctgctcggagctgagcgcacggggttgcctcgccctgggcgtctctggccctcaccagccccgtcttcccatgggcaaaacggcggtcctgtttgtccacaagtaaccgtcggggttacggaggggccaggagctgcggcggggggctgtgctctcaggaccggccccaggaggatccgcgcgaggtctggagctctcaggggtcgcgggggacagaggggccccaagcggaggcggggaaggcggcagaagcccaggaccgccaagagctggcgaggaagcccggggctcgctgtcgggggagccgggcaggggccgcgcctcggaccaggacggaggcctggggaaggcggatctggccgccggagacgcggtgcgggtggagacgagggatttggatttccgcgggcggctgtacggatttccacgcgcggttcacgtgggccccagggggttgcccggcacccggggccgcgccgccttctcctcgccggcatcgacccgcagcctcacgtttacgcggcggcgcccgcagcccccttcggcccggcttccgcgcgtgcccccgagcgcgccctcgggatcagcccccggaagcagagaggccaggccgggaaggatgggcgacgggggtggctgacccgggagcacggcagggaggacacccagccaggcccgcgagcagcgccgctcccctcctccaggacgggcgggaacctgcgatgcccccgccgcgtgggccgtggggcggtctccgaggcactgggcggggcacgcggtgggcgcttcacggaactcgcatttcccagtcttcgtaacccaggaggaagcccacggcgtcctgcaccggcgccggcgcgccaacgcgttcctggaggagctgcggccgggctccctggagagggagtgcaaggaggagcagtgctccttcgaggaggcccgggagatcttcaaggacgcggagaggacggtgagcccagcctcggggcgccccgcgccgcggacactgcaggcggcggtgaaccaggccgcgtggggccgcctgcgtctctttggctgcggctgtgggcggcgaacacgcagcggcgcccgcgcggcgctttctgcgggggtcgctttccgcccggggtgactccgctttcctgggcgatgccccccacccccaggcacgcgctctccccgtgcggccgcaccgcgcatgccggttttcacatcagaaaatacgatttgcaaagcacacttagggtgtcccccttaacttcccaagggagtccccccagtccccgaagggtccagggcagcctgcgcatcgcagacgcgcgcggctcgcagaagggacgtggtgagaagctggcccacagcatgccaccagcggcacctcctcagggcacgtgtcggggagaaacaacacttagggacctgggactttctccagctcacgctcacgggtcacctcacactccaagatcacctcaaagaggacacctcacacagggcacacttcacactcacaggtcacctcacactcacaggacacctcacactcacagggcacacttcacactcacgggtcacctcacactccaagatcacctcaaagaggacacctcacacagggcacacttcacactcacaggtcacctcacactcacaggacacctcacactcacagggcacacttcacactcacgggtcacctcacactccaagatcacctcaaagaggacacctcacacagggcacacttcacactcacgggtcacctcacactcacaggacacctcacacaagacacctcacacggggcacacttcacactcacaggtcacctcacacccacaggacacctcacacagggcacacttcacactcacgggtcacctcacactcacaggacacctcacacaagacacctcacacggggcacacttcacactcacaggtcacctcacacccacaggacacctcacacagggcacacttcacactcacgggtcacctcacactcacaggacacctcacactcagggcgcacttcacactcacgggtcacctcacacccacaggacacctcacagaggtcacctcacacaggacacctcacactcagggtgcacttcaaacccacaggtcatttcacctcacactcacaggacacctcacacaagataccacacggggcacacttcacactcacaggtcacctcacactcacaggacacctcacagaggtcacctcacacggggcacacttcacactcacaggtcacctcacacccacaggacacctcacagaggtcacctcacacccacaggacacctcacacaggacacctcacagaggtcacctcacacccacaggacacctcacactcataggtcacctcagtcttacaggacaactcacactcacaggtcaccttactctcacaggacacctcacactcacaggtcaccttactctcacaggacacctcactctcacaggacacctcacacagggcacacttcactcccacaggtcaccatacctcacacagatcacctcatactcacagatcacttcattctcacaggatacctcacactcagggcacacttcacactcacaggtcacacctcacacagatcatctcattctcacaggacacctccctctcacaggtcacctcacactcacaggacacctcacagaggtcacctcacacccacaggacacctcacagaggtcacctcacacggggcacacttcacactcaggtcacctcacacccacaggacacctcacagaggtcacctcacacccacaggacaactcacagaggtcacctcacacaggacacctcacaaaggtcacctcacacccacaggacacctcacactcataggtcacctcagtcttacaggacaactcacactcacaggtcaccttactctcacaggacacctcacactcacaggtcaccttactctcacaggacacctcacacagggcacacttcactcccacaggtcaccatacctcacacagatcacctcatactcacagatcacttcattctcacaggatacctcacactcagggcacacttcacactcacaggtcacacctcacacagatcatctcattctcacaggacacctccctctcacaggtcaccttacactcatctcacactcacaggtcgccacacctcacactcacaggatgcctcacactcacagaaccacatctcatatgcacaagacacctcacactcaggacacctcatgctcaaagaagcctcacactcacaggaggtccagctgtctgaggcaaaggctaacatgaccctttccagacaaattgaggatggtcatgcctagcatttttatacacctagttttgaaagcatttctcatctgttgtattctcacagcaccccgtgagtttaagttcaggtggccaacagtttcttcagcaatcacttttttctgtggagtgcttttgctgtttgtggaatattttgcatctgctactgcaccctctccccgtatgtgtggccaccctgtcagaggtggagctgtggctcagagcctgtgtacctcgtcccaggtccacagctcagcgacagaagagtcagggttgaacctcgggtgttctgacttgggagcaggaaatgtgtggtcacccatagttccagatgtcctggggaggggccaagattagaagaaacctacctcagctccagaggaaagtctggcttcctgagcccaccccgccagacccaggtccaagtcccccaaccccagttcatggtgtgtccagtgcttaccgttgggtgctctggtgaaggtgcatctcacgaggcttgctctcttgttccttcagaagctgttctggatttcttacagtggtgagtggatgatcaccaccagtcctgcctgcaacccttctcagcttactgacaccagcccactccacagatggggaccagtgtgcctcaagtccatgccagaatgggggctcctgcaaggaccagctccagtcctatatctgcttctgcctccctgccttcgagggccggaactgtgagacgcgtaaggccccactttgggtcccatatttgcagagggccctggggagctggtggaggtggcctggccaaccgggctgcagggtgcacaacctggtggggtgtgtaggccgggcattcagggctcagccccagttggaaattggtctaggtgaccttgaaatcccttccagtctgaggtctttgacagggacccaaggttctgattatcagactcagtggccccttgggctcccggccctgggcaattctcagccctcgagatggcccagctgagagtccctgtgtccctgtcccacttccacatcccaccacgcaggaccgcttggtaaacttccccttctctactttccattacaaaggtttgaggggtttgttttttttttaaccatctgaatattaaattatcacaaagtttgaggcccccaacctcccttgggttcagtaattcactagaaggactcatagaatccactgaagtggatacactcacaggtaccgtttattacagcaaaggatgcaggcttaagtctgcagagggaccaggcacaagcttccccttgtcctctccctgtggggtcatgtggacagtccttaattctcccagaatgacgtgtgacgagacgtgggaagtactgccaacttgggaagctctacgagccccggtgtccagaggttttatcagggctcaatcacatagacccagctgaccacccgcatggctgacctcagtctcagcccctccagaggctacgccgatagtgcggcccaaggccccaccatacatcacattgtcagctagaccatccagcatggctcaaggcccaggtaaacaccaacattccctcaggcaagaccttccaagggcttagcggtcatttcccaggagccaaggcaaaggctaccctttctctggcacagcagttcatccttgaccacccaagaccacattcttacactgaatgagctctcctgtgcagcagccattttcttctctaagcagaagagagcccagcaagctggaggaggctgaagagagaggcttcctgctggtcatctgggtccagaatgcctggagatctctgctcagccctggtgcccagcagccctggtgtgcatcctgcagggcaggccttcccgccggagtcctggacttgctcagggccactccccttgcccatgtcaaccaaagtcaggctgccggttctgcttcttctgtctgagcccatgaccagtgctgggactaactgtccccaggcgggctcacggtggtacgaggccagcttggagaactgtctcagctctctggtcctctcgtcagttgggtctctgattggaaagtcccttggacacttttaccatccccattggactttcactttcccccaggctcccatcagctgctcggaagagtggtcaccctggaggccactgcccaccagccaggcaccccccaaatgcaaccgcagccagcactgccagccactggcaaggctgttcagacatgtggctcctctgatccacgccttgtcctttggatcagtccacggagcaggtggtgccaagctcaggctctgtcacccacagctcagtgccaccttccaggcagaacaccactgctgacccagggcatggccaccccgggggctggctctcgctgacccccagaagcccctctcagggtgtccccttcctgtccccagacaaggatgaccagctgatctgtgtgaacgagaacggcggctgtgagcagtactgcagtgaccacacgggcaccaagcgctcctgtcggtgccacgaggggtactctctgctggcagacggggtgtcctgcacacccacaggtgaccaggcttcatgtcccagtcccagatgacaccagtccctgtcccactacggattatcttactggacaaaagacgggtgggagtggcttcacatctactgagcactaactatgcactgaccaattgtgaggtgggatctgggcaccaagggtggcacaggccagagcgacagtgactaggatgggcaccctgggggcaatccctgaatggcctcaggccccctgccaattctaggcagaccaggggagccaagcaaggcactatctcacgtccaactcccactcgcaggacctccgccagggttcatgaatctacttcggcacagccaatgtctgtactgactgctgcccactctgcattccaaaactcgtaaaggctcctgggaaaatgggatgtttctccaaaccagcctggaacgaatgggctgcacttccaaaagcagggacaccccacacccactgtgtctcaaagaggcggacgtgcccaccctggccacacagcctgggactcagcctgccacctcctcgggcttcctttctggcccaagaccttgattgaagcagatcaaaactaagcatgggatcaaaacaacacagtttgattcatctttaggtagaatttcattcaccttctactaaagtcaaacaacacatcttctccctgaaaagtgagcagagggcggttttaagacgtaagccctctgtttcctccaaaaccagccctgaccattgtctcctcagccagccacttcttcaagggcctctcatggccgggccccaccagtcaggcccagccgaggccctgccttccaccacccctgggccctgggagctcctgctcctgggggcctcccatagcctcggcctcaaggcctctcagaggatgggtgtttctgaatctttcctagtggcacgttcatccctcacaaatctctgcatctttctgacttttgttttacacagttgaatatccatgtggaaaaatacctattctagaaaaaagaaatgccagcaaaccccaaggccgaattgtggggggcaaggtgtgccccaaaggggagtgtccatggcaggtaaggcttcccctggcttcaggattccaagccctgagggtcttgaagccttttgaatgtgaacaacagctctggaagggaaaatgggcaggtcagccccaagcccaccaggctccaagtcagcacacctagcacctccagctcgcggcacccccatgcttttagtggggcaaggaaggagaaaagaaaacgacactcactgagggtctaccctgtgcagagaaccctgcgagatgccccatccgagttgtcacgtcgtcctcacggttactctttgaggtgggatctttgcctgatctttgcaaaatcaggagcattggatcaaagctatgtgaagatcctgtgaggtgaacagtgaaatctcacagcgacatttgtattcttgggccgtgcccaagagcacgtctcggctagagaggggcacagcctcccagagccaggtctgagcagctttgcctgggagggatctgcaaagaccccaggatttcagaaagaaattgtgcaatgccagaggttccttggcatgcccgggagggcgagtcatcagagaaacaatgacagcaatgtgacttccacacctcctgtccccccgcccaggtcctgttgttggtgaatggagctcagttgtgtggggggaccctgatcaacaccatctgggtggtctccgcggcccactgtttcgacaaaatcaagaactggaggaacctgatcgcggtgctgggtgggtaccactctcccctgtccgaccgcggtgctgggtgggtgccactcttccctgtccgaccgcggtgctgggtgggtgccactctcccctgtccgaccgcggtgctgggtgggtgccactctcccctgtccgaccgcggtgctgggtgggtgccactctccgctgtccgaccgcggtgctgggtgggtaccactctcccctgtctgaccgcagctctcaagtgtctcaggggctgtggctctgggcttcgtgctgtcacttccacagacagacagacatccccaaaaggggagcaaccatgctgggcacgactgctgtggccaccgtgctctcagccactttcccatgcccaaataaaacgataaaagactgggggcttctgcccatcctgcctcacttgaccaagagcccagaagaggatgcgacacccagggcctcatgggaccaccggctggcaggggttctgctcactgggtttatgggtgagacgagcactcccaggagggccactgggccgggaagaactgtggagaatcggggcacgccctgtcctcccagctgccagggcacagcatcccttccccacctcaacacccagaccccagattcaccccagttcacttgtccccacacgagccacaggctgccacctggggcaggctggccccaccttggggttagatgcaggtccccttgccccagaaggagactgcagcccctgcagacctagaaatggccacagcccatccccatgcaccagggggtgaggtggcaggtggtggaaagggcctgaggggggcttcttccttccaggcgagcacgacctcagcgagcacgacggggatgagcagagccggcgggtggcgcaggtcatcatccccagcacgtacgtcccgggcaccaccaaccacgacatcgcgctgctccgcctgcaccagcccgtggtcctcactgaccatgtggtgcccctctgcctgcccgaacggacgttctctgagaggacgctggccttcgtgcgcttctcattggtcagcggctggggccagctgctggaccgtggcgccacggccctggagctcatggtcctcaacgtgccccggctgatgacccaggactgcctgcagcagtcacggaaggtgggagactccccaaatatcacggagtacatgttctgtgccggctactcggatggcagcaaggactcctgcaagggggacagtggaggcccacatgccacccactaccggggcacgtggtacctgacgggcatcgtcagctggggccagggctgcgcaaccgtgggccactttggggtgtacaccagggtctcccagtacatcgagtggctgcaaaagctcatgcgctcagagccacgcccaggagtcctcctgcgagccccatttccctagcccagcagccctggcctgtggagagaaagccaaggctgcgtcgaactgtcctggcaccaaatcccatatattcttctgcagttaatggggtagaggagggcatgggagggagggagaggtggggagggagacagagacagaaacagagagagacagagacagagagagactgagggagagactctgaggacatggagagagactcaaagagactccaagattcaaagagactaatagagacacagagatggaatagaaaagatgagaggcagaggcagacaggcgctggacagaggggcaggggagtgccaaggttgtcctggaggcagacagcccagctgagcctccttacctcccttcagccaagcccacctgcacgtgatctgctggcctcaggctgctgctctgccttcattgctggagacagtagaggcatgaacacacatggatgcacacacacacacgccaatgcacacacacagagatatgcacacacacggatgcacacacagatggtcacacagagatacgcaaacacaccgatgcacacgcacatagagatatgcacacacagatgcacacacagatatacacatggatgcacgcacatgccaatgcacgcacacatcagtgcacacggatgcacagagatatgcacacaccgatgtgcgcacacacagatatgcacacacatggatgagcacacacacaccaatgcgcacacacaccgatgtacacacacagatgcacacacagatgcacacacaccgatgctgactccatgtgtgctgtcctctgaaggcggttgtttagctctcacttttctggttcttatccattatcatcttcacttcagacaattcagaagcatcaccatgcatggtggcgaatgcccccaaactctcccccaaatgtatttctcccttcgctgggtgccgggctgcacagactattccccacctgcttcccagcttcacaataaacggctgcgtctcctccgcacacctgtggtgcctgccacccactgggttgcccatgattcatttttggagcccccggtgctcatcctctgagatgctcttttctttcacaattttcaacatcactgaaatgaaccctcacatggaagctattttttaaaaacaaaagctgtttgatagatgtttgaggctgtagctcccaggatcctgtggaattggatgttctctccctgccacagcccttgtcaatgatatttcacagagaccctgggagcacctgctcaagagtcagggacacacgcatcactaaatgcaagttcccaggccctggctgcagtgggaggacctggcaagctgcactcttgctgagtccccagggtggtggaagaagaatgagaaacacatgaacagagaaatggggaggtgacaaacagtgcccccactcagactccggcaagcacggctcagagagtggactcgatgccatccctgcagggccgtcctgggcaccactggcactcacagcagcaaggtgggcaccattggcactcacagcagcaaggcaggcaccagcaacccacctcgggggcactcaggcatcatctacttcagagcagacagggtctatgaactacagccgtgggctgcttccaaggcaccctgctcttgtaaataaagttttatgggaacacacccatattagtgtccatggagtggccgtggcagagacgtccagccggacagaccagctgacccgccaagcccagcatggttagtgtcaggacctctgctgaagatgcttgctgaccctggccagaccccggttcctaatgccccctaaacgggacgggagccagtggcgggccctgatccaggtcagagctggctctgctttctcttttgtccgagtgaccatgcctcagtttcctcatgtgtaaaacaggagcccaccgtgatgcttatggtgggatgagatcagcatggatggaacaaggccctggaagggcccatgccatggtcatcgacagcaaagccactctgcagacagatgcttcagtgaattggtagaaaattctgcaaccagaatgcccggggctcctgagggcctaagcccagcccagggttctggaagccactctgacttcttgggagtggaagttggcaggactcttcctgggaagaagcggagggtggggatgagaggacagttcaggagcccacccagacccacaggaggaaactaggggagtcatgcggggtcctggtggagcgccagcctcccttcctgccaatgggaaatgcaggcgcccacctcatggtgctgccggaggagggggcccgggactccccagaggcttcgctgaagggcctgggcgcccccaaaggctacatgtttcatatgggacgtgccacctgccacggctcagctccagctttctgtgagtggcgagatagaatacggggaggccactggccatgggcctgggacagggtgggatgaggcggcaggcttgggccaccaaagccagcatcgccacccagcattgatgacaaagactgcgtgtctgccatgagcatcctgctgttggtgcacacaccgcattggtctctccatacaaacatgcctagaggcgatgtcagagggtggagaccaggagaggcaggagtcagacatctggtgccaccaggaaggcccttctcagaggaccaggctgtgcgtggtgcccgccgtgggaggccagcctggcgttggcatccagcatcatcagtttgtgcagtcgggtggggctcagtgagtgcctcctgtgtgccaggcacaatgacgcacaatgtgtgcacaccaggctcatgtgcaggtggctgcgagacagggcgacccatcaaggcagatgcaccatgaggcagtggccagtgctgtgggtgttaggggcattgctccccggccactacggcatagcaggcagtgatcgccacactggccaagctttagaccatttattccagagaccccagaggcaaaaagcccggctgcacctcccagtgactcccacagccattgagcagagacactcaggaccttgtgatgggaggtttctgcactggagaacgagcccagaagccctctcagcctcggaacagtgtggccagtggtgggcaggtcaggaggggcttcagacacagcctgtccctccagatggtcacgggaaggtcactccccacagaagtacgttttggggccatgcgggcacagaaggtttgggggtgggtggggcaggtgccagcctggcctgtgggaggccatggtgcagatgccaagccccccccgtgacatgagaccacctgataccacccagagagtggctgtgagcggaagggcccgcccagaaacaagcagggccttggggcagaagtcctgggctcagatcccacgctcactgccagcggcctcggctcaggcttctgcgctctctaaacttagttttctcttctggaaaaatgatggggaaaatgatatttgtatgtgaggactgagagttaaatgtaaacatctggaaactacaaaatgagcacgaaatgatgtttttattcttagaacagaaagtccccacacccgcggccctggtgactgatgaggatgaggttctgcggggcctctctggccgcccagctctgcctggggaaggtggggcca

SEQ ID NO: 9 - RNA, 3278 nt, Homo sapiens
  source 1..3278: mol_type = mRNA
aggcacacaggcaaaatcaagttctacatctgtccctgtgtatgtcacttgtttgaatacgaaataaaattaaaaaaataaattcagtgtattgagaaagcaagcaattctctcaaggtatatttctgacatactaagattttaacgactttcacaaatatgctgtactgagagagaatgttacataacattgagaactagtacaagtaaatattaaagtgaagtgaccatttcctacacaagctcattcagaggaggatgaagaccattttggaggaagaaaagcacccttattaagaattgcagcaagtaagccaacaaggtcttttcaggatgattttcttatatcaagtggtacatttcattttatttacttcagtttctggtgaatgtgtgactcagttgttgaaggacacctgctttgaaggaggggacattactacggtcttcacaccaagcgccaagtactgccaggtagtctgcacttaccacccaagatgtttactcttcactttcacggcggaatcaccatctgaggatcccacccgatggtttacttgtgtcctgaaagacagtgttacagaaacactgccaagagtgaataggacagcagcgatttctgggtattctttcaagcaatgctcacaccaaataagcgcttgcaacaaagacatttatgtggacctagacatgaagggcataaactataacagctcagttgccaagagtgctcaagaatgccaagaaagatgcacggatgacgtccactgccactttttcacgtacgccacaaggcagtttcccagcctggagcatcgtaacatttgtctactgaagcacacccaaacagggacaccaaccagaataacgaagctcgataaagtggtgtctggattttcactgaaatcctgtgcactttctaatctggcttgtattagggacattttccctaatacggtgtttgcagacagcaacatcgacagtgtcatggctcccgatgcttttgtctgtggccgaatctgcactcatcatcccggttgcttgttttttaccttcttttcccaggaatggcccaaagaatctcaaagaaatctttgtctccttaaaacatctgagagtggattgcccagtacacgcattaaaaagagcaaagctctttctggtttcagtctacaaagctgcaggcacagcatcccagtgttctgccattcttcattttaccatgacactgatttcttgggagaagaactggatattgttgctgcaaaaagtcacgaggcctgccagaaactgtgcaccaatgccgtccgctgccagttttttacctataccccagcccaagcatcctgcaacgaagggaagggcaagtgttacttaaagctttcttcaaacggatctccaactaaaatacttcacgggagaggaggcatctctggatacacattaaggttgtgtaaaatggataatgagtgtaccaccaaaatcaagcccaggatcgttggaggaactgcgtctgttcgtggtgagtggccgtggcaggtgaccctgcacacaacctcacccactcagagacacctgtgtggaggctccatcattggaaaccagtggatattaacagccgctcactgtttctatggggtagagtcacctaagattttgcgtgtctacagtggcattttaaatcaatctgaaataaaagaggacacatctttctttggggttcaagaaataataatccatgatcagtataaaatggcagaaagcgggtatgatattgccttgttgaaactggaaaccacagtgaattacacagattctcaacgacccatatgcctgccttccaaaggagatagaaatgtaatatacactgattgctgggtgactggatgggggtacagaaaactaagagacaaaatacaaaatactctccagaaagccaagatacccttagtgaccaacgaagagtgccagaagagatacagaggacataaaataacccataagatgatctgtgccggctacagggaaggagggaaggacgcttgcaagggagattcgggaggccctctgtcctgcaaacacaatgaggtctggcatctggtaggcatcacgagctggggcgaaggctgtgctcaaagggagcggccaggtgtttacaccaacgtggtcgagtacgtggactggattctggagaaaactcaagcagtgtgaatgggttcccaggggccattggagtccctgaaggacccaggatttgctgggagagggtgttgagttcactgtgccagcatgcttcctccacagtaacacgctgaaggggcttggtgtttgtaagaaaatgctagaagaaaacaaactgtcacaagttgttatgtccaaaactcccgttctatgatcgttgtagtttgtttgagcattcagtctctttgtttttgatcacgcttctatggagtccaagaattaccataaggcaatatttctgaagattactatataggcagatatagcagaaaataaccaagtagtggcagtggggatcaggcagaagaactggtaaaagaagccaccataaatagatttgttcgatgaaagatgaaaactggaagaaaggagaacaaagacagtcttcaccattttgcaggaatctacactctgcctatgtgaacacatttcttttgtaaagaaagaaattgattgcatttaatggcagattttcagaatagtcaggaattcttgtcatttccattttaaaatatatattaaaaaaaatcagttcgagtagacacgagctaagagtgaatgtgaagataacagaatttctgtgtggaagaggattacaagcagcaatttacctggaagtgataccttaggggcaatcttgaagatacactttcctgaaaaatgatttgtgatggattgtatatttatttaaaatatcttgggaggggaggctgatggagatagggagcatgctcaaacctccctaagacaagctgctgctgtgactatgggctcccaaagagctagatcgtatatttatttgacaaaaatcaccatagactgcatccatactacagagaaaaaacaattagggcgcaaatggatagttacagtaaagtcttcagcaagcagctgcctgtattctaagcactgggattttctgtttcgtgcaaatatttatctcattattgttgtgatctagttcaataacctagaatttgaattgtcaccacatagctttcaatctgtgccaacaactatacaattcatcaagtgtg

SEQ ID NO: 10 - DNA, 10 bp, Homo sapiens
  source 1..10: mol_type = genomic DNA
aaaaaaaaaa

SEQ ID NO: 11 - DNA, 20001 bp, Homo sapiens
  source 1..20001: mol_type = genomic DNA
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntacttggccctcctaattgggtgttctcagtgaaaacgaggacactgctaatatgctttagaaaatagccctcacattctccctgttcccaatcccccacttactctaagctccccaggtagcaataattcagaagtcaaattgctcagcactcctatggttcaagtgattcttgtgtctcagcctcccaagtagctgggactacaggagcccaccaccacgcccagttaatttttgtattttttagtagagatggggtttcaccatgttgaccaggctggtctcgaactcctgacctcaagtgatccactggcctcggcctccaaaagtgttgggattacaggcgtgagccactgcgcccagcctcaaccttctagtgaaccctccatgctctgttatcttttattcctcttggatttttgttgtttcttttctttttcttcttctttttcttttctttttttttttttgagatggagtttcattcttgttgcccaggctggagtgcaatggcacaaccttggctcactgcaaccttcgcctcctgggttcaagcaatttgcctgcctcagcctcccaagtagctgggattacaggcatgtgctaccatgcctggcgaattttgtatttttagtagagacagggtttctccgtgttggtcgggctgatctcaaactcccgacctcaggtgatcagcccgccttggcctcccaaagtgctgagattacaggcatgagccaccacacccagcctttttgttgtttcttctgagagatttcttcaactcaattttccaacccttctattaaattttttaaattccagatattctatttgcagccggcaaggactcttcctgcgctctgcttgttttccaaagcattccgttctagtttttatgggagcatcatcctttcatgtctctaaggataatcagagtggttaaaatgttcttgaagttttcttctgttccctgcagtagctctgtttcctccagtttcctctttcccaagtgattggtctgtctcatatacctagaggtcttgctttgcattcacatctaagggcaaaaggcgctaggatgcagtgcggaggtccattcgctttgtcgtaaggtttgtgcctttcttagtcctgcagtggttgagtaaaacctgaccatcccacaccctcaaatactaagtgcccctgggtagtgatgtggaggggccttcttattaatgtgaggaaatgcttgtgttataggttgtggtgagaaacgctggttacaaaactatatcaaagtaaaaatgtattaatgcacagtaaagacacctggaaaaaaaatgccctttaatgctcacagaaggtctctccgaggggcagccccaccaccctcctgtttcccttcctgcatttccacgtttttctgggcccagatgcagcctcccctcccacccctggtccctccgccttggcttccggctgtcgctttcatccctcctcctcatcagccccttgcagaactccagggtggggcttctgagtctcgctggcagtatgggctccataagtcttgctggacaccgaaattaagttctgcaggtgccgtctccagaatccccagcacagatagacaaacccacatctcaggggtggggggtgcagacctgcccccagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntcccagctgccctctgtttatatgtctcaccaatgtcaagggaaaccagaactggatagcagttgaaacacatattttgttcgggactattgtaataggggaaaaaagattttagtatagacctgggctcaactctcaatgtggcacaggcaagtggggatttagatctgaggagcagggcggggtcagtgggtggaaaattactggcacgaaacacctgtctggaggattctggctaaacccaggaaacaggaagcttgctgagggcaggcagggttagcagacatcgcctgggggtggcggaggctgagaaccctacccaggtaaaatgaagcttgctgacggcagacagggttagcagacacggcctggaggtggcagaggctaaggaacctacccaggtaaaacgaagcttgctgacggcagacagggttagcagacacggcctggaggtggcagaggctaaggaacctaccaaggtaaaatgaagcttgctgaaggcagccagggtgagcagatattgcctgggggtggcggaaactgaggaccctacccaggtaaaaggaagcctgctgaaggcaggcagggtgagcagacattgcctgtgggtagcagaggctgaggactctacccaggtaacaggaatcttgcttaaggcaggcaaggtgagcagacatcgcctgggggtggcggaggctgaggactctacccagataacaggaatcttgctgcgggcagccagggtgagcagacgtcgcctgggggtggtggaagctgaggaccctacccaggtaaaaggaagcttgctgaaggcaggcaaggtaagcagacatcgcctgggggtggcggaggctgaggaccctgatcagatatggggggatggaggcttcttgccaaactgacttagcagagttcttgctgaatctggattttataaggcagaatgcagatgagcctgtgagaaggttctgaagccggactacagtttggtcaagcaaacaatcttgtcatggattcagtcatataaataagggtcacccagcccaggggaggtgccccaccccatctgttccctctccctccccacactgagtccgaacctttcaggctttgcccccttcacacactccaaatttattcttctacttctcttcctgcaggtaccagactccggccacccagcgcttacctgggactgctgggcctctgccccgtggggacctgtcctccaggaaacaaggccagacacaggagggcagggaggactcttctccagggccggtgcgccgtctcttcagccccatggcacttgacttaggcagagcctacagcaccctcaccccagtccctgcagccaccaggaggtggtccccctcatcccattagccatcaccgccattcacagaggtctcggactgaggctggcagggggagcaccatgacccaagatcagaaccctgttgtctgtgcctctggagaggtgggggcaggagctgagggagggtttgggtggagaggggagaagatgcagtagcaggagcagatgctggcaggtagagacaaacttttatgacctttgccttctgacctttgcctctggccactgctccaactaaaacagaatggccccctctgggaacagggcttcctatgggctgggaagcatggagcccccacagtgtggctatgcaggggagtgaggaccaggtgggggcaggcctgtgggggtcacagagctgggctaagcttcagagggaagtggcccctgggagggggaatggctggggttaagaccctgggttcccacgcccccaaaacagaagtagaattagggagaaaggacccccaagaccaaggacggcacctatcagaggagctctccacgggcaggaggtgtcccagggtgagggtggccaggacaggtctagggaaatgcaggtggagcaggacccagagatggattggagatgccggaggggaggcttcctagcgggagcggagacaggcactgcagacaagtgtcagcgggaggggctctgggtggggaagaagactgggacttggaggaagacctctccagggagaagggaggagggggaaggagaaggggaggaagtgggaggaggagagtgctcatcctggaagccacagcctcggagagaactttctagaaggaaggcatgaccatcagtgtcccaggatgctgagaggccaggaaaggtgaggcctcaaggcgccatggggttgtggtgaccccaagatcactgggcacagagcagggatggctgggggtggaaggggaggggcgcgggctgaggttctagggccccaaagccaggtgtgatgtggctctagggggagtgaggaaggggagaatgtccctctgagcgtgcctcttggggaaggcaggggttctggctggggcttctccactcccaggaaaggaggtggtgtggaaggagcgggtgggacggaggagagagcgccccgcggccgcaggaccagcaggtgggggaccagggtcagcgctgctggaggggccttagcgcgacaggactggccagagaccggggatgtggcacagaaagagttaaagggcaccccagggaccgccctgccggtccacccatgtcacccatgttggcccctactccagcccccgtctgctctgcaggggaaggaaccgggagccgcggtgggggcgactgggggtgtcggtctttccagaaaatcaggcaggcatcaggaaagaaggggcgagaacccggggacgcgagaggaagggggcgagggggnnnnnnnnnnnnnnnnnnnnnnnnncgcgtcccagggcgtcccctcccgggactgggacccaccgcgaccaccacctgctgggccagggtccgcgggctcaggggtctgcaggattagggtctgcggaaccaggacccgtgggacaaaggtctgtggggcgcgggtccgcggggtggaattcagcgcgccgagtctgcgtatggccggggtacgaggcgctccctgcgcagggtgggcaggaccgaagctcgccgggagctgcgcggagggcgggcggggaccctccggtgccgctcccaccccgcggggccgcccccgagcccgccctccgccgccgccctcgccctcgtcgccgccggaaagtttgcaccgaccccgatctggcagcgccgcgaagacgagcggtcaccggcgcccgacccgagcgcgcccagaggacggcggggagccaagccgacccccgagcagcgccgcgcggtgagcacctgggccgcggacccgaggggacgttggggagtcgacccggtggggacagagaccgcggggcgggcgcggcggggccgggggcgcggggagcggggagccggccgggcggtctccggggtccgggctggtgcgctcctcagtcccgtcagacacccccgttcccaaccccggctcggacaccacccggtcctgcaccgtcgggcaggtccaggggtctcagcccctcccccgttctctggtcctggggggcgcggctgggggcgggggtgtcgctggccgcctggcgccctgcggcggccacactgcagcggccacactccccactcagggccccgggccccgccgccctggggagcgcacaaagcgccgcggacgcgtccccgaggcgcggggtctcaccagcgctgtctcccctcggtgggctcctgccccgaggactgcccggtggcaccggcgcggcccaggatggggtgaggggtgtctgcgccccgcctggccgctcctcttccgcggcccacactggcgactttgaccccggcaagcgggtcactgccctgcccggctccggcccccccggcgccccaccacccggccgactcggccaccgggcttatgctccgactctgaaccgactgaccccggccccctcggcgcccgcatcctccaaggaccggccagggctgctctctgcccttggtattggggacatcagggttggggggtctgggtgcacccacgcctgccccgcccccacggggtgagggcgcagggatagggctttgtcaacagcctgtggcccctgatcccgccccggtgccctgaccttccactaccttctctggtttcacaaaaacatcccggctcccatcccggagctcctcaaagcgtctgagaggccccttgcggacgccctgggagccccgctgccttcctggaccagtggccgctccacccatcctgggggcccagctccaggtctgcgggtccctcagccgcccccagtgggaatcggtggagcctgacgcagccaggagcgcccaagagtcacgtgttctgccagggaggacatgggacaggacacggggtgccagccctgcaaagcggccggggcagtggagctcaggtggccctaagccctggtggtggctggtgtggcccggcaggcagctgtgggagggaggaagggggtggcatgcggtgggggtctagagaaggcgggcagggcacctcgggagcccccccattgggcacctcgggaaccccccacattgggcacctcgggaaccctcccattgggcacctcgggaaccccccacattgggcacctcgggaacccccgcattgggcacctcgggaaccctcccattgggcacctcgggaacccccctattgggcacctcgggaacccccacattgggcacctcgggaaccccccctattgggcaccttgggaacccctcccctaattctcagctgactccaaggcctgagaaggagcttggtcacctggactgtgaaggtggagggtggggtccctggtgggtcgtcccacctaccagctgtgtcgccggaagggtaatacggagcactgtggccccggggagccccgagtggcagctccacagctgggagtttctgtccactccttcagtcaacaaacattgatcctgggctgaccggggcccgggggtgtcagtgtctcctctcgggggagagggctgggtgagatcaacagaggagcctcccttcttcccttcaggctggtgtcaccttcagtgatggggcagggtccccacttgggaagttaaatcgtcgtccccgtcccaggaccacagcagcctcagccctgctctccaggccaggctctctcatgggtgctcagctggaaattggtccccccccggctccacccacccctgttggggtgaggagctggagtctccctacccatatgggacccaccacccgcagggaacggaggacgctcacacttctgcacctcctgcctcactatcagagacccagtggagaattgcctcccacctcacctcttgtattcagaggccctgacccctagggatccgggactaggggtgccctatggggagcccacctgtggcctgtggatgctgagctgtcgggggaatcctccaggatccccagccccaccttcccaaccttctgttgaggctgaggggacacagagccccactcctgggtcctgactgtttcaaagaaaggcctgggggactgggcagccaacccctccctcggctcgctggggtctccagactggctgcccggctggaaggtggggccctggcacgcgaggacctcatgtgtggaggcactggcttggggggtgctcccagtggctctagagtcaacatgacaggcatcgaatggctcctgtttctctggcagagttggggcagagccaggcttggccacgctgggctctaaggggctgtcattttgcccagggagctcctggctgggtggtcctccccccagggtgagcacgcgtcccccccacccccacttcgaggcgcccaggcagggaacagctcattggccagtgtccttcctccttgtcccccgcctgcatctccaccatccaccctgctccagctgccccttgtccctctccccgtcccctgcccagagccccaggtctcccctgcacccctgagcctgcccacctagcagtgcccctcgtccagggcccctctgggttgggggtgcacacagtggggagaggcggctcctgctgctcctcacccagcccggctcagtggccggagccgcccaggacagtggcagtagatggggctgtttgatcaggatcagggaagataaggccccttgcgtgaccccagagctggggacgccaaaactgcccctcctcccccacccgcctgccgctgtctccgccagggagaggcccctactctgtgggtccttcgccccagcaccaagcctgcatggctgctcacctggctcaggaactggggatcagcgacacacgggtcctgcctcccatcggcccctacatgagcccagggtccaagggctgcggttgggagctctttagcagtctgtgacgcaggtgcctgtccctgtcattcagctgtcacactgcttggggcatctcaggccccgttagcggggcagccctgggtggagctggccccacgcgggctcacccagccgctacctggaggaggctaaaatccaggctgtcccgtggcagccagcagtccaggcctgcccggaaaccctctgctccagctgcagccttcgcccatctccttgcccctctccccggcttccccctggcactgccttccagctggctggccctccatctgcccagccatccatccacacctcttattccatttgagggtgccccaaagaagagcccgtaacagcccgggggctcatagccagccactcgcgggaccccgcacatgcacgtggacccacaggaagaccctccctgcttctcccacagaattcagttggtgcagaaactgggctctgtagcaacgaaaggccgatttgtgtagctgttgccaccccgaactcccagctcagatgctggctgtggcatggggaccaggggctgtgactcccacagccctggcaggcaccacgggggatgtcctccccaccctgtgcccccaccctaggccagctcctcctccaagtcgacgcccgcagtgctaacctcaaaggactgtgcagccagcctgtggcgtcccatgggatccaggaagcccaaccgagccttgcacggcacccacgaggcacctaggcaccccggtgctgggcagggggcacacatgtgacacagacccctgagtgtgggccccacacacttggcctggcacagctgcaagccagcccagccactttgctcgctgtggcactggggccaagtgatggaaggtccaggcaccgccaccctcacgcttggcacattggctcaggtcagcctggcaagccagctttcccaggggctaagaataggtgaggaggatggtgaggaagcagccgggggctgtcaactgagggaggaggtcaccatctggggaggctggtcccccacccaagagcattgggtcaccctgcaggaaggtggctgccaccagcaatgagacgaggggctctgcgaccctcagagctgccagccagccagccctgggtggcaagagtgactcctcctggggtctcctccctcctatcgccctctttttttttttttttttttttgagacggagtctcgctctgtcacccaggctggactgcaatggcgcaatctccgctcactgcaagctctgcctcccgggttcacatcattctcctgcctcaagctcccgagtagctgggactacaggcgcctgccaccacgcctggctaattttttgtatttttagtagacatggggtttcactgtgttagccaggatggtctcaatctccagacctcgtgatccacccccctcggcctcccaaagtgctgggattacaggtgtgagccaccacgcccagcccccagctccctctttatccctaggaccctgaggctcagaggggcagcttcaggggaggacaccccactggccaggacgccccaggctctgctgctctgccactcagctgccctcggaggagcgtacacacccaccaggactgcattgccccagctgtgcagcccctgccagatgtgggaggcagctagctgcccagaggcatgcccccctgccagccacagcgacccctgctgctgttgctgctgctgctggcctgccaggtgaggactcacagcaccctcagcacccaggggccctcctgtgaggactgcacactgatggctctctgtctgcctgcctgcctgcctgcctgtctgcctgcctgtctgtctgtctgcccgtctgcctgcccatctgcctgtctgtctgcctgtccgtctgtctgtccatctgtccatctgcctatccatctgcctgcctgtctgcctgtccgtctgcctgtctgtctgcctgtccatctgtccatctgcctatccatctgcctgcctgtctgtcggcctgcctgcctgcctgtctgtctgctgcctgtctgtccgtctgcctgtctgcctgtccgtctgcctgcctgtccgtctgcctgtccgtctgcctgcctgcctgtctgtctgcctgcctgtctgcctgcctgtccgtctgcctgtccgtctgcctgcctgtctgcctgcctgtctgcctgtctgcccgtctgcctgtctgtctgcctgtccgtctgcctgtctgtccgtctgtccatctgcctatccatctgcctgcctatctgtctgtccgtctgcctgcctgtctgtctgcctgtctgcctgtctgtctgcctgtctgtccatctgcctatccatctacctgcctgcctgtctgcctgtctgtctgcctgtctgtctgcctgcctgtctgtctgtctgtctggttgcttgtgcatgtgtcccccagccacaggtcccctccgctcaggtgatggacttcctgtttgagaagtggaagctctacggtgaccagtgtcaccacaacctgagcctgctgccccctcccacgggtgagccccccacccagagcctttcagcctgtgcctggcctcagcacttcctgagttctcttcatgggaaggttcctgggtgcttatgcagcctttgaggaccccgccaaggggccctgtcattcctcaggcccccaccaccgtgggcaggtgaggtaacgaggtaactgagccacagagctggggacttgcctcaggccgcagagccaggaaataacagaacggtggcattgccccagaaccggctgctgctgctgcccccaggcccagatgggtaataccacctacagccccgtggagttttcagtgggcagacagtgccagggcgtggaagctgggacccaggggcctgggagggctcgggtggagagtgtatatcatggcctggacacttggggtgcagggagaggatagggctggaggactcacccgggaggcagtgcctgggttcggatgagggaggcagccaccactgggcagaggggggcaggtgtggcagcctccattgggcagagggagcagatgtggcagccacaggtttggcgatgcacctgggaaggatgaaaatggcattggggttcagcccccagagagggaggtgctgagagaaggtcacggagaatgggggaccccagtgtgggtttggggcacatttgagatggggggtctccaagggaaggtgtcctgcagagctgcaattcagggctgggctgggcgtgctagcggaggctggtccaggggaggtggatggtcaggtgaggaaggtggaggtcagatgggggaggtggaggtcaagtgggggagggagcagcccaggccatgtcctgggcgaggtgacggccgagctcaggcttccagagagaggagagaggcctgctgagggagccccttctcccaccctgccctgccctgctctgccctgccctaccctaccctgcagagctggtgtgcaacagaaccttcgacaagtattcctgctggccggacacccccgccaataccacggccaacatctcctgcccctggtacctgccttggcaccacaaaggtacccatagaggggaggaactgtggggggggcgggcccagggtggggctgaccccagcctccccccacacccccagtgcaacaccgcttcgtgttcaagagatgcgggcccgacggtcagtgggtgcgtggaccccgggggcagccttggcgtgatgcctcccagtgccagatggatggcgaggagattgaggtccaggtcagtgggcggcaggcaggcgcggtggggctggatgggaacgggcatgggggcccctgcctggccctcacaggccactgtaactcgcagaaggaggtggccaagatgtacagcagcttccaggtgatgtacacagtgggctacagcctgtccctgggggccctgctcctcgccttggccatcctggggggcctcaggtaggattccgccagcgcccggggcggccgcagaggacagggaggaggacgggcgctgactggctgtgcccacagcaagctgcactgcacccgcaatgccatccacgcgaatctgtttgcgtccttcgtgctgaaagccagctccgtgctggtcattgatgggctgctcaggacccgctacagccagaaaattggcgacgacctcagtgtcagcacctggctcagtgatggagtgagcccccctcggcggccccaggcaggtgggtgggtgggcagccaggcaggtggccacgtagccgcgctcacactgcacctgtaccaggcggtggctggctgccgtgtggccgcggtgttcatgcaatatggcatcgtggccaactactgctggctgctggtggagggcctgtacctgcacaacctgctgggcctggccaccctccccgagaggagcttcttcagcctctacctgggcatcggctggggtgagtgggctggcatgagagggggttaaggcaggctgaccaagcctttgggaccacagctgctgccccccacaggtgcccccatgctgttcgtcgtcccctgggcagtggtcaagtgtctgttcgagaacgtccagtgagtatgagcggctggacagcctggggagggaccggggggctggggtgcggcgctctggcctgaggcagggaggggccggggatgagcctggtgcctggggagggggtcatttgtgaccttctcccttccttttctgagacccgaattagatcctggcaaaatcgggacgggggtgctgaggggcggaggggctgggggctgtgccccagtatgtgagtggcctggcctcgcaggtgctggaccagcaatgacaacatgggcttctggtggatcctgcggttccccgtcttcctggccatcctggtgaggaaatgaagagccaggagcgcaccccaggcccctcctcccttggcgtcctgaggctgccccaggagacagcagcatcctgtctgagagcgctgggagggagccggcacccagacaggacaccaggacactggccagcaccctggacactgagccaggctgttcctccctggctgtgtgcccaccagccccagggctatgtggcccagggcctatcttgctgccaggcccacctgcaggagggtcaggtggggccttccaagggcacagagctgttccctggggctcgggatgcccctgactcgcacccttctcacacagatcaacttcttcatcttcgtccgcatcgttcagctgctcgtggccaagctgcgggcacggcagatgcaccacacagactacaagttccggtgggtgccgcggcagctggcgtctcgagacctggagaccctcagggccagagggcagctgggggtggggactccaagctccacgtggatggtgcgggccgagggtgggggcggtgggtgactcaggcgctgcctctgcaggctggccaagtccacgctgaccctcatccctctgctgggcgtccacgaagtggtcttcgccttcgtgacggacgagcacgcccagggcaccctgcgctccgccaagctcttcttcgacctcttcctcagctccttccaggtgcccgcccgcccgccggctcccccgcccggggcgcagtgtgccacccctgaccaccctgtctctccagggcctgctggtggctgtcctctactgcttcctcaacaaggaggtaggtgggagtgggggcatctgagaccatcagcactggccgtcggggtcaggggcagagagaggcacagggatgccagccccacccctgcccgggggttggaacacgtggggcccaagcctttccctccccctgctcttattgggtgcagttgccatggcgctgggtgtcaggcccccaggacaggttggcctcagccccatcgctacggtgtccaccgtgggggtccccaggtgtctgcagactgctttccgtggcgatgctgggtggcatagctgtgcccagcagggagcttgtgtcgctctgcacccctcagagcggagactgggcatctccgatgaggcccacagcaggtcccggtggggtggagaggacaggcaggccctaggactggcctgccccgtccccctccccaggtgcagtcggagctgcggcggcgttggcaccgctggcgcctgggcaaagtgctatgggaggagcggaacaccagcaaccacagggcctcatcttcgcccggccacggccctcccagcaaggagctgcagtttgggaggggtggtggcagccaggattcatctgcggagacccccttggctggtggcctccctagattggctgagagccccttctgaaccctgctgggaccccagctagggctggactctggcacccagagggcgtcgctggacaacccagaactggacgcccagctgaggctgggggcgggggagccaacagcagcccccacctaccccccacccccagtgtggctgtctgcgagattgggcctcctctccctgcacctgccttgtccctggtgcagaggtgagcagaggagtccagggcgggagtgggggctgtgccgtgaactgcgtgccagtgtccccacgtatgtcggcacgtcccatgtgcatggaaatgtcctccaacaataaagagctcaagtggtcaccgtgcatgtcctggaaagcagggctggaaatgctggggccgaagcagtgggggatggaacagcggtgggtggtcagcgccagtgcgggctgttgaagggtccccctgctgtcccagttcactcagagttggcactggaaccccggaggatcccgaaggcagccagcctgtgcccatctgagcaggtcctggccaccttcccatcctggttctggcgggcagtccccctggacgctttggccaccagagggtcaccattcaccagcagagacgtgaggggcacagtggctaaggcggcatgaggcatcacagtcccctgaccgaccccatcagcactggattcacccgagggcgtcttctccctggaggccgtgaggacactggcacctggctcatcggcccgcccttcctctgagcctcctggcctccgtttcatctcagctccagccccctcgggcaatttacaggccacgtagcagattgaagcgggaagaaatgggcctgaacattgccgcgggtccaggcgacggaggagggcaggttgcccaacttctgcacaggacccggggtgcgccacacacacgccagtcctcgtgccacacagagaggtccggcctacgccagtcctcgtgccacacagagaggtccggcctacgccagtcctcgtgccacacagagaggtccggcctacgccagtcctcgtgccacacagagaggtccggcctacgccagtcctcgtgccacacagagaggtccggcctacgccagtcctcgtgccacacagagaggtccggcctacgccagtcctcttgccacctcgtggtgggtgggcgccctgcttgccagccagggagcaccaggaaagagctgcctcctgcgtgctggacacaggaggtgcttcagggtggggtctcccattgtgtggggcccaacctgagtctaagggcccagggaccacacagcgggggtggagacaaattcagggtagaagctgtgaggggcctgtggtcagccccccggggggtccctgcagcaggcactgtgagacctactgaggtgtgtgcatgggctggggaaggagccagtcaggtgcccctgctctgaggagctgctgggaagtgctgctgggccctgggggaaggggtgctcacagcccctgcctgggccacgtgggctggagccgctcaggcagagccggactaattggggcaaatgaggggacaggaggcctctgaggaaaggtaaatagaattactcacccgccaggcactggggccctcctgggggggccctcaccctgccacccaccacagggcctgcatgcagcagggagggaagtgagctgattaggcaaggctggacccttctggggccctggggttgctgtgattgggacggcaaggccaggagacggtcccctgagctgcacctgctggaggcctgtgatctcagaccttaaggcttcaggccagctctacgcccctccggcctcaggtcctggctctcctctgagccctggatgcccgggtgcctgtgtgggcacgaggctgctccgagtcagcacacggaggtggacattctccttcatgccagctgagctcagggctggtgactgccctggggaaactgcccctcacctgggacctcctgacagccctccccattcccgagtccctctgcccttgtcctctttcacctctgtcccgccctcatccctaagggaactggagcaggctggtggagttgggtggagttggggactggcagggggtggactcacccaggcaataaacactggccctaaccaggcagtcctgcaggcaggtaggtggagggactgttttttttcttttttggagatagagtctcactctgttgcccaagttggagtgcagtggcatgatcttggctcactgcaaactccacctcccaggttcatgtgattctctgcctcagcctcccgagtagctgggattataggcgtgtgccacgacacctggctaatttttttttttttttttttgagacggagtttcactctcgttgcccaggctggagctcaatggcgcgatctcagctcaccgcaacctccgcctcccaggttcaagcgattctcctgccttagcctccctagtagctgggattacaggcaggtatgtgatgcccggcatcccaaaggggtatctgcaagagttgggtgctgtgtgtgcatggctgggaggaagatgactttgataccctggaatctggtgtctgtggacacaaaaatactactaaaatgagagtggagaccaggaaaaaggaagacatgaactacatgaaggaccaaatctaggagagtcagaagtgcgtcacaggaataggggaccttgagccagacagaaggctcagcagagacaccctcaaggggatgaaagggattgagtgcactaatatttagaggagagagttcaggacttgattagtgactagtacatagaaaactaaacaaatgaggctgggtgcagtggctcatgcctgtaatcccagcactttggggggccaaggcgggcgaatcacctgaggtcaggagttcgagaccagcctggccaacatggtgaaacctcgtctctactgaaaatacaaaaattagccgggcgtggtggcgggcgcctgtagtcccagctacttgggagcctgaggcaggagaatcgcttgaacctgggaggcggaggctgctgtgagccaagatggtgccattgcactccaccctgggtgacagagcaagactccgtctcaaaaaaaaaaaaaaagaaagaaaaaaccaagcaaatgaaaaaagaaggcaattaataattccaaagaaaagaaaaatttgggcagaaaagaacaaaacaagcagaatttaccatgactcagttctgaatacaaacacagacatcataatgtaaacaccaacactgatgcaaccagaatcatgggagaaaaaagatctagggagggtggtggacgggaatatcacgtatgtactgggggtaggggagagaacaaaatgggaaaaatcaagaataattcacgttagaaataaaaatacagagcaaaatttaaaaatgcaaagaatgaggtgaagagttcaaagtggtcacctcggggccgggcgcggtggctcacgcctgtgatcccagcactctgggaggctgaggcgggcggatcacaaggccaggagtttgagaccatcctggctaacaaggagaaaccccatctctactaaaaattagccaggcgtggtggtgggcgcctgtagtcccagctactcgggaggctgaggcaggagaatggcgtgaacccaggaggcgcagcttgcagtgagccgagatcgcgccactgcactccagcttgggcaacagagtgagactccgcctcaaacaaaacaaaacaaaacaaaaaaacaaagtggtcatctctaggcaaggtgggtgggagatggctagggctgcaggtccactacgtgagctggctcagcctatccccagacaccctgcactcactcagcccggggtcctccccctgcactcactcagccccgggtcctcccctgcactcactcagccccgggtcctccccctgcactcactcagccccgggtcctcccctgcctgctctttctctgaccctgccctccactgttcctttttcttctttctctccctgttgtgtccaggaaccaggcaccaccctcatttcttcttgatcaatctttaaaaaccagcagtgctcagctaactcttcatctatctcccccgacctggggctctgctgaatccacgctttagacccagctatcagctcggcatgtacagctggatgtccacaccgagctgctcaccctgtccccagcttcttcctcccactgtccactgcagaagcctcctaacaggacccctgctgctaccccggaccctgcaacccattcccacacagcagccagatgctttgacacccgaagtctcctatgaatccgatgaggcctctgcaccacacctcattttacagaagtacaggggaaacaggggtctgttgacaccacagagatgcagctggccaaaggcagaatgtggggtacacgactgtcaaacgccaggggtccttacacgaatggtggaaaaagaggggcatgttacggatggaggctcgggacacatgggcgccgccttcccatgctgccagcaacccaccaggaacctatta

SEQ ID NO: 12 - DNA, 3182 bp, Hepatitis B virus
  source 1..3182: mol_type = other DNA
aattccacaacctttcaccaaactctgcaagatcccagagtgagaggcctgtatttccctgctggtggctccagttcaggagcagtaaaccctgttccgactactgcctctcccttatcgtcaatcttctcgaggattggggaccctgcgctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcctgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaaccaccagcacgggaccatgccgaacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctgaattttatgggttatgtcattggaagttatgggtccttgccacaagaacacatcatacaaaaaatcaaagaatgttttagaaaacttcctattaacaggcctattgattggaaagtatgtcaacgaattgtgggtcttttgggttttgctgccccatttacacaatgtggttatcctgcgttaatgcccttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcgtgcgtggaaccttttcggctcctctgccgatccatactgcggaactcctagccgcttgttttgctcgcagcaggtctggagcaaacattatcgggactgataactctgttgtcctctcccgcaaatatacatcgtatccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccgaatgttgcccaaggtcttacataagaggactcttggactctctgcaatgtcaacgaccgaccttgaggcatacttcaaagactgtttgtttaaagactgggaggagttgggggaggagattagattaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatcttctagataccgcctcagctctgtatcgggaagccttagagtctcctgagcattgttcacctcaccatactgcactcaggcaagcaattctttgctggggggaactaatgactctagctacctgggtgggtgttaatttggaagatccagcatctagagacctagtagtcagttatgtcaacactaatatgggcctaaagttcaggcaactcttgtggtttcacatttcttgtctcacttttggaagagaaaccgttatagagtatttggtgtctttcggagtgtggattcgcactcctccagcttatagaccaccaaatgcccctatcctatcaacacttccggaaactactgttgttagacgacgaggcaggtcccctagaagaagaactccctcgcctcgcagacgaaggtctcaatcgccgcgtcgcagaagatctcaatctcgggaacctcaatgttagtattccttggactcataaggtggggaactttactggtctttattcttctactgtacctgtctttaatcctcattggaaaacaccatcttttcctaatatacatttacaccaagacattatcaaaaaatgtgaacagtttgtaggcccacttacagttaatgagaaaagaagattgcaattgattatgcctgctaggttttatccaaaggttaccaaatatttaccattggataagggtattaaaccttattatccagaacatctagttaatcattacttccaaactagacactatttacacactctatggaaggcgggtatattatataagagagaaacaacacatagcgcctcattttgtgggtcaccatattcttgggaacaagatctacagcatggggcagaatctttccaccagcaatcctctgggattctttcccgaccaccagttggatccagccttcagagcaaacacagcaaatccagattgggacttcaatcccaacaaggacacctggccagacgccaacaaggtaggagctggagcattcgggctgggtttcaccccaccgcacggaggccttttggggtggagccctcaggctcagggcatactacaaactttgccagcaaatccgcctcctgcctccaccaatcgccagacaggaaggcagcctaccccgctgtctccacctttgagaaacactcatcctcaggccatgcagtgg

SEQ ID NO: 13 - RNA, 3318 nt, Homo sapiens
  source 1..3318: mol_type = mRNA
gtgatgcgtagttccggctgccggttgacatgaagaagcagcagcggctagggcggcggtagctgcaggggtcggggattgcagcgggcctcggggctaagagcgcgacgcggcctagagcggcagacggcgcagtgggccgagaaggaggcgcagcagccgccctggcccgtcatggagatggaaaaggagttcgagcagatcgacaagtccgggagctgggcggccatttaccaggatatccgacatgaagccagtgacttcccatgtagagtggccaagcttcctaagaacaaaaaccgaaataggtacagagacgtcagtccctttgaccatagtcggattaaactacatcaagaagataatgactatatcaacgctagtttgataaaaatggaagaagcccaaaggagttacattcttacccagggccctttgcctaacacatgcggtcacttttgggagatggtgtgggagcagaaaagcaggggtgtcgtcatgctcaacagagtgatggagaaaggttcgttaaaatgcgcacaatactggccacaaaaagaagaaaaagagatgatctttgaagacacaaatttgaaattaacattgatctctgaagatatcaagtcatattatacagtgcgacagctagaattggaaaaccttacaacccaagaaactcgagagatcttacatttccactataccacatggcctgactttggagtccctgaatcaccagcctcattcttgaactttcttttcaaagtccgagagtcagggtcactcagcccggagcacgggcccgttgtggtgcactgcagtgcaggcatcggcaggtctggaaccttctgtctggctgatacctgcctcttgctgatggacaagaggaaagacccttcttccgttgatatcaagaaagtgctgttagaaatgaggaagtttcggatggggctgatccagacagccgaccagctgcgcttctcctacctggctgtgatcgaaggtgccaaattcatcatgggggactcttccgtgcaggatcagtggaaggagctttcccacgaggacctggagcccccacccgagcatatccccccacctccccggccacccaaacgaatcctggagccacacaatgggaaatgcagggagttcttcccaaatcaccagtgggtgaaggaagagacccaggaggataaagactgccccatcaaggaagaaaaaggaagccccttaaatgccgcaccctacggcatcgaaagcatgagtcaagacactgaagttagaagtcgggtcgtggggggaagtcttcgaggtgcccaggctgcctccccagccaaaggggagccgtcactgcccgagaaggacgaggaccatgcactgagttactggaagcccttcctggtcaacatgtgcgtggctacggtcctcacggccggcgcttacctctgctacaggttcctgttcaacagcaacacatagcctgaccctcctccactccacctccacccactgtccgcctctgcccgcagagcccacgcccgactagcaggcatgccgcggtaggtaagggccgccggaccgcgtagagagccgggccccggacggacgttggttctgcactaaaacccatcttccccggatgtgtgtctcacccctcatccttttactttttgccccttccactttgagtaccaaatccacaagccattttttgaggagagtgaaagagagtaccatgctggcggcgcagagggaaggggcctacacccgtcttggggctcgccccacccagggctccctcctggagcatcccaggcgggcggcacgccaacagccccccccttgaatctgcagggagcaactctccactccatatttatttaaacaattttttccccaaaggcatccatagtgcactagcattttcttgaaccaataatgtattaaaattttttgatgtcagccttgcatcaagggctttatcaaaaagtacaataataaatcctcaggtagtactgggaatggaaggctttgccatgggcctgctgcgtcagaccagtactgggaaggaggacggttgtaagcagttgttatttagtgatattgtgggtaacgtgagaagatagaacaatgctataatatataatgaacacgtgggtatttaataagaaacatgatgtgagattactttgtcccgcttattctcctccctgttatctgctagatctagttctcaatcactgctcccccgtgtgtattagaatgcatgtaaggtcttcttgtgtcctgatgaaaaatatgtgcttgaaatgagaaactttgatctctgcttactaatgtgccccatgtccaagtccaacctgcctgtgcatgacctgatcattacatggctgtggttcctaagcctgttgctgaagtcattgtcgctcagcaatagggtgcagttttccaggaataggcatttgcctaattcctggcatgacactctagtgacttcctggtgaggcccagcctgtcctggtacagcagggtcttgctgtaactcagacattccaagggtatgggaagccatattcacacctcacgctctggacatgatttagggaagcagggacaccccccgccccccacctttgggatcagcctccgccattccaagtcaacactcttcttgagcagaccgtgatttggaagagaggcacctgctggaaaccacacttcttgaaacagcctgggtgacggtcctttaggcagcctgccgccgtctctgtcccggttcaccttgccgagagaggcgcgtctgccccaccctcaaaccctgtggggcctgatggtgctcacgactcttcctgcaaagggaactgaagacctccacattaagtggctttttaacatgaaaaacacggcagctgtagctcccgagctactctcttgccagcattttcacattttgcctttctcgtggtagaagccagtacagagaaattctgtggtgggaacattcgaggtgtcaccctgcagagctatggtgaggtgtggataaggcttaggtgccaggctgtaagcattctgagctgggcttgttgtttttaagtcctgtatatgtatgtagtagtttgggtgtgtatatatagtagcatttcaaaatggacgtactggtttaacctcctatccttggagagcagctggctctccaccttgttacacattatgttagagaggtagcgagctgctctgctatatgccttaagccaatatttactcatcaggtcattattttttacaatggccatggaataaaccatttttacaaaa

SEQ ID NO: 14 - DNA, 10 bp, Homo sapiens
  source 1..10: mol_type = genomic DNA
aaaaaaaaaa

SEQ ID NO: 15 - RNA, 4978 nt, Homo sapiens
  source 1..4978: mol_type = mRNA
ggtttccggagctgcggcggcgcagactgggagggggagccgggggttccgacgtcgcagccgagggaacaagccccaaccggatcctggacaggcaccccggcttggcgctgtctctccccctcggctcggagaggcccttcggcctgagggagcctcgccgcccgtccccggcacacgcgcagccccggcctctcggcctctgccggagaaacagttgggacccctgattttagcaggatggcccaatggaatcagctacagcagcttgacacacggtacctggagcagctccatcagctctacagtgacagcttcccaatggagctgcggcagtttctggccccttggattgagagtcaagattgggcatatgcggccagcaaagaatcacatgccactttggtgtttcataatctcctgggagagattgaccagcagtatagccgcttcctgcaagagtcgaatgttctctatcagcacaatctacgaagaatcaagcagtttcttcagagcaggtatcttgagaagccaatggagattgcccggattgtggcccggtgcctgtgggaagaatcacgccttctacagactgcagccactgcggcccagcaagggggccaggccaaccaccccacagcagccgtggtgacggagaagcagcagatgctggagcagcaccttcaggatgtccggaagagagtgcaggatctagaacagaaaatgaaagtggtagagaatctccaggatgactttgatttcaactataaaaccctcaagagtcaaggagacatgcaagatctgaatggaaacaaccagtcagtgaccaggcagaagatgcagcagctggaacagatgctcactgcgctggaccagatgcggagaagcatcgtgagtgagctggcggggcttttgtcagcgatggagtacgtgcagaaaactctcacggacgaggagctggctgactggaagaggcggcaacagattgcctgcattggaggcccgcccaacatctgcctagatcggctagaaaactggataacgtcattagcagaatctcaacttcagacccgtcaacaaattaagaaactggaggagttgcagcaaaaagtttcctacaaaggggaccccattgtacagcaccggccgatgctggaggagagaatcgtggagctgtttagaaacttaatgaaaagtgcctttgtggtggagcggcagccctgcatgcccatgcatcctgaccggcccctcgtcatcaagaccggcgtccagttcactactaaagtcaggttgctggtcaaattccctgagttgaattatcagcttaaaattaaagtgtgcattgacaaagactctggggacgttgcagctctcagaggatcccggaaatttaacattctgggcacaaacacaaaagtgatgaacatggaagaatccaacaacggcagcctctctgcagaattcaaacacttgaccctgagggagcagagatgtgggaatgggggccgagccaattgtgatgcttccctgattgtgactgaggagctgcacctgatcacctttgagaccgaggtgtatcaccaaggcctcaagattgacctagagacccactccttgccagttgtggtgatctccaacatctgtcagatgccaaatgcctgggcgtccatcctgtggtacaacatgctgaccaacaatcccaagaatgtaaacttttttaccaagcccccaattggaacctgggatcaagtggccgaggtcctgagctggcagttctcctccaccaccaagcgaggactgagcatcgagcagctgactacactggcagagaaactcttgggacctggtgtgaattattcagggtgtcagatcacatgggctaaattttgcaaagaaaacatggctggcaagggcttctccttctgggtctggctggacaatatcattgaccttgtgaaaaagtacatcctggccctttggaacgaagggtacatcatgggctttatcagtaaggagcgggagcgggccatcttgagcactaagcctccaggcaccttcctgctaagattcagtgaaagcagcaaagaaggaggcgtcactttcacttgggtggagaaggacatcagcggtaagacccagatccagtccgtggaaccatacacaaagcagcagctgaacaacatgtcatttgctgaaatcatcatgggctataagatcatggatgctaccaatatcctggtgtctccactggtctatctctatcctgacattcccaaggaggaggcattcggaaagtattgtcggccagagagccaggagcatcctgaagctgacccaggtagcgctgccccatacctgaagaccaagtttatctgtgtgacaccaacgacctgcagcaataccattgacctgccgatgtccccccgcactttagattcattgatgcagtttggaaataatggtgaaggtgctgaaccctcagcaggagggcagtttgagtccctcacctttgacatggagttgacctcggagtgcgctacctcccccatgtgaggagctgagaacggaagctgcagaaagatacgactgaggcgcctacctgcattctgccacccctcacacagccaaaccccagatcatctgaaactactaactttgtggttccagattttttttaatctcctacttctgctatctttgagcaatctgggcacttttaaaaatagagaaatgagtgaatgtgggtgatctgcttttatctaaatgcaaataaggatgtgttctctgagacccatgatcaggggatgtggcggggggtggctagagggagaaaaaggaaatgtcttgtgttgttttgttcccctgccctcctttctcagcagctttttgttattgttgttgttgttcttagacaagtgcctcctggtgcctgcggcatccttctgcctgtttctgtaagcaaatgccacaggccacctatagctacatactcctggcattgcactttttaaccttgctgacatccaaatagaagataggactatctaagccctaggtttctttttaaattaagaaataataacaattaaagggcaaaaaacactgtatcagcatagcctttctgtatttaagaaacttaagcagccgggcatggtggctcacgcctgtaatcccagcactttgggaggccgaggcggatcataaggtcaggagatcaagaccatcctggctaacacggtgaaaccccgtctctactaaaagtacaaaaaattagctgggtgtggtggtgggcgcctgtagtcccagctactcgggaggctgaggcaggagaatcgcttgaacctgagaggcggaggttgcagtgagccaaaattgcaccactgcacactgcactccatcctgggcgacagtctgagactctgtctcaaaaaaaaaaaaaaaaaaaagaaacttcagttaacagcctccttggtgctttaagcattcagcttccttcaggctggtaatttatataatccctgaaacgggcttcaggtcaaacccttaagacatctgaagctgcaacctggcctttggtgttgaaataggaaggtttaaggagaatctaagcattttagacttttttttataaatagacttattttcctttgtaatgtattggccttttagtgagtaaggctgggcagagggtgcttacaaccttgactccctttctccctggacttgatctgctgtttcagaggctaggttgtttctgtgggtgccttatcagggctgggatacttctgattctggcttccttcctgccccaccctcccgaccccagtccccctgatcctgctagaggcatgtctccttgcgtgtctaaaggtccctcatcctgtttgttttaggaatcctggtctcaggacctcatggaagaagagggggagagagttacaggttggacatgatgcacactatggggccccagcgacgtgtctggttgagctcagggaatatggttcttagccagtttcttggtgatatccagtggcacttgtaatggcgtcttcattcagttcatgcagggcaaaggcttactgataaacttgagtctgccctcgtatgagggtgtatacctggcctccctctgaggctggtgactcctccctgctggggccccacaggtgaggcagaacagctagagggcctccccgcctgcccgccttggctggctagctcgcctctcctgtgcgtatgggaacacctagcacgtgctggatgggctgcctctgactcagaggcatggccggatttggcaactcaaaaccaccttgcctcagctgatcagagtttctgtggaattctgtttgttaaatcaaattagctggtctctgaattaagggggagacgaccttctctaagatgaacagggttcgccccagtcctcctgcctggagacagttgatgtgtcatgcagagctcttacttctccagcaacactcttcagtacataataagcttaactgataaacagaatatttagaaaggtgagacttgggcttaccattgggtttaaatcatagggacctagggcgagggttcagggcttctctggagcagatattgtcaagttcatggccttaggtagcatgtatctggtcttaactctgattgtagcaaaagttctgagaggagctgagccctgttgtggcccattaaagaacagggtcctcaggccctgcccgcttcctgtccactgccccctccccatccccagcccagccgagggaatcccgtgggttgcttacctacctataaggtggtttataagctgctgtcctggccactgcattcaaattccaatgtgtacttcatagtgtaaaaatttatattattgtgaggttttttgtcttttttttttttttttttttttggtatattgctgtatctactttaacttccagaaataaacgttatataggaaccgtaaaaa

SEQ ID NO: 16 - RNA, 938 nt, Homo sapiens
  source 1..938: mol_type = mRNA
gttgactaagtcaataatcagaatcagcaggtttgcagtcagattggcagggataagcagcctagctcaggagaagtgagtataaaagccccaggctgggagcagccatcacagaagtccactcattcttggcaggatggcttctcatcgtctgctcctcctctgccttgctggactggtatttgtgtctgaggctggccctacgggcaccggtgaatccaagtgtcctctgatggtcaaagttctagatgctgtccgaggcagtcctgccatcaatgtggccgtgcatgtgttcagaaaggctgctgatgacacctgggagccatttgcctctgggaaaaccagtgagtctggagagctgcatgggctcacaactgaggaggaatttgtagaagggatatacaaagtggaaatagacaccaaatcttactggaaggcacttggcatctccccattccatgagcatgcagaggtggtattcacagccaacgactccggcccccgccgctacaccattgccgccctgctgagcccctactcctattccaccacggctgtcgtcaccaatcccaaggaatgagggacttctcctccagtggacctgaaggacgagggatgggatttcatgtaaccaagagtattccatttttactaaagcagtgttttcacctcatatgctatgttagaagtccaggcagagacaataaaacattcctgtgaaaggcacttttcattccactttaacttgattttttaaattcccttattgtcccttccaaaaaaaagagaatcaaaattttacaaagaatcaaaggaattctagaaagtatctgggcagaacgctaggagagatccaaatttccattgtcttgcaagcaaagcacgtattaaatatgatctgcagccattaaaaagacacattctgtaaaaaaaaaaaa

SEQ ID NO: 17 - DNA, 16 bp, Synthetic construct
  source 1..16: mol_type = other DNA
ttgatttaatggttgc

SEQ ID NO: 18 - DNA, 16 bp, Synthetic construct
  source 1..16: mol_type = other DNA
tgatttaatggttgca

SEQ ID NO: 19 - DNA, 16 bp, Synthetic construct
  source 1..16: mol_type = other DNA
gatttaatggttgcaa

SEQ ID NO: 20 - DNA, 16 bp, Synthetic construct
  source 1..16: mol_type = other DNA
agtcgcgactctggta

SEQ ID NO: 21 - DNA, 16 bp, Synthetic construct
  source 1..16: mol_type = other DNA
gtcaatatcaaagcac

SEQ ID NO: 22 - DNA, 16 bp, Synthetic construct
  source 1..16: mol_type = other DNA
gaacattattaggcta

SEQ ID NO: 23 - DNA, 16 bp, Synthetic construct
  source 1..16: mol_type = other DNA
ccttatggatgctgct

SEQ ID NO: 24 - DNA, 16 bp, Synthetic construct
  source 1..16: mol_type = other DNA
cattgtactatgccag

SEQ ID NO: 25 - DNA, 20 bp, Synthetic construct
  source 1..20: mol_type = other DNA
tgctccgttggtgcttgttc

SEQ ID NO: 26 - DNA, 20 bp, Synthetic construct
  source 1..20: mol_type = other DNA
tcttcctgtgacagtggtgg

SEQ ID NO: 27 - DNA, 20 bp, Synthetic construct
  source 1..20: mol_type = other DNA
ttcttcctgtgacagtggtg

SEQ ID NO: 28 - DNA, 20 bp, Synthetic construct
  source 1..20: mol_type = other DNA
ggttcttcctgtgacagtgg

SEQ ID NO: 29 - DNA, 20 bp, Synthetic construct
  source 1..20: mol_type = other DNA
cgactatgcgagtgtggtgt

SEQ ID NO: 30 - DNA, 20 bp, Synthetic construct
  source 1..20: mol_type = other DNA
ccgactatgcgagtgtggtg

SEQ ID NO: 31 - DNA, 20 bp, Synthetic construct
  source 1..20: mol_type = other DNA
gcctcagtctgcttcgcacc

SEQ ID NO: 32 - DNA, 20 bp, Synthetic construct
  source 1..20: mol_type = other DNA
agcttcttgtccagctttat

SEQ ID NO: 33 - DNA, 20 bp, Synthetic construct
  source 1..20: mol_type = other DNA
agcatagttaacgagctccc

SEQ ID NO: 34 - DNA, 20 bp, Synthetic construct
  source 1..20: mol_type = other DNA
tgtcatattcctggatcctt

SEQ ID NO: 35 - DNA, 16 bp, Synthetic construct
  source 1..16: mol_type = other DNA
ggacacccacgccccc

SEQ ID NO: 36 - DNA, 20 bp, Synthetic construct
  source 1..20: mol_type = other DNA
ccctcctgtgcctggatgct

SEQ ID NO: 37 - DNA, 16 bp, Synthetic construct
  source 1..16: mol_type = other DNA
gctaaacaaccgcctt

SEQ ID NO: 38 - DNA, 20 bp, Synthetic construct
  source 1..20: mol_type = other DNA
atgcatggtgatgcttctga

SEQ ID NO: 39 - DNA, 16 bp, Synthetic construct
  source 1..16: mol_type = other DNA
catggtgatgcttctg

SEQ ID NO: 40 - DNA, 16 bp, Synthetic construct
  source 1..16: mol_type = other DNA
agagctaaacaaccgc

SEQ ID NO: 41 - DNA, 16 bp, Synthetic construct
  source 1..16: mol_type = other DNA
actcccgggacaccca

SEQ ID NO: 42 - DNA, 16 bp, Synthetic construct
  source 1..16: mol_type = other DNA
acaccctcgcctccgg

SEQ ID NO: 43 - DNA, 16 bp, Synthetic construct
  source 1..16: mol_type = other DNA
gcctccggaacaccca

SEQ ID NO: 44 - DNA, 20 bp, Synthetic construct
  source 1..20: mol_type = other DNA
acggcattggtgcacagttt

SEQ ID NO: 45 - DNA, 20 bp, Synthetic construct
  source 1..20: mol_type = other DNA
gcaaccgggatgatgagtgc

SEQ ID NO: 46 - DNA, 20 bp, Synthetic construct
  source 1..20: mol_type = other DNA
tgcacagtttctggcaggcc

SEQ ID NO: 47 - DNA, 20 bp, Synthetic construct
  source 1..20: mol_type = other DNA
ggcagcggacggcattggtg

SEQ ID NO: 48 - DNA, 20 bp, Synthetic construct
  source 1..20: mol_type = other DNA
ggtgcacagtttctggcagg

SEQ ID NO: 49 - DNA, 20 bp, Synthetic construct
  source 1..20: mol_type = other DNA
gcagccatggtgatcaggag

SEQ ID NO: 50 - DNA, 20 bp, Synthetic construct
  source 1..20: mol_type = other DNA
ggtagaaatatagttgttcc

SEQ ID NO: 51 - DNA, 20 bp, Synthetic construct
  source 1..20: mol_type = other DNA
ttcatgtgtctgcatcatgt

SEQ ID NO: 52 - DNA, 20 bp, Synthetic construct
  source 1..20: mol_type = other DNA
gcatccagcgagcaccaaag

SEQ ID NO: 53 - DNA, 20 bp, Synthetic construct
  source 1..20: mol_type = other DNA
agccatggtgatcaggaggc

SEQ ID NO: 54 - DNA, 20 bp, Synthetic construct
  source 1..20: mol_type = other DNA
cagccatggtgatcaggagg

SEQ ID NO: 55 - DNA, 20 bp, Synthetic construct
  source 1..20: mol_type = other DNA
gtctggattacagcataaac

SEQ ID NO: 56 - DNA, 20 bp, Synthetic construct
  source 1..20: mol_type = other DNA
ggtctggattacagcataaa

SEQ ID NO: 57 - DNA, 20 bp, Synthetic construct
  source 1..20: mol_type = other DNA
ccttggtctggattacagca

SEQ ID NO: 58 - DNA, 20 bp, Synthetic construct
  source 1..20: mol_type = other DNA
ccttggtctggattacagca

SEQ ID NO: 59 - DNA, 20 bp, Synthetic construct
  source 1..20: mol_type = other DNA
gtgcttgtccaggatgatgc

SEQ ID NO: 60 - DNA, 17 bp, Synthetic construct
  source 1..17: mol_type = other DNA
ggttcccgaggtgccca

SEQ ID NO: 61 - DNA, 20 bp, Synthetic construct
  source 1..20: mol_type = other DNA
tccacaggccacaggtgggc

SEQ ID NO: 62 - DNA, 20 bp, Synthetic construct
  source 1..20: mol_type = other DNA
ctctttattgttggaggaca

SEQ ID NO: 63 - DNA, 20 bp, Synthetic construct
  source 1..20: mol_type = other DNA
gcaaggctcggttgggcttc

SEQ ID NO: 64 - DNA, 17 bp, Synthetic construct
  source 1..17: mol_type = other DNA
gggcaatgcagtcctgg

SEQ ID NO: 65 - DNA, 17 bp, Synthetic construct
  source 1..17: mol_type = other DNA
gaaggtgacaccagcct

SEQ ID NO: 66 - DNA, 17 bp, Synthetic construct
  source 1..17: mol_type = other DNA
gctcagcatccacaggc

SEQ ID NO: 67 - DNA, 21 bp, Synthetic construct
  source 1..21: mol_type = other DNA
gggttcccgaggtgcccaatg

SEQ ID NO: 68 - DNA, 20 bp, Synthetic construct
  source 1..20: mol_type = other DNA
gcagaggtgaagcgaagtgc

SEQ ID NO: 69 - DNA, 20 bp, Synthetic construct
  source 1..20: mol_type = other DNA
aatggtttattccatggcca

SEQ ID NO: 70 - DNA, 20 bp, Synthetic construct
  source 1..20: mol_type = other DNA
ggtttattccatggccattg

SEQ ID NO: 71 - DNA, 20 bp, Synthetic construct
  source 1..20: mol_type = other DNA
aaatggtttattccatggcc

SEQ ID NO: 72 - DNA, 18 bp, Synthetic construct
  source 1..18: mol_type = other DNA
aatggtttattccatggc

SEQ ID NO: 73 - DNA, 16 bp, Synthetic construct
  source 1..16: mol_type = other DNA
ctatttggatgtcagc

SEQ ID NO: 74 - DNA, 20 bp, Synthetic construct
  source 1..20: mol_type = other DNA
tcttggttacatgaaatccc

SEQ ID NO: 75 - DNA, 20 bp, Synthetic construct
  source 1..20: mol_type = other DNA
cttggttacatgaaatccca

SEQ ID NO: 76 - DNA, 20 bp, Synthetic construct
  source 1..20: mol_type = other DNA
ggaatactcttggttacatg

SEQ ID NO: 77 - DNA, 20 bp, Synthetic construct
  source 1..20: mol_type = other DNA
tggaatactcttggttacat

SEQ ID NO: 78 - DNA, 20 bp, Synthetic construct
  source 1..20: mol_type = other DNA
ttttattgtctctgcctgga

SEQ ID NO: 79 - DNA, 20 bp, Synthetic construct
  source 1..20: mol_type = other DNA
gaatgttttattgtctctgc

SEQ ID NO: 80 - DNA, 20 bp, Synthetic construct
  source 1..20: mol_type = other DNA
aggaatgttttattgtctct

SEQ ID NO: 81 - DNA, 20 bp, Synthetic construct
  source 1..20: mol_type = other DNA
acaggaatgttttattgtct

SEQ ID NO: 82 - RNA, 3731 nt, Homo sapiens
  source 1..3731: mol_type = mRNA
gtccgatggggctctggtggcgtgatctgcgcgccccaggcgtcaagcacccacaccctagaaggtttccgcagcgacgtcgaggcgctcatggttgcaggcgggcgccgccgttcagttcagggtctgagcctggaggagtgagccaggcagtgagactggctcgggcgggccgggacgcgtcgttgcagcagcggctcccagctcccagccaggattccgcgcgccccttcacgcgccctgctcctgaacttcagctcctgcacagtcctccccaccgcaaggctcaaggcgccgccggcgtggaccgcgcacggcctctaggtctcctcgccaggacagcaacctctcccctggccctcatgggcaccgtcagctccaggcggtcctggtggccgctgccactgctgctgctgctgctgctgctcctgggtcccgcgggcgcccgtgcgcaggaggacgaggacggcgactacgaggagctggtgctagccttgcgttccgaggaggacggcctggccgaagcacccgagcacggaaccacagccaccttccaccgctgcgccaaggatccgtggaggttgcctggcacctacgtggtggtgctgaaggaggagacccacctctcgcagtcagagcgcactgcccgccgcctgcaggcccaggctgcccgccggggatacctcaccaagatcctgcatgtcttccatggccttcttcctggcttcctggtgaagatgagtggcgacctgctggagctggccttgaagttgccccatgtcgactacatcgaggaggactcctctgtctttgcccagagcatcccgtggaacctggagcggattacccctccacggtaccgggcggatgaataccagccccccgacggaggcagcctggtggaggtgtatctcctagacaccagcatacagagtgaccaccgggaaatcgagggcagggtcatggtcaccgacttcgagaatgtgcccgaggaggacgggacccgcttccacagacaggccagcaagtgtgacagtcatggcacccacctggcaggggtggtcagcggccgggatgccggcgtggccaagggtgccagcatgcgcagcctgcgcgtgctcaactgccaagggaagggcacggttagcggcaccctcataggcctggagtttattcggaaaagccagctggtccagcctgtggggccactggtggtgctgctgcccctggcgggtgggtacagccgcgtcctcaacgccgcctgccagcgcctggcgagggctggggtcgtgctggtcaccgctgccggcaacttccgggacgatgcctgcctctactccccagcctcagctcccgaggtcatcacagttggggccaccaatgcccaagaccagccggtgaccctggggactttggggaccaactttggccgctgtgtggacctctttgccccaggggaggacatcattggtgcctccagcgactgcagcacctgctttgtgtcacagagtgggacatcacaggctgctgcccacgtggctggcattgcagccatgatgctgtctgccgagccggagctcaccctggccgagttgaggcagagactgatccacttctctgccaaagatgtcatcaatgaggcctggttccctgaggaccagcgggtactgacccccaacctggtggccgccctgccccccagcacccatggggcaggttggcagctgttttgcaggactgtatggtcagcacactcggggcctacacggatggccacagccgtcgcccgctgcgccccagatgaggagctgctgagctgctccagtttctccaggagtgggaagcggcggggcgagcgcatggaggcccaagggggcaagctggtctgccgggcccacaacgcttttgggggtgagggtgtctacgccattgccaggtgctgcctgctaccccaggccaactgcagcgtccacacagctccaccagctgaggccagcatggggacccgtgtccactgccaccaacagggccacgtcctcacaggctgcagctcccactgggaggtggaggaccttggcacccacaagccgcctgtgctgaggccacgaggtcagcccaaccagtgcgtgggccacagggaggccagcatccacgcttcctgctgccatgccccaggtctggaatgcaaagtcaaggagcatggaatcccggcccctcaggagcaggtgaccgtggcctgcgaggagggctggaccctgactggctgcagtgccctccctgggacctcccacgtcctgggggcctacgccgtagacaacacgtgtgtagtcaggagccgggacgtcagcactacaggcagcaccagcgaaggggccgtgacagccgttgccatctgctgccggagccggcacctggcgcaggcctcccaggagctccagtgacagccccatcccaggatgggtgtctggggagggtcaagggctggggctgagctttaaaatggttccgacttgtccctctctcagccctccatggcctggcacgaggggatggggatgcttccgcctttccggggctgctggcctggcccttgagtggggcagcctccttgcctggaactcactcactctgggtgcctcctccccaggtggaggtgccaggaagctccctccctcactgtggggcatttcaccattcaaacaggtcgagctgtgctcgggtgctgccagctgctcccaatgtgccgatgtccgtgggcagaatgacttttattgagctcttgttccgtgccaggcattcaatcctcaggtctccaccaaggaggcaggattcttcccatggataggggagggggcggtaggggctgcagggacaaacatcgttggggggtgagtgtgaaaggtgctgatggccctcatctccagctaactgtggagaagcccctgggggctccctgattaatggaggcttagctttctggatggcatctagccagaggctggagacaggtgcgcccctggtggtcacaggctgtgccttggtttcctgagccacctttactctgctctatgccaggctgtgctagcaacacccaaaggtggcctgcggggagccatcacctaggactgactcggcagtgtgcagtggtgcatgcactgtctcagccaacccgctccactacccggcagggtacacattcgcacccctacttcacagaggaagaaacctggaaccagagggggcgtgcctgccaagctcacacagcaggaactgagccagaaacgcagattgggctggctctgaagccaagcctcttcttacttcacccggctgggctcctcatttttacgggtaacagtgaggctgggaaggggaacacagaccaggaagctcggtgagtgatggcagaacgatgcctgcaggcatggaactttttccgttatcacccaggcctgattcactggcctggcggagatgcttctaaggcatggtcgggggagagggccaacaactgtccctccttgagcaccagccccacccaagcaagcagacatttatcttttgggtctgtcctctctgttgcctttttacagccaacttttctagacctgttttgcttttgtaacttgaagatatttattctgggttttgtagcatttttattaatatggtgactttttaaaataaaaacaaacaaacgttgtcctaacaaaaaaaaaaaaaaaaaaaaa

SEQ ID NO: 83 - DNA, 20 bp, Synthetic construct
  source 1..20: mol_type = other DNA
ccttggccacgccggcatcc

SEQ ID NO: 84 - DNA, 20 bp, Synthetic construct
  source 1..20: mol_type = other DNA
gtcacacttgctggcctgtc

SEQ ID NO: 85 - DNA, 20 bp, Synthetic construct
  source 1..20: mol_type = other DNA
tggcagtggacacgggtccc

SEQ ID NO: 86 - DNA, 20 bp, Synthetic construct
  source 1..20: mol_type = other DNA
actcaccgagcttcctggtc

SEQ ID NO: 87 - DNA, 20 bp, Synthetic construct
  source 1..20: mol_type = other DNA
atcccacgcccctgtccagc

SEQ ID NO: 88 - DNA, 20 bp, Synthetic construct
  source 1..20: mol_type = other DNA
ggacattgccagtaatcgca

SEQ ID NO: 89 - DNA, 20 bp, Synthetic construct
  source 1..20: mol_type = other DNA
tgcaagtctcttggcaaaca

SEQ ID NO: 90 - DNA, 20 bp, Synthetic construct
  source 1..20: mol_type = other DNA
agcatagattttgacttccc

SEQ ID NO: 91 - DNA, 20 bp, Synthetic construct
  source 1..20: mol_type = other DNA
ccacctttgggtgaatagca

SEQ ID NO: 92 - DNA, 20 bp, Synthetic construct
  source 1..20: mol_type = other DNA
actcaacttgaggacaataa

SEQ ID NO: 93 - DNA, 16 bp, Synthetic construct
  source 1..16: mol_type = other DNA
agctgaaggcaaccag

SEQ ID NO: 94 - DNA, 16 bp, Synthetic construct
  source 1..16: mol_type = other DNA
agagttgacatgggca

SEQ ID NO: 95 - DNA, 16 bp, Synthetic construct
  source 1..16: mol_type = other DNA
ccagtagtcaatatta

SEQ ID NO: 96 - DNA, 16 bp, Synthetic construct
  source 1..16: mol_type = other DNA
ctcaacttgaggacaa

SEQ ID NO: 97 - DNA, 16 bp, Synthetic construct
  source 1..16: mol_type = other DNA
agtatattcagtccaa

SEQ ID NO: 98 - DNA, 16 bp, Synthetic construct
  source 1..16: mol_type = other DNA
tggcaagcatcctgta

SEQ ID NO: 99 - DNA, 16 bp, Synthetic construct
  source 1..16: mol_type = other DNA
ctcaatccatggcagc

SEQ ID NO: 100 - DNA, 16 bp, Synthetic construct
  source 1..16: mol_type = other DNA
accaagtttcttcagc

SEQ ID NO: 101 - DNA, 13 bp, Synthetic construct
  source 1..13: mol_type = other DNA
gcattggtattca

SEQ ID NO: 102 - DNA, 20 bp, Synthetic construct
  source 1..20: mol_type = other DNA
ttcagcattggtattcagtg

SEQ ID NO: 103 - DNA, 16 bp, Synthetic construct
  source 1..16: mol_type = other DNA
cagcattggtattcag

SEQ ID NO: 104 - DNA, 15 bp, Synthetic construct
  source 1..15: mol_type = other DNA
cagcattggtattca

SEQ ID NO: 105 - DNA, 14 bp, Synthetic construct
  source 1..14: mol_type = other DNA
agcattggtattca

SEQ ID NO: 106 - DNA, 12 bp, Synthetic construct
  source 1..12: mol_type = other DNA
gcattggtattc

SEQ ID NO: 107 - DNA, 14 bp, Synthetic construct
  source 1..14: mol_type = other DNA
tgctacaaaaccca

SEQ ID NO: 108 - DNA, 16 bp, Synthetic construct
  source 1..16: mol_type = other DNA
cccattgtcacactcc

SEQ ID NO: 109 - DNA, 18 bp, Synthetic construct
  source 1..18: mol_type = other DNA
cggcatgtctattttgta

SEQ ID NO: 110 - DNA, 20 bp, Synthetic construct
  source 1..20: mol_type = other DNA
ggctaaatcgctccaccaag

SEQ ID NO: 111 - DNA, 20 bp, Synthetic construct
  source 1..20: mol_type = other DNA
ctctagcgtcttaaagccga

SEQ ID NO: 112 - DNA, 20 bp, Synthetic construct
  source 1..20: mol_type = other DNA
gctgcatgatctccttggcg

SEQ ID NO: 113 - DNA, 20 bp, Synthetic construct
  source 1..20: mol_type = other DNA
acgttgaggggcatcgtcgc

SEQ ID NO: 114 - DNA, 20 bp, Synthetic construct
  source 1..20: mol_type = other DNA
cggttagaagactcatcttt

SEQ ID NO: 115 - DNA, 30 bp, Synthetic construct
  source 1..30: mol_type = other DNA
ctccaacatcaaggaagatggcatttctag

SEQ ID NO: 116 - DNA, 23 bp, Synthetic construct
  source 1..23: mol_type = other DNA
gaatattaacanactgacaagtc

SEQ ID NO: 117 - DNA, 21 bp, Synthetic construct
  source 1..21: mol_type = other DNA
cgttgatanttctgccatnct

SEQ ID NO: 118 - DNA, 19 bp, Synthetic construct
  source 1..19: mol_type = other DNA
gccatggttttttctcagg

SEQ ID NO: 119 - DNA, 20 bp, Synthetic construct
  source 1..20: mol_type = other DNA
cctgccctttgttctagttg

SEQ ID NO: 120 - DNA, 19 bp, Synthetic construct
  source 1..19: mol_type = other DNA
gggtctgcagcgggatggt

SEQ ID NO: 121 - DNA, 21 bp, Synthetic construct
  source 1..21: mol_type = other DNA
gttactacttccacctgcctg

SEQ ID NO: 122 - DNA, 25 bp, Synthetic construct
  source 1..25: mol_type = other DNA
tatccggagggctcgccatgctgct

SEQ ID NO: 123 - DNA, 21 bp, Synthetic construct
  source 1..21: mol_type = other DNA
gtcgccccttctccccgcagc

SEQ ID NO: 124 - DNA, 18 bp, Synthetic construct
  source 1..18: mol_type = other DNA
ggaccctcctccggagcc

SEQ ID NO: 125 - DNA, 23 bp, Synthetic construct
  source 1..23: mol_type = other DNA
accaggcgtctcgtggggcacat

SEQ ID NO: 126 - DNA, 18 bp, Synthetic construct
  source 1..18: mol_type = other DNA
tctcccagcgtgcgccat

SEQ ID NO: 127 - DNA, 15 bp, Synthetic construct
  source 1..15: mol_type = other DNA
gtgctccattgatgc

SEQ ID NO: 128 - DNA, 20 bp, Synthetic construct
  source 1..20: mol_type = other DNA
tcccgcctgtgacatgcatt

SEQ ID NO: 129 - DNA, 21 bp, Synthetic construct
  source 1..21: mol_type = other DNA
cagcagcagagtcttcatcat

SEQ ID NO: 130 - DNA, 20 bp, Synthetic construct
  source 1..20: mol_type = other DNA
gggacgcggcgctcggtcat

SEQ ID NO: 131 - DNA, 20 bp, Synthetic construct
  source 1..20: mol_type = other DNA
ccacaagctgtccagtctaa

SEQ ID NO: 132 - DNA, 20 bp, Synthetic construct
  source 1..20: mol_type = other DNA
ccgcagccatgcgctcttgg

SEQ ID NO: 133 - DNA, 20 bp, Synthetic construct
  source 1..20: mol_type = other DNA
tcagggcattctttccattc

SEQ ID NO: 134 - DNA, 20 bp, Synthetic construct
  source 1..20: mol_type = other DNA
cgaaggaaacaatactccga

SEQ ID NO: 135 - DNA, 20 bp, Synthetic construct
  source 1..20: mol_type = other DNA
gacagcagccgcagcagaaa

SEQ ID NO: 136 - DNA, 20 bp, Synthetic construct
  source 1..20: mol_type = other DNA
tggaaaggcttatacccctc

SEQ ID NO: 137 - DNA, 20 bp, Synthetic construct
  source 1..20: mol_type = other DNA
tcaaggaagatggcatttct

SEQ ID NO: 138 - RNA, 16 nt, Synthetic construct
  source 1..16: mol_type = other RNA
gtggctaacagaagct

SEQ ID NO: 139 - RNA, 25 nt, Synthetic construct
  source 1..25: mol_type = other RNA
tttgccgctgcccaatgccatcctg

SEQ ID NO: 140 - DNA, 18 bp, Synthetic construct
  source 1..18: mol_type = other DNA
  modified_base 4: mod_base = OTHER; note = uracil
  modified_base 15: mod_base = OTHER; note = uracil
gcgtgcctcctcactggc

SEQ ID NO: 141 - DNA, 21 bp, Synthetic construct
  source 1..21: mol_type = other DNA
agcttcagtcatgacttcctt

SEQ ID NO: 142 - DNA, 21 bp, Synthetic construct
  source 1..21: mol_type = other DNA
tggtaatccactttcagagga

SEQ ID NO: 143 - DNA, 20 bp, Synthetic construct
  source 1..20: mol_type = other DNA
gttctcgctggtgagtttca

SEQ ID NO: 144 - DNA, 20 bp, Synthetic construct
  source 1..20: mol_type = other DNA
cagctttattagggacagca

SEQ ID NO: 145 - DNA, 21 bp, Synthetic construct
  source 1..21: mol_type = other DNA
cagctttattagggacagcaa

SEQ ID NO: 146 - DNA, 16 bp, Synthetic construct
  source 1..16: mol_type = other DNA
accatgataccacttt

SEQ ID NO: 147 - DNA, 20 bp, Synthetic construct
  source 1..20: mol_type = other DNA
gcttcagtcatgacttcctt

SEQ ID NO: 148 - DNA, 20 bp, Synthetic construct
  source 1..20: mol_type = other DNA
cagctttattagggacagca

SEQ ID NO: 149 - DNA, 14 bp, Synthetic construct
  source 1..14: mol_type = other DNA
tcagtcatgacttc